(19) United States
(12) Patent Application Publication
(10) Pub. No.: US 2024/0425498 A1
(43) Pub. Date: Dec. 26, 2024

(54) PARG INHIBITORY COMPOUNDS

(71) Applicant: FORX THERAPEUTICS AG, Basel (CH)

(72) Inventors: Ulrich Luecking, Berlin (DE); Andreas Goutopoulos, Boston, MA (US); Jin Tian, Hubei (CN); Sotirios Sotiriou, Reinach BL (CH); Luca Iacovino, Rheinfelden (CH); Alena Freudenmann, Riehen (CH); Olivier Querolle, Saint Louis (FR)

(73) Assignee: FORX THERAPEUTICS AG, Basel (CH)

(21) Appl. No.: 18/698,137

(22) PCT Filed: Oct. 3, 2022
(86) PCT No.: PCT/EP2022/077470

§ 371 Date: Apr. 3, 2024

Related U.S. Application Data
(60) Provisional application No. 63/251,916, filed on Oct. 4, 2021.
(60) Provisional application No. 63/321,955, filed on Mar. 21, 2022.
(60) Provisional application No. 63/390,855, filed on Jul. 20, 2022.

(30) Foreign Application Priority Data
Oct. 26, 2021 (EP) 21204879.7
Dec. 22, 2021 (EP) 21217026.0

(51) Int. Cl. C07D 471/04; A61K 31/437; A61K 31/438; A61K 31/439; A61K 31/444; A61K 31/4545; A61K 31/496; A61K 31/4985; A61K 31/499; A61K 31/4995; A61K 31/5377; A61K 31/541; A61K 31/55; A61K 31/551; A61K 31/675; C07D 519/00; C07F 9/6561

(52) U.S. Cl. CPC C07D 471/04; A61K 31/437; A61K 31/438; A61K 31/439; A61K 31/444; A61K 31/4545; A61K 31/496; A61K 31/4985; A61K 31/499; A61K 31/4995; A61K 31/5377; A61K 31/541; A61K 31/55; A61K 31/551; A61K 31/675; C07D 519/00; C07F 9/6561

(57) ABSTRACT

The present invention relates to a compound of formula (I) or an enantiomer, diastereoisomer, tautomer, pharmaceutically acceptable solvate, pharmaceutically acceptable crystal form, pharmaceutically acceptable salt or a prodrug thereof. The present invention further relates to the compound of formula (I) of the present invention for use in therapy. Instant compounds are particularly useful as PARC inhibitors, and can be used in a method of treatment of a proliferative disorder, preferably of cancer.

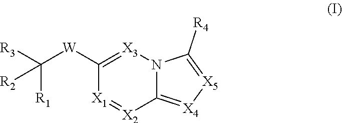

### FIELD OF THE INVENTION

The present invention relates to a compound of formula (I):

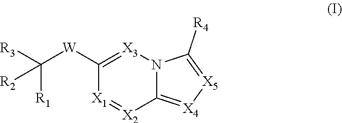

or an enantiomer, diastereoisomer, tautomer, pharmaceutically acceptable solvate, pharmaceutically acceptable crystal form, pharmaceutically acceptable salt or a prodrug thereof. The present invention further relates to the compound of formula (I) of the present invention for use in therapy. Instant compounds are particularly useful as PARG inhibitors, and can be used in a method of treatment of a proliferative disorder, preferably of cancer.

### BACKGROUND OF THE INVENTION

Cancer is a leading cause of death worldwide. Although progression-free survival and overall survival of cancer patients has improved over the past two decades, millions of cancer patients still have few therapeutic options and poor survival outcomes (Jemal et al., J. Natl. Cancer Inst. 2017, 109, 1975).

DNA replication stress (DRS) is a hallmark of cancer cells and a major source of genomic instability (a) Halazonetis et al., Science 2008, 319, 1352; b) Negrini et al., Nat. Rev. Mol. Cell Biol. 2010, 11, 220). In broad terms, DRS refers to the deregulation of DNA replication and cell cycle progression. DRS can be induced from endogenous or exogenous causes such as oncogene activation and chemotherapeutics, respectively (Zeman and Cimprich, Nat. Cell Biol. 2013, 16, 2). At the level of the replication fork, DRS leads to replication fork stalling, disengagement of the replisome and eventually collapse. Several DNA repair proteins are involved in replication fork stability, protection, and restart under DRS conditions (a) Costantino et al., Science 2014, 343, 88; b) Scully et al., Curr. Opin. Genet. Dev. 2021 71, 154).

Poly(ADP)ribosylation (PARylation) is a transient and reversible post-translational modification that occurs at DNA damaged sites and is catalyzed by the poly (ADP-ribose) polymerase (PARP) family of proteins (Cohen and Chang, Nat. Chem. Biol. 2018, 14, 236). PARylation of various DNA repair proteins leads to their activation. Degradation of the poly(ADP) ribose chains is mediated primarily by the poly(ADP-ribose) glycohydrolase (PARG) protein. DNA damage dependent PARylation/dePARylation is a rapid and dynamic process which needs to be well regulated since imbalances between the two processes can lead to DNA damage.

Human PARG encodes a 111 kDa protein of 976 amino acids. It contains a N-terminal regulatory domain, a catalytic domain and an ADP-ribose binding macrodomain. Five human PARG transcripts have been identified. Full length PARG is mostly nuclear; the smaller isoforms localize primarily to the cytoplasm. PARG functions primarily as an exo-hydrolase and it releases mainly mono(ADP-ribose) by hydrolyzing the α-O-glycosidic ribose-ribose bond in PAR. PARG can also act as an endo-hydrolase. PARG preferentially degrades long and linear PAR chains whereas its activity with small and branched PAR chains is significantly reduced (O'Sullivan et al., Nat. Commun. 2019, 10, 1182).

Although PARG is the dominant cellular PAR degrading enzyme, it cannot act on the terminal protein-ribose bond. Additional hydrolases such as terminal ADP-ribose protein glycohydrolase (TARG1) and ADP-ribosylhydrolase 3 (ARH3) are also known to catalyze PAR-degradation. TARG1 and ARH3 complete the reversal of PARylation by removing protein-bound mono(ADP-ribose) moieties (a) Fontana et al., Elife 2017, doi: 10.7554/eLife.28533; b) Rack et al., Genes Dev. 2020, 34, 263). TARG1 is located in the nucleus and cytoplasm. ARH3 is found primarily in the cytoplasm but it can also be found in the mitochondria and in the nucleus (Rack et al., Genes Dev. 2020, 34, 263).

Genomic aberrations targeting tumor suppressor genes or oncogenes, often make cancer cells dependent on specific DNA repair pathways. For instance, it is well known that PARP inhibitors are particularly effective against tumors carrying mutations in the BRCA1 and BRCA2 genes (a) Bryant et al., Nature 2005, 434, 913; b) Farmer et al., Nature 2005, 434, 917). Targeting synthetic lethal interactions like the one between PARP and BRCA is an attractive novel therapeutic approach for cancer treatment.

PARG participates in DNA replication and in various DNA repair mechanisms including single-strand break (SSB) repair and replication fork restart. PARG inhibitors have shown synthetic lethal phenotype in cells with high levels of DRS caused by low expression of genes involved in DNA replication and/or replication fork stability (Pillay et al., Cancer Cell. 2019, 35, 519). Moreover, PARG inactivation, depletion or inhibition sensitizes cells to irradiation and to DNA damaging agents such as alkylating agents (e.g. temozolomide and methyl methanesulfonate) (a) Fujihara et al., Curr. Cancer Drug Targets 2009, 9, 953; b) Gogola et al., Cancer Cell 2018, 33, 1078; c) Houl et al., Nat Commun. 2019, 10, 5654).

Given the therapeutic potential of PARG inhibitors in cancer treatment, there is an increased need for the development of highly potent and selective PARG inhibitors beyond the ones that have already been described (a) James et al., ACS Chem. Biol. 2016, 11, 3179; b) Waszkowycz et al., J. Med. Chem. 2018, 61, 10767).

Certain compounds that are useful as PARG inhibitors are further disclosed in documents WO 2016/092326, WO 2016/097749 and WO 2021/055744.

Document US 2019/233411 discloses certain Gcn2 inhibitors and uses thereof.

Document WO 2009/050183 discloses certain imidazo[1,2-a]pyridine derivatives which are useful for treating diseases mediated by the ALK-5 and/or ALK-4 receptor.

### SUMMARY OF THE INVENTION

It was an objective technical problem of the present invention to provide compounds that are cell-permeable inhibitors of PARG. The technical problem of the present invention is solved by the embodiments described herein and as characterized by the claims.

Accordingly, in a first embodiment, the present invention provides a compound of formula (I):

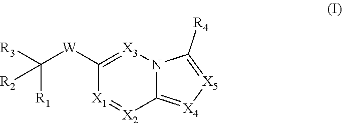

or an enantiomer, diastereoisomer, tautomer, pharmaceutically acceptable solvate, pharmaceutically acceptable crystal form, pharmaceutically acceptable salt or a prodrug thereof. It is understood that thought the present description the term “a compound of formula (I)” preferably encompasses also a compound of formula (Ia) to (Ibo), unless indicated to the contrary.

A further embodiment of the present invention relates to a pharmaceutical composition comprising the compound of formula (I) or a pharmaceutically acceptable salt, hydrate or solvate thereof, and a pharmaceutically acceptable carrier.

In a further embodiment, the present invention relates to the compound of formula (I) of the present invention or a pharmaceutically acceptable salt, hydrate or solvate thereof, or a pharmaceutical composition of the present invention, for use in therapy.

The compounds of formula (I) are useful for treating a disease or disorder in which PARG activity is implicated.

The compounds of formula (I) are useful for a method of treating a proliferative disorder. In a preferred embodiment of the present invention, the proliferative disorder is cancer, preferably a human cancer.

### Definitions

The following definitions apply throughout the present specification and the claims, unless specifically indicated otherwise.

The term “hydrogen” is herein used to refer to protium, deuterium and/or tritium, preferably to protium. Accordingly, the term “non-hydrogen atom” refers to any atoms that is not hydrogen, i.e. that is not protium, deuterium or tritium.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

Preferably, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “arylene” refers to an aryl group, as defined herein above, but having two points of attachment, i.e. a divalent aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Arylene” may, e.g., refer to phenylene (e.g., phen-1,2-diyl, phen-1,3-diyl, or phen-1,4-diyl), naphthylene (e.g., naphthalen-1,2-diyl, naphthalen-1,3-diyl, naphthalen-1,4-diyl, naphthalen-1,5-diyl, naphthalen-1,6-diyl, naphthalen-1,7-diyl, naphthalen-2,3-diyl, naphthalen-2,5-diyl, naphthalen-2,6-diyl, naphthalen-2,7-diyl, or naphthalen-2,8-diyl), 1,2-dihydronaphthylene, 1,2,3,4-tetrahydronaphthylene, indanylene, indenylene, anthracenylene, phenanthrenylene, 9H-fluorenylene, or azulenylene. Unless defined otherwise, an “arylene” preferably has 6 to 14 ring atoms, more preferably 6 to 10 ring atoms, even more preferably refers to phenylene or naphthylene, and most preferably refers to phenylene (particularly phen-1,4-diyl).

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “heteroarylene” refers to a heteroaryl group, as defined herein above, but having two points of attachment, i.e. a divalent aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three, or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroarylene” may, e.g., refer to thienylene (i.e., thiophenylene; e.g., thien-2,3-diyl, thien-2,4-diyl, or thien-2,5-diyl), benzo[b]thienylene, naphtho[2,3-b]thienylene, thianthrenylene, furylene (i.e., furanylene; e.g., furan-2,3-diyl, furan-2,4-diyl, or furan-2,5-diyl), benzofuranylene, isobenzofuranylene, chromanylene, chromenylene, isochromenylene, chromonylene, xanthenylene, phenoxathiinylene, pyrrolylene, imidazolylene, pyrazolylene, pyridylene (i.e., pyridinylene), pyrazinylene, pyrimidinylene, pyridazinylene, indolylene, isoindolylene, indazolylene, indolizinylene, purinylene, quinolylene, isoquinolylene, phthalazinylene, naphthyridinylene, quinoxalinylene, cinnolinylene, pteridinylene, carbazolylene, R-carbolinylene, phenanthridinylene, acridinylene, perimidinylene, phenanthrolinylene, phenazinylene, thiazolylene (e.g., thiazol-2,4-diyl, thiazol-2,5-diyl, or thiazol-4,5-diyl), isothiazolylene (e.g., isothiazol-3,4-diyl, isothiazol-3,5-diyl, or isothiazol-4,5-diyl), phenothiazinylene, oxazolylene (e.g., oxazol-2,4-diyl, oxazol-2,5-diyl, or oxazol-4,5-diyl), isoxazolylene (e.g., isoxazol-3,4-diyl, isoxazol-3,5-diyl, or isoxazol-4,5-diyl), oxadiazolylene (e.g., 1,2,4-oxadiazol-3,5-diyl, 1,2,5-oxadiazol-3,4-diyl, or 1,3,4-oxadiazol-2,5-diyl), thiadiazolylene (e.g., 1,2,4-thiadiazol-3,5-diyl, 1,2,5-thiadiazol-3,4-diyl, or 1,3,4-thiadiazol-2,5-diyl), phenoxazinylene, pyrazolo[1,5-a]pyrimidinylene, 1,2-benzoisoxazolylene, benzothiazolylene, benzothiadiazolylene, benzoxazolylene, benzisoxazolylene, benzimidazolylene, benzo[b]thiophenylene (i.e., benzothienylene), triazolylene (e.g., 1H-1,2,3-triazolylene, 2H-1,2,3-triazolylene, 1H-1,2,4-triazolylene, or 4H-1,2,4-triazolylene), benzotriazolylene, 1H-tetrazolylene, 2H-tetrazolylene, triazinylene (e.g., 1,2,3-triazinylene, 1,2,4-triazinylene, or 1,3,5-triazinylene), furo[2,3-c]pyridinylene, dihydrofuropyridinylene (e.g., 2,3-dihydrofuro[2,3-c]pyridinylene or 1,3-dihydrofuro[3,4-c]pyridinylene), imidazopyridinylene (e.g., imidazo[1,2-a]pyridinylene or imidazo[3,2-a]pyridinylene), quinazolinylene, thienopyridinylene, tetrahydrothienopyridinylene (e.g., 4,5,6,7-tetrahydrothieno[3,2-c]pyridinylene), dibenzofuranylene, 1,3-benzodioxolylene, benzodioxanylene (e.g., 1,3-benzodioxanylene or 1,4-benzodioxanylene), or coumarinylene. Unless defined otherwise, the term “heteroarylene” preferably refers to a divalent 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroarylene” refers to a divalent 5 or 6 membered monocyclic ring comprising one or more (e.g., one, two or three) ring heteroatoms independently selected from O, S, and N, wherein one or more S ring atoms (if present) and/or one or more N ring atoms (if present) are optionally oxidized, and wherein one or more carbon ring atoms are optionally oxidized. A “heteroarylene”, including any of the specific heteroarylene groups described herein, may be attached through two carbon ring atoms, particularly through those two carbon ring atoms that have the greatest distance from one another (in terms of the number of ring atoms separating them by the shortest possible connection) within one single ring or within the entire ring system of the corresponding heteroarylene.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members (e.g., cyclopropyl or cyclohexyl). As used herein, the term “cycloalkylene” refers to a cycloalkyl group, as defined herein above, but having two points of attachment, i.e. a divalent saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkylene” may, e.g., refer to cyclopropylene (e.g., cyclopropan-1,1-diyl or cyclopropan-1,2-diyl), cyclobutylene (e.g., cyclobutan-1,1-diyl, cyclobutan-1,2-diyl, or cyclobutan-1,3-diyl), cyclopentylene (e.g., cyclopentan-1,1-diyl, cyclopentan-1,2-diyl, or cyclopentan-1,3-diyl), cyclohexylene (e.g., cyclohexan-1,1-diyl, cyclohexan-1,2-diyl, cyclohexan-1,3-diyl, or cyclohexan-1,4-diyl), cycloheptylene, decalinylene (i.e., decahydronaphthylene), or adamantylene. Unless defined otherwise, “cycloalkylene” preferably refers to a C₃₋₁₁ cycloalkylene, and more preferably refers to a C₃₋₇ cycloalkylene. A particularly preferred “cycloalkylene” is a divalent monocyclic saturated hydrocarbon ring having 3 to 7 ring members (e.g., cyclopropylene or cyclohexylene).

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, 1,1-dioxothianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

Preferably, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, 1,1-dioxothianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from 0, S and N, wherein one or more S ring atoms (if present) and/or one or more N ring atoms (if present) are optionally oxidized, and wherein one or more carbon ring atoms are optionally oxidized. As used herein, the term “heterocycloalkylene” refers to a heterocycloalkyl group, as defined herein above, but having two points of attachment, i.e. a divalent saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, 1,1-dioxothianylene, thiepanylene, decahydroquinolinylene, decahydroisoquinolinylene, or 2-oxa-5-aza-bicyclo[2.2.1]hept-5-ylene. Unless defined otherwise, “heterocycloalkylene” preferably refers to a divalent 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

Preferably, the term “heterocycloalkylene” refers to a heterocycloalkyl group, as defined herein above, but having two points of attachment, i.e. a divalent saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, 1,1-dioxothianylene, thiepanylene, decahydroquinolinylene, decahydroisoquinolinylene, or 2-oxa-5-aza-bicyclo[2.2.1]hept-5-ylene. Unless defined otherwise, “heterocycloalkylene” preferably refers to a divalent 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “N-heterocycloalkyl” refers to the heterocycloalkyl groups as defined hereinabove wherein said heterocycloalkyl includes at least one nitrogen atom which serves as an attachment point of said heterocycloalkyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “cycloalkenylene” refers to a cycloalkenyl group, as defined hereinabove, but having two points of attachment, i.e. a divalent unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from 0, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

Preferably, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two 0 atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “heterocycloalkenylene” refers to a heterocycloalkenyl group, as defined hereinabove, as defined hereinabove, but having two points of attachment, i.e. a divalent unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si and the remaining ring atoms are carbon atoms, wherein one or more S ring atoms (if present) and/or one or more N ring atoms (if present) and/or one or more P ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring.

Preferably, the term “heterocycloalkenylene” refers to a heterocycloalkenyl group, as defined hereinabove, as defined hereinabove, but having two points of attachment, i.e. a divalent unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring.

As used herein, the term “halogen” refers to fluoro (—F), chloro (—Cl), bromo (—Br), or iodo (—I). As it is to be understood for the skilled person, the terms “halogen” and “halo” may be used interchangeably.

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

### DETAILED DESCRIPTION OF THE INVENTION

The invention is described in detail in the following. It is to be understood that the present invention specifically relates to each and every combination of features and embodiments described herein, including any combination of general and/or preferred features/embodiments.

In a first embodiment, the present invention relates to a compound of formula (I):

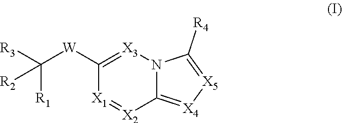

or an enantiomer, diastereoisomer, tautomer, pharmaceutically acceptable solvate, pharmaceutically acceptable crystal form, pharmaceutically acceptable salt or a prodrug thereof.
R₁ is selected from the group consisting of hydrogen, chloro, fluoro, cyano, formyl, (C₁₋₂)alkyl, (C₂)alkenyl, (C₂)alkynyl, (C₁₋₂)haloalkyl, —(C₁₋₂ alkylene)-OH and —(C₁₋₂ alkylene)-O—(C₁₋₂ alkyl), preferably from the group consisting of chloro, fluoro, cyano, formyl, (C₁₋₂)alkyl, (C₂)alkenyl, (C₂)alkynyl, (C₁₋₂)haloalkyl, —(C₁₋₂ alkylene)-OH and —(C₁₋₂ alkylene)-O—(C₁₋₂ alkyl). Preferably, R₁ is selected from the group consisting of hydrogen, chloro, fluoro, cyano, formyl, (C₁₋₂)alkyl, (C₂)alkenyl, (C₂)alkynyl and (C₁₋₂)haloalkyl, preferably from the group consisting of chloro, fluoro, cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)alkyl, and (C₁₋₂)haloalkyl. Preferably, (C₁₋₂)alkyl as discussed herein is methyl. Preferably, (C₁₋₂)haloalkyl as discussed herein is fluoromethyl. Thus, preferably R₁ is selected from the group consisting of cyano, methyl and fluoromethyl. More preferably, R₁ is cyano. However, in an alternative preferred embodiment, R₁ is methyl, in a particularly preferred alternative embodiment wherein R₁ is methyl, R₁ is CD₃. In again an alternative preferred embodiment, R₁ is fluoromethyl.
R₂ and R₃ are independently each (C₁₋₂)alkyl or (C₁₋₂)haloalkyl, preferably methyl, or R₂ and R₃ together with the carbon atom to which they are attached form cyclopropyl. Preferably, R₂ and R₃ together with the carbon atom to which they are attached form cyclopropyl.
W is selected from —NHS(O)_{y}—, —S(O)_{y}NH—, —NHS(O)(NH)—, —NHS(O)(NCH₃)—, —S(O)(NH)—NH—, —S(O)(NCH₃)—NH—, wherein y is 1 or 2. Preferably, y is 2. Thus, in a preferred embodiment, W is selected from —NHS(O)₂—, —S(O)₂NH—, —NHS(O)(NH)—, and —S(O)(NH)—NH—. More preferably, W is selected from —NHS(O)₂—, and —S(O)₂NH—, even more preferably W is —NHS(O)₂—. Preferably as understood herein, the left side of W as defined herein is attached to the carbon atom that carries R₁, R₂ and R₃, and the right side of W as defined herein is attached to the ring system shown in formula (I). In one preferred embodiment, W is —NHS(O)₂— or —NHS(O)(NCH₃)—. In one preferred embodiment, W is —NHS(O)(NCH₃)—.
X₁ and X₃ are independently selected from the group consisting of N, CH, C(C₁₋₂ alkyl), CCI and CF, preferably independently selected from the group consisting of N, CH and CF. Preferably, X₁ is CF or CH and X₃ is CH, more preferably X₁ and X₃ are each CH. However, in an alternative preferred embodiment, X₁ is CF and X₃ is CH.
X₂ is N or C—Y_{C2}—R_{C2}, preferably X₂ is C—Y_{C2}—R_{C2}.
Y_{C2} is selected from a covalent bond, C₁₋₅ alkylene, C₂₋₅ alkenylene, C₂₋₅ alkynylene, cycloalkylene, cycloalkenylene, heterocycloalkylene and heterocycloalkenylene, wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and further wherein said cycloalkylene, said cycloalkenylene, said heterocycloalkylene and said heterocycloalkenylene are each optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), and —(C₁₋₅ alkylene)N(C₁₋₅ alkyl)CON(C₁₋₅ alkyl)(C₁₋₅ alkyl), preferably selected from halogen, CN, OH, C₁₋₅ alkyl, O(C₁₋₅ alkyl), SH, S(C₁₋₅ alkyl), NH₂, NH(C₁₋₅ alkyl), and N(C₁₋₅ alkyl)(C₁₋₅ alkyl). Preferably, Y_{C2} is selected from a covalent bond, C₁₋₅ alkylene, C₂₋₅ alkenylene, C₂₋₅ alkynylene, cycloalkylene and heterocycloalkylene wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and further wherein said cycloalkylene and said heterocycloalkylene are each optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), and —(C₁₋₅ alkylene)N(C₁₋₅ alkyl)CON(C₁₋₅ alkyl)(C₁₋₅ alkyl), preferably selected from halogen, CN, OH, C₁₋₅ alkyl, O(C₁₋₅ alkyl), SH, S(C₁₋₅ alkyl), NH₂, NH(C₁₋₅ alkyl), and N(C₁₋₅ alkyl)(C₁₋₅ alkyl). More preferably, Y_{C2} is selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—. More preferably, Y_{C2} is selected from a covalent bond, —(C₁₋₃ alkylene)-, —CO—(C₁₋₃ alkylene)-, (C₁₋₃ alkylene)-CO—, —CONH—(C₁₋₃ alkylene)-, —(C₁₋₃ alkylene)-CONH—, —NHCO—(C₁₋₃ alkylene)-, —(C₁₋₃ alkylene)-NHCO—, —NH—(C₁₋₃ alkylene)-, —(C₁₋₃ alkylene)-NH—, —N(C₁₋₅ alkyl)-, —O—(C₁₋₃ alkylene)-, —(C₁₋₃ alkylene)-O—, SO₂—(C₁₋₃ alkylene)-, —(C₁₋₃ alkylene)SO₂—, —CONH—, —NHCO—, —NH—, —O—, —CO— and SO₂—. C₁₋₃ alkylene is herein preferably a —CH₂— group.
R_{C2} is selected from hydrogen, halo, —OH, —NH₂, —SH, —CN, C₁₋₁₂ alkyl, C₂₋₁₂ alkenyl, C₂₋₁₂ alkynyl, cycloalkyl, cycloalkenyl, heterocycloalkyl, heterocycloalkenyl, aryl, and heteroaryl. Preferably, R_{C2} is selected from hydrogen, halo, —OH, —NH₂, —SH, —CN, C₁₋₁₂ alkyl, C₂₋₁₂ alkenyl, C₂₋₁₂ alkynyl, cycloalkyl, heterocycloalkyl, aryl, and heteroaryl. More preferably, R_{C2} is selected from hydrogen, cycloalkyl, heterocycloalkyl, aryl, and heteroaryl. Even more preferably, R_{C2} is selected from cycloalkyl, heterocycloalkyl, aryl, and heteroaryl. Even more preferably, R_{C2} is selected from heterocycloalkyl, aryl, and heteroaryl. Said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —O(C₁₋₅ alkyl)-, —O(C₁₋₅ haloalkyl)-, C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl). Said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl), —(C₁₋₅ alkyl), —C(₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-C(C₁₋₅ alkyl), —(C₁₋₅ alkylene)-C(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(O)(C₁₋₅ alkyl), S(O)₂(C₁₋₅ alkyl), S(O)(NH)(C₁₋₅ alkyl), S(O)(N(C₁₋₅ alkyl))(C₁₋₅ alkyl), —N═S(O)(C₁₋₅ alkyl)(C₁₋₅ alkyl), —S(C₁₋₅ haloalkyl), NH-2, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), COO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), NHCOO(C₁₋₅ alkyl), N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)S(O)(C₁₋₅ alkyl), —(C₁₋₅ alkylene)S(O)₂(C₁₋₅ alkyl), —(C₁₋₅ alkylene)S(O)(NH)(C₁₋₅ alkyl), —(C₁₋₅ alkylene)S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), —(C₁₋₅ alkylene)N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)NHCOO(C₁₋₅ alkyl), and —(C₁₋₅ alkylene)N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

Thus, preferably, —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂₋₁₂ alkenyl, —O—C₂₋₁₂ alkynyl, —NH—C₂₋₁₂ alkynyl, —N(C₁₋₅ alkyl)-C₂₋₁₂ alkynyl, (C₀₋₃ alkylene)-cycloalkyl, —CO—(C₀₋₃ alkylene)cycloalkyl, (C₀₋₃ alkylene)-CO-cycloalkyl, —CONH—(C₀₋₃ alkylene)cycloalkyl, —(C₀₋₃ alkylene)-CONH-cycloalkyl, —NHCO—(C₀₋₃ alkylene)cycloalkyl, (C₀₋₃ alkylene)-NHCO-cycloalkyl, —NH—(C₀₋₃ alkylene)cycloalkyl, —(C₀₋₃ alkylene)-NH-cycloalkyl, —O—(C₀₋₃ alkylene)cycloalkyl, —(C₀₋₃ alkylene)-O-cycloalkyl, —SO₂—(C₀₋₃ alkylene)cycloalkyl, —(C₀₋₃ alkylene)-SO₂-cycloalkyl, —CONH-cycloalkyl, —NHCO-cycloalkyl, —NH-cycloalkyl, —O-cycloalkyl, —CO-cycloalkyl, —SO₂-cycloalkyl, (C₀₋₃ alkylene)-cycloalkenyl, —CO—(C₀₋₃ alkylene)cycloalkenyl, (C₀₋₃ alkylene)-CO-cycloalkenyl, —CONH—(C₀₋₃ alkylene)cycloalkenyl, —(C₀₋₃ alkylene)-CONH-cycloalkenyl, —NHCO—(C₀₋₃ alkylene)cycloalkenyl, (C₀₋₃ alkylene)-NHCO-cycloalkenyl, —NH—(C₀₋₃ alkylene)cycloalkenyl, —(C₀₋₃ alkylene)-NH-cycloalkenyl, —O—(C₀₋₃ alkylene)cycloalkenyl, —(C₀₋₃ alkylene)-O-cycloalkenyl, —SO₂—(C₀₋₃ alkylene)cycloalkenyl, —(C₀₋₃ alkylene)-SO₂-cycloalkenyl, —CONH-cycloalkenyl, —NHCO-cycloalkenyl, —NH— cycloalkenyl, —O-cycloalkenyl, —CO-cycloalkenyl, —SO₂-cycloalkenyl, —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, —(C₀₋₃ alkylene)-O-heterocycloalkyl, —SO₂—(C₀₋₃ alkylene)heterocycloalkyl, —(C₀₋₃ alkylene)-SO₂-heterocycloalkyl, —CONH-heterocycloalkyl, —NHCO-heterocycloalkyl, —NH-heterocycloalkyl, —O-heterocycloalkyl, —CO— heterocycloalkyl, —SO₂-heterocycloalkyl, —(C₀₋₃ alkylene)-heterocycloalkenyl, —CO—(C₀₋₃ alkylene)heterocycloalkenyl, —(C₀₋₃ alkylene)-CO-heterocycloalkenyl, —CONH—(C₀₋₃ alkylene)heterocycloalkenyl, —(C₀₋₃ alkylene)-CONH-heterocycloalkenyl, —NHCO—(C₀₋₃ alkylene)heterocycloalkenyl, —(C₀₋₃ alkylene)-NHCO-heterocycloalkenyl, —NH—(C₀₋₃ alkylene)heterocycloalkenyl, —(C₀₋₃ alkylene)-NH-heterocycloalkenyl, —O—(C₀₋₃ alkylene) heterocycloalkenyl, —(C₀₋₃ alkylene)-O-heterocycloalkenyl, —SO₂—(C₀₋₃ alkylene)heterocycloalkenyl, —(C₀₋₃ alkylene)-SO₂-heterocycloalkenyl, —CONH-heterocycloalkenyl, —NHCO-heterocycloalkenyl, —NH— heterocycloalkenyl, —O-heterocycloalkenyl, —CO-heterocycloalkenyl, —SO₂-heterocycloalkenyl, (C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, (C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, preferably —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂₋₁₂ alkenyl, —O—C₂₋₁₂ alkynyl, —NH—C₂₋₁₂ alkynyl, —N(C₁₋₅ alkyl)-C₂₋₁₂ alkynyl, (C₀₋₃ alkylene)-cycloalkyl, —CO—(C₀₋₃ alkylene)cycloalkyl, (C₀₋₃ alkylene)-CO-cycloalkyl, —CONH—(C₀₋₃ alkylene)cycloalkyl, —(C₀₋₃ alkylene)-CONH-cycloalkyl, —NHCO—(C₀₋₃ alkylene)cycloalkyl, (C₀₋₃ alkylene)-NHCO-cycloalkyl, —NH—(C₀₋₃ alkylene)cycloalkyl, —(C₀₋₃ alkylene)-NH-cycloalkyl, —O—(C₀₋₃ alkylene)cycloalkyl, —(C₀₋₃ alkylene)-O-cycloalkyl, —SO₂—(C₀₋₃ alkylene)cycloalkyl, —(C₀₋₃ alkylene)-SO₂-cycloalkyl, —CONH-cycloalkyl, —NHCO-cycloalkyl, —NH-cycloalkyl, —O-cycloalkyl, —CO— cycloalkyl, —SO₂-cycloalkyl, —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, —(C₀₋₃ alkylene)-O-cycloalkyl, —SO₂—(C₀₋₃ alkylene)heterocycloalkyl, —(C₀₋₃ alkylene)-SO₂-heterocycloalkyl, —CONH-heterocycloalkyl, —NHCO-heterocycloalkyl, —NH-heterocycloalkyl, —O-heterocycloalkyl, —CO— heterocycloalkyl, —SO₂-heterocycloalkyl, (C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, (C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —O(C₁₋₅ alkyl), —O(C₁₋₅ haloalkyl), —C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl) and wherein said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₇ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

More preferably, —Y_{C2}—R_{C2} is selected from —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, (C₀₋₃ alkylene)-O-cycloalkyl, (C₀₋₃ alkylene)-O-heterocycloalkyl, —SO₂—(C₀₋₃ alkylene)heterocycloalkyl, —(C₀₋₃ alkylene)-SO₂-heterocycloalkyl, —CONH-heterocycloalkyl, —NHCO— heterocycloalkyl, —NH-heterocycloalkyl, —O-heterocycloalkyl, —CO-heterocycloalkyl, —SO₂-heterocycloalkyl, —(C₀₋₃ alkylene)-heterocycloalkenyl, —CO—(C₀₋₃ alkylene)heterocycloalkenyl, —(C₀₋₃ alkylene)-CO-heterocycloalkenyl, —CONH—(C₀₋₃ alkylene)heterocycloalkenyl, —(C₀₋₃ alkylene)-CONH-heterocycloalkenyl, —NHCO—(C₀₋₃ alkylene)heterocycloalkenyl, —(C₀₋₃ alkylene)-NHCO-heterocycloalkenyl, —NH—(C₀₋₃ alkylene)heterocycloalkenyl, —(C₀₋₃ alkylene)-NH-heterocycloalkenyl, —O—(C₀₋₃ alkylene) heterocycloalkenyl, —(C₀₋₃ alkylene)-O-heterocycloalkenyl, —SO₂—(C₀₋₃ alkylene)heterocycloalkenyl, —(C₀₋₃ alkylene)-SO₂-heterocycloalkenyl, —CONH-heterocycloalkenyl, —NHCO-heterocycloalkenyl, —NH— heterocycloalkenyl, —O-heterocycloalkenyl, —CO-heterocycloalkenyl, —SO₂-heterocycloalkenyl, —(C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, —(C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, preferably —Y_{C2}—R_{C2} is selected from —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, (C₀₋₃ alkylene)-O-cycloalkyl, —SO₂—(C₀₋₃ alkylene)heterocycloalkyl, —(C₀₋₃ alkylene)-SO₂-heterocycloalkyl, —CONH-heterocycloalkyl, —NHCO-heterocycloalkyl, —NH-heterocycloalkyl, —O-heterocycloalkyl, —CO-heterocycloalkyl, —SO₂-heterocycloalkyl, —(C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, —(C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO— heteroaryl, —NH-heteroaryl, —O-heteroaryl, —CO-heteroaryl and —SO₂-heteroaryl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl), —P(O)(O(C₁₋₅ alkyl))(O(C₁₋₅ alkyl)), —P(O)((C₁₋₅ alkyl))(C₁₋₅ alkyl), —(C₁₋₅ alkylene)-CN, —(C₁₋₅ alkylene)-OH, —(C₁₋₅ alkylene)-(C₁₋₅ alkyl), —(C₁₋₅ alkylene)-O(C₁₋₅ haloalkyl), —(C₁₋₅ alkylene)-SH, —(C₁₋₅ alkyl)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

More preferably, —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O-heterocycloalkyl, —CO— heterocycloalkyl, —SO₂-heterocycloalkyl, —(C₀₋₃ alkylene)-heterocycloalkenyl, —CONH-heterocycloalkenyl, —NHCO-heterocycloalkenyl, —NH-heterocycloalkenyl, —O-heterocycloalkenyl, —CO— heterocycloalkenyl, —SO₂-heterocycloalkenyl, —(C₀₋₃ alkylene)aryl, —CONH-aryl, —NHCO-aryl, —NH-aryl, —O-aryl, —CO-aryl, —SO₂-aryl, —(C₀₋₃ alkylene)heteroaryl, —CONH-heteroaryl, —NHCO-heteroaryl, —NH— heteroaryl, —O-heteroaryl, —CO-heteroaryl and —SO₂-heteroaryl, preferably —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)aryl, —CONH-aryl, —NHCO-aryl, —NH-aryl, —O-aryl, —CO-aryl, —SO₂-aryl, —(C₀₋₃ alkylene)heteroaryl, —CONH-heteroaryl, —NHCO— heteroaryl, —NH-heteroaryl, —O-heteroaryl, —CO-heteroaryl and —SO₂-heteroaryl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl), —(C₁₋₅ alkylene)-P(O)((C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haoalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-N(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkyl)CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₄ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

Even more preferably, —Y_{C2}—R_{C2} is selected from —(C₀₋₃ alkylene)-heterocycloalkyl, —(C₀₋₃ alkylene)-heterocycloalkenyl, —(C₀₋₃ alkylene)aryl, and —(C₀₋₃ alkylene)heteroaryl, preferably —Y_{C2}—R_{C2} is selected from —(C₀₋₃ alkylene)-heterocycloalkyl, —(C₀₋₃ alkylene)aryl, and —(C₀₋₃ alkylene)heteroaryl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ ay)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)((C₁₋₅ alkyl))(O(C₁₋₅ alkyl)), —P(O)(O(C₁₋₅ (C₁₋₅))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkyl)-CO—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)(C₁₋₅ alkylene)-P(O)(O(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

Even more preferably, —Y_{C2}—R_{C2} is selected from heterocycloalkyl, heterocycloalkenyl, aryl, and heteroaryl, more preferably heterocycloalkyl, aryl, and heteroaryl, more preferably heterocycloalkyl and heteroaryl, even more preferably, heterocycloalkyl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —C(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ haloalkyl), —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

In one embodiment, —Y_{C2}—R_{C2} is heterocycloalkenyl, wherein said heterocycloalkenyl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

Preferably, if —Y_{C2}—R_{C2} is aryl, —Y_{C2}—R_{C2} is phenyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

Preferably, if —Y_{C2}—R_{C2} is heteroaryl, —Y_{C2}—R_{C2} is imidazolyl, pyridazinyl, thiazolyl, pyridinyl, pyrimidinyl, pyrazinyl, or indazolyl, wherein heteroaryl may be optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

Preferably, if —Y_{C2}—R_{C2} is heterocycloalkyl, —Y_{C2}—R_{C2} is morpholinyl, 1,1-dioxothiomorpholinyl, azetinyl, pyrrolidinyl, piperidinyl, 6-oxo-1,6-dihydropyridinyl, or piperazinyl, wherein heterocycloalkyl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)((C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from, halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl). More preferably, —Y_{C2}—R_{C2} is piperazinyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl)), —(C₁₋₅ alkylene)-P(O)((C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl). Even more preferably, —Y_{C2}—R_{C2} is piperazinyl (preferably N-piperazinyl) optionally substituted (preferably N-substituted) with —CO(C₁₋₅ alkyl), —CONH₂, —CONH(C₁₋₅ alkyl), and —CON(C₁₋₅ alkyl)(C₁₋₅ alkyl). Most preferably, —Y_{C2}—R_{C2} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.

Preferably, if —Y_{C2}—R_{C2} is heterocycloalkenyl, —Y_{C2}—R_{C2} is oxacyclohexenyl or azacyclohexenyl, wherein heterocycloalkenyl is optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(O)(C₁₋₅ alkyl), S(O)₂(C₁₋₅ alkyl), S(O)(NH)(C₁₋₅ alkyl), S(O)(N(C₁₋₅ alkyl))(C₁₋₅ alkyl), —N═S(O)(C₁₋₅ alkyl)(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, COO(C₁₋₅ alkyl), —COO(C₁₋₅ haloalkyl), —COO-cycloalkyl, CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), NHCOO(C₁₋₅ alkyl), N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)S(O)(C₁₋₅ alkyl), —(C₁₋₅ alkylene)S(O)₂(C₁₋₅ alkyl), —(C₁₋₅ alkylene)S(O)(NH)(C₁₋₅ alkyl), —(C₁₋₅ alkylene)S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), —(C₁₋₅ alkylene)N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)NHCOO(C₁₋₅ alkyl), and —(C₁₋₅ alkylene)N(C₁₋₅ alkyl)COO—(C₁₋₅ alkyl), preferably selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(O)(C₁₋₅ alkyl), S(O)₂(C₁₋₅ alkyl), S(O)(NH)(C₁₋₅ alkyl), S(O)(N(C₁₋₅ alkyl))(C₁₋₅ alkyl), —N═S(O)(C₁₋₅ alkyl)(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), COO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), NHCOO(C₁₋₅ alkyl), N(C₁₋₅ alkyl)COO—(C₁₋₅ alkyl), —P(O)(C₁₋₅)(C₁₋₅ alkyl), —P(a)((C₁₋₅ alkyl))(O(C₁₅ alkyl)), —P(O)(O(C₁₋₅ alkyl))(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl)(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)S(O)(C₁₋₅ alkyl), —(C₁₋₅ alkylene)S(O)₂(C₁₋₅ alkyl), —(C₁₋₅ alkylene)S(O)(NH)(C₁₋₅ alkyl), —(C₁₋₅ alkylene)S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)((C₁₋₅ alkyl))(C₁₋₅ alkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ (C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkyl)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ haloalkyl)CONH₂, —(C₁₋₅ alkylene)N(C₁₋₅ alkyl)CONH—(C₁₋₅ alkyl), —(C₁₋₅ alkylene)N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)NHCOO(C₁₋₅ alkyl), and —(C₁₋₅ alkylene)N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl), —N—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from, halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl). More preferably, —Y_{C2}—R_{C2} is azacyclohexenyl, optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(O)(C₁₋₅ alkyl), S(O)₂(C₁₋₅ alkyl), S(O)(NH)(C₁₋₅ alkyl), S(O)(N(C₁₋₅ alkyl))(C₁₋₅ alkyl), —N═S(O)(C₁₋₅ alkyl)(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, COO(C₁₋₅ alkyl), —COO(C₁₋₅ haloalkyl), —COO-cycloalkyl, CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), NHCOO(C₁₋₅ alkyl), N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)S(O)(C₁₋₅ alkyl), —(C₁₋₅ alkylene)S(O)₂(C₁₋₅ alkyl), —(C₁₋₅ alkylene)S(O)(NH)(C₁₋₅ alkyl), —(C₁₋₅ alkylene)S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), —(C₁₋₅ alkylene)N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)NHCOO(C₁₋₅ alkyl), and —(C₁₋₅ alkylene)N(C₁₋₅ alkyl)COO—(C₁₋₅ alkyl), preferably selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(O)(C₁₋₅ alkyl), S(O)₂(C₁₋₅ alkyl), S(O)(NH)(C₁₋₅ alkyl), S(O)(N(C₁₋₅ alkyl))(C₁₋₅ alkyl), —N═S(O)(C₁₋₅ alkyl)(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), COO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), NHCOO(C₁₋₅ alkyl), N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)S(O)(C₁₋₅ alkyl), —(C₁₋₅ alkylene)S(O)₂(C₁₋₅ alkyl), —(C₁₋₅ alkylene)S(O)(NH)(C₁₋₅ alkyl), —(C₁₋₅ alkylene)S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), —(C₁₋₅ alkylene)N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)NHCOO(C₁₋₅ alkyl), and —(C₁₋₅ alkylene)N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl). Even more preferably, —Y_{C2}—R_{C2} is azacyclohexenyl substituted (preferably N-substituted) with —CON(C₁₋₅ alkyl)(C₁₋₅ alkyl), preferably with —CON(CH₃)₂. Preferably, azacyclohexenyl as referred to herein is 1,2,3,6-tetrahydropyridinyl.

In another preferred embodiment, if —Y_{C2}—R_{C2} is heterocycloalkyl, —Y_{C2}—R_{C2} is 2-oxaspiro[3.5]non-6-en-7-yl, 2-oxaspiro[3.5]non-7-yl, 2-oxa-8-azaspiro[4.5]dec-8-yl, 9-oxa-3-azaspiro[5.5]undec-3-yl, 2-oxa-6-azaspiro[3.4]oct-6-yl, 1-oxa-7-azaspiro[3.5]non-7-yl, 1-oxa-8-azaspiro[4.5]dec-8-yl, 6-oxa-2-azaspiro[3.3]hept-2-yl, 2,8-diazaspiro[4.5]dec-8-yl, 7-oxa-3-azabicyclo[3.3.0]oct-3-yl, 8-oxa-3-azabicyclo[4.3.0]non-3-yl, 2-oxa-6-azaspiro[3.5]non-6-yl, 7-oxo-3,6,8-triazabicyclo[4.3.0]non-3-yl, 3-pyrrolino[3,4-c]pyrazol-2-yl, 3,6-diazabicyclo[3.1.1]hept-3-yl, or 2,7-diazaspiro[3.5]non-7-yl.

In one specific embodiment, —Y_{C2}—R_{C2} is selected from:

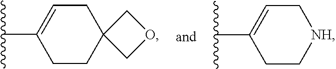 [SMILES: *C1=CCC2(CC1)COC2 ; *C1=CCNCC1]

preferably —Y_{C2}—R_{C2} is

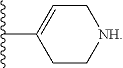 [SMILES: *C1=CCNCC1]

In one specific embodiment, —Y_{C2}—R_{C2} is selected from:

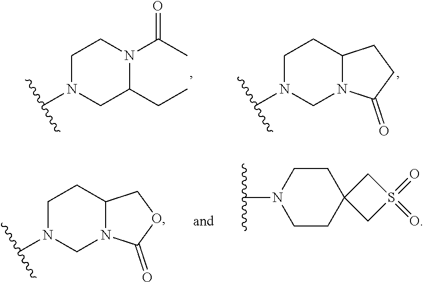

In one specific embodiment, —Y_{C2}—R_{C2} is selected from:

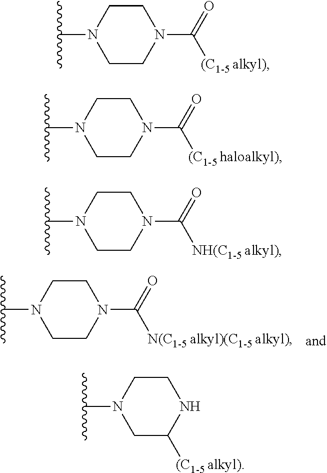

X₄ is N or C—R_{C4}.
R_{C4} is selected from hydrogen, halo, C₁₋₆ alkyl, C₂₋₆ alkynyl, —O(C₁₋₆ alkyl), —S(C₁₋₆ alkyl), —NH(C₁₋₆ alkyl), —N(C₁₋₆ alkyl)(C₁₋₆ alkyl), —CO(C₁₋₆ alkyl), C₁₋₆ haloalkyl, —O(C₁₋₆ haloalkyl), —S(C₁₋₆ haloalkyl), —NH(C₁₋₆ haloalkyl), —N(C₁₋₆ haloalkyl)₂, —CO(C₁₋₆ haloalkyl), —(C₀₋₃ alkylene)cycloalkyl, —O—(C₀₋₃ alkylene)-cycloalkyl, —CO—(C₀₋₃ alkylene)-cycloalkyl, —(C₀₋₃ alkylene)cycloalkenyl, —O—(C₀₋₃ alkylene)-cycloalkenyl, —CO—(C₀₋₃ alkylene)-cycloalkenyl, —(C₀₋₃ alkylene)-heterocycloalkyl, —O—(C₀₋₃ alkylene)-heterocycloalkyl, —CO—(C₀₋₃ alkylene)-heterocycloalkyl, —(C₀₋₃ alkylene)-heterocycloalkenyl, —O—(C₀₋₃ alkylene)-heterocycloalkenyl, —CO—(C₀₋₃ alkylene)-heterocycloalkenyl, —(C₀₋₃ alkylene)-aryl, —O—(C₀₋₃ alkylene)-aryl, —CO—(C₀₋₃ alkylene)-aryl, —(C₀₋₃ alkylene)-heteroaryl, —O—(C₀₋₃ alkylene)-heteroaryl and —CO—(C₀₋₃ alkylene)-heteroaryl, preferably selected from hydrogen, halo, C₁₋₆ alkyl, C₂₋₆ alkynyl, —O(C₁₋₆ alkyl), —S(C₁₋₆ alkyl), —NH(C₁₋₆ alkyl), —N(C₁₋₆ alkyl)(C₁₋₆ alkyl), —CO(C₁₋₆ alkyl), C₁₋₆ haloalkyl, —O(C₁₋₆ haloalkyl), —S(C₁₋₆ haloalkyl), —NH(C₁₋₆ haloalkyl), —N(C₁₋₆ haloalkyl)₂, —CO(C₁₋₆ haloalkyl), —(C₀₋₃ alkylene)cycloalkyl, —O—(C₀₋₃ alkylene)-cycloalkyl, —CO—(C₀₋₃ alkylene)-cycloalkyl, —(C₀₋₃ alkylene)-heterocycloalkyl, —O—(C₀₋₃ alkylene)-heterocycloalkyl, —CO—(C₀₋₃ alkylene)-heterocycloalkyl, —(C₀₋₃ alkylene)-aryl, —O—(C₀₋₃ alkylene)-aryl, —CO—(C₀₋₃ alkylene)-aryl, —(C₀₋₃ alkylene)-heteroaryl, —O—(C₀₋₃ alkylene)-heteroaryl and —CO—(C₀₋₃ alkylene)-heteroaryl. Said alkyl or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), preferably selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably said alkyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), preferably selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

Preferably R_{C4} is selected from hydrogen, halo, C₁₋₅ alkyl, C₂₋₆ alkynyl, —O(C₁₋₆ alkyl), —S(C₁₋₆ alkyl), —NH(C₁₋₆ alkyl), C₁₋₆ haloalkyl, —(C₀₋₃ alkylene)-cycloalkyl, —(C₀₋₃ alkylene)-cycloalkenyl, —(C₀₋₃ alkylene)-heterocycloalkyl, —(C₀₋₃ alkylene)-heterocycloalkenyl, —(C₀₋₃ alkylene)-aryl and —(C₀₋₃ alkylene)-heteroaryl, preferably selected from hydrogen, halo, C₁₋₆ alkyl, C₂₋₆ alkynyl, —O(C₁₋₆ alkyl), —S(C₁₋₆ alkyl), —NH(C₁₋₆ alkyl), C₁₋₆ haloalkyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl. Said alkyl or alkynyl (preferably said alkyl) is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), preferably selected from halogen, —CN, —OH, —O(C₁₋₅ alkyl), —O(C₁₋₅ haloalkyl), —SH, —S(C₁₋₅ alkyl), —O(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ haloalkyl), —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

Further preferably, R_{C4} is selected from hydrogen, halo, C₁₋₅ alkyl, C₂₋₆ alkynyl, —O—C₁₋₅ alkyl, —S—C₁₋₆ alkyl, —NH—C₁₋₆ alkyl, and C₁₋₆ haloalkyl, more preferably R_{C4} is selected from hydrogen, halo, C₁₋₂ alkyl, and C₂₋₃ alkynyl, even more preferably R_{C4} is selected from hydrogen, halo, and C₁₋₂ alkyl, even more preferably R_{C4} is hydrogen or halo.

In an alternative preferred embodiment, R_{C4} is selected from —(C₀₋₃ alkylene)-cycloalkyl, —(C₀₋₃ alkylene)-cycloalkenyl, —(C₀₋₃ alkylene)-heterocycloalkyl, —(C₀₋₃ alkylene)-heterocycloalkenyl, —(C₀₋₃ alkylene)-aryl and —(C₀₋₃ alkylene)-heteroaryl, preferably selected from —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl. Said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). More preferably, R_{C4} is selected from —(C₀₋₃ alkylene)-cycloalkyl, —(C₀₋₃ alkylene)-heterocycloalkyl, and —(C₀₋₃ alkylene)-heteroaryl, preferably from cycloalkyl, heterocycloalkyl, and heteroaryl. Said cycloalkyl, heterocycloalkyl, or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —NH(C₁₋₅ haloalkyl)(C₁₋₅ alkyl), —CONH₂, —CONH(C₁₋₅ alkyl), and —CON(C₁₋₅ alkyl)(C₁₋₅ alkyl). Even more preferably, R_{C4} is selected from heterocycloalkyl, and heteroaryl. Said heterocycloalkyl, or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —NH(C₁₋₅ haloalkyl)(C₁₋₅ alkyl), —CONH₂, —CONH(C₁₋₅ alkyl), and —CON(C₁₋₅ alkyl)(C₁₋₅ alkyl).

In an alternative preferred embodiment, R_{C4} is selected from —CH₂-cycloalkyl, —CH₂-cycloalkenyl, —CH₂-heterocycloalkyl, —CH₂-heterocycloalkenyl, —CH₂-aryl and —CH₂-heteroaryl, preferably selected from —CH₂-cycloalkyl, —CH₂-heterocycloalkyl, —CH₂-aryl and —CH₂-heteroaryl. Said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). More preferably R_{C4} is selected from —CH₂-heterocycloalkyl, and —CH₂-heteroaryl. Said heterocycloalkyl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

If R_{C4} is heteroaryl, R_{C4} is preferably imidazolyl, pyridazinyl, thiazolyl, pyridinyl, pyrimidinyl, pyrazinyl, or indazolyl, wherein heteroaryl may be optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

If R_{C4} is heterocycloalkyl, R_{C4} is preferably morpholinyl, 1,1-dioxothiomorpholinyl, azetinyl, pyrrolidinyl, piperidinyl, 6-oxo-1,6-dihydropyridinyl, or piperazinyl, wherein heterocycloalkyl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl, preferably selected from halogen, —CN, —OH, C₁₋₅ alkyl, C₁₋₅ haloalkyl, —(C₁₋₅ alkyl), —O(C₁₋₅ haloalkyl), —SH, —S(C₁₋₅ alkyl, —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). More preferably, R_{C4} is piperazinyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —C—(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₆ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Even more preferably, R_{C4} is piperazinyl (preferably N-piperazinyl) optionally substituted (preferably N-substituted) with —CONH₂, —CONH(C₁₋₅ alkyl), and —CON(C₁₋₅ alkyl)(C₁₋₅ alkyl). Most preferably, R_{C4} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.

If R_{C4} is heterocycloalkenyl, R_{C4} is oxacyclohexenyl or azacyclohexenyl, preferably R_{C4} is azacyclohexenyl, wherein heterocycloalkenyl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl)-, —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-(C₁₋₅ alkyl), —(C₁₋₅ alkylene)-(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl, preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

In an alternative embodiment wherein R_{C4} is heterocycloalkyl, R_{C4} is preferably 2-oxaspiro[3.5]non-6-en-7-yl, 2-oxaspiro[3.5]non-7-yl, 2-oxa-8-azaspiro[4.5]dec-8-yl, 9-oxa-3-azaspiro[5.5]undec-3-yl, 2-oxa-6-azaspiro[3.4]oct-6-yl, 1-oxa-7-azaspiro[3.5]non-7-yl, 1-oxa-8-azaspiro[4.5]dec-8-yl, 6-oxa-2-azaspiro[3.3]hept-2-yl, 2,8-diazaspiro[4.5]dec-8-yl, 7-oxa-3-azabicyclo[3.3.0]oct-3-yl, 8-oxa-3-azabicyclo[4.3.0]non-3-yl, 2-oxa-6-azaspiro[3.5]non-6-yl, 7-oxo-3,6,8-triazabicyclo[4.3.0]non-3-yl, 3-pyrrolino[3,4-c]pyrazol-2-yl, 3,6-diazabicyclo[3.1.1]hept-3-yl, or 2,7-diazaspiro[3.5]non-7-yl.

Preferably, if X₂ comprises cycloalkyl, heterocycloalkyl, aryl or heteroaryl, X₄ is C—R_{C4} wherein R_{C4} is selected from hydrogen, halo, C₁₋₆ alkyl, —O(C₁₋₆ alkyl), —S(C₁₋₆ alkyl), —NH(C₁₋₆ alkyl), and C₁₋₆ haloalkyl.

More preferably, if X₂ comprises cycloalkyl, heterocycloalkyl, aryl or heteroaryl, X₄ is C—R_{C4} wherein R_{C4} is selected from hydrogen, and halo.

Further preferably, if X₄ comprises cycloalkyl, heterocycloalkyl, aryl or heteroaryl, X₂ does not comprise any of the groups cycloalkyl, heterocycloalkyl, aryl and heteroaryl.

Further preferably, if X₂ comprises cycloalkyl, heterocycloalkyl, aryl or heteroaryl and X₄ comprises cycloalkyl, heterocycloalkyl, aryl or heteroaryl, then together R_{C4} and —Y_{C2}—R_{C2} include not more than 12 non-hydrogen atoms, preferably not more than 10 non-hydrogen atoms.
X₅ is N or C—R_{C5}. Preferably not more than one of X₄ and X₅ is N. In certain preferred embodiments, X₄ is N and X₅ is C—R_{C5}, preferably X₄ is N and X₅ is CH. In certain preferred embodiments, X₄ is C—R_{C4} and X₅ is N, preferably X₄ is CH and X₅ is N. In certain preferred embodiments, X₄ is C—R_{C4} and X₅ is C—R_{C5}. In certain preferred embodiments X₄ is CH and X₅ is CH.
R_{C5} is selected from hydrogen, halo, C₁₋₆ alkyl, —O(C₁₋₆ alkyl), —S(C₁₋₆ alkyl), —NH(C₁₋₆ alkyl), —N(C₁₋₆ alkyl)C₁₋₆ alkyl and C₁₋₆ haloalkyl. Preferably, R_{C5} is selected from hydrogen, halo, C₁₋₃ alkyl, —O(C₁₋₃ alkyl), —S(C₁₋₃ alkyl), —NH(C₁₋₃ alkyl), and C₁₋₃ haloalkyl. More preferably, R_{C5} is selected from hydrogen, halo, C₁₋₃ alkyl, and C₁₋₃ haloalkyl.
R₄ is Y_{R5}—R_{R5}.
Y_{R5} is selected from a covalent bond, C₁₋₄ alkylene, C₂₋₄ alkenylene, and C₂₋₄ alkynylene, wherein said alkylene, said alkenylene and said alkynylene are each optionally substituted with one or more groups independently selected from halogen, —CN, —OH, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), and —N(C₁₋₅ haloalkyl)(C₁₋₅ alkyl), preferably selected from halogen, —CN, —OH, —O(C₁₋₅ alkyl), —O(C₁₋₅ haloalkyl), —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH(C₁₋₅ alkyl), —NH(C₁₋₅ haloalkyl), —N(C₁₋₅ alkyl)(C₁₋₅ alkyl), and —N(C₁₋₅ haloalkyl)(C₁₋₅ alkyl), more preferably selected from halogen, —CN, —OH, —O(C₁₋₅ alkyl), —O(C₁₋₅ haloalkyl), —S(C₁₋₅ alkyl), —S(C₁₋₅ haloalkyl), —NH(C₁₋₅ alkyl), —NH(C₁₋₅ haloalkyl), —N(C₁₋₅ alkyl)(C₁₋₅ alkyl), and —N(C₁₋₅ haloalkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, —COO—, S—, —SO—, and SO₂—, preferably selected from —O—, NH—, N(C₁₋₅ alkyl)-, CO—, S—, —SO—, and SO₂—. Preferably, Y_{R5} is selected from a covalent bond, C₁₋₂ alkylene, —CO—(C₁₋₂ alkylene)-, —(C₁₋₂ alkylene)-CO—, —CONH—(C₁₋₂ alkylene)-, —(C₁₋₂ alkylene)-CONH—, —NHCO—(C₁₋₂ alkylene)-, —(C₁₋₂ alkylene)-NHCO—, —NH—(C₁₋₂ alkylene)-, —(C₁₋₂ alkylene)-NH—, —O—(C₁₋₂ alkylene)-, —(C₁₋₂ alkylene)-O—, SO₂—(C₁₋₂ alkylene)-, —(C₁₋₂ alkylene)SO₂—, —CONH—, CON(C₁₋₅ alkyl)-, —NHCO—, —N(C₁₋₅ alkyl)CO—, —NH—, —O—, —CO—, —COO— and —SO₂—. C₁₋₂ alkylene is herein preferably a —CH₂— group.
R_{R5} is selected from C₁₋₁₂ alkyl, C₂₋₁₂ alkenyl, C₂₋₁₂ alkynyl, cycloalkyl, cycloalkenyl, heterocycloalkyl, heterocycloalkenyl, aryl, and heteroaryl, preferably selected from C₁₋₁₂ alkyl, C₂₋₁₂ alkenyl, C₂₋₁₂ alkynyl, cycloalkyl, heterocycloalkyl, aryl, and heteroaryl. Preferably R_{R5} is selected from cycloalkyl, heterocycloalkyl, aryl, and heteroaryl. More preferably, R_{R5} is selected from heterocycloalkyl, aryl, and heteroaryl. Even more preferably, R_{R5} is selected from aryl and heteroaryl. Most preferably, R_{R5} is heteroaryl. Said alkyl, alkenyl, or alkynyl is optionally substituted with one or more groups independently selected from halogen, —CN, —OH, —O(C₁₋₅ alkyl), —O(C₁₋₅ haloalkyl), —SH, —S(C₁₋₅ alkyl), —S(C₁₋₅ haloalkyl), NH₂₁, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl). Said cycloalkyl, cycloalkenyl, heterocycloalkyl, heterocycloalkenyl, aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

Preferably, Y_{R5} is selected from a covalent bond, C₁₋₂ alkylene, —CO—(C₁₋₂ alkylene)-, —(C₁₋₂ alkylene)-CO—, —CONH—(C₁₋₂ alkylene)-, —(C₁₋₂ alkylene)-CONH—, —NHCO—(C₁₋₂ alkylene)-, —(C₁₋₂ alkylene)-NHCO—, —NH—(C₁₋₂ alkylene)-, —(C₁₋₂ alkylene)-NH—, —O—(C₁₋₂ alkylene)-, —(C₁₋₂ alkylene)-O—, SO₂—(C₁₋₂ alkylene), —(C₁₋₂ alkylene)SO₂—, —CONH—, —NHCO—, —NH—, —O—, —CO— and SO₂—. Thus, preferably, R₄ is selected from —(C₀₋₂ alkylene)-cycloalkyl, —CO—(C₀₋₂ alkylene)-cycloalkyl, —(C₀₋₂ alkylene)-CO-cycloalkyl, —CONH—(C₀₋₂ alkylene)-cycloalkyl, —(C₀₋₂ alkylene)-CONH-cycloalkyl, —NHCO—(C₀₋₂ alkylene)-cycloalkyl, —(C₀₋₂ alkylene)-NHCO-cycloalkyl, —NH—(C₀₋₂ alkylene)-cycloalkyl, —(C₀₋₂ alkylene)-NH-cycloalkyl, —O—(C₀₋₂ alkylene)-cycloalkyl, —(C₀₋₂ alkylene)-O-cycloalkyl, SO₂—(C₀₋₂ alkylene)-cycloalkyl, —(C₀₋₂ alkylene)SO₂-cycloalkyl, —CONH-cycloalkyl, —NHCO-cycloalkyl, —NH-cycloalkyl, —O-cycloalkyl, —CO-cycloalkyl, SO₂-cycloalkyl, —(C₀₋₂ alkylene)-cycloalkenyl, —CO—(C₀₋₂ alkylene)-cycloalkenyl, —(C₀₋₂ alkylene)-CO-cycloalkenyl, —CONH—(C₀₋₂ alkylene)-cycloalkenyl, —(C₀₋₂ alkylene)-CONH-cycloalkenyl, —NHCO—(C₀₋₂ alkylene)-cycloalkenyl, —(C₀₋₂ alkylene)-NHCO-cycloalkenyl, —NH—(C₀₋₂ alkylene)-cycloalkenyl, —(C₀₋₂ alkylene)-NH-cycloalkenyl, —O—(C₀₋₂ alkylene)-cycloalkenyl, —(C₀₋₂ alkylene)-O-cycloalkenyl, SO₂—(C₀₋₂ alkylene)-cycloalkenyl, —(C₀₋₂ alkylene)SO₂-cycloalkenyl, —CONH-cycloalkenyl, —NHCO-cycloalkenyl, —NH— cycloalkenyl, —O-cycloalkenyl, —CO-cycloalkenyl, SO₂-cycloalkenyl, —(C₀₋₂ alkylene)-heterocycloalkyl, —CO—(C₀₋₂ alkylene)-heterocycloalkyl, —(C₀₋₂ alkylene)-CO-heterocycloalkyl, —CONH—(C₀₋₂ alkylene)-heterocycloalkyl, —(C₀₋₂ alkylene)-CONH-heterocycloalkyl, —NHCO—(C₀₋₂ alkylene)-heterocycloalkyl, —(C₀₋₂ alkylene)-NHCO-heterocycloalkyl, —NH—(C₀₋₂ alkylene)-heterocycloalkyl, —(C₀₋₂ alkylene)-NH-heterocycloalkyl, —O—(C₀₋₂ alkylene)-heterocycloalkyl, —(C₀₋₂ alkylene)-O-heterocycloalkyl, SO₂—(C₀₋₂ alkylene)-heterocycloalkyl, —(C₀₋₂ alkylene)SO₂-heterocycloalkyl, —CONH-heterocycloalkyl, —NHCO— heterocycloalkyl, —NH-heterocycloalkyl, —O-heterocycloalkyl, —CO-heterocycloalkyl, SO₂-heterocycloalkyl, —(C₀₋₂ alkylene)-heterocycloalkenyl, —CO—(C₀₋₂ alkylene)-heterocycloalkenyl, —(C₀₋₂ alkylene)-CO-heterocycloalkenyl, —CONH—(C₀₋₂ alkylene)-heterocycloalkenyl, —(C₀₋₂ alkylene)-CONH-heterocycloalkenyl, —NHCO—(C₀₋₂ alkylene)-heterocycloalkenyl, —(C₀₋₂ alkylene)-NHCO-heterocycloalkenyl, —NH—(C₀₋₂ alkylene)-heterocycloalkenyl, —(C₀₋₂ alkylene)-NH-heterocycloalkenyl, —O—(C₀₋₂ alkylene)-heterocycloalkenyl, —(C₀₋₂ alkylene)-O-heterocycloalkenyl, SO₂—(C₀₋₂ alkylene)-heterocycloalkenyl, —(C₀₋₂ alkylene)SO₂-heterocycloalkenyl, —CONH-heterocycloalkenyl, —NHCO— heterocycloalkenyl, —NH-heterocycloalkenyl, —O-heterocycloalkenyl, —CO-heterocycloalkenyl, SO₂-heterocycloalkenyl, —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, SO₂—(C₀₋₂ alkylene)-aryl, —(C₀₋₂ alkylene)SO₂-aryl, —CONH-aryl, —NHCO-aryl, —NH-aryl, —O-aryl, —CO-aryl, SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, SO₂—(C₀₋₂ alkylene)-heteroaryl, —(C₀₋₂ alkylene)SO₂-heteroaryl, —CONH-heteroaryl, —NHCO-heteroaryl, —NH-heteroaryl, —O-heteroaryl, —CO-heteroaryl, and SO₂-heteroaryl, preferably selected from —(C₀₋₂ alkylene)-cycloalkyl, —CO—(C₀₋₂ alkylene)-cycloalkyl, —(C₀₋₂ alkylene)-CO-cycloalkyl, —CONH—(C₀₋₂ alkylene)-cycloalkyl, —(C₀₋₂ alkylene)-CONH-cycloalkyl, —NHCO—(C₀₋₂ alkylene)-cycloalkyl, —(C₀₋₂ alkylene)-NHCO-cycloalkyl, —NH—(C₀₋₂ alkylene)-cycloalkyl, —(C₀₋₂ alkylene)-NH-cycloalkyl, —O—(C₀₋₂ alkylene)-cycloalkyl, —(C₀₋₂ alkylene)-O-cycloalkyl, SO₂—(C₀₋₂ alkylene)-cycloalkyl, —(C₀₋₂ alkylene)SO₂-cycloalkyl, —CONH-cycloalkyl, —NHCO-cycloalkyl, —NH-cycloalkyl, —O-cycloalkyl, —CO— cycloalkyl, SO₂-cycloalkyl, —(C₀₋₂ alkylene)-heterocycloalkyl, —CO—(C₀₋₂ alkylene)-heterocycloalkyl, —(C₀₋₂ alkylene)-CO-heterocycloalkyl, —CONH—(C₀₋₂ alkylene)-heterocycloalkyl, —(C₀₋₂ alkylene)-CONH-heterocycloalkyl, —NHCO—(C₀₋₂ alkylene)-heterocycloalkyl, —(C₀₋₂ alkylene)-NHCO-heterocycloalkyl, —NH—(C₀₋₂ alkylene)-heterocycloalkyl, —(C₀₋₂ alkylene)-NH-heterocycloalkyl, —O—(C₀₋₂ alkylene)-heterocycloalkyl, —(C₀₋₂ alkylene)-O-heterocycloalkyl, SO₂—(C₀₋₂ alkylene)-heterocycloalkyl, —(C₀₋₂ alkylene)SO₂-heterocycloalkyl, —CONH-heterocycloalkyl, —NHCO-heterocycloalkyl, —NH-heterocycloalkyl, —O— heterocycloalkyl, —CO-heterocycloalkyl, SO₂-heterocycloalkyl, —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, SO₂—(C₀₋₂ alkylene)-aryl, —(C₀₋₂ alkylene)SO₂-aryl, —CONH-aryl, —NHCO-aryl, —NH-aryl, —O-aryl, —CO-aryl, SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, SO₂—(C₀₋₂ alkylene)-heteroaryl, —(C₀₋₂ alkylene)SO₂-heteroaryl, —CONH-heteroaryl, —NHCO-heteroaryl, —NH-heteroaryl, —O-heteroaryl, —CO-heteroaryl, and SO₂-heteroaryl. Said cycloalkyl, cycloalkenyl, heterocycloalkyl, heterocycloalkenyl, aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl). More preferably, R₄ is selected from —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, SO₂—(C₀₋₂ alkylene)-aryl, —(C₀₋₂ alkylene)SO₂-aryl, —CONH-aryl, —NHCO-aryl, —NH-aryl, —O-aryl, —CO-aryl, SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, SO₂—(C₀₋₂ alkylene)-heteroaryl, —(C₀₋₂ alkylene)SO₂-heteroaryl, —CONH-heteroaryl, —NHCO-heteroaryl, —NH-heteroaryl, —O-heteroaryl, —CO-heteroaryl, and SO₂-heteroaryl, wherein said aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl).

In certain embodiments, R₄ is selected from —(C₀₋₂ alkylene)-CO-cycloalkyl, preferably —CO— cyclohexyl, and —(C₀₋₂ alkylene)-CO-aryl, preferably —CO-phenyl.

In certain embodiments, R₄ is selected from —COO—(C₁₋₅ alkyl) or —CONH—(C₁₋₅ alkyl).

In one specific embodiment, R₄ is selected from:

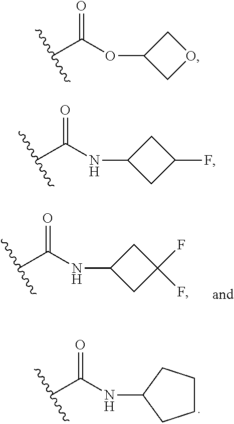 [SMILES: *C(=O)OC1COC1 ; *C(=O)NC1CC(F)C1 ; *C(=O)NC1CC(F)(F)C1 ; *C(=O)NC1CCCC1]

Preferably, Y_{R5} is a covalent bond. Thus, R₄ is preferably selected from C₁₋₁₂ alkyl, C₂₋₁₂ alkenyl, C₂₋₁₂ alkynyl, cycloalkyl, cycloalkenyl, heterocycloalkyl, heterocycloalkenyl, aryl, and heteroaryl, more preferably selected from C₁₋₁₂ alkyl, C₂₋₁₂ alkenyl, C₂₋₁₂ alkynyl, cycloalkyl, heterocycloalkyl, aryl, and heteroaryl. More preferably, R₄ is selected from cycloalkyl, cycloalkenyl, heterocycloalkyl, heterocycloalkenyl, aryl, and heteroaryl. More preferably, R₄ is selected from cycloalkyl, heterocycloalkyl, aryl, and heteroaryl. Even more preferably, R₄ is selected from aryl, and heteroaryl. Most preferably, R₄ is heteroaryl. Said alkyl, alkenyl, or alkynyl is optionally substituted with one or more groups independently selected from halogen, —CN, —OH, —O(C₁₋₅ alkyl), —O(C₁₋₅ haloalkyl), SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Said cycloalkyl, cycloalkenyl, heterocycloalkyl, heterocycloalkenyl, aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

Preferably, R₄ is a five membered heteroaryl, optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). The said five membered heteroaryl is preferably selected from imidazolyl, isoxazolyl, pyrazolyl, 1,2,3-triazolyl, 1,2,4-triazolyl, thiazolyl, 1,2,4-oxadiazolyl, 1,3,4-oxadiazolyl, 1,2,4-thiadiazolyl, or 1,3,4-thiadiazolyl. More preferably, said five membered heteroaryl is 1,2,4-thiadiazolyl, optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CONH₂, —CONH(C₁₋₅ alkyl), and —CON(C₁₋₅ alkyl)(C₁₋₅ alkyl), preferably optionally substituted with C₁₋₅ alkyl, C₁₋₅ haloalkyl, —O(C₁₋₅ alkyl), —SH, —S(C₁₋₅ alkyl), more preferably optionally substituted with C₁₋₅ alkyl, C₁₋₅ haloalkyl, even more preferably optionally substituted with C₁₋₅ haloalkyl, preferably selected from —CH₂F, —CHF₂ and CF₃, most preferably optionally substituted with —CHF₂.

In one specific embodiment, R₄ is

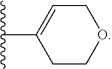 [SMILES: *C1=CCOCC1]

Preferably, the present invention relates to a compound of formula (I) wherein W is —NHS(O)₂—. Thus, in another embodiment, the present invention relates to a compound of formula (Ia):

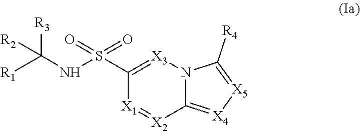

R₁, R₂, R₃, R₄, X₁, X₂, X₃, X₄, and X₅ in the compound of formula (Ia) are as defined hereinabove for the compound of formula (I).

In a preferred embodiment, R₂ and R₃ together with the carbon atom to which they are attached form cyclopropyl. Thus, preferably, the compound of formula (Ia) is a compound of formula (Ib):

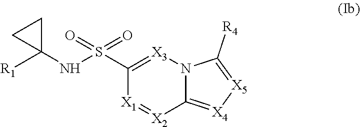

R₁, R₄, X₁, X₂, X₃, X₄, and X₅ in the compound of formula (Ib) are as defined hereinabove for the compound of formula (I).

R₁ is selected from the group consisting of hydrogen, chloro, fluoro, cyano, formyl, (C₁₋₂)alkyl, (C₂)alkenyl, (C₂)alkynyl (C₁₋₂)haloalkyl, —(C₁₋₂ alkylene)-OH and —(C₁₋₂ alkylene)-O—(C₁₋₂ alkyl). Preferably, R₁ is selected from the group consisting of hydrogen, chloro, fluoro, cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)haloalkyl and (C₁₋₂)alkyl, preferably cyano, fluoromethyl and methyl. More preferably, R₁ is cyano. Thus, in a preferred embodiment, the compound of formula (Ib) is a compound of formula (Ic):

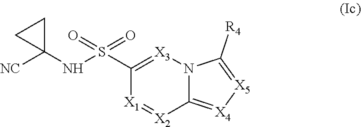

R₄, X₁, X₂, X₃, X₄, and X₅ in the compound of formula (Ic) are as defined hereinabove for the compound of formula (I).

Within the scope of the present invention, the compound of formula (I) or the compound of formula (Ia) or the compound of formula (Ib) wherein R₁ is methyl is also encompassed. In certain preferred embodiments of the present invention, R₁ is methyl. Within the scope of the present invention, the compound of formula (I) or the compound of formula (Ia) or the compound of formula (Ib) wherein R₁ is fluoromethyl is also encompassed. In certain preferred embodiments of the present invention, R₁ is fluoromethyl.

Preferably, within the scope of the present invention, X₁ and X₃ are each CH. Thus, preferably the compound of formula (I) of the present invention is a compound of formula (Id):

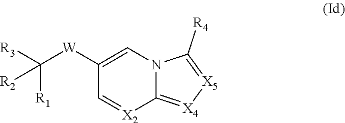

W, R₁, R₂, R₃, R₄, X₂, X₄, and X₅ in the compound of formula (Id) are as defined hereinabove for the compound of formula (I).

Preferably, the present invention relates to a compound of formula (I) wherein W is —NHS(O)₂—. Thus, in another embodiment, the compound of formula (Id) of the present invention is a compound of formula (Ie):

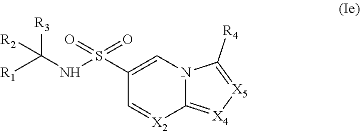

R₁, R₂, R₃, R₄, X₂, X₄, and X₅ in the compound of formula (Ie) are as defined hereinabove for the compound of formula (I).

In a preferred embodiment, R₂ and R₃ together with the carbon atom to which they are attached form cyclopropyl. Thus, preferably, the compound of formula (Ie) of the present invention is a compound of formula (If):

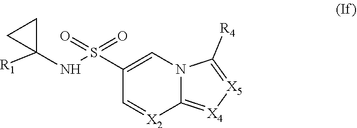

R₁, R₄, X₂, X₄, and X₅ in the compound of formula (If) is defined as defined hereinabove for the compound of formula (I) of the present invention.

Within the scope of the present invention, R₁ is preferably selected from the group consisting of hydrogen, chloro, fluoro, cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)haloalkyl and (C₁₋₂)alkyl, preferably cyano, fluoromethyl and methyl. More preferably, R₁ is cyano.

Thus preferably, the compound of formula (If) of the present invention is a compound of formula (Ig):

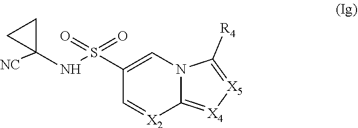

R₄, X₂, X₄, and X₅ in the compound of formula (Ig) are as defined hereinabove for the compound of formula (I) of the present invention.

Within the scope of the present invention, the compound of formula (Id) or the compound of formula (Ie) or the compound of formula (If) wherein R₁ is methyl is also encompassed within the present invention. In certain preferred embodiments of the present invention, R₁ is methyl. Within the scope of the present invention, the compound of formula (Id) or the compound of formula (Ie) or the compound of formula (If) wherein R₁ is fluoromethyl is also encompassed. In certain preferred embodiments of the present invention, R₁ is fluoromethyl.

As encompassed by the present invention, X₄ is N or C—R_{C4} and X₅ is N or C—R_{C5}. Furthermore, as defined hereinabove, preferably not more than one of X₄ and X₅ is N. In certain embodiments of the present invention, X₅ is N. Thus, if X₅ is N, preferably X₄ is C—R_{C4}. Thus, in certain preferred embodiments the compound of formula (I) is a compound of formula (Ih):

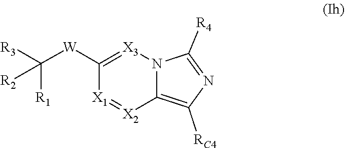

W, R_{C4}, R₁, R₂, R₃, R₄, X₁, X₂, and X₃ in the compound of formula (Ih) are as defined hereinabove for the compound of formula (I).

Preferably, within the scope of the present invention W is —NHS(O)₂—. Thus, preferably, the compound of formula (I) or the compound of formula (Ia) or the compound of formula (Ih) is a compound of formula (Ii):

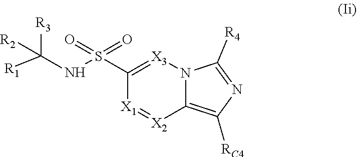

R_{C4}, R₁, R₂, R₃, R₄, X₁, X₂, and X₃ in the compound of formula (Ii) are as defined hereinabove for the compound of formula (I).

In a preferred embodiment, R₂ and R₃ together with the carbon atom to which they are attached form cyclopropyl. Thus, preferably, the compound of formula (Ii) of the present invention is a compound of formula (Ij):

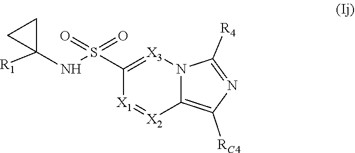

R_{C4}, R₁, R₄, X₁, X₂, and X₃ in the compound of formula (Ij) are as defined hereinabove for the compound of formula (I).

Within the scope of the present invention, preferably R₁ is hydrogen, chloro, fluoro, cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)haloalkyl and (C₁₋₂)alkyl, preferably cyano, fluoromethyl and methyl. More preferably, R₁ is cyano.

Thus, preferably within the scope of the present invention the compound of formula (I) or the compound of formula (Ib) or the compound of formula (Ic) or the compound of formula (Ih) or the compound of formula (Ii) or the compound of formula (Ij) of the present invention is a compound of formula (Ik):

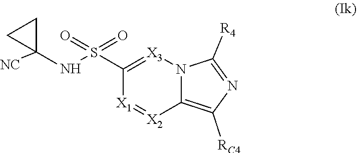

R_{C4}, R₄, X₁, X₂, and X₃ in the compound of formula (Ik) are as defined hereinabove for the compound of formula (I).

In one embodiment of the compound of formula (I) of the present invention, X₁ and X₃ are each CH. Thus, preferably the compound of formula (I) of the present invention is a compound of formula (IL):

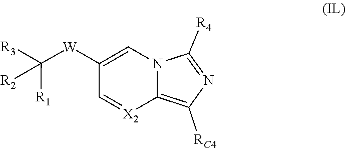

W, R_{C4}, R₁, R₂, R₃, R₄, and X₂ in the compound of formula (IL) are as defined hereinabove for the compound of formula (I).

Preferably, within the scope of the present invention W is —NHS(O)₂—. Thus, preferably, the compound of formula (IL) of the present invention is a compound of formula (Im):

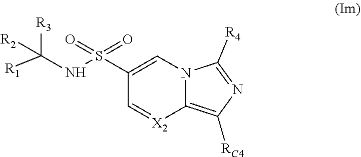

R_{C4}, R₁, R₂, R₃, R₄, and X₂ in the compound of formula (IL) are as defined hereinabove for the compound of formula (I).

Preferably, R₂ and R₃ together with the carbon atom to which they are attached form cyclopropyl. Thus, preferably, the compound of formula (Im) of the present invention is a compound of formula (In):

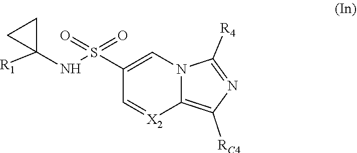

R_{C4}, R₁, R₄, and X₂ in the compound of formula (In) are as defined hereinabove for the compound of formula (I).

Within the scope of the present invention, preferably R₁ is selected from the group consisting of hydrogen, chloro, fluoro, cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)haloalkyl and (C₁₋₂)alkyl, preferably cyano, fluoromethyl and methyl. More preferably, R₁ is cyano.

Thus, preferably within the scope of the present invention the compound of formula (In) is a compound of formula (Io):

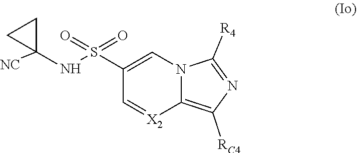

R_{C4}, R₄, and X₂ in the compound of formula (Io) are as defined hereinabove for the compound of formula (I).

Within the scope of the present invention, the compound of formula (IL) or the compound of formula (Im) or the compound of formula (In) wherein R₁ is methyl is also encompassed within the present invention. In certain preferred embodiments of the present invention, R₁ is methyl. Alternatively, within the scope of the present invention, the compound of formula (IL) or the compound of formula (Im) or the compound of formula (In) wherein R₁ is fluoromethyl is also encompassed within the present invention. In certain preferred embodiments of the present invention, R₁ is fluoromethyl.

In one embodiment of the compound of formula (I) of the present invention R₄ is selected from aryl, and heteroaryl. Most preferably, R₄ is heteroaryl. Said aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Preferably, R₄ is a five membered heteroaryl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). The said five membered heteroaryl is preferably selected from imidazolyl, isoxazolyl, pyrazolyl, 1,2,3-triazolyl, 1,2,4-triazolyl, thiazolyl, 1,2,4-oxadiazolyl, 1,3,4-oxadiazolyl, 1,2,4-thiadiazolyl, or 1,3,4-thiadiazolyl. More preferably, said five membered heteroaryl is 1,2,4-thiadiazolyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably optionally substituted with C₁₋₅ alkyl, C₁₋₅ haloalkyl, —O(C₁₋₅ alkyl), —SH, —S(C₁₋₅ alkyl), more preferably optionally substituted with C₁₋₅ alkyl, C₁₋₅ haloalkyl, even more preferably optionally substituted with C₁₋₅ haloalkyl, preferably selected from —CH₂F, —CHF₂ and CF₃, most preferably optionally substituted with —CHF₂.

Thus, in a preferred embodiment, the compound of formula (I) is a compound of formula (Ip):

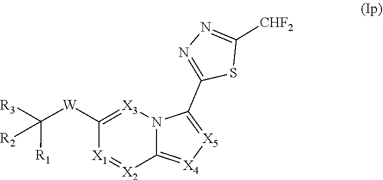

W, R₁, R₂, R₃, X₁, X₂, X₃, X₄, and X₅ in the compound of formula (Ip) are as defined hereinabove for the compound of formula (I).

Preferably W is —NHS(O)₂—. Thus, within the scope of the present invention, the compound of formula (Ip) is a compound of formula (Iq):

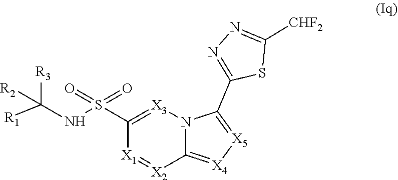

R₁, R₂, R₃, X₁, X₂, X₃, X₄, and X₅ in the compound of formula (Io) are as defined hereinabove for the compound of formula (I).

In a further preferred embodiment, R₂ and R₃ together with the carbon atom to which they are attached form cyclopropyl. Thus, in a preferred embodiment, the compound of formula (Iq) of the present invention is a compound according to formula (Ir):

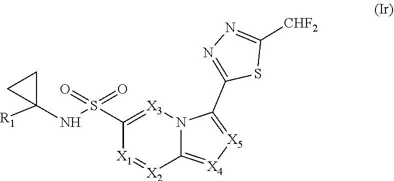

R₁, X₁, X₂, X₃, X₄, and X₅ in the compound of formula (Ir) are as defined hereinabove for the compound of formula (I).

Further preferably, R₁ is selected from the group consisting of hydrogen, chloro, fluoro, cyano, formyl, (C₁₋₂)alkyl, (C₂)alkenyl, (C₂)alkynyl and (C₁₋₂)haloalkyl. Preferably, R₁ is selected from the group consisting of cyano, (C₁₋₂)haloalkyl and (C₁₋₂)alkyl, preferably cyano, fluoromethyl and methyl. More preferably, R₁ is cyano.

Thus, preferably within the scope of the present invention the compound of formula (Ir) is a compound of formula (Is):

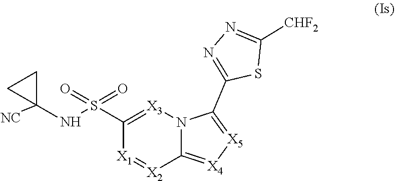

X₁, X₂, X₃, X₄, and X₅ in the compound of formula (Ir) are as defined hereinabove for the compound of formula (I).

It is noted that within the scope of the present invention, the compound of formula (Ip) or the compound of formula (Iq) or the compound of formula (Ir) wherein R₁ is methyl is also encompassed. In certain preferred embodiments of the present invention, R₁ is methyl. It is further noted that alternatively within the scope of the present invention, the compound of formula (Ip) or the compound of formula (Iq) or the compound of formula (Ir) wherein R₁ is fluoromethyl is also encompassed. In certain preferred embodiments of the present invention, R₁ is fluoromethyl.

Further preferred within the scope of the present invention are embodiments wherein X₁ and X₃ are each CH.

Thus, the compound of formula (Ip) of the present invention further relates to an embodiment wherein the compound of formula (Ip) is a compound of formula (It):

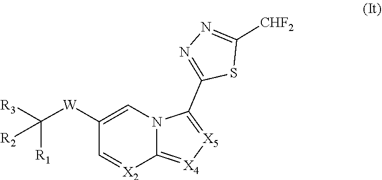

W, R₁, R₂, R₃, X₂, X₄, and X₅ in the compound of formula (It) are as defined hereinabove for the compound of formula (I).

Preferably, W is —NHS(O)₂—. Thus further within the scope of the present invention, the compound of formula (Iq) of the present invention further relates to an embodiment wherein the compound of formula (Iq) is or a compound of formula (Iu),

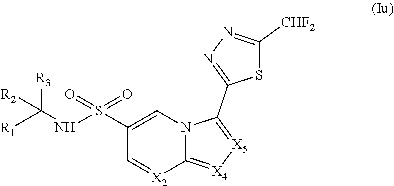

R₁, R₂, R₃, X₂, X₄, and X₅ in the compound of formula (Iu) are as defined hereinabove for the compound of formula (I).

Preferably in the compound of formula (Iu) R₂ and R₃ together with the carbon atom to which they are attached form cyclopropyl.

As thus further encompassed by the present invention, the compound of formula (Ir) of the present invention further relates to an embodiment wherein the compound of formula (Ir) is a compound of formula (Iv),

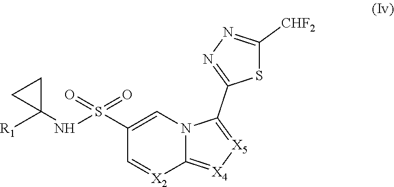

R₁, X₂, X₄, and X₅ in the compound of formula (Iv) are as defined hereinabove for the compound of formula (I).

As preferably R₁ is cyano, the compound of formula (Is) of the present invention further relates to an embodiment wherein the compound of formula (Is) is or a compound of formula (Iw)

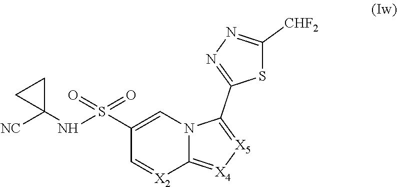

X₂, X₄, and X₅ in the compound of formula (Iw) are as defined hereinabove for the compound of formula (I).

Within the scope of the present invention, the compound of formula (It) or the compound of formula (Iu) or the compound of formula (Iv) wherein R₁ is methyl is also encompassed. In certain preferred embodiments of the present invention, R₁ is methyl. Alternatively, within the scope of the present invention, the compound of formula (It) or the compound of formula (Iu) or the compound of formula (Iv) wherein R₁ is fluoromethyl is also encompassed. In certain preferred embodiments of the present invention, R₁ is fluoromethyl.

As encompassed by the present invention, X₄ is N or C—R_{C4} and X₅ is N or C—R_{C5}. Furthermore, as defined hereinabove, preferably not more than one of X₄ and X₅ is N. In certain embodiments of the present invention, X₅ is N. Thus, if X₅ is N, preferably X₄ is C—R_{C4}. Thus, in certain preferred embodiments the compound of formula (It) is a compound of formula (Ix):

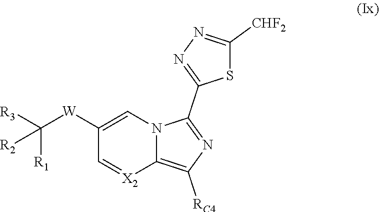

W, R_{C4}, R₁, R₂, R₃, and X₂ in the compound of formula (Ix) are as defined hereinabove for the compound of formula (I).

Further accordingly, as preferably within the scope of the present invention W is —NHS(O)₂—, in certain preferred embodiments the compound of formula (Iu) is a compound of formula (Iy):

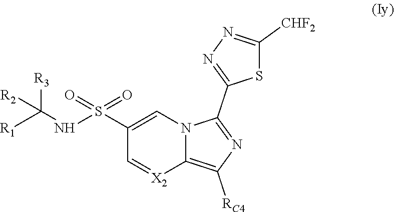

R_{C4}, R₁, R₂, R₃, and X₂ in the compound of formula (Iy) are as defined hereinabove for the compound of formula (I).

Preferably R₂ and R₃ together with the carbon atom to which they are attached form cyclopropyl.

Thus further accordingly, in certain preferred embodiments the compound of formula (Iv) is a compound of formula (Iz):

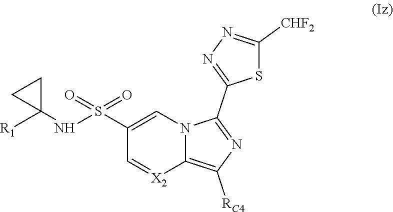

R_{C4}, R₁, and X₂ in the compound of formula (Iz) are as defined hereinabove for the compound of formula (I).

Within the scope of the present invention, preferably R₁ is selected from the group consisting of hydrogen, chloro, fluoro, cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)haloalkyl and (C₁₋₂)alkyl, preferably cyano, fluoromethyl and methyl. More preferably, R₁ is cyano. Thus further accordingly, in certain preferred embodiments the compound of formula (Iw) is a compound of formula (Iaa):

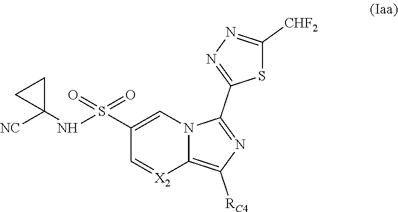

R_{C4}, and X₂ in the compound of formula (Iaa) are as defined hereinabove for the compound of formula (I).

It is however noted that within the scope of the present invention, the compound of formula (Ix) or the compound of formula (Iy) or the compound of formula (Iz) wherein R₁ is methyl is also encompassed. In certain preferred embodiments of the present invention, R₁ is methyl. It is further noted that alternatively within the scope of the present invention, the compound of formula (Ix) or the compound of formula (Iy) or the compound of formula (Iz) wherein R₁ is fluoromethyl is also encompassed. In certain preferred embodiments of the present invention, R₁ is fluoromethyl.

Preferably within the scope of the present invention X₂ is C—Y_{C2}—R_{C2}. Most preferably, —Y_{C2}—R_{C2} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.

Thus, preferably the compound of formula (I) of the present invention is a compound of formula (Iab):

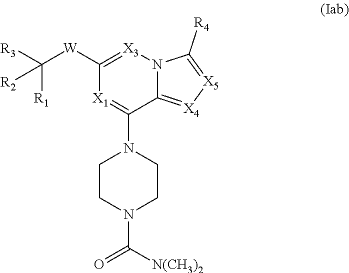

W, R₁, R₂, R₃, R₄, X₁, X₃, X₄, and X₅ in the compound of formula (Iab) are as defined for the compound of formula (I) of the present invention.

Preferably W is —NHS(O)₂—. Accordingly, preferably the compound of formula (Ia) of the present invention or the compound of formula (Iab) is a compound of formula (Iac):

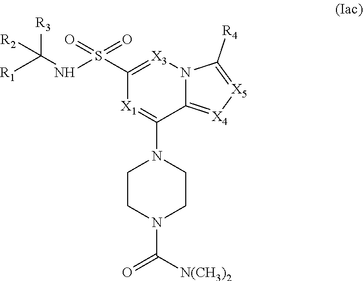

R₁, R₂, R₃, R₄, X₁, X₃, X₄, and X₅ in the compound of formula (Iac) are as defined for the compound of formula (I) of the present invention.

Preferably, R₂ and R₃ together with the carbon atom to which they are attached form cyclopropyl. Thus further accordingly, preferably the compound of formula (Iac) or the compound of formula (Ib) of the present invention is a compound of formula (Iad):

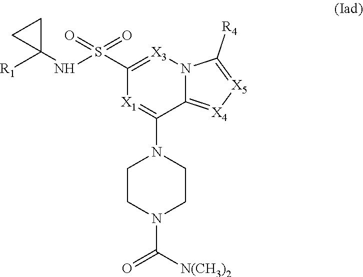

R₁, R₄, X₁, X₃, X₄, and X₅ in the compound of formula (Iad) are as defined for the compound of formula (I) of the present invention.

Further accordingly, preferably, R₁ is cyano, thus preferably the compound of formula (Ic) of the present invention or the compound of formula (Iad) of the present invention is a compound of formula (Iae):

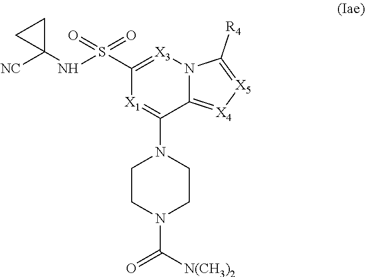

R₄, X₁, X₃, X₄, and X₅ in the compound of formula (Iae) are as defined for the compound of formula (I) of the present invention.

It is however noted that within the scope of the present invention, the compound of formula (Iab) or the compound of formula (Iac) or the compound of formula (Iad) wherein R₁ is methyl is also encompassed. In certain preferred embodiments of the present invention, R₁ is methyl. Alternatively, it is noted that within the scope of the present invention, the compound of formula (Iab) or the compound of formula (Iac) or the compound of formula (Iad) wherein R₁ is fluoromethyl is also encompassed. In certain preferred embodiments of the present invention, R₁ is fluoromethyl.

Further preferred within the scope of the present invention are embodiments wherein X₁ and X₃ are each CH.

Thus, accordingly the compound of formula (Iac) of the present invention is preferably a compound of formula (Iag):

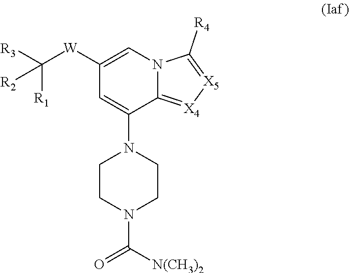

W, R₁, R₂, R₃, R₄, X₄, and X₅ in the compound of formula (Iaf) are as defined for the compound of formula (I) of the present invention.

Preferably W is —NHS(O)₂—. Further accordingly, the compound of formula (Iad) of the present invention or the compound of formula (Iaf) as defined herein is preferably a compound of formula (Iag):

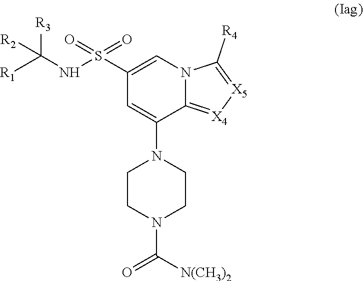

R₁, R₂, R₃, R₄, X₄, and X₅ in the compound of formula (Iag) are as defined for the compound of formula (I) of the present invention.

Preferably, R₂ and R₃ together with the carbon atom to which they are attached form cyclopropyl.

Further accordingly, the compound of formula (Iae) or the compound of formula (Iag) of the present invention is preferably a compound of formula (Iah):

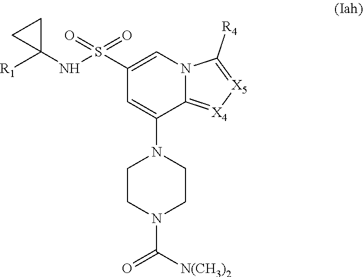

R₁, R₄, X₄, and X₅ in the compound of formula (Iah) are as defined for the compound of formula (I) of the present invention.

Preferably, R₁ is cyano. Thus, further accordingly, the compound of formula (Iaf) of the present invention or the compound of formula (Iah) of the present invention is preferably a compound of formula (Iai):

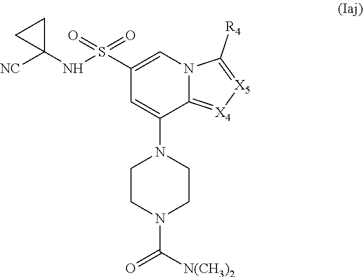

R₄, X₄, and X₅ in the compound of formula (Iai) are as defined for the compound of formula (I) of the present invention.

Within the scope of the present invention, the compound of formula (Iaf) or the compound of formula (Iag) or the compound of formula (Iah) wherein R₁ is methyl is also encompassed. In certain preferred embodiments of the present invention, R₁ is methyl. Alternatively, within the scope of the present invention, the compound of formula (Iaf) or the compound of formula (Iag) or the compound of formula (Iah) wherein R₁ is fluoromethyl is also encompassed. In certain preferred embodiments of the present invention, R₁ is fluoromethyl.

As further encompassed by the present invention, X₄ is N or C—R_{C4} and X₅ is N or C—R_{C5}. Furthermore, as defined hereinabove, preferably not more than one of X₄ and X₅ is N. In certain embodiments of the present invention, X₅ is N. Thus, if X₅ is N, preferably X₄ is C—R_{C4}.

Thus accordingly, the compound of formula (Iab) of the present invention is preferably a compound of formula (Iaj):

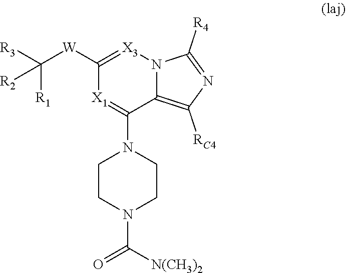

W, R_{C4}, R₁, R₂, R₃, R₄, X₁, and X₃ in the compound of formula (Iaj) are as defined for the compound of formula (I) of the present invention.

W is preferably —NHS(O)₂—. Thus further accordingly, the compound of formula (Iac) or the compound of formula (Iaj) of the present invention is preferably a compound of formula (Iak):

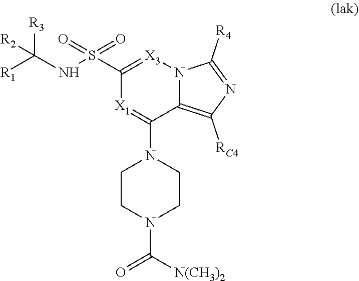

R_{C4}, R₁, R₂, R₃, R₄, X₁, and X₃ in the compound of formula (Iak) are as defined for the compound of formula (I) of the present invention.

As disclosed herein, preferably R₂ and R₃ together with the carbon atom to which they are attached form cyclopropyl. Thus further accordingly, the compound of formula (Iad) or the compound of formula (Iak) of the present invention is preferably a compound of formula (IaL):

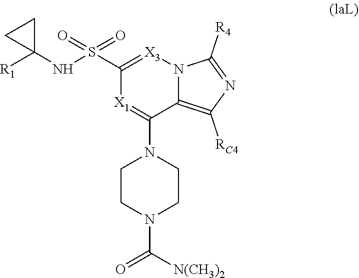

R_{C4}, R₁, R₄, X₁, and X₃ in the compound of formula (IaL) are as defined for the compound of formula (I) of the present invention.

Further preferably R₁ is cyano. Thus further accordingly, the compound of formula (Iae) or the compound of formula (IaL) of the present invention is preferably a compound of formula (Iam):

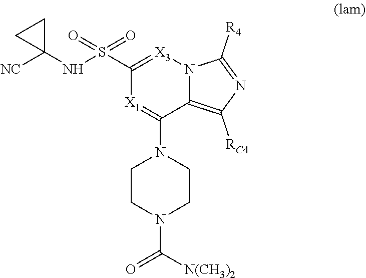

R_{C4}, R₄, X₁, and X₃ in the compound of formula (Iam) are as defined for the compound of formula (I) of the present invention.

Within the scope of the present invention, the compound of formula (Iaj) or the compound of formula (Iak) or the compound of formula (IaL) wherein R₁ is methyl is also encompassed. In certain preferred embodiments of the present invention, R₁ is methyl. Further within the scope of the present invention, the compound of formula (Iaj) or the compound of formula (Iak) or the compound of formula (IaL) wherein R₁ is fluoromethyl is also encompassed. In certain preferred embodiments of the present invention, R₁ is fluoromethyl.

Further accordingly, the compound of formula (Iaf) of the present invention is preferably a compound of formula (Ian):

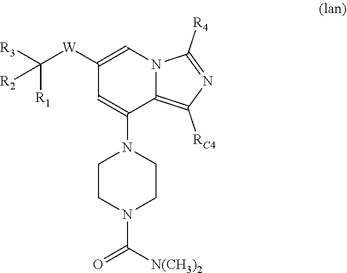

W, R₁, R₂, R₃, R₄, and R_{C4} in the compound of formula Ian are as defined for the compound of formula (I) of the present invention.

Further accordingly, the compound of formula (Iag) of the present invention is preferably a compound of formula (Iao):

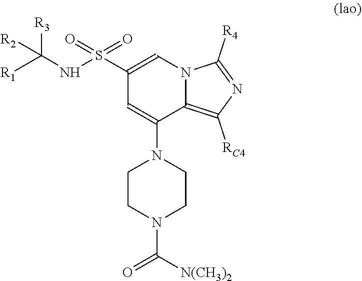

R₁, R₂, R₃, R₄, and R_{C4} in the compound of formula (Iao) are as defined for the compound of formula (I) of the present invention.

Further accordingly, the compound of formula (Iah) of the present invention is preferably a compound of formula (Iap):

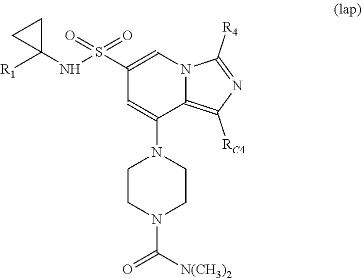

R₁, R₄, and R_{C4} in the compound of formula (Iap) are as defined for the compound of formula (I) of the present invention.

Further accordingly, the compound of formula (Iai) of the present invention is preferably a compound of formula (Iaq):

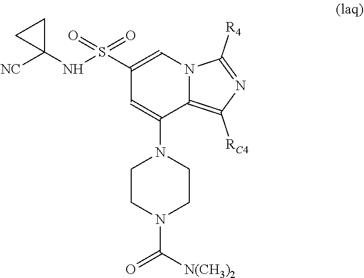

R₄, and R_{C4} in the compound of formula (Iaq) are as defined for the compound of formula (I) of the present invention.

As further encompassed by the present invention, X₄ is N or C—R_{C4} and X₅ is N or C—R_{C5}.

Furthermore, as defined hereinabove, preferably not more than one of X₄ and X₅ is N. In certain embodiments of the present invention, X₄ is N. Thus, if X₄ is N, preferably X₅ is C—R_{C5}.

Thus accordingly, in certain preferred embodiments of the present invention, the compound of formula (I) of the present invention is a compound of formula (Iar):

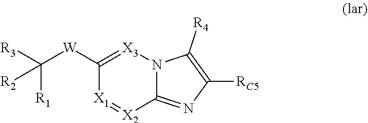

W, R₁, R₂, R₃, R₄, X₁, X₂, X₃, and R_{C5} in the compound of formula (Iar) are as defined for the compound of formula (I) of the present invention.

Further accordingly, as W is preferably —NHS(O)₂—, in certain preferred embodiments of the present invention, the compound of formula (Ia) of the present invention is a compound of formula (Ias):

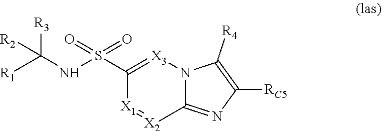

R₁, R₂, R₃, R₄, X₁, X₂, X₃, and R_{C5} in the compound of formula (Iar) are as defined for the compound of formula (I) of the present invention.

Further accordingly, as preferably R₂ and R₃ together with the carbon atom to which they are attached form cyclopropyl, in certain preferred embodiments of the present invention, the compound of formula (Ib) of the present invention is a compound of formula (Iat):

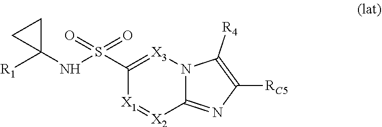

R₁, R₄, X₁, X₂, X₃, and R_{C5} in the compound of formula (Iat) are as defined for the compound of formula (I) of the present invention.

Further accordingly, as preferably R₁ is cyano, in certain preferred embodiments of the present invention, the compound of formula (Ic) of the present invention is a compound of formula (Iau):

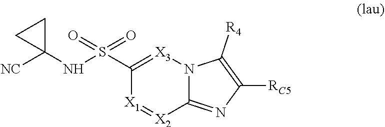

R₄, X₁, X₂, X₃, and R_{C5} in the compound of formula (Iau) are as defined for the compound of formula (I) of the present invention.

However, within the scope of the present invention, the compound of formula (Iar) or the compound of formula (Ias) or the compound of formula (Iat) wherein R₁ is methyl is also encompassed.

In certain preferred embodiments of the present invention, R₁ is methyl. Further within the scope of the present invention, the compound of formula (Iar) or the compound of formula (Ias) or the compound of formula (Iat) wherein R₁ is fluoromethyl is also encompassed. In certain preferred embodiments of the present invention, R₁ is fluoromethyl.

Further accordingly, in certain preferred embodiments of the present invention, the compound of formula (Id) of the present invention is a compound of formula (Iav):

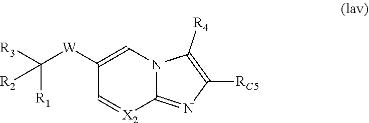

W, R₁, R₂, R₃, R₄, X₂, and R_{C5} in the compound of formula (Iav) are as defined for the compound of formula (I) of the present invention.

Further accordingly, in certain preferred embodiments of the present invention, the compound of formula (Ie) of the present invention is a compound of formula (Iaw):

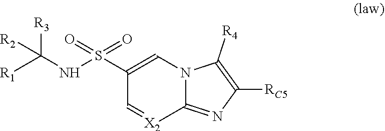

R₁, R₂, R₃, R₄, X₂, and R_{C5} in the compound of formula (Iaw) are as defined for the compound of formula (I) of the present invention.

Further accordingly, in certain preferred embodiments of the present invention, the compound of formula (If) of the present invention is a compound of formula (Iax):

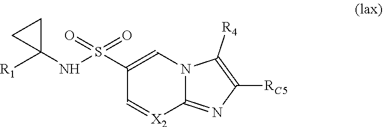

R₁, R₄, X₂, and R_{C5} in the compound of formula (Iax) are as defined for the compound of formula (I) of the present invention.

Further accordingly, in certain preferred embodiments of the present invention, the compound of formula (Ig) of the present invention is a compound of formula (Iay):

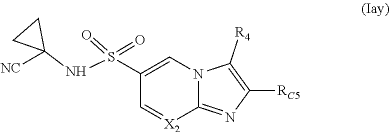

R₄, X₂, and R_{C5} in the compound of formula (Iay) are as defined for the compound of formula (I) of the present invention.

Further accordingly, in certain preferred embodiments of the present invention, the compound of formula (Ip) of the present invention is a compound of formula (Iaz):

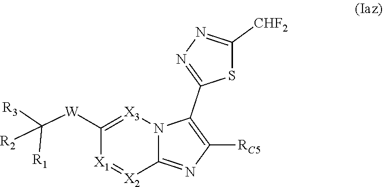

W, R₁, R₂, R₃, X₁, X₂, X₃, and R_{C5} in the compound of formula (Iaz) are as defined for the compound of formula (I) of the present invention.

Further accordingly, as preferably W is —NHS(O)₂—, in certain preferred embodiments of the present invention, the compound of formula (Iq) of the present invention is a compound of formula (Iba):

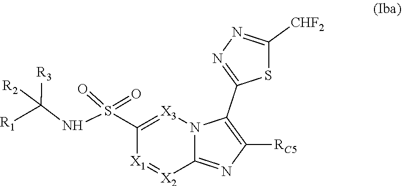

R₁, R₂, R₃, X₁, X₂, X₃, and R_{C5} in the compound of formula (Iba) are as defined for the compound of formula (I) of the present invention.

Further accordingly, as preferably R₂ and R₃ together with the carbon atom to which they are attached form cyclopropyl, in certain preferred embodiments of the present invention, the compound of formula (Ir) of the present invention is a compound of formula (Ibb):

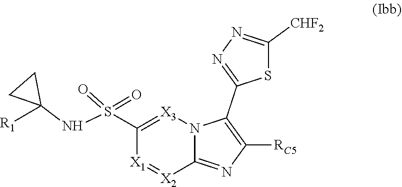

R₁, X₂, and R_{C5} in the compound of formula (Ibb) are as defined for the compound of formula (I) of the present invention.

Further accordingly, as preferably R₁ is cyano, in certain preferred embodiments of the present invention, the compound of formula (Is) of the present invention is a compound of formula (Ibc):

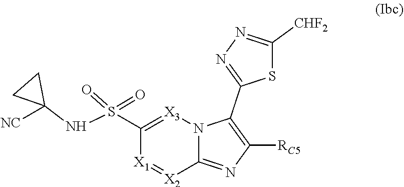

Further preferred within the scope of the present invention are embodiments wherein X₁ and X₃ are each CH. Thus, in certain preferred embodiments, the compound of formula (It) is a compound of formula (Ibd):

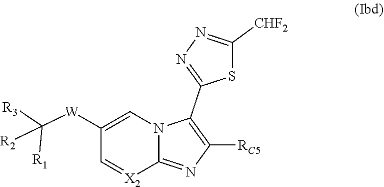

W, R₁, R₂, R₃, X₂, and R_{C5} in the compound of formula (Ibd) are as defined for the compound of formula (I) of the present invention.

Further accordingly, in certain preferred embodiments of the present invention, the compound of formula (Iu) of the present invention is a compound of formula (Ibe):

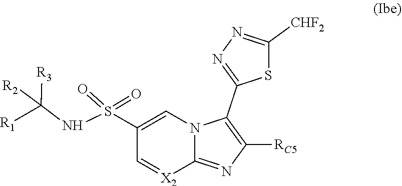

R₁, R₂, R₃, X₂, and R_{C5} in the compound of formula (Ibe) are as defined for the compound of formula (I) of the present invention.

Further accordingly, in certain preferred embodiments of the present invention, the compound of formula (Iv) of the present invention is a compound of formula (Ibf):

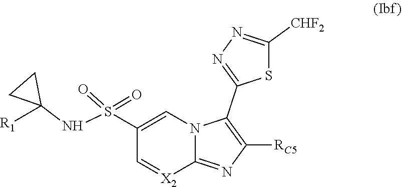

R₁, X₂, and R_{C5} in the compound of formula (Ibf) are as defined for the compound of formula (I) of the present invention.

Further accordingly, in certain preferred embodiments of the present invention, the compound of formula (Iw) of the present invention is a compound of formula (Ibg):

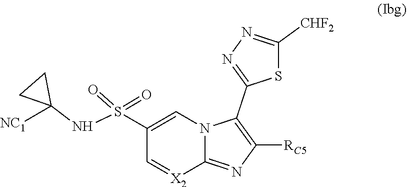

X₂ and R_{C5} in the compound of formula (Ibf) are as defined for the compound of formula (I) of the present invention.

In certain embodiments of the present invention, preferably X₂ is C—Y_{C2}—R_{C2}. Most preferably, —Y_{C2}—R_{C2} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.

Thus accordingly, in certain preferred embodiments of the present invention the compound of formula (Iab) is a compound of formula (Ibh):

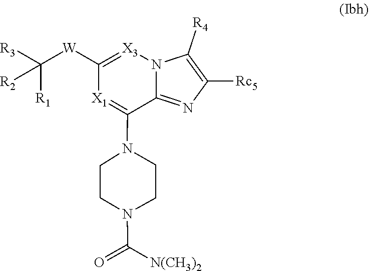

W, R₁, R₂, R₃, R₄, X₁, X₃ and R_{C5} in the compound of formula (Ibh) are as defined for the compound of formula (I) of the present invention.

Further accordingly, in certain preferred embodiments of the present invention the compound of formula (Iac) is a compound of formula (Ibi):

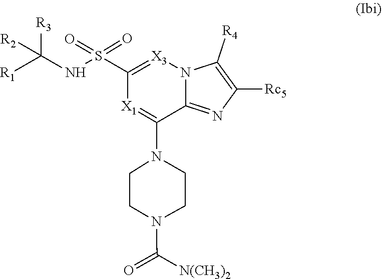

R₁, R₂, R₃, R₄, X₁, X₃ and R_{C5} in the compound of formula (Ibi) are as defined for the compound of formula (I) of the present invention.

Further accordingly, in certain preferred embodiments of the present invention the compound of formula (Iad) is a compound of formula (Ibj):

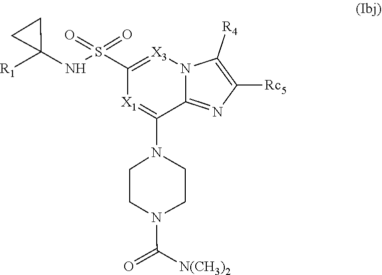

R₁, R₄, X₁, X₃ and R_{C5} in the compound of formula (Ibi) are as defined for the compound of formula (I) of the present invention.

Further accordingly, in certain preferred embodiments of the present invention the compound of formula (Iae) is a compound of formula (Ibk):

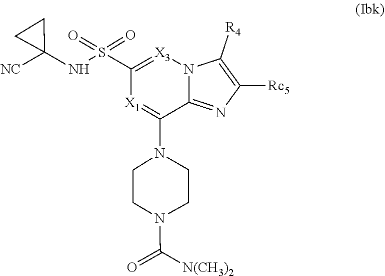

R₄, X₁, X₃ and R_{C5} in the compound of formula (Ibi) are as defined for the compound of formula (I) of the present invention.

Further preferred within the scope of the present invention are embodiments wherein X₁ and X₃ are each CH. Thus, in certain preferred embodiments, the compound of formula (Iaf) is a compound of formula (IbL):

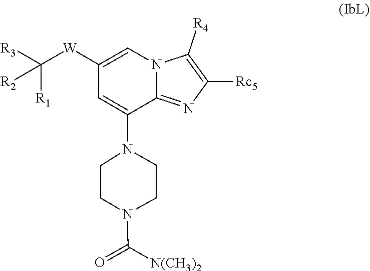

W, R₁, R₂, R₃, R₄, and R_{C5} in the compound of formula (IbL) are as defined for the compound of formula (I) of the present invention.

Further accordingly, in certain preferred embodiments of the present invention the compound of formula (Iag) is a compound of formula (Ibm):

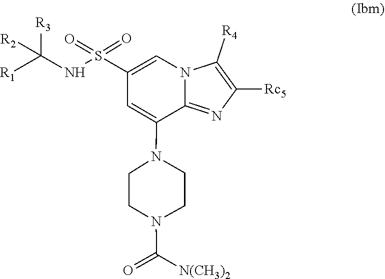

R₁, R₂, R₃, R₄, and R_{C5} in the compound of formula (Ibm) are as defined for the compound of formula (I) of the present invention.

Further accordingly, in certain preferred embodiments of the present invention the compound of formula (Iah) is a compound of formula (Ibn):

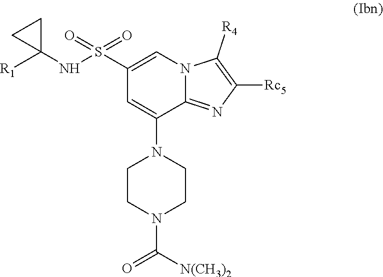

R₁, R₄, and R_{C5} in the compound of formula (Ibn) are as defined for the compound of formula (I) of the present invention.

Further accordingly, in certain preferred embodiments of the present invention the compound of formula (Iai) is a compound of formula (Ibo):

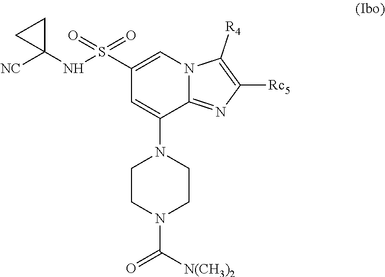

R₄, and R_{C5} in the compound of formula (Ibn) are as defined for the compound of formula (I) of the present invention.

In certain embodiments of the present invention, X₁ is CF and X₃ is CH. Thus, the compound of formula (I) is a compound of formula (Ibp):

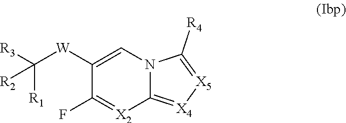

W, R₁, R₂, R₃, R₄, X₂, X₄, and X₅ in the compound of formula (Ibp) are as defined hereinabove for the compound of formula (I).

Further accordingly, as preferably W is —NHS(O)₂—, in certain preferred embodiments of the present invention, the compound of formula (Ibp) of the present invention is a compound of formula (Ibq):

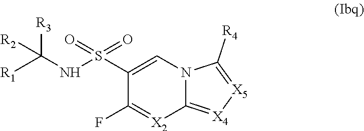

R₁, R₂, R₃, R₄, X₂, X₄, and X₅ in the compound of formula (Ibq) are as defined hereinabove for the compound of formula (I).

Further accordingly, as preferably R₂ and R₃ together with the carbon atom to which they are attached form cyclopropyl, in certain preferred embodiments of the present invention, the compound of formula (Ibq) of the present invention is a compound of formula (Ibr):

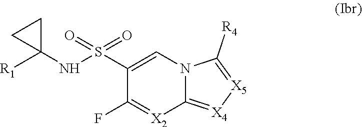

R₁, R₄, X₂, X₄, and X₅ in the compound of formula (Ibr) are as defined hereinabove for the compound of formula (I).

Further accordingly, as preferably R₁ is cyano, in certain preferred embodiments of the present invention, the compound of formula (Ibr) of the present invention is a compound of formula (Ibs):

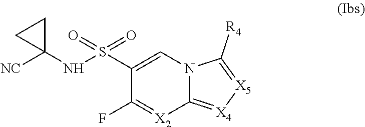

R₄, X₂, X₄, and X₅ in the compound of formula (Ibs) are as defined hereinabove for the compound of formula (I).

It is however noted that the compounds of formula (Ibp), (Ibq) or (Ibr) wherein R₁ is methyl or fluoromethyl are also encompassed within the scope of the present invention.

In certain embodiments of the present invention, preferably X₂ is C—Y_{C2}—R_{C2}. Most preferably, —Y_{C2}—R_{C2} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.

Thus accordingly, in certain embodiments of the present invention the compound of formula (Ibp) is a compound of formula (Ibt):

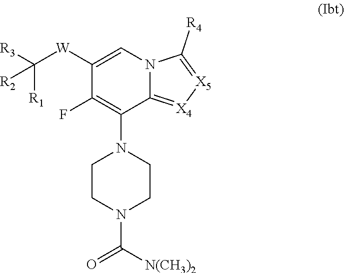

W, R₁, R₂, R₃, R₄, X₄, and X₅ in the compound of formula (Ibt) are as defined hereinabove for the compound of formula (I).

Further accordingly, in certain embodiments of the present invention the compound of formula (Ibq) is a compound of formula (Ibu):

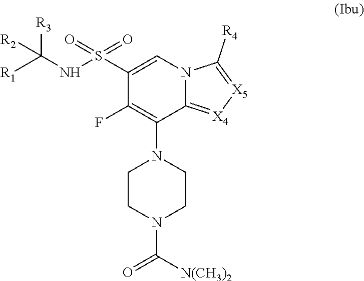

R₁, R₂, R₃, R₄, X₄, and X₅ in the compound of formula (Ibu) are as defined hereinabove for the compound of formula (I).

Further accordingly, in certain embodiments of the present invention the compound of formula (Ibr) is a compound of formula (Ibv).

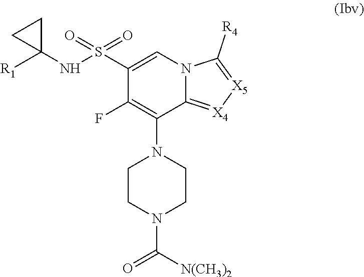

R₁, R₄, X₄, and X₅ in the compound of formula (Ibv) are as defined hereinabove for the compound of formula (I).

Further accordingly, in certain embodiments of the present invention the compound of formula (Ibs) is a compound of formula (Ibw).

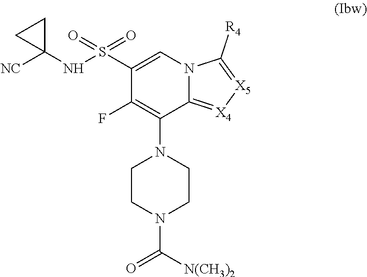

R₄, X₄, and X₅ in the compound of formula (Ibt) are as defined hereinabove for the compound of formula (I).

It is however noted that the compounds of formula (Ibt), (Ibu) or (Ibv) wherein R₁ is methyl or fluoromethyl are also encompassed within the scope of the present invention.

In certain preferred embodiments, X₄ is C—R_{C4} and X₅ is C—R_{C5}. Thus, the compound of formula (I) is in certain embodiments a compound of formula (Ibx):

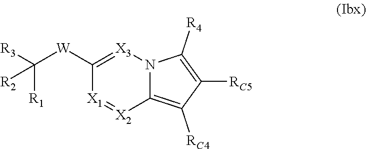

W, R₁, R₂, R₃, R₄, R_{C4}, R_{C5}, X₁, X₂, and X₃ in the compound of formula (Ibx) are as defined hereinabove for the compound of formula (I).

Further accordingly, as preferably W is —NHS(O)₂—, in certain preferred embodiments of the present invention, the compound of formula (Ibx) of the present invention is a compound of formula (Iby):

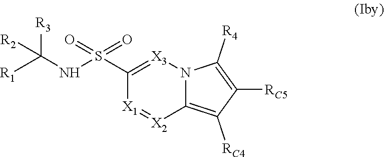

R₁, R₂, R₃, R₄, R_{C4}, R_{C5}, X₁, X₂, and X₃ in the compound of formula (Iby) are as defined hereinabove for the compound of formula (I).

Further accordingly, as preferably R₂ and R₃ together with the carbon atom to which they are attached form cyclopropyl, in certain preferred embodiments of the present invention, the compound of formula (Iby) of the present invention is a compound of formula (Ibz):

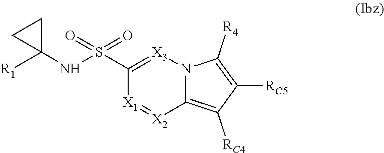

R₁, R₄, R_{C4}, R_{C5}, X₁, X₂, and X₃ in the compound of formula (Ibz) are as defined hereinabove for the compound of formula (I).

Further accordingly, as preferably R₁ is cyano, in certain preferred embodiments of the present invention, the compound of formula (Ibz) of the present invention is a compound of formula (Ica):

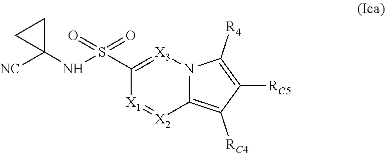

R₄, R_{C4}, RCM, X₁, X₂, and X₃ in the compound of formula (Ica) are as defined hereinabove for the compound of formula (I).

It is however noted that the compounds of formula (Ibx), (Iby) or (Ibz) wherein R₁ is methyl or fluoromethyl are also encompassed within the scope of the present invention.

In certain preferred embodiments X₁ is CH and X₃ is CH.

Thus accordingly, in certain embodiments of the present invention the compound of formula (Ibx) is a compound of formula (Icb):

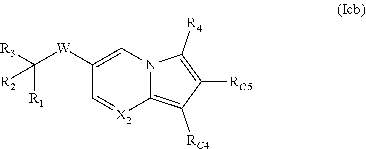

W, R₁, R₂, R₃, R₄, R_{C4}, R_{C5}, and X₂ in the compound of formula (Icb) are as defined hereinabove for the compound of formula (I).

Further accordingly, in certain embodiments of the present invention the compound of formula (Iby) is a compound of formula (Icc):

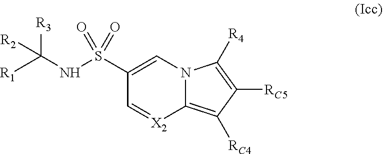

R₁, R₂, R₃, R₄, R_{C4}, R_{C5}, and X₂ in the compound of formula (Icc) are as defined hereinabove for the compound of formula (I).

Further accordingly, in certain embodiments of the present invention the compound of formula (Ibz) is a compound of formula (Icd):

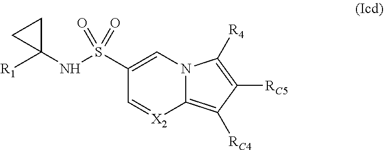

R₁, R₄, R_{C4}, R_{C5}, and X₂ in the compound of formula (Icd) are as defined hereinabove for the compound of formula (I).

Further accordingly, in certain embodiments of the present invention the compound of formula (Ica) is a compound of formula (Ice):

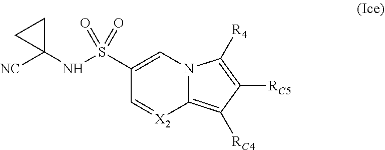

R₄, R_{C4}, R_{C5}, and X₂ in the compound of formula (Ice) are as defined hereinabove for the compound of formula (I).

In certain preferred embodiments X₄ is CH and X₅ is CH.

Thus accordingly, in certain embodiments of the present invention the compound of formula (Icb) is a compound of formula (Icf):

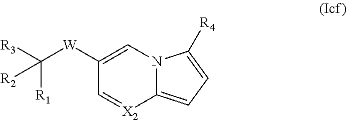

W, R₁, R₂, R₃, R₄, and X₂ in the compound of formula (Icf) are as defined hereinabove for the compound of formula (I).

Further accordingly, in certain embodiments of the present invention the compound of formula (Icc) is a compound of formula (Icg):

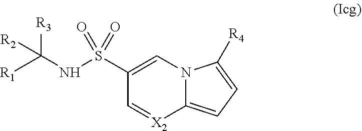

R₁, R₂, R₃, R₄, and X₂ in the compound of formula (Icg) are as defined hereinabove for the compound of formula (I).

Further accordingly, in certain embodiments of the present invention the compound of formula (Icd) is a compound of formula (Ich):

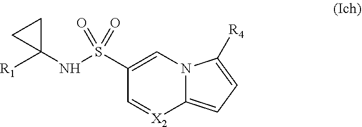

R₁, R₄, and X₂ in the compound of formula (Ich) are as defined hereinabove for the compound of formula (I).

Further accordingly, in certain embodiments of the present invention the compound of formula (Icd) is a compound of formula (Ici):

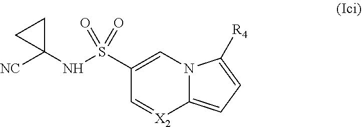

R₄ and X₂ in the compound of formula (Ici) are as defined hereinabove for the compound of formula (I).

Preferred compound of formula (I) are selected from the following compounds:

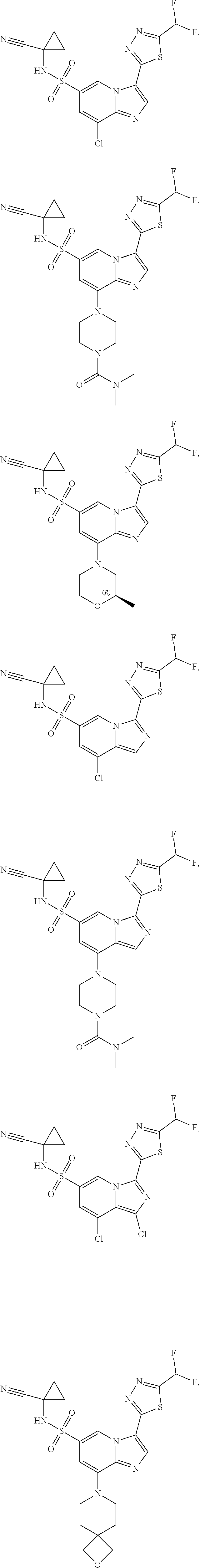

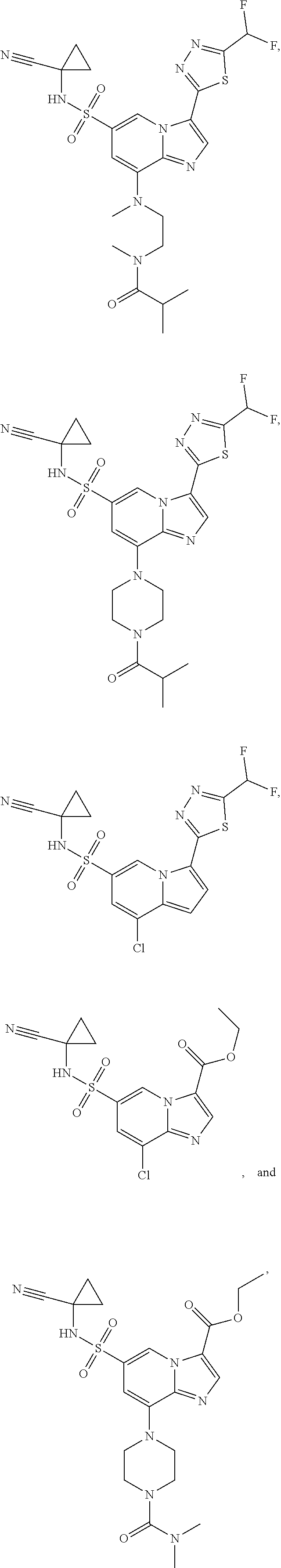

or an enantiomer, diastereoisomer, tautomer, pharmaceutically acceptable solvate, pharmaceutically acceptable crystal form, pharmaceutically acceptable salt or a prodrug thereof.

Further preferred compounds of formula (I) are selected from:

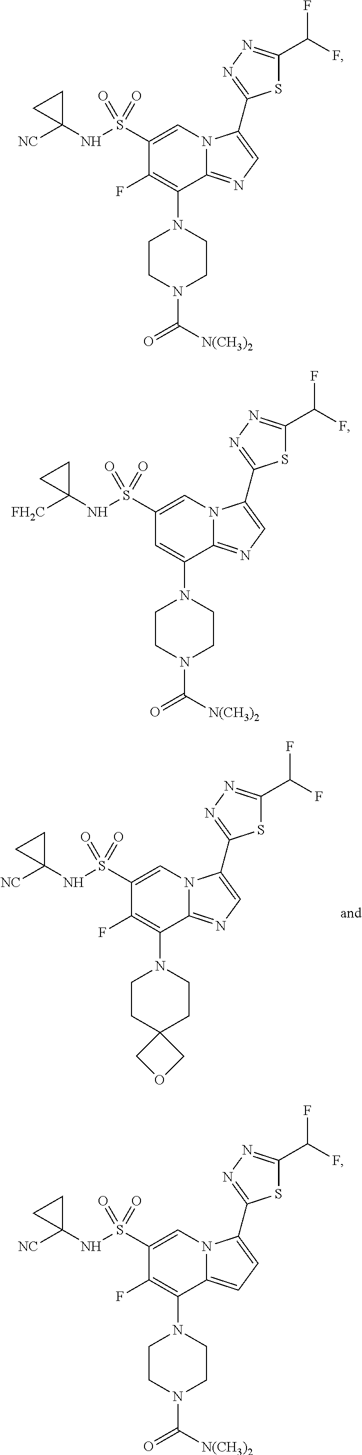

or an enantiomer, diastereoisomer, tautomer, pharmaceutically acceptable solvate, pharmaceutically acceptable crystal form, pharmaceutically acceptable salt or a prodrug thereof.

Further preferred compounds of formula (I) are selected from:

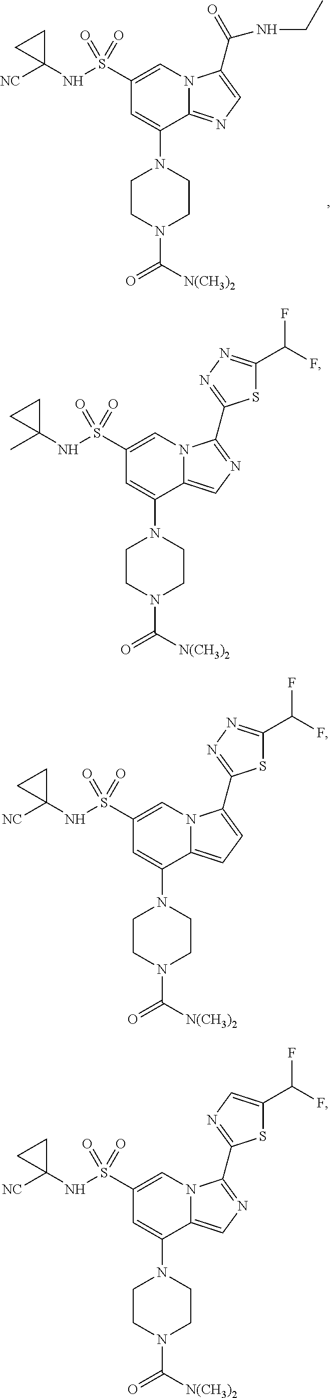

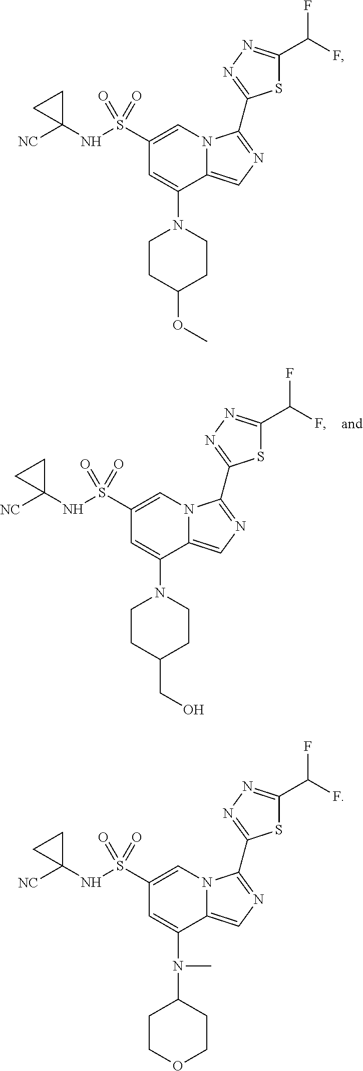 [SMILES: COC1CCN(c2cc(S(=O)(=O)NC3(C#N)CC3)cn3c(-c4nnc(C(F)F)s4)ncc23)CC1 ; N#CC1(NS(=O)(=O)c2cc(N3CCC(CO)CC3)c3cnc(-c4nnc(C(F)F)s4)n3c2)CC1 ; "and" ; CN(c1cc(S(=O)(=O)NC2(C#N)CC2)cn2c(-c3nnc(C(F)F)s3)ncc12)C1CCOCC1]

Further preferred compounds of formula (I) are selected from:

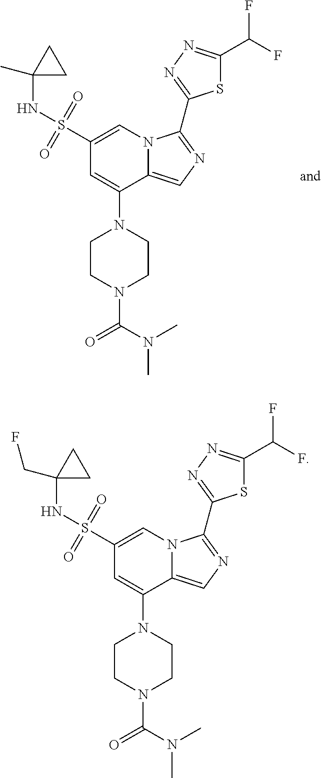 [SMILES: CN(C)C(=O)N1CCN(c2cc(S(=O)(=O)NC3(C)CC3)cn3c(-c4nnc(C(F)F)s4)ncc23)CC1 ; CN(C)C(=O)N1CCN(c2cc(S(=O)(=O)NC3(CF)CC3)cn3c(-c4nnc(C(F)F)s4)ncc23)CC1]

Further preferred compounds of formula (I) are selected from

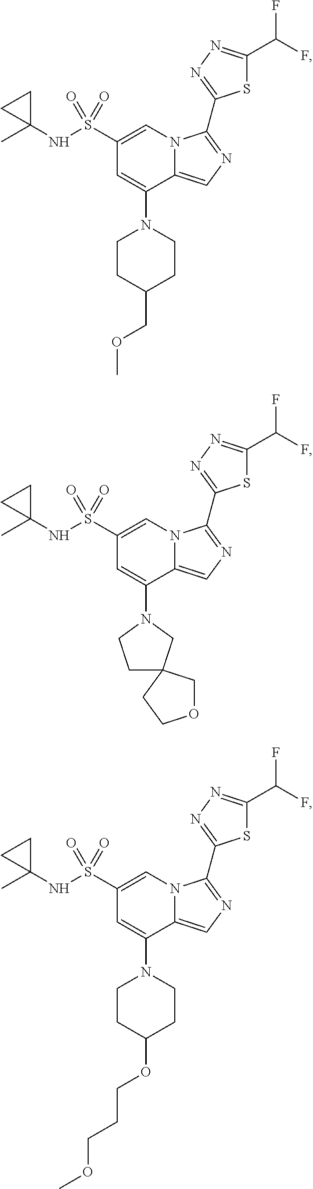 [SMILES: COCC1CCN(c2cc(S(=O)(=O)NC3(C)CC3)cn3c(-c4nnc(C(F)F)s4)ncc23)CC1 ; CC1(NS(=O)(=O)c2cc(N3CCC4(CCOC4)C3)c3cnc(-c4nnc(C(F)F)s4)n3c2)CC1 ; COCCCOC1CCN(c2cc(S(=O)(=O)NC3(C)CC3)cn3c(-c4nnc(C(F)F)s4)ncc23)CC1]

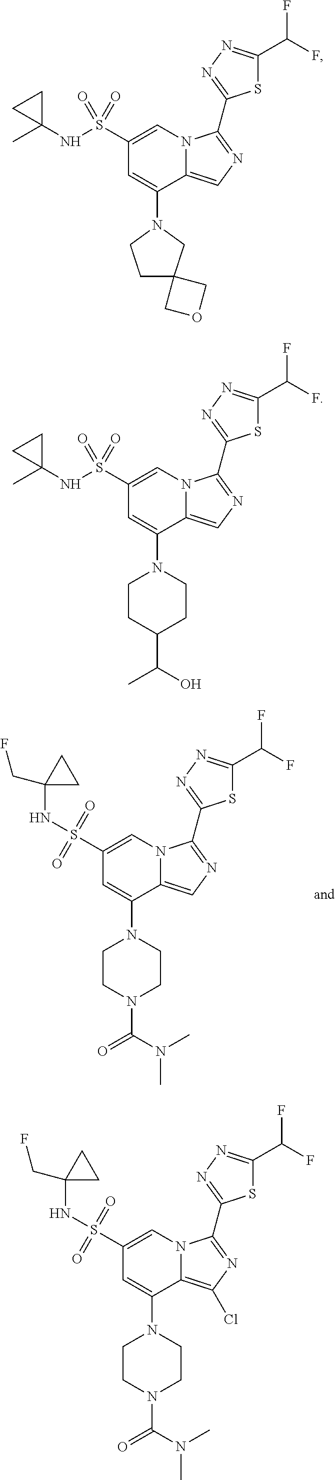 [SMILES: CC1(NS(=O)(=O)c2cc(N3CCC4(COC4)C3)c3cnc(-c4nnc(C(F)F)s4)n3c2)CC1 ; CC(O)C1CCN(c2cc(S(=O)(=O)NC3(C)CC3)cn3c(-c4nnc(C(F)F)s4)ncc23)CC1 ; CN(C)C(=O)N1CCN(c2cc(S(=O)(=O)NC3(CF)CC3)cn3c(-c4nnc(C(F)F)s4)ncc23)CC1 ; CN(C)C(=O)N1CCN(c2cc(S(=O)(=O)NC3(CF)CC3)cn3c(-c4nnc(C(F)F)s4)nc(Cl)c23)CC1]

Further preferred compounds of formula (I) are selected from

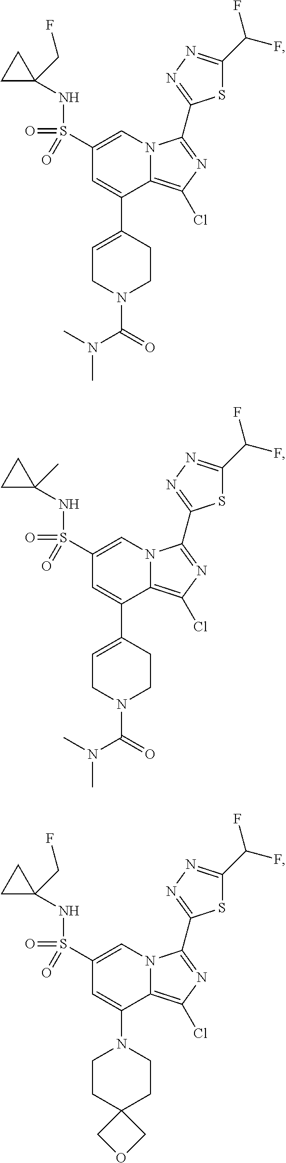 [SMILES: CN(C)C(=O)N1CC=C(c2cc(S(=O)(=O)NC3(CF)CC3)cn3c(-c4nnc(C(F)F)s4)nc(Cl)c23)CC1 ; CN(C)C(=O)N1CC=C(c2cc(S(=O)(=O)NC3(C)CC3)cn3c(-c4nnc(C(F)F)s4)nc(Cl)c23)CC1 ; O=S(=O)(NC1(CF)CC1)c1cc(N2CCC3(CC2)COC3)c2c(Cl)nc(-c3nnc(C(F)F)s3)n2c1]

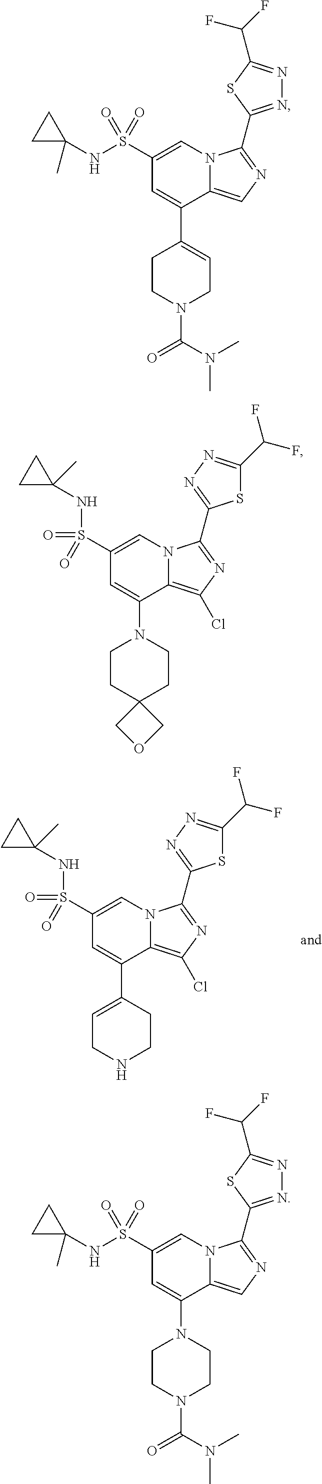 [SMILES: CN(C)C(=O)N1CC=C(c2cc(S(=O)(=O)NC3(C)CC3)cn3c(-c4nnc(C(F)F)s4)ncc23)CC1 ; CC1(NS(=O)(=O)c2cc(N3CCC4(CC3)COC4)c3c(Cl)nc(-c4nnc(C(F)F)s4)n3c2)CC1 ; CC1(NS(=O)(=O)c2cc(C3=CCNCC3)c3c(Cl)nc(-c4nnc(C(F)F)s4)n3c2)CC1 ; CN(C)C(=O)N1CCN(c2cc(S(=O)(=O)NC3(C)CC3)cn3c(-c4nnc(C(F)F)s4)ncc23)CC1]

Further preferred compound of formula (I) is

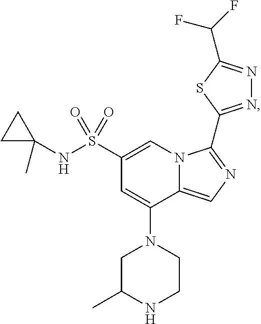 [SMILES: CC1CN(c2cc(S(=O)(=O)NC3(C)CC3)cn3c(-c4nnc(C(F)F)s4)ncc23)CCN1]

preferably

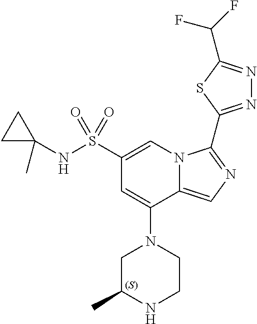

Particularly preferred are exemplified compounds, i.e. the compounds 1 to 299, as described hereinbelow. Preferably, the compound of formula (I) is selected from:

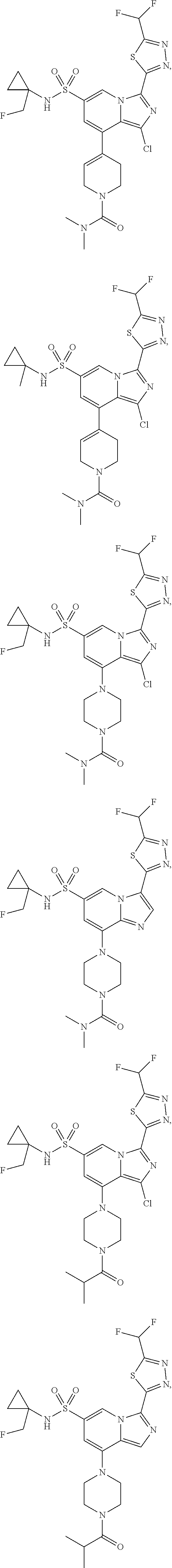 [SMILES: CN(C)C(=O)N1CC=C(c2cc(S(=O)(=O)NC3(CF)CC3)cn3c(-c4nnc(C(F)F)s4)nc(Cl)c23)CC1 ; CN(C)C(=O)N1CC=C(c2cc(S(=O)(=O)NC3(C)CC3)cn3c(-c4nnc(C(F)F)s4)nc(Cl)c23)CC1 ; CN(C)C(=O)N1CCN(c2cc(S(=O)(=O)NC3(CF)CC3)cn3c(-c4nnc(C(F)F)s4)nc(Cl)c23)CC1 ; CN(C)C(=O)N1CCN(c2cc(S(=O)(=O)NC3(CF)CC3)cn3c(-c4nnc(C(F)F)s4)cnc23)CC1 ; CC(C)C(=O)N1CCN(c2cc(S(=O)(=O)NC3(CF)CC3)cn3c(-c4nnc(C(F)F)s4)nc(Cl)c23)CC1 ; CC(C)C(=O)N1CCN(c2cc(S(=O)(=O)NC3(CF)CC3)cn3c(-c4nnc(C(F)F)s4)ncc23)CC1]

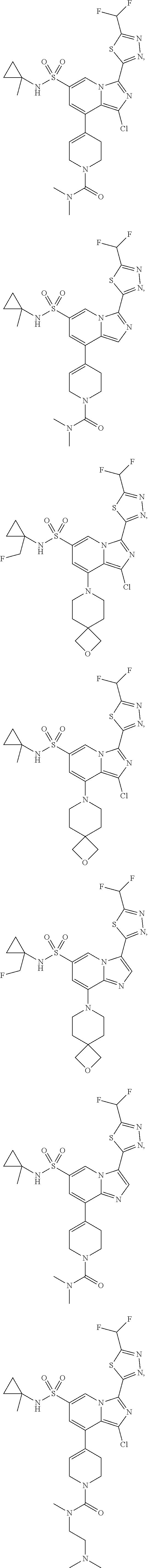

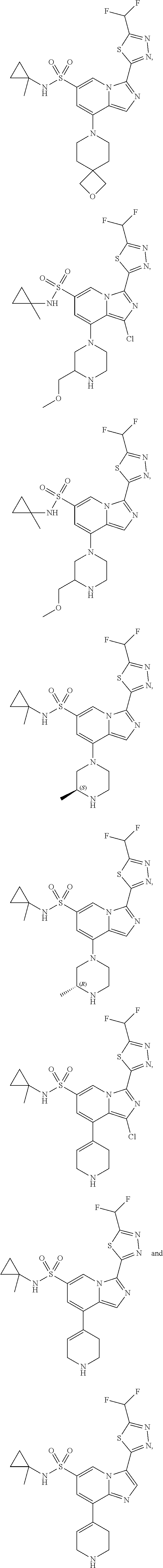

or a pharmaceutically acceptable salt, hydrate or solvate thereof.

The present invention also relates to each of the intermediates described further below in the examples section of this specification, including any one of these intermediates in non-salt form or in the form of a salt (e.g., a pharmaceutically acceptable salt) of the respective compound. Such intermediates can be used, in particular, in the synthesis of the compounds of formula (I).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The present invention further embraces the prodrugs of the compounds of formula (I). As preferably understood herein, the term “prodrug” of the compound of formula (I) refers to a derivative of the compounds of formula (I) that upon administration to a subject becomes metabolized to the said compound of formula (I). Said prodrugs of the compound of formula (I) may include modifications of —OH, —NH₂, or —COOH group if present in the compound of formula (I), which preferably can be hydrolyzed to —OH, —NH₂, or —COOH groups, respectively, e.g. upon administration to the subject. For example, as known to the skilled person, such prodrugs may preferably include for the compounds of formula (I) which comprise —OH moiety derivatives wherein said —OH moiety is turned into an —ORₓ moiety, wherein Rₓ preferably comprises a moiety selected from —CO—, —CH₂—O—CO, —CH₂—O—CO—O—, and —CH(CH₃)—O—COO—, more preferably wherein Rₓ is selected from —CO-Ry, —CH₂—O—CO—R_{y}, —CH₂—O—CO—O—R_{y}, and —CH(CH₃)—O—COO-Ry, wherein Ry is preferably carbocyclyl, heterocyclyl, C₁₋₅ alkyl, —NH—(C₁₋₅ alkyl) or —S—(C₁₋₅ alkyl), wherein the said alkyl is optionally substituted with a group selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein the said carbocyclyl and heterocyclyl are each optionally substituted with a group selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Furthermore, for example, as known to the skilled person, such prodrugs may preferably include for the compounds of formula (I) which comprise —NH₂ moiety derivatives wherein said —NH₂ moiety is turned into —NHCOO—R_{y} moiety, wherein R_{y} is as defined hereinabove. Furthermore, for examples, as known to the skilled person, such prodrugs may preferably include for the compounds of formula (I) which comprise —COOH moiety derivatives wherein said —COOH group is turned into —COOR_{y} moiety, wherein R_{y} is as defined hereinabove. Further examples of groups that can be derivatized to yield prodrugs are known to the skilled person.

### Pharmaceutical Compositions

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration. For each of the compounds or pharmaceutical compositions provided herein, it is particularly preferred that the respective compound or pharmaceutical composition is to be administered orally (particularly by oral ingestion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

### Therapeutic Use

In one embodiment, the present invention relates to the compound of formula (I), or a pharmaceutically acceptable salt, hydrate or solvate thereof, or a pharmaceutical composition as defined herein for use in therapy.

The present invention provides compounds that function as inhibitors of PARG. Thus, the present invention provides a method of inhibiting PARG enzyme activity in vitro or in vivo, said method comprising contacting a cell with an effective amount of the compound of formula (I), or a pharmaceutically acceptable salt, hydrate or solvate thereof, as defined herein.

The present invention also provides a method of selectively inhibiting PARG enzyme activity over PARP1 or ARH3 enzyme activity in vitro or in vivo. The said method comprises the steps of contacting a cell with an effective amount of a compound, or a pharmaceutically acceptable salt, hydrate or solvate thereof, as defined herein.

In a further embodiment, the present invention relates to the compound of formula (I), as disclosed herein, for use in a method of treating a disease or disorder in which PARG activity is implicated in a subject or patient in need of such treatment. Said method of treatment comprises administering to said subject/patient a therapeutically effective amount of a compound of formula (I), or a pharmaceutically acceptable salt, hydrate or solvate thereof, or a pharmaceutical composition as defined herein. In other words, in one embodiment the present invention relates to the compound of formula (I), as disclosed herein, for use in treating a disease or disorder in which PARG activity is implicated.

In a further embodiment, the present invention relates to a method of inhibiting cell proliferation, in vitro or in vivo, said method comprising contacting a cell with an effective amount of the compound of formula (I), or a pharmaceutically acceptable salt, hydrate or solvate thereof, as defined herein. Thus, the present invention relates to the compound of formula (I) or a pharmaceutically acceptable salt thereof for use in of inhibiting cell proliferation, in vitro or in vivo.

Thus, in a further embodiment, the present invention relates to a method of treating a proliferative disorder in a subject or patient in need of such treatment. The said method of treating a proliferative disorder in a subject or patient in need thereof comprises administering to said subject/patient a therapeutically effective amount of the compound of formula (I), or a pharmaceutically acceptable salt, hydrate or solvate thereof, or a pharmaceutical composition as defined herein. Preferably as disclosed herein, the proliferative disorder is cancer. Thus, the present invention relates to a method of treating cancer in a subject or patient in need thereof. The said method of treating cancer in a subject or patient in need thereof comprises administering to said subject/patient a therapeutically effective amount of the compound of formula (I), or a pharmaceutically acceptable salt, hydrate or solvate thereof, or a pharmaceutical composition as defined herein. In a particular embodiment, the cancer is human cancer.

In one embodiment, the present invention relates to the compound of formula (I) or a pharmaceutically acceptable salt, hydrate or solvate thereof, for use in treating a proliferative disorder. Preferably as disclosed herein, the proliferative disorder is cancer. Therefore, the present invention relates to the compound of formula (I) or a pharmaceutically acceptable salt, hydrate or solvate thereof for use in treating cancer. In a particular embodiment, the cancer is human cancer.

In a further embodiment, the present invention relates to the compound of formula (I), or a pharmaceutically acceptable salt, hydrate or solvate thereof, as defined herein, for use in the manufacture of a medicament for the treatment of a proliferative condition. In a preferred embodiment, the proliferative condition is cancer, more preferably a human cancer. Thus, preferably the present invention relates to the compound of formula (I), or a pharmaceutically acceptable salt, hydrate or solvate thereof, as defined herein, for use in the manufacture of a medicament for the treatment of cancer, preferably for the treatment of human cancer.

In a further embodiment, the present invention relates to the compound of formula (I), or a pharmaceutically acceptable salt, hydrate or solvate thereof, as defined herein, for use in the manufacture of a medicament for the inhibition of PARG enzyme activity. Preferably, the inhibition of PARG enzyme activity is selective inhibition of PARG enzyme activity over PARP1 or ARH3 enzyme activity. Thus, the present invention relates to the compound of formula (I), or a pharmaceutically acceptable salt, hydrate or solvate thereof, as defined herein, for use in the manufacture of a medicament for the selective inhibition of PARG enzyme activity over PARP1 or ARH3 enzyme activity.

The present invention further provides the compound of formula (I), or a pharmaceutically acceptable salt, hydrate or solvate thereof, as defined herein for use in the manufacture of a medicament for the treatment of a disease or disorder in which PARG activity is implicated, as defined herein.

As understood herein, the term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compound of formula (I) of the present invention have particular application in the treatment of human cancers (by virtue of their inhibition of PARG enzyme activity). The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The antiproliferative treatment with the compound of formula (I) or a pharmaceutically acceptable salt, hydrate or solvate thereof, as defined hereinbefore, may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5oc-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R1 15777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1 R kinase inhibitors, IGF receptor (insulin-like growth factor) kinase inhibitors; aurora kinase inhibitors (for example AZD1 152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU1 1248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213; (vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of formula (I) of the invention, conventional surgery or radiotherapy or chemotherapy. Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect the present invention further relates to the compound of formula (I) or a pharmaceutically acceptable salt, hydrate or solvate thereof, as defined herein, for use in the treatment of a cancer (for example a cancer involving a solid tumour) in combination with another anti-tumour agent. The anti-tumour agent is preferably selected from the anti-tumour agents as listed hereinabove.

As understood herein, the term “combination” refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

### EXAMPLES

The following examples are merely illustrative of the present invention and should not be construed to limit the scope of the invention which is defined by the appended claims.

### Synthesis of the Compounds of Formula (I)

The syntheses of embodiments A, B and C of the compounds of formula (I) according to the present invention are preferably carried out according to the general synthetic sequences as shown in Schemes 1-3.

In addition to said routes described below, also other routes may be used to synthesize the target compounds, in accordance with common general knowledge of a person skilled in the art of organic synthesis. The order of transformations exemplified in the following Schemes is therefore not intended to be limiting, and suitable synthesis steps from various schemes can be combined to form additional synthesis sequences. In addition, modification of any of the substituents can be achieved before and/or after the exemplified transformations. These modifications can be such as the introduction of protective groups, cleavage of protective groups, reduction or oxidation of functional groups, halogenation, metallation, metal-catalyzed coupling reactions, substitution or other reactions known to a person skilled in the art. These transformations include those which introduce a functionality allowing for further interconversion of substituents. Appropriate protective groups and their introduction and cleavage are well-known to a person skilled in the art (see for example: Greene's Protective Groups in Organic Synthesis; Editor: P.G.M. Wuts, 5th edition, Wiley 2014). Specific examples are described in the subsequent paragraphs. Further, it is possible that two or more successive steps may be performed without work-up being performed between said steps, e.g. a “one-pot” reaction, as it is well-known to a person skilled in the art. It is further understood to the skilled person that a reaction can lead to side product(s) which, when appropriate, can be used for the preparation of compounds of formula (I) using similar procedures as reported in the general schemes hereinbelow.

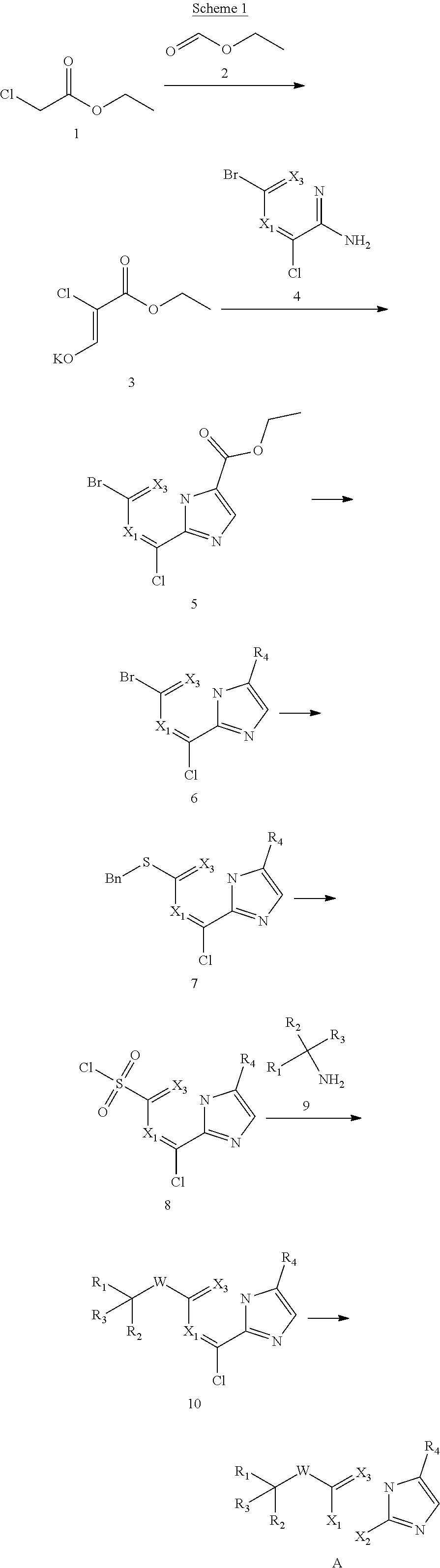

Scheme 1 illustrates a preferred synthetic approach to compounds of the general formula A. As it is to be understandable to the skilled person, the scheme can also be extended to the compounds of formula (I) wherein X₄ is N and X₅ is C—R_{C5}, for example upon functionalization of C—H of compound A to C—R_{C5} through bromination of the C—H position (see for example: Kim et al, KR2012078530) followed by palladium-catalyzed cross-coupling reactions.

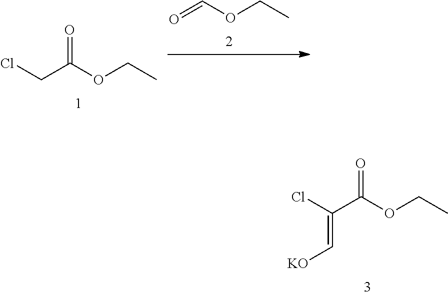

In the first step, ethyl 2-chloroacetate 1 is reacted with ethyl formate 2 under basic condition to provide potassium (Z)-2-chloro-3-ethoxy-3-oxoprop-1-en-1-olate 3. The reaction is preferably carried out in solvents like tert-butyl methyl ether, di-isopropyl ether, diethyl ether, 1,2-dimethoxyethane, dioxane, DMF, DME, THF, or a mixture of toluene, diethyl ether, and EtOH in the presence of a base like sodium ethoxide, sodium methoxide, potassium tert-butylate or sodium tert-butylate. (see for examples: a) Stephen et al, US2017/369489; b) Murar et al, Eu. J. Med. Chem. 2017, 126, 754). The reaction is performed at temperatures ranging from −78° C. to the room temperature. The reaction is preferably completed after 1-24 hours.

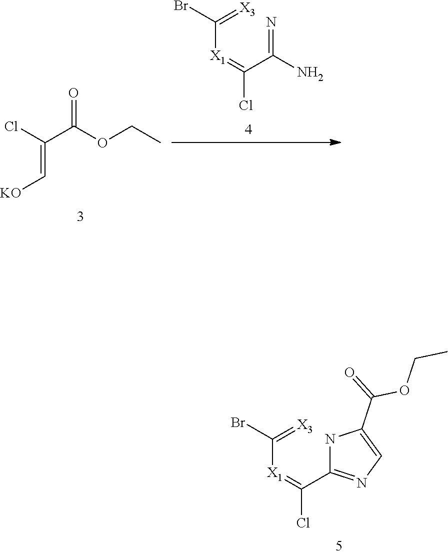

In the second step, a compound of formula 4, in which X₁ and X₃ are as defined for the compound of formula (I), is reacted with potassium (Z)-2-chloro-3-ethoxy-3-oxoprop-1-en-1-olate 3 to give a compound of formula 5. This cyclization can be carried out under acidic conditions (see for example: X₁ et al, WO2019/99311). Preferred is the herein described use of sulfuric acid in EtOH. The reactions are preferably run for 5-24 hours at 70-100° C.

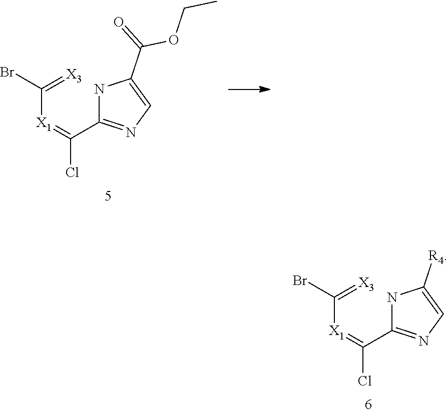

In the third step, a compound of formula 5 in which X₁ and X₃ are as defined for the compound of formula (I) is converted to a compound of formula 6 in which X₁, X₃ and R₄ is as defined for the compound of formula (I) in several synthetic steps. If R₄ is a 2-(difluoromethyl)-1,3,4-thiadiazole group, a compound of formula 5 is reacted with hydrazine hydrate to produce a hydrazide. This hydrazide formation can be carried out under neutral condition. (see for example: Dong et al, J. Med. Chem. 2020, 63, 3028). The hydrazide formation is preferably performed in EtOH and the reactions are preferably run for 1-24 hours at 50-100° C. with heating or microwave conditions. The hydrazide is then reacted with ethyl 2,2-difluoroacetate to produce a di-acyl hydrazine. This reaction can be carried out under basic condition, preferred is the herein described use of DBU in EtOH, THF, or DMF. The reactions are preferably run for 0.5-24 hours at room temperature to 100° C. in a microwave oven or in an oil bath. Finally, the di-acyl hydrazine is cyclized by treatment with oxygen/sulfur exchange reagents to a compound of formula 6, in which R₄ is 2-(difluoromethyl)-1,3,4-thiadiazole group. (see for example: Brunet et al, WO2020/127974). Preferred is the herein described use of Lawessons reagent in toluene or THF. The reactions are preferably run for 0.5-24 hours at 50-130° C.

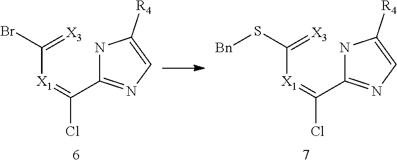

In the fourth step, a compound of formula 6 in which X₁, X₃ and R₄ are as defined for the compound of formula (I) is reacted with benzyl mercaptan to give a compound of formula 7. This coupling reaction can be carried out by a palladium-catalyzed C—S cross-coupling reaction (see for example: Jiang, Buchwald in ‘Metal-Catalyzed Cross-Coupling Reactions’, 2^{nd} edition: de Meijere, Diederich, Eds.: Wiley-VCH: Weinheim, Germany, 2004). Preferred is the herein described use of tris(dibenzylideneacetone) dipalladium(0), (9,9-dimethyl-9H-xanthene-4,5-diyl)bis(diphenylphosphane) and N-ethyl-N-isopropylpropan-2-amine in dioxane. The reactions are preferably run under an atmosphere of argon for 1-48 hours at 80-100° C. in a microwave oven or in an oil bath.

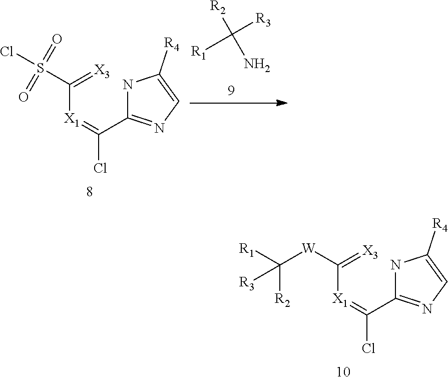

In the fifth step, a compound of formula 7 in which X₁, X₃ and R₄ are as defined for the compound of formula (I) is reacted with chlorination reagent to give a sulfonyl chloride of formula 8. This sulfonyl chloride formation can be carried out by treatment with NCS, sulfonyl chloride, DCDMH, Cl₂ etc., in MeCN with equivalent acetic acid and water. (see for example: Sutton et al, WO 2021/055744). Preferred is the herein described use of DCDMH in MeCN with equivalent acetic acid and water. The reactions are preferably run under an atmosphere of argon for 0.5-5 hours at 0° C. to room temperature.

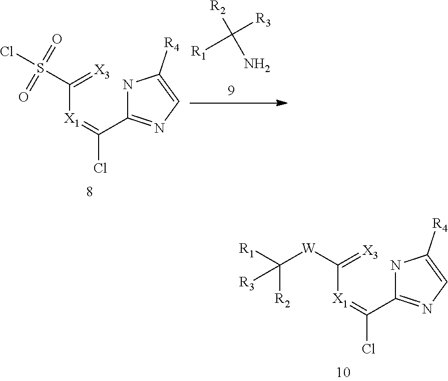

In the sixth step, a compound of formula 8 in which X₁, X₃ and R₄ are as defined for the compound of formula (I) is reacted with an amine of formula 9 in which R₁, R₂ and R₃ are as defined for the compound of formula (I) to give a compound of formula 10. This reaction can be carried out under basic conditions (see for example: Sutton et al, WO 2021/055744). Preferred is the herein described use of trimethylamine, pyridine etc., in DCM, THE or DMF. The reactions are preferably run under an atmosphere of argon for 0.5-24 hours at 0° C. to room temperature.

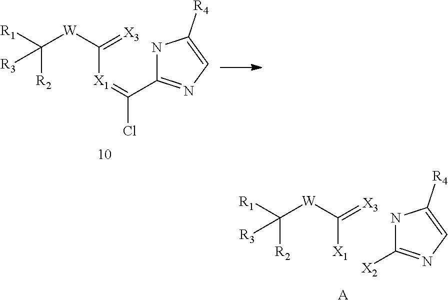

In the final step, a compound of formula 10 in which X₁, X₃, R₁, R₂, R₃ and R₄ are as defined for the compound of formula (I) is coupled with various amines to give a compound of formula A, in which X₂ is defined as for the compound of formula (I). This coupling reaction can be carried out by a palladium-catalyzed C—N cross-coupling reaction (see for example: a) Jiang, Buchwald in ‘Metal-Catalyzed Cross-Coupling Reactions’, 2^{nd} edition: de Meijere, Diederich, Eds.: Wiley-VCH: Weinheim, Germany, 2004; b) Sutton, et al, WO 2021/055744). Preferred is the herein described use of cesium carbonate and Pd-PEPPSI-IHept Cl in dioxane. The reactions are preferably run under an atmosphere of argon for 1-48 hours at 80-120° C. in a microwave oven or in an oil bath. Preferred is also the herein described use of cesium carbonate RuPhos-Pd-G3, Ruphos in dioxane or palladium acetate, Ruphos, tert-butyl alcohol sodium in THE. The reactions are preferably run under an atmosphere of argon for 1-24 hours at 70-130° C. in a microwave oven or in an oil bath.

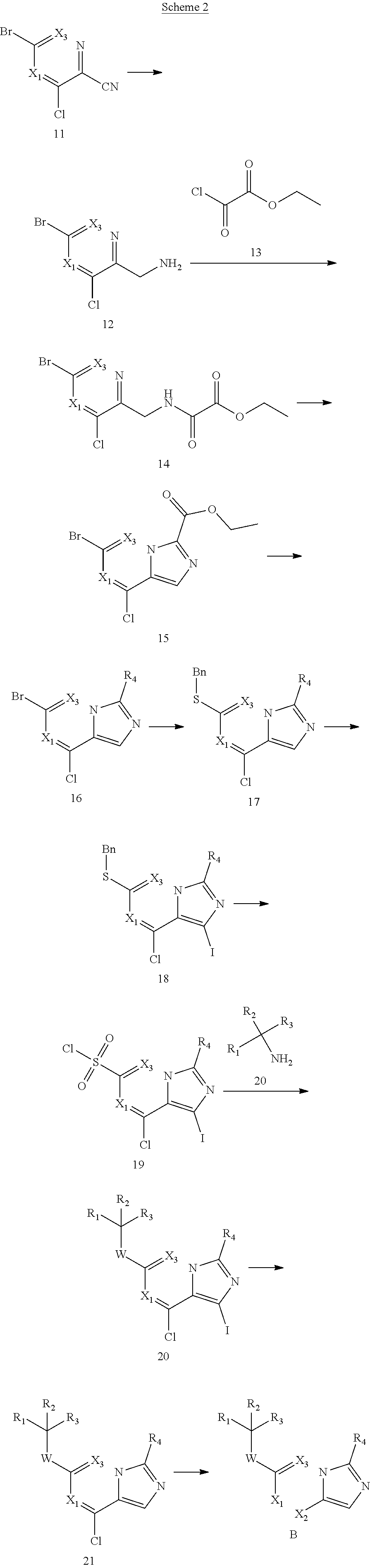

Scheme 2 illustrates a preferred synthetic approach to compounds of the general formula B. As it is to be understandable to the skilled person, the compounds of formula (I) wherein X₄ is C—R_{C4} and X₅ is N are obtainable through functionalization of the C-I position in compound 20, e.g. via palladium-catalyzed cross-coupling reactions.

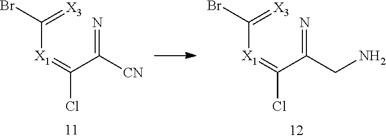

In the first step, the cyano group of a compound of formula 11, in which X₁ and X₃, are as defined for the compound of formula (I) is reduced to give a compound of formula 12. The reaction is preferably carried out in THE in the presence of a reducing agent like BH₃·THF, BH₃·Me₂S, PtO₂/H₂, sodium tetrahydroborate etc., (see for example: Long et al, WO2018/71535). The reaction is performed at temperatures ranging from 20-40° C. The reaction is preferably completed after 0.5-24 hours.

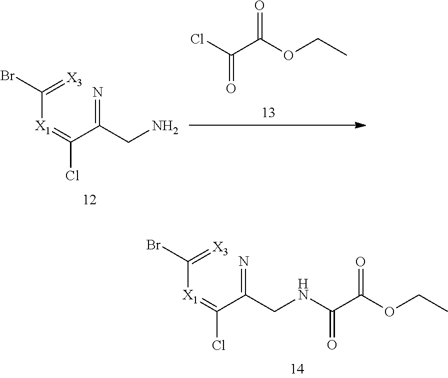

In the second step, a compound of formula 12 in which X₁ and X₃, are as defined for the compound of formula (I) is reacted with ethyl 2-chloro-2-oxoacetate 13 under basic condition to give a compound of formula 14. The acylation is preferably carried out in a solvent like DCM, dioxane or THF, in the presence of a base like trimethylamine or N-ethyl-N-isopropylpropan-2-amine (see for example: Blaquiere et al, WO2015/25025). The reaction is performed at temperatures ranging from −5° C. to room temperature. The reaction is preferably completed after 1-24 hours.

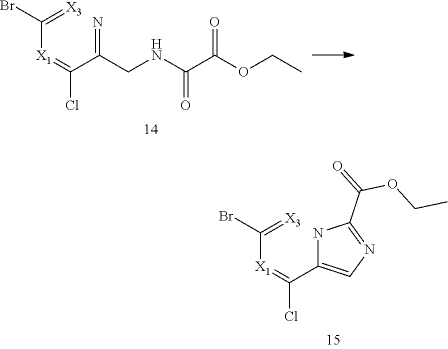

In the third step, a compound of formula 14 in which X₁ and X₃ are as defined for the compound of formula (I) is converted to a compound of formula 15. The cyclization is preferably carried out in the presence of dehydration reagents like trichlorophosphate, phosphorus pentoxide and trichlorophosphate, pyridine and trifluoroacetic anhydride etc., in 1,2-dichloro-ethane, toluene or neat conditions. The reaction is performed at temperatures ranging from 70-140° C. The reaction is preferably completed after 1-24 hours.

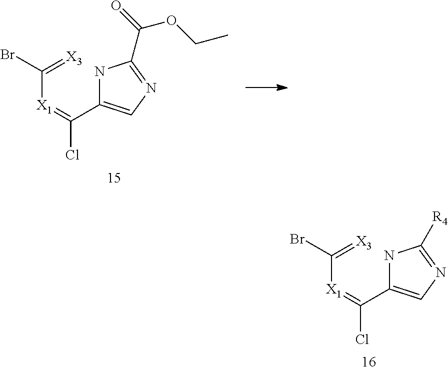

In the fourth step, a compound of formula 15 in which X₁ and X₃ are as defined for the compound of formula (I) is converted to a compound of formula 16 by several synthetic steps. If R₄ is 2-(difluoromethyl)-1,3,4-thiadiazole, a compound of formula 15 is reacted with hydrazine hydrate to produce a hydrazide. This hydrazide formation can be carried out under neutral conditions (see for example: Dong et al, J. Med. Chem. 2020, 63, 3028). The hydrazide formation is preferably performed in EtOH and the reactions are preferably run for 1-24 hours at 50-100° C. with heating or microwave conditions. The hydrazide is then reacted with ethyl 2,2-difluoroacetate to produce a di-acyl hydrazine. This reaction can be carried out by basic condition, preferred is the herein described use of DBU in EtOH, THF, or DMF. The reactions are preferably run for 0.5-24 hours at room temperature to 100° C. in a microwave oven or in an oil bath. Finally, the di-acyl hydrazine is cyclized by treatment with oxygen/sulfur exchange reagents to a compound of formula 16, in which R₄ is 2-(difluoromethyl)-1,3,4-thiadiazole group. (see for example: Brunet et al, WO2020/127974). Preferred is the herein described use of Lawessons reagent in toluene or THF. The reactions are preferably run for 0.5-24 hours at 50-130° C.

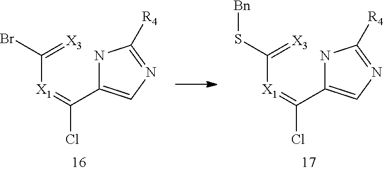

In the fifth step, a compound of formula 16 in which X₁, X₃ and R₄ are as defined for the compound of formula (I) is reacted with benzyl mercaptan to give a compound of formula 17. This coupling reaction can be carried out by a palladium-catalyzed C—S cross-coupling reaction (see for example: Jiang, Buchwald in ‘Metal-Catalyzed Cross-Coupling Reactions’, 2^{nd} edition: de Meijere, Diederich, Eds.: Wiley-VCH: Weinheim, Germany, 2004). Preferred is the herein described use of tris(dibenzylideneacetone) dipalladium(0), (9,9-dimethyl-9H-xanthene-4,5-diyl)bis(diphenylphosphane) and N-ethyl-N-isopropylpropan-2-amine in dioxane. The reactions are preferably run under an atmosphere of argon for 1-48 hours at 80-100° C. in a microwave oven or in an oil bath.

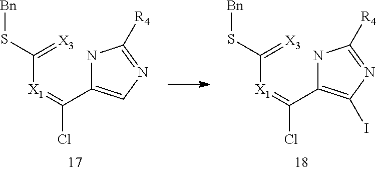

In the sixth step, a compound of formula 17 in which X₁, X₃ and R₄ are as defined for the compound of formula (I) is reacted with an iodide reagent to give a compound of formula 18. This iodization can be carried out by treatment with NIS, 12 etc., in MeCN, THF, dioxane, DMF etc. (see for example: Bentley et al; WO2011/138266). Preferred is the herein described use of NIS in MeCN. The reactions are preferably run under an atmosphere of argon for 0.5-5 hours at 0° C. to room temperature.

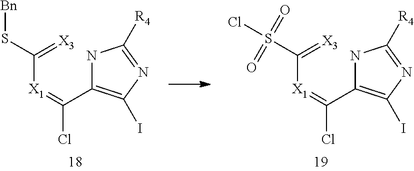

In the seventh step, a compound of formula 18 in which X₁, X₃ and R₄ are as defined for the compound of formula (I) is reacted with chlorination reagent to give a sulfonyl chloride of formula 19. This sulfonyl chloride formation can be carried out by treatment with NCS, sulfonyl chloride, DCDMH, Cl₂ etc., in MeCN with equivalent acetic acid and water. (see for example: Sutton et al, WO 2021/055744). Preferred is the herein described use of DCDMH in MeCN with equivalent acetic acid and water. The reactions are preferably run under an atmosphere of argon for 0.5-5 hours at 0° C. to room temperature.

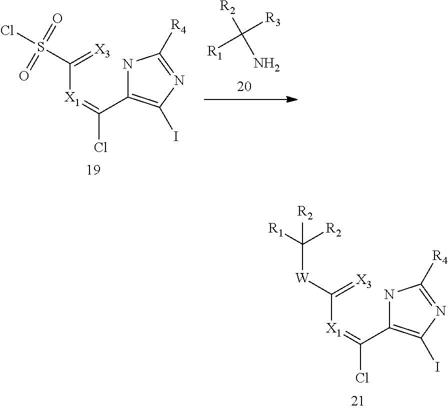

In the eighth step, a compound of formula 19 in which X₁, X₃ and R₄ are as defined for the compound of formula (I) is reacted with an amine of formula 20 in which R₁, R₂ and R₃ are as defined for the compound of formula (I) to give a compound of formula 21. This reaction can be carried out under basic conditions (see for example: Sutton et al, WO 2021/055744). Preferred is the herein described use of trimethylamine, pyridine etc., in DCM, THF or DMF. The reactions are preferably run under an atmosphere of argon for 0.5-24 hours at 0° C. to room temperature.

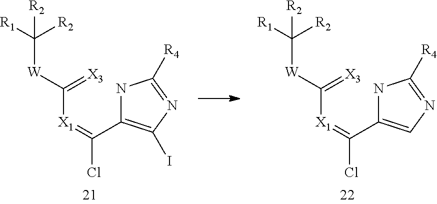

In the ninth step, the iodide of a compound of formula 21 in which X₁, X₃ and R₄ are as defined for the compound of formula (I) is removed by hydrogenation to give a compound of formula 22. The reaction is preferably carried out in THF, MeOH, EtOH, dioxane or DMF in the presence of a hydrogenation catalyst like Pd/C, Pd(OH)₂, Raney Ni, PtO₂ etc. under an atmosphere of hydrogen (see for example: Aissaoui et al, US2011/105514). The reaction is performed at temperatures ranging from 20-80°. The reaction is preferably completed after 0.5-24 hours.

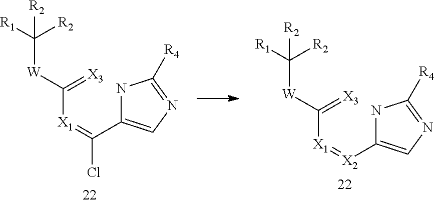

In the final step, a compound of formula 22 in which X₁, X₃, R₁, R₂, R₃ and R₄ are as defined for the compound of formula (I) is coupled with various amines to give a compound of formula B, in which X₂ is defined as for the compound of formula (I). This coupling reaction can be carried out by a palladium-catalyzed C—N cross-coupling reaction (see for example: a) Jiang, Buchwald in ‘Metal-Catalyzed Cross-Coupling Reactions’, 2^{nd} edition: de Meijere, Diederich, Eds.: Wiley-VCH: Weinheim, Germany, 2004; b) Sutton et al, WO 2021/055744). Preferred is the herein described use of cesium carbonate and Pd-PEPPSI-IHept Cl in dioxane. The reactions are preferably run under an atmosphere of argon for 1-48 hours at 80-120° C. in a microwave oven or in an oil bath. Preferred is also the herein described use of cesium carbonate RuPhos-Pd-G3, Ruphos in dioxane or palladium acetate, Ruphos, tert-butyl alcohol sodium in THF. The reactions are preferably run under an atmosphere of argon for 1-24 hours at 70-130° C. in a microwave oven or in an oil bath.

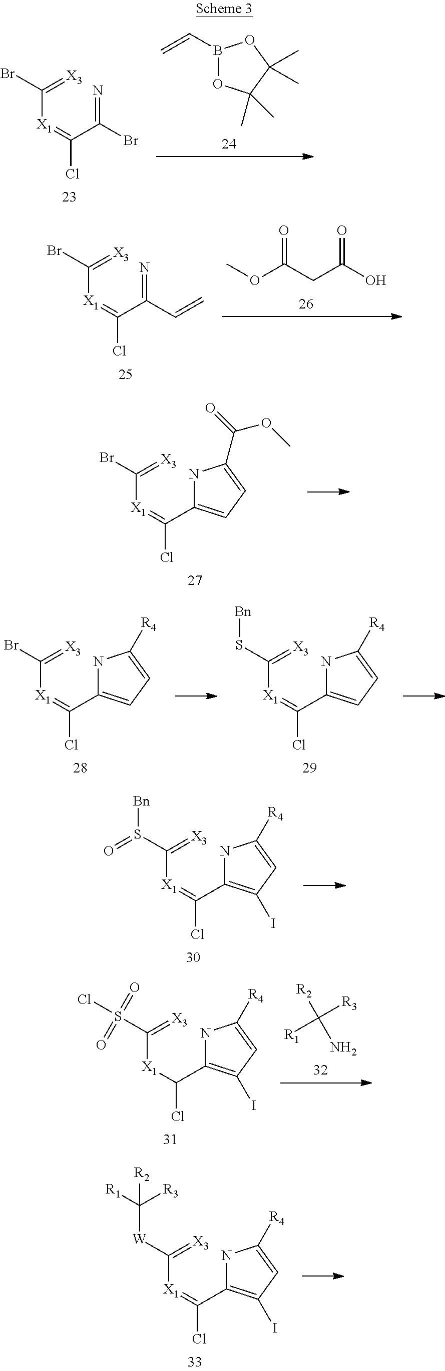

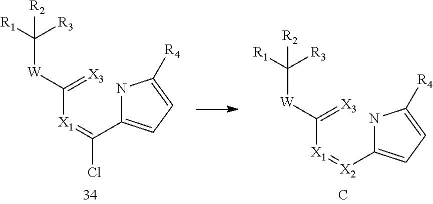

Scheme 3 illustrates a preferred synthetic approach to the compounds of the general formula C. As it is to be understandable to the skilled person, the compounds of formula (I) wherein X₄ is C—R_{C4} are obtainable through functionalization of C-I position of compound 33, e.g. via palladium-catalyzed cross-coupling reactions. As it is to be understandable to the skilled person, the compounds of formula (I) wherein X₅ is C—R_{C5} are obtainable through functionalization of the X₅ is CH position through bromination of the C—H position (see for example: Yao et al, Org. Lett. 2020, 22, 4511) followed by palladium-catalyzed cross-coupling reactions.

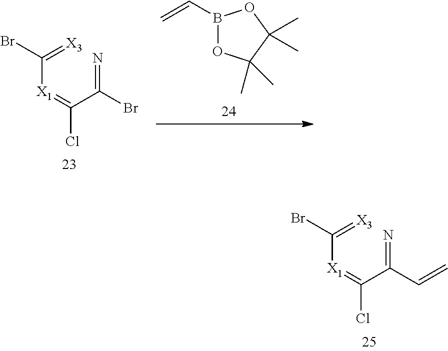

In the first step a compound of formula 23 in which X₁ and X₃, are as defined for the compound of formula (I) is reacted with 4,4,5,5-tetramethyl-2-vinyl-1,3,2-dioxaborolane 24 to give a compound of formula 25. The coupling reaction is catalyzed by palladium catalysts, e.g. by Pd(0) catalysts like tetrakis(triphenylphosphine) palladium(0) [Pd(PPh₃)₄], tris(dibenzylideneacetone) di-palladium(0) [Pd₂(dba)₃], or by Pd(II) catalysts like dichlorobis(triphenylphosphine)-palladium(II) [Pd(PPh₃)₂Cl₂], palladium(II) acetate and triphenylphosphine or by [I,I′-bis(diphenylphosphino)ferrocene]palladium dichloride. The reaction is preferably carried out in a solvent like 1,2-dimethoxyethane, dioxane, DMF, DME, THF, or isopropanol with water and in the presence of a base like potassium carbonate, sodium carbonate, sodium bicarbonate or potassium phosphate. (see for example: Hall, Boronic Acids, 2005 Wiley VCH Verlag GmbH & Co. KGaA, Weinheim, ISBN 3-527-30991-8 and references cited therein). The reaction is performed at temperatures ranging from room temperature to the boiling point of the respective solvent. Further on, the reaction can be performed at temperatures above the boiling point using pressure tubes and a microwave oven. The reaction is preferably completed after 1 to 36 hours.

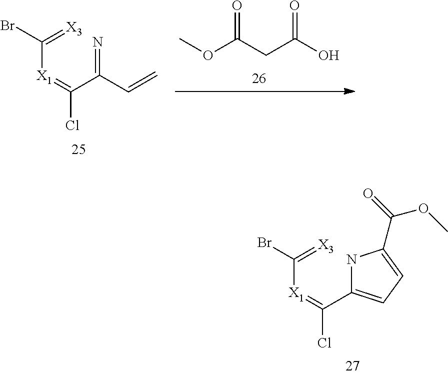

In the second step, a compound of formula 25 in which X₁ and X₃, are as defined for the compound of formula (I) is reacted with 3-methoxy-3-oxopropanoic acid 26 to give a compound of formula 27. The cyclization is preferably carried out in a solvent like 1,2-dimethoxyethane, dioxane, DMF, DME, THF, or MeCN in the presence of N-iodo-succinimide and sodium acetate. (see for example: Tang et al, Adv. Synth. Catalysis, 2016, 358, 2878). The reaction is performed at temperatures ranging from 80-100° C. in a microwave oven or in an oil bath. The reaction is preferably completed after 1 to 36 hours.

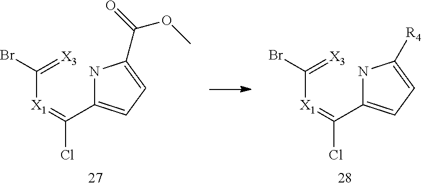

In the third step, a compound of formula 27 in which X₁ and X₃ are as defined for the compound of formula (I) is converted to a compound of formula 28 by several synthetic steps. If R₄ is 2-(difluoromethyl)-1,3,4-thiadiazole, a compound of formula 27 is reacted with hydrazine hydrate to produce a hydrazide. This hydrazide formation can be carried out under neutral conditions (see for example: Dong et al, J. Med. Chem. 2020, 63, 3028). The hydrazide formation is preferably performed in EtOH and the reactions are preferably run for 1-24 hours at 50-100° C. with heating or microwave conditions. The hydrazide is then reacted with ethyl 2,2-difluoroacetate to produce a di-acyl hydrazine. This reaction can be carried out under basic conditions, preferred is the herein described use of DBU in EtOH, THF, or DMF. The reactions are preferably run for 0.5-24 hours at room temperature to 100° C. in a microwave oven or in an oil bath. Finally, the di-acyl hydrazine is cyclized by treatment with oxygen/sulfur exchange reagents to a compound of formula 28, in which R₄ is 2-(difluoromethyl)-1,3,4-thiadiazole group. (see for example: Brunet et al, WO2020/127974). Preferred is the herein described use of Lawessons reagent in toluene or THF. The reactions are preferably run for 0.5-24 hours at 50-130° C.

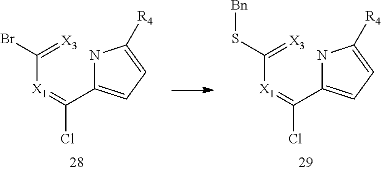

In the fourth step, a compound of formula 28 in which X₁, X₃ and R₄ are as defined for the compound of formula (I) is reacted with benzyl mercaptan to give a compound of formula 29. This coupling reaction can be carried out by a palladium-catalyzed C—S cross-coupling reaction (see for example: Jiang, Buchwald in ‘Metal-Catalyzed Cross-Coupling Reactions’, 2^{nd} edition: de Meijere, Diederich, Eds.: Wiley-VCH: Weinheim, Germany, 2004). Preferred is the herein described use of tris(dibenzylideneacetone) dipalladium(0), (9,9-dimethyl-9H-xanthene-4,5-diyl)bis(diphenylphosphane) and N-ethyl-N-isopropylpropan-2-amine in dioxane. The reactions are preferably run under an atmosphere of argon for 1-48 hours at 80-100° C. in a microwave oven or in an oil bath.

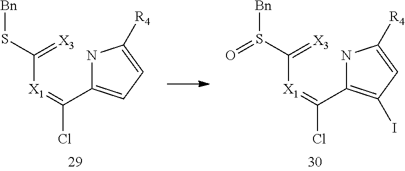

In the fifth step, a compound of formula 29 in which X₁, X₃ and R₄ are as defined for the compound of formula (I) is reacted with an iodide reagent to give a compound of formula 30. This iodization can be carried out by treatment with NIS, 12 etc., in MeCN, THF, dioxane, DMF etc. (see for example: Bentley et al, WO2011/138266). Preferred is the herein described use of NIS in MeCN. The reactions are preferably run under an atmosphere of argon for 0.5-5 hours at 0° C. to room temperature.

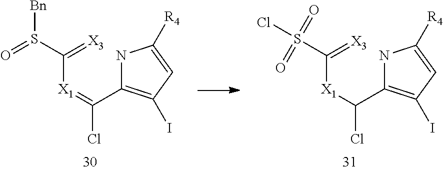

In the six step, a compound of formula 30 in which X₁, X₃ and R₄ are as defined for the compound of formula (I) is reacted with chlorination reagent to give a sulfonyl chloride of formula 31. This sulfonyl chloride formation can be carried out by treatment with NCS, sulfonyl chloride, DCDMH, Cl₂ etc., in MeCN with equivalent acetic acid and water. (see for example: Sutton et al, WO 2021/055744). Preferred is the herein described use of DCDMH in MeCN with equivalent acetic acid and water. The reactions are preferably run under an atmosphere of argon for 0.5-5 hours at 0° C. to room temperature.

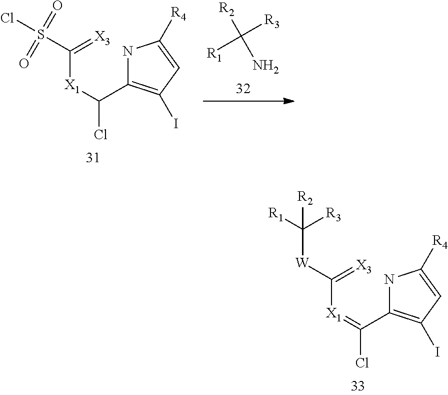

In the seventh step, a compound of formula 31 in which X₁, X₃ and R₄ are as defined for the compound of formula (I) is reacted with an amine of formula 32 in which R₁, R₂ and R₃ are as defined for the compound of formula (I) to give a compound of formula 33. This reaction can be carried out under basic conditions (see for example: Sutton et al, WO 2021/055744). Preferred is the herein described use of trimethylamine, pyridine etc., in DCM, THE or DMF. The reactions are preferably run under an atmosphere of argon for 0.5-24 hours at 0° C. to room temperature.

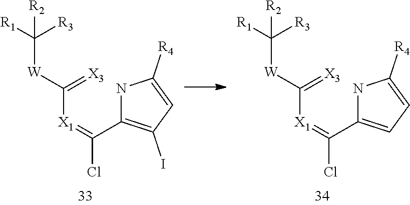

In the eighth step, the iodide of a compound of formula 33 in which X₁, X₃ and R₄ are as defined for the compound of formula (I) is removed by hydrogenation to give a compound of formula 34. The reaction is preferably carried out in THF, MeOH, EtOH, dioxane or DMF in the presence of a hydrogenation catalyst like Pd/C, Pd(OH)₂, Raney Ni, PtO₂ etc. under an atmosphere of hydrogen, (see for example: Aissaoui et al, US2011/105514). The reaction is performed at temperatures ranging from 20-80°. The reaction is preferably completed after 0.5-24 hours.

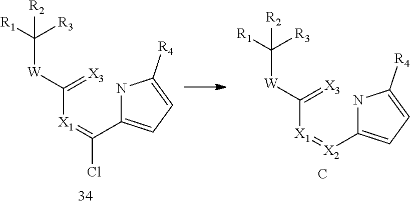

In the final step, a compound of formula 34 in which X₁, X₃, R₁, R₂, R₃ and R₄ are as defined for the compound of formula (I) is coupled with various amines to give a compound of formula (C, in which X₂ is defined as for the compound of formula (I). This coupling reaction can be carried out by a palladium-catalyzed C—N cross-coupling reaction (see for example: a) Jiang, Buchwald in ‘Metal-Catalyzed Cross-Coupling Reactions’, 2^{nd} edition: de Meijere, Diederich, Eds.: Wiley-VCH: Weinheim, Germany, 2004; b) Sutton et al, WO 2021/055744). Preferred is the herein described use of cesium carbonate and Pd-PEPPSI-IHept Cl in dioxane. The reactions are preferably run under an atmosphere of argon for 1-48 hours at 80-120° C. in a microwave oven or in an oil bath. Preferred is also the herein described use of cesium carbonate RuPhos-Pd-G3, Ruphos in dioxane or palladium acetate, Ruphos, tert-butyl alcohol sodium in THE. The reactions are preferably run under an atmosphere of argon for 1-24 hours at 70-130° C. in a microwave oven or in an oil bath.

### Preparative Examples

### General Considerations

Abbreviations used in the descriptions that follow are: AcOH (acetic acid); aq. (aqueous); Ar (Argon); Atm (atmosphere); BH₃·THF (boran tetrahydrofuran complex); br. (broad, ¹H NMR signal); Boc₂O (di-tert-butyldicarbonate); (Cataxium APdG₃ (Mesylate[(di(1-adamantyl)-n-butylphosphine)-2-(2′-amino-1,1′-biphenyl)]palladium(II)); (CDCl₃ (deuterated chloroform); cHex (cyclohexane); CMPB (Cyanomethylene trimethylphosphorane); Cs₂CO₃ (cesium carbonate); CuI (copper iodide); DABCO ((1,4-diazabicyclo[2.2.2]octane)); DAST (diethylaminosulfur trifluoride); DBU (1,8-Diazabicyclo(5.4.0)undec-7-ene); DCE (dichloroethane); d (doublet, ¹H NMR signal); DCM (dichloromethane); DIBAL-H (diisobutyl aluminium hydride); DIPEA or DIEA (di-iso-propylethylamine); DMAP (4-N—N-dimethylaminopyridine), DME (1,2-dimethoxyethane), DMEDA (dimethylethylenediamine); DMF (N—N-dimethylformamide); DMSO (dimethyl sulfoxide); DPPA (diphenylphosphoride azide); dtbbpy (Bis(1,1-dimethylethyl)-2,2′-bipyridine); ES (electrospray); EtOAc or EA (ethyl acetate); EtOH (ethanol); h (hour(s)); FA (formic acid); HATU (1-[Bis(dimethylamino)methylene]-1H-1,2,3-triazolo[4,5-b]pyridinium 3-oxide hexafluorophosphate); HFIP (Hexafluoroisopropanol); 1H NMR (proton nuclear magnetic resonance spectroscopy); HPLC (High Performance Liquid Chromatography), iPrOH (iso-propanol); K₃PO₄ (tripotassium phosphate); Ir[dF(CF₃)(dtbbpy)PF₆ ((4,4′-Di-t-butyl-2,2′-bipyridine)bis[3,5-difluoro-2-[5-trifluoromethyl-2-pyridinyl-kN)phenyl-kC]iridium(III) hexafluorophosphate); LiOH (lithium hydroxide); m (multiplet, ¹H NMR signal); mCPBA (meta-chloroperoxybenzoic acid), MeCN (acetonitrile), MeOH (methanol); min (minute(s)); MnO₂ (Manganese (IV) oxide); MS (mass spectrometry); MTBE (methyl tert-butyl ether); NaBH₄ (sodium borohydride); NaHCO₃ (sodium hydrogenocarbonate); Na₂S₂O₃ (sodium thiosulfate); NCS (N-chlorosuccinimide); NH₃ (ammonia); NH₄Cl (ammonium fluoride); NiCl₂ (nickel dichloride); NIS (N-Iodosuccinimide); NMP (N-methylpyrrolidone); NMR (nuclear magnetic resonance); Pd/C (palladium on charcoal); Pd₂dba₃ (tris(dibenzylideneacetone)dipalladium); Pd(dppf)Cl₂ (1,1-Bis(diphenylphosphino)ferrocene dichloropalladium); Pd(Ph₃)₂Cl₂ (Bis(triphenylphosphine)palladium(II) dichloride); PE (petroleum ether); Pd-PEPPSI-IPentCl o-picoline ([1,3-bis[2,6-bis(1-ethylpropyl)phenyl]-4,5-dichloro-imidazol-2-ylidene]-dichloro-(2-methylpyridin-1-ium-1-yl)palladium; Pd(OH)₂ (palladium hydroxide); Pd(Ph₃)₄ (Palladium-tetrakis(triphenylphosphine)); PhI(OAc)₂ ((Diacetoxyiodo)benzene)); P(tBu)₃ (Tri-tert-butylphosphine); Py (pyridine); q (quartet, 1H NMR signal); quin (quintet, 1H NMR signal); rac (racemic); RT (retention time); s (singlet, ¹H NMR signal); sat. (saturated); t (triplet, ¹H NMR signal); TBAF (tetrabutylammonium fluoride); tert-BuBrettPhos-Pd-G3 ([(2-Di-tert-butylphosphino-3,6-dimethoxy-2′,4′,6′-triisopropyl-1,1′-biphenyl)-2-(2′-amino-1,1′-biphenyl)]palladium(II) methanesulfonate); tBuXPhos Pd G3 (Methanesulfonato(2-di-t-butylphosphino-2′,4′,6′-tri-i-propyl-1,1′-biphenyl)(2′-amino-1,1′-biphenyl-2-yl)palladium(II))TBDMSCl or TBSCl (tert-butyldimethylsilyl chloride); tBuOH (tert-butanol); TEA (triethylamine); TFA (trifluoroacetic acid); TFAA (trifluoroacetic anhydride), THE (tetrahydrofuran); TLC (thin layer chromatography); TMSCHN2 (Trimethylsilyldiazomethane); TMSCN (trimethylsilyl cyanide); TMSOTf (Trimethylsilyl trifluoromethanesulfonate); TTMSS (trimethylsilane); UPLC (Ultra-High Performance Liquid Chromatography), UV (ultraviolet), wt-% (percent by weight); Xantphos (4,5-Bis(diphenylphosphino)-9,9-dimethylxanthene); Xantphos Pd G4 (Methanesulfonato[9,9-dimethyl-4,5-bis(diphenylphosphino)xanthene](2′-methylamino-1,1′-biphenyl-2-yl)palladium(II)).

General Procedure: All starting materials and solvents were obtained either from commercial sources or prepared according to literature references. Commercially available reagents and anhydrous solvents were used as supplied, without further purification. Unless otherwise stated all reactions were stirred. Organic solutions were routinely dried over anhydrous sodium sulfate. Column chromatography was performed on pre-packed silica (100-1000 mesh, 40-63 μm) cartridges using the amount indicated. All air- and moisture-sensitive reactions were carried out in oven-dried (at 120° C.) glassware under an inert atmosphere of nitrogen or argon. Compound names were generated using ChemDraw Prime (Perkin Elmer). In some cases generally accepted names of commercially available reagents were used in place of ChemDraw generated names.

### Reversed Phase HPLC Conditions for LCMS Analysis of Final Compounds:

Method 1: SHIMADZU LCMS-2020 Kinetex EVO C18 2.1×30 mm, 5 μm at 50° C.; Mobile Phase: A: 0.0375% TFA in water (v/v); B: 0.01875% TFA in MeCN (v/v); flow rate held at 1.5 mL/min; eluted with the mobile phase over 1.55 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

Method 2: SHIMADZU LCMS-2020 Kinetex EVO C18 2.1×30 mm, 5 μm at 40° C.; Mobile Phase A: 0.025% NH₃·H₂O in water (v/v), B: MeCN; flow rate held at 1.5 mL/min; eluted with the mobile phase over 1.55 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

Method 3: SHIMADZU LCMS-2020 Kinetex EVO C18 2.1×30 mm, 5 μm at 50° C.; Mobile Phase: A: 0.0375% TFA in water (v/v); B: 0.01875% TFA in MeCN (v/v); flow rate held at 2.0 mL/min; eluted with the mobile phase over 0.80 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

Method 4: SHIMADZU LCMS-2020 Kinetex® EVO C18 2.1×20 mm 2.6 um at 50° C.; Mobile Phase: A: 0.0375% TFA in water (v/v); B: 0.01875% TFA in MeCN (v/v); flow rate held at 2.0 mL/min; eluted with the mobile phase over 1.00 min employing UV detection at 220 nm and 254 nm. Gradient information: 0.01-0.60 min, ramped from 95% A-5% B to 5% A-95% B; 0.61-0.78 min, held at 5% A-95% B; 0.78-0.79 min, returned to 95% A-5% B, 0.79-0.80 min, held at 95% A-5% B.

Method 5: SHIMADZU LCMS-2020 Kinetex EVO C18 2.1×30 mm, 5 μm at 50° C.; Mobile Phase: A: 0.0375% TFA in water (v/v); B: 0.01875% TFA in MeCN (v/v); flow rate held at 2.0 mL/min; eluted with the mobile phase over 0.80 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

### ¹H NMR Spectroscopy:

¹H NMR spectra were acquired on a Bruker Avance III spectrometer at 400 MHz using residual undeuterated solvent as reference. ¹H NMR signals are specified with their multiplicity/combined multiplicities as apparent from the spectrum; possible higher-order effects are not considered. Chemical shifts of the signals (6) are specified as ppm (parts per million).

### Salt Stoichiometry:

In the present text, in particular in the experimental section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae such as “hydrochloride”, “trifluoroacetate”, “sodium salt”, or “x HO”, “x CF3COOH”, “x Na+”, for example, are to be understood as not a stoichiometric specification, but solely as a salt form. This applies analogously to cases in which synthesis intermediates or example compounds or salts thereof have been obtained, by the preparation and/or purification processes described, as solvates, such as hydrates with (if defined) unknown stoichiometric composition.

### Preparation of Intermediate 1.1

### potassium (Z)-2-chloro-3-ethoxy-3-oxoprop-1-en-1-olate

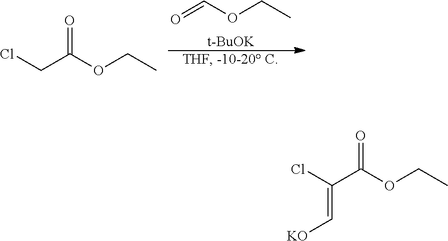

A solution of ethyl 2-chloroacetate (10 g, 81.60 mmol, 8.70 mL) and ethyl formate (6.04 g, 81.60 mmol, 6.56 mL) in tetrahydrofuran (THF) (150 mL) was stirred at −10° C. for 20 min, then t-BuOK (11.90 g, 106.08 mmol) was added in portions so that the temperature of the mixture remained below 0-5° C. The reaction was warmed to 20° C. for 16 hours. The reaction mixture was filtered to give a solid which was triturated with EtOAc (50 mL) for 5 hours at 20° C., filtered and the solid was dried under vacuum to give potassium (Z)-2-chloro-3-ethoxy-3-oxoprop-1-en-1-olate (12 g, 63.61 mmol, 77.95% yield) as a yellow solid.

¹H NMR (400 MHz, DMSO-d₆) δ 8.88-8.24 (m, 1H), 4.16 (q, J=7.2 Hz, 2H), 1.24 (t, J=7.2 Hz, 3H).

### Preparation of Intermediate 1.2

### ethyl 6-bromo-8-chloroimidazo[1,2-a]pyridine-3-carboxylate

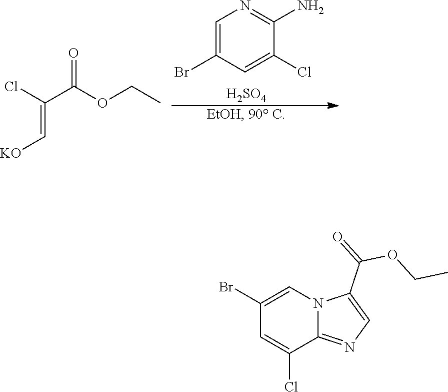

To a solution of 5-bromo-3-chloro-pyridin-2-amine (2 g, 9.64 mmol) and potassium (Z)-2-chloro-3-ethoxy-3-oxoprop-1-en-1-olate (7.27 g, 38.56 mmol) in EtOH (100 mL) at 20° C. was added H₂SO₄ (2.84 g, 28.92 mmol, 1.54 mL). The reaction mixture was heated to 90° C. for 16 hours. The reaction mixture was cooled to 20° C. EtOH was removed under reduced pressure, water (50 mL) was added and the mixture was extracted with EtAOc (3×80 mL). The combined organic layer was washed with brine (50 mL), dried over with Na₂SO₄, filtered and concentrated to give a residue, which was purified by flash silica gel chromatography (ISCO®; 40 g SepaFlash® Silica Flash Column, Eluent of 0˜30% Ethyl acetate/Petroleum ether gradient @80 mL/min) to give the product ethyl 6-bromo-8-chloroimidazo[1,2-a]pyridine-3-carboxylate (1.7 g, 5.21 mmol, 54.03% yield) as a white solid.

RT 0.888 min (method 1); m/z 304.9 (M+H)⁺ (ESI⁺); ¹H NMR (DMSO-d₆, 400 MHz): δ 9.24 (s, 1H), 8.301 (s, 1H), 8.05-8.04 (m, 1H), 4.40-4.35 (m, 2H), 1.35 (t, J=7.2 Hz, 3H).

### Preparation of Intermediate 1.3

### 6-bromo-8-chloroimidazo[1,2-a]pyridine-3-carbohydrazide

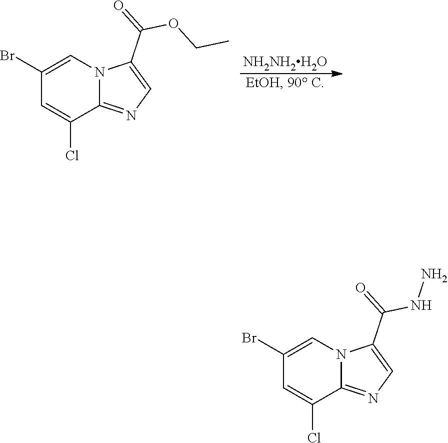

To a solution of ethyl 6-bromo-8-chloroimidazo[1,2-a]pyridine-3-carboxylate (1.7 g, 5.21 mmol, 93% purity) in EtOH (20 mL) at 20° C. was added NH₂NH₂·H₂O (3.26 g, 63.76 mmol, 3.16 mL, 98% purity). The mixture was refluxed for 2 h. and then cooled to 20° C. The precipitated solid was separated off to give the product 6-bromo-8-chloroimidazo[1,2-a]pyridine-3-carbohydrazide (1.5 g, 4.97 mmol, 95.49% yield) as a white solid.

RT 0.487 min (method 1); m/z 290.1 (M+H)⁺ (ESI⁺); ¹H NMR (DMSO-d₆, 400 MHz): 10.68-9.40 (m, 2H), 8.32 (s, 1H), 7.92 (d, J=1.6 Hz, 1H), 4.83-4.27 (m, 2H).

### Preparation of Intermediate 1.4

### 6-bromo-8-chloro-N′-(2,2-difluoroacetyl)imidazo[1,2-a]pyridine-3-carbohydrazide

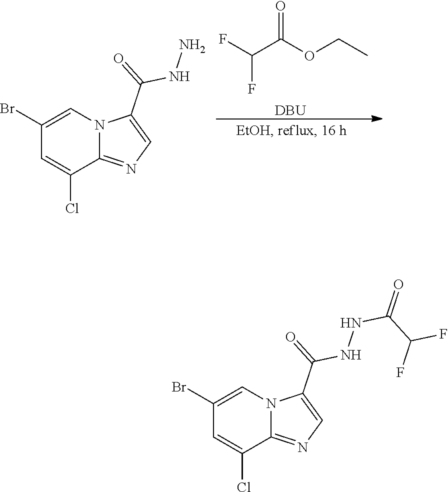

To a mixture of 6-bromo-8-chloroimidazo[1,2-a]pyridine-3-carbohydrazide (1.3 g, 4.31 mmol) and ethyl 2,2-difluoroacetate (5.35 g, 43.11 mmol) in EtOH (110 mL) at 20° C. was added 1,8-diazabicyclo[5.4.0]undec-7-ene (DBU) (1.31 g, 8.62 mmol). The mixture was refluxed for 16 hours before it was cooled to 20° C. and finally concentrated to give a residue, which was purified by flash silica gel chromatography (ISCO®; 20 g SepaFlash® Silica Flash Column, Eluent of 90% Ethyl acetate/Petroleum ether gradient @80 mL/min) to give the product 6-bromo-8-chloro-N′-(2,2-difluoroacetyl) imidazo[1,2-a]pyridine-3-carbohydrazide (0.85 g, 2.17 mmol) as a white solid.

RT 0.770 min (method 1); m/z 368.9 (M+H)⁺ (ESI⁺); ¹H NMR (DMSO-d₆, 400 MHz): 11.03-10.93 (m, 2H), 9.49 (s, 1H), 8.48 (s, 1H), 8.03 (s, 1H), 6.48 (t, J=52.8 Hz, 1H).

### Preparation of Intermediate 1.5

### 2-(6-bromo-8-chloroimidazo[1,2-a]pyridin-3-yl)-5-(difluoromethyl)-1,3,4-thiadiazole

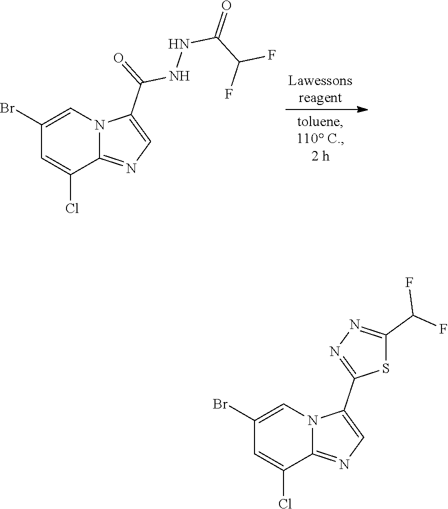 [SMILES: O=C(NNC(=O)C(F)F)c1cnc2c(Cl)cc(Br)cn12 ; FC(F)c1nnc(-c2cnc3c(Cl)cc(Br)cn23)s1]

To a solution of 6-bromo-8-chloro-N′-(2,2-difluoroacetyl)imidazo[1,2-a]pyridine-3-carbohydrazide (200.00 mg, 511.52 μmol) in toluene (4 mL)) at 20° C. was added Lawesson's reagent (227.58 mg, 562.67 μmol. The mixture was stirred at 110° C. for 2 hours. The mixture was cooled to 20° C. and concentrated to give a residue, which was triturated with MeOH (5 mL) for 30 min. after filtration, the cake was collected to give the product 2-(6-bromo-8-chloro-imidazo[1,2-a]pyridin-3-yl)-5-(difluoromethyl)-1,3,4-thiadiazole (140 mg, 382.95 μmol, 74.87% yield) as white solid.

¹H NMR (DMSO-d₆, 400 MHz): 7.54-7.84 (t, J=53.2 Hz, 1H), 8.08 (d, J=1.6 Hz, 1H), 8.64 (s, 1H), 9.61 (d, J=1.6 Hz, 1H).

### Preparation of Intermediate 1.6

### 2-(6-(benzylthio)-8-chloroimidazo[1,2-a]pyridin-3-yl)-5-(difluoromethyl)-1,3,4-thiadiazole

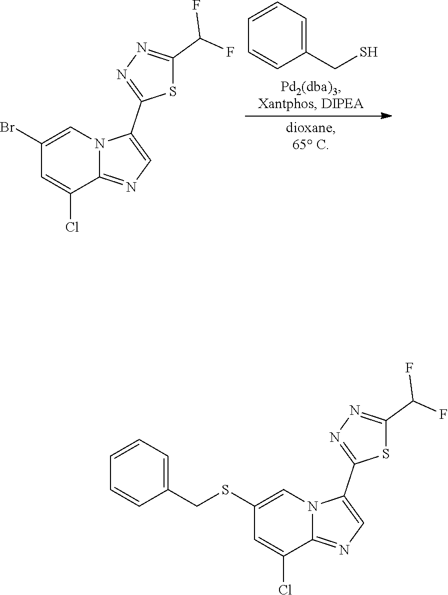

A mixture of 2-(6-bromo-8-chloro-imidazo[1,2-a]pyridin-3-yl)-5-(difluoromethyl)-1,3,4-thiadiazole (100 mg, 273.53 μmol), Xantphos (31.65 mg, 54.71 μmol), N,N-diisopropylethylamine (DIPEA) (70.70 mg, 547.07 μmol) and Pd₂(dba)₃ (tris(dinezylideneacetone)dipalladium(0)) (25.05 mg, 27.35 μmol) in dioxane (2 mL) was stirred at 20° C. N₂ was bubbled through the mixture for 5 min, and finally phenylmethanethiol (33.97 mg, 273.53 μmol, 32.05 μL) was added. The mixture was heated to 65° C. and stirred for 16 h. The reaction mixture was cooled to 20° C. and concentrated to give a residue, which was purified by flash silica gel chromatography (ISCO®; 20 g SepaFlash® Silica Flash Column, Eluent of 0˜30% Ethyl acetate/Petroleum ether gradient @30 mL/min) to give the product 2-(6-benzylsulfanyl-8-chloroimidazo[1,2-a]pyridin-3-yl)-5-(difluoromethyl)-1,3,4-thiadiazole (80 mg, 193.70 μmol, 70.81% yield) as a yellow solid.

RT 1.012 min (method 1); m/z 408.9 (M+H)⁺ (ESI⁺).

### Preparation of Intermediate 1.7

### 8-chloro-3-(5-(difluoromethyl)-1,3,4-thiadiazol-2-yl)imidazo[1,2-a]pyridine-6-sulfonyl chloride

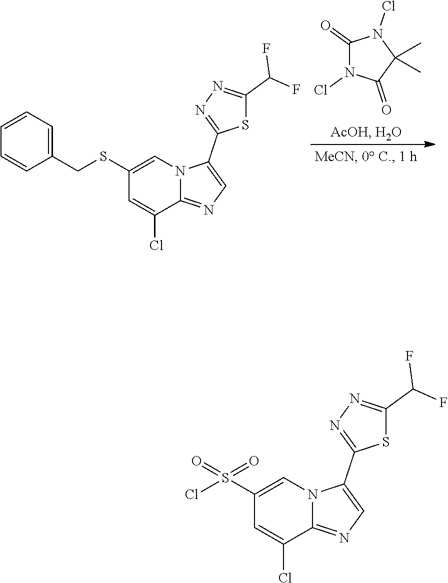

To a mixture of 2-(6-benzylsulfanyl-8-chloroimidazo[1,2-a]pyridin-3-yl)-5-(difluoromethyl)-1,3,4-thiadiazole (20 mg, 48.91 μmol), AcOH (5.29 mg, 88.05 μmol, 5.04 μL) and H₂O (881.21 μg, 48.91 μmol) in MeCN (0.5 mL) at 0° C. was added 1,3-dichloro-5,5-dimethyl-imidazolidine-2,4-dione (17.35 mg, 88.05 μmol). The mixture was stirred at 0° C. for 0.5 h. THE (3 mL) was added and the solution was dried over Na₂SO₄, filtered and concentrated to give the product 8-chloro-3-[5-(difluoromethyl)-1,3,4-thiadiazol-2-yl]imidazo [1,2-a]pyridine-6-sulfonyl chloride (18 mg, 46.73 μmol, 95.53% yield) as a white solid, which was used in the next step without further purification.

### Preparation of Example 1

### 8-chloro-N-(1-cyanocyclopropyl)-3-(5-(difluoromethyl)-1,3,4-thiadiazol-2-yl)imidazo[1,2-a]pyridine-6-sulfonamide

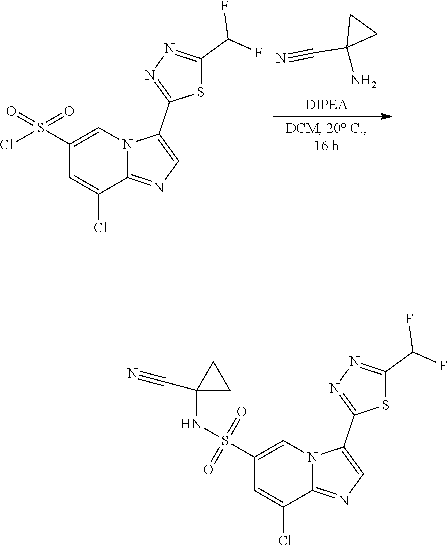

To a solution of 1-aminocyclopropane-1-carbonitrile (5.79 mg, 48.81 μmol, HCl) in pyridine (294.00 mg, 3.72 mmol) at 0° C. was added a solution of 8-chloro-3-(5-(difluoromethyl)-1,3,4-thiadiazol-2-yl)imidazo[1,2-a]pyridine-6-sulfonyl chloride (18.00 mg, 46.71 μmol) in THE (0.3 mL) dropwise over 1 min. The mixture was stirred at 20° C. for 16 hours. The reaction mixture was concentrated to give a residue, which was purified by preparative HPLC (column: Phenomenex C18 75*30 mm*3 μm; mobile phase: A: 0.225% formic acid in water, B: MeCN; B %: 30%-60%, 7 min) to give 8-chloro-N-(1-cyanocyclopropyl)-3-(5-(difluoromethyl)-1,3,4-thiadiazol-2-yl)imidazo[1,2-a]pyridine-6-sulfonamide (4.34 mg, 10.07 μmol, 20.64% yield) as a gray solid.

RT 0.882 min (method 1); m/z 430.9 (M+H)⁺ (ESI⁺); ¹H NMR (DMSO-d₆, 400 MHz): 9.95 (s, 1H), 8.76 (s, 1H), 8.25 (s, 1H), 7.87 (s, 1H), 7.71 (t, J=52.8 Hz, 1H), 1.23 (s, 2H), 1.15 (s, 2H).

### Preparation of Example 2

### 4-(6-(N-(1-cyanocyclopropyl)sulfamoyl)-3-(5-(difluoromethyl)-1,3,4-thiadiazol-2-yl)imidazo[1,2-a]pyridin-8-yl)-N,N-dimethylpiperazine-1-carboxamide

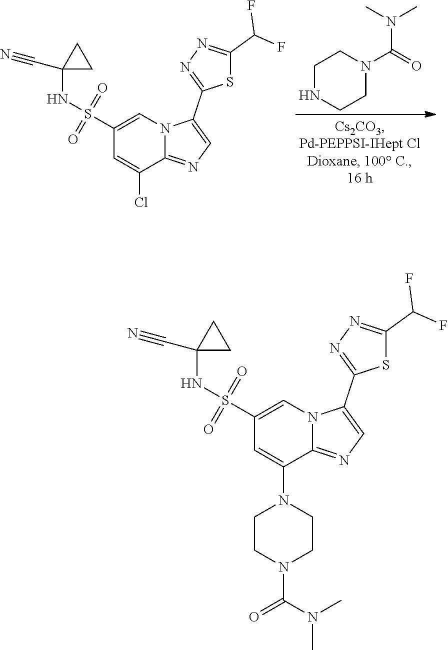

A mixture of 8-chloro-N-(1-cyanocyclopropyl)-3-(5-(difluoromethyl)-1,3,4-thiadiazol-2-yl)imidazo[1,2-a]pyridine-6-sulfonamide (30 mg, 69.63 μmol), N,N-dimethylpiperazine-1-carboxamide (13.14 mg, 83.56 μmol), Pd-PEPPSI-IHept Cl (6.77 mg, 6.96 μmol) and Cs₂CO₃ (68.06 mg, 208.89 μmol) in dioxane (0.5 mL) was degassed and purged with N₂ (3×) before it was stirred at 100° C. for 12 h under a N₂ atmosphere. The mixture was concentrated under vacuum and the residue was purified by preparative HPLC (column: Phenomenex Synergi C18 150*25 mm*10 μm; mobile phase: A: 0.225% formic acid in water, B: MeCN; B %: 30%-60%, 10 min) to give the product 4-(6-(N-(1-cyanocyclopropyl)sulfamoyl)-3-(5-(difluoromethyl)-1,3,4-thiadiazol-2-yl)imidazo[1,2-a]pyridin-8-yl)-N,N-dimethylpiperazine-1-carboxamide (15.36 mg, 39.7% yield, 99.29% purity) as an off-white solid.

RT 0.882 min (method 1); m/z 552.2 (M+H)⁺ (ESI⁺); ¹H NMR (DMSO-d₆, 400 MHz): 9.75 (s, 1H), 9.57 (br, 1H), 8.65 (s, 1H), 7.71 (t, J=53.2, 1H), 7.01 (s, 1H), 3.66-3.65 (m, 4H), 3.38-3.36 (m, 4H), 2.81 (s, 6H), 1.45-1.37 (m, 4H).

### Preparation of Example 3

### (R)—N-(1-cyanocyclopropyl)-3-(5-(difluoromethyl)-1,3,4-thiadiazol-2-yl)-8-(2-methylmorpholino)imidazo[1,2-a]pyridine-6-sulfonamide

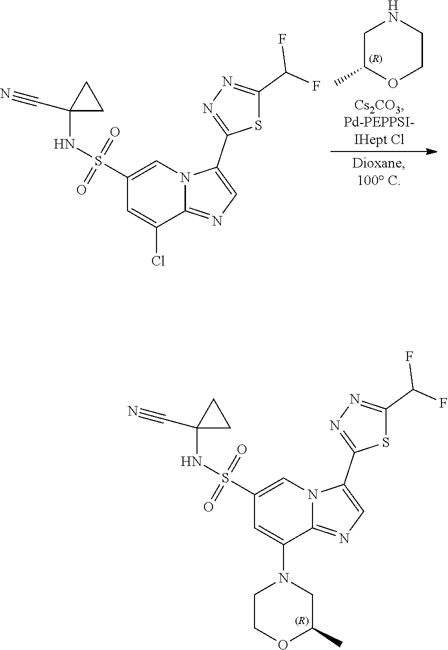

A mixture of 8-chloro-N-(1-cyanocyclopropyl)-3-[5-(difluoromethyl)-1,3,4-thiadiazol-2-yl]imidazo[1,2-a]pyridine-6-sulfonamide (10 mg, 23.21 μmol), (R)-2-methylmorpholine (2.82 mg, 27.85 μmol), Pd-PEPPSI-IHept Cl (2.26 mg, 2.32 μmol) and Cs₂CO₃ (22.69 mg, 69.63 μmol) in dioxane (0.5 mL) was degassed and purged with N₂ (3×) before it was stirred at 100° C. for 12 h under a N₂ atmosphere.

The mixture was filtered and concentrated under reduced pressure to give a residue, which was purified by preparative HPLC (column: Phenomenex Synergi C18 150*25 mm*10 μm; mobile phase: A: 0.225% formic acid in water, B: MeCN; B %: 32%-62%, 10 min) to afford the product (R)—N-(1-cyanocyclopropyl)-3-(5-(difluoromethyl)-1,3,4-thiadiazol-2-yl)-8-(2-methylmorpholino)imidazo[1,2-a]pyridine-6-sulfonamide (3.22 mg, 28.00% yield) as a yellow solid.

RT 0.870 (method 1); m/z 496.0 (M+H)⁺ (ES⁺); ¹H NMR (400 MHz, DMSO-d₆) δ 9.71 (d, J=1.2 Hz, 1H), 8.62 (s, 1H), 8.30 (s, 1H), 7.55-7.85 (t, J=53.2 Hz, 1H), 6.98 (d, J=1.2 Hz, 1H), 4.18-4.32 (m, 2H), 3.98 (dd, J=11.2, 1.6 Hz, 1H), 3.74-3.85 (m, 2H), 2.92 (m, 1H) 2.61-2.67 (m, 1H), 1.35-1.41 (m, 2H), 1.26-1.33 (m, 2H), 1.20 (d, J=6.4 Hz, 3H).

### Preparation of Intermediate 4.1

### (5-bromo-3-chloropyridin-2-yl)methanamine

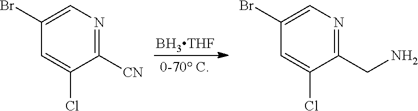 [SMILES: N#Cc1ncc(Br)cc1Cl ; NCc1ncc(Br)cc1Cl ; "BH3·THF"]

To a mixture of 5-bromo-3-chloropicolinonitrile (2.0 g, 9.20 mmol) in THE (10 mL) under ice-water cooling was added BH₃-THF (1 M, 11.04 mL) over 5 min. The mixture was stirred at 0° C. for 30 min before it was warmed to 20° C. and stirred for another 30 min at this temperature. The mixture was cooled to 0° C. and quenched with dropwise addition of MeOH (10 mL) over 5 min. The mixture was heated to 70° C. and stirred for 30 min at this temperature. The reaction was concentrated under vacuum to give the crude product (2.2 g) as a light brown solid. The crude product was dissolved in HCl (aq. 2M, 20 mL), washed with DCM (20 mL; 2×) and the aqueous phase was finally concentrated under vacuum to give the product (5-bromo-3-chloro-2-pyridyl)methanamine (1.5 g, 4.07 mmol, 44.26% yield, 70% purity, HCl salt) as alight brown solid.

RT 0.18 min (method 2); m/z 222.9 (M+H)⁺ (ESI⁺), ¹H NMR (400 MHz, DMSO-d₆) δ=8.78 (d, J=2.0 Hz, 1H), 8.69 (br, 3H), 8.47 (d, J=2.0 Hz, 1H), 4.24 (d, J=6.2 Hz, 2H).

### Preparation of Intermediate 4.2

### Ethyl 2-(((5-bromo-3-chloropyridin-2-yl)methyl)amino)-2-oxoacetate

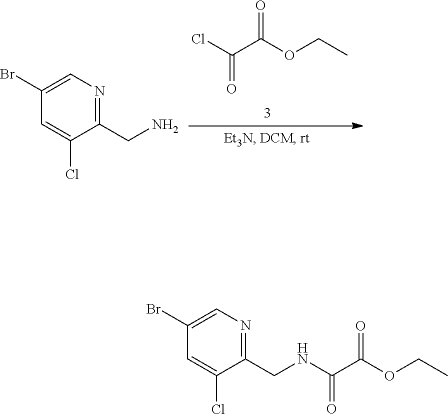

To a mixture of (5-bromo-3-chloro-2-pyridyl)methanamine (1.5 g, 5.82 mmol, HCl salt) in DCM (30 mL) under ice-water cooling was added DIPEA (2.25 g, 17.45 mmol). Then, ethyl 2-chloro-2-oxoacetate (952.77 mg, 6.98 mmol) was added over 5 min and the mixture was stirred at 0° C. for 30 min. The mixture was warmed to 20° C. and stirred for 30 min at this temperature. The mixture was quenched with aqueous NaHCO₃ solution (50 mL) and extracted with DCM (50 mL). The organic phase was separated, dried over Na₂SO₄, filtered and concentrated under vacuum. The residue was purified by column chromatography on silica gel (PE:EtOAc=10:1 to 1:1) to give the product ethyl 2-(((5-bromo-3-chloropyridin-2-yl)methyl)amino)-2-oxoacetate (1300 mg, 3.64 mmol, 62.57% yield, 65.6% purity) as a white solid. RT 0.61 min (method 1); m/z 322.8 (M+H)⁺ (ESI⁺). The product was used without further purification in the next step.

### Preparation of Intermediate 4.3

### Ethyl 6-bromo-8-chloroimidazo[1,5-a]pyridine-3-carboxylate

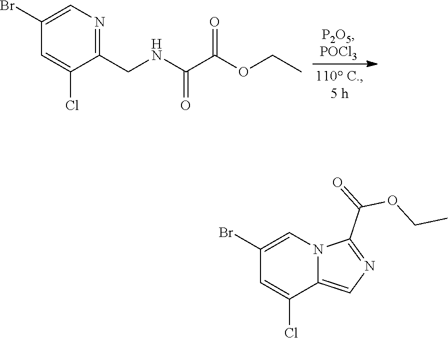 [SMILES: CCOC(=O)C(=O)NCc1ncc(Br)cc1Cl ; CCOC(=O)c1ncc2c(Cl)cc(Br)cn12]

To a mixture of ethyl ethyl 2-(((5-bromo-3-chloropyridin-2-yl)methyl)amino)-2-oxoacetate (1300 mg, 4.04 mmol) in POCl₃ (15 mL) under ice water cooling was added phosphorus pentoxide (2.87 g, 20.21 mmol). The mixture was heated to 110° C. and stirred for 5 h at this temperature. The mixture was cooled to 25° C. and concentrated under vacuum to give a residue. The residue was dissolved in EtOAc (50 mL) and washed with water (30 mL) and an aqueous NaHCO₃ solution (30 mL). Then it was finally concentrated under vacuum to give a residue. The residue was purified by column chromatography on silica gel (PE:EtOAc=10:1 to 3:1) to give the product ethyl 6-bromo-8-chloroimidazo[1,5-a]pyridine-3-carboxylate (900 mg, 2.97 mmol, 73.34% yield) as a white solid.

RT 0.718 min (method 1), m/z 304.8 (M+H)⁺ (ESI⁺), ¹H NMR (400 MHz, CHLOROFORM-d) 6=9.47 (s, 1H), 7.77 (s, 1H), 7.20 (s, 1H), 4.65-4.42 (m, 2H), 1.57-1.42 (m, 3H)

### Preparation of Intermediate 4.4

### 6-bromo-8-chloroimidazo[1,5-a]pyridine-3-carbohydrazide

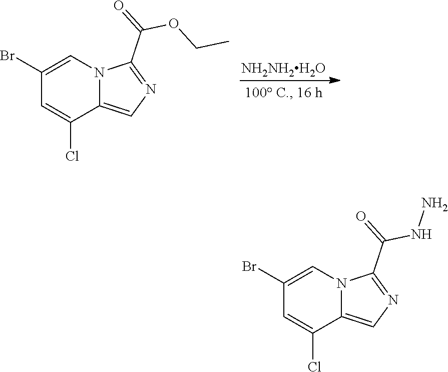

To a mixture of ethyl 6-bromo-8-chloroimidazo[1,5-a]pyridine-3-carboxylate (900 mg, 2.97 mmol) in EtOH (20 mL) was added NH₂NH₂—H₂O (1.48 g, 29.65 mmol, 98%). The mixture was heated to 80° C. and stirred for 2 h at this temperature. The reaction was cooled to 25° C. and the precipitated solid was separated off. The crude product was triturated with EtOH (5 mL) to give 6-bromo-8-chloroimidazo[1,5-a]pyridine-3-carbohydrazide (650 mg, 2.25 mmol, 75.72% yield) as a white solid.

RT 0.56 min (method 1); m/z 290.8 (M+H)⁺ (ESI⁺); ¹H NMR (400 MHz, DMSO-d₆) δ=10.02 (s, 1H), 9.50 (s, 1H), 7.72 (s, 1H), 7.51 (s, 1H), 4.58 (d, J=4.0 Hz, 2H).

### Preparation of Intermediate 4.5

### 6-bromo-8-chloro-N′-(2,2-difluoroacetyl)imidazo[1,5-a]pyridine-3-carbohydrazide

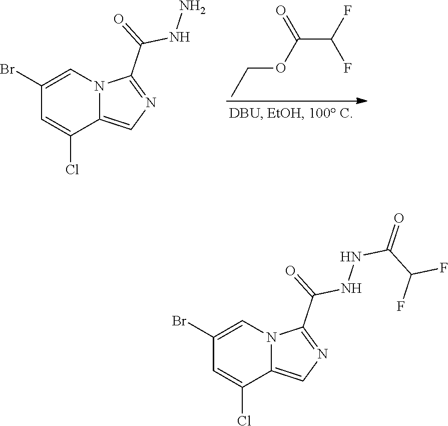

To a mixture of 6-bromo-8-chloroimidazo[1,5-a]pyridine-3-carbohydrazide (650 mg, 2.25 mmol) in EtOH (20 mL) was added ethyl 2,2-difluoroacetate (3.10 g, 22.45 mmol) and DBU (683.58 mg, 4.49 mmol). The mixture was heated to 100° C. stirred for 16 h at this temperature. The mixture was cooled to 25° C. and concentrated under vacuum. The residue was dissolved with DCM (50 mL), washed with an aqueous NH₄Cl solution (30 mL; 2×) and concentrated under vacuum to give the crude product. The crude product was purified by column chromatography on silica gel (PE/EtOAc=1:1 to MeOH:EtOAc=1:10) to give the product 6-bromo-8-chloro-N′-(2,2-difluoroacetyl)imidazo[1,5-a]pyridine-3-carbohydrazide (650 mg, 1.56 mmol, 69.32% yield, 88% purity) as a white solid.

RT 0.62 min (method 1); m/z 368.8 (M+H)⁺ (ESI⁺); ¹H NMR (400 MHz, DMSO-d₆) δ=10.95 (br, 2H), 9.44 (s, 1H), 7.81 (s, 1H), 7.59 (s, 1H), 6.38 (t, J=53.2, 1H).

### Preparation of Intermediate 4.6

### 2-(6-bromo-8-chloroimidazo[1,5-a]pyridin-3-yl)-5-(difluoromethyl)-1,3,4-thiadiazole

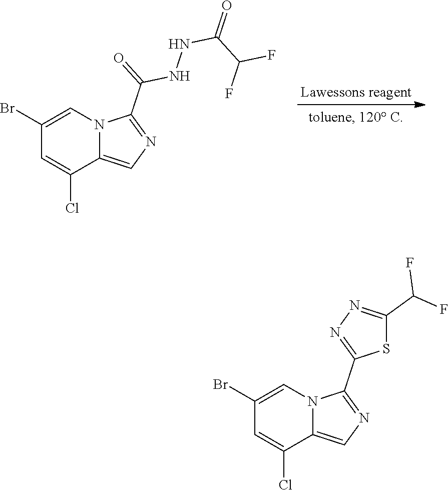 [SMILES: O=C(NNC(=O)C(F)F)c1ncc2c(Cl)cc(Br)cn12 ; FC(F)c1nnc(-c2ncc3c(Cl)cc(Br)cn23)s1]

To a mixture of 6-bromo-8-chloro-N′-(2,2-difluoroacetyl)imidazo[1,5-a]pyridine-3-carbohydrazide (550 mg, 1.50 mmol) in toluene (20 mL) was added Lawessons reagent (665.80 mg, 1.65 mmol) under a N₂ atmosphere. The reaction was heated to 120° C. and stirred for 2 h at this temperature. The reaction was cooled to 25° C. and concentrated under vacuum. The residue was triturated with MeOH (10 mL) at 70° C. for 1 h, filtered and the cake was collected, and dried under vacuum to give the product 2-(6-bromo-8-chloroimidazo[1,5-a]pyridin-3-yl)-5-(difluoromethyl)-1,3,4-thiadiazole (530 mg, 1.45 mmol, 96.88% yield) as a light yellow solid.

RT 0.806 min (method 1); m/z 366.8 (M+H)⁺ (ESI⁺); ¹H NMR (400 MHz, DMSO-d₆) δ=9.62 (s, 1H), 8.64 (s, 1H), 8.09 (s, 1H), 7.70 (t, J=53.2, 1H).

### Preparation of Intermediate 4.7

### 2-(6-(benzylthio)-8-chloroimidazo[1,5-a]pyridin-3-yl)-5-(difluoromethyl)-1,3,4-thiadiazole

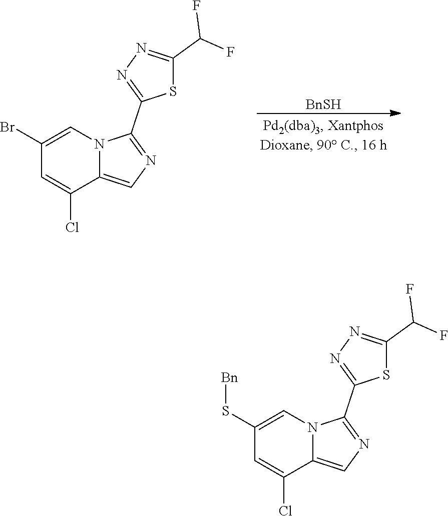

To a mixture of 2-(6-bromo-8-chloroimidazo[1,5-a]pyridin-3-yl)-5-(difluoromethyl)-1,3,4-thiadiazole (450 mg, 1.23 mmol) and phenylmethanethiol (168.17 mg, 1.35 mmol) in dioxane (10 mL) which was degassed with nitrogen for 2 min was added Pd₂(dba)₃ (112.72 mg, 123.09 μmol), Xantphos (71.22 mg, 123.09 μmol) and DIEA (477.26 mg, 3.69 mmol) under nitrogen. The mixture was heated to 90° C. and stirred for 16 h at this temperature. The mixture was filtered and concentrated under vacuum. The residue was purified by column chromatography on silica gel (PE:EtOAc=20:1 to 5:1) to give the product 2-(6-(benzylthio)-8-chloroimidazo[1,5-a]pyridin-3-yl)-5-(difluoromethyl)-1,3,4-thiadiazole (250 mg, 489.15 μmol, 39.74% yield, 80% purity) as a light yellow solid.

RT 0.99 min (method 1); m/z 409.0 (M+H)⁺ (ESI⁺); ¹H NMR (400 MHz, CHLOROFORM-d) 5=9.35 (s, 1H), 7.69-7.67 (m, 1H), 7.39-7.28 (m, 2H), 7.25-7.12 (m, 3H), 7.05 (t, J=53.2, 1H), 7.00 (s, 1H), 6.90 (s, 1H), 4.10 (s, 2H)

### Preparation of Intermediate 4.8

### 2-(6-(benzylthio)-8-chloro-1-iodoimidazo[1,5-a]pyridin-3-yl)-5-(difluoromethyl)-1,3,4-thiadiazole

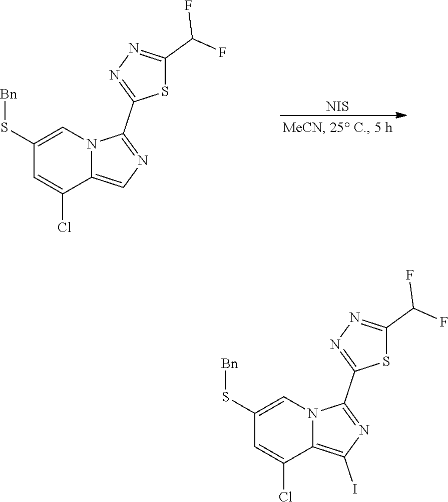 [SMILES: FC(F)c1nnc(-c2ncc3c(Cl)cc(SCc4ccccc4)cn23)s1 ; "NIS" ; FC(F)c1nnc(-c2nc(I)c3c(Cl)cc(SCc4ccccc4)cn23)s1]

To a mixture of 2-(6-(benzylthio)-8-chloroimidazo[1,5-a]pyridin-3-yl)-5-(difluoromethyl)-1,3,4-thiadiazole (130 mg, 317.95 μmol) in MeCN (5 mL) at 0° C. was added NIS (78.68 mg, 349.74 μmol). The mixture was stirred at 25° C. for 5 h. The reaction mixture was used for the next step directly.

RT 0.99 min (method 1); m/z 535.0 (M+H)⁺ (ESI⁺)

### Preparation of Intermediate 4.9

### 8-chloro-3-(5-(difluoromethyl)-1,3,4-thiadiazol-2-yl)-1-iodoimidazo[1,5-a]pyridine-6-sulfonyl chloride

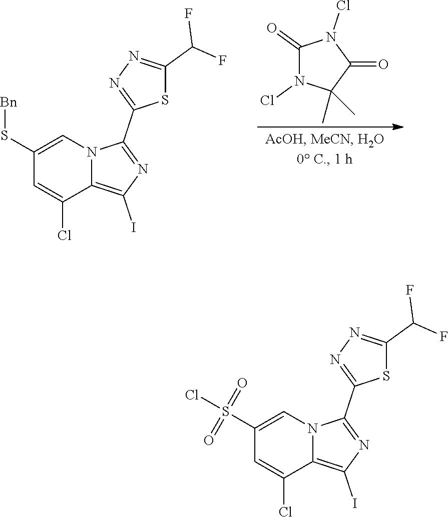 [SMILES: FC(F)c1nnc(-c2nc(I)c3c(Cl)cc(SCc4ccccc4)cn23)s1 ; CC1(C)C(=O)N(Cl)C(=O)N1Cl ; O=S(=O)(Cl)c1cc(Cl)c2c(I)nc(-c3nnc(C(F)F)s3)n2c1]

A mixture of 2-(6-(benzylthio)-8-chloro-1-iodoimidazo[1,5-a]pyridin-3-yl)-5-(difluoromethyl)-1,3,4-thiadiazole (170 mg, 317.95 μmol) in MeCN (5 mL) was cooled to 0° C. before H₂O (5.73 mg, 317.95 μmol), AcOH (38.19 mg, 635.89 μmol) and 1,3-dichloro-5,5-dimethylimidazolidine-2,4-dione (125.28 mg, 635.89 μmol) was added. The mixture was stirred at 0° C. for 2 h. The mixture was diluted with THE (8 mL), dried over Na₂SO₄, filtered and concentrated under vacuum to give the crude product 8-chloro-3-(5-(difluoromethyl)-1,3,4-thiadiazol-2-yl)-1-iodoimidazo[1,5-a]pyridine-6-sulfonyl chloride (160 mg, 219.14 μmol, 68.92% yield, 70% purity) as a light brown oil.

It is noted that it cannot be excluded that the dichloro-compound 1,8-dichloro-3-(5-(difluoromethyl)-1,3,4-thiadiazol-2-yl)imidazo[1,5-a]pyridine-6-sulfonyl chloride (see intermediate 18.2) was also formed in this process.

### Preparation of Intermediate 4.10

### 8-chloro-N-(1-cyanocyclopropyl)-3-(5-(difluoromethyl)-1,3,4-thiadiazol-2-yl)-1-iodoimidazo[1,5-a]pyridine-6-sulfonamide

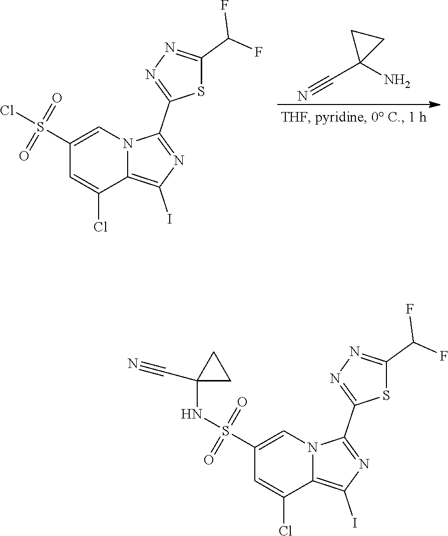 [SMILES: O=S(=O)(Cl)c1cc(Cl)c2c(I)nc(-c3nnc(C(F)F)s3)n2c1 ; N#CC1(N)CC1 ; N#CC1(NS(=O)(=O)c2cc(Cl)c3c(I)nc(-c4nnc(C(F)F)s4)n3c2)CC1]

To a mixture of 1-aminocyclopropane-1-carbonitrile (128.51 mg, 1.57 mmol, HCl salt) in pyridine (3 mL) at 0° C. was added 8-chloro-3-(5-(difluoromethyl)-1,3,4-thiadiazol-2-yl)-1-iodoimidazo[1,5-a]pyridine-6-sulfonyl chloride (160 mg, 313.05 μmol) in THF (2 mL). The mixture was stirred at 0° C. for 1 h. The reaction was concentrated under vacuum. The residue was dissolved in DCM (20 mL) and washed with brine (20 mL), dried over Na₂SO₄, filtered and concentrated under vacuum. The residue was purified by column chromatography on silica gel (PE:EtOAc=10:1 to 3:1) to give the product 8-chloro-N-(1-cyanocyclopropyl)-3-(5-(difluoromethyl)-1,3,4-thiadiazol-2-yl)-1-iodoimidazo[1,5-a]pyridine-6-sulfonamide (110 mg, 128.43 μmol, 41.02% yield, 65% purity) as a yellow solid.

RT 0.966 min (method 1); m/z 556.9 (M+H)⁺ (ESI⁺); ¹H NMR (400 MHz, DMSO-d₆) δ=10.00 (s, 1H), 9.70 (br, 1H), 7.72 (t, J=53.2, 1H), 7.48 (d, J=1.1 Hz, 1H), 1.54-1.48 (m, 2H), 1.42-1.35 (m, 2H)

### Preparation of Example 4

### 8-chloro-N-(1-cyanocyclopropyl)-3-(5-(difluoromethyl)-1,3,4-thiadiazol-2-yl)imidazo[1,5-a]pyridine-6-sulfonamide

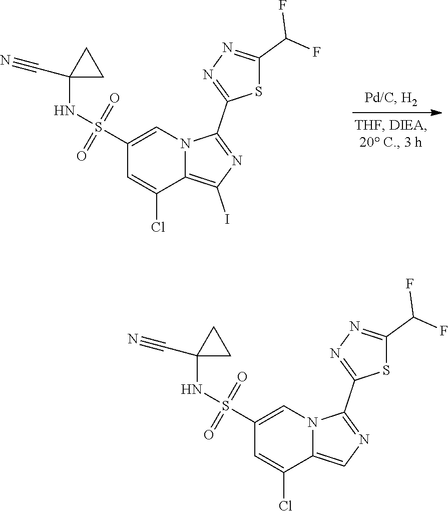 [SMILES: N#CC1(NS(=O)(=O)c2cc(Cl)c3c(I)nc(-c4nnc(C(F)F)s4)n3c2)CC1 ; N#CC1(NS(=O)(=O)c2cc(Cl)c3cnc(-c4nnc(C(F)F)s4)n3c2)CC1]

To a mixture of 8-chloro-N-(1-cyanocyclopropyl)-3-(5-(difluoromethyl)-1,3,4-thiadiazol-2-yl)-1-iodoimidazo[1,5-a]pyridine-6-sulfonamide (15 mg, 26.94 μmol) in THF (5 mL) at 20° C. was added DIPEA (17.41 mg, 134.71 μmol) and Pd/C (5 mg, 10% purity). The mixture was stirred under H₂ (15 psi) at 20° C. for 2 h. The mixture was filtered and concentrated under vacuum. The residue was purified by preparative HPLC (column: Unisil 3-100 C18 Ultra 150*50 mm*3 μm; mobile phase: A: 0.225% formic acid in water, B: MeCN; B %: 33%-63%, 15 min) to give 8-chloro-N-(1-cyanocyclopropyl)-3-(5-(difluoromethyl)-1,3,4-thiadiazol-2-yl)imidazo[1,5-a]pyridine-6-sulfonamide (4.02 mg, 9.23 μmol, 34.25% yield, 98.9% purity, formic acid salt) as a light yellow solid.

RT 0.966 min (method 1); m/z 431.1 (M+H)⁺ (ESI⁺); H NMR (400 MHz, METHANOL-d₄) δ=10.25 (s, 1H), 8.56 (br, 1H), 7.99 (s, 1H), 7.51 (s, 1H), 7.36 (t, J=53.2 Hz, 1H), 1.54-1.50 (m, 2H), 1.50-1.46 (m, 2H).

### Preparation of Example 5

### 4-(6-(N-(1-cyanocyclopropyl)sulfamoyl)-3-(5-(difluoromethyl)-1,3,4-thiadiazol-2-yl)imidazo[1,5-a]pyridin-8-yl)-N,N-dimethylpiperazine-1-carboxamide

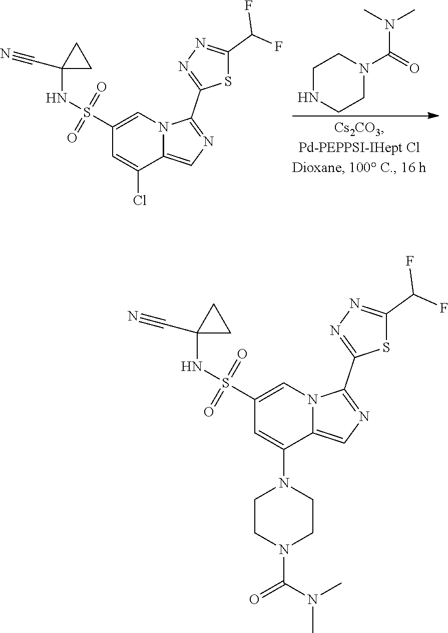

To a mixture of 8-chloro-N-(1-cyanocyclopropyl)-3-(5-(difluoromethyl)-1,3,4-thiadiazol-2-yl)imidazo[1,5-a]pyridine-6-sulfonamide (70 mg, 162.47 μmol) in 1,4-dioxane (1 mL) which was degassed with nitrogen for 2 min was added N,N-dimethylpiperazine-1-carboxamide (38.31 mg, 243.71 μmol), Cs₂CO₃ (105.87 mg, 324.95 μmol) and Pd-PEPPSI-IHept Cl (15.80 mg, 16.25 μmol). The mixture was stirred at 100° C. for 16 h. The mixture was concentrated under vacuum. The residue was purified by preparative HPLC (column: 3_Phenomenex Luna C18 75*30 mm*3 um; mobile phase: A: 0.1% TFA in water, B: MeCN; B %: 48%-68%, 7 min) to give 6 mg product with 80% purity. After that, the 80% purity product was further purified by preparative HPLC (column: Waters Xbridge 150*25 mm*5 μm; mobile phase: A: 1 mM aqueous solution of NH₄HCO₃, B: MeCN; B %: 35%-53%, 10 min) to give the product 4-(6-(N-(1-cyanocyclopropyl)sulfamoyl)-3-(5-(difluoromethyl)-1,3,4-thiadiazol-2-yl)imidazo[1,5-a]pyridin-8-yl)-N,N-dimethylpiperazine-1-carboxamide (2.48 mg, 4.39 μmol, 2.70% yield, 97.7% purity) as a light yellow solid.

RT 0.72 min (method 1); m/z 552.0 (M+H)⁺ (ESI⁺); ¹H NMR (400 MHz, METHANOL-d₄) δ=9.93 (s, 1H), 7.93 (s, 1H), 7.35 (t, J=53.2, 1H), 6.79 (s, 1H), 3.61-3.52 (m, 4H), 3.44-3.41 (m, 4H), 2.92 (s, 6H), 1.57-1.50 (m, 2H), 1.49-1.42 (m, 2H).

### Preparation of Intermediate 6.1

### 1,8-dichloro-3-(5-(difluoromethyl)-1,3,4-thiadiazol-2-yl)imidazo[1,5-a]pyridine-6-sulfonyl chloride

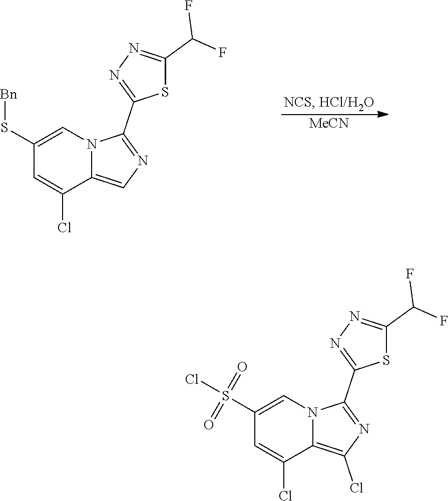 [SMILES: "MeCN" ; FC(F)c1nnc(-c2ncc3c(Cl)cc(SCc4ccccc4)cn23)s1 ; "NCS, HCl/H2O" ; O=S(=O)(Cl)c1cc(Cl)c2c(Cl)nc(-c3nnc(C(F)F)s3)n2c1]

To a mixture of NCS (65.32 mg, 489.15 μmol, 4 eq) in MeCN (2 mL) at 0° C. was added an aqueous solution of HCl (2M, 244.57 μL) and 2-(6-benzylsulfanyl-8-chloroimidazo[1,5-a]pyridin-3-yl)-5-(difluoromethyl)-1,3,4-thiadiazole (50 mg, 122.29 μmol). The mixture was warmed to 20° C. and stirred for 1 h at this temperature. The mixture was diluted with DCM (10 mL) and washed with water (10 mL), dried over Na₂SO₄, filtered and concentrated under vacuum to give the crude product 1,8-dichloro-3-(5-(difluoromethyl)-1,3,4-thiadiazol-2-yl)imidazo[1,5-a]pyridine-6-sulfonyl chloride (50 mg, 94.13 μmol, 76.97% yield, 79% purity) as a yellow oil. The crude product was directly used in the next step without further purification.

### Preparation of Example 6

### 1,8-dichloro-N-(1-cyanocyclopropyl)-3-(5-(difluoromethyl)-1,3,4-thiadiazol-2-yl)imidazo[1,5-a]pyridine-6-sulfonamide

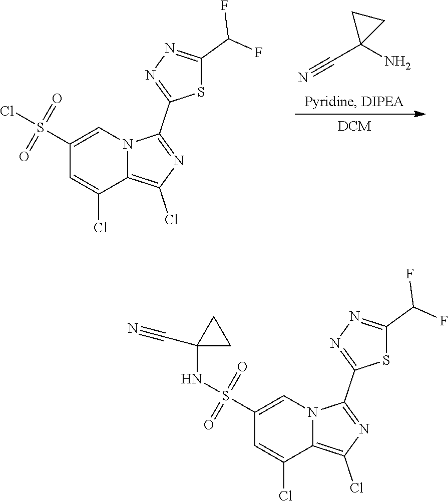 [SMILES: O=S(=O)(Cl)c1cc(Cl)c2c(Cl)nc(-c3nnc(C(F)F)s3)n2c1 ; N#CC1(N)CC1 ; N#CC1(NS(=O)(=O)c2cc(Cl)c3c(Cl)nc(-c4nnc(C(F)F)s4)n3c2)CC1]

To a mixture of 1,8-dichloro-3-(5-(difluoromethyl)-1,3,4-thiadiazol-2-yl)imidazo[1,5-a]pyridine-6-sulfonyl chloride (50 mg, 94.13 μmol, 79% purity) and DIPEA (24.33 mg, 188.26 μmol) in DCM (2 mL) was added 1-aminocyclopropane-1-carbonitrile (15.46 mg, 188.26 μmol, HCl salt). The mixture was stirred for 1 h at 20° C. Pyridine (148.91 mg, 1.88 mmol, 151.95 μL) was added and the mixture was stirred at 20° C. for another 16 h. The mixture was concentrated to give a residue, which was purified by preparative HPLC (column: 3_Phenomenex Luna C18 75*30 mm*3 um; mobile phase: A: 0.1% TFA in water, B: MeCN; B %: 50%-80%, 7 min) to give 1,8-dichloro-N-(1-cyanocyclopropyl)-3-(5-(difluoromethyl)-1,3,4-thiadiazol-2-yl)imidazo[1,5-a]pyridine-6-sulfonamide (10 mg, 21.49 μmol, 66.67% yield) as a yellow solid.

RT 0.87 min (method 1); m/z 464.9 (M+H)⁺ (ESI⁺); ¹H NMR (400 MHz, DMSO-d₆) δ=9.91 (s, 1H), 9.71 (s, 1H), 7.71 (t, J=53.2, 1H), 7.50 (s, 1H), 1.60-1.45 (m, 2H), 1.42-1.30 (m, 2H).

### Preparation of Example 7

### N-(1-cyanocyclopropyl)-3-(5-(difluoromethyl)-1,3,4-thiadiazol-2-yl)-8-(2-oxa-7-azaspiro[3.5]nonan-7-yl)imidazo[1,2-a]pyridine-6-sulfonamide

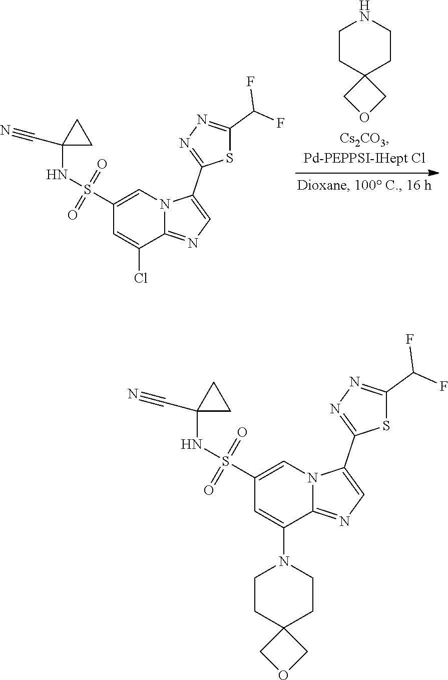 [SMILES: N#CC1(NS(=O)(=O)c2cc(Cl)c3ncc(-c4nnc(C(F)F)s4)n3c2)CC1 ; C1CC2(CCN1)COC2 ; N#CC1(NS(=O)(=O)c2cc(N3CCC4(CC3)COC4)c3ncc(-c4nnc(C(F)F)s4)n3c2)CC1]

A mixture of 8-chloro-N-(1-cyanocyclopropyl)-3-(5-(difluoromethyl)-1,3,4-thiadiazol-2-yl)imidazo[1,2-a]pyridine-6-sulfonamide (10.0 mg, 23.21 μmol), 2-oxa-7-azaspiro[3.5]nonane (4.4 mg, 34.82 μmol), Pd-PEPPSI-IHept Cl (2.3 mg, 2.32 μmol) and Cs₂CO₃ (22.7 mg, 69.63 μmol) in dioxane (0.5 mL) was degassed and purged with nitrogen (3×) and then stirred at 100° C. for 12 h under a nitrogen atmosphere. The mixture was filtered and the filtrate was concentrated under reduced pressure to give a residue, which was purified by preparative HPLC (column: Unisil 3-100 C18 Ultra 150*50 mm*3 μm; mobile phase: A: 0.225% formic acid in water, B: MeCN; B %: 33%-53%, 10 min) to give the product N-(1-cyanocyclopropyl)-3-(5-(difluoromethyl)-1,3,4-thiadiazol-2-yl)-8-(2-oxa-7-azaspiro[3.5]nonan-7-yl)imidazo[1,2-a]pyridine-6-sulfonamide (4.3 mg, 8.24 μmol) as a yellow solid.

RT 0.877 (method 1); m/z 522.1 (M+H)⁺ (ES⁺); ¹H NMR (400 MHz, DMSO-d₆) δ9.62 (s, 1H), 8.58 (s, 1H), 8.37 (s, 1H), 7.69 (t, J=53.2 Hz, 1H), 6.97 (s, 1H), 4.39 (s, 4H), 3.55-3.53 (m, 4H), 1.97-2.01 (m, 4H), 1.28-1.24 (m, 2H), 1.22-1.18 (m, 2H).

### Preparation of Example 8

### N-(1-cyanocyclopropyl)-3-(5-(difluoromethyl)-1,3,4-thiadiazol-2-yl)-8-(4-isobutyrylpiperazin-1-yl)imidazo[1,2-a]pyridine-6-sulfonamide

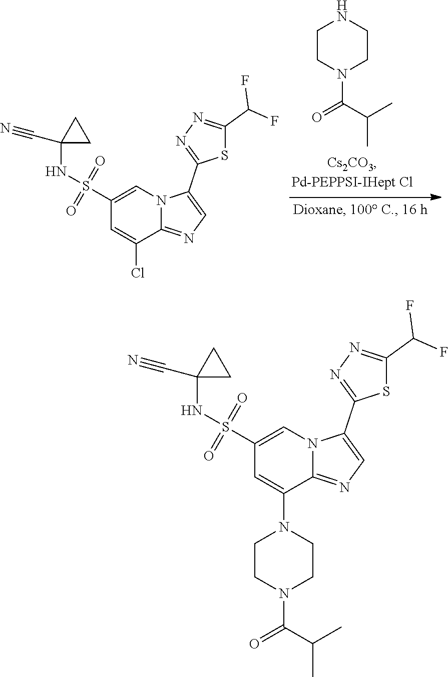

A mixture of 8-chloro-N-(1-cyanocyclopropyl)-3-(5-(difluoromethyl)-1,3,4-thiadiazol-2-yl)imidazo[1,2-a]pyridine-6-sulfonamide (10.0 mg, 23.21 μmol), 2-methyl-1-(piperazin-1-yl)propan-1-one (4.4 mg, 27.85 μmol), Pd-PEPPSI-IHept Cl (2.3 mg, 2.32 μmol) and Cs₂CO₃ (22.7 mg, 69.63 μmol) in dioxane (0.5 mL) was degassed and purged with nitrogen (3×) and then stirred at 100° C. for 12 h under a nitrogen atmosphere. The mixture was filtered, and the filtrate was concentrated under reduced pressure to give a residue, which was purified by preparative HPLC (column: Phenomenex C18 75*30 mm*3 um; mobile phase: A: 0.225% formic acid in water, B: MeCN; B %: 35%-65%, 7 min) to give the product N-(1-cyanocyclopropyl)-3-(5-(difluoromethyl)-1,3,4-thiadiazol-2-yl)-8-(4-isobutyrylpiperazin-1-yl)imidazo[1,2-a]pyridine-6-sulfonamide (2.3 mg, 4.16 μmol) as a light yellow solid.

RT 0.867 min (method 1); m/z 551.0 (M+H)⁺ (ESI⁺); ¹H NMR (400 MHz, MeOH-d₄) δ=9.67 (s, 1H), 8.62 (s, 1H), 8.40 (s, 1H), 7.53-7.91 (t, J=53.2 Hz, 1H), 6.99 (d, J=1.2 Hz, 1H), 3.76 (d, J=4.8 Hz, 2H), 3.71 (s, 2H), 3.63 (s, 2H), 3.56 (s, 2H), 2.92-2.99 (m, 1H), 1.23-1.31 (m, 2H), 1.17-1.23 (m, 2H), 1.05 (d, J=6.4 Hz, 6H).

### Preparation of Intermediate 9.1

### tert-butyl methyl(2-(N-methylisobutyramido)ethyl)carbamate

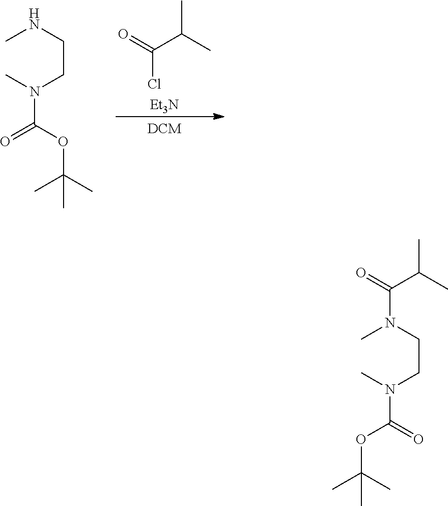

A mixture of tert-butyl methyl(2-(methylamino)ethyl)carbamate (300.0 mg, 1.59 mmol) and Et₃N (322.5 mg, 3.19 mmol) in DCM (3 mL) was degassed and purged with nitrogen (3×), then 2-methylpropanoyl chloride (186.8 mg, 1.75 mmol) was added dropwise at 0° C. The mixture was stirred at 20° C. for 1 h under a nitrogen atmosphere before it was poured into a saturated aqueous NaHCO₃ solution (10 mL) and extracted with DCM (10 mL, 3×). The combined organic layer was washed with brine (25 mL, 2×), dried over Na₂SO₄, filtered and concentrated to afford the product tert-butyl methyl (2-(N-methylisobutyramido)ethyl)carbamate (300.0 mg, 1.16 mmol) as a yellow oil.

¹H NMR (400 MHz, DMSO-d₆) δ 3.36-3.45 (m, 2H), 3.28 (t, J=6.4 Hz, 3H) 2.99 (br s, 1H) 2.74-2.83 (m, 5H) 1.38 (s, 9H), 0.93-1.00 (m, 6H).

### Preparation of Intermediate 9.2

### N-methyl-N-(2-(methylamino)ethyl)isobutyramide

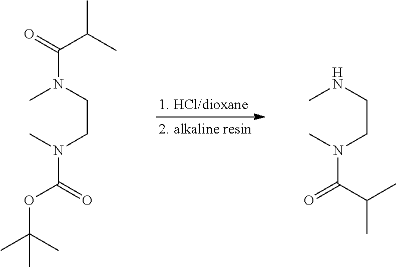 [SMILES: CC(C)C(=O)N(C)CCN(C)C(=O)OC(C)(C)C ; CNCCN(C)C(=O)C(C)C]

To a solution of tert-butyl methyl(2-(N-methylisobutyramido)ethyl)carbamate (300.0 mg, 1.16 mmol) in DCM (3 mL) was added a solution of HCl in dioxane (4 M, 3 mL). The mixture was stirred at 20° C. for 2 h. The mixture was concentrated under reduced pressure to give a residue. The residue was dissolved in MeOH (2 mL) and alkaline resin (2 g) was added. The mixture was stirred at 20° C. for 0.5 h. The mixture was filtered and concentrated to afford the product N-methyl-N-(2-(methylamino)ethyl)isobutyramide (116 mg, 0.73 mmol) as a colorless oil.

¹H NMR (400 MHz, CDCl₃) δ 3.70 (t, J=6.0 Hz, 2H) 3.15 (s, 3H) 3.11 (t, J=6.0 Hz, 2H) 2.79-2.87 (m, 1H) 2.69 (s, 3H) 1.12 (d, J=6.8 Hz, 6H).

### Preparation of Example 9

### N-(2-((6-(N-(1-cyanocyclopropyl)sulfamoyl)-3-(5-(difluoromethyl)-1,3,4-thiadiazol-2-yl)imidazo[1,2-a]pyridin-8-yl)(methyl)amino)ethyl)-N-methylisobutyramide

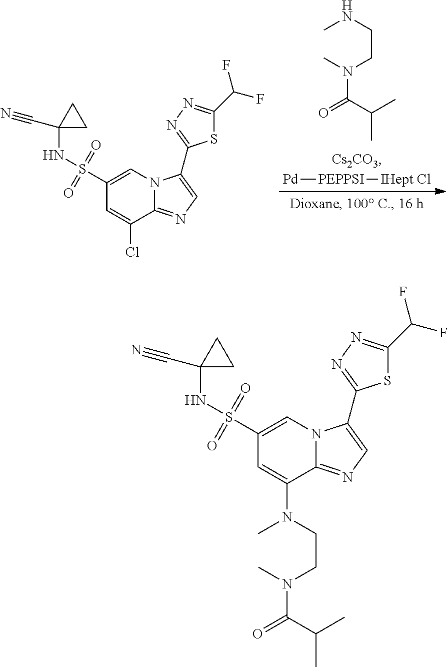

A mixture of 8-chloro-N-(1-cyanocyclopropyl)-3-(5-(difluoromethyl)-1,3,4-thiadiazol-2-yl)imidazo[1,2-a]pyridine-6-sulfonamide (10.0 mg, 23.21 μmol), N-methyl-N-(2-(methylamino)ethyl)isobutyramide (4.4 mg, 27.85 μmol), Pd-PEPPSI-IHept Cl (2.3 mg, 2.32 μmol) and Cs₂CO₃ (22.7 mg, 69.63 μmol) in dioxane (0.5 mL) was degassed and purged with nitrogen (3×) and stirred at 100° C. for 12 h under a nitrogen atmosphere. The mixture was concentrated under reduced pressure to give a residue which was purified by preparative HPLC (column: Phenomenex C18 75*30 mm*3 μm; mobile phase: A: 0.225% formic acid in water, B: MeCN; B %: 38%-68%, 7 min) to afford the product N-(2-((6-(N-(1-cyanocyclopropyl)sulfamoyl)-3-(5-(difluoromethyl)-1,3,4-thiadiazol-2-yl)imidazo[1,2-a]pyridin-8-yl)(methyl)amino)ethyl)-N-methylisobutyramide (1.4 mg, 2.53 μmol) as a yellow solid.

RT 0.898 min (method 1); m/z 553.0 (M+H)⁺ (ESI⁺); ¹H NMR (400 MHz, CDCl₃) 59.85 (d, J=1.6 Hz, 1H), 8.13 (s, 1H), 7.76 (s, 1H), 7.40 (d, J=1.2 Hz, 1H), 7.10 (t, J=53.2 Hz, 1H), 3.68-3.78 (m, 4H), 3.59 (s, 3H), 3.14 (s, 3H), 2.83 (m, 1H), 1.63 (s, 2H), 1.38-1.42 (m, 2H), 1.12-1.16 (m, 6H).

### Preparation of Intermediate 10.1

### 5-chloro-6-vinylpyridin-3-amine

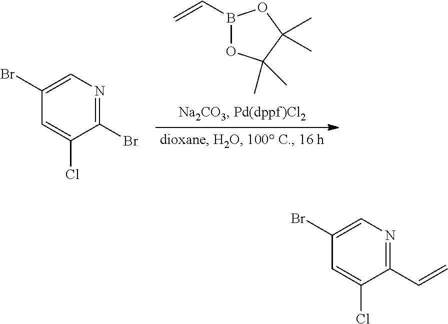

To a mixture of 2,5-dibromo-3-chloropyridine (1.60 g, 5.90 mmol), 4,4,5,5-tetramethyl-2-vinyl-1,3,2-dioxaborolane (1.36 g, 8.85 mmol) and Na₂CO₃ (1.25 g, 11.79 mmol) in dioxane (15 mL) and water (1 mL) was added Pd(dppf)Cl₂ (431.5 mg, 589.67 μmol) under a nitrogen atmosphere. The mixture was stirred at 100° C. for 16 h. The mixture was concentrated in vacuum to give a residue, which was purified by flash silica gel chromatography (ISCO®; 20 g SepaFlash® Silica Flash Column, eluent 0-20% DCM/PE gradient @35 mL/min) to give the product 5-bromo-3-chloro-2-vinylpyridine (1.80 g, 8.24 mmol; resulting from 3 reactions conducted in parallel) as a yellow oil.

¹H NMR (400 MHz, CDCl₃) δ 8.53 (d, J=2.0 Hz, 1H), 7.84 (d, J=2.0 Hz, 1H), 7.17 (dd, J=16.8, 10.8 Hz, 1H), 6.50 (dd, J=17.2, 2.0 Hz, 1H), 5.63 (dd, J=10.4, 1.6 Hz, 1H).

### Preparation of Intermediate 10.2

### methyl 6-bromo-8-chloroindolizine-3-carboxylate

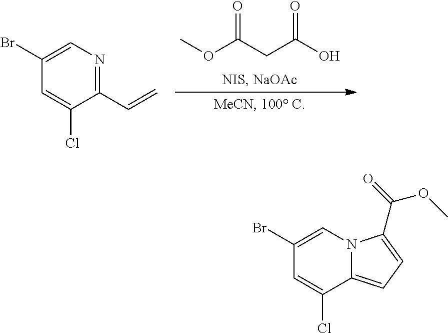

To a solution of 5-bromo-3-chloro-2-vinylpyridine (2.20 g, 10.07 mmol) and 3-methoxy-3-oxopropanoic acid (2.38 g, 20.14 mmol) in MeCN (40 mL) was added NIS (7.93 g, 35.24 mmol) and NaOAc (2.48 g, 30.21 mmol). The mixture was stirred at 100° C. for 16 h under a nitrogen atmosphere. The mixture was concentrated in vacuum to give a residue, which was purified by flash silica gel chromatography (ISCO®; 20 g SepaFlash® Silica Flash Column, eluent 0-10% EtOAc/PE gradient @35 mL/min) to give the product methyl 6-bromo-8-chloroindolizine-3-carboxylate (1.76 g, 6.10 mmol) as a yellow oil.

RT 1.053 min (method 1); m/z 289.9 (M+H)⁺ (ESI⁺); ¹H NMR (400 MHz, CDCl₃) δ 9.48 (s, 1H), 7.42 (d, J=4.8 Hz, 1H), 7.10 (d, J=1.6 Hz, 1H), 6.63 (d, J=4.4 Hz, 1H), 3.85 (s, 3H).

### Preparation of Intermediate 10.3

### 6-bromo-8-chloroindolizine-3-carbohydrazide

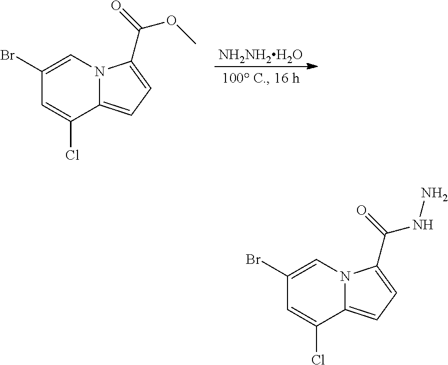

To a solution of methyl 6-bromo-8-chloroindolizine-3-carboxylate (1.76 g, 6.10 mmol) in EtOH (20 mL) was added N₂H₄—H₂O (3.74 g, 73.20 mmol, 98% purity). The mixture was heated to 100° C. and stirred for 2 h. The mixture was cool down to 25° C. and filtered. The cake was dried in vacuum to give the product 6-bromo-8-chloroindolizine-3-carbohydrazide (1.36 g, 4.71 mmol) as a white solid.

RT 0.808 min (method 1); m/z 289.9 (M+H)⁺ (ESI⁺); ¹H NMR (400 MHz, DMSO-d₆) δ 9.69-9.78 (m, 2H), 7.90 (br, 2H), 7.60 (d, J=4.4 Hz, 1H), 7.41 (d, J=1.6 Hz, 1H), 6.72 (dd, J=4.4, 0.4 Hz, 1H).

### Preparation of Intermediate 10.4

### 6-bromo-8-chloro-N′-(2,2-difluoroacetyl)imidazo[1,2-a]pyridine-3-carbohydrazide

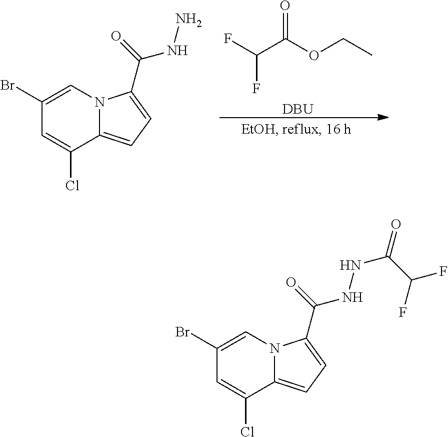

To a solution of 6-bromo-8-chloroimidazo[1,2-a]pyridine-3-carbohydrazide (1.36 g, 4.71 mmol) and DBU (1.43 g, 9.42 mmol) in EtOH (15 mL) was added ethyl 2,2-difluoroacetate (5.84 g, 47.10 mmol). The mixture was stirred at 105° C. for 16 h. The mixture was concentrated in vacuum to give a residue, which was purified by flash silica gel chromatography (ISCO®; 12 g SepaFlash® Silica Flash Column, eluent 0-50% EtOAc/PE gradient @36 mL/min) to give the product 6-bromo-8-chloro-N′-(2,2-difluoroacetyl)imidazo[1,2-a]pyridine-3-carbohydrazide (386 mg, 1.05 mmol) as a yellow solid.

RT 0.885 min (method 1); m/z 368.0 (M+H)⁺ (ESI⁺); ¹H NMR (400 MHz, DMSO-d₆) δ10.94 (s, 1H), 10.56 (s, 1H), 9.66 (s, 1H), 7.75 (d, J=4.8 Hz, 1H), 7.53 (d, J=1.2 Hz, 1H), 6.80 (d, J=4.00 Hz, 1H), 6.47 (t, J=53.2 Hz, 1H).

### Preparation of Intermediate 10.5

### 2-(6-bromo-8-chloroindolizin-3-yl)-5-(difluoromethyl)-1,3,4-thiadiazole

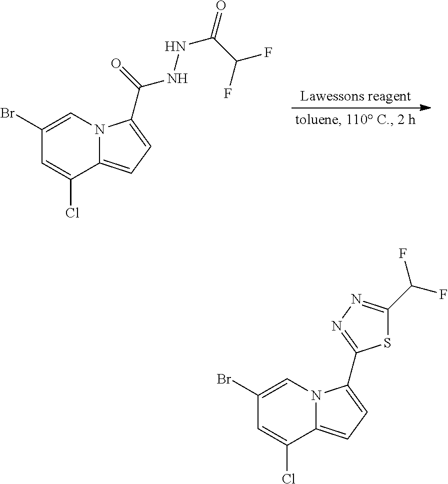 [SMILES: O=C(NNC(=O)C(F)F)c1ccc2c(Cl)cc(Br)cn12 ; "Lawessons reagent" ; FC(F)c1nnc(-c2ccc3c(Cl)cc(Br)cn23)s1]

To a solution of 6-bromo-8-chloro-N′-(2,2-difluoroacetyl)indolizine-3-carbohydrazide (386.0 mg, 1.05 mmol) in toluene (5 mL) was added Lawessons reagent (468.5 mg, 1.16 mmol). The mixture was stirred at 110° C. for 2 h. The mixture was concentrated in vacuum to give a residue, which was purified by flash silica gel chromatography (ISCO®; 4 g SepaFlash® Silica Flash Column, eluent 0-100% EtOAc/PE gradient @10 mL/min) to give the product 2-(6-bromo-8-chloroindolizin-3-yl)-5-(difluoromethyl)-1,3,4-thiadiazole (210.0 mg, 0.58 mmol) as a yellow solid.

RT 1.086 min (method 1); m/z 365.9 (M+H)⁺ (ESI⁺); ¹H NMR (400 MHz, DMSO-d₆) δ9.75 (s, 1H), 7.51-7.82 (m, 3H), 6.94 (d, J=4.0 Hz, 1H).

### Preparation of Intermediate 10.6

### 2-(6-(benzylthio)-8-chloroindolizin-3-yl)-5-(difluoromethyl)-1,3,4-thiadiazole

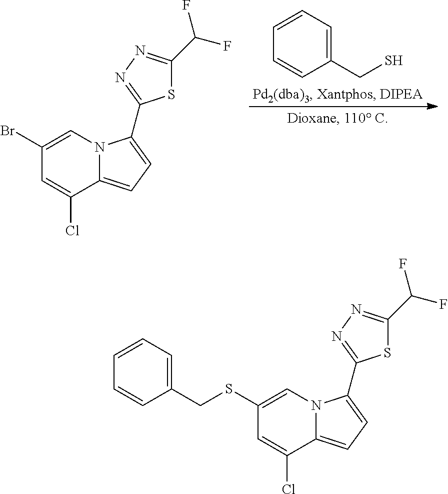

A mixture of 2-(6-bromo-8-chloroindolizin-3-yl)-5-(difluoromethyl)-1,3,4-thiadiazole (210.0 mg, 575.98 μmol), phenylmethanethiol (71.5 mg, 575.98 μmol), Pd₂(dba)₃ (52.7 mg, 57.60 μmol), DIEA (148.9 mg, 1.15 mmol) and Xantphos (66.7 mg, 115.20 μmol) in dioxane (3 mL) which was degassed with nitrogen and heated to 90° C. for 2 hours under a nitrogen atmosphere. The mixture was concentrated in vacuum to give a residue, which was purified by flash silica gel chromatography (ISCO®; 4 g SepaFlash® Silica Flash Column, eluent 0-50% EtOAc/PE gradient @10 mL/min) to give the product 2-(6-(benzylthio)-8-chloroindolizin-3-yl)-5-(difluoromethyl)-1,3,4-thiadiazole (200.0 mg, 490.33 μmol) as a yellow solid.

RT 1.156 min (method 1); m/z 408.1 (M+H)⁺ (ESI⁺); ¹H NMR (400 MHz, DMSO-d₆) δ 7.15-7.84 (m, 9H), 6.87 (d, J=4.52 Hz, 1H), 4.27 (s, 2H).

### Preparation of Intermediate 10.7

### 2-(6-(benzylsulfinyl)-8-chloro-1-iodoindolizin-3-yl)-5-(difluoromethyl)-1,3,4-thiadiazole

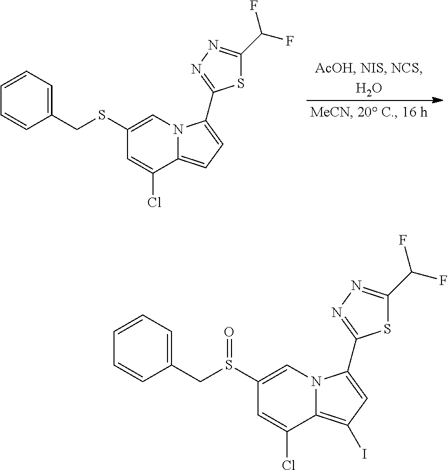

To the mixture of 2-(6-(benzylthio)-8-chloroindolizin-3-yl)-5-(difluoromethyl)-1,3,4-thiadiazole (150.0 mg, 367.75 μmol) in MeCN (0.5 mL) was added NIS (182.0 mg, 809.05 μmol), AcOH (22.1 mg, 367.75 μmol) and H₂O (6.6 mg, 367.75 μmol). The mixture was stirred at 20° C. for 2 h. The mixture was concentrated in vacuum to give a residue, which was purified by preparative HPLC (column: Waters Xbridge 150*50 mm*10 μm; mobile phase: A: 1 mM aqueous solution of NH₄HCO₃, B: MeCN; B %: 49%-79%, 11 min) to give the product 2-(6-(benzylsulfinyl)-8-chloro-1-iodoindolizin-3-yl)-5-(difluoromethyl)-1,3,4-thiadiazole (100.0 mg, 181.89 μmol) as a yellow solid.

RT 1.054 min (method 1); m/z 550.0 (M+H)⁺ (ESI⁺).

### Preparation of Intermediate 10.8

### 8-chloro-3-(5-(difluoromethyl)-1,3,4-thiadiazol-2-yl)-1-iodoindolizine-6-sulfonyl chloride

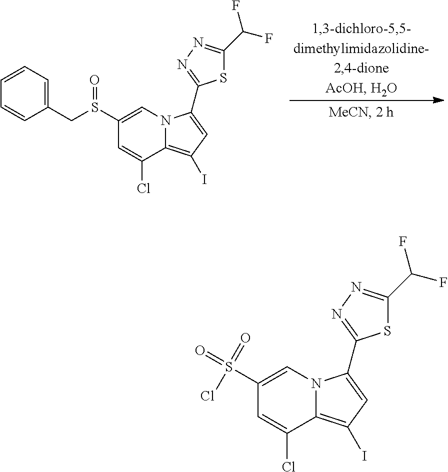 [SMILES: O=S(Cc1ccccc1)c1cc(Cl)c2c(I)cc(-c3nnc(C(F)F)s3)n2c1 ; "1,3-dichloro-5,5-dimethylimidazolidine-2,4-dioneAcOH, H2O" ; O=S(=O)(Cl)c1cc(Cl)c2c(I)cc(-c3nnc(C(F)F)s3)n2c1]

To a mixture of 2-(6-(benzylsulfinyl)-8-chloro-1-iodoindolizin-3-yl)-5-(difluoromethyl)-1,3,4-thiadiazole (20.0 mg, 36.38 μmol) in MeCN (2 mL) was added H₂O (0.7 mg, 36.38 μmol) and AcOH (4.4 mg, 72.76 μmol) at 0° C. The mixture was stirred at 0° C. for 10 min. 1,3-dichloro-5,5-dimethylimidazolidine-2,4-dione (14.3 mg, 72.76 μmol) was added and the mixture was stirred at 0° C. for 2 h. The mixture was diluted with THE (3 mL) and dried over Na₂SO₄. After filtration, the filtrate was concentrated under vacuum to give crude 8-chloro-3-(5-(difluoromethyl)-1,3,4-thiadiazol-2-yl)-1-iodoindolizine-6-sulfonyl chloride (20.0 mg, 19.60 μmol) as light brown oil. The crude product used for next step without further purification.

### Preparation of Intermediate 10.9

### 8-chloro-N-(1-cyanocyclopropyl)-3-(5-(difluoromethyl)-1,3,4-thiadiazol-2-yl)-1-iodoindolizine-6-sulfonamide

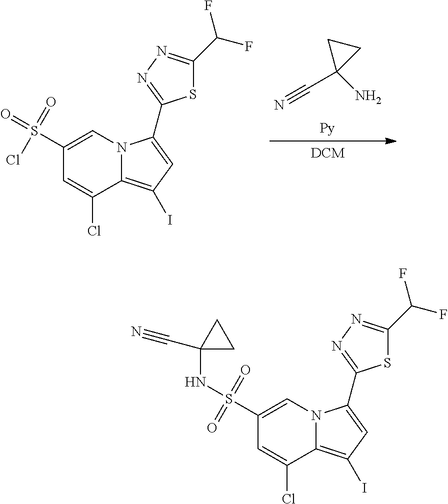 [SMILES: O=S(=O)(Cl)c1cc(Cl)c2c(I)cc(-c3nnc(C(F)F)s3)n2c1 ; N#CC1(N)CC1 ; N#CC1(NS(=O)(=O)c2cc(Cl)c3c(I)cc(-c4nnc(C(F)F)s4)n3c2)CC1]

To a mixture of 8-chloro-3-(5-(difluoromethyl)-1,3,4-thiadiazol-2-yl)-1-iodoindolizine-6-sulfonyl chloride (20.0 mg, 39.80 μmol) in pyridine (1 mL) at 0° C. was added 1-aminocyclopropane-1-carbonitrile (16.1 mg, 196.05 μmol, HCl salt) in THF (1 mL) over 1 min. The mixture was stirred at 0° C. for 1 h. The mixture was concentrated under vacuum to give a residue. The residue was dissolved in DCM (20 mL) and washed with brine (20 mL). The organic phase was dried over Na₂SO₄, filtered and concentrated under vacuum to give a residue, which was purified by preparative HPLC (column: Unisil 3-100 C18 Ultra 150*50 mm*3 μm; mobile phase: A: 0.225% formic acid in water, B: MeCN; B %: 45%-75%, 10 min) to give the product 8-chloro-N-(1-cyanocyclopropyl)-3-(5-(difluoromethyl)-1,3,4-thiadiazol-2-yl)-1-iodoindolizine-6-sulfonamide (15.0 mg, 24.13 μmol, 89.4% purity) as a yellow solid.

RT 0.890 min (method 1); m/z 555.7 (M+H)⁺ (ESI⁺).

### Preparation of Example 10

### 8-chloro-N-(1-cyanocyclopropyl)-3-(5-(difluoromethyl)-1,3,4-thiadiazol-2-yl)indolizine-6-sulfonamide

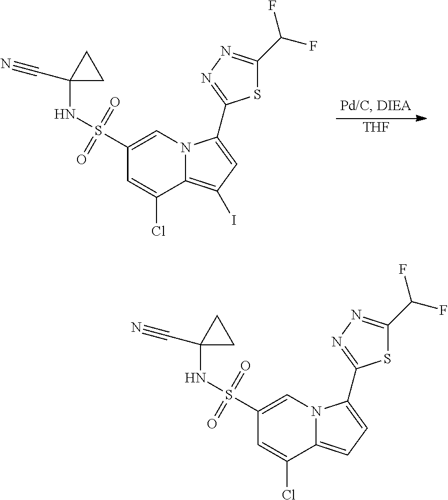 [SMILES: N#CC1(NS(=O)(=O)c2cc(Cl)c3c(I)cc(-c4nnc(C(F)F)s4)n3c2)CC1 ; N#CC1(NS(=O)(=O)c2cc(Cl)c3ccc(-c4nnc(C(F)F)s4)n3c2)CC1 ; "THF"]

To a mixture of 8-chloro-N-(1-cyanocyclopropyl)-3-(5-(difluoromethyl)-1,3,4-thiadiazol-2-yl)-1-iodoindolizine-6-sulfonamide (15.0 mg, 26.99 μmol) in THE (5 mL) was added DIEA (3.5 mg, 26.99 μmol) and Pd/C (5.0 mg, 26.99 μmol, 10% purity) at 20° C. The mixture was stirred at 20° C. under a hydrogen atmosphere (15 Psi) for 2 h. The mixture was filtered and concentrated under vacuum to give a residue, which was purified by preparative HPLC (column: Phenomenex C18 75*30 mm*3 um; mobile phase: A: 0.225% formic acid in water, B: MeCN; B %: 40%-70%, 7 min) to give the product 8-chloro-N-(1-cyanocyclopropyl)-3-[5-(difluoromethyl)-1,3,4-thiadiazol-2-yl]indolizine-6-sulfonamide (1.9 mg, 3.75 μmol, FA salt) as a yellow solid.

RT 0.899 min (method 1); m/z 430.0 (M+H)⁺ (ESI⁺); ¹H NMR (400 MHz, DMSO-d₆) δ10.06 (s, 1H), 8.37 (s, 2H), 7.91 (d, J=4.4 Hz, 1H), 7.68 (t, J=53.2 Hz, 1H), 7.40 (d, J=1.2 Hz, 1H), 6.98 (d, J=4.4 Hz, 1H), 1.22-1.18 (m, 2H), 1.11-1.15 (m, 2H).

### Preparation of Intermediate 11.1

### ethyl 6-(benzylthio)-8-chloroimidazo[1,2-a]pyridine-3-carboxylate

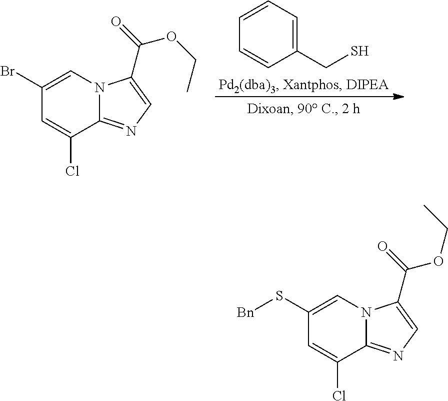

To a mixture of ethyl 6-bromo-8-chloro-imidazo[1,2-a]pyridine-3-carboxylate (1.50 g, 4.94 mmol, Intermediate 1.2), phenylmethanethiol (0.61 g, 4.94 mmol, 579.03 μL), DIPEA (1.28 g, 9.88 mmol, 1.72 mL) and Xantphos (0.57 g, 988.34 μmol) in dioxane (15 mL) was added Pd₂(dba)₃ (0.45 g, 494.17 μmol). The mixture was stirred at 90° C. for 2 h under a nitrogen atmosphere. The mixture was cooled to room temperature, quenched by H₂O (100 mL) and extracted with EtOAc (100 mL; 2×). The combined organic layers were washed by brine (100 mL), dried over Na₂SO₄, filtered. The filtrate was concentrated under reduced pressure to give a residue which was purified by flash silica gel chromatography (ISCO®; 40 g SepaFlash® Silica Flash Column, eluent 60-100% EtOAc/PE gradient @100 mL/min) to give a crude product which was further purified by reverse phase flash (solvent for sample dissolution about 10 g of sample dissolved in 10 mL of MeOH, (column: I.D.95 mm*H365 mm Welch Ultimate XB_C18 20-40 μm; 120 A; mobile phase: A: 0.225% formic acid in water, B: MeCN; B %: 5-90% 40 min; 90% 10 min; 200 ml/min)). The mixture was concentrated under reduced pressure to remove most of MeCN, then the resulting mixture was neutralized with NaHCO₃ to pH=8, and extracted with EtOAc (100 mL; 2×). The combined organic layers were washed by brine (100 mL; 2×), dried over Na₂SO₄, filtered. The filtrate was concentrated under reduced pressure to give the product ethyl 6-benzylsulfanyl-8-chloro-imidazo[1,2-a]pyridine-3-carboxylate (1.5 g, 4.32 mmol) as a brown solid. (This reaction was conducted as two batches in parallel, and purified together.)

¹H NMR (DMSO-d₆, 400 MHz): 8.99 (d, J=1.6 Hz, 1H), 8.31 (s, 1H), 7.85 (d, J=1.6 Hz, 1H), 7.33-7.18 (m, 5H), 4.41-4.32 (m, 2H), 4.29 (s, 2H), 1.34 (t, J=7.2 Hz, 3H).

### Preparation of Intermediate 11.2

### ethyl 8-chloro-6-(chlorosulfonyl)imidazo[1,2-a]pyridine-3-carboxylate

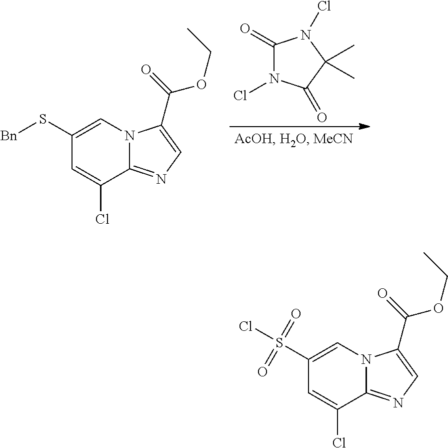

To a mixture of ethyl 6-benzylsulfanyl-8-chloro-imidazo[1,2-a]pyridine-3-carboxylate (200.0 mg, 576.65 μmol), AcOH (58.9 mg, 980.31 μmol, 56.1 μL) and H₂O (10.4 mg, 576.65 μmol, 10.4 μL) in MeCN (2 mL) was added 1,3-dichloro-5,5-dimethyl-imidazolidine-2,4-dione (227.2 mg, 1.15 mmol) at 0° C. The mixture was stirred at 0° C. for 0.5 h. The mixture was diluted with THE (15 mL), dried over Na₂SO₄ and filtered. The filtrate was concentrated under reduced pressure to give the product ethyl 8-chloro-6-chlorosulfonyl-imidazo[1,2-a]pyridine-3-carboxylate (150.0 mg, 464.18 μmol) as a yellow oil and this crude product was used into next step directly without purification.

### Preparation of Example 11

### ethyl 8-chloro-6-(N-(1-cyanocyclopropyl)sulfamoyl)imidazo[1,2-a]pyridine-3-carboxylate

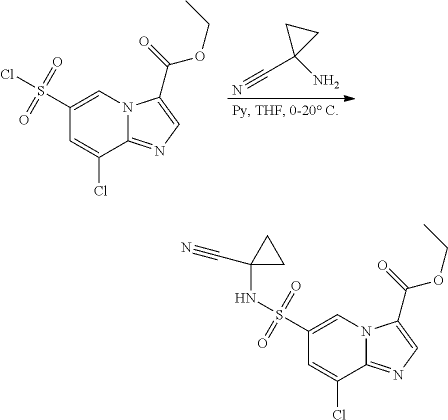 [SMILES: CCOC(=O)c1cnc2c(Cl)cc(S(=O)(=O)Cl)cn12 ; N#CC1(N)CC1 ; CCOC(=O)c1cnc2c(Cl)cc(S(=O)(=O)NC3(C#N)CC3)cn12]

To the mixture of 1-aminocyclopropanecarbonitrile (55.0 mg, 464.18 μmol, HCl salt) in pyridine (392.0 mg, 4.96 mmol, 0.4 mL) was added dropwise a solution of ethyl 8-chloro-6-chlorosulfonyl-imidazo[1,2-a]pyridine-3-carboxylate (150.0 mg, 464.18 μmol) in THE (1 mL) at 0° C. The mixture was stirred at 20° C. for 2 h before it was quenched by H₂O (30 mL) and extracted with EtOAc (20 mL; 3×). The combined organic layers were washed by brine (30 mL), dried over Na₂SO₄, filtered and concentrated. The residue was purified by preparative HPLC (column: Unisil 3-100 C18 Ultra 150*50 mm*3 μm; mobile phase: A: 0.225% formic acid in water, B: MeCN; 30%-60%, 10 min) to give the product ethyl 8-chloro-6-[(1-cyanocyclopropyl)sulfamoyl]imidazo[1,2-a]pyridine-3-carboxylate (4.1 mg, 9.76 μmol, 99% purity, FA salt) as a light yellow solid.

RT 0.643 min (method 1); m/z 368.8 (M+H)⁺ (ES⁺); ¹H NMR (DMSO-d₆, 400 MHz): 9.64 (d, J=1.2 Hz, 1H), 8.47 (s, 1H), 8.26 (s, 1H), 7.88 (d, J=1.2 Hz, 1H), 4.51-4.35 (m, 2H), 1.40-1.32 (m, 5H), 1.28-1.18 (m, 2H).

### Preparation of Example 12

### ethyl 6-(N-(1-cyanocyclopropyl)sulfamoyl)-8-(4-(dimethylcarbamoyl)piperazin-1-yl)imidazo[1,2-a]pyridine-3-carboxylate

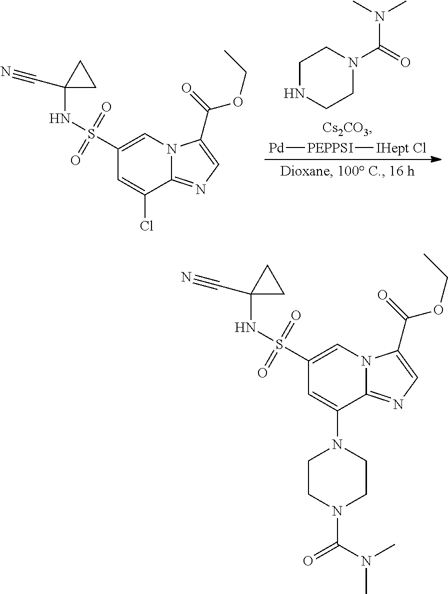

To a mixture of ethyl 8-chloro-6-[(1-cyanocyclopropyl)sulfamoyl]imidazo[1,2-a]pyridine-3-carboxylate (10.0 mg, 24.11 μmol, FA salt) and Cs₂CO₃ (23.6 mg, 72.32 μmol) in dioxane (0.5 mL) was added Pd-PEPPSI-IHeptCl (2.4 mg, 2.41 μmol) in a glove box. The mixture was stirred at 100° C. for 3 h under an argon atmosphere. The mixture was concentrated under reduced pressure to give a residue which was purified by preparative HPLC (column: Unisil 3-100 C18 Ultra 150*50 mm*3 μm; mobile phase: A: 0.225% formic acid in water, B: MeCN; B %: 28%-58%, 10 min) to give the product ethyl 6-[(1-cyanocyclopropyl)sulfamoyl]-8-[4-(dimethylcarbamoyl)piperazin-1-yl]imidazo[1,2-a]pyridine-3-carboxylate (1.6 mg, 2.83 μmol, FA salt) as an off-white solid.

RT 0.687 min (method 1); m/z 490.1 (M+H)⁺ (ES⁺); ¹H NMR (DMSO-d₆, 400 MHz): 9.33 (d, J=1.2 Hz, 1H), 8.37 (s, 1H), 8.32 (s, 1H), 6.97 (d, J=1.2 Hz, 1H), 4.47-4.32 (m, 2H), 3.63-3.56 (m, 4H), 3.35 (br d, J=4.8 Hz, 4H), 2.80 (s, 6H), 1.37 (t, J=7.2 Hz, 3H), 1.33-1.27 (m, 2H), 1.25-1.18 (m, 2H).

### Preparation of Intermediate 13.1

### 8-chloro-3-(5-(difluoromethyl)-1,3,4-thiadiazol-2-yl)-1-iodo-N-(1-methylcyclopropyl)imidazo[1,5-a]pyridine-6-sulfonamide

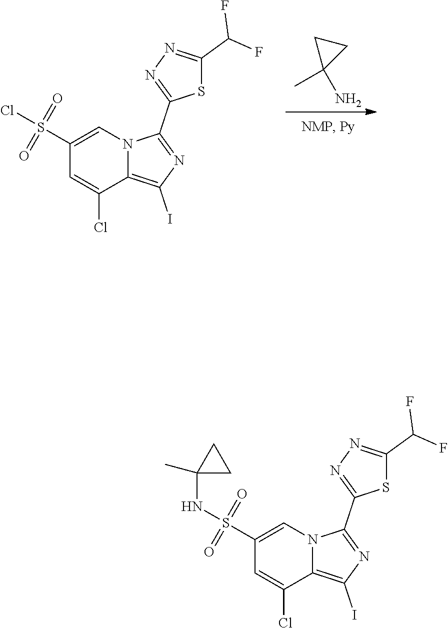

To a mixture of 1-methylcyclopropan-1-amine (37.80 mg, 531.49 μmol) in pyridine (1 mL) and NMP (N-methyl-2-pyrrolidon) (1 mL) at 0° C. was added 8-chloro-3-(5-(difluoromethyl)-1,3,4-thiadiazol-2-yl)-1-iodoimidazo[1,5-a]pyridine-6-sulfonyl chloride (90 mg, 176.09 μmol) in MeCN (2 mL). The reaction was stirred at 0° C. for 50 min. The reaction mixture was quenched with water (10 mL) and extracted with EtOAc (10 mL; 2×). The organic phase was collected, dried over Na₂SO₄, filtered and concentrated under vacuum to give a residue which was purified by preparative TLC (PE:EtOAc=3:1) to give the product 8-chloro-3-(5-(difluoromethyl)-1,3,4-thiadiazol-2-yl)-1-iodo-N-(1-methylcyclopropyl)imidazo[1,5-a]pyridine-6-sulfonamide (25 mg, 45.81 μmol, 26.01% yield) as a light yellow solid.

It is noted that it cannot be excluded that be excluded that the dichloro compound 1,8-dichloro-3-(5-(difluoromethyl)-1,3,4-thiadiazol-2-yl)-N-(1-methylcyclopropyl)imidazo[1,5-a]pyridine-6-sulfonamide was also formed in this process.

RT 0.510 min (method 3); m/z 545.8 (M+H)⁺ (ESI⁺)

### Preparation of Intermediate 13.2

### 8-chloro-3-(5-(difluoromethyl)-1,3,4-thiadiazol-2-yl)-N-(1-methylcyclopropyl)imidazo[1,5-a]pyridine-6-sulfonamide

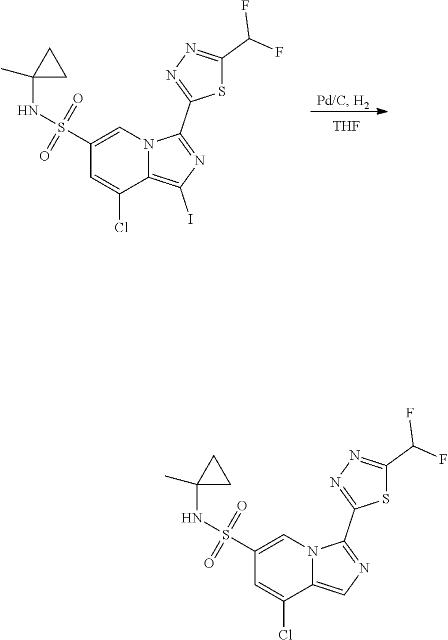 [SMILES: CC1(NS(=O)(=O)c2cc(Cl)c3c(I)nc(-c4nnc(C(F)F)s4)n3c2)CC1 ; CC1(NS(=O)(=O)c2cc(Cl)c3cnc(-c4nnc(C(F)F)s4)n3c2)CC1 ; "THF"]

To a mixture of 8-chloro-3-(5-(difluoromethyl)-1,3,4-thiadiazol-2-yl)-1-iodo-N-(1-methylcyclopropyl)imidazo[1,5-a]pyridine-6-sulfonamide (25 mg, 45.81 μmol) in tetrahydrofuran (3 mL) was added Pd/C (5 mg, 10% purity). The reaction was degassed with H₂ (15 Psi) three times, then the reaction was stirred at 20° C. for 3 h. The reaction mixture was filtered and the filtrate was concentrated under vacuum to give the product 8-chloro-3-(5-(difluoromethyl)-1,3,4-thiadiazol-2-yl)-N-(1-methylcyclopropyl)imidazo[1,5-a]pyridine-6-sulfonamide (20 mg, 30.96 μmol, 67.59% yield, 65% purity) as a brown solid.

It is noted that it cannot be excluded that the dichloro compound 1,8-dichloro-3-(5-(difluoromethyl)-1,3,4-thiadiazol-2-yl)-N-(1-methylcyclopropyl)imidazo[1,5-a]pyridine-6-sulfonamide was also formed in this process (see intermediate Examples 18c and 18d).

RT 0.468 min (method 3); m/z 420.0 (M+H)⁺ (ESI⁺)

### Preparation of Example 13

### 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperazine-1-carboxamide

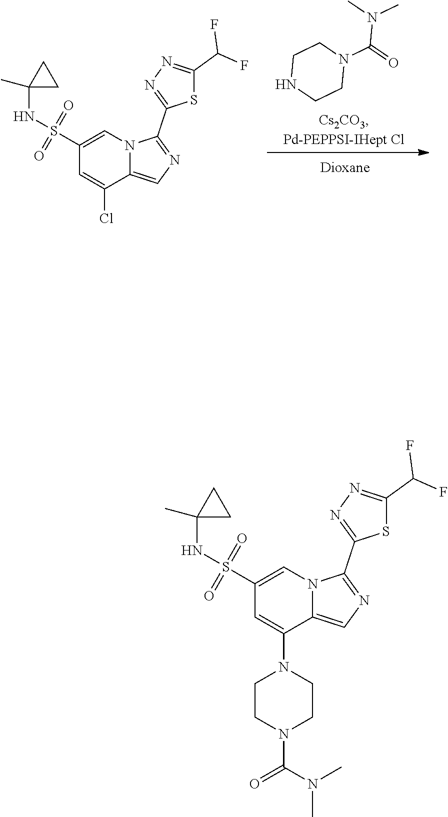 [SMILES: CC1(NS(=O)(=O)c2cc(Cl)c3cnc(-c4nnc(C(F)F)s4)n3c2)CC1 ; CN(C)C(=O)N1CCNCC1 ; "Dioxane" ; CN(C)C(=O)N1CCN(c2cc(S(=O)(=O)NC3(C)CC3)cn3c(-c4nnc(C(F)F)s4)ncc23)CC1]

To a mixture of 8-chloro-3-(5-(difluoromethyl)-1,3,4-thiadiazol-2-yl)-N-(1-methylcyclopropyl)imidazo[1,5-a]pyridine-6-sulfonamide (15 mg, 35.73 μmol) and N,N-dimethylpiperazine-1-carboxamide (11.23 mg, 71.45 μmol) in degassed 1,4-dioxane (1 mL) under nitrogen was added Pd-PEPPSI-IHept Cl (3.48 mg, 3.57 μmol) and Cs₂CO₃ (23.28 mg, 71.45 μmol). The mixture was stirred at 100° C. for 16 h. The mixture was filtered and the filtrate was concentrated under vacuum to give a residue. The residue was purified by preparative TLC (EtOAc:PE=2:1) to give 5 mg crude product, which was further purified by preparative HPLC (column: Phenomenex luna C18 150*25 mm*10 μm; mobile phase: A: 0.225% formic acid in water, B: MeCN; B %: 37%-67%, 10 min) and lyophilized directly to give the product 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperazine-1-carboxamide (1.03 mg, 1.85 μmol, 5.19% yield) as a light yellow solid.

RT 0.435 min (method 3); m/z 541.2 (M+H)⁺ (ESI⁺); ¹H NMR (CHLOROFORM-d, 400 MHz) 9.88 (s, 1H), 7.72 (s, 1H), 7.08 (t, J=53.6 Hz, 1H), 6.66 (d, J=1.1 Hz, 1H), 5.06 (s, 1H), 3.53-3.50 (m, 4H), 3.36-3.34 (m, 4H), 2.91 (s, 6H), 1.39 (s, 3H), 0.95-0.92 (m, 2H), 0.62-0.60 (m, 2H).

### Preparation of Intermediate 14.1

### 8-chloro-3-(5-(difluoromethyl)-1,3,4-thiadiazol-2-yl)-N-(1-(fluoromethyl)cyclopropyl)-1-iodoimidazo[1,5-a]pyridine-6-sulfonamide

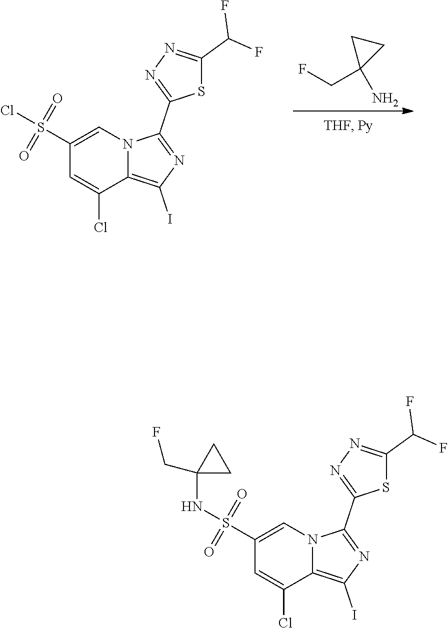

To a mixture of 1-(fluoromethyl)cyclopropan-1-amine (18.43 mg, 146.74 μmol, HCl salt) in pyridine (1 mL) and THE (1 mL) at 0° C. was added 8-chloro-3-(5-(difluoromethyl)-1,3,4-thiadiazol-2-yl)-1-iodoimidazo[1,5-a]pyridine-6-sulfonyl chloride (50 mg, 97.83 μmol) in MeCN (2 mL) and the mixture was stirred at 0° C. for 1 h. The mixture was quenched by water (10 mL) and extracted with EtOAc (20 mL; 2×). The organic phase was collected, dried over Na₂SO₄, filtered and concentrated under vacuum to give a residue which was purified by preparative TLC (PE:EtOAc=3:1) to give the product 8-chloro-3-(5-(difluoromethyl)-1,3,4-thiadiazol-2-yl)-N-(1-(fluoromethyl)cyclopropyl)-1-iodoimidazo[1,5-a]pyridine-6-sulfonamide (39 mg, 52.58 μmol, 53.74% yield, 76% purity) as a light yellow solid.

RT 0.487 min (method 3); m/z 463.9 (M+H)⁺ (ESI⁺)

### Preparation of Intermediate 14.2

### 8-chloro-3-(5-(difluoromethyl)-1,3,4-thiadiazol-2-yl)-N-(1-(fluoromethyl)cyclopropyl)imidazo[1,5-a]pyridine-6-sulfonamide

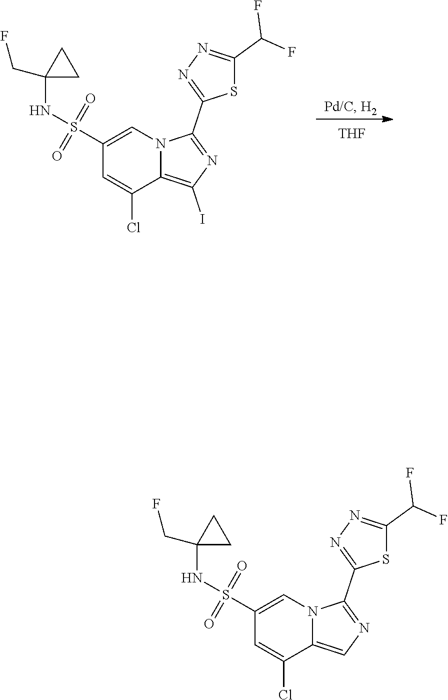 [SMILES: O=S(=O)(NC1(CF)CC1)c1cc(Cl)c2c(I)nc(-c3nnc(C(F)F)s3)n2c1 ; O=S(=O)(NC1(CF)CC1)c1cc(Cl)c2cnc(-c3nnc(C(F)F)s3)n2c1 ; "THF"]

To a mixture of 8-chloro-3-(5-(difluoromethyl)-1,3,4-thiadiazol-2-yl)-N-(1-(fluoromethyl)cyclopropyl)-1-iodoimidazo[1,5-a]pyridine-6-sulfonamide (35 mg, 62.08 μmol) in THF (5 mL) was added Pd/C (5 mg, 10% purity). The reaction was degassed with H₂ (15 Psi) three times, then the reaction was stirred at 20° C. for 3 h. The reaction mixture was filtered and the filtrate was concentrated under vacuum to give crude product which was purified by preparative TLC (PE:EtOAc=1:1) to give the product 8-chloro-3-(5-(difluoromethyl)-1,3,4-thiadiazol-2-yl)-N-(1-(fluoromethyl)cyclopropyl)imidazo[1,5-a]pyridine-6-sulfonamide (10 mg, 12.49 μmol, 20.12% yield, 54.7% purity) as a brown solid.

RT 0.441 min (method 3); m/z 437.9 (M+H)⁺ (ESI⁺)

### Preparation of Example 14

### 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide

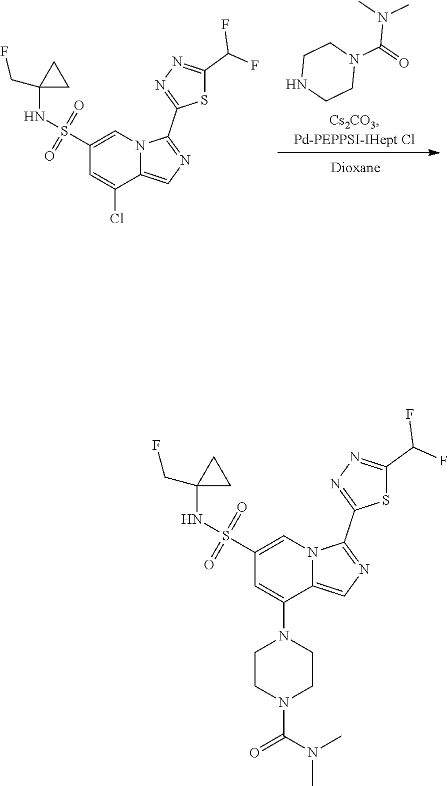 [SMILES: O=S(=O)(NC1(CF)CC1)c1cc(Cl)c2cnc(-c3nnc(C(F)F)s3)n2c1 ; CN(C)C(=O)N1CCNCC1 ; "Dioxane" ; CN(C)C(=O)N1CCN(c2cc(S(=O)(=O)NC3(CF)CC3)cn3c(-c4nnc(C(F)F)s4)ncc23)CC1]

To a mixture of 8-chloro-3-(5-(difluoromethyl)-1,3,4-thiadiazol-2-yl)-N-(1-(fluoromethyl)cyclopropyl)imidazo[1,5-a]pyridine-6-sulfonamide (10 mg, 22.84 μmol) and N,N-dimethylpiperazine-1-carboxamide (7.18 mg, 45.68 μmol) in degassed 1,4-dioxane (1 mL) under nitrogen was added Pd-PEPPSI-IHept Cl (2.22 mg, 2.28 μmol) and Cs₂CO₃ (14.88 mg, 45.68 μmol) and the mixture was stirred at 100° C. for 1.5 h. The reaction mixture was filtered and the filtrate was concentrated under vacuum to give a residue which was purified by preparative HPLC (column: Phenomenex luna C18 150*25 mm*10 μm; mobile phase: A: 0.225% formic acid in water, B: MeCN; B %: 36%-66%, 10 min) and lyophilized directly to give the product 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide (2.88 mg, 5.16 μmol, 22.57% yield) as a light yellow solid.

RT 0.417 min (method 3); m/z 559.1 (M+H)⁺ (ESI⁺); ¹H NMR (CHLOROFORM-d, 400 MHz): 9.89 (s, 1H), 7.73 (s, 1H), 7.08 (t, J=53.6 Hz, 1H), 6.65 (s, 1H), 5.50 (s, 1H), 4.28 (d, J=48.4 Hz, 2H), 3.53-3.51 (m, 4H), 3.37-3.34 (m, 4H), 2.91 (s, 6H), 1.16-1.15 (m, 2H), 0.88-0.86 (m, 2H).

### Preparation of Intermediate 18.1

### 2-(6-(benzylthio)-8-chloro-1-iodoimidazo[1,5-a]pyridin-3-yl)-5-(difluoromethyl)-1,3,4-thiadiazole

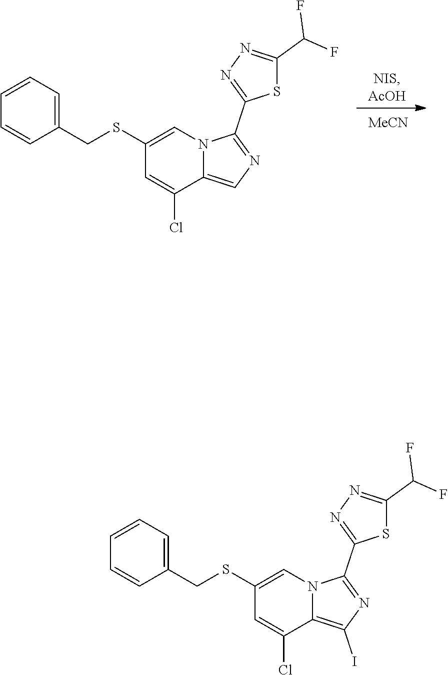 [SMILES: "MeCN" ; FC(F)c1nnc(-c2ncc3c(Cl)cc(SCc4ccccc4)cn23)s1 ; FC(F)c1nnc(-c2nc(I)c3c(Cl)cc(SCc4ccccc4)cn23)s1]

To a mixture of 2-(6-(benzylthio)-8-chloroimidazo[1,5-a]pyridin-3-yl)-5-(difluoromethyl)-1,3,4-thiadiazole (900 mg, 2.20 mmol) in MeCN (10 mL) were added NIS (1.39 g, 6.16 mmol) and AcOH (396.54 mg, 6.60 mmol, 377.66 μL). The reaction mixture was warmed to 25° C., stirred for 16 and filtered. The cake was washed with MeCN (5 mL). Then, the filtrate was concentrated under vacuum to give the product 2-(6-(benzylthio)-8-chloro-1-iodoimidazo[1,5-a]pyridin-3-yl)-5-(difluoromethyl)-1,3,4-thiadiazole (850 mg, 1.59 mmol) as a yellow solid.

RT 0.607 min (method 3); m/z 534.8 (M+H)⁺ (ESI⁺); ¹H NMR (DMSO-d₆, 400 MHz): 9.33 (s, 1H), 7.67 (t, J=50.8 Hz, 1H), 7.50 (s, 1H), 7.38 (d, 2H), 7.29 (t, J=7.2 Hz, 2H), 7.20 (t, J=7.2 Hz, 1H), 4.34 (s, 2H)

### Preparation of Intermediate 18.2

### 8-chloro-3-(5-(difluoromethyl)-1,3,4-thiadiazol-2-yl)-1-iodoimidazo[1,5-a]pyridine-6-sulfonyl chloride and 1,8-dichloro-3-(5-(difluoromethyl)-1,3,4-thiadiazol-2-yl)imidazo[1,5-a]pyridine-6-sulfonyl chloride

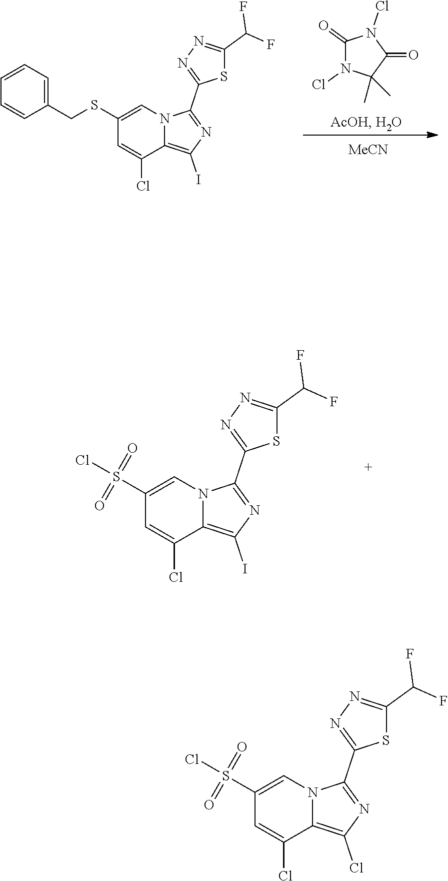 [SMILES: "MeCN" ; FC(F)c1nnc(-c2nc(I)c3c(Cl)cc(SCc4ccccc4)cn23)s1 ; CC1(C)C(=O)N(Cl)C(=O)N1Cl ; O=S(=O)(Cl)c1cc(Cl)c2c(I)nc(-c3nnc(C(F)F)s3)n2c1 ; O=S(=O)(Cl)c1cc(Cl)c2c(Cl)nc(-c3nnc(C(F)F)s3)n2c1]

To a mixture of 2-(6-(benzylthio)-8-chloro-1-iodoimidazo[1,5-a]pyridin-3-yl)-5-(difluoromethyl)-1,3,4-thiadiazole (850.00 mg, 1.59 mmol) in MeCN (12 mL) at 0° C. were added AcOH (190.90 mg, 3.18 mmol, 181.81 μL), H₂O (57.27 mg, 3.18 mmol, 57.27 μL) and 1,3-dichloro-5,5-dimethylimidazolidine-2,4-dione (626.31 mg, 3.18 mmol). Then, the mixture was stirred at 0° C. for 1 h. The mixture was used for next step directly without further work-up.

RT 0.510 min (method 3); m/z 510.8 (M+H)⁺ (ESI⁺);

### Preparation of Example 18a and 18b

### 8-chloro-3-(5-(difluoromethyl)-1,3,4-thiadiazol-2-yl)-1-iodo-N-(1-methylcyclopropyl)imidazo[1,5-a]pyridine-6-sulfonamide and 1,8-dichloro-3-(5-(difluoromethyl)-1,3,4-thiadiazol-2-yl)-N-(1-methylcyclopropyl)imidazo[1,5-a]pyridine-6-sulfonamide

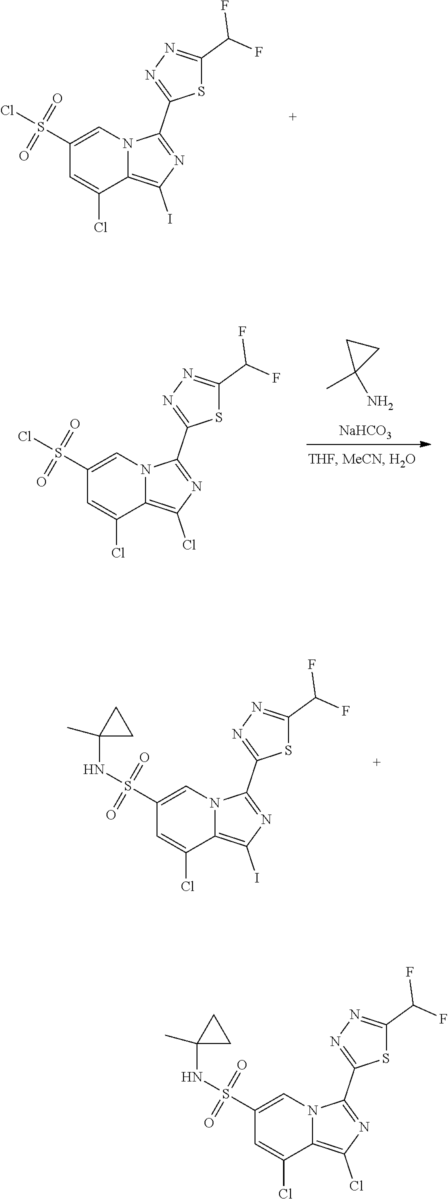

To a solution of 1-methylcyclopropanamine (354.84 mg, 4.99 mmol) in THE (10 mL) and H₂O (5 mL) was added NaHCO₃ (1.40 g, 16.63 mmol) and the mixture was cooled to 0° C. Then, the mixture of 8-chloro-3-(5-(difluoromethyl)-1,3,4-thiadiazol-2-yl)-1-iodoimidazo[1,5-a]pyridine-6-sulfonyl chloride and 1,8-dichloro-3-(5-(difluoromethyl)-1,3,4-thiadiazol-2-yl)imidazo[1,5-a]pyridine-6-sulfonyl chloride (850 mg, crude) in MeCN (12 mL) from previous step was added dropwise. The resulting reaction mixture was stirred at 0° C. for 1 h, quenched with H₂O (30 mL) and extracted with EtOAc (60 mL; 3×). The combined organic layer was washed with brine (50 mL; 3×), dried over Na₂SO₄, filtered and the filtrate was concentrated under vacuum. The crude product was purified by flash silica gel chromatography (ISCO®; 12 g SepaFlash® Silica Flash Column, Eluent of 0˜50% EtOAc/Petroleum ether gradient @30 mL/min) to give the product as a mixture of 8-chloro-3-(5-(difluoromethyl)-1,3,4-thiadiazol-2-yl)-1-iodo-N-(1-methylcyclopropyl)imidazo[1,5-a]pyridine-6-sulfonamide and 1,8-dichloro-3-(5-(difluoromethyl)-1,3,4-thiadiazol-2-yl)-N-(1-methylcyclopropyl)imidazo[1,5-a]pyridine-6-sulfonamide (430 mg, crude) as a yellow solid.

RT 0.537 min (method 3); m/z 545.8 (M+H)⁺ (ESI⁺), m/z 453.9 (M+H)⁺ (ESI⁺);

### Preparation of Examples 18c and 18d

### 8-chloro-3-(5-(difluoromethyl)-1,3,4-thiadiazol-2-yl)-N-(1-methylcyclopropyl)imidazo[1,5-a]pyridine-6-sulfonamide and 1,8-dichloro-3-(5-(difluoromethyl)-1,3,4-thiadiazol-2-yl)-N-(1-methylcyclopropyl)imidazo[1,5-a]pyridine-6-sulfonamide

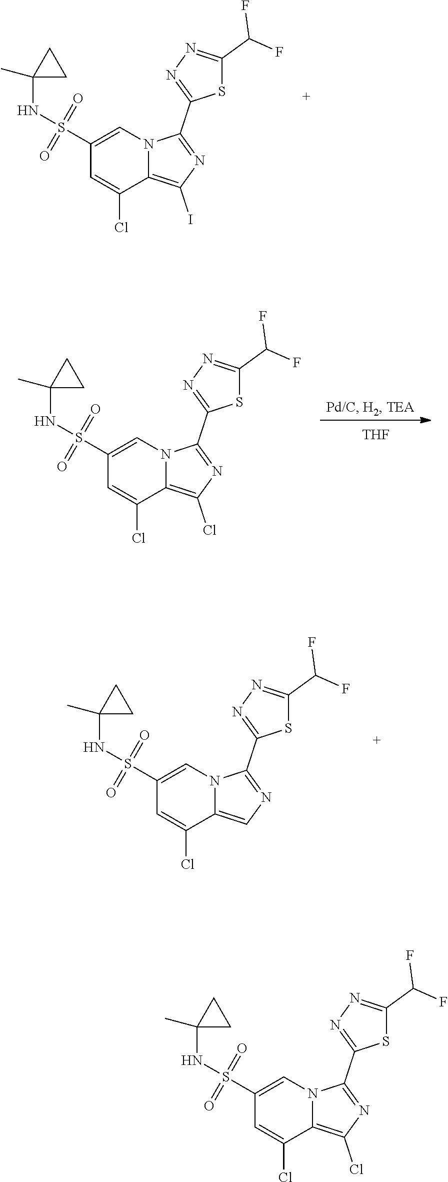 [SMILES: CC1(NS(=O)(=O)c2cc(Cl)c3c(I)nc(-c4nnc(C(F)F)s4)n3c2)CC1 ; CC1(NS(=O)(=O)c2cc(Cl)c3c(Cl)nc(-c4nnc(C(F)F)s4)n3c2)CC1 ; "Pd/C, H2, TEA" ; CC1(NS(=O)(=O)c2cc(Cl)c3cnc(-c4nnc(C(F)F)s4)n3c2)CC1 ; "THF" ; CC1(NS(=O)(=O)c2cc(Cl)c3c(Cl)nc(-c4nnc(C(F)F)s4)n3c2)CC1]

To a mixture of 8-chloro-3-(5-(difluoromethyl)-1,3,4-thiadiazol-2-yl)-1-iodo-N-(1-methylcyclopropyl)imidazo[1,5-a]pyridine-6-sulfonamide and 1,8-dichloro-3-(5-(difluoromethyl)-1,3,4-thiadiazol-2-yl)-N-(1-methylcyclopropyl)imidazo[1,5-a]pyridine-6-sulfonamide (430 mg, crude) in THF (5 mL) were added TEA (239.18 mg, 2.36 mmol, 329.00 μL) and Pd/C (787.90 μmol, 10% purity). The reaction mixture was de-gassed with H₂ (balloon, 15 Psi) three times, then stirred at 25° C. under H₂ atmosphere (balloon, 15 Psi) for 3 h and filtered. The filtrate was concentrated under vacuum to give a residue which was purified by preparative HPLC (column: Phenomenex luna C18 150*25 mm*10 μm; mobile phase: A: 0.225% formic acid in water, B: MeCN; B %: 55%-70%, 10 min) and lyophilized directly to give the product 8-chloro-3-(5-(difluoromethyl)-1,3,4-thiadiazol-2-yl)-N-(1-methylcyclopropyl)imidazo[1,5-a]pyridine-6-sulfonamide (80 mg, 175.30 μmol, 92% purity) as a yellow solid and the product 1,8-dichloro-3-(5-(difluoromethyl)-1,3,4-thiadiazol-2-yl)-N-(1-methylcyclopropyl)imidazo[1,5-a]pyridine-6-sulfonamide (120 mg) as a yellow solid.

### 8-chloro-3-(5-(difluoromethyl)-1,3,4-thiadiazol-2-yl)-N-(1-methylcyclopropyl)imidazo[1,5-a]pyridine-6-sulfonamide

RT 0.476 min (method 3); m/z 420.0 (M+H)⁺ (ESI⁺); ¹H NMR (CDCl₃, 400 MHz): 10.11 (s, 1H), 7.91 (s, 1H), 7.38 (s, 1H), 7.09 (t, J=53.6 Hz, 1H), 5.16 (s, 1H), 1.41 (s, 3H), 0.93-0.90 (m, 2H), 0.65-0.62 (m, 2H)

### 1,8-dichloro-3-(5-(difluoromethyl)-1,3,4-thiadiazol-2-yl)-N-(1-methylcyclopropyl)imidazo[1,5-a]pyridine-6-sulfonamide

RT 0.509 min (method 1); m/z 454.0 (M+H)⁺ (ESI⁺); ¹H NMR (CDCl₃, 400 MHz) 10.10 (s, 1H), 7.35 (s, 1H), 7.09 (t, J=53.6 Hz, 1H), 5.17 (s, 1H), 1.41 (s, 3H), 0.96-0.87 (m, 2H), 0.68-0.60 (m, 2H)

### Preparation of Example 15

### N-(1-cyanocyclopropyl)-3-(5-(difluoromethyl)-1,3,4-thiadiazol-2-yl)-8-(4-(methoxymethyl)piperidin-yl)imidazo[1,5-a]pyridine-6-sulfonamide

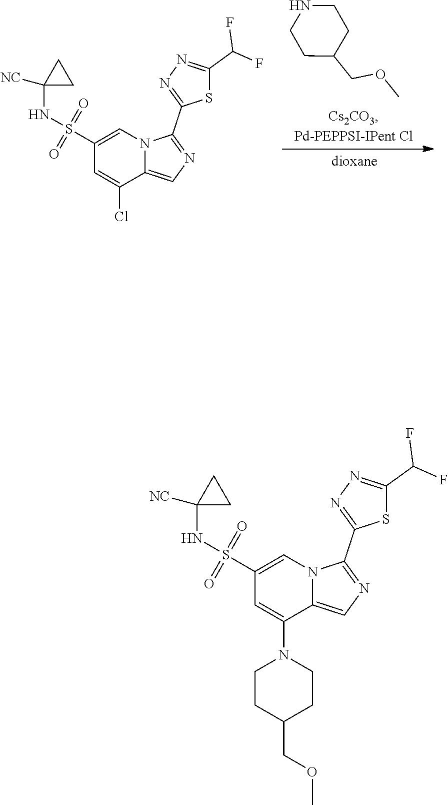

To a solution of 8-chloro-N-(1-cyanocyclopropyl)-3-(5-(difluoromethyl)-1,3,4-thiadiazol-2-yl)imidazo[1,5-a]pyridine-6-sulfonamide (30 mg, 69.63 μmol) and 4-(methoxymethyl)piperidine (17.99 mg, 139.26 μmol) in dioxane (1 mL) were added Cs₂CO₃ (68.06 mg, 208.89 μmol) and Pd-PEPPSI-IPent Cl (6.77 mg, 6.96 μmol) under a nitrogen atmosphere. The reaction mixture was stirred at 100° C. for 12 h, then, filtered and the filtrate was concentrated under vacuum. The resulting residue was purified by preparative TLC (EtOAc: petroleum ether=1:1) to give an unpure product (22 mg)which was further purified by preparative HPLC (column: Unisil 3-100 C18 Ultra 150*50 mm*3 μm; mobile phase: A: 0.225% formic acid in water, B: MeCN; B %: 43%-73%, 10 min) and lyophilized directly to give the product N-(1-cyanocyclopropyl)-3-(5-(difluoromethyl)-1,3,4-thiadiazol-2-yl)-8-(4-(methoxymethyl)piperidin-1-yl)imidazo[1,5-a]pyridine-6-sulfonamide (9.02 mg, 17.06 μmol, 24.49% yield, 99% purity) as a light yellow solid.

RT 0.458 min (method 3); m/z 524.0 (M+H)⁺ (ESI⁺); ¹H NMR (CD₃OD, 400 MHz): 9.87 (s, 1H), 7.84 (s, 1H), 7.33 (t, J=53.6 Hz, 1H), 6.74 (d, J=1.2 Hz, 1H), 3.87 (d, J=12.4 Hz, 2H), 3.37 (s, 3H), 3.36 (s, 2H), 3.02-2.93 (m, 2H), 1.99-1.91 (m, 2H), 1.91-1.81 (m, 1H), 1.64-1.55 (m, 2H), 1.55-1.49 (m, 2H), 1.49-1.42 (m, 2H).

### Preparation of Example 16

### N-(1-cyanocyclopropyl)-3-(5-(difluoromethyl)-1,3,4-thiadiazol-2-yl)-8-(4-methoxypiperidin-1-yl)imidazo[1,5-a]pyridine-6-sulfonamide

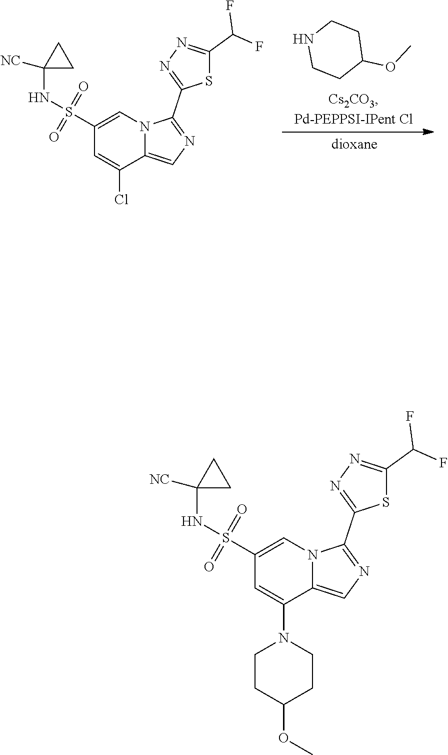

To a solution of 8-chloro-N-(1-cyanocyclopropyl)-3-(5-(difluoromethyl)-1,3,4-thiadiazol-2-yl) imidazo [1,5-a]pyridine-6-sulfonamide (30 mg, 69.63 μmol) and 4-methoxypiperidine (16.04 mg, 139.26 μmol) in dioxane (1 mL) were added Cs₂CO₃ (68.06 mg, 208.89 μmol) and Pd-PEPPSI-IPent Cl (6.77 mg, 6.96 μmol). The mixture was degassed with N₂ three times, then stirred at 100° C. for 12 h, cooled down to 20° C. and filtered. The filtrate was concentrated under vacuum to give a residue which was purified by preparative HPLC (column: Phenomenex luna C18 150*25 mm*10 μm; mobile phase: A: 0.225% formic acid in water, B: MeCN; B %: 34%-64%, 10.5 min) and lyophilized directly to give the product N-(1-cyanocyclopropyl)-3-(5-(difluoromethyl)-1,3,4-thiadiazol-2-yl)-8-(4-methoxy-1-piperidyl) imidazo[1,5-a]pyridine-6-sulfonamide (11 mg, 21.59 μmol, 31.00% yield, 100% purity) as a yellow solid.

RT 0.434 min (method 3); m/z 510.1 (M+H)⁺ (ESI⁺); ¹H NMR (CDCl₃, 400 MHz): 9.94 (s, 1H), 7.74 (s, 1H), 7.08 (t, J=54.0 Hz, 1H), 6.67 (s, 1H), 5.92-5.61 (br, 1H), 3.64-3.61 (m, 2H), 3.55-3.47 (m, 1H), 3.42 (s, 3H), 3.25-3.14 (m, 2H), 2.15-2.06 (m, 2H), 1.92-1.81 (m, 2H), 1.73-1.67 (m, 2H), 1.52-1.49 (m, 2H).

### Preparation of Example 17

### N-(1-cyanocyclopropyl)-3-(5-(difluoromethyl)-1,3,4-thiadiazol-2-yl)-8-(4-(hydroxymethyl)piperidin-1-yl)imidazo[1,5-a]pyridine-6-sulfonamide

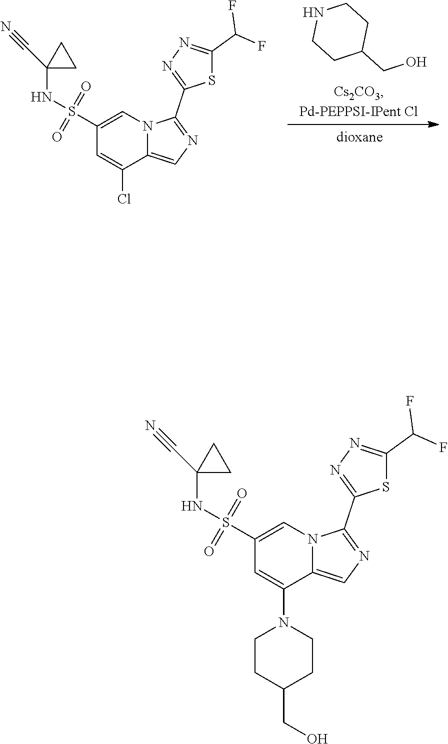 [SMILES: N#CC1(NS(=O)(=O)c2cc(Cl)c3cnc(-c4nnc(C(F)F)s4)n3c2)CC1 ; OCC1CCNCC1 ; "dioxane" ; N#CC1(NS(=O)(=O)c2cc(N3CCC(CO)CC3)c3cnc(-c4nnc(C(F)F)s4)n3c2)CC1]

To a solution of 8-chloro-N-(1-cyanocyclopropyl)-3-(5-(difluoromethyl)-1,3,4-thiadiazol-2-yl)imidazo[1,5-a]pyridine-6-sulfonamide (30.00 mg, 69.63 μmol) and 4-piperidylmethanol (16.04 mg, 139.26 μmol) in dioxane (1 mL) were added Cs₂CO₃ (68.06 mg, 208.89 μmol) and Pd-PEPPSI-IPent Cl (6.77 mg, 6.96 μmol). The mixture was degassed with N₂ three times, then stirred at 80° C. for 12 h, cooled down to 20° C. and filtered. The filtrate was concentrated under vacuum. The resulting residue purified by preparative TLC (petroleum ether:EtOAc=1:1) to give an unpure product which was then further purified by preparative HPLC (column: Phenomenex luna C18 150*25 mm*10 μm; mobile phase: A: 0.225% formic acid in water, B: MeCN; B %: 33%-63%, 10 min) and lyophilized directly to give the product N-(1-cyanocyclopropyl)-3-(5-(difluoromethyl)-1,3,4-thiadiazol-2-yl)-8-(4-(hydroxymethyl)-1-piperidyl)imidazo[1,5-a]pyridine-6-sulfonamide (5.8 mg, 10.81 μmol, 15.53% yield, 95.00% purity) as a yellow solid.

RT 0.404 min (method 3); m/z 510.0 (M+H)⁺ (ESI⁺); ¹H NMR (CDCl₃, 400 MHz): 9.95 (s, 1H), 7.76 (s, 1H), 7.09 (t, J=54.0 Hz, 1H), 6.68 (d, J=1.4 Hz, 1H), 5.62 (s, 1H), 3.89 (d, J=12.4 Hz, 2H), 3.65 (d, J=6.2 Hz, 2H), 3.05-2.92 (td, J=12.0, 2.4 Hz, 2H), 1.97 (dd, J=12.8, 2.4 Hz, 2H), 1.88-1.78 (m, 1H), 1.75-1.70 (m, 2H), 1.68-1.63 (m, 1H), 1.63-1.60 (m, 2H), 1.56-1.52 (m, 2H).

### Preparation of Example 18

### 3-(5-(difluoromethyl)-1,3,4-thiadiazol-2-yl)-8-(4-(hydroxymethyl)piperidin-1-yl)-N-(1-methylcyclopropyl)imidazo[1,5-a]pyridine-6-sulfonamide

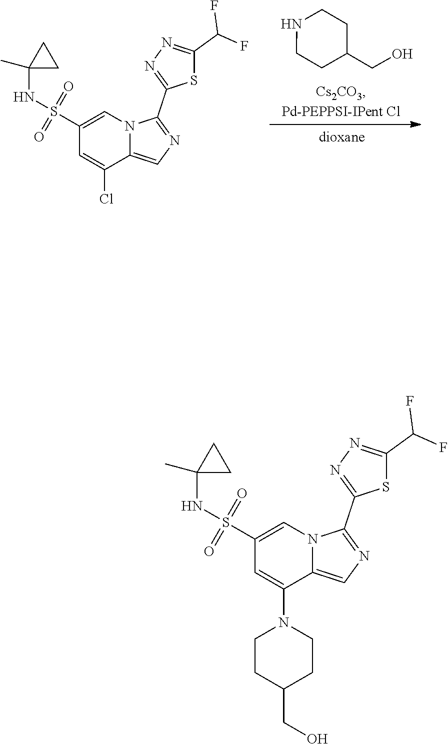 [SMILES: CC1(NS(=O)(=O)c2cc(Cl)c3cnc(-c4nnc(C(F)F)s4)n3c2)CC1 ; OCC1CCNCC1 ; "dioxane" ; CC1(NS(=O)(=O)c2cc(N3CCC(CO)CC3)c3cnc(-c4nnc(C(F)F)s4)n3c2)CC1]

To a solution of 8-chloro-3-(5-(difluoromethyl)-1,3,4-thiadiazol-2-yl)-N-(1-methylcyclopropyl)imidazo[1,5-a]pyridine-6-sulfonamide (10 mg, 23.82 μmol) and 4-piperidylmethanol (4.12 mg, 35.73 μmol) in dioxane (1 mL) were added Cs₂CO₃ (23.28 mg, 71.46 μmol) and Pd-PEPPSI-IPent Cl (2.32 mg, 2.38 μmol) The mixture was degassed with N₂ three times, then stirred at 100° C. for 30 min, cooled down to 20° C. and filtered. The filtrate was concentrated under vacuum to give a residue which was purified by preparative HPLC (column: Unisil 3-100 C18 Ultra 150*50 mm*3 μm; mobile phase: A: 0.225% formic acid in water, B: MeCN; B %: 35%-65%, 10 min) and lyophilized directly to give the product 3-(5-(difluoromethyl)-1,3,4-thiadiazol-2-yl)-8-(4-(hydroxymethyl)-1-piperidyl)-N-(1-methylcyclopropyl)imidazo[1,5-a]pyridine-6-sulfonamide (6.4 mg, 11.94 μmol, 50.12% yield, 93% purity) as a yellow solid.

RT 0.450 min (method 3); m/z 499.0 (M+H)⁺ (ESI⁺); ¹H NMR (CDCl₃, 400 MHz): 9.83 (s, 1H), 7.71 (s, 1H), 7.08 (t, J=54.4 Hz, 1H), 6.64 (s, 1H), 5.01 (s, 1H), 3.82 (d, J=12.4 Hz, 2H), 3.64 (d, J=6.0 Hz, 2H), 2.93 (t, J=12.0 Hz, 2H), 2.04-1.96 (m, 2H), 1.81-1.76 (m, 1H), 1.65-1.58 (m, 2H), 1.39 (s, 3H), 0.95 (s, 2H), 0.64-0.55 (m, 2H).

### Preparation of Example 19

### 3-(5-(difluoromethyl)-1,3,4-thiadiazol-2-yl)-8-(4-(2-hydroxypropan-2-yl)piperidin-1-yl)-N-(1-methylcyclopropyl)imidazo[1,5-a]pyridine-6-sulfonamide

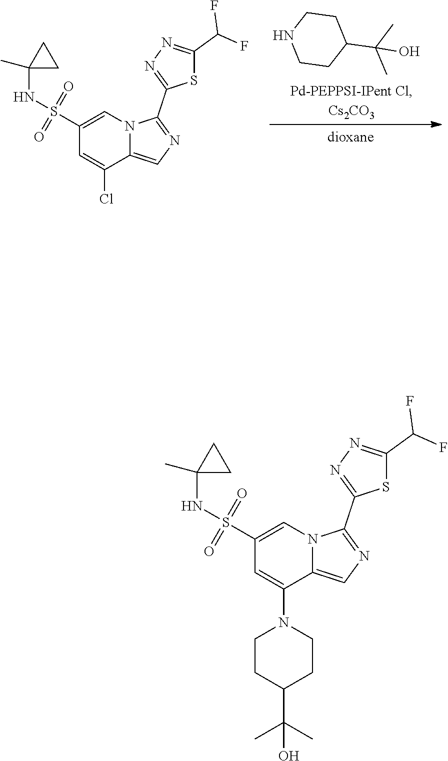

To a solution of 8-chloro-3-(5-(difluoromethyl)-1,3,4-thiadiazol-2-yl)-N-(1-methylcyclopropyl)imidazo [1,5-a]pyridine-6-sulfonamide (10 mg, 23.82 μmol) and 2-(4-piperidyl)propan-2-ol (5.12 mg, 35.73 μmol) in dioxane (1 mL) were added Cs₂CO₃ (23.28 mg, 71.45 μmol) and Pd-PEPPSI-IPent Cl (2.32 mg, 2.38 μmol). The mixture was degassed with N₂ three times, then stirred at 100° C. for 1 h, cooled down to 20° C. and filtered. The filtrate was concentrated under vacuum to give a residue which was purified by preparative HPLC (column: Phenomenex luna C18 150*25 mm*10 μm; mobile phase: A: 0.225% formic acid in water, B: MeCN; 45%-75%, 10 min) and lyophilized directly to give the product 3-(5-(difluoromethyl)-1,3,4-thiadiazol-2-yl)-8-(4-(1-hydroxy-1-methyl-ethyl)-1-piperidyl)-N-(1-methylcyclopropyl)imidazo[1,5-a]pyridine-6-sulfonamide (1 mg, 1.71 μmol, 7.16% yield, 97.7% purity, FA salt) as a yellow solid.

RT 0.474 min (method 3); m/z 527.0 (M+H)⁺ (ESI⁺); ¹H NMR (DMSO-d₆, 400 MHz): 9.56 (s, 1H), 8.49-8.39 (br, 1H), 8.38-8.33 (br, 1H), 7.94 (s, 1H), 7.66 (t, J=52.4 Hz, 1H), 6.66 (s, 1H), 4.36-4.04 (m, 1H), 3.84 (d, J=12.2 Hz, 2H), 2.83 (t, J=11.2 Hz, 2H), 1.90-1.83 (m, 2H), 1.58-1.49 (m, 2H), 1.49 (s, 1H), 1.15 (s, 3H), 1.10 (s, 6H), 0.72 (s, 2H), 0.44 (s, 2H)

### Preparation of Example 20

### 3-(5-(difluoromethyl)-1,3,4-thiadiazol-2-yl)-8-(4-(methoxymethyl)piperidin-1-yl)-N-(1-methylcyclopropyl)imidazo[1,5-a]pyridine-6-sulfonamide

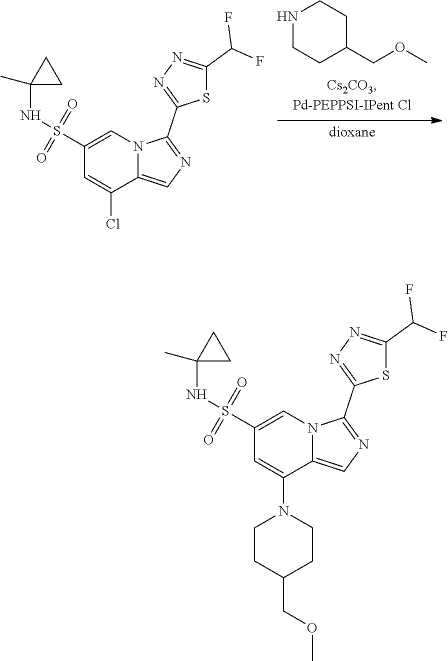

To a solution of 8-chloro-3-(5-(difluoromethyl)-1,3,4-thiadiazol-2-yl)-N-(1-methylcyclopropyl)imidazo[1,5-a]pyridine-6-sulfonamide (10 mg, 23.82 μmol) and 4-(methoxymethyl)piperidine (4.62 mg, 35.73 μmol) in dioxane (1 mL) were added Cs₂CO₃ (23.28 mg, 71.45 μmol) and Pd-PEPPSI-IPent Cl (2.32 mg, 2.38 μmol). The mixture was degassed with N₂ three times, then stirred at 100° C. for 30 min, and filtered. The filtrate was concentrated under vacuum to give a residue, which was purified by preparative HPLC (column: Unisil 3-100 C18 Ultra 150*50 mm*3 um; mobile phase: A: 0.225% formic acid in water, B: MeCN; B %: 35%-65%, 10 min) and lyophilized directly to give the product 3-(5-(difluoromethyl)-1,3,4-thiadiazol-2-yl)-8-(4-(methoxymethyl)-1-piperidyl)-N-(1-methylcyclopropyl)imidazo[1,5-a]pyridine-6-sulfonamide (4.73 mg, 9.02 μmol, 37.85% yield, 97.7% purity) as a yellow solid.

RT 0.499 min (method 3); m/z 513.2 (M+H)⁺ (ESI⁺); ¹H NMR (CDCl₃ 400 MHz): 9.83 (s, 1H), 7.70 (s, 1H), 7.08 (t, J=54.0 Hz, 1H), 6.63 (d, J=1.2 Hz, 1H), 5.02 (s, 1H), 3.84-3.74 (m, 2H), 3.40 (s, 3H), 3.35 (d, J=6.0 Hz, 2H), 2.98-2.86 (m, 2H), 1.99-1.92 (m, 2H), 1.88 (s, 1H), 1.59-1.58 (m, 2H), 1.38 (s, 3H), 0.97-0.91 (m, 2H), 0.63-0.57 (m, 2H).

### Preparation of Example 21

### 1-chloro-3-(5-(difluoromethyl)-1,3,4-thiadiazol-2-yl)-8-(4-(methoxymethyl)piperidin-1-yl)-N-(1-methylcyclopropyl)imidazo[1,5-a]pyridine-6-sulfonamide

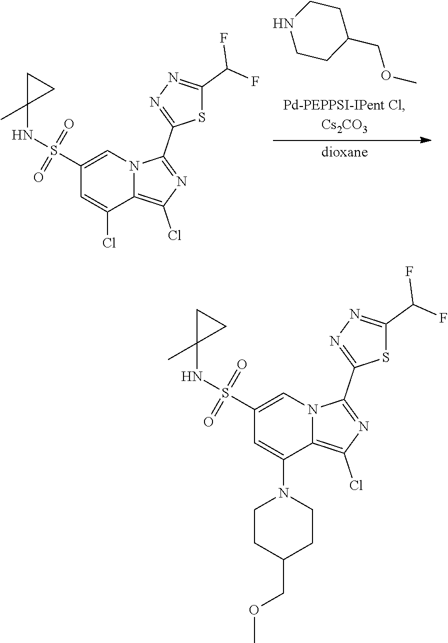

To a solution of 1,8-dichloro-3-(5-(difluoromethyl)-1,3,4-thiadiazol-2-yl)-N-(1-methylcyclopropyl)imidazo[1,5-a]pyridine-6-sulfonamide (30 mg, 66.04 μmol) and 4-(methoxymethyl)piperidine (12.80 mg, 99.05 μmol) in dioxane (1 mL) were added Cs₂CO₃ (64.55 mg, 198.11 μmol) and Pd-PEPPSI-IPent Cl (6.42 mg, 6.60 μmol). The mixture was degassed with N₂ three times, then stirred at 100° C. for 30 min, cooled down to 20° C. and filtered. The filtrate was concentrated under vacuum. The resulting residue was purified by preparative TLC (petroleum ether:EtOAc=0:1) to give an impure product which was further purified by preparative HPLC (column: Phenomenex luna C18 150*25 mm*10 μm; mobile phase: A: 0.225% formic acid in water, B: MeCN; B %: 60%-90%, 10 min) and lyophilized to give the product 1-chloro-3-(5-(difluoromethyl)-1,3,4-thiadiazol-2-yl)-8-(4-(methoxymethyl)-1-piperidyl)-N-(1-methylcyclopropyl)imidazo[1,5-a]pyridine-6-sulfonamide (5.6 mg, 9.44 μmol, 14.30% yield, 100% purity, FA salt) as a yellow solid.

RT 0.543 min (method 3); m/z 547.0 (M+H)⁺ (ESI⁺); ¹H NMR (DMSO-d₆, 400 MHz): 9.59 (d, J=0.8 Hz, 1H), 8.50 (s, 1H), 8.37 (s, 1H), 7.68 (t, J=53.2 Hz, 1H), 6.80 (d, J=0.8 Hz, 1H), 3.46 (s, 2H), 3.28 (s, 2H), 3.27 (s, 3H), 2.73 (t, J=11.4 Hz, 2H), 1.88-1.81 (m, 2H), 1.80-1.72 (m, 1H), 1.58-1.46 (m, 2H), 1.16 (s, 3H), 0.77-0.69 (m, 2H), 0.49-0.42 (m, 2H).

### Preparation of Example 22

### 3-(5-(difluoromethyl)-1,3,4-thiadiazol-2-yl)-8-(4-(1-hydroxyethyl)piperidin-1-yl)-N-(1-methylcyclopropyl)imidazo[1,5-a]pyridine-6-sulfonamide

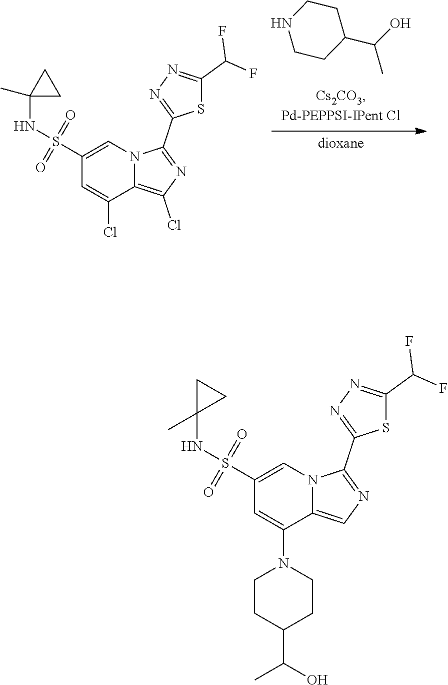

To a solution of 1,8-dichloro-3-(5-(difluoromethyl)-1,3,4-thiadiazol-2-yl)-N-(1-methylcyclopropyl) imidazo[1,5-a]pyridine-6-sulfonamide (20 mg, 44.02 μmol) and 1-(4-piperidyl)ethanol (22.75 mg, 176.08 μmol) in dioxane (1 mL) were added Cs₂CO₃ (28.69 mg, 88.04 μmol) and Pd-PEPPSI-IPent Cl (4.28 mg, 4.40 μmol) under a nitrogen atmosphere. The mixture was stirred at 100° C. for 16 hr, then cooled down to 20° C. and filtered. The filtrate was concentrated under vacuum. The resulting residue was purified by preparative TLC (EtOAc:Petroleum ether=3:1) to give an unpure product which was further purified by preparative HPLC (column: Phenomenex luna C18 150*25 mm*10 μm; mobile phase: A: 0.225% formic acid in water, B: MeCN; B %: 43%-73%, 10 min) and lyophilized directly to give the product 3-(5-(difluoromethyl)-1,3,4-thiadiazol-2-yl)-8-(4-(1-hydroxyethyl)piperidin-1-yl)-N-(1-methylcyclopropyl)imidazo[1,5-a]pyridine-6-sulfonamide (1.4 mg, 2.59 μmol, 5.89% yield, 95.82% purity) as a yellow solid.

RT 0.472 min (method 3); m/z 513.0 (M+H)⁺ (ESI⁺); ¹H NMR (CD₃OD, 400 MHz): 9.79 (s, 1H), 7.83 (s, 1H), 7.33 (t, J=53.6 Hz, 1H), 6.70 (s, 1H), 3.87 (d, J=12.4 Hz, 2H), 3.61 (t, J=6.0 Hz, 1H), 2.96-2.84 (m, 2H), 2.06 (d, J=10.8 Hz, 1H), 1.85 (d, J=12.8 Hz, 1H), 1.66-1.60 (m, 1H), 1.59-1.48 (m, 2H), 1.26 (s, 3H), 1.23 (d, J=6.4 Hz, 3H), 0.86-0.81 (m, 2H), 0.53-0.48 (m, 2H).

### Preparation of Example 23

### Chloro-3-(5-(difluoromethyl)-1,3,4-thiadiazol-2-yl)-8-(4-(1-hydroxyethyl)piperidin-1-yl)-N-(1-methylcyclopropyl)imidazo[1,5-a]pyridine-6-sulfonamide

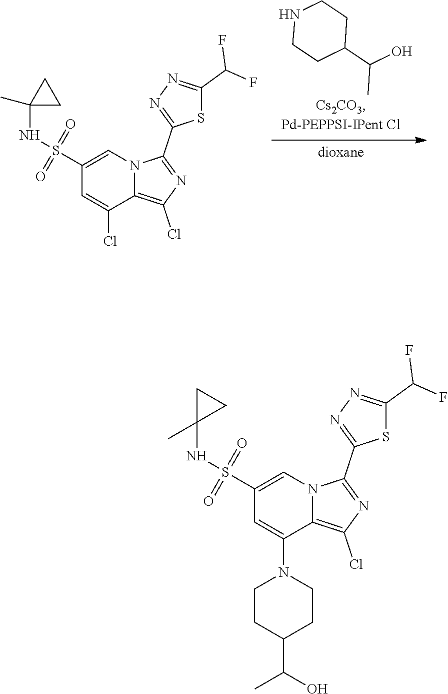

To a solution of 1,8-dichloro-3-(5-(difluoromethyl)-1,3,4-thiadiazol-2-yl)-N-(1-methylcyclopropyl)imidazo[1,5-a]pyridine-6-sulfonamide (35 mg, 77.04 μmol) and 1-(4-piperidyl)ethanol (19.91 mg, 154.08 μmol) in dioxane (1 mL) were added Pd-PEPPSI-IPent Cl (7.49 mg, 7.70 μmol) and Cs₂CO₃ (75.30 mg, 231.12 μmol) under a nitrogen atmosphere. The mixture was stirred at 100° C. for 20 min, then cooled down to 20° C. and filtered. The filtrate was concentrated under vacuum to give a residue which was purified by preparative HPLC (column: Phenomenex luna C18 150*25 mm*10 μm; mobile phase: A: 0.225% formic acid in water, B: MeCN; B %: 50%-80%, 10 min) and lyophilized directly to give the product 1-chloro-3-(5-(difluoromethyl)-1,3,4-thiadiazol-2-yl)-8-(4-(1-hydroxyethyl)piperidin-1-yl)-N-(1-methylcyclopropyl)imidazo[1,5-a]pyridine-6-sulfonamide (2.9 mg, 5.14 μmol, 6.67% yield, 97.16% purity) as a yellow solid.

RT 0.493 min (method 3); m/z 547.0 (M+H)⁺ (ESI⁺); ¹H NMR (DMSO-d₆, 400 MHz): 9.59 (s, 1H), 8.49 (s, 1H), 7.68 (t, J=52.8 Hz, 1H), 6.79 (s, 1H), 4.45 (d, J=4.4 Hz, 1H), 3.49 (d, J=11.2 Hz, 2H), 3.47-3.42 (m, 1H), 2.69 (t, J=11.2 Hz, 1H), 1.96 (d, J=12.0 Hz, 1H), 1.72 (d, J=11.6 Hz, 1H), 1.63-1.47 (m, 2H), 1.41-1.32 (m, 1H), 1.17 (s, 3H), 1.10 (d, J=6.2 Hz, 3H), 0.76-0.70 (m, 2H), 0.49-0.43 (m, 2H).

### Preparation of Example 24

### 1-chloro-3-(5-(difluoromethyl)-1,3,4-thiadiazol-2-yl)-8-(4-(2-hydroxypropan-2-yl)piperidin-1-yl)-N-(1-methylcyclopropyl)imidazo[1,5-a]pyridine-6-sulfonamide

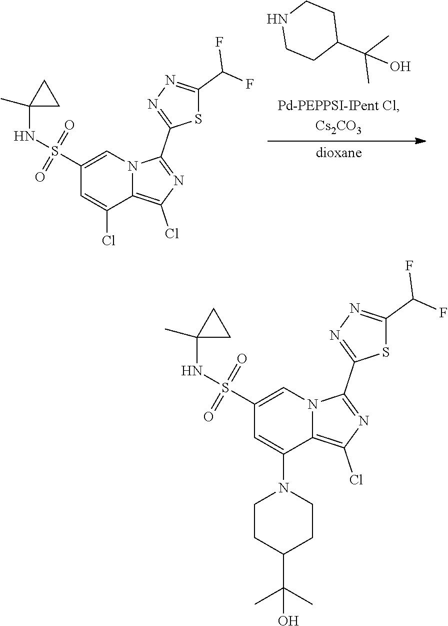

To a solution of 1,8-dichloro-3-(5-(difluoromethyl)-1,3,4-thiadiazol-2-yl)-N-(1-methylcyclopropyl) imidazo[1,5-a]pyridine-6-sulfonamide (20 mg, 44.02 μmol) and 2-(4-piperidyl)propan-2-ol (9.46 mg, 66.04 μmol) in dioxane (1 mL) were added Cs₂CO₃ (43.03 mg, 132.07 μmol) and Pd-PEPPSI-IPent Cl (4.28 mg, 4.40 μmol). The mixture was degassed with N₂ three times, then stirred at 100° C. for 30 min, cooled down to 20° C. and filtered. The filtrate was concentrated undervacuum. The resulting residue was purified by preparative TLC (EtOAc:Petroleum ether=1:0) to give an impure product which was further purified by preparative HPLC (column: Phenomenex luna C18 150*25 mm*10 μm; mobile phase: A: 0.225% formic acid in water, B: MeCN; B %: 50%-80%, 10 min) and lyophilized directly to give the product 1-chloro-3-(5-(difluoromethyl)-1,3,4-thiadiazol-2-yl)-8-(4-(1-hydroxy-1-methyl-ethyl)-1-piperidyl)-N-(1-methylcyclopropyl)imidazo[1,5-a]pyridine-6-sulfonamide (3.91 mg, 6.18 μmol, 14.03% yield, 95.9% purity, FA salt) as a yellow solid.

RT 0.507 min (method 3); m/z 561.0 (M+H)⁺ (ESI⁺); ¹H NMR (DMSO-d₆, 400 MHz): 9.59 (s, 1H), 8.52 (s, 1H), 8.39 (s, 1H), 7.07 (t, J=53.2 Hz, 1H), 6.79 (s, 1H), 4.41-4.02 (m, 1H), 3.52 (d, J=11.4 Hz, 2H), 2.67 (t, J=11.4 Hz, 2H), 1.86 (d, J=12.0 Hz, 2H), 1.67-1.51 (m, 2H), 1.47-1.36 (m, 1H), 1.17 (s, 3H), 1.11 (s, 6H), 0.79-0.68 (m, 2H), 0.52-0.40 (m, 2H)

### Preparation of Example 25a

### 1,8-dichloro-3-(5-(difluoromethyl)-1,3,4-thiadiazol-2-yl)-N-(1-(fluoromethyl)cyclopropyl)imidazo[1,5-a]pyridine-6-sulfonamide

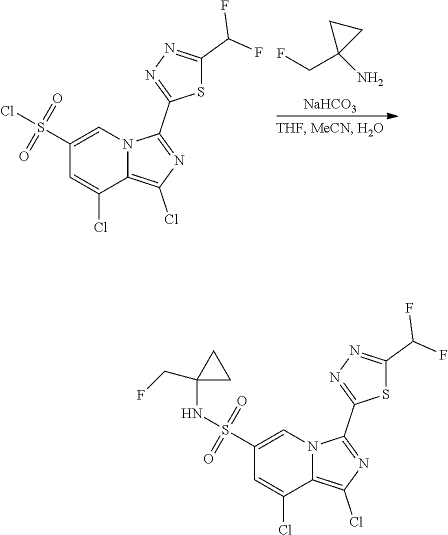

To a mixture of 1-(fluoromethyl)cyclopropanamine (179.54 mg, 1.43 mmol, HCl salt) in water (3 mL) and THE (3 mL) were added at 0° C. NaHCO₃ (240.23 mg, 2.86 mmol, 111.22 μL) and then, a solution of 1,8-dichloro-3-[5-(difluoromethyl)-1,3,4-thiadiazol-2-yl]imidazo[1,5-a]pyridine-6-sulfonyl chloride (400 mg, 953.19 μmol) in THE (3 mL) dropwise. The resulting mixture was stirred at 0° C. for 20 min, quenched with H₂O (25 mL) and extracted with EtOAc (30 mL; 2×). The combined organic layer was washed with brine (50 mL; 2×), dried over Na₂SO₄, filtered and the filtrate was concentrated under vacuum to give a residue, which was purified by flash silica gel chromatography (ISCO®; 20 g SepaFlash® Silica Flash Column, Eluent of 0˜50% EtOAc/Petroleum ether; gradient @30 mL/min) to give the product 1,8-dichloro-3-[5-(difluoromethyl)-1,3,4-thiadiazol-2-yl]-N-[1-(fluoromethyl)cyclopropyl]imidazo[1,5-a]pyridine-6-sulfonamide (180 mg, 343.01 μmol, 35.99% yield, 90% purity) as a yellow solid.

RT 0.52 min (method 1); m/z 471.8&173.8 (M+H)⁺ (ESI⁺); ¹H NMR (DMSO-d₆, 400 MHz): 9.80 (s, 1H), 9.03 (s, 1H), 7.71 (t, J=53.2 Hz, 1H), 7.48 (s, 1H), 4.25 (d, J=52.8 Hz, 2H), 0.91-0.89 (m, 2H), 0.83-0.77 (m, 2H).

### Preparation of Example 25

### 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide

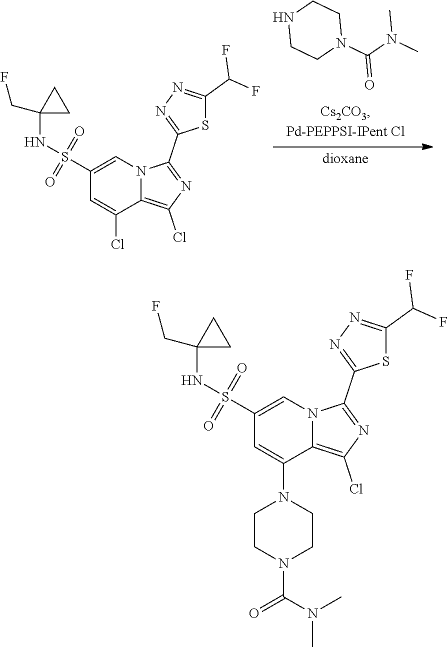 [SMILES: O=S(=O)(NC1(CF)CC1)c1cc(Cl)c2c(Cl)nc(-c3nnc(C(F)F)s3)n2c1 ; CN(C)C(=O)N1CCNCC1 ; CN(C)C(=O)N1CCN(c2cc(S(=O)(=O)NC3(CF)CC3)cn3c(-c4nnc(C(F)F)s4)nc(Cl)c23)CC1]

To a mixture of 1,8-dichloro-3-[5-(difluoromethyl)-1,3,4-thiadiazol-2-yl]-N-[1-(fluoromethyl)cyclopropyl]imidazo[1,5-a]pyridine-6-sulfonamide (50 mg, 105.87 μmol) in dioxane (2 mL) were added Cs₂CO₃ (68.99 mg, 211.73 μmol), N,N-dimethylpiperazine-1-carboxamide (33.29 mg, 211.73 μmol) and Pd-PEPPSI-IPent Cl (9.11 mg, 10.59 μmol). The reaction mixture was degassed with N₂ three times, stirred at 100° C. for 1 h and filtered. The filtrate was concentrated under vacuum to give a residue, which was purified by preparative HPLC (column: Phenomenex luna C18 150*25 mm*10 μm; mobile phase: A: 0.221% NH₃·H₂O in water, B: MeCN; B %: 43%-73%, 10 min) and lyophilized directly to give the product 4-[1-chloro-3-[5-(difluoromethyl)-1,3,4-thiadiazol-2-yl]-6-[[1-(fluoromethyl)cyclopropyl]sulfamoyl]imidazo[1,5-a]pyridin-8-yl]-N,N-dimethyl-piperazine-1-carboxamide (12.4 mg, 20.49 μmol, 19.36% yield, 98% purity) as a yellow solid.

RT 0.494 min (method 2); m/z 593.0 (M+H)⁺ (ESI⁺); ¹H NMR (CDCl₃, 400 MHz): 9.90 (s, 1H), 7.07 (t, J=54.0 Hz, 1H), 6.73 (s, 1H), 5.46 (s, 1H), 4.27 (d, J=48.4 Hz, 2H), 3.55-3.51 (m, 4H), 3.22-3.16 (m, 4H), 2.90 (s, 6H), 1.15-1.14 (m, 2H), 0.89-0.87 (m, 2H)

### Preparation of Example 26

### tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-3,6-dihydropyridine-1(2H)-carboxylate

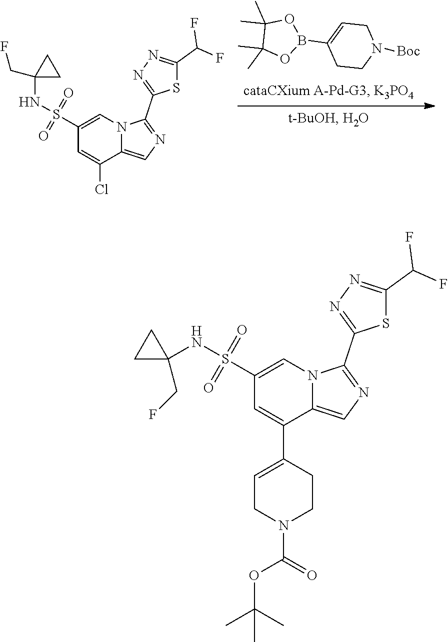

To a mixture of 8-chloro-3-(5-(difluoromethyl)-1,3,4-thiadiazol-2-yl)-N-(1-(fluoromethyl)cyclopropyl)imidazo[1,5-a]pyridine-6-sulfonamide (80 mg, 182.71 μmol) in t-BuOH (2 mL) and H₂O (0.2 mL) was added tert-butyl 4-(4,4,5,5-tetramethyl-1,3,2-dioxaborolan-2-yl)-5,6-dihydropyridine-1(2H)-carboxylate (169.49 mg, 548.14 μmol) and K₃PO₄ (46.54 mg, 219.25 μmol). The mixture was degassed and purged with N₂ (3×), then cataCXium A-Pd-G3 (13.31 mg, 18.27 μmol) was added. The mixture was stirred at 60° C. for 16 h under N₂ atmosphere. The reaction mixture was extracted with EtOAc (10 mL, 3×). The combined organic layer was washed with brine (30 mL) and concentrated under vacuum. The residue was purified by preparative TLC (Petroleum ether:Ethyl acetate=2/1) and preparative HPLC (column: Phenomenex Synergi C18 150*25 mm*10 μm; mobile phase: A: 0.225% formic acid in water, B: MeCN; B %: 40%-70%, 10 min) and lyophilized directly to give the product tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-3,6-dihydropyridine-1(2H)-carboxylate (23.85 mg, 37.12 μmol, 20.32% yield, 94% purity) as a yellow solid.

RT 0.516 min (method 3); m/z 585.3 (M+H)⁺ (ESI⁺); ¹H NMR (CDCl₃, 400 MHz): 10.14 (s, 1H), 7.86 (s, 1H), 7.20 (s, 1H), 7.09 (t, J=56.0 Hz, 1H), 6.36 (s, 1H), 5.49 (s, 1H), 4.29 (d, J=48.0 Hz, 2H), 4.20-4.18 (m, 2H), 3.73 (t, J=8.0 Hz, 2H), 2.63-2.57 (m, 2H), 1.53 (s, 9H), 1.18-1.13 (m, 2H), 0.91-0.88 (m, 2H).

### Preparation of Example 27

### 3-(5-(difluoromethyl)-1,3,4-thiadiazol-2-yl)-N-(1-(fluoromethyl)cyclopropyl)-8-(1,2,3,6-tetrahydropyridin-4-yl)imidazo[1,5-a]pyridine-6-sulfonamide

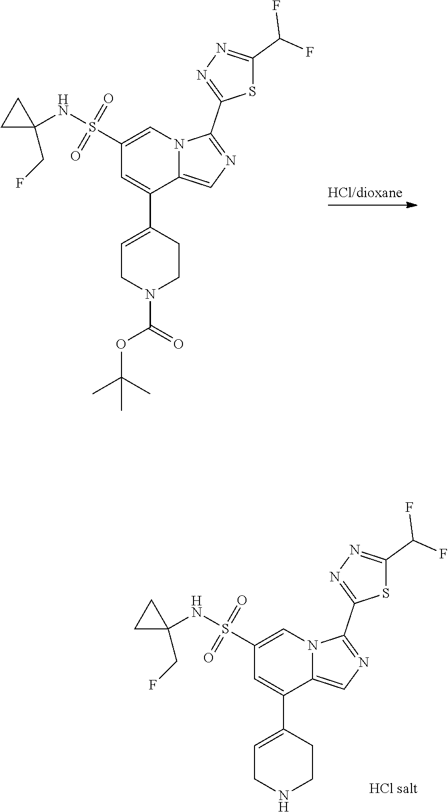 [SMILES: CC(C)(C)OC(=O)N1CC=C(c2cc(S(=O)(=O)NC3(CF)CC3)cn3c(-c4nnc(C(F)F)s4)ncc23)CC1 ; O=S(=O)(NC1(CF)CC1)c1cc(C2=CCNCC2)c2cnc(-c3nnc(C(F)F)s3)n2c1]

A solution of tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-3,6-dihydropyridine-1(2H)-carboxylate (20 mg, 34.21 μmol) in HCl/dioxane (4 M, 1 mL) was stirred at 25° C. for 1 h. The reaction mixture was concentrated under vacuum to give the crude product 3-(5-(difluoromethyl)-1,3,4-thiadiazol-2-yl)-N-(1-(fluoromethyl)cyclopropyl)-8-(1,2,3,6-tetrahydropyridin-4-yl)imidazo[1,5-a]pyridine-6-sulfonamide (25 mg, crude, HCl Salt) as a yellow solid. The crude product (5 mg) was purified by preparative HPLC (column: Phenomenex luna C18 150*25 mm*10 μm; mobile phase: A: 0.225% formic acid in water, B: MeCN; B %: 11%-41%, 10 min), then lyophilized to give the product with 85% purity. This material was further purified by preparative HPLC (column: Phenomenex luna C18 150*25 mm*10 μm; mobile phase: A: 0.1% trifluoroacetic acid in water, B: MeCN; B %: 11%-44%, 10 min) and lyophilized directly to give the product 3-(5-(difluoromethyl)-1,3,4-thiadiazol-2-yl)-N-(1-(fluoromethyl)cyclopropyl)-8-(1,2,3,6-tetrahydropyridin-4-yl)imidazo[1,5-a]pyridine-6-sulfonamide (3.06 mg, 4.86 μmol, 56.34% yield, TFA salt) as a yellow solid.

RT 0.343 min (method 3); m/z 485.2 (M+H)⁺ (ESI⁺), ¹H NMR (DMSO-d₆, 400 MHz): 9.86 (s, 1H), 8.93 (s, 1H), 8.92-8.90 (m, 1H), 8.11 (s, 1H), 7.69 (t, J=52.0 Hz, 1H), 7.32 (d, J=0.8 Hz, 1H), 6.49 (s, 1H), 4.24 (d, J=40.0 Hz, 2H), 3.89 (s, 2H), 3.43 (t, J=8.0 Hz, 2H), 2.80-2.72 (m, 2H), 0.90-0.85 (m, 2H), 0.84-0.74 (m, 2H).

### Preparation of Example 28

### 3-(5-(difluoromethyl)-1,3,4-thiadiazol-2-yl)-6-(N-(1-(fluoromethyl)cyclopropyl)sulfamoyl)imidazo [1,5-a]pyridin-8-yl)-N,N-dimethyl-3,6-dihydropyridine-1(2H)-carboxamide

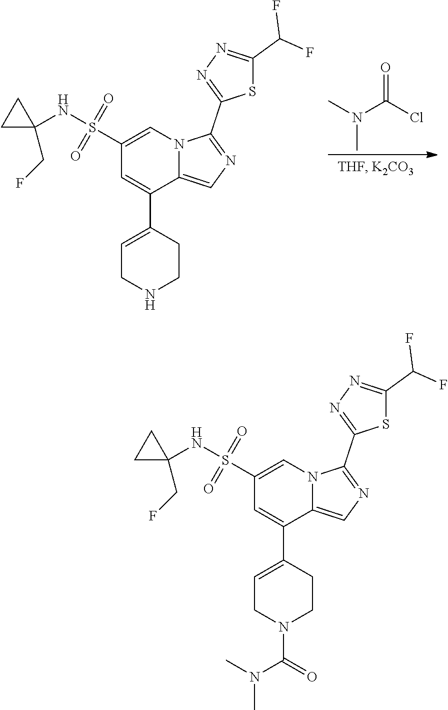

To a solution of 3-(5-(difluoromethyl)-1,3,4-thiadiazol-2-yl)-N-(1-(fluoromethyl)cyclopropyl)-8-(1,2,3,6-tetrahydropyridin-4-yl)imidazo[1,5-a]pyridine-6-sulfonamide (20 mg, 41.28 μmol, HCl salt) in THE (0.5 mL) and H₂O (0.25 mL) at 0° C. was added K₂CO₃ (17.11 mg, 123.83 μmol) and dimethylcarbamic chloride (6.66 mg, 61.92 μmol, 5.69 μL). The mixture was stirred at 25° C. for 1 h, then diluted with water (15 mL) and extracted with EtOAc (15 mL, 3×). The organic layer was washed with brine (20 mL), dried over Na₂SO₄, filtered and concentrated under vacuum to give 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethyl-3,6-dihydropyridine-1(2H)-carboxamide (23 mg, 39.30 μmol, 90.26% yield, 90% purity) as a yellow solid. 7 mg of the crude product were further purified by preparative HPLC (column: Phenomenex luna C18 150*25 mm*10 μm; mobile phase: A: 0.225% formic acid in water, B: MeCN; B %: 35%-65%, 10 min) and lyophilized directly to give 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethyl-3,6-dihydropyridine-1(2H)-carboxamide (3.81 mg, 6.51 μmol, 54.4% yield, 95% purity) as a yellow solid.

RT 0.458 min (method 3); m/z 556.3 (M+H)⁺ (ESI⁺), ¹H NMR (CDCl₃, 400 MHz): 10.14 (s, 1H), 7.88 (s, 1H), 7.22 (s, 1H), 7.09 (t, J=52.0 Hz, 1H), 6.38 (s, 1H), 5.54 (s, 1H), 4.29 (d, J=48.0 Hz, 1H), 4.08-4.06 (m, 2H), 3.55 (t, J=8.0 Hz, 2H), 2.92 (s, 6H), 2.70-2.68 (m, 2H), 1.17-1.13 (m, 2H), 0.90-0.87 (m, 2H).

### Preparation of Example 29

### 4-(1-chloro-3-(5-(difluoromethyl)-1,3,4-thiadiazol-2-yl)-6-(N-(1-(fluoromethyl)cyclopropyl)sulfamoyl) imidazo[1,5-a]pyridin-8-yl)-N,N-dimethyl-3,6-dihydropyridine-1(2H)-carboxamide

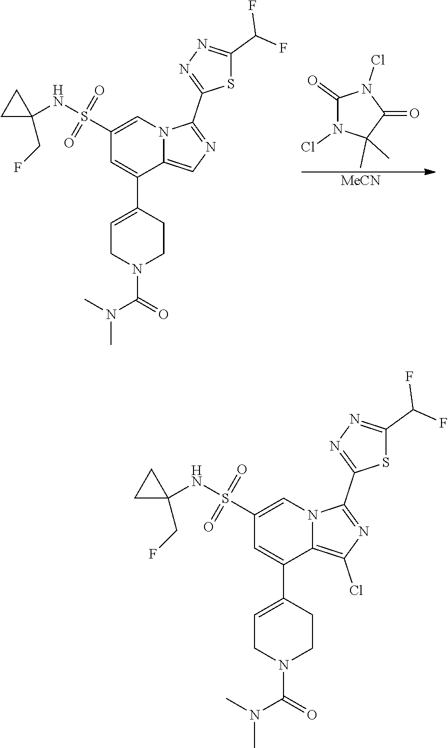 [SMILES: "MeCN" ; CN(C)C(=O)N1CC=C(c2cc(S(=O)(=O)NC3(CF)CC3)cn3c(-c4nnc(C(F)F)s4)ncc23)CC1 ; CC1(C)C(=O)N(Cl)C(=O)N1Cl ; CN(C)C(=O)N1CC=C(c2cc(S(=O)(=O)NC3(CF)CC3)cn3c(-c4nnc(C(F)F)s4)nc(Cl)c23)CC1]

To a mixture of 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethyl-3,6-dihydropyridine-1(2H)-carboxamide (16 mg, 28.80 μmol) in MeCN (0.5 mL) at 0° C. was added 1,3-dichloro-5,5-dimethylimidazolidine-2,4-dione (5.67 mg, 28.80 μmol). The mixture was stirred at 20° C. for 16 h and then concentrated under vacuum. The residue was purified by preparative HPLC (column: Phenomenex luna C18 150*25 mm*10 μm; mobile phase: A: 0.225% formic acid in water, B: MeCN; B %: 45%-75%, 10 min) and lyophilized directly to give the product 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethyl-3,6-dihydropyridine-1(2H)-carboxamide (2.77 mg, 4.51 μmol, 15.65% yield, 96% purity) as a yellow solid.

RT 0.479 min (method 3); m/z 590.3 (M+H)⁺ (ESI⁺), ¹H NMR (CDCl₃, 400 MHz): 10.13 (d, J=1.2 Hz, 1H), 7.12 (d, J=1.2 Hz, 1H), 7.09 (t, J=52.0 Hz, 1H), 5.89 (s, 1H), 5.54 (s, 1H), 4.29 (d, J=48.0 Hz, 2H), 4.02-3.99 (m, 2H), 3.56 (t, J=8.0 Hz, 2H), 2.91 (s, 6H), 2.62-2.53 (m, 2H), 1.17-1.13 (m, 2H), 0.90 (t, J=8.0 Hz, 2H).

### Preparation of Example 30

### tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)piperazine-1-carboxylate

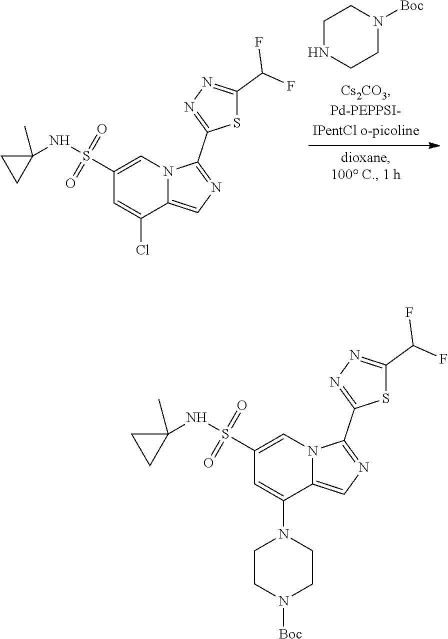 [SMILES: CC1(NS(=O)(=O)c2cc(Cl)c3cnc(-c4nnc(C(F)F)s4)n3c2)CC1 ; CC(C)(C)OC(=O)N1CCNCC1 ; CC1(NS(=O)(=O)c2cc(N3CCN(C(=O)OC(C)(C)C)CC3)c3cnc(-c4nnc(C(F)F)s4)n3c2)CC1]

To a mixture of 8-chloro-3-(5-(difluoromethyl)-1,3,4-thiadiazol-2-yl)-N-(1-methylcyclopropyl)imidazo[1,5-a]pyridine-6-sulfonamide (100 mg, 238.18 μmol) in dioxane (2 mL) was added tert-butyl piperazine-1-carboxylate (106.09 mg, 476.35 μmol), Cs₂CO₃ (155.21 mg, 476.35 μmol) and Pd-PEPPSI-IPentCl o-picoline (20.49 mg, 23.82 μmol) and the reaction mixture was degassed with N₂ (3×). The mixture was stirred at 100° C. for 1 h, then filtered and the filtrate was concentrated under vacuum. The resulting residue was purified by preparative TLC (Petroleum ether:EtOAc=0/1) to give the product tert-butyl 4-(3-(5-(difluoromethyl)-1,3,4-thiadiazol-2-yl)-6-(N-(1-methylcyclopropyl)sulfamoyl)imidazo[1,5-a]181yridine-8-yl)piperazine-1-carboxylate (130 mg, 182.57 μmol, 76.65% yield, 80% purity) as a yellow solid.

RT 0.653 min (method 3); m/z 514.0 (M+H)⁺ (ES⁺); ¹H NMR (CDCl₃, 400 MHz): 9.88 (s, 1H), 7.71 (s, 1H), 7.08 (t, J=53.6 Hz, 1H), 6.65 (s, 1H), 5.04 (s, 1H), 3.72-3.70 (m, 4H), 3.31-3.29 (m, 4H), 1.52 (s, 9H), 1.39 (s, 3H), 0.95-0.90 (m, 2H), 0.62-0.59 (m, 2H).

### Preparation of Example 31

### 3-(5-(difluoromethyl)-1,3,4-thiadiazol-2-yl)-N-(1-methylcyclopropyl)-8-(piperazin-1-yl)imidazo[1,5-a]pyridine-6-sulfonamide

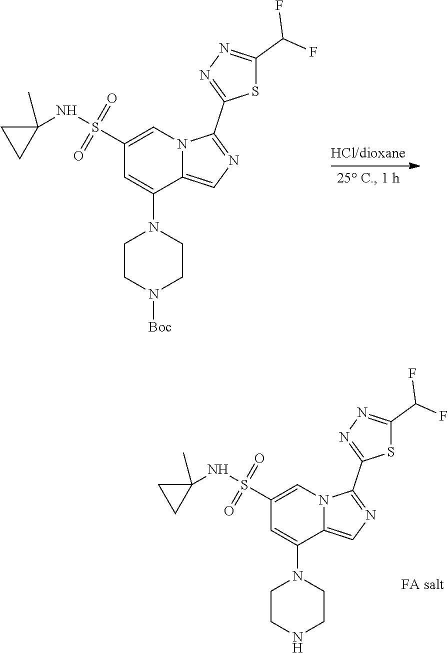 [SMILES: CC1(NS(=O)(=O)c2cc(N3CCN(C(=O)OC(C)(C)C)CC3)c3cnc(-c4nnc(C(F)F)s4)n3c2)CC1 ; CC1(NS(=O)(=O)c2cc(N3CCNCC3)c3cnc(-c4nnc(C(F)F)s4)n3c2)CC1]

A mixture of tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)piperazine-1-carboxylate (50 mg, 87.77 μmol) in HCl/dioxane (4 M, 2 mL) was stirred at 25° C. for 1 h. The reaction mixture was concentrated undervacuum to give a residue, which was purified by preparative HPLC (column: Phenomenex luna C18 150*25 mm*10 μm; mobile phase: A: 0.225% formic acid in water, B: MeCN; B %: 11%-41%, 10 min) and lyophilized directly to give the product 3-(5-(difluoromethyl)-1,3,4-thiadiazol-2-yl)-N-(1-methylcyclopropyl)-8-(piperazin-1-yl)imidazo[1,5-a]pyridine-6-sulfonamide (12.90 mg, 26.38 μmol, 30.05% yield, 96% purity, FA salt) as a yellow solid.

RT 0.360 min (method 3); m/z 470.1 (M+H)⁺ (ESI⁺), ¹H NMR (DMSO-d₆, 400 MHz): 9.58 (s, 1H), 8.43 (s, 1H), 8.25 (s, 1H), 7.99 (s, 1H), 7.67 (t, J=53.2 Hz, 1H), 6.67 (s, 1H), 3.26 (s, 4H), 3.01 (s, 4H), 1.15 (s, 3H), 0.73-0.71 (m, 2H), 0.50-0.38 (m, 2H).

### Preparation of Example 32

### 3-(5-(difluoromethyl)-1,3,4-thiadiazol-2-yl)-8-(4-isobutyrylpiperazin-1-yl)-N-(1-methylcyclopropyl)imidazo[1,5-a]pyridine-6-sulfonamide

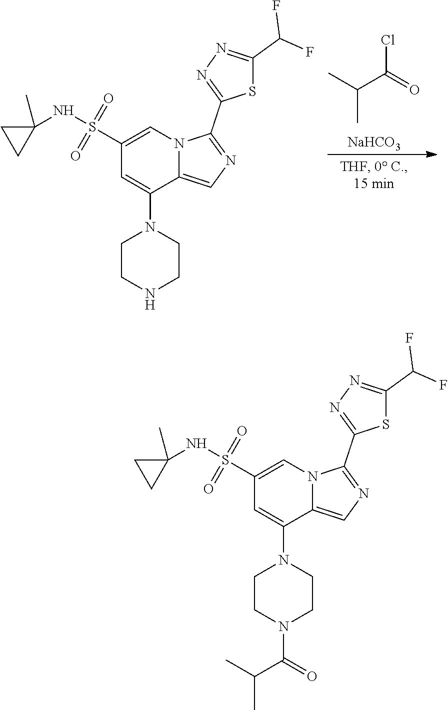

To a mixture of 3-(5-(difluoromethyl)-1,3,4-thiadiazol-2-yl)-N-(1-methylcyclopropyl)-8-(piperazin-1-yl)imidazo[1,5-a]pyridine-6-sulfonamide (30 mg, 63.89 μmol, FA salt) in THE (2 mL) and H₂O (1 mL) was added NaHCO₃ (aq., 53.67 mg, 638.94 μmol, 24.85 μL) and isobutyryl chloride (13.62 mg, 127.79 μmol, 13.35 μL) at 0° and the mixture was stirred at 0° C. for 15 min. The mixture was poured into water (7 mL) and extracted with EtOAc (12 mL; 2×). The combined organic layer was dried over Na₂SO₄, filtered and concentrated under vacuum to give a residue which was purified by preparative HPLC (column: Phenomenex luna C18 150*25 mm*10 μm; mobile phase: A: 0.225% formic acid in water, B: MeCN; B %: 41%-71%, 10 min)) and lyophilized directly to give the product 3-(5-(difluoromethyl)-1,3,4-thiadiazol-2-yl)-8-(4-isobutyrylpiperazin-1-yl)-N-(1-methylcyclopropyl)imidazo[1,5-a]pyridine-6-sulfonamide (5.98 mg, 10.97 μmol, 17.17% yield, 96.39% purity) as a yellow solid.

RT 0.498 min (method 3), m/z 540.2 (M+H)⁺ (ESI⁺), ¹H NMR (CDCl₃, 400 MHz) 9.90 (s, 1H), 7.73 (s, 1H), 7.08 (t, J=53.6 Hz, 1H), 6.66 (s, 1H), 5.08 (s, 1H), 3.91-3.81 (m, 4H), 4.00-3.65 (m, 2H), 3.65-3.32 (m, 2H), 2.88-2.85 (m, 1H), 1.40 (s, 3H), 1.20 (d, J=6.8 Hz, 6H), 0.95-0.92 (m, 2H), 0.65-0.55 (m, 2H).

### Preparation of Example 33

### tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-3,6-dihydropyridine-1(2H)-carboxylate

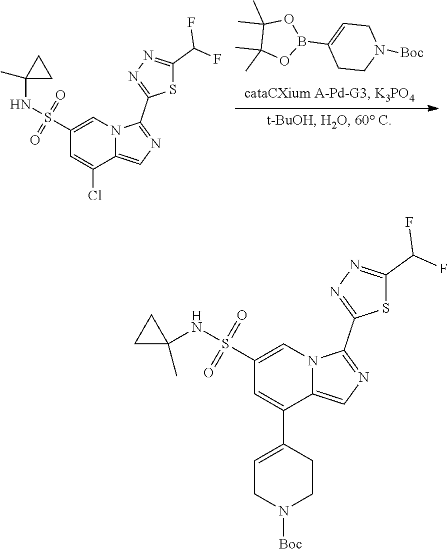

To a solution of 8-chloro-3-(5-(difluoromethyl)-1,3,4-thiadiazol-2-yl)-N-(1-methylcyclopropyl)imidazo[1,5-a]pyridine-6-sulfonamide (50 mg, 119.09 μmol) and tert-butyl 4-(4,4,5,5-tetramethyl-1,3,2-dioxaborolan-2-yl)-3,6-dihydropyridine-1(2H)-carboxylate (110.47 mg, 357.26 μmol) in t-BuOH (1 mL) and H₂O (0.1 mL) was added K₃PO₄ (30.33 mg, 142.91 μmol). The mixture was degassed with N₂ (3×). Then, cataCXium A-Pd-G3 (8.6 mg, 11.91 μmol) was added. The mixture was stirred at 60° C. for 16 h, cooled to room temperature and dissolved in MeOH (1 mL). The residue was purified by preparative HPLC (column: Phenomenex luna C18 150*25 mm*10 μm; mobile phase: A: 0.225% formic acid in water, B: MeCN; B %: 60%-90%, 10 min), then lyophilized to give the product tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-3,6-dihydropyridine-1(2H)-carboxylate (35.21 mg, 60.27 μmol, 50.61% yield, 97% purity) as a yellow solid.

RT 0.567 min (method 3); m/z 567.0 (M+H)⁺ (ESI⁺); ¹H NMR (CDCl₃, 400 MHz): 10.13 (s, 1H), 7.85 (s, 1H), 7.21 (s, 1H), 7.09 (t, J=53.6 Hz, 1H), 6.35 (s, 1H), 5.13 (s, 1H), 4.21-4.19 (m, 2H), 3.73 (t, J=5.2 Hz, 2H), 2.63-2.61 (m, 2H), 1.53 (s, 9H), 1.39 (s, 3H), 0.92 (t, J=6.0 Hz, 2H), 0.62 (t, J=6.4 Hz, 2H).

### Preparation of Example 34

### tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-3,6-dihydropyridine-1(2H)-carboxylate

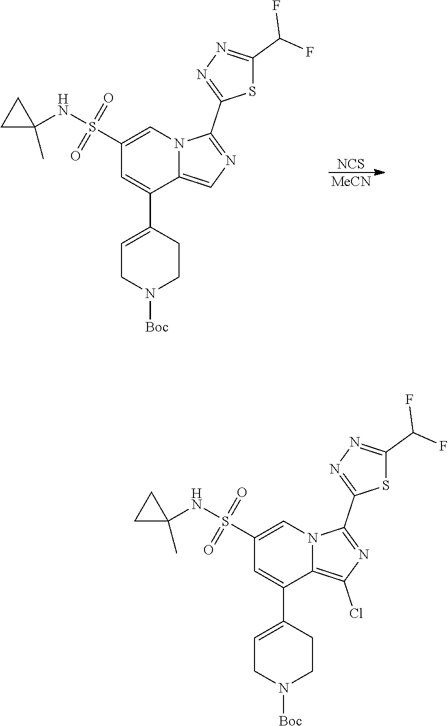 [SMILES: "MeCN" ; CC1(NS(=O)(=O)c2cc(C3=CCN(C(=O)OC(C)(C)C)CC3)c3cnc(-c4nnc(C(F)F)s4)n3c2)CC1 ; "NCS" ; CC1(NS(=O)(=O)c2cc(C3=CCN(C(=O)OC(C)(C)C)CC3)c3c(Cl)nc(-c4nnc(C(F)F)s4)n3c2)CC1]

To a solution of tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-3,6-dihydropyridine-1(2H)-carboxylate (140 mg, 247.06 μmol) in MeCN (1.5 mL) was added NCS (49.49 mg, 370.60 μmol) and the mixture was stirred at 20° C. for 16 h. The reaction was quenched with NaHCO₃ (10 mL). Then, the resulting mixture was extracted with EtOAc (30 mL; 3×). The combined organic layer was washed with brine (20 mL; 3×), dried over anhydrous Na₂SO₄, filtered and concentrated under reduced pressure to give 36 mg crude product. 15 mg of this crude product was purified by preparative HPLC (column: Phenomenex luna C18 150*25 mm*10 μm; mobile phase: A: 0.225% formic acid in water, B: MeCN; B %: 65%-95%, 10 min) and lyophilized to give the product tert-butyl-4-(1-chloro-3-(5-(difluoromethyl)-1,3,4-thiadiazol-2-yl)-6-(N-(1methylcyclopropyl)sulfamoyl)imidazo[1,5-a]pyridin-8-yl)-3,6-dihydropyridine-1(2H)-carboxylate (4.28 mg, 6.79 μmol, 6.6% yield, 95.3% purity) as a yellow solid.

RT 0.598 min (method 3); m/z 601.0 (M+H)⁺ (ESI⁺); ¹H NMR (CDCl₃, 400 MHz): 10.12 (d, J=1.4 Hz, 1H), 7.11 (d, J=1.2 Hz, 1H), 7.09 (t, J=53.6 Hz, 1H), 5.88 (s, 1H), 5.15 (s, 1H), 4.16-4.14 (m, 2H), 3.75 (t, J=5.2 Hz, 2H), 2.58-2.39 (m, 2H), 1.53 (s, 9H), 1.39 (s, 3H), 0.92 (t, J=6.4 Hz, 2H), 0.62 (t, J=6.8 Hz, 2H).

### Preparation of Example 35

### 1-chloro-3-(5-(difluoromethyl)-1,3,4-thiadiazol-2-yl)-N-(1-methylcyclopropyl)-8-(1,2,3,6-tetrahydropyridin-4-yl)imidazo[1,5-a]pyridine-6-sulfonamide formate

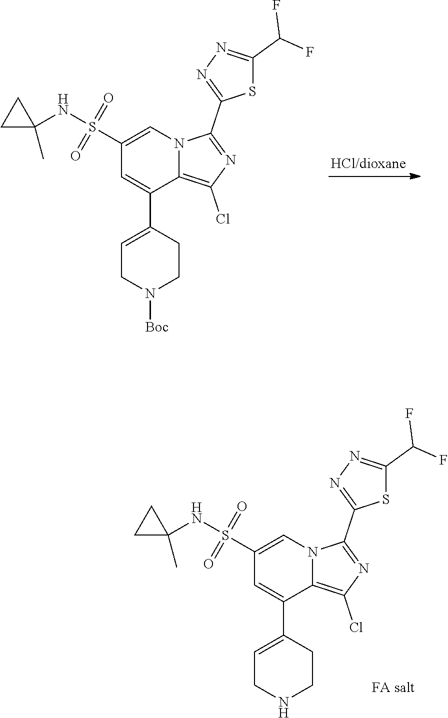 [SMILES: CC1(NS(=O)(=O)c2cc(C3=CCN(C(=O)OC(C)(C)C)CC3)c3c(Cl)nc(-c4nnc(C(F)F)s4)n3c2)CC1 ; CC1(NS(=O)(=O)c2cc(C3=CCNCC3)c3c(Cl)nc(-c4nnc(C(F)F)s4)n3c2)CC1]

A solution of tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-3,6-dihydropyridine-1(2H)-carboxylate (35 mg, 58.23 μmol) in HCl/dioxane (4 M, 0.2 mL) was stirred at 20° C. for 1 h. The mixture was concentrated to give 35 mg crude product. 10 mg of this crude product was purified by preparative HPLC (column: Phenomenex luna C18 150*25 mm*10 μm; mobile phase: A: 0.225% formic acid in water, B: MeCN; B %: 15%-45%, 10 min) and lyophilized to give the product 1-chloro-3-(5-(difluoromethyl)-1,3,4-thiadiazol-2-yl)-N-(1-methylcyclopropyl)-8-(1,2,3,6-tetrahydropyridin-4-yl)imidazo[1,5-a]pyridine-6-sulfonamide formate (3.1 mg, 5.44 μmol, 32.7% yield, 96% purity, FA salt) as a yellow solid.

RT 0.376 min (method 3); m/z 501.3 (M+H)⁺ (ESI⁺); ¹H NMR (DMSO-d₆, 400 MHz): 9.83 (d, J=1.6 Hz, 1H), 8.73-8.42 (m, 1H), 8.25 (s, 1H), 7.69 (t, J=53.6 Hz, 1H), 7.12 (d, J=1.2 Hz, 1H), 5.92 (s, 1H), 3.52-3.50 (m, 2H), 3.11 (t, J=5.6 Hz, 2H), 2.34 (s, 2H), 1.19 (s, 3H), 0.73 (t, J=6.4 Hz, 2H), 0.49-0.46 (m, J, 2H).

### Preparation of Example 36

### 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-N,N-dimethyl-3,6-dihydropyridine-1(2H)-carboxamide

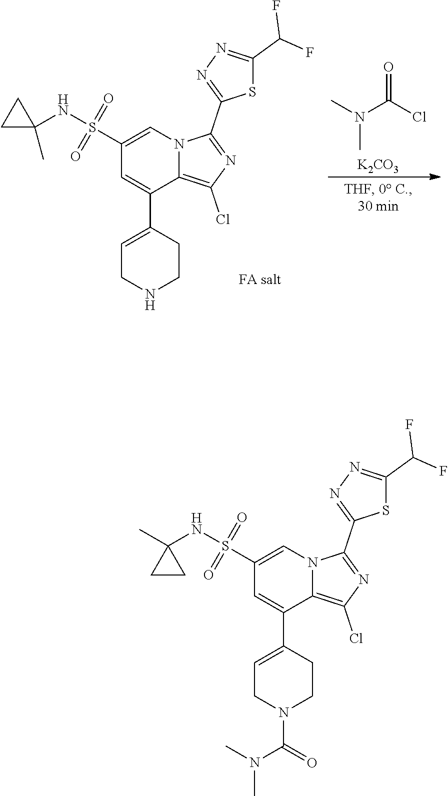

To a solution of 1-chloro-3-(5-(difluoromethyl)-1,3,4-thiadiazol-2-yl)-N-(1-methylcyclopropyl)-8-(1,2,3,6-tetrahydropyridin-4-yl)imidazo[1,5-a]pyridine-6-sulfonamide (20 mg, 36.56 μmol, FA salt) in THF (0.6 mL) and H₂O (0.3 mL) was added K₂CO₃ (16.55 mg, 119.77 μmol) and dimethylcarbamic chloride (6.44 mg, 59.88 μmol, 5.50 μL). The mixture was stirred at 0° C. for 1 h, diluted with H₂O (5 mL) and extracted with EtOAc (5 mL, 3×). The organic layer was washed with brine (5 mL, 2×), dried over anhydrous Na₂SO₄, filtered and concentrated under reduced pressure. The crude product was purified by preparative HPLC (column: Phenomenex luna C18 150*25 mm*10 μm; mobile phase: A: 0.225% formic acid in water, B: MeCN; B %: 48%-78%, 10 min) and lyophilized to give the product 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-N,N-dimethyl-3,6-dihydropyridine-1(2H)-carboxamide (5.38 mg, 9.31 μmol, 25.46% yield, 99% purity) as a yellow solid.

RT 0.498 min (method 3); m/z 572.4 (M+H)⁺ (ESI⁺); ¹H NMR (DMSO-d₆, 400 MHz): 10.12 (d, J=0.8 Hz, 1H), 7.12 (d, J=0.8 Hz, 1H), 7.09 (t, J=53.6 Hz, 1H), 5.88 (s, 1H), 5.19 (s, 1H), 4.01-3.99 (m, 2H), 3.57 (t, J=5.2 Hz, 2H), 2.91 (s, 6H), 2.57-2.54 (m, 2H), 1.39 (s, 3H), 0.92 (t, J=6.0 Hz, 2H), 0.62 (t, J=5.6 Hz, 2H).

### Preparation of Example 37

### 1-chloro-3-(5-(difluoromethyl)-1,3,4-thiadiazol-2-yl)-N-(1-(fluoromethyl)cyclopropyl)-8-(2-oxa-7-azaspiro[3.5]nonan-7-yl)imidazo[1,5-a]pyridine-6-sulfonamide

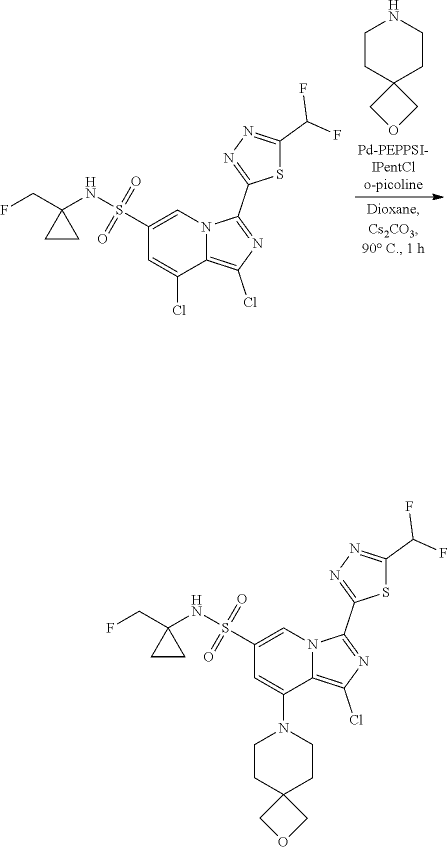

To a mixture of 1,8-dichloro-3-(5-(difluoromethyl)-1,3,4-thiadiazol-2-yl)-N-(1-(fluoromethyl)cyclopropyl)imidazo[1,5-a]pyridine-6-sulfonamide (200 mg, 0.423 mmol) in 1,4-dioxane (4 mL) was added 2-oxa-7-azaspiro[3.5]nonane (162 mg, 1.27 mmol), Cs₂CO₃ (414 mg, 1.27 mmol) and Pd-PEPPSI-IPent Cl o-picoline (36 mg, 0.0423 mmol). The reaction mixture was degassed with N₂ (3×) and stirred at 90° C. for 1 h. Then, it was filtered and the filtrate was concentrated under vacuum. The resulting residue was purified by preparative TLC (Petroleum:EtOAc=3/1) to give a crude product (32 mg) which was further triturated with MeOH (2 mL) at 20° C. for 10 min to give the product 1-chloro-3-(5-(difluoromethyl)-1,3,4-thiadiazol-2-yl)-N-(1-(fluoromethyl)cyclopropyl)-8-(2-oxa-7-azaspiro[3.5]nonan-7-yl)imidazo[1,5-a]pyridine-6-sulfonamide (25.55 mg, 44.9 μmol, 10.61% yield, 100% purity) as a yellow solid.

RT 0.515 min (method 3); m/z 563.0 (M+H)⁺ (ESI⁺); ¹H NMR (CDCl₃, 400 MHz) 9.87 (s, 1H), 7.07 (t, J=53.6 Hz, 1H), 6.69 (s, 1H), 5.58 (s, 1H), 4.53 (s, 4H), 4.26 (d, J=48.4 Hz, 2H), 3.29-2.94 (m, 4H), 2.17 (m, 4H), 1.17-1.13 (m, 2H), 0.89-0.86 (m, 2H).

### Preparation of Example 38

### 1-chloro-3-(5-(difluoromethyl)-1,3,4-thiadiazol-2-yl)-N-(1-methylcyclopropyl)-8-(2-oxa-7-azaspiro[3.5]nonan-7-yl)imidazo[1,5-a]pyridine-6-sulfonamide

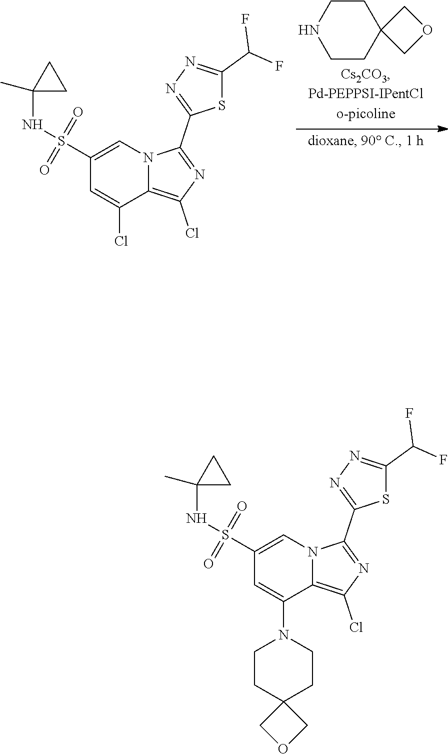 [SMILES: CC1(NS(=O)(=O)c2cc(Cl)c3c(Cl)nc(-c4nnc(C(F)F)s4)n3c2)CC1 ; C1CC2(CCN1)COC2 ; CC1(NS(=O)(=O)c2cc(N3CCC4(CC3)COC4)c3c(Cl)nc(-c4nnc(C(F)F)s4)n3c2)CC1]

To a mixture of 1,8-dichloro-3-(5-(difluoromethyl)-1,3,4-thiadiazol-2-yl)-N-(1-methylcyclopropyl)imidazo[1,5-a]pyridine-6-sulfonamide (40 mg, 88.05 μmol) in dioxane (1 mL) was added 2-oxa-7-azaspiro[3.5]nonane (22.40 mg, 176.09 μmol), Cs₂CO₃ (86.06 mg, 264.14 μmol) and Pd-PEPPSI-IPentCl o-picoline (7.58 mg, 8.80 μmol). The reaction mixture was degassed with N₂ (3×) and the mixture was stirred at 90° C. for 1 h. Then, it was filtered and filtrate was concentrated under vacuum. The residue was purified by preparative TLC (petroleum ether:EtOAc=0:1) and lyophilized to give the product 1-chloro-3-(5-(difluoromethyl)-1,3,4-thiadiazol-2-yl)-N-(1-methylcyclopropyl)-8-(2-oxa-7-azaspiro[3.5]nonan-7-yl)imidazo[1,5-a]pyridine-6-sulfonamide (15.58 mg, 27.55 μmol, 31.29% yield, 96.39% purity) as a yellow solid.

RT 0.509 min (method 3); m/z 545.2 (M+H)⁺ (ESI⁺); ¹H NMR (CDCl₃, 400 MHz): 9.87 (s, 1H), 7.68 (t, J=53.2 Hz, 1H), 6.70 (s, 1H), 5.09 (s, 1H), 4.54 (s, 4H), 3.10-3.05 (m, 4H), 2.25-2.10 (m, 4H), 1.39 (s, 3H), 0.94-0.91 (m, 2H), 0.66-0.59 (m, 2H).

### Preparation of Example 39

### 3-(5-(difluoromethyl)-1,3,4-thiadiazol-2-yl)-N-(1-methylcyclopropyl)-8-(1,2,3,6-tetrahydropyridin-4-yl)imidazo[1,5-a]pyridine-6-sulfonamide

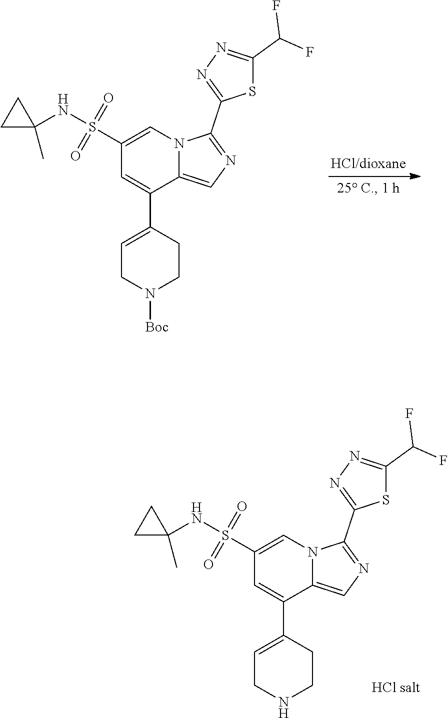 [SMILES: CC1(NS(=O)(=O)c2cc(C3=CCN(C(=O)OC(C)(C)C)CC3)c3cnc(-c4nnc(C(F)F)s4)n3c2)CC1 ; CC1(NS(=O)(=O)c2cc(C3=CCNCC3)c3cnc(-c4nnc(C(F)F)s4)n3c2)CC1]

A mixture of tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-5,6-dihydropyridine-1(2H)-carboxylate (40 mg, 70.59 μmol) in HCl/dioxane (4 M, 1.5 mL) was stirred at 25° C. for 1 h. The reaction mixture was concentrated under vacuum to give the product 3-(5-(difluoromethyl)-1,3,4-thiadiazol-2-yl)-N-(1-methylcyclopropyl)-8-(1,2,3,6-tetrahydropyridin-4-yl)imidazo[1,5-a]pyridine-6-sulfonamide (45 mg, crude, HCl salt) as a yellow solid.

RT 0.351 min (method 3); m/z 467.1 (M+H)⁺ (ESI⁺), ¹H NMR (DMSO-d₆, 400 MHz): 9.88 (s, 1H), 9.27 (d, J=0.8 Hz, 1H), 8.58 (s, 1H), 8.13 (s, 1H), 7.69 (t, J=53.2 Hz, 1H), 7.38 (d, J=0.8 Hz, 1H), 6.49 (s, 1H), 3.92-3.87 (m, 2H), 3.45-3.35 (m, 2H), 2.85-2.72 (m, 2H), 1.18 (s, 3H), 0.74-0.72 (m, 2H), 0.49-0.45 (m, 2H).

### Preparation of Example 40

### 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethyl-5,6-dihydropyridine-1(2H)-carboxamide

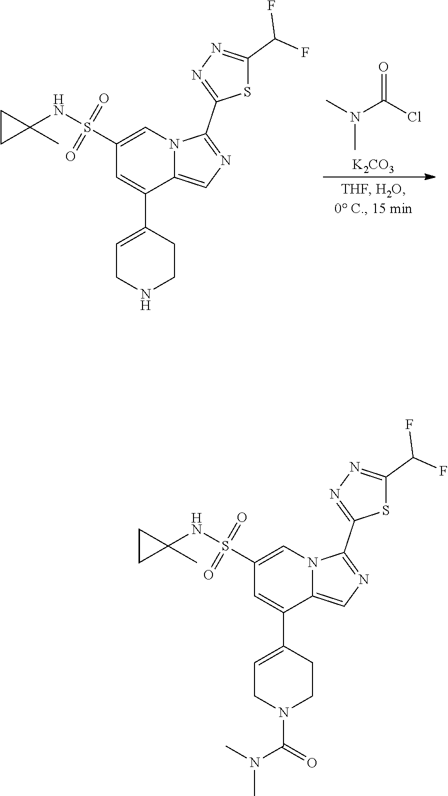

To a mixture of 3-(5-(difluoromethyl)-1,3,4-thiadiazol-2-yl)-N-(1-methylcyclopropyl)-8-(1,2,3,6-tetrahydropyridin-4-yl)imidazo[1,5-a]pyridine-6-sulfonamide (45 mg, 96.46 μmol, HCl salt) in THE (2 mL) and H₂O (1 mL) was added K₂CO₃ (39.99 mg, 289.37 μmol) and dimethylcarbamic chloride (15.56 mg, 144.69 μmol, 13.30 μL) at 0° C. The mixture was stirred at 0° C. for 15 min, poured into water (10 mL) and extracted with EtOAc (15 mL; 2×). Then, the combined organic layer was dried over Na₂SO₄, filtered and concentrated under vacuum. The resulting residue was purified by preparative HPLC (column: Phenomenex luna C18 150*25 mm*10 μm; mobile phase: A: 0.225% formic acid in water, B: MeCN; B %: 40%-70%, 10 min) and lyophilized directly to give the product 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethyl-5,6-dihydropyridine-1(2H)-carboxamide (12.1 mg, 22.06 μmol, 22.87% yield, 98% purity) as a yellow solid.

RT 0.494 min (method 3); m/z 538.1 (M+H)⁺ (ESI⁺), ¹H NMR (CDCl₃, 400 MHz): 10.13 (s, 1H), 7.87 (s, 1H), 7.22 (d, J=1.2 Hz, 1H), 7.09 (t, J=53.2 Hz, 1H), 6.38 (s, 1H), 5.09 (s, 1H), 4.10-3.95 (m, 2H), 3.65-3.45 (m, 2H), 2.92 (s, 6H), 2.70-2.58 (m, 2H), 1.39 (s, 3H), 0.93-0.91 (m, 2H), 0.68-0.58 (m, 2H).

The following general procedures apply to the synthesis of the compounds described in the following.
General procedure 1 (Buchwald coupling): To a solution of the chloro compound (1.00 eq) in 1,4-dioxane (500 mg/mL) was added the amine substrate (1 to 3 eq, free base or salt) and Cs₂CO₃ (2 to 4 eq). The mixture was degassed with N₂ (3×) or placed in a glove box. Then, Pd-PEPPSI-IPentCl o-picoline (0.05 to 0.1 eq) was added. The mixture was stirred at 80 to 100° C. for 1 to 16 h under N₂ outside of the glove box. Then, the reaction mixture was cooled to room temperature, diluted with H₂O and extracted with EtOAc (3×). The combined organic layers were washed with brine, dried over anhydrous Na₂SO₄, filtered and concentrated under reduced pressure. The resulting residue was purified by reverse preparative HPLC (reverse phase) or preparative TLC or SiO₂ column chromatography (normal phase) to give the corresponding product.
General procedure 2 (Boc cleavage): To a solution of the Boc-protected compound in DCM (0.1 g/mL) was added TFA (1/5 to 1/3 of the DCM volume) or HCl/Dioxane (4N, 1/5 to 1/3 of the DCM volume) at 0° C. and the mixture was stirred at 0° C. for 2 to 16 h. Then, the mixture was concentrated under reduce pressure. The resulting residue was purified by reverse preparative HPLC (reverse phase) or preparative TLC or SiO₂ column chromatography (normal phase) to give the corresponding product.
General procedure 3 (Bromination and iodination): To a mixture of the substrate (1 eq) in CHCl₃ (0.01 to 0.1 g/mL) or MeCN (0.01 to 0.1 g/mL) was added NBS (0.8 to 1.1 eq) or NIS (0.8 to 1.1 eq) in CHCl₃ (0.01 to 0.1 g/mL) or MeCN (0.01 to 0.1 g/mL) dropwise at 0° C. The mixture was stirred at room temperature for 0.5 h and concentrated under reduced pressure. The resulting residue was purified by reverse preparative HPLC (reverse phase) or preparative TLC or SiO₂ column chromatography (normal phase) to give the corresponding product.
General procedure 4 (Amine ester exchange): To a mixture of the ester substrate (1 eq) in EtOH (10 to 100 mg/mL) and H₂O (1/10 to 1/3 of the EtOH volume) was added the amine substrate (1 to 10 eq, free base or HCl salt). The mixture was stirred at 20 to 100° C. for 2 to 20 h. The reaction mixture was concentrated under reduced pressure. The resulting residue was purified by reverse preparative HPLC (reverse phase) or preparative TLC or SiO₂ column chromatography (normal phase) to give the corresponding product.
General procedure 5 (Amide formation using a peptide coupling reagent): To a solution of the acid substrate (1 eq) in DMF (20 to 100 mg/mL) was added HATU (1 to 2 eq) and DIEA (1.5 to 2 eq). The mixture was stirred at 20° C. for 30 min and the amine substrate (1.2 to 1.5 eq, free base or HCl salt) was added. The reaction was stirred at 20° C. for 1 to 16 h, then diluted with ice/H₂O and extracted with EtOAc (3×). The combined organic layers were washed with brine, dried over anhydrous Na₂SO₄, filtered and the filtrate was concentrated under reduced pressure. The resulting residue was purified by reverse preparative HPLC (reverse phase) or preparative TLC or SiO₂ column chromatography (normal phase) to give the corresponding product.
General procedure 6 (Suzuki coupling with Xantphos Pd G4): To a solution of the bromide or iodide substrate (1 eq), the borate ester or borate acid substrate (1 to 4 eq) and Cs₂CO₃ (2 to 4 eq) in dioxane (10 to 100 mg/mL) and H₂O (1/10 to 1/5 of the dioxane volume) was added Xantphos Pd G4 (0.1 to 0.2 eq). The reaction mixture was degassed and purged with N₂ (3×), stirred at 80 to 100° C. for 2 to 16 h and concentrated under reduced pressure. The resulting residue was purified by reverse preparative HPLC (reverse phase) or preparative TLC or SiO₂ column chromatography (normal phase) to give the corresponding product.

Alternatively, after stirring at 80 to 100° C. for 2 to 16 h, the reaction mixture was cooled to room temperature, diluted with H₂O and extracted with EtOAc (3×). The combined organic layers were washed with brine, dried over anhydrous Na₂SO₄, filtered and concentrated under reduced pressure. The resulting residue was purified by reverse preparative HPLC (reverse phase) or preparative TLC or SiO₂ column chromatography (normal phase) to give the corresponding product.
General procedure 7 (Hydrogenation): To a solution of the olefin substrate (1 eq) in THE (2.5 to 50 g/mL) was added Pd/C (m=1/10 to 1/5 mg of substrate mass, 10% purity) under N₂. The suspension was degassed under vacuum and purged with H₂ (3×). The mixture was stirred under a H₂ atmosphere (15 psi) at 20 to 50° C. for 2 to 16 h before it was filtered and the filtrate was concentrated under reduced pressure. The resulting residue was purified by reverse preparative HPLC (reverse phase) or preparative TLC or SiO₂ column chromatography (normal phase) to give the corresponding product.
General procedure 8 (Sonogashira coupling 1): To a solution of the bromide or iodide substrate (1 eq) in DMF or dioxane or DMF/MeOH (1 to 100 mg/mL) was added CuI (0.05 to 0.3 eq), K₂CO₃ (2 to 4 eq) or TEA (1/2 to 1 V of solvent) and Pd(PPh₃)₂Cl₂ or Pd(dppf)Cl₂ (0.05 to 0.3 eq). The reaction mixture was degassed and purged with N₂ (3×). Then, the alkyne substrate (1 to 3 eq) was added to the mixture. The reaction mixture was stirred at 80 to 150° C. for 0.5 to 16 h under N₂ atmosphere before it was poured into water and extracted with EtOAc (3×). The combined organic layers were dried over Na₂SO₄, filtered and concentrated under reduced pressure. The resulting residue was purified by reverse preparative HPLC (reverse phase) or preparative TLC or SiO₂ column chromatography (normal phase) to give the corresponding product.
General procedure 9 (Saponification): To the mixture of the ester substrate (1 eq) in MeOH (10 to 200 mg/mL) and H₂O (1/3 to 1/1 of the MeOH volume) was added LiOH or LiOH—H₂O (2 to 10 eq). The mixture was stirred at 20 to 80° C. for 1 to 16 h. Work up procedure 1: The pH of the reaction mixture was adjusted to 1 to 6 by addition of an aqueous solution of hydrochloric acid (1N) followed by extraction with EtOAc (3×). The combined organic layers were washed with brine, dried over Na₂SO₄, filtered and concentrated under reduced pressure to give the crude product which was used in the next step without further purification. Work up 2: The pH of the reaction mixture was adjusted to 1 to 6 by addition of an aqueous solution of hydrochloric acid (1N). The resulting residue was purified by reverse preparative HPLC (reverse phase) or preparative TLC or SiO₂ column chromatography (normal phase) to give the corresponding product.
General procedure 10 (Sonogashira coupling 2): To a solution of the bromide or iodide substrate (1 eq) in MeCN (50 mg/mL) was added P(t-Bu)₃ (0.2 eq, 10% w/w in hexane), TEA or DABCO (2 eq) and allyl(chloro)palladium (0.1 eq). The reaction mixture was degassed and purged with N₂ (3×) before the alkyne substrate (2 eq) was added to the mixture. The reaction mixture was stirred at room temperature for 1 to 16 h under a N₂ atmosphere and concentrated under reduced pressure. The resulting residue was purified by reverse preparative HPLC (reverse phase) or preparative TLC or SiO₂ column chromatography (normal phase) to give the corresponding product.

Alternatively, after stirring, the reaction mixture was diluted with H₂O and extracted with EtOAc (3×). The combined organic layers were washed with brine, dried over anhydrous Na₂SO₄, filtered and concentrated under reduced pressure. The resulting residue was purified by reverse preparative HPLC (reverse phase) or preparative TLC or SiO₂ column chromatography (normal phase) to give the corresponding product.
General procedure 11 (TBS cleavage): To a solution of the TBS protected compound in MeOH (0.5 g/mL) was added KF (2 eq). The mixture was stirred at room temperature for 1 to 16 h, then concentrated under reduced pressure. The resulting residue was purified by reverse preparative HPLC (reverse phase) or preparative TLC or SiO₂ column chromatography (normal phase) to give the corresponding product.
General procedure 12 (Amide formation using an acyl chloride): To a solution of the amine substrate (1 eq) in THE (20 to 100 mg/mL) was added NaHCO₃ (saturated solution, 20 to 100 mg/mL) or TEA (1.5 to 2 eq). The mixture was stirred at 0° C. for 2 min and the acyl chloride (1.2 to 1.5 eq) was added to the mixture. The reaction was stirred at 20° C. for 0.5 to 1 h, then diluted with ice-H₂O and extracted with EtOAc. The combined organic layer was washed with brine, dried over anhydrous Na₂SO₄, filtered and the filtrate was concentrated under reduced pressure. The resulting residue was purified by reverse preparative HPLC (reverse phase) or preparative TLC or SiO₂ column chromatography (normal phase) to give the corresponding product.
General procedure 13 (Stille coupling): To a solution of the tin substrate (1 eq) in dioxane (V=15 mg/mL) was added the halogenated substrate (1.5 eq), K₂CO₃ (2 eq) and Pd(dppf)Cl₂ (0.1 eq). The reaction mixture was degassed and purged with N₂ (3×). The reaction mixture was stirred at 80° C. for 16 h under N₂ atmosphere then, poured in water and extracted with EtOAc. The combined organic layer was dried over Na₂SO₄, filtered and concentrated under reduced pressure. The resulting residue was purified by reverse preparative HPLC (reverse phase)
General procedure 14 (Ester formation): A solution of the acid substrate (1 eq), DMAP (0.5 eq) and DCC (2 eq) in DCM (20 to 100 mg/mL) was stirred room temperature for 1 to 3 h and the alcohol substrate (1 to 2 eq) was added. The mixture was stirred at room temperature for 1 to 16 h. The resulting mixture was diluted with ice/H₂O and extracted with EtOAc (3×). The combined organic layers were washed with brine, dried over anhydrous Na₂SO₄, filtered and the filtrate was concentrated under reduced pressure. The resulting residue was purified by reverse preparative HPLC (reverse phase) or preparative TLC or SiO₂ column chromatography (normal phase) to give the corresponding product.

### Preparation of Example 41.a

### 8-chloro-3-(5-(difluoromethyl)-1,3,4-thiadiazol-2-yl)-N-(1-methylcyclopropyl)imidazo[1,2-a]pyridine-6-sulfonamide

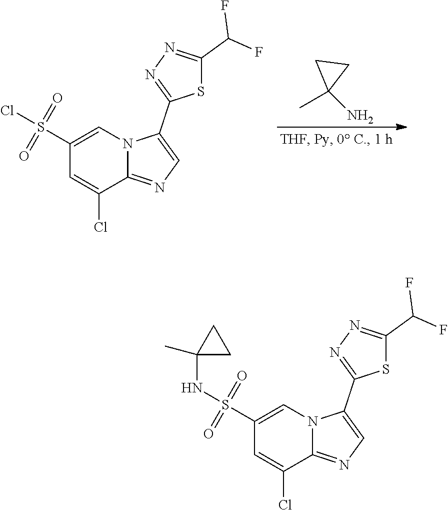

To the mixture of 1-methylcyclopropanamine (78.20 mg, 726.90 μmol, HCl salt) in NaHCO₃ (aq., sat., 3 mL) was added drop-wise 8-chloro-3-(5-(difluoromethyl)-1,3,4-thiadiazol-2-yl)imidazo[1,2-a]pyridine-6-sulfonyl chloride (140 mg, 363.45 μmol) in THE (1.5 mL) at 0° C. The mixture was stirred at 15° C. for 2 h. The reaction mixture was quenched by H₂O (30 mL) and extracted with EtOAc (30 mL, 3×). The combined organic layer was washed with brine (30 mL), dried over Na₂SO₄, filtered and concentrated under reduced pressure. The resulting residue was purified by flash silica gel chromatography (ISCO®; 4 g SepaFlash® Silica Flash Column, Eluent 20-40% Ethyl acetate/Petroleum@75 mL/min) to give the product 8-chloro-3-(5-(difluoromethyl)-1,3,4-thiadiazol-2-yl)-N-(1-methylcyclopropyl)imidazo[1,2-a]pyridine-6-sulfonamide (50 mg, 119.09 μmol, 32.77% yield) as a white solid.

RT 0.809 min (Method 1); m/z 420.1 (M+H)⁺ (ESI⁺); ¹H NMR (DMSO-d₆, 400 MHz) 9.99 (d, J=1.2 Hz, 1H), 8.81 (s, 1H), 8.58 (s, 1H), 7.94 (d, J=1.2 Hz, 1H), 7.73 (t, J=53.2 Hz, 1H), 1.17-1.20 (m, 3H), 0.67-0.76 (m, 2H), 0.45-0.53 (m, 2H).

### Preparation of Example 41

### 4-(3-(5-(difluoromethyl)-1,3,4-thiadiazol-2-yl)-6-(N-(1-methylcyclopropyl)sulfamoyl)imidazo[1,2-a]pyridin-8-yl)-N,N-dimethylpiperazine-1-carboxamide

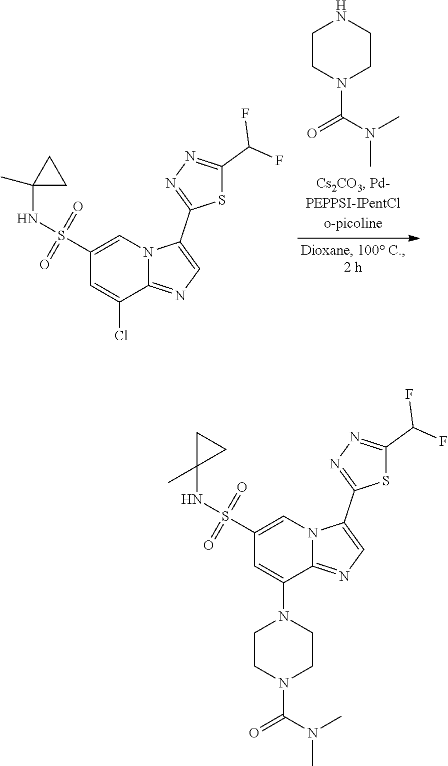

To a solution of N,N-dimethylpiperazine-1-carboxamide (14.98 mg, 95.27 μmol) in dioxane (0.5 mL) was added 8-chloro-3-(5-(difluoromethyl)-1,3,4-thiadiazol-2-yl)-N-(1-methylcyclopropyl)imidazo[1,2-a]pyridine-6-sulfonamide (20 mg, 47.64 μmol), Cs₂CO₃ (31.04 mg, 95.27 μmol) and Pd-PEPPSI-IPentCl o-picoline (2.05 mg, 2.38 μmol) in a glove box. The mixture was stirred outside of the glove box at 100° C. for 2 h under Ar., cooled down to room temperature and concentrated under reduced pressure. The resulting residue was diluted with MeOH (3 mL), filtered and the filtrate was purified by preparative HPLC (column: Unisil 3-100 C18 Ultra 150*50 mm*3 μm; mobile phase: A: 0.225% formic acid in water, B: MeCN; B %: 32%-62%, 7 min) and lyophilized directly to afford the product 4-(3-(5-(difluoromethyl)-1,3,4-thiadiazol-2-yl)-6-(N-(1-methylcyclopropyl)sulfamoyl)imidazo[1,2-a]pyridin-8-yl)-N,N-dimethylpiperazine-1-carboxamide (3.05 mg, 5.42 μmol, 11.37% yield, 96% purity) as a light yellow solid.

RT 0.896 min (Method 1); m/z 541.1 (M+H)⁺ (ESI⁺); ¹H NMR (DMSO-d₆, 400 MHz): 9.67 (s, 1H), 8.63 (s, 1H), 8.32-8.50 (m, 1H), 7.71 (t, J=53.2 Hz, 1H), 7.02 (s, 1H), 3.64 (br, 4H), 3.35-3.37 (m, 4H), 2.81 (s, 6H), 1.14 (s, 3H), 0.66-0.77 (m, 2H), 0.39-0.52 (m, 2H).

### Preparation of Example 42

### 3-(5-(difluoromethyl)-1,3,4-thiadiazol-2-yl)-N-(1-methylcyclopropyl)-8-(2-oxa-7-azaspiro[3.5]nonan-7-yl)imidazo[1,2-a]pyridine-6-sulfonamide

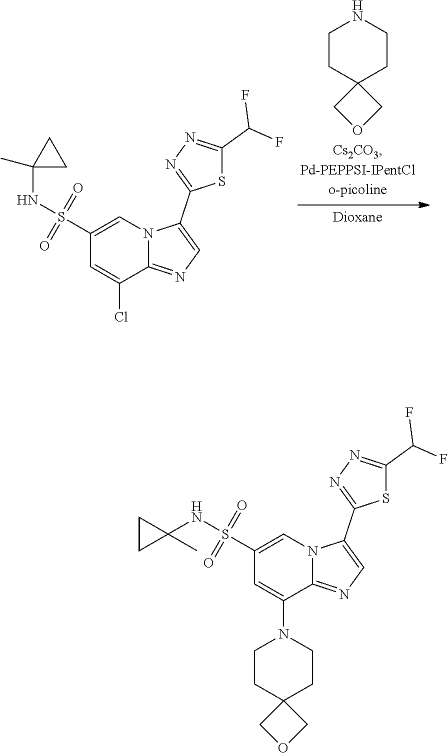 [SMILES: CC1(NS(=O)(=O)c2cc(Cl)c3ncc(-c4nnc(C(F)F)s4)n3c2)CC1 ; C1CC2(CCN1)COC2 ; CC1(NS(=O)(=O)c2cc(N3CCC4(CC3)COC4)c3ncc(-c4nnc(C(F)F)s4)n3c2)CC1]

To a solution of 8-chloro-3-(5-(difluoromethyl)-1,3,4-thiadiazol-2-yl)-N-(1-methylcyclopropyl)imidazo[1,2-a]pyridine-6-sulfonamide (30 mg, 69.63 μmol) in dioxane (0.5 mL) was added 2-oxa-7-azaspiro[3.5]nonane (25.19 mg, 104.45 μmol, TFA salt), Cs₂CO₃ (68.06 mg, 208.89 μmol). In glove box, Pd-PEPPSI-IPentCl o-picoline (3.00 mg, 3.48 μmol) was added. The mixture was then stirred at 100° C. outside of the glove box for 1 h under Ar, cooled down to room temperature and concentrated under reduced pressure. The resulting residue was purified by preparative HPLC (column: Phenomenex Synergi C18 150*25 mm*10 μm; mobile phase: A: 0.225% formic acid in water, B: MeCN; B %: 31%-61%, 10 min) and lyophilized directly to afford the product 3-(5-(difluoromethyl)-1,3,4-thiadiazol-2-yl)-N-(1-methylcyclopropyl)-8-(2-oxa-7-azaspiro[3.5]nonan-7-yl)imidazo[1,2-a]pyridine-6-sulfonamide (7.35 mg, 13.67 μmol, 19.64% yield, 94.99% purity) as a light yellow solid.

RT 0.918 min (Method 1); m/z 511.0 (M+H)⁺ (ESI⁺); ¹H NMR (CDCl₃, 400 MHz) 9.88 (d, J=1.6 Hz, 1H), 8.18 (s, 1H), 6.95-7.24 (m, 2H), 5.06 (s, 1H), 4.54 (s, 4H), 3.49-3.60 (m, 4H), 2.11-2.21 (m, 4H), 1.35 (s, 3H), 0.87-0.95 (m, 2H), 0.55-0.62 (m, 2H).

### Preparation of Intermediate 43.1

### 8-chloro-3-(5-(difluoromethyl)-1,3,4-thiadiazol-2-yl)imidazo[1,2-a]pyridine-6-sulfonyl bromide

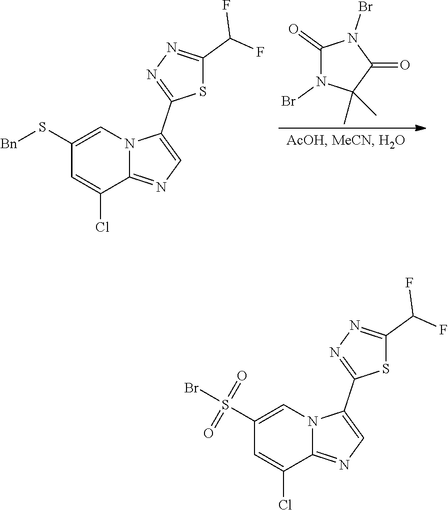

Three batches were run in parallel and mixed for the work-up.

To a mixture of 2-(6-(benzylthio)-8-chloroimidazo[1,2-a]pyridin-3-yl)-5-(difluoromethyl)-1,3,4-thiadiazole (1 g, 2.45 mmol), AcOH (293.74 mg, 4.89 mmol, 279.75 μL), H₂O (88.12 mg, 4.89 mmol, 88.12 μL) in MeCN (10 mL) was added 1,3-dibromo-5,5-dimethylimidazolidine-2,4-dione (2.80 g, 9.78 mmol) at 0° C. and the mixture was stirred at 0° C. for 0.5 h. The 3 batches were combined. The resulting mixture was quenched by ice/water (90 mL) and extracted with DCM (90 mL, 3×). The combined organic layer was washed with brine (50 mL, 2×), dried over anhydrous Na₂SO₄, filtered and concentrated under reduced pressure. The resulting residue was purified by column chromatography (SiO₂, Petroleum ether/Ethyl acetate (10% DCM additive)=5/1 to 1/1) to afford the product 8-chloro-3-[5-(difluoromethyl)-1,3,4-thiadiazol-2-yl]imidazo[1,2-a]pyridine-6-sulfonyl bromide (3.15 g, 7.33 mmol, 99.73%) a yellow solid.

RT 0.770 min (Method 1); m/z 430.7 (M+H)⁺ (ESI⁺)

### Preparation of Compound 43

### 8-chloro-3-(5-(difluoromethyl)-1,3,4-thiadiazol-2-yl)-N-(1-(fluoromethyl)cyclopropyl)imidazo[1,2-a]pyridine-6-sulfonamide

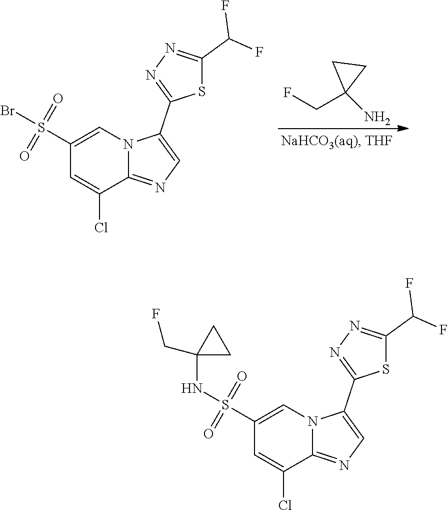 [SMILES: O=S(=O)(Br)c1cc(Cl)c2ncc(-c3nnc(C(F)F)s3)n2c1 ; NC1(CF)CC1 ; O=S(=O)(NC1(CF)CC1)c1cc(Cl)c2ncc(-c3nnc(C(F)F)s3)n2c1]

Three batches were run in parallel and mixed for the work-up.

To a mixture of 1-(fluoromethyl)cyclopropan-1-amine (705.83 mg, 5.62 mmol, HCl salt) in saturated NaHCO₃ (7 mL) was added 8-chloro-3-(5-(difluoromethyl)-1,3,4-thiadiazol-2-yl)imidazo[1,2-a]pyridine-6-sulfonyl bromide (1.05 g, 2.44 mmol) in THE (5 mL) and the mixture was stirred at 15° C. for 1 h. The 3 batches were combined. The resulting mixture was diluted with H₂O (60 mL) and extracted with EtOAc (60 mL, 2×). Then, the combined organic layer was washed with brine (30 mL, 2×), dried over anhydrous Na₂SO₄ filtered and concentrated under reduced pressure. The resulting residue was purified by column chromatography (SiO₂, Petroleum ether/Ethyl acetate=5/1 to 2/1) to give 1.7 g of an impure product. 50 mg were purified by preparative HPLC (column: Phenomenex luna C18 150*25 mm*10 μm; mobile phase: A: 0.225% formic acid in water; B: MeCN; B %: 41%-61%, 10 min) and lyophilized directly to give the product 8-chloro-3-(5-(difluoromethyl)-1,3,4-thiadiazol-2-yl)-N-(1-(fluoromethyl)cyclopropyl)imidazo[1,2-a]pyridine-6-sulfonamide (17.49 mg, 39.95 μmol, 8.06% yield) as white solid.

RT 0.784 min (Method 1); m/z 438.1 (M+H)⁺ (ESI⁺); ¹H NMR (DMSO-d₆, 400 MHz): 9.97 (d, J=1.6 Hz, 1H), 9.08-8.90 (m, 1H), 8.79 (s, 1H), 7.94 (d, J=1.6 Hz, 1H), 7.71 (t, J=53.2 Hz, 1H), 4.24 (d, J=48.4 Hz, 1H), 0.92-0.84 (m, 2H), 0.83-0.76 (m, 2H).

### Preparation of Example 44

### 4-(3-(5-(difluoromethyl)-1,3,4-thiadiazol-2-yl)-6-(N-(1-(fluoromethyl)cyclopropyl)sulfamoyl)imidazo [1,2-a]pyridin-8-yl)-N,N-dimethylpiperazine-1-carboxamide

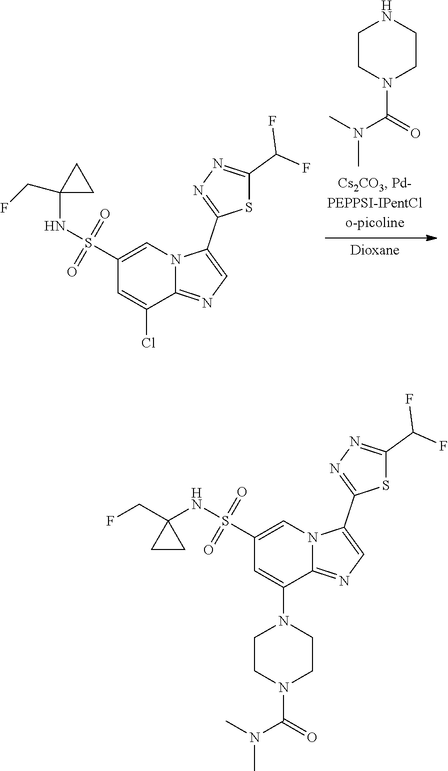 [SMILES: O=S(=O)(NC1(CF)CC1)c1cc(Cl)c2ncc(-c3nnc(C(F)F)s3)n2c1 ; CN(C)C(=O)N1CCNCC1 ; CN(C)C(=O)N1CCN(c2cc(S(=O)(=O)NC3(CF)CC3)cn3c(-c4nnc(C(F)F)s4)cnc23)CC1]

In a glove box, to a solution of N,N-dimethylpiperazine-1-carboxamide (14.36 mg, 91.36 μmol) in dioxane (0.5 mL) was added 8-chloro-3-(5-(difluoromethyl)-1,3,4-thiadiazol-2-yl)-N-(1-(fluoromethyl)cyclopropyl)imidazo[1,2-a]pyridine-6-sulfonamide (20 mg, 45.68 μmol), Cs₂CO₃ (29.77 mg, 91.36 μmol) and Pd-PEPPSI-IPentCl o-picoline (1.97 mg, 2.28 μmol). The mixture was stirred at 100° C. outside of the glove box for 2 h under Ar. The reaction mixture was cooled down to room temperature and concentrated under reduced pressure. The residue was diluted with MeOH (3 mL), filtered and the filtrate was purified by preparative HPLC (column: Unisil 3-100 C18 Ultra 150*50 mm*3 μm; mobile phase: A: 0.225% formic acid in water, B: MeCN; B %: 29%-59%, 7 min) and lyophilized directly to afford the product 4-(3-(5-(difluoromethyl)-1,3,4-thiadiazol-2-yl)-6-(N-(1-(fluoromethyl)cyclopropyl)sulfamoyl)imidazo[1,2-a]pyridin-8-yl)-N,N-dimethylpiperazine-1-carboxamide (5.73 mg, 10.16 μmol, 22.23% yield, 99% purity) as a light yellow solid.

RT 0.887 min (Method 1); m/z 559.1 (M+H)⁺ (ESI⁺); ¹H NMR (DMSO-d₆, 400 MHz): 9.65 (s, 1H), 8.67-8.82 (m, 1H), 8.62 (s, 1H), 7.71 (t, J=53.2 Hz, 1H), 7.02 (s, 1H), 4.23 (d, J=48.8 Hz, 2H), 3.63 (br, 4H), 3.36 (br, 4H), 2.81 (s, 6H), 0.81-0.88 (m, 2H), 0.74-0.81 (m, 2H).

### Preparation of Example 45

### 3-(5-(difluoromethyl)-1,3,4-thiadiazol-2-yl)-8-(4-((dimethyl(oxo)-16-sulfaneylidene)amino)piperidin-1-yl)-N-(1-methylcyclopropyl)imidazo[1,2-a]pyridine-6-sulfonamide

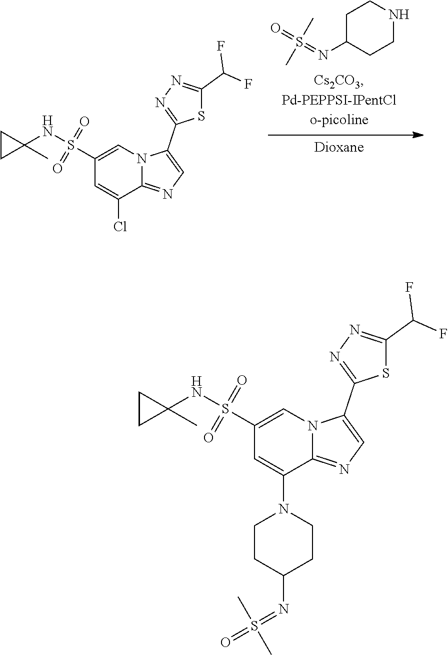

In a glove box, to the mixture of 8-chloro-3-(5-(difluoromethyl)-1,3,4-thiadiazol-2-yl)-N-(1-methylcyclopropyl)imidazo[1,2-a]pyridine-6-sulfonamide (15 mg, 35.73 μmol) in dioxane was added dimethyl(piperidin-4-ylimino)-λ⁶-sulfanone (13.35 mg, 53.59 μmol, 2 HCl salt), Pd-PEPPSI-IPentCl o-picoline (1.74 mg, 1.79 μmol) and Cs₂CO₃ (46.56 mg, 142.91 μmol). The mixture was stirred at 100° C. outside of the glove box for 2 h under Ar, cooled down to room temperature, diluted with H₂O (30 mL) and extracted with EtOAc (30 mL, 3×). The combined organic layer was washed with brine (30 mL), dried over Na₂SO₄, filtered and concentrated under reduced pressure. The resulting residue was purified by preparative HPLC (column: Phenomenex Synergi C18 150*25 mm*10 μm; mobile phase: A: 0.225% formic acid in water, B: MeCN; B %: 29%-59%, 10 min) and lyophilized directly to afford the product 3-(5-(difluoromethyl)-1,3,4-thiadiazol-2-yl)-8-(4-((dimethyl(oxo)-16-sulfaneylidene)amino)piperidin-1-yl)-N-(1-methylcyclopropyl)imidazo [1,2-a]pyridine-6-sulfonamide (6.53 mg, 11.67 μmol, 32.66% yield, 100% purity) as a yellow solid.

RT 0.904 min (Method 1); m/z 560.1 (M+H)⁺ (ESI⁺); ¹H NMR (DMSO-d₆, 400 MHz) 9.61 (d, J=1.2 Hz, 1H), 8.58 (s, 1H), 8.39 (s, 1H), 7.69 (t, J=53.2 Hz, 1H), 6.97 (s, 1H), 4.11-4.23 (m, 2H), 3.44-3.52 (m, 1H), 3.30 (s, 6H), 3.15-3.23 (m, 2H), 1.81-1.96 (m, 2H), 1.56-1.71 (m, 2H), 1.13 (s, 3H), 0.68-0.74 (m, 2H), 0.41-0.46 (m, 2H).

### Preparation of Example 46

### 3-(5-(difluoromethyl)-1,3,4-thiadiazol-2-yl)-8-(6-(hydroxymethyl)-3-azabicyclo[3.1.1]heptan-3-yl)-N-(1-methylcyclopropyl)imidazo[1,2-a]pyridine-6-sulfonamide

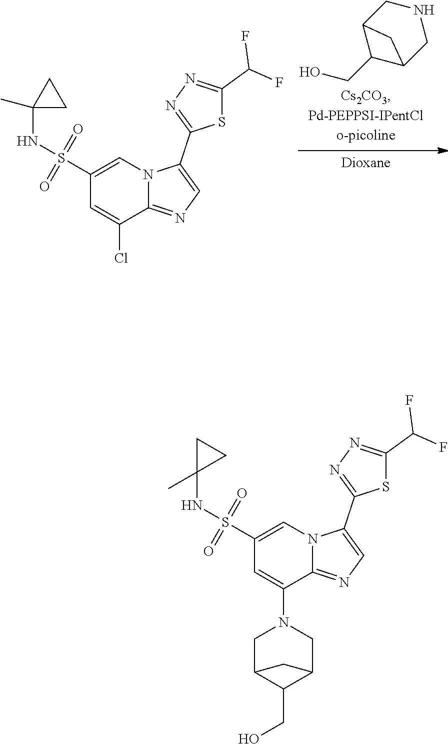

In a glove box, to a mixture of 8-chloro-3-(5-(difluoromethyl)-1,3,4-thiadiazol-2-yl)-N-(1-methylcyclopropyl)imidazo[1,2-a]pyridine-6-sulfonamide (50 mg, 119.09 μmol) in dioxane (0.6 mL) was added (3-azabicyclo[3.1.1]heptan-6-yl)methanol (29.23 mg, 178.64 μmol, HCl salt), Pd-PEPPSI-IPentCl o-picoline (5.79 mg, 5.95 μmol) and Cs₂CO₃ (116.40 mg, 357.27 μmol). The mixture was stirred at 100° C. outside of the glove box for 2 h under Ar. The reaction mixture was cooled down to room temperature, diluted with H₂O (30 mL) and extracted with EtOAc (30 mL, 3×). The combined organic layer was washed with brine (30 mL), dried over Na₂SO₄, filtered and concentrated under reduced pressure. The resulting residue was purified by preparative HPLC (column: Phenomenex Synergi C18 150*25 mm*10 μm; mobile phase: A: 0.225% formic acid in water, B: MeCN; B %: 29%-59%, 10 min) and lyophilized directly to afford the product 3-(5-(difluoromethyl)-1,3,4-thiadiazol-2-yl)-8-(6-(hydroxymethyl)-3-azabicyclo[3.1.1]heptan-3-yl)-N-(1-methylcyclopropyl)imidazo[1,2-a]pyridine-6-sulfonamide (6.53 mg, 11.67 μmol, 32.66% yield, 100% purity) as a yellow solid

RT 1.010 min (Method 1); m/z 511.1 (M+H)⁺ (ESI⁺); ¹H NMR (DMSO-d₆, 400 MHz) 9.45-9.52 (m, 1H), 8.52-8.57 (m, 1H), 8.30-8.43 (m, 1H), 7.54-7.87 (m, 1H), 6.61-6.67 (m, 1H), 4.18-4.24 (m, 1H), 3.96-4.13 (m, 3H), 3.67-3.71 (m, 1H), 3.39-3.43 (m, 1H), 2.35-2.45 (m, 3H), 1.82-2.08 (m, 1H), 1.38-1.51 (m, 1H), 1.16 (d, J=2.4 Hz, 3H), 0.69-0.76 (m, 2H), 0.41-0.47 (m, 2H).

### Preparation of Example 47

### 3-(5-(difluoromethyl)-1,3,4-thiadiazol-2-yl)-N-(1-(fluoromethyl)cyclopropyl)-8-(2-oxa-7-azaspiro[3.5]nonan-7-yl)imidazo[1,2-a]pyridine-6-sulfonamide

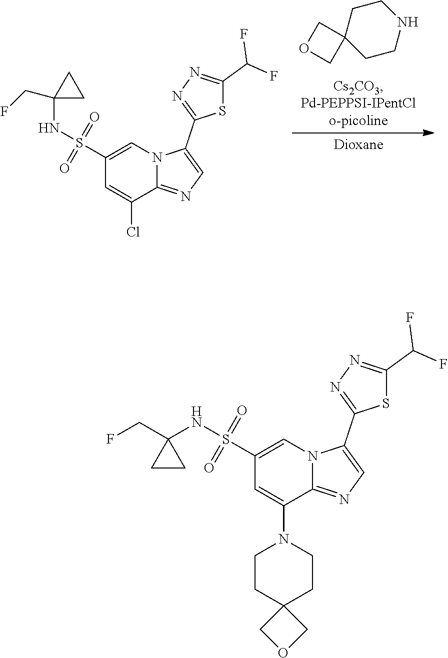

To a solution of 8-chloro-3-(5-(difluoromethyl)-1,3,4-thiadiazol-2-yl)-N-(1-(fluoromethyl)cyclopropyl)imidazo[1,2-a]pyridine-6-sulfonamide (30 mg, 68.52 μmol) in dioxane (0.5 mL) was added 2-oxa-7-azaspiro[3.5]nonane (24.79 mg, 102.78 μmol, TFA salt) and Cs₂CO₃ (66.97 mg, 205.55 μmol). In a glove box, Pd-PEPPSI-IPentCl o-picoline (2.95 mg, 3.43 μmol) was added. The mixture was stirred at 100° C. outside of the glove box for 1 h under Ar, then cooled down to room temperature and concentrated under reduced pressure. The resulting residue was purified by preparative HPLC (column: Phenomenex luna C18 150*25 mm*10 μm; mobile phase: A: 0.225% formic acid in water, B: MeCN; B %: 38%-68%, 10 min) and lyophilized directly to afford the product 3-(5-(difluoromethyl)-1,3,4-thiadiazol-2-yl)-N-(1-(fluoromethyl)cyclopropyl)-8-(2-oxa-7-azaspiro[3.5]nonan-7-yl)imidazo[1,2-a]pyridine-6-sulfonamide (4.9 mg, 9.27 μmol, 13.53% yield, 100% purity) as a yellow solid.

RT 0.855 min (Method 1); m/z 529.1 (M+H)⁺ (ESI⁺); ¹H NMR (DMSO-d₆, 400 MHz) 9.88 (d, J=1.6 Hz, 1H), 8.18 (s, 1H), 6.93-7.24 (m, 2H), 5.46 (s, 1H), 4.54 (s, 4H), 4.25 (d, J=48.8 Hz, 2H), 3.51-3.61 (m, 4H), 2.10-2.18 (m, 4H), 1.09-1.18 (m, 2H), 0.83-0.88 (m, 2H)

### Preparation of Compound 48

### 3-(5-(difluoromethyl)-1,3,4-thiadiazol-2-yl)-N-(1-methylcyclopropyl)-8-(4-(methylthio)piperidin-1-yl)imidazo[1,2-a]pyridine-6-sulfonamide

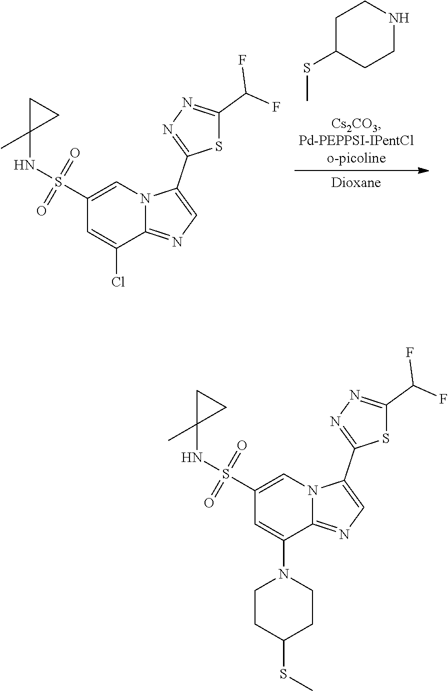

To a solution of 4-(methylthio)piperidine (53.92 mg, 321.54 μmol, HCl salt) in dioxane (1 mL) was added 8-chloro-3-(5-(difluoromethyl)-1,3,4-thiadiazol-2-yl)-N-(1-methylcyclopropyl)imidazo[1,2-a]pyridine-6-sulfonamide (90 mg, 214.36 μmol) and Cs₂CO₃ (209.53 mg, 643.08 μmol). In a glove box, Pd-PEPPSI-IPentCl o-picoline (9.22 mg, 10.72 μmol) was added. The mixture was stirred at 100° C. outside of the glove box for 2 h under Ar₂, then, cooled to room temperature, diluted with H₂O (30 mL) and extracted with EtOAc (30 mL, 3×). The combined organic layer was washed with brine (30 mL), dried over Na₂SO₄, filtered and concentrated under reduced pressure. The resulting residue was purified by preparative HPLC (column: Phenomenex luna C18 150*25 mm*10 μm; mobile phase: A: 0.225% formic acid in water, B: MeCN; B %: 51%-81%, 10 min) and lyophilized directly to afford the product 3-(5-(difluoromethyl)-1,3,4-thiadiazol-2-yl)-N-(1-methylcyclopropyl)-8-(4-(methylthio)piperidin-1-yl)imidazo[1,2-a]pyridine-6-sulfonamide (25 mg, 48.07 μmol, 22.42% yield, 98.95% purity) as a yellow solid

RT 0.634 min (Method 3); m/z 515.3 (M+H)⁺ (ESI⁺); ¹H NMR (CDCl₃, 400 MHz) 9.87 (d, J=1.6 Hz, 1H), 8.17 (s, 1H), 6.95-7.24 (m, 2H), 5.05 (s, 1H), 4.18-4.28 (m, 2H), 3.10-3.20 (m, 2H), 2.79-2.92 (m, 1H), 2.15-2.25 (m, 5H), 1.85-1.98 (m, 2H), 1.36 (s, 3H), 0.87-0.95 (m, 2H), 0.56-0.62 (m, 2H)

### Preparation of Example 49

### 3-(5-(difluoromethyl)-1,3,4-thiadiazol-2-yl)-N-(1-methylcyclopropyl)-8-(4-(S-methylsulfonimidoyl) piperidin-1-yl)imidazo[1,2-a]pyridine-6-sulfonamide

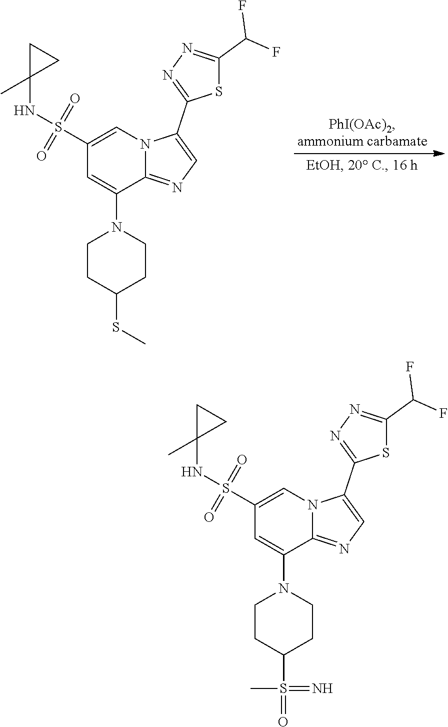 [SMILES: CSC1CCN(c2cc(S(=O)(=O)NC3(C)CC3)cn3c(-c4nnc(C(F)F)s4)cnc23)CC1 ; CC1(NS(=O)(=O)c2cc(N3CCC(S(C)(=N)=O)CC3)c3ncc(-c4nnc(C(F)F)s4)n3c2)CC1]

To a mixture of 3-[5-(difluoromethyl)-1,3,4-thiadiazol-2-yl]-N-(1-methylcyclopropyl)-8-(4-methylsulfanyl-1-piperidyl)imidazo[1,2-a]pyridine-6-sulfonamide (20 mg, 38.86 μmol) in EtOH (0.5 mL) was added PhI(OAc)₂ (50.07 mg, 155.45 μmol) and ammonium carbamate (6.07 mg, 77.73 μmol). The mixture was stirred at 20° C. for 16 h, then diluted with H₂O (20 mL) and extracted with EtOAc (20 mL, 2×). The combined organic layer was washed with brine (20 mL, 2×), dried over Na₂SO₄ and concentrated under reduced pressure. The resulting residue was purified by preparative HPLC (column: Phenomenex luna C18 150*25 mm*10 μm; mobile phase: A: 0.225% formic acid in water, B: MeCN; B %: 24%-54%, 10 min) and lyophilized directly to afford the product 3-(5-(difluoromethyl)-1,3,4-thiadiazol-2-yl)-N-(1-methylcyclopropyl)-8-(4-(S-methylsulfonimidoyl)piperidin-1-yl)imidazo[1,2-a]pyridine-6-sulfonamide (12.51 mg, 22.93 μmol, 58.99% yield, 100% purity) as a yellow solid.

RT 0.780 min (Method 1); m/z 546.1 (M+H)⁺ (ESI⁺); ¹H NMR (CDCl₃, 400 MHz) 9.92 (d, J=1.2 Hz, 1H), 8.19 (s, 1H), 6.96-7.26 (m, 2H), 5.30 (s, 1H), 4.61 (t, J=12.4 Hz, 2H), 3.14-3.30 (m, 1H), 2.94-3.06 (m, 5H), 2.33-2.50 (m, 2H), 2.08-2.22 (m, 2H), 1.37 (s, 3H), 0.89-0.99 (m, 2H), 0.56-0.65 (m, 2H)

### Preparation of Example 50

### 3-(5-(difluoromethyl)-1,3,4-thiadiazol-2-yl)-N-(1-methylcyclopropyl)-8-(4-(methylsulfonyl)piperidin-1-yl)imidazo[1,2-a]pyridine-6-sulfonamide

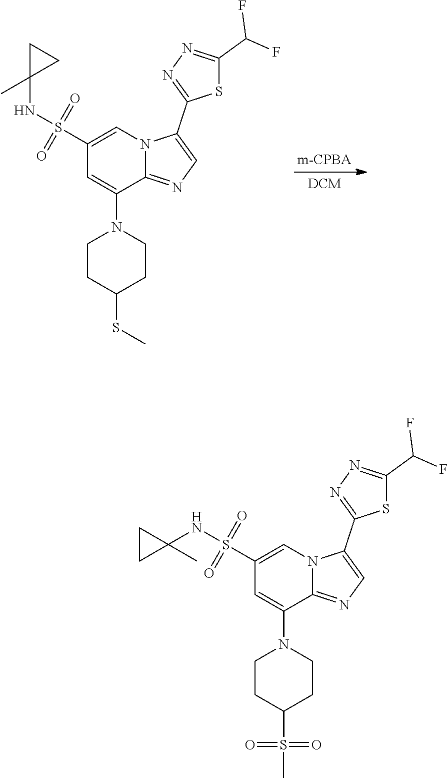 [SMILES: CSC1CCN(c2cc(S(=O)(=O)NC3(C)CC3)cn3c(-c4nnc(C(F)F)s4)cnc23)CC1 ; CC1(NS(=O)(=O)c2cc(N3CCC(S(C)(=O)=O)CC3)c3ncc(-c4nnc(C(F)F)s4)n3c2)CC1]

To a solution of 3-(5-(difluoromethyl)-1,3,4-thiadiazol-2-yl)-N-(1-methylcyclopropyl)-8-(4-(methylthio)piperidin-1-yl)imidazo[1,2-a]pyridine-6-sulfonamide (20 mg, 38.86 μmol) in DCM (0.5 mL) was added m-CPBA (15.39 mg, 75.78 μmol, 85% purity) and the mixture was stirred at 0° C. for 1 h. The reaction mixture was diluted with NaS₂O₃ (aq., sat., 20 mL) and extracted with EtOAc (20 mL, 2×). The combined organic layer was washed with brine (20 mL, 2×), dried over aqueous Na₂SO₄ and concentrated under reduced pressure. The resulting residue was purified by preparative HPLC (Column: Waters Xbridge 150*25 mm*5 μm; mobile phase: A: 10 mM aqueous solution of NH₄HCO₃ in water, B: MeCN; B %: 37%-67%, 10 min) and lyophilized directly to give the product 3-(5-(difluoromethyl)-1,3,4-thiadiazol-2-yl)-N-(1-methylcyclopropyl)-8-(4-(methylsulfonyl)piperidin-1-yl)imidazo[1,2-a]pyridine-6-sulfonamide (2.13 mg, 3.78 μmol, 9.73% yield, 97% purity) as a light yellow solid.

RT 0.679 min (Method 3); m/z 547.2 (M+H)⁺ (ESI⁺); ¹H NMR (CDCl₃, 400 MHz) 9.93 (d, J=1.6 Hz, 1H), 8.20 (s, 1H), 6.96-7.26 (m, 2H), 5.09 (s, 1H), 4.53-4.65 (m, 2H), 3.07-3.18 (m, 1H), 2.96-3.06 (m, 2H), 2.93 (s, 3H), 2.35-2.46 (m, 2H), 2.11-2.26 (m, 2H), 1.39 (s, 3H), 0.90-0.97 (m, 2H), 0.57-0.65 (m, 2H).

### Preparation of Example 51

### 3-(5-(difluoromethyl)-1,3,4-thiadiazol-2-yl)-N-(1-methylcyclopropyl)-8-(4-(methylsulfinyl)piperidin-1-yl)imidazo[1,2-a]pyridine-6-sulfonamide

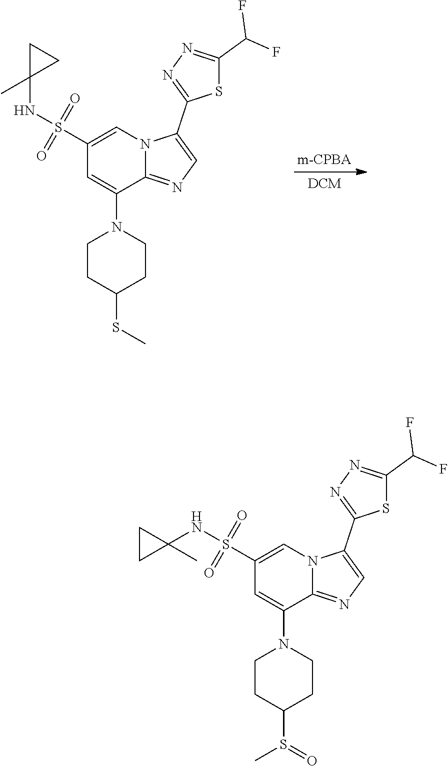 [SMILES: CSC1CCN(c2cc(S(=O)(=O)NC3(C)CC3)cn3c(-c4nnc(C(F)F)s4)cnc23)CC1 ; CS(=O)C1CCN(c2cc(S(=O)(=O)NC3(C)CC3)cn3c(-c4nnc(C(F)F)s4)cnc23)CC1]

To a solution of 3-(5-(difluoromethyl)-1,3,4-thiadiazol-2-yl)-N-(1-methylcyclopropyl)-8-(4-(methylthio)piperidin-1-yl)imidazo[1,2-a]pyridine-6-sulfonamide (30 mg, 58.29 μmol) in DCM (0.5 mL) was added m-CPBA (11.24 mg, 55.38 μmol, 85% purity) and the mixture was stirred at 0° C. for 1 h. The reaction mixture was quenched with Na₂S₂O₃ (aq., sat., 20 mL) and extracted with EtOAc (20 mL*2). The combined organic layer was washed with brine (20 mL*2), dried over anhydrous Na₂SO₄ and concentrated under reduced pressure to give a residue, which was purified by preparative HPLC (column: Phenomenex luna C18 150*25 mm*10 um; mobile phase: A: 0.225% formic acid in water, B: MeCN; B %: 33%-63%, 10 min) and lyophilized to give the product 3-(5-(difluoromethyl)-1,3,4-thiadiazol-2-yl)-N-(1-methylcyclopropyl)-8-(4-(methylsulfinyl)piperidin-1-yl)imidazo[1,2-a]pyridine-6-sulfonamide (5.96 mg, 11.10 μmol, 19.04% yield, 98.822% purity) as a yellow solid.

RT 0.679 min (Method 1); m/z 531.1 (M+H)⁺ (ESI⁺); ¹H NMR (CDCl₃, 400 MHz) 8.89 (d, J=1.2 Hz, 1H), 8.17 (s, 1H), 7.23-6.96 (m, 2H), 5.34 (s, 1H), 4.52-4.44 (m, 2H), 3.13-3.06 (m, 1H), 2.86-2.84 (m, 2H), 2.64 (s, 3H), 2.34-2.33 (m, 1H), 2.10-2.02 (m, 3H), 1.36 (s, 3H), 0.93-0.90 (m, 2H), 0.60-0.57 (m, 2H)

Compounds listed in the table below were prepared according to the corresponding general procedures or, when stated in a similar way to the related example, and starting from the corresponding intermediates or examples. It is noted that the skilled person is capable to select the correct intermediate and reaction conditions for obtaining any of the compounds listed in the Table below (or in any other Table in this application referring to the compound synthesis by reference to any general procedure).

| Cpd | | Yield | | | |
|---|---|---|---|---|---|
| number | Structure | (%) | Procedure | LC/MS | 1H NMR |
| 52 | 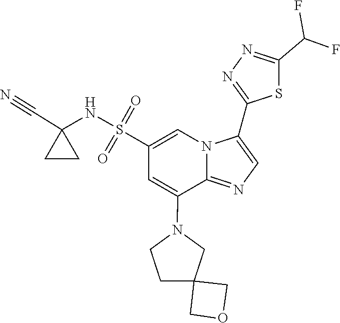 [SMILES: N#CC1(NS(=O)(=O)c2cc(N3CCC4(COC4)C3)c3ncc(-c4nnc(C(F)F)s4)n3c2)CC1] | 7.56 | Procedure 1 | RT 0.774 min (method 1); m/z 508.1 (M + H)⁺ (ESI⁺) | ¹H NMR (DMSO-d₆, 400 MHz): 9.37-9.54 (m, 1H), 8.55 (s, 1H), 8.33-8.45 (m, 1H), 7.53- 7.93 (m, 1H), 6.52 (s, 1H), 4.64 (d, J = 5.6 Hz, 2H), 4.57 (d, J = 6.0 Hz, 2H), 4.12 (s, 2H), 3.75-3.80 (m, 2H), 1.10- 1.45 (m, 6H). |
| 53 | 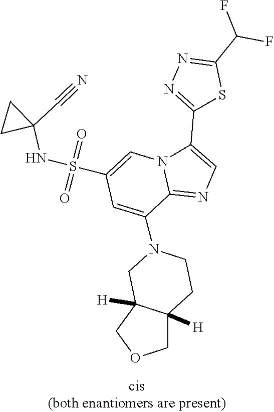 [SMILES: N#CC1(NS(=O)(=O)c2cc(N3CC[C@@H]4COC[C@@H]4C3)c3ncc(-c4nnc(C(F)F)s4)n3c2)CC1] | 3.33 | Procedure 1 | RT 0.742 min (method 1); m/z 522.1 (M + H)⁺ (ESI⁺) | ¹H NMR (DMSO-d₆, 400 MHz): 9.61 (s, 1H), 8.58 (s, 1H), 8.31-8.40 (m, 1H), 7.71 (t, J = 53.2 Hz, 1H), 6.94 (s, 1H), 3.79-3.83 (m, 4H), 3.64- 3.69 (m, 4H), 1.72-1.99 (m, 4H), 1.25-1.34 (m, 4H). |
| 54 | 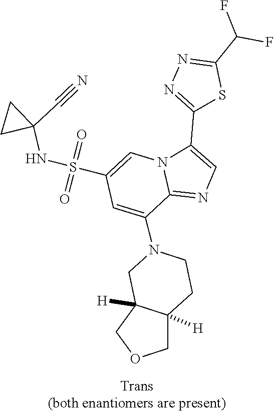 [SMILES: N#CC1(NS(=O)(=O)c2cc(N3CC[C@H]4COC[C@@H]4C3)c3ncc(-c4nnc(C(F)F)s4)n3c2)CC1] | 15.43 | Procedure 1 | RT 0.886 min (method 1); m/z 522.1 (M + H)⁺ (ESI⁺) | ¹H NMR (DMSO-d₆, 400 MHz): 9.66 (s, 1H), 8.62 (s, 1H), 7.71 (t, J = 53.2 Hz, 1H), 6.95 (d, J = 1.2 Hz, 1H), 3.85- 3.93 (m, 1H), 3.77-3.84 (m, 3H), 3.62-3.75 (m, 3H), 3.49- 3.60 (m, 1H), 2.55-2.59 (m, 1H), 2.42-2.46 (m, 1H), 1.91- 2.01 (m, 1H), 1.69-1.81 (m, 1H), 1.30-1.48 (m, 4H). |
| 55 | 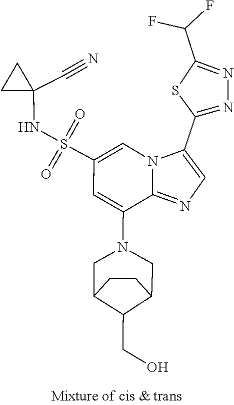 [SMILES: N#CC1(NS(=O)(=O)c2cc(N3CC4CCC(C3)C4CO)c3ncc(-c4nnc(C(F)F)s4)n3c2)CC1] | 9 | Procedure 1 | RT 0.974 min (method 1); m/z 536.1 (M + H)⁺ (ESI⁺) | ¹H NMR (DMSO-d₆, 400 MHz): 9.64 (s, 1H), 8.56-8.62 (m, 1H), 7.69 (t, J = 53.2 Hz, 1H), 6.83-6.89 (m, 1H), 3.77- 4.56 (m, 3H), 3.19-3.26 (m, 2H), 2.89-2.98 (m, 1H), 2.28- 2.34 (m, 2H), 1.84-1.90 (m, 1H), 1.65-1.82 (m, 4H), 1.35- 1.44 (m, 2H), 1.26-1.34 (m, 2H) |
| 56 | 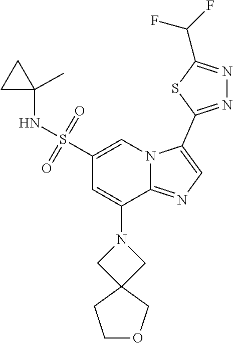 [SMILES: CC1(NS(=O)(=O)c2cc(N3CC4(CCOC4)C3)c3ncc(-c4nnc(C(F)F)s4)n3c2)CC1] | 11.69 | Procedure 1 | RT 0.922 min (method 1); m/z 497.2 (M + H)⁺ (ESI⁺) | ¹H NMR (DMSO-d₆, 400 MHz): 8.87 (s, 1H), 8.60 (s, 1H), 8.25 (s, 1H), 7.63 (t, J = 53.20 Hz, 1H), 6.20 (s, 1H), 4.26 (s, 4H), 3.88 (s, 2H), 3.77 (t, J = 7.2 Hz, 2H), 2.23 (t, J = 6.8 Hz, 2H), 1.16 (s, 3H), 0.66- 0.73 (m, 2H), 0.38-0.47 (m, 2H) |
| 57 | 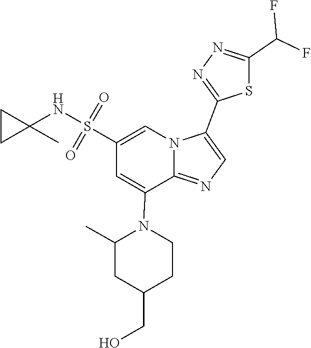 [SMILES: CC1CC(CO)CCN1c1cc(S(=O)(=O)NC2(C)CC2)cn2c(-c3nnc(C(F)F)s3)cnc12] | 1.03 | Procedure 1 | RT 0.839 min (method 1); m/z 513.2 (M + H)⁺ (ESI⁺) | ¹H NMR (CDCl₃, 400 MHz): 10.00 (s, 1H), 8.16 (s, 1H), 6.87-7.16 (m, 2H), 5.25 (br, 1H), 3.73-3.88 (m, 1H), 3.50- 3.64 (m, 2H), 3.05 (br, 1H), 1.95-2.02 (m, 1H), 1.80-1.89 (m, 2H), 1.62- 1.71 (m, 2H), 1.26-1.29 (m, 3H), 1.09 (d, J = 6.00 Hz, 3H), 0.79-0.87 (m, 2H), 0.47-0.56 (m, 2H) |
| 58 | 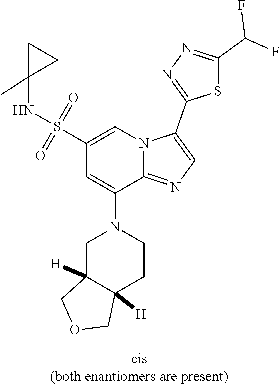 [SMILES: CC1(NS(=O)(=O)c2cc(N3CC[C@@H]4COC[C@@H]4C3)c3ncc(-c4nnc(C(F)F)s4)n3c2)CC1] | 2.77 | Procedure 1 | RT 0.920 min (method 1); m/z 511.2 (M + H)⁺ (ESI⁺) | H NMR (CDCl₃, 400 MHz): 9.84 (d, J = 1.6 Hz, 1H), 8.17 (s, 1H), 7.11 (t, J = 53.2 Hz, 1H), 6.94 (d, J = 1.2 Hz, 1H), 5.04 (s, 1H), 3.95- 4.02 (m, 2H), 3.84-3.94 (m, 2H), 3.72-3.82 (m, 2H), 3.63- 3.71 (m, 1H), 3.49-3.59 (m, 1H), 2.64-2.73 (m, 1H), 2.48- 2.57 (m, 1H), 2.03-2.15 (m, 1H), 1.89-1.98 (m, 1H), 1.38 (s, 3H), 0.90- 0.98 (m, 2H), 0.57-0.63 (m, 2H) |
| 59 | 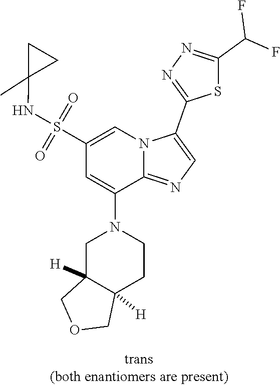 [SMILES: CC1(NS(=O)(=O)c2cc(N3CC[C@H]4COC[C@@H]4C3)c3ncc(-c4nnc(C(F)F)s4)n3c2)CC1] | 1.61 | Procedure 1 | RT 0.919 min (method 1); m/z 511.2 (M + H)⁺ (ESI⁺) | ¹H NMR (DMSO-d₆, 400 MHz): 9.59 (d, J = 1.2 Hz, 1H), 8.59 (s, 1H), 8.39 (s, 1H), 7.53-7.86 (m, 1H), 6.96 (s, 1H), 3.78- 3.88 (m, 4H), 3.61-3.71 (m, 3H), 3.47-3.56 (m, 1H), 2.38- 2.48 (m, 2H), 1.86-2.01 (m, 1H), 1.66-1.80 (m, 1H), 1.14 (s, 3H), 0.67- 0.79 (m, 2H), 0.41-0.50 (m, 2H) |
| 60 | 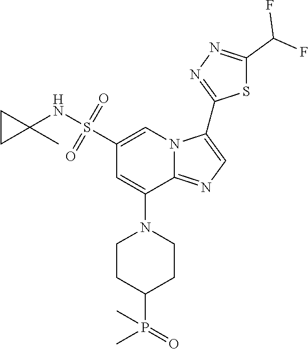 [SMILES: CC1(NS(=O)(=O)c2cc(N3CCC(P(C)(C)=O)CC3)c3ncc(-c4nnc(C(F)F)s4)n3c2)CC1] | 20.86 | Procedure 1 | RT 0.922 min (method 1); m/z 545.1 (M + H)⁺ (ESI⁺) | ¹H NMR (DMSO-d₆, 400 MHz): 9.63 (s, 1H), 8.60 (s, 1H), 8.39 (s, 1H), 7.69 (t, J = 53.2 Hz, 1H), 6.99 (s, 1H), 4.44-4.56 (m, 2H), 2.88-2.97 (m, 2H), 1.88- 2.00 (m, 3H), 1.64-1.74 (m, 2H), 1.39 (s, 3H), 1.36 (s, 3H), 1.13 (s, 3H), 0.68-0.73 (m, 2H), 0.40- 0.47 (m, 2H) |
| 61 | 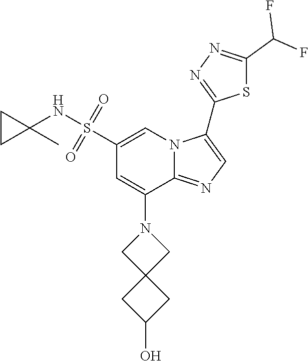 [SMILES: CC1(NS(=O)(=O)c2cc(N3CC4(CC(O)C4)C3)c3ncc(-c4nnc(C(F)F)s4)n3c2)CC1] | 2.01 | Procedure 1 | RT 0.858 min (method 1); m/z 497.1 (M + H)⁺ (ESI⁺) | ¹H NMR (CDCl₃, 400 MHz): 9.61 (d, J = 1.6 Hz, 1H), 8.10 (s, 1H), 7.01 (t, J = 53.2 Hz, 1H), 6.38 (d, J = 1.2 Hz, 1H), 4.99 (s, 1H), 4.38 (s, 2H), 4.32 (s, 3H), 2.65-2.74 (m, 2H), 2.18- 2.27 (m, 2H), 1.35 (s, 3H), 0.89-0.94 (m, 2H), 0.54-0.61 (m, 2H) |
| 62 | 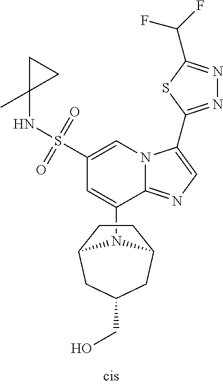 [SMILES: CC1(NS(=O)(=O)c2cc(N3[C@@H]4CC[C@H]3C[C@H](CO)C4)c3ncc(-c4nnc(C(F)F)s4)n3c2)CC1] | 14.9 | Procedure 1 | RT 1.023 min (method 1); m/z 525.1 (M + H)⁺ (ESI⁺) | ¹H NMR (DMSO-d₆, 400 MHz): 9.48 (d, J = 1.2 Hz, 1H), 8.56 (s, 1H), 8.38 (s, 1H), 7.70 (t, J = 53.2 Hz, 1H), 6.85 (s, 1H), 5.06-5.30 (m, 2H), 4.38 (t, J = 5.2 Hz, 1H), 3.06-3.12 (m, 2H), 1.99-2.13 (m, 3H), 1.78- 1.89 (m, 2H), 1.37-1.61 (m, 4H), 1.06-1.16 (m, 3H), 0.65- 0.76 (m, 2H), 0.35-0.48 (m, 2H) |
| 63 | 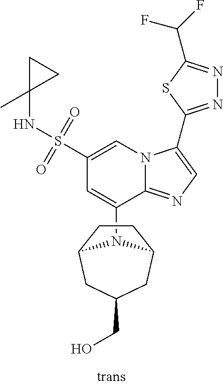 [SMILES: CC1(NS(=O)(=O)c2cc(N3[C@@H]4CC[C@H]3C[C@@H](CO)C4)c3ncc(-c4nnc(C(F)F)s4)n3c2)CC1] | 19.02 | Procedure 1 | RT 0.889 min (method 1); m/z 525.2 (M + H)⁺ (ESI⁺) | ¹H NMR (DMSO-d₆, 400 MHz): 9.48 (d, J = 1.2 Hz, 1H), 8.57 (s, 1H), 8.38 (s, 1H), 7.55-7.87 (m, 1H), 6.87 (s, 1H), 5.01- 5.19 (m, 2H), 4.63 (t, J = 5.6 Hz, 1H), 3.41- 3.45 (m, 2H), 2.04-2.14 (m, 4H), 1.77-1.83 (m, 2H), 1.57- 1.64 (m, 1H), 1.44-1.53 (m, 2H), 1.13 (s, 3H), 0.65-0.76 (m, 2H), 0.40- 0.47 (m, 2H) |
| 64 | 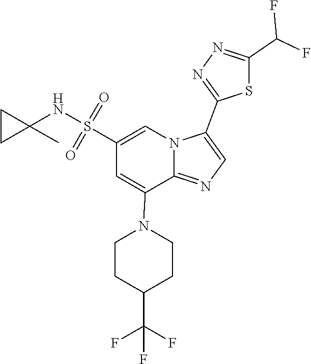 [SMILES: CC1(NS(=O)(=O)c2cc(N3CCC(C(F)(F)F)CC3)c3ncc(-c4nnc(C(F)F)s4)n3c2)CC1] | 21.49 | Procedure 1 | RT 0.981 min (method 1); m/z 537.1 (M + H)⁺ (ESI⁺) | ¹H NMR (DMSO-d₆, 400 MHz): 9.64 (d, J = 1.6 Hz, 1H), 8.61 (s, 1H), 8.42 (s, 1H), 7.70 (t, J = 53.2 Hz, 1H), 7.01 (d, J = 1.2 Hz, 1H), 4.42- 4.54 (m, 2H), 2.92-3.03 (m, 2H), 2.60-2.67 (m, 1H), 1.94- 2.03 (m, 2H), 1.58-1.75 (m, 2H), 1.12 (s, 3H), 0.66-0.74 (m, 2H), 0.40- 0.48 (m, 2H) |
| 65 | 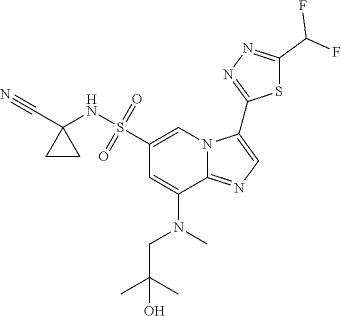 [SMILES: CN(CC(C)(C)O)c1cc(S(=O)(=O)NC2(C#N)CC2)cn2c(-c3nnc(C(F)F)s3)cnc12] | 17.07 | Procedure 1 | RT 0.854 min (method 1); m/z 498.1 (M + H)⁺ (ESI⁺) | ¹H NMR (DMSO-d₆, 400 MHz): 9.57 (d, J = 1.6 Hz, 1H), 8.60 (s, 1H), 7.70 (t, J = 53.2 Hz, 1H), 6.73 (d, J = 1.2 Hz, 1H), 4.62 (s, 1H), 4.37 (s, 2H), 3.27 (s, 3H), 1.39-1.47 (m, 2H), 1.32- 1.38 (m, 2H), 1.04 (s, 6H) |
| 66 | 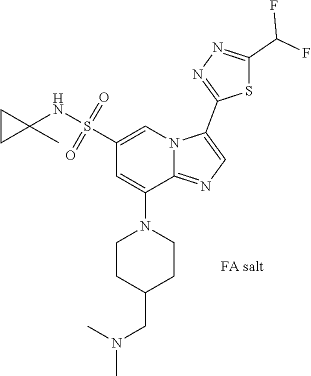 [SMILES: CN(C)CC1CCN(c2cc(S(=O)(=O)NC3(C)CC3)cn3c(-c4nnc(C(F)F)s4)cnc23)CC1] | 27.93 | Procedure 1 | RT 0.791 min (method 1); m/z 526.2 (M + H)⁺ (ESI⁺) | ¹H NMR (CDCl₃, 400 MHz): 9.86 (d, J = 1.6 Hz, 1H), 8.41 (s, 1H), 8.16 (s, 1H), 6.94-7.24 (m, 2H), 5.37 (s, 1H), 4.32- 4.41 (m, 2H), 2.90-3.02 (m, 2H), 2.62-2.66 (m, 2H), 2.59 (s, 6H), 2.02- 2.07 (m, 3H), 1.57-1.68 (m, 2H), 1.34 (s, 3H), 0.88-0.95 (m, 2H), 0.54- 0.60 (m, 2H) |
| 67 | 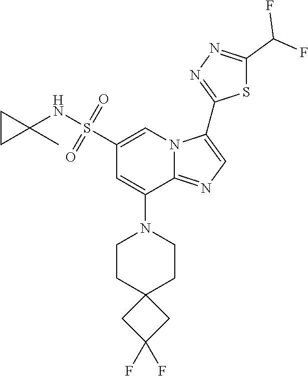 [SMILES: CC1(NS(=O)(=O)c2cc(N3CCC4(CC3)CC(F)(F)C4)c3ncc(-c4nnc(C(F)F)s4)n3c2)CC1] | 12.55 | Procedure 1 | RT 0.995 min (method 1); m/z 544.9 (M + H)⁺ (ESI⁺) | ¹H NMR (CDCl₃, 400 MHz): 9.88 (d, J = 1.6 Hz, 1H), 8.17 (s, 1H), 6.95-7.24 (m, 2H), 5.06 (s, 1H), 3.53- 3.62 (m, 4H), 2.46 (t, J = 12.4 Hz, 4H), 1.91- 2.01 (m, 4H), 1.36 (s, 3H), 0.90-0.95 (m, 2H), 0.56-0.63 (m, 2H) |
| 68 | 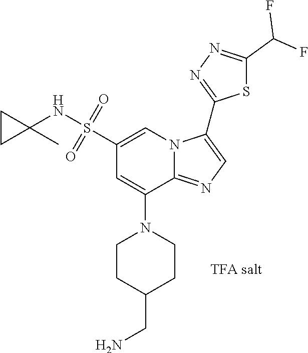 [SMILES: CC1(NS(=O)(=O)c2cc(N3CCC(CN)CC3)c3ncc(-c4nnc(C(F)F)s4)n3c2)CC1] | 15.87 | Procedure 1 followed by procedure 2 (yield after 2 steps) | RT 0.776 min (method 1); m/z 498.2 (M + H)⁺ (ESI⁺) | ¹H NMR (DMSO-d₆, 400 MHz): 9.63 (d, J = 1.2 Hz, 1H), 8.60 (s, 1H), 8.39 (s, 1H), 7.55-7.87 (m, 3H), 6.99 (d, J = 1.6 Hz, 1H), 4.34-4.47 (m, 2H), 2.88- 2.97 (m, 2H), 2.77-2.85 (m, 2H), 1.80-1.94 (m, 3H), 1.37- 1.50 (m, 2H), 1.13 (s, 3H), 0.65-0.74 (m, 2H), 0.40-0.49 (m, 2H) |
| 69 | 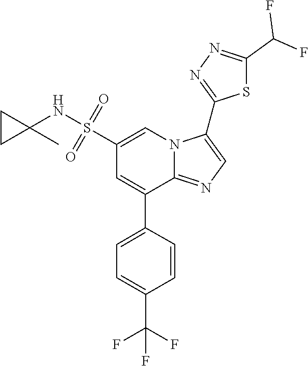 [SMILES: CC1(NS(=O)(=O)c2cc(-c3ccc(C(F)(F)F)cc3)c3ncc(-c4nnc(C(F)F)s4)n3c2)CC1] | 10.27 | Procedure 6 | RT 0.833 min (method 1); m/z 530.1 (M + H)⁺ (ESI⁺) | ¹H NMR (CDCl₃, 400 MHz): 10.37 (s, 1H), 8.36 (s, 1H), 8.14 (d, J = 8.0 Hz, 2H), 7.95 (s, 1H), 7.85 (d, J = 8.4 Hz, 2H), 6.99-7.27 (m, 1H), 5.21 (s, 1H), 1.43 (s, 3H), 0.90- 0.98 (m, 2H), 0.58-0.70 (m, 2H) |

Compound 55 appears to include two isomers, according to the following structures:

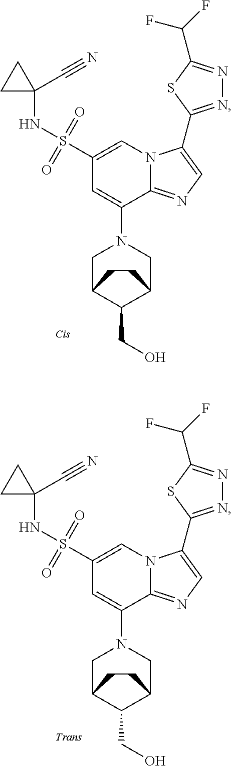 [SMILES: N#CC1(NS(=O)(=O)c2cc(N3C[C@H]4CC[C@@H](C3)[C@@H]4CO)c3ncc(-c4nnc(C(F)F)s4)n3c2)CC1 ; N#CC1(NS(=O)(=O)c2cc(N3C[C@H]4CC[C@@H](C3)[C@H]4CO)c3ncc(-c4nnc(C(F)F)s4)n3c2)CC1]

### Preparation of Example 70a

### Tert-butyl 4-(3-(5-(difluoromethyl)-1,3,4-thiadiazol-2-yl)-6-(N-(1-methylcyclopropyl)sulfamoyl)imidazo[1,2-a]pyridin-8-yl)-3,6-dihydropyridine-1(2H)-carboxylate

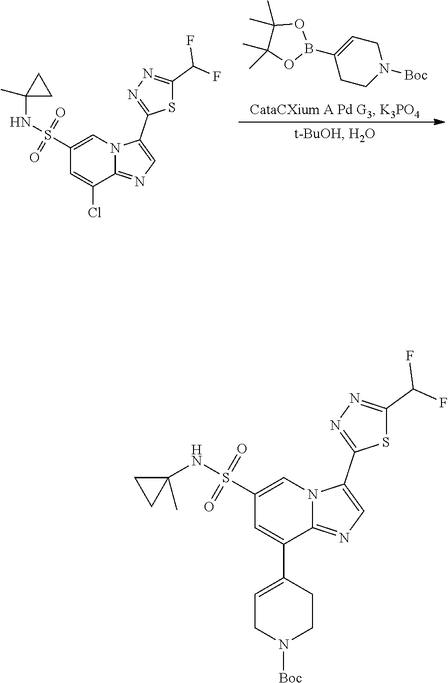

A mixture of tert-butyl 4-(4,4,5,5-tetramethyl-1,3,2-dioxaborolan-2-yl)-3,6-dihydropyridine-1(2H)-carboxylate (220.94 mg, 714.53 μmol), 8-chloro-3-(5-(difluoromethyl)-1,3,4-thiadiazol-2-yl)-N-(1-methylcyclopropyl)imidazo[1,2-a]pyridine-6-sulfonamide (200 mg, 476.35 μmol), CataCXium A-Pd-G3 and K₃P04 aqueous solution (1.5 M, 317.57 μL) in n-BuOH (5 mL) was degassed, purged with N₂ (3×) and stirred at 60° C. for 16 h under N₂ atmosphere. The mixture was cooled to 20° C., poured into water (20 mL) and filtered. The resulting solid was triturated with PE:EA=3:1 at 20° C. for 10 min. After filtration, the solid was dried under vacuum to give 170 mg of a crude product. 10 mg of this crude product were purified by preparative HPLC (Column: Unisil 3-100 C18 Ultra 150*50 mm*3 μm; mobile phase: A: 0.225% formic acid in water, B: MeCN; B %: 54%-84%, 7 min) and lyophilized directly to give the product tert-butyl 4-(3-(5-(difluoromethyl)-1,3,4-thiadiazol-2-yl)-6-(N-(1-methylcyclopropyl)sulfamoyl)imidazo[1,2-a]pyridin-8-yl)-3,6-dihydropyridine-1(2H)-carboxylate (6.05 mg, 10.68 μmol, 38.08% yield, 100% purity) as a yellow solid

RT 0.991 min (Method 1); m/z 567.2 (M+H)⁺ (ES⁺); ¹H NMR (CDCl₃, 400 MHz) 10.22 (d, J=1.6 Hz, 1H), 8.28 (s, 1H), 7.67 (d, J=1.6 Hz, 1H), 6.95-7.25 (m, 2H), 5.13 (s, 1H), 4.20-4.30 (m, 2H), 3.75 (t, J=5.6 Hz, 2H), 2.70-2.80 (m, 2H), 1.52 (s, 9H), 1.37 (s, 3H), 0.86-0.95 (m, 2H), 0.58-0.64 (m, 2H)

### Preparation of Example 70

### 3-(5-(difluoromethyl)-1,3,4-thiadiazol-2-yl)-N-(1-methylcyclopropyl)-8-(1,2,3,6-tetrahydropyridin-4-yl)imidazo[1,2-a]pyridine-6-sulfonamide hydrogen chloride

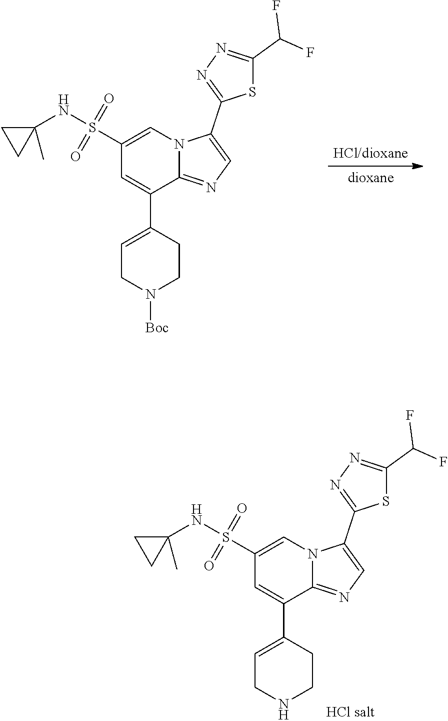 [SMILES: CC1(NS(=O)(=O)c2cc(C3=CCN(C(=O)OC(C)(C)C)CC3)c3ncc(-c4nnc(C(F)F)s4)n3c2)CC1 ; CC1(NS(=O)(=O)c2cc(C3=CCNCC3)c3ncc(-c4nnc(C(F)F)s4)n3c2)CC1]

To a solution of tert-butyl 4-(3-(5-(difluoromethyl)-1,3,4-thiadiazol-2-yl)-6-(N-(1-methylcyclopropyl)sulfamoyl)imidazo[1,2-a]pyridin-8-yl)-3,6-dihydropyridine-1(2H)-carboxylate (150 mg, 264.72 μmol) in DCM (2 mL) was added HCl/dioxane (4 M, 2 mL) and the mixture was stirred at 20° C. for 1 h. The reaction mixture was concentrated under reduced pressure to give 90 mg of crude product. 15 mg of this crude product were purified by preparative HPLC (column: Phenomenex luna C18 150*25 mm*10 μm; mobile phase: A: 0.225% formic acid in water, B: MeCN; B %: 10%-40%, 10 min) and lyophilized directly to give the product 3-(5-(difluoromethyl)-1,3,4-thiadiazol-2-yl)-N-(1-methylcyclopropyl)-8-(1,2,3,6-tetrahydropyridin-4-yl)imidazo[1,2-a]pyridine-6-sulfonamide hydrogen chloride (3.69 mg, 7.91 μmol, 18.94% yield, 100% purity, HCl salt) as a yellow solid

RT 0.767 min (method 1); m/z 467.1 (M+H)⁺ (ES⁺); ¹H NMR (CDCl₃, 400 MHz) 10.23 (d, J=1.6 Hz, 1H), 8.27 (s, 1H), 7.70 (d, J=1.6 Hz, 1H), 6.98-7.27 (m, 2H), 5.09-5.21 (m, 1H), 3.72-3.78 (m, 2H), 3.27 (t, J=5.6 Hz, 2H), 2.69-2.78 (m, 2H), 1.38 (s, 3H), 0.90-0.94 (m, 2H), 0.59-0.65 (m, 2H).

### Preparation of Example 71

### 4-(3-(5-(difluoromethyl)-1,3,4-thiadiazol-2-yl)-6-(N-(1-methylcyclopropyl)sulfamoyl)imidazo[1,2-a]pyridin-8-yl)-N,N-dimethyl-3,6-dihydropyridine-1(2H)-carboxamide

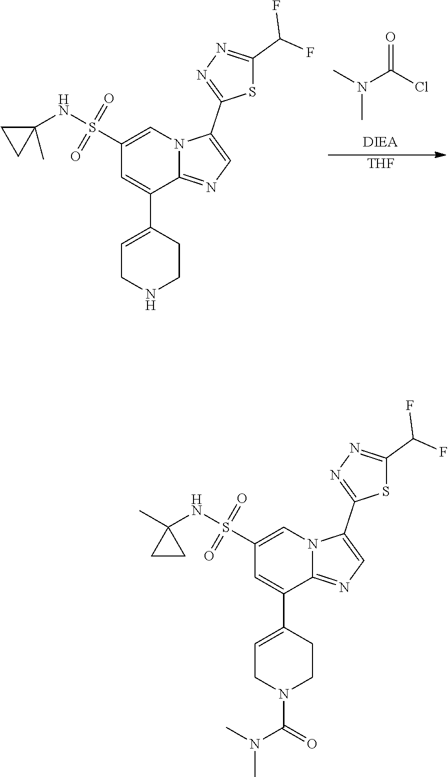

To a solution of 3-(5-(difluoromethyl)-1,3,4-thiadiazol-2-yl)-N-(1-methylcyclopropyl)-8-(1,2,3,6-tetrahydropyridin-4-yl)imidazo[1,2-a]pyridine-6-sulfonamide (20 mg, 42.87 μmol) in THE (0.5 mL) was added DIEA (11.08 mg, 85.74 μmol, 14.93 μL) and dimethylcarbamic chloride (3.69 mg, 34.30 μmol, 3.15 μL) and the mixture was stirred at 0° C. for 2 h. The reaction mixture was concentrated under reduced pressure. The resulting residue was purified by preparative HPLC (column: Unisil 3-100 C18 Ultra 150*50 mm*3 μm; mobile phase: A: 0.225% formic acid in water, B: MeCN; B %: 44%-74%, 7 min) and lyophilized directly to give the product 4-(3-(5-(difluoromethyl)-1,3,4-thiadiazol-2-yl)-6-(N-(1-methylcyclopropyl)sulfamoyl)imidazo[1,2-a]pyridin-8-yl)-N,N-dimethyl-3,6-dihydropyridine-1(2H)-carboxamide (3.82 mg, 7.11 μmol, 16.57% yield) as a white solid.

RT 0.620 min (method 3); m/z 538.2 (M+H)⁺ (ES⁺); ¹H NMR (DMSO-d₆, 400 MHz) 9.99 (d, J=1.6 Hz, 1H), 8.75 (s, 1H), 8.45-8.55 (m, 1H), 7.56-7.87 (m, 2H), 7.40 (s, 1H), 4.00 (d, J=1.6 Hz, 2H), 3.42-3.46 (m, 2H), 2.81 (s, 6H), 2.70-2.80 (m, 3H), 1.14 (s, 3H), 0.68-0.75 (m, 2H), 0.43-0.50 (m, 2H)

### Preparation of Example 72

### 3-(5-(difluoromethyl)-1,3,4-thiadiazol-2-yl)-8-(3-hydroxy-3-methylbut-1-yn-1-yl)-N-(1-methylcyclopropyl)imidazo[1,2-a]pyridine-6-sulfonamide

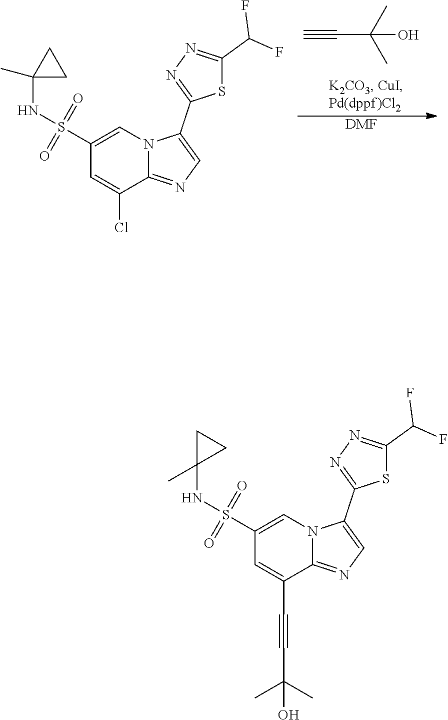

To a solution of 8-chloro-3-(5-(difluoromethyl)-1,3,4-thiadiazol-2-yl)-N-(1-methylcyclopropyl)imidazo[1,2-a]pyridine-6-sulfonamide (30 mg, 71.45 μmol) in DMF (1 mL) was added K₂CO₃ (39.50 mg, 285.81 μmol), Pd(dppf)Cl₂ (5.23 mg, 7.15 μmol) and CuI (1.36 mg, 7.15 μmol). The reaction mixture was degassed and purged with N₂ (3×), followed by addition of 2-methylbut-3-yn-2-ol (6.61 mg, 78.60 μmol, 7.68 μL). The reaction mixture was then stirred at 100° C. for 2 hr under N₂ atmosphere followed by concentration under reduced pressure. The resulting residue was diluted with MeOH (3 mL), filtered and the filtrate was purified by preparative HPLC (column: Phenomenex Synergi C18 150*25 mm*10 μm; mobile phase: A: 0.225% formic acid in water, B: MeCN; B %: 34%-64%, 10 min) and lyophilized directly to give the product 3-(5-(difluoromethyl)-1,3,4-thiadiazol-2-yl)-8-(3-hydroxy-3-methylbut-1-yn-1-yl)-N-(1-methylcyclopropyl)imidazo[1,2-a]pyridine-6-sulfonamide (7.42 mg, 15.87 μmol, 22.21% yield, 100% purity) as off-white solid.

RT 0.980 min (Method 1); m/z 468.1 (M+H)⁺ (ES⁺); ¹H NMR (400 MHz, DMSO-d₆) 9.99 (d, J=1.6 Hz, 1H), 8.75 (s, 1H), 8.37-8.62 (m, 1H), 7.53-7.86 (m, 2H), 5.73 (s, 1H), 1.55 (s, 6H), 1.15 (s, 3H), 0.67-0.74 (m, 2H), 0.41-0.49 (m, 2H).

### Preparation of Example 73

### 3-(5-(difluoromethyl)-1,3,4-thiadiazol-2-yl)-8-(3-hydroxy-3-methylbutyl)-N-(1-methylcyclopropyl)imidazo[1,2-a]pyridine-6-sulfonamide

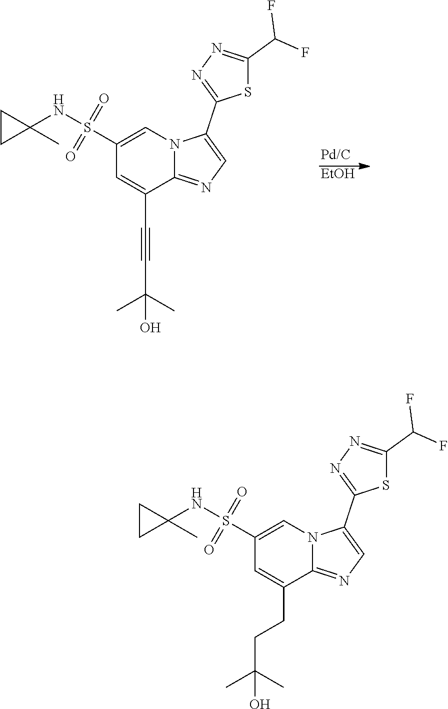 [SMILES: "EtOH" ; CC(C)(O)C#Cc1cc(S(=O)(=O)NC2(C)CC2)cn2c(-c3nnc(C(F)F)s3)cnc12 ; CC(C)(O)CCc1cc(S(=O)(=O)NC2(C)CC2)cn2c(-c3nnc(C(F)F)s3)cnc12]

To a solution of 3-(5-(difluoromethyl)-1,3,4-thiadiazol-2-yl)-8-(3-hydroxy-3-methylbut-1-yn-1-yl)-N-(1-methylcyclopropyl)imidazo[1,2-a]pyridine-6-sulfonamide (10 mg, 21.39 μmol) in EtOH (1 mL) was added Pd/C (5 mg, 10% purity) under N₂. The suspension was degassed under vacuum; purged with H₂ (3×) and stirred under H₂ (15 psi) at 20° C. for 2 h. The reaction mixture was filtered and the filtrate was concentrated under reduced pressure. The resulting residue was purified by preparative HPLC (column: Waters Xbridge 150*25 mm*5 μm; mobile phase: A: 0.225% formic acid in water, B: MeCN; B %: 16%-46%, 10 min) and lyophilized directly to give the product 3-(5-(difluoromethyl)-1,3,4-thiadiazol-2-yl)-8-(3-hydroxy-3-methylbutyl)-N-(1-methylcyclopropyl)imidazo[1,2-a]pyridine-6-sulfonamide (0.89 mg, 1.89 μmol, 8.82% yield, 100% purity) as a light yellow solid.

RT 0.889 min (method 1); m/z 472.0 (M+H)⁺ (ES⁺); ¹H NMR (400 MHz, CDCl₃) 10.16 (d, J=1.6 Hz, 1H), 8.27 (s, 1H), 7.63 (s, 1H), 7.105 (t, J=53.2 Hz, 1H), 5.13 (s, 1H), 3.20-3.30 (m, 2H), 1.97-2.03 (m, 2H), 1.36 (s, 9H), 0.86-0.92 (m, 2H), 0.55-0.63 (m, 2H).

### Preparation of Example 74

### 3-(5-(difluoromethyl)-1,3,4-thiadiazol-2-yl)-N-(1-(fluoromethyl)cyclopropyl)imidazo[1,2-a]pyridine-6-sulfonamide

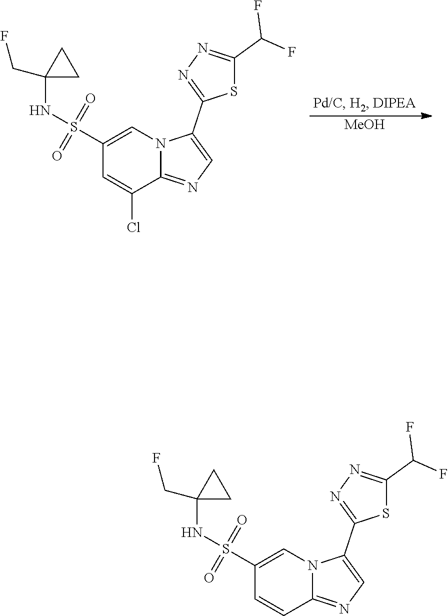 [SMILES: "MeOH" ; O=S(=O)(NC1(CF)CC1)c1cc(Cl)c2ncc(-c3nnc(C(F)F)s3)n2c1 ; O=S(=O)(NC1(CF)CC1)c1ccc2ncc(-c3nnc(C(F)F)s3)n2c1]

To a solution of 8-chloro-3-(5-(difluoromethyl)-1,3,4-thiadiazol-2-yl)-N-(1-(fluoromethyl)cyclopropyl)imidazo[1,2-a]pyridine-6-sulfonamide (50 mg, 114.20 μmol) in MeOH (2 mL) was added Pd/C (25 mg, 114.20 μmol, 10% purity), DIEA (44.28 mg, 342.59 μmol, 59.67 μL) under N₂. The suspension was degassed under vacuum, purged with H₂ (3×) and stirred under H₂ (15 psi) at 20° C. for 24 h. The reaction mixture was filtered and the filtrate was concentrated under reduced pressure. The resulting residue was purified by preparative HPLC (column: Phenomenex luna C18 150*25 mm*10 μm; mobile phase: mobile phase: A: 0.225% formic acid in water; B: MeCN; B %: 29%-59%, 10 min) and lyophilized directly to give the product 3-(5-(difluoromethyl)-1,3,4-thiadiazol-2-yl)-N-(1-(fluoromethyl)cyclopropyl)imidazo[1,2-a]pyridine-6-sulfonamide (1.31 mg, 3.25 μmol, 2.84% yield, 100% purity) as a yellow solid.

RT 0.557 min (Method 3); m/z 403.9 (M+H)⁺ (ES⁺); ¹H NMR (DMSO-d₆, 400 MHz): 10.03 (s, 1H), 8.92 (br, 1H), 8.75 (s, 1H), 8.05 (d, J=9.2 Hz, 1H), 7.85-7.56 (m, 2H), 4.235 (d, J=48.8 Hz, 2H), 0.76-0.85 (m, 4H).

### Preparation of Intermediate 75.1

### Ethyl 8-chloro-6-(chlorosulfonyl)imidazo[1,2-a]pyridine-3-carboxylate

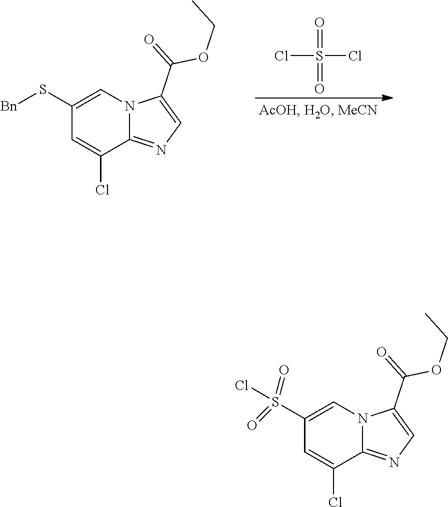 [SMILES: CCOC(=O)c1cnc2c(Cl)cc(SCc3ccccc3)cn12 ; O=S(=O)(Cl)Cl ; CCOC(=O)c1cnc2c(Cl)cc(S(=O)(=O)Cl)cn12]

To a mixture of ethyl 6-(benzylthio)-8-chloroimidazo[1,2-a]pyridine-3-carboxylate (500 mg, 1.44 mmol), AcOH (329.80 μL, 5.77 mmol), H₂O (103.89 μL, 5.77 mmol) in MeCN (5 mL) was added sulfuryl dichloride (504.46 μL, 5.05 mmol) at 0° C. The mixture was stirred at 0° C. for 0.5 h, then, diluted with DCM (30 mL), washed by ice-H₂O (30 mL), brine (30 mL), dried over Na₂SO₄, filtered and concentrated under reduced pressure at 20° C. to give the product ethyl 8-chloro-6-(chlorosulfonyl)imidazo[1,2-a]pyridine-3-carboxylate (450 mg, 1.39 mmol, 96.59% yield) as yellow oil. The crude product was used into next step directly without purification.

RT 0.724 min (Method 1); m/z 322.7 (M+H)⁺ (ESI⁺)

### Preparation of Example 75

### Ethyl 8-chloro-6-(N-(1-cyanocyclopropyl)sulfamoyl)imidazo[1,2-a]pyridine-3-carboxylate

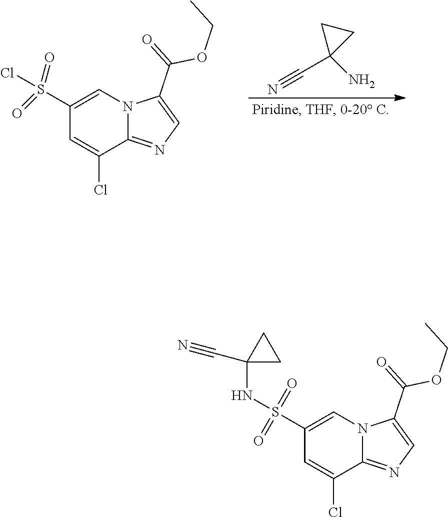 [SMILES: CCOC(=O)c1cnc2c(Cl)cc(S(=O)(=O)Cl)cn12 ; N#CC1(N)CC1 ; CCOC(=O)c1cnc2c(Cl)cc(S(=O)(=O)NC3(C#N)CC3)cn12]

To a mixture of 1-aminocyclopropane-1-carbonitrile (165.11 mg, 1.39 mmol, HCl salt) in Pyridine (561.99 μL, 6.96 mmol) was added drop-wise ethyl 8-chloro-6-(chlorosulfonyl)imidazo[1,2-a]pyridine-3-carboxylate (450 mg, 1.39 mmol) in THF (5 mL) at 0° C. The mixture was stirred at 20° C. for 16 h, then cooled to room temperature, quenched by H₂O (30 mL) and extracted with EtOAc (30 mL, 3×). The combined organic layer was washed with brine (30 mL), dried over Na₂SO₄, filtered and concentrated under reduced pressure. The residue was purified by flash silica gel chromatography (ISCO®; 12 g SepaFlash® Silica Flash Column, Eluent of 30-60% Ethyl acetate/Petroleum ether @75 mL/min) and concentrated to give the product ethyl 8-chloro-6-(N-(1-cyanocyclopropyl)sulfamoyl)imidazo[1,2-a]pyridine-3-carboxylate (0.3 g, 813.46 μmol, 58.42% yield) as a white solid.

RT 0.834 min (method 1); m/z 369.0 (M+H)⁺ (ESI⁺); ¹H NMR (DMSO-d₆, 400 MHz): 9.71 (d, J=1.6 Hz, 1H), 9.67-9.39 (m, 1H), 8.51 (s, 1H), 7.95 (d, J=1.6 Hz, 1H), 4.48-4.39 (m, 2H), 1.54-1.46 (m, 2H), 1.44-4.34 (m, 5H).

### Preparation of Example 76

### Ethyl 8-chloro-6-(N-(1-methylcyclopropyl)sulfamoyl)imidazo[1,2-a]pyridine-3-carboxylate

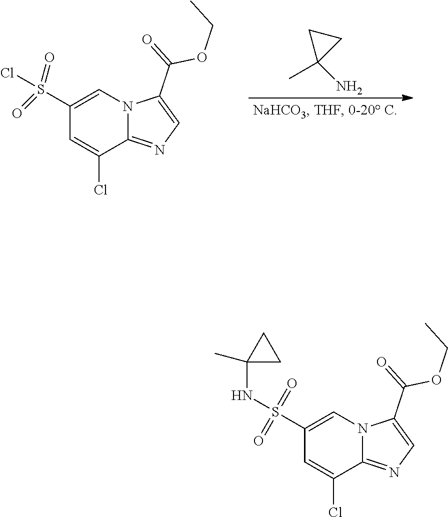

To a solution of 1-methylcyclopropan-1-amine (1.20 g, 11.14 mmol, HCl salt) in NaHCO₃ (aq., sat., 30 mL) was added drop-wise ethyl 8-chloro-6-(chlorosulfonyl)imidazo[1,2-a]pyridine-3-carboxylate (1.8 g, 5.57 mmol) in THE (15 mL) at 0° C. The mixture was stirred at 15° C. for 2 h then, quenched by H₂O (50 mL) and extracted with EtOAc (50 mL, 3×). The combined organic layer was washed with brine (50 mL), dried over Na₂SO₄, filtered and concentrated under reduced pressure. The resulting residue was purified by flash silica gel chromatography (ISCO®; 40 g SepaFlash® Silica Flash Column, Eluent of 25-40% Ethyl acetate/Petroleum ether @100 mL/min) and concentrated to give the product ethyl 8-chloro-6-(N-(1-methylcyclopropyl)sulfamoyl)imidazo[1,2-a]pyridine-3-carboxylate (0.6 g, 1.68 mmol, 30.10% yield) as a white solid.

¹H NMR (DMSO-d₆, 400 MHz): 9.62 (d, J=1.6 Hz, 1H), 8.52 (s, 1H), 8.46 (s, 1H), 7.85-7.95 (m, 1H), 4.41 (q, 7.2 Hz, 2H), 1.38 (t, J=7.2 Hz, 3H), 1.16 (s, 3H), 0.65-0.74 (m, 2H), 0.40-0.52 (m, 2H).

### Preparation of Intermediate 77.1

### 8-chloro-6-(N-(1-methylcyclopropyl)sulfamoyl)imidazo[1,2-a]pyridine-3-carboxylic acid

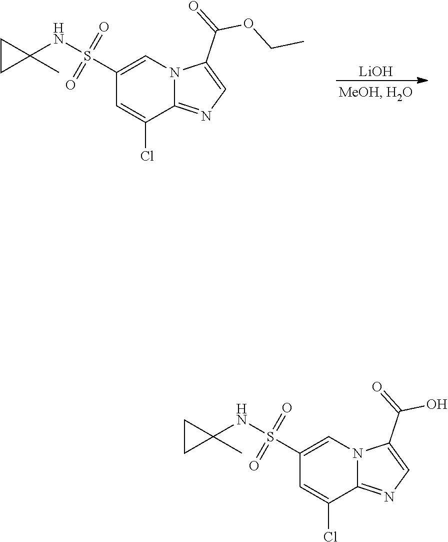 [SMILES: CCOC(=O)c1cnc2c(Cl)cc(S(=O)(=O)NC3(C)CC3)cn12 ; "LiOH" ; CC1(NS(=O)(=O)c2cc(Cl)c3ncc(C(=O)O)n3c2)CC1]

To a solution of ethyl 8-chloro-6-(N-(1-methylcyclopropyl)sulfamoyl)imidazo[1,2-a]pyridine-3-carboxylate (400 mg, 1.12 mmol) in MeOH (2 mL) and H₂O (2 mL) was added LiOH (160.63 mg, 6.71 mmol) in one portion. The reaction mixture was stirred at 60° C. for 2 h. The reaction mixture was acidified by 1M hydrochloric acid aqueous solution until pH=4. The white precipitate was filtered, collected and dried under reduced pressure to give the product 8-chloro-6-(N-(1-methylcyclopropyl)sulfamoyl)imidazo[1,2-a]pyridine-3-carboxylic acid (280 mg, 679.28 μmol, 60.76% yield, 80% purity) as a yellow solid.

RT 0.559 min (method 1); m/z 330.1 (M+H)⁺ (ESI⁺); ¹H NMR (DMSO-d₆, 400 MHz): 9.66 (s, 1H), 8.50 (s, 1H), 8.40 (s, 1H), 7.88 (s, 1H), 1.15 (s, 3H), 0.64-0.73 (m, 2H), 0.41-0.50 (m, 2H).

### Preparation of Example 77

### 8-chloro-N-isobutyl-6-(N-(1-methylcyclopropyl)sulfamoyl)imidazo[1,2-a]pyridine-3-carboxamide

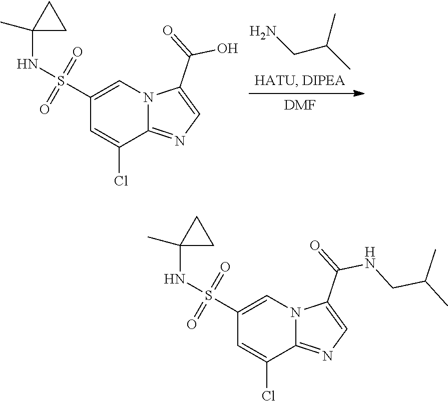

To a mixture of 8-chloro-6-(N-(1-methylcyclopropyl)sulfamoyl)imidazo[1,2-a]pyridine-3-carboxylic acid (40 mg, 121.30 μmol) in DMF (1 mL) was added HATU (55.35 mg, 145.56 μmol) and DIPEA (31.35 mg, 242.60 μmol). The mixture was stirred at 15° C. for 10 min. Then, 2-methylpropan-1-amine (17.74 mg, 242.60 μmol) was added and the mixture was stirred at 15° C. for 16 h. The reaction mixture was cooled to room temperature, quenched by H₂O (30 mL) and extracted with EtOAc (30 mL, 3×). The combined organic layer was washed with brine (30 mL), dried over Na₂SO₄, filtered and concentrated under reduced pressure. The resulting residue was purified by preparative TLC (SiO₂, Petroleum ether:EtOAc=0:1) to give the product 8-chloro-N-isobutyl-6-(N-(1- methylcyclopropyl)sulfamoyl)imidazo[1,2-a]pyridine-3-carboxamide (45 mg, 116.92 μmol, 96.39% yield) as a white solid.

¹H NMR (DMSO-d₆, 400 MHz): 9.96 (d, J=1.2 Hz, 1H), 8.78 (t, J=5.2 Hz, 1H), 8.53 (s, 1H), 8.44 (s, 1H), 7.80 (s, 1H), 3.15 (t, J=6.4 Hz, 2H), 1.76-1.93 (m, 1H), 1.14 (s, 3H), 0.93 (d, J=6.4 Hz, 6H), 0.68 (s, 2H), 0.41-0.53 (m, 2H).

### Preparation of Example 78

### N-isobutyl-6-(N-(1-methylcyclopropyl)sulfamoyl)-8-(2-oxa-7-azaspiro[3.5]nonan-7-yl)imidazo[1,2-a]pyridine-3-carboxamide

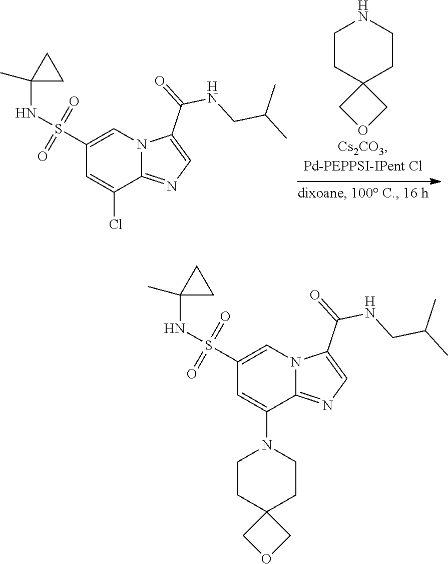

To a solution of 2-oxa-7-azaspiro[3.5]nonane (13.22 mg, 103.93 μmol) in dioxane (0.5 mL) was added 8-chloro-N-isobutyl-6-(N-(1-methylcyclopropyl)sulfamoyl)imidazo[1,2-a]pyridine-3-carboxamide (20 mg, 51.96 μmol), Cs₂CO₃ (33.86 mg, 103.93 μmol) and Pd-PEPPSI-IPentCl o-picoline (2.24 mg, 2.60 μmol) in glove box. The mixture was stirred at 90° C. for 16 h outside of the glove box under Ar. The reaction mixture was cooled to room temperature and concentrated under reduced pressure. The resulting residue was diluted with MeOH (3 mL), filtered and the filtrate was purified by preparative HPLC (column: Unisil 3-100 C18 Ultra 150*50 mm*3 μm; mobile phase: A: 0.225% formic acid in water, B: MeCN; B %: 31%-61%, 7 min) and lyophilized directly to give the product N-isobutyl-6-(N-(1-methylcyclopropyl)sulfamoyl)-8-(2-oxa-7-azaspiro[3.5]nonan-7-yl)imidazo[1,2-a]pyridine-3-carboxamide (0.53 mg, 1.10 μmol, 2.12% yield, 99% purity) as an off-white solid.

RT 0.867 min (method 1); m/z 476.2 (M+H)⁺ (ESI⁺); ¹H NMR (DMSO-d₆, 400 MHz): 9.58 (s, 1H), 8.65 (t, J=5.6 Hz, 1H), 8.36 (s, 1H), 8.25 (s, 1H), 6.85 (s, 1H), 4.39 (s, 4H), 3.47-3.51 (m, 4H), 3.08-3.15 (m, 2H), 1.94-2.00 (m, 4H), 1.79-1.90 (m, 1H), 1.09 (s, 3H), 0.92 (d, J=6.8 Hz, 6H), 0.61-0.71 (m, 2H), 0.37-0.45 (m, 2H).

### Preparation of Example 79

### methyl 6-(N-(1-methylcyclopropyl)sulfamoyl)-8-(2-oxa-7-azaspiro[3.5]nonan-7-yl)imidazo[1,2-a]pyridine-3-carboxylate

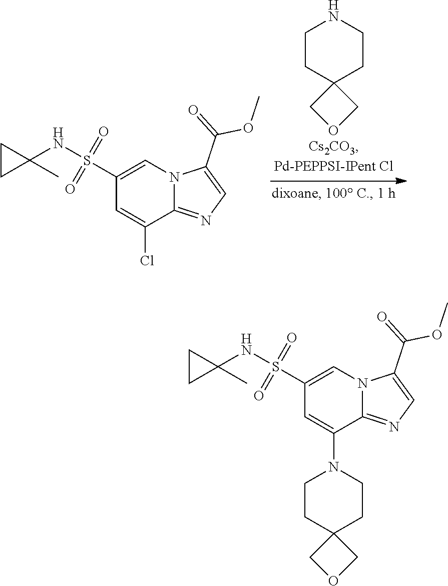 [SMILES: COC(=O)c1cnc2c(Cl)cc(S(=O)(=O)NC3(C)CC3)cn12 ; C1CC2(CCN1)COC2 ; COC(=O)c1cnc2c(N3CCC4(CC3)COC4)cc(S(=O)(=O)NC3(C)CC3)cn12]

A mixture of methyl 8-chloro-6-(N-(1-methylcyclopropyl)sulfamoyl)imidazo[1,2-a]pyridine-3-carboxylate (30 mg, 87.26 μmol), 2-oxa-7-azaspiro[3.5]nonane (31.57 mg, 130.90 μmol, TFA salt), Cs₂CO₃ (85.30 mg, 261.79 μmol), Pd-PEPPSI-IPentCl o-picoline (3.75 mg, 4.36 μmol) in dioxane (0.5 mL) was degassed and purged with N₂ (3×). The reaction mixture was stirred at 100° C. for 12 h under a N₂ atmosphere, then cooled to room temperature, diluted with H₂O (30 mL) and extracted with EtOAc (20 mL, 2×). The combined organic layer was washed with brine (30 mL, 2×), dried over anhydrous Na₂SO₄, filtered and concentrated under vacuum. The resulting residue was purified by preparative-TLC (SiO₂, Petroleum ether/EtOAc=2/1) to give the product methyl 6-(N-(1-methylcyclopropyl)sulfamoyl)-8-(2-oxa-7-azaspiro[3.5]nonan-7-yl)imidazo[1,2-a]pyridine-3-carboxylate (16 mg, 36.82 μmol, 42.20% yield) as a yellow solid.

RT 0.834 min (method 1); m/z 435.2 (M+H)⁺ (ESI⁺); ¹H NMR (CDCl₃, 400 MHz): 9.43 (d, J=1.6 Hz, 1H), 8.20 (s, 1H), 6.84 (d, J=1.6 Hz, 1H), 4.88 (s, 1H), 4.45 (s, 4H), 3.90 (s, 3H), 3.38-3.46 (m, 4H), 2.01-2.13 (m, 4H), 1.24 (s, 3H), 0.73-0.81 (m, 2H), 0.45-0.51 (m, 2H)

### Preparation of Example 80

### Ethyl 8-(4-(dimethylcarbamoyl)piperazin-1-yl)-6-(N-(1-methylcyclopropyl)sulfamoyl)imidazo[1,2-a]pyridine-3-carboxylate

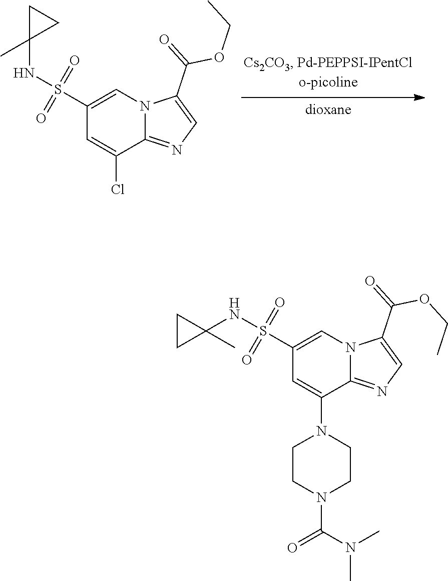 [SMILES: CCOC(=O)c1cnc2c(Cl)cc(S(=O)(=O)NC3(C)CC3)cn12 ; CCOC(=O)c1cnc2c(N3CCN(C(=O)N(C)C)CC3)cc(S(=O)(=O)NC3(C)CC3)cn12]

To a solution of N,N-dimethylpiperazine-1-carboxamide (131.81 mg, 838.43 μmol) in dioxane (1 mL) was added ethyl 8-chloro-6-(N-(1-methylcyclopropyl)sulfamoyl)imidazo[1,2-a]pyridine-3-carboxylate (100 mg, 279.48 μmol), Pd-PEPPSI-IPentCl o-picoline (12.03 mg, 13.97 μmol), Cs₂CO₃ (182.12 mg, 558.95 μmol) were added in a glove box. The mixture was stirred at 100° C. outside of the glove box for 2 h under Ar₂. The mixture was quenched by cold water (50 mL) and was extracted with EtOAc (50 mL, 3×). The combined organic layer was washed with brine (20 mL), dried over Na₂SO₄, filtered and concentrated under reduced pressure. The resulting residue was purified by preparative-TLC (SiO₂, DCM/MeOH=15/1) to give the product ethyl 8-(4-(dimethylcarbamoyl)piperazin-1-yl)-6-(N-(1-methylcyclopropyl)sulfamoyl)imidazo[1,2-a]pyridine-3-carboxylate (100 mg, 188.06 μmol, 67.29% yield, 90% purity) as a brown oil.

RT 0.845 min (Method 1); m/z 479.3 (M+H)⁺ (ESI⁺); ¹H NMR (CDCl₃, 400 MHz): 9.54 (s, 1H), 8.26 (s, 1H), 6.91 (s, 1H), 5.02 (s, 1H), 3.47-3.42 (q, 2H), 3.61-3.60 (m, 4H), 3.53-3.52 (m, 4H), 2.89 (s, 6H), 1.44 (t, 3H), 1.31 (s, 3H), 0.87-0.85 (m, 2H), 0.57-0.54 (m, 2H).

### Preparation of Intermediate 81.1

### 8-(4-(dimethylcarbamoyl)piperazin-1-yl)-6-(N-(1-methylcyclopropyl)sulfamoyl)imidazo[1,2-a]pyridine-3-carboxylic acid

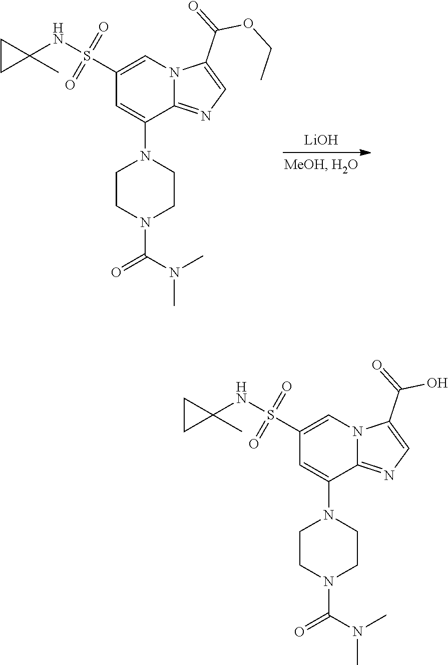 [SMILES: CCOC(=O)c1cnc2c(N3CCN(C(=O)N(C)C)CC3)cc(S(=O)(=O)NC3(C)CC3)cn12 ; "LiOH" ; CN(C)C(=O)N1CCN(c2cc(S(=O)(=O)NC3(C)CC3)cn3c(C(=O)O)cnc23)CC1]

To a solution of ethyl 8-(4-(dimethylcarbamoyl)piperazin-1-yl)-6-(N-(1-methylcyclopropyl)sulfamoyl)imidazo[1,2-a]pyridine-3-carboxylate (10 mg, 18.81 μmol) in MeOH (0.1 mL), H₂O (0.02 mL) was added LiOH—H₂O (2.37 mg, 56.42 μmol). The mixture was stirred at 20° C. for 3 h. Then, the pH of the reaction was adjusted to 1-2 with 1M hydrochloric acid aqueous solution and the solution was extracted with EtOAc (10 mL, 3×). The combined organic layer was washed with brine (20 mL), dried over Na₂SO₄, filtered and concentrated under reduced pressure. The resulting residue was purified by preparative HPLC (column: Phenomenex C18 75*30 mm*3 μm; mobile phase: A: 0.225% formic acid in water, B: MeCN; B %: 20%-50%, 7 min) and lyophilized directly to give the product 8-(4-(dimethylcarbamoyl)piperazin-1-yl)-6-(N-(1-methylcyclopropyl)sulfamoyl)imidazo[1,2-a]pyridine-3-carboxylic acid (3.45 mg, 7.66 μmol, 40.72% yield, 100% purity) as a yellow solid.

RT 0.794 min (method 1); m/z 451.0 (M+H)⁺ (ESI⁺); ¹H NMR (DMSO-d₆, 400 MHz): 9.64 (s, 1H), 8.24-8.20 (m, 1H), 8.23 (s, 1H), 8.04 (s, 1H), 6.84 (s, 1H), 3.56-3.55 (m, 4H), 3.30-3.33 (m, 4H), 2.79 (s, 6H), 1.10 (s, 3H), 0.69-0.66 (m, 2H), 0.43-0.40 (m, 2H).

### Preparation of Example 81

### Methyl 8-(4-(dimethylcarbamoyl)piperazin-1-yl)-6-(N-(1-methylcyclopropyl)sulfamoyl)imidazo[1,2-a]pyridine-3-carboxylate

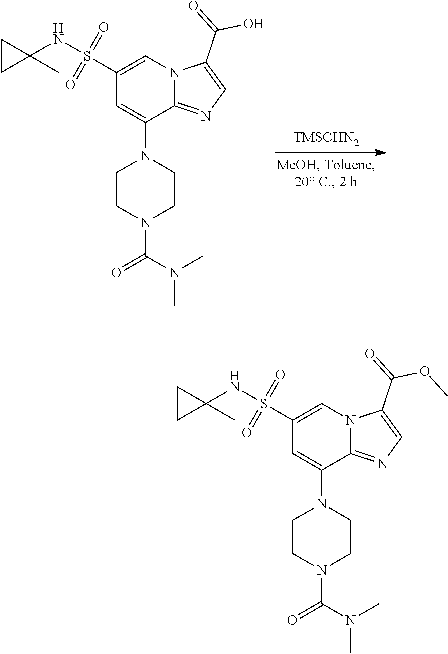 [SMILES: CN(C)C(=O)N1CCN(c2cc(S(=O)(=O)NC3(C)CC3)cn3c(C(=O)O)cnc23)CC1 ; COC(=O)c1cnc2c(N3CCN(C(=O)N(C)C)CC3)cc(S(=O)(=O)NC3(C)CC3)cn12]

To a solution of 8-(4-(dimethylcarbamoyl)piperazin-1-yl)-6-(N-(1-methylcyclopropyl)sulfamoyl)imidazo[1,2-a]pyridine-3-carboxylic acid (10 mg, 22.20 μmol) in MeOH (0.15 mL) and toluene (0.5 mL) was added TMSCHN2 (2 M, 22.20 μL). The mixture was stirred at 20° C. for 2 h and concentrated under reduced pressure. The resulting residue was purified by preparative HPLC (column: Phenomenex C18 75*30 mm*3 μm; mobile phase: A: 0.225% formic acid in water, B: MeCN; B %: 28%-58%, 7 min) and lyophilized directly to give the product methyl 8-(4-(dimethylcarbamoyl)piperazin-1-yl)-6-(N-(1-methylcyclopropyl)sulfamoyl)imidazo[1,2-a]pyridine-3-carboxylate (5.36 mg, 11.54 μmol, 51.98% yield, 100% purity) as a yellow gum.

RT 0.856 min (method 1); m/z 465.0 (M+H)⁺ (ESI⁺); ¹H NMR (CDCl₃, 400 MHz): 9.52 (s, 1H), 8.26 (s, 1H), 6.93 (s, 1H), 5.17 (s, 1H), 3.97 (s, 3H), 3.61-3.59 (m, 4H), 3.52-3.51 (m, 4H), 2.88 (s, 6H), 1.31 (s, 3H), 0.86-0.83 (m, 2H), 0.56-0.53 (m, 2H).

### Preparation of Example 82

### 8-(4-(dimethylcarbamoyl)piperazin-1-yl)-6-(N-(1-methylcyclopropyl)sulfamoyl)-N-(oxetan-3-yl)imidazo[1,2-a]pyridine-3-carboxamide

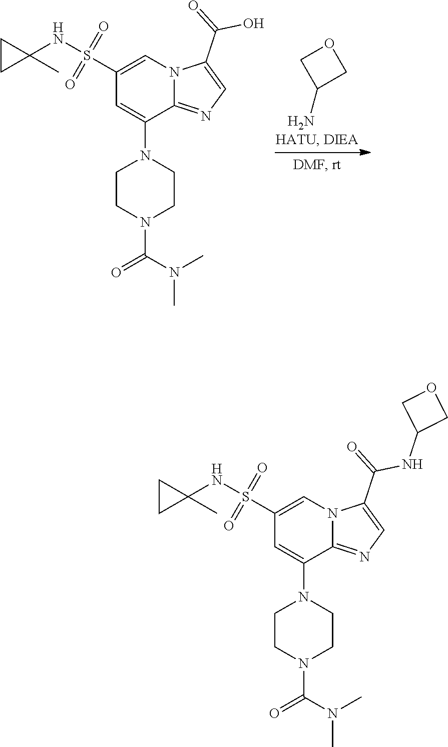 [SMILES: CN(C)C(=O)N1CCN(c2cc(S(=O)(=O)NC3(C)CC3)cn3c(C(=O)O)cnc23)CC1 ; NC1COC1 ; CN(C)C(=O)N1CCN(c2cc(S(=O)(=O)NC3(C)CC3)cn3c(C(=O)NC4COC4)cnc23)CC1]

To a mixture of 8-(4-(dimethylcarbamoyl)piperazin-1-yl)-6-(N-(1-methylcyclopropyl)sulfamoyl)imidazo[1,2-a]pyridine-3-carboxylic acid (20 mg, 44.39 μmol), oxetan-3-amine (3.89 mg, 53.27 μmol) in DMF (0.5 mL) was added DIEA (8.61 mg, 66.59 μmol, 11.60 μL), and HATU (25.32 mg, 66.59 μmol). The mixture was stirred at 20° C. for 1 h. The resulting mixture was diluted with water (10 mL), extracted with EtOAc (10 mL, 3×). The combined organic layer was washed with brine (20 mL), dried over Na₂SO₄, filtered and concentrated under reduced pressure. The resulting mixture was purified by preparative HPLC (column: Phenomenex Synergi C18 150*25 mm*10 μm; mobile phase: A: 0.225% formic acid in water, B: MeCN; B %: 20%-50%, 10 min) and lyophilized directly to give the product 8-(4-(dimethylcarbamoyl)piperazin-1-yl)-6-(N-(1-methylcyclopropyl)sulfamoyl)-N-(oxetan-3-yl)imidazo[1,2-a]pyridine-3-carboxamide (5.89 mg, 11.32 μmol, 25.50% yield, 97.180% purity) as a light yellow solid.

RT 0.770 min (method 1); m/z 506.2 (M+H)⁺ (ESI⁺); ¹H NMR (DMSO-d₆, 400 MHz): 9.56 (d, J=1.6 Hz, 1H), 9.29 (d, J=6.8 Hz, 1H), 8.43 (s, 1H), 8.27 (s, 1H), 6.89 (d, J=1.6 Hz, 1H), 5.01-5.12 (m, 1H), 4.81 (t, J=6.8 Hz, 2H), 4.62 (t, J=6.48 Hz, 2H), 3.53-3.60 (m, 4H), 3.30-3.34 (m, 4H), 2.79 (s, 6H), 1.09 (s, 3H), 0.61-0.71 (m, 2H), 0.36-0.46 (in, 2H)

Compounds listed in the table below were prepared according to the corresponding general procedures or, when stated in a similar way to the related example, and starting from the corresponding intermediates or examples.

| Cpd | | | | | |
|---|---|---|---|---|---|
| number | Structure | Yield (%) | Procedure | LC/MS | 1H NMR |
| 83 | 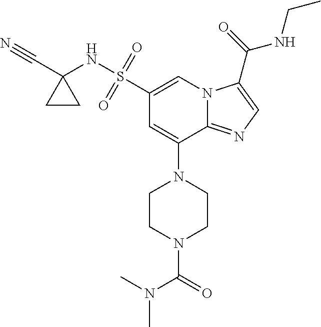 [SMILES: CCNC(=O)c1cnc2c(N3CCN(C(=O)N(C)C)CC3)cc(S(=O)(=O)NC3(C#N)CC3)cn12] | 18.9% (yield after 2 steps) | Procedure 4 followed by Procedure 1 | RT 0.624 min (Method 1); m/z 489.1 (M + H)⁺ (ESI⁺) | ¹H NMR (DMSO-d₆, 400 MHz) 9.72 (s, 1H), 9.45- 9.10(m, 1H), 8.75-8.62 (m, 1H), 8.35 (s, 1H), 6.88 (s, 1H), 3.67-3.55 (m, 4H), 3.50-3.38 (m, 6H), 2.80 (s, 6H), 1.46-1.39 (m, 2H), 1.35-1.28 (m, 2H), 1.18 (t, J = 7.2 Hz, 3H). |
| 84 | 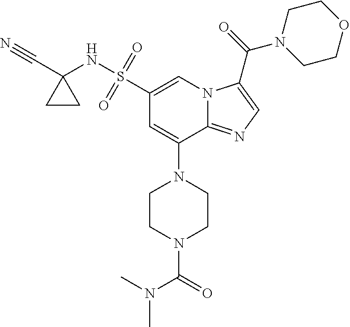 [SMILES: CN(C)C(=O)N1CCN(c2cc(S(=O)(=O)NC3(C#N)CC3)cn3c(C(=O)N4CCOCC4)cnc23)CC1] | 1.28% (yield after 2 steps) | Procedure 4 followed by Procedure 1 | RT 0.600 min (method 1); m/z 531.1 (M + H)⁺ (ESI⁺) | ¹H NMR (DMSO-d₆, 400 MHz): 9.00 (s, 1H), 8.06 (s, 1H), 6.86 (s, 1H), 3.97- 3.45 (m, 16H), 2.80 (s, 6H), 1.38-1.44 (m, 2H), 1.28-1.34 (m, 2H) |
| 85 | 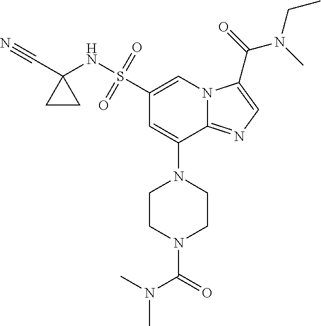 [SMILES: CCN(C)C(=O)c1cnc2c(N3CCN(C(=O)N(C)C)CC3)cc(S(=O)(=O)NC3(C#N)CC3)cn12] | 2.80% (yield after 2 steps) | Procedure 4 followed by Procedure 1 | RT 0.622 min (Method 1); m/z 503.1 (M + H)⁺ (ESI⁺) | ¹H NMR (DMSO-d₆, 400 MHz) 9.32 (s, 1H), 9.16 (s, 1H), 7.96-8.19 (m, 1H), 6.86 (d, J = 1.2 Hz, 1H), 3.55-3.65 (m, 6H), 3.34-3.37 (m, 4H), 3.08- 3.25 (m, 3H), 2.80 (s, 6H), 1.42-1.51 (m, 2H), 1.32- 1.39 (m, 2H), 1.19-1.25 (m, 3H). |
| 86 | 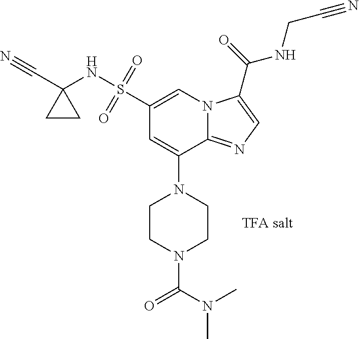 [SMILES: CN(C)C(=O)N1CCN(c2cc(S(=O)(=O)NC3(C#N)CC3)cn3c(C(=O)NCC#N)cnc23)CC1] | 2.44% (yield after 2 steps) | Procedure 4 followed by Procedure 1 | RT 0.789 min (Method 1); m/z 500.2 (M + H)⁺ (ESI⁺) | ¹H NMR (DMSO-d₆, 400 MHz) 9.65 (d, J = 1.6 Hz, 1H), 9.39-9.47 (m, 1H), 9.36 (s, 1H), 8.42 (s, 1H), 6.93 (s, 1H), 4.41 (d, J = 5.2 Hz, 2H), 3.61 (d, J = 2.0 Hz, 4H), 3.28 (d, J = 1.0 Hz, 4H), 2.80 (s, 6H), 1.40-1.53 (m, 2H), 1.26- 1.39 (m, 2H) |
| 87 | 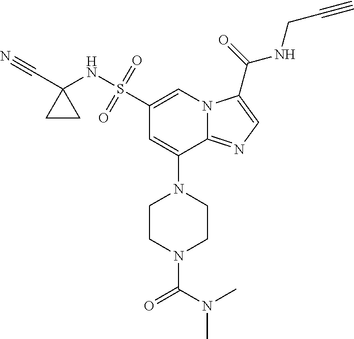 [SMILES: C#CCNC(=O)c1cnc2c(N3CCN(C(=O)N(C)C)CC3)cc(S(=O)(=O)NC3(C#N)CC3)cn12] | 5.76% (yield after 3 steps) | Procedure 4 then Procedure 1 followed by Procedure 11 | RT 0.635 min (method 1); m/z 499.0 (M + H)⁺ (ESI⁺) | ¹H NMR (DMSO-d₆, 400 MHz): 9.64 (s, 1H), 9.15 (t, J = 5.6 Hz, 1H), 8.40 (s, 1H), 8.36 (s, 1H), 6.90 (s, 1H), 4.13-4.11 (m, 2H), 3.58 (s, 4H), 3.34 (s, 4H), 3.20 (t, J = 2.4 Hz, 1H), 2.80 (s, 6H), 1.31-1.40 (m, 2H), 1.23-1.31 (m, 2H). |
| 88 | 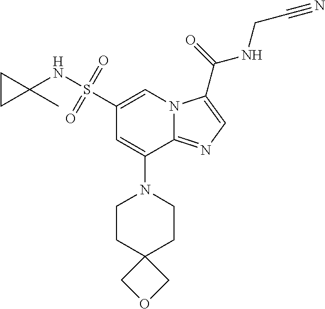 | 17.14% (yield after 3 steps) | Procedure 1 then Procedure 9 followed by Procedure 5 | RT 0.473 min (method 1); m/z 495.3 (M + H)⁺ (ESI⁺) | ¹H NMR (DMSO-d₆, 400 MHz): 9.52 (d, J = 1.6 Hz, 1H), 9.34-9.41 (m, 1H), 8.37 (s, 1H), 8.28 (s, 1H), 6.89 (d, J = 1.2 Hz, 1H), 4.34-4.44 (m, 6H), 3.45-3.54 (m, 4H), 1.93- 2.02 (m, 4H), 1.09 (s, 3H), 0.63-0.70 (m, 2H), 0.36-0.45 (m, 2H) |
| 89 | 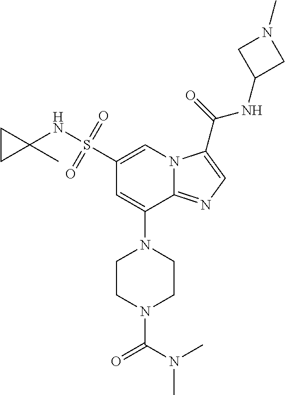 [SMILES: CN1CC(NC(=O)c2cnc3c(N4CCN(C(=O)N(C)C)CC4)cc(S(=O)(=O)NC4(C)CC4)cn23)C1] | 17.75% (yield after 3 steps) | Procedure 1 then Procedure 9 followed by Procedure 5 | RT 0.701 min (method 1); m/z 519.3 (M + H)⁺ (ESI⁺) | ¹H NMR (CDCl₃, 400 MHz) 9.68 (d, J = 1.6 Hz, 1H), 9.35-9.63 (m, 1H), 8.56 (s, 1H), 8.29-8.46 (m, 1H), 6.88 (d, J = 1.6 Hz, 1H), 4.90-5.02 (m, 1H), 4.12-4.20 (m, 2H), 3.92-4.03 (m, 2H), 3.56- 3.62 (m, 4H), 3.48-3.55 (m, 4H), 2.89 (s, 6H), 2.74 (s, 3H), 1.31 (s, 3H), 0.82-0.87 (m, 2H), 0.50-0.56 (m, 2H) |
| 90 | 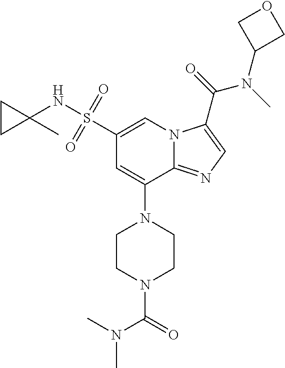 [SMILES: CN(C)C(=O)N1CCN(c2cc(S(=O)(=O)NC3(C)CC3)cn3c(C(=O)N(C)C4COC4)cnc23)CC1] | 10.84% (yield after 3 steps) | Procedure 1 then Procedure 9 followed by Procedure 5 | RT 0.807 min (method 1); m/z 520.1 (M + H)+ (ESI+). | ¹H NMR (CDCl₃, 400 MHz): 9.22 (s, 1H), 7.84 (s, 1H), 6.87 (d, J = 1.6 Hz, 1H), 5.53-5.48 (m, 1H), 5.02 (s, 1H), 4.98- 4.92 (m, 2H), 4.89-4.84 (m, 2H), 3.61-3.59(m, 4H), 3.53-3.51 (m, 4H), 3.42 (s, 3H), 2.90 (s, 6H), 1.32 (s, 3H), 0.88-0.85 (m, 2H), 0.58-0.55 (m, 2H). |
| 91 | 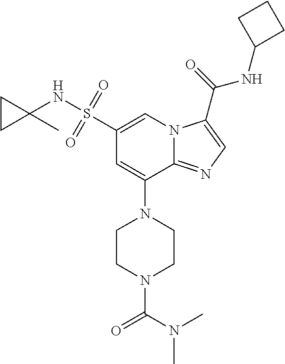 [SMILES: CN(C)C(=O)N1CCN(c2cc(S(=O)(=O)NC3(C)CC3)cn3c(C(=O)NC4CCC4)cnc23)CC1] | 69.95% (yield after 3 steps) | Procedure 1 then Procedure 9 followed by Procedure 5 | RT 0.567 min (method 3); m/z 504.1 (M + H)+ (ESI+). | ¹H NMR (DMSO-d₆, 400 MHz): 9.60 (d, J = 1.6 Hz, 1H), 8.78 (d, J = 7.6 Hz, 1H), 8.37 (s, 1H), 8.25 (s, 1H), 6.87 (d, J = 1.6 Hz, 1H), 4.50-4.40 (m,1H), 3.57-3.55 (m, 4H), 3.32- 3.30 (m, 4H), 2.79 (s, 6H), 2.31-2.21 (m, 2H), 2.13- 2.07 (m, 2H), 1.75-1.68 (m, 2H), 1.09 (s, 3H), 0.68-0.65 (m, 2H), 0.43- 0.40 (m, 2H). |
| 92 | 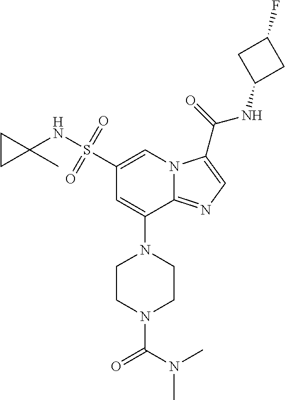 [SMILES: CN(C)C(=O)N1CCN(c2cc(S(=O)(=O)NC3(C)CC3)cn3c(C(=O)N[C@H]4C[C@@H](F)C4)cnc23)CC1] | 41.72% (yield after 3 steps) | Procedure 1 then Procedure 9 followed by Procedure 5 | RT 0.846 min (method 1); m/z 522.1 (M + H)+ (ESI+). | ¹H NMR (DMSO-d₆, 400 MHz): 9.59 (d, J = 1.6 Hz, 1H), 8.85 (d, J = 7.2 Hz, 1H), This is the maide proton 8.37 (s, 1H), 8.26 (s, 1H), 6.88 (d, J = 1.6 Hz, 1H), 5.01-4.80 (m, 1H), 4.09-3.99 (m, 1H), 3.57-3.55 (m, 4H), 3.34- 3.32 (m, 4H), 2.80 (s, 6H), 2.54-2.50 (m, 4H), 1.09 (s, 3H), 0.68-0.65 (m, 2H), 0.43-0.40 (m, 2H) |
| 93 | 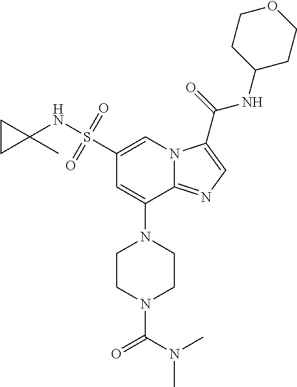 [SMILES: CN(C)C(=O)N1CCN(c2cc(S(=O)(=O)NC3(C)CC3)cn3c(C(=O)NC4CCOCC4)cnc23)CC1] | 21.11% (yield after 3 steps) | Procedure 1 then Procedure 9 followed by Procedure 5 | RT 0.533 min (method 3); m/z 534.2 (M + H)+ (ESI+) | ¹H NMR (400 MHz, DMSO-d₆): 9.62 (d, J = 1.6 Hz, 1H), 8.51 (d, J = 7.6 Hz, 1H), 8.38 (s, 1H), 8.26 (s, 1H), 6.88 (s, 1H), 4.10-4.01 (m, 1H), 3.95- 3.86 (m, 2H), 3.56-3.55 (m, 4H), 3.44-3.42 (m, 2H), 3.34-3.33 (m, 4H), 2.79 (s, 6H), 1.87-1.76 (m, 2H), 1.64-1.54 (m, 2H), 1.10 (s, 3H), 0.69- 0.64 (m, 2H), 0.44-0.39 (m, 2H) |
| 94 | 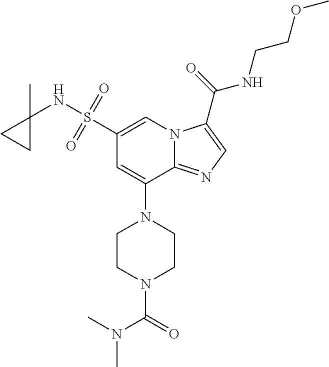 [SMILES: COCCNC(=O)c1cnc2c(N3CCN(C(=O)N(C)C)CC3)cc(S(=O)(=O)NC3(C)CC3)cn12] | 39.4% (yield after 3 steps) | Procedure 1 then Procedure 9 followed by Procedure 5 | RT 0.537 min (method 3); m/z 508.4 (M + H)+ (ESI+) | ¹H NMR (400 MHz, DMSO-d₆): 9.63 (d, J = 1.6 Hz, 1H), 8.80-8.67 (m, 1H), 8.39 (s, 1H), 8.26 (s, 1H), 6.89 (d, J = 1.6 Hz, 1H), 3.58-3.56 (m, 4H), 3.51-3.45 (m, 4H), 3.35- 3.33 (m, 2H),3.31 (s, 2H), 3.30 (s, 3H), 2.80 (s, 6H), 1.11 (s, 3H), 0.69-0.66 (m, 2H), 0.44-0.41 (m, 2H). |
| 95 | 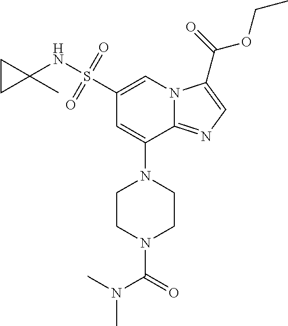 [SMILES: CCOC(=O)c1cnc2c(N3CCN(C(=O)N(C)C)CC3)cc(S(=O)(=O)NC3(C)CC3)cn12] | 44.85% | Procedure 1 | RT 0.919 min (method 1); m/z 479.2 (M + H)+ (ESI+) | ¹H NMR (CDCl₃, 400 MHz): 9.54 (s, 1H), 8.26 (s, 1H), 6.91 (s, 1H), 5.02 (s, 1H), 4.48-4.42 (m, 2H), 3.58-3.56 (m, 4H), 3.51-3.45 (m, 4H), 2.89 (s, 6H), 1.46 (t, J = 7.2 Hz 3H), 1.31 (s, 3H), 0.88- 0.83 (m, 2H), 0.57-0.54 (m, 2H). |

### Preparation of Intermediate 96.1

### 6-amino-5-bromo-pyridine-3-sulfonyl chloride

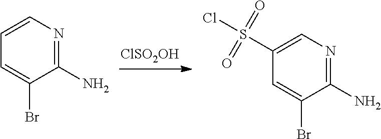

2 batches were conducted in parallel and combined for the work-up.

A solution of 3-bromopyridin-2-amine (10 g, 57.80 mmol) in sulfurochloridic acid (67.35 g, 578.00 mmol, 38.49 mL) was stirred at 140° C. for 1 hr. The reaction mixture was cooled to 0° C. and poured into ice-water (500 mL). The resulting mixture (two batches were combined together) was filtered. The solid was collected, dried under reduced pressure and triturated by petroleum ether:EtOAc (1:1, 100 mL) at 20° C. for 2 h. Then, the mixture was filtered and the solid was collected followed by drying under reduced pressure. The crude compound was triturated by HCl/dioxane (4N, 110 mL) at 20° C. for 1 h to give after filtration the product 6-amino-5-bromo-pyridine-3-sulfonyl chloride (26.5 g, 97.11 mmol, 72.91% yield, 99.5% purity) as white solid.

RT 0.817 min (method 1); m/z 272.9 (M+H)⁺ (ESI⁺); ¹H NMR (DMSO-d₆, 400 MHz): 8.25 (d, J=1.6 Hz. 1H), 8.12 (d, J=1.6 Hz. 1H).

### Preparation of Intermediate 96.2

### 6-amino-5-bromo-N-(1-methylcyclopropyl)pyridine-3-sulfonamide

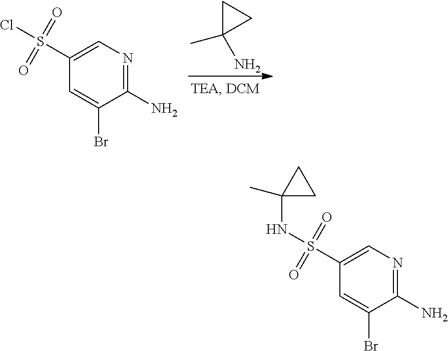

2 batches were conducted in parallel and combined for the work-up.

At 0° C., to a mixture of 6-amino-5-bromo-pyridine-3-sulfonyl chloride (12.9 g, 47.51 mmol) in DCM (130 mL) was added TEA (14.42 g, 142.53 mmol, 19.84 mL), followed by 1-methylcyclopropan-1-amine (5.62 g, 52.26 mmol, HCl salt) portionwise. The reaction mixture was stirred at 15° C. for 1 hr and concentrated under reduced pressure. The residue (coming from 2 batches) was purified by column chromatography (SiO₂, Petroleum ether/Ethyl acetate=5/1 to 4/5) to give the product 6-amino-5-bromo-N-(1-methylcyclopropyl)pyridine-3-sulfonamide (22 g, 68.19 mmol, 71.76% yield, 94.9% purity) as white solid.

RT 0.735 min (method 1); m/z 305.9 (M+H)⁺ (ESI⁺); ¹H NMR (DMSO-d₆, 400 MHz): 8.27 (d, J=2.0 Hz. 1H), 7.92 (d, J=2.0 Hz. 1H), 7.85 (s, 1H), 7.16 (br, 2H), 1.09 (s, 3H), 0.62 (t, J=4.8 Hz. 2H), 0.40 (t, J=5.2 Hz, 2H).

### Preparation of Intermediate 96.3

### 8-bromo-N-(1-methylcyclopropyl)imidazo[1,2-a]pyridine-6-sulfonamide

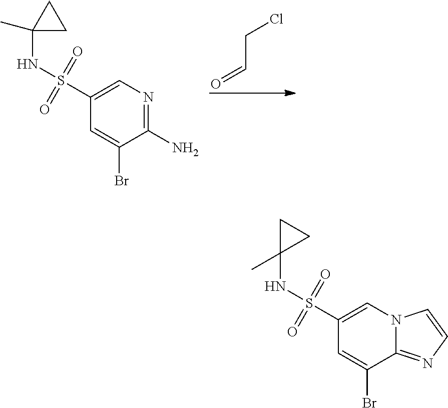

The mixture of 6-amino-5-bromo-N-(1-methylcyclopropyl)pyridine-3-sulfonamide (12 g, 39.19 mmol) in 2-chloroacetaldehyde/H₂O (260.27 g, 1.33 mol, 213.34 mL, 40% purity) was stirred at 100° C. for 1 h. The resulting mixture was cooled to room temperature and diluted by H₂O (300 mL). The mixture was extracted with EtOAc (200 mL, 3×). The combined organic layer was washed with brine (400 mL), dried over anhydrous Na₂SO₄, filtered and dried under reduced pressure. The residue was purified by column chromatography (SiO₂, Petroleum ether/Ethyl acetate=5/1 to 4/5) to give the product 8-bromo-N-(1-methylcyclopropyl)imidazo[1,2-a]pyridine-6-sulfonamide (12.9 g, 39.07 mmol, 99.68% yield, 100% purity) as white solid.

RT 0.628 min (method 1); m/z 329.9 (M+H)⁺ (ESI⁺); ¹H NMR (DMSO-d₆, 400 MHz): 9.25 (d, J=1.6 Hz, 1H), 8.32 (s, 1H), 8.25 (d, J=1.2 Hz, 1H), 7.77 (d, J=1.2 Hz, 1H), 7.73 (d, J=1.6 Hz, 1H), 1.13 (s, 3H), 0.67 (t, J=4.8 Hz. 2H), 0.44 (t, J=5.2 Hz, 2H).

### Preparation of Intermediate 96.4

### N,N-dimethyl-4-(6-(N-(1-methylcyclopropyl)sulfamoyl)imidazo[1,2-a]pyridin-8-yl)piperazine-1-carboxamide

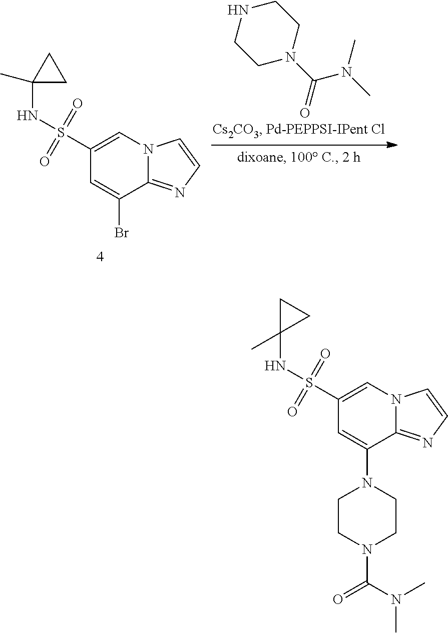 [SMILES: CC1(NS(=O)(=O)c2cc(Br)c3nccn3c2)CC1 ; CN(C)C(=O)N1CCNCC1 ; CN(C)C(=O)N1CCN(c2cc(S(=O)(=O)NC3(C)CC3)cn3ccnc23)CC1]

To a solution of N,N-dimethylpiperazine-1-carboxamide (476.11 mg, 3.03 mmol) in dioxane (5 mL) were added 8-bromo-N-(1-methylcyclopropyl)imidazo[1,2-a]pyridine-6-sulfonamide (0.5 g, 1.51 mmol), Cs₂CO₃ (986.73 mg, 3.03 mmol) and Pd-PEPPSI-IPentCl (65.15 mg, 75.71 μmol) in glove box. The mixture was then stirred at 100° C. for 2 h outside of the glove box under Ar. The reaction mixture was cooled to room temperature, quenched by H₂O (50 mL) and extracted with EtOAc (50 mL; 3×). The combined organic layers were washed with brine (50 mL), dried over anhydrous Na₂SO₄, filtered, concentrated under reduced pressure. The residue was purified by flash silica gel chromatography (ISCO®; 12 g SepaFlash® Silica Flash Column, Eluent of 60-100% Ethylacetate/Petroleum@75 mL/min) to give the product N,N-dimethyl-4-(6-(N-(1-methylcyclopropyl)sulfamoyl)imidazo[1,2-a]pyridin-8-yl)piperazine-1-carboxamide (0.3 g, 738.00 μmol, 48.74% yield) as a brown solid.

RT 0.716 min (method 1); m/z 407.1 (M+H)⁺ (ESI⁺); ¹H NMR (DMSO-d₆, 400 MHz): 8.76 (s, 1H), 8.16 (s, 1H), 8.10 (d, J=1.2 Hz, 1H), 7.61 (d, J=1.2 Hz, 1H), 6.66 (s, 1H), 3.60-3.56 (m, 4H), 3.32-3.28 (m, 4H), 2.80 (s, 6H), 1.11 (s, 3H), 0.71-0.65 (m, 2H), 0.37-0.44 (m, 2H).

### Preparation of Intermediate 96.5

### 4-(3-iodo-6-(N-(1-methylcyclopropyl)sulfamoyl)imidazo[1,2-a]pyridin-8-yl)-N,N-dimethylpiperazine-1-carboxamide

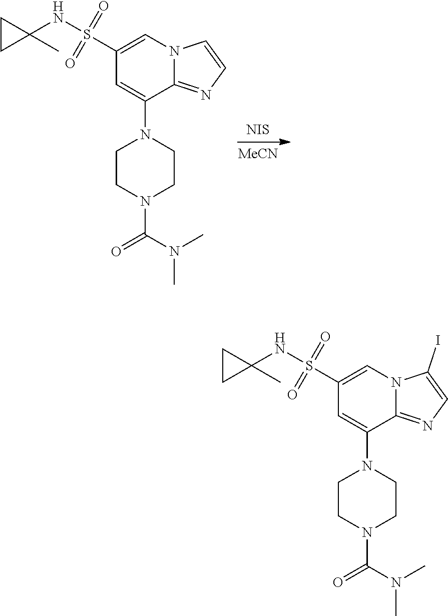

To a solution of N,N-dimethyl-4-(6-(N-(1-methylcyclopropyl)sulfamoyl)imidazo[1,2-a]pyridin-8-yl)piperazine-1-carboxamide (2.75 g, 6.77 mmol) in MeCN (30 mL) was added a solution of NIS (1.67 g, 7.44 mmol) in MeCN (20 mL) at 0° C. The reaction mixture was stirred at 15° C. for 0.5 h and concentrated under reduced pressure. The resulting residue was purified by prep-HPLC (column: Phenomenex luna C18 150*40 mm*15 μm; mobile phase: A: 0.225% formic acid in water; B: MeCN; B %: 37%-67%, 10 min) to give an impure product which was further purified by flash silica gel column (ISCO®; 24 g SepaFlash® Silica Flash Column, Eluent of 50-100% Ethyl acetate/Petroleum ether gradient @45 mL/min) to give the product 4-(3-iodo-6-(N-(1-methylcyclopropyl)sulfamoyl)imidazo[1,2-a]pyridin-8-yl)-N,N-dimethylpiperazine-1-carboxamide (2.1 g, 3.94 mmol, 58.31% yield, 100% purity) as an off-white solid.

RT 0.673 min (method 1); m/z 533.1 (M+H)⁺ (ESI⁺); ¹H NMR (CDCl₃, 400 MHz): 8.44 (d, J=1.2 Hz, 1H), 7.72 (s, 1H), 6.72 (s, 1H), 5.04 (s, 1H), 3.70-3.55 (m, 4H), 3.54-3.45 (m, 4H), 2.89 (s, 6H), 1.32 (s, 3H), 0.85 (t, J=6.4 Hz, 2H), 0.56 (t, J=6.4 Hz, 2H)

### Preparation of Intermediate 96.6

### 4-(3-bromo-6-(N-(1-methylcyclopropyl)sulfamoyl)imidazo[1,2-a]pyridin-8-yl)-N,N-dimethylpiperazine-1-carboxamide

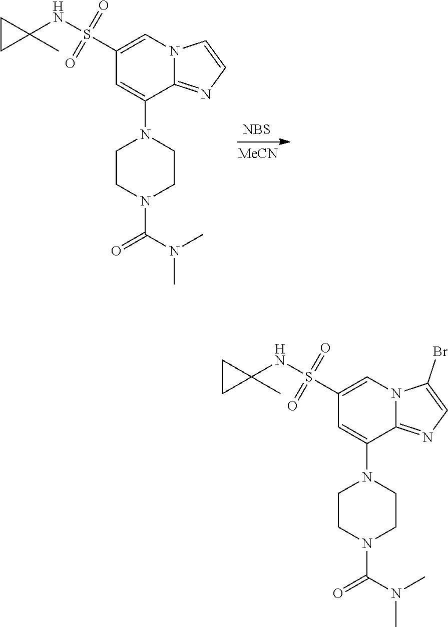

Intermediate 96.2 was prepared according to general procedure 3 and obtained with a yield of 29.86%

RT 0.759 min (method 1); m/z 487.1 (M+H)⁺ (ESI⁺); ¹H NMR (DMSO-d₆, 400 MHz): 8.33 (s, 1H), 8.21 (s, 1H), 7.82 (s, 1H), 6.79 (s, 1H), 3.62 (s, 4H), 3.33 (s, 4H), 2.80 (s, 6H), 1.12 (s, 3H), 0.61-0.72 (m, 2H), 0.39-0.47 (m, 2H).

### Preparation of Intermediate 96.7

### tert-butyl ((3-bromo-8-(4-(dimethylcarbamoyl)piperazin-1-yl)imidazo[1,2-a]pyridin-6-yl)sulfonyl)(1-methylcyclopropyl)carbamate

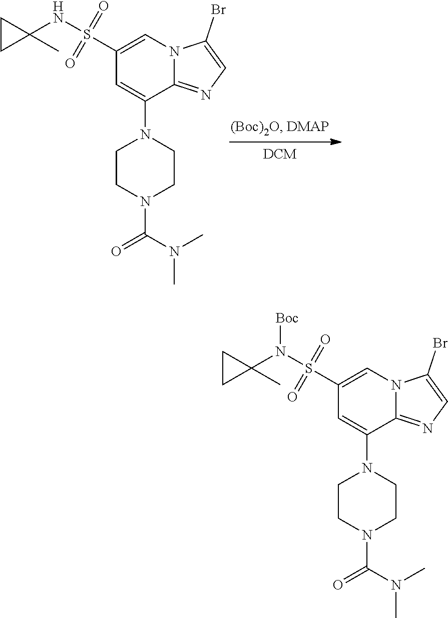 [SMILES: CN(C)C(=O)N1CCN(c2cc(S(=O)(=O)NC3(C)CC3)cn3c(Br)cnc23)CC1 ; CN(C)C(=O)N1CCN(c2cc(S(=O)(=O)N(C(=O)OC(C)(C)C)C3(C)CC3)cn3c(Br)cnc23)CC1]

To a mixture of 4-(3-bromo-6-(N-(1-methylcyclopropyl)sulfamoyl)imidazo[1,2-a]pyridin-8-yl)-N,N-dimethylpiperazine-1-carboxamide (100 mg, 206.02 μmol) in DCM (1.5 mL) was added Boc₂O (67.44 mg, 309.03 μmol, 70.99 μL) and DMAP (5.03 mg, 41.20 μmol) in order. The reaction mixture was stirred at 20° C. for 2 h and concentrated under reduced pressure. The resulting residue was purified by preparative-TLC (SiO₂, PE:EA=0:1) to give the product tert-butyl ((3-bromo-8-(4-(dimethylcarbamoyl)piperazin-1-yl)imidazo[1,2-a]pyridin-6-yl)sulfonyl)(1-methylcyclopropyl)carbamate (71 mg, 119.43 μmol, 57.97% yield, 98.49% purity) as colorless oil.

RT 0.811 min (method 1); m/z 585.1 (M+H)⁺ (ESI⁺); ¹H NMR (CDCl₃, 400 MHz): 8.51 (s, 1H), 7.64 (s, 1H), 6.79 (s, 1H), 3.70-3.60 (m, 4H), 3.60-3.50 (m, 4H), 2.89 (s, 6H), 1.59 (s, 3H), 1.41 (s, 9H), 1.12-0.94 (m, 4H).

### Preparation of Intermediate 96.8

### tert-butyl ((8-(4-(dimethylcarbamoyl)piperazin-1-yl)-3-(5-methylpyridin-2-yl)imidazo[1,2-a]pyridin-6-yl)sulfonyl)(1-methylcyclopropyl)carbamate

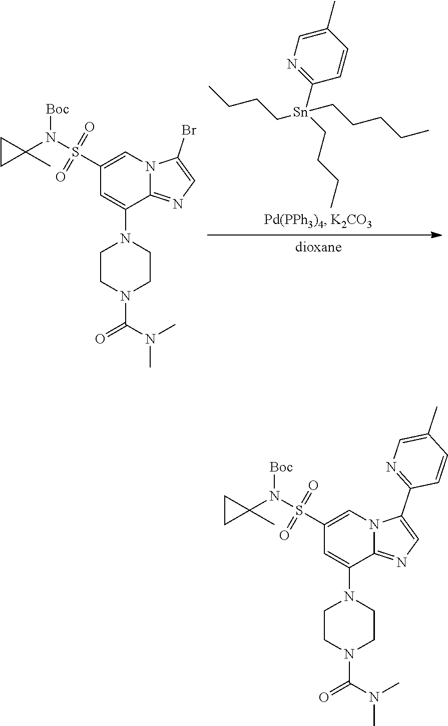 [SMILES: CN(C)C(=O)N1CCN(c2cc(S(=O)(=O)N(C(=O)OC(C)(C)C)C3(C)CC3)cn3c(Br)cnc23)CC1 ; CCCC[CH2][Sn]([CH2]CCC)([CH2]CCC)[c]1ccc(C)cn1 ; Cc1ccc(-c2cnc3c(N4CCN(C(=O)N(C)C)CC4)cc(S(=O)(=O)N(C(=O)OC(C)(C)C)C4(C)CC4)cn23)nc1]

To a solution of tert-butyl ((3-bromo-8-(4-(dimethylcarbamoyl)piperazin-1-yl)imidazo[1,2-a]pyridin-6-yl)sulfonyl)(1-methylcyclopropyl)carbamate (12 mg, 20.49 μmol) in dioxane (1 mL) were added 2-(dibutyl(pentyl)stannyl)-5-methylpyridine (23.50 mg, 61.48 μmol), K₂CO₃ (5.67 mg, 40.99 μmol) and Pd(PPh₃)₄ (2.37 mg, 2.05 μmol) successively. The reaction mixture was degassed, purged with N₂ (3×) and stirred at 80° C. for 16 h. The reaction mixture was filtered and filtrate was concentrated under reduced pressure. The resulting residue was purified by preparative-HPLC (column: Phenomenex luna C18 150*25 mm*10 μm; mobile phase: A: 0.225% formic acid in water, B: MeCN; B %: 58%-88%, 10 min) to give the product tert-butyl ((8-(4-(dimethylcarbamoyl)piperazin-1-yl)-3-(5-methylpyridin-2-yl)imidazo[1,2-a]pyridin-6-yl)sulfonyl)(1-methylcyclopropyl)carbamate (12 mg, 20.08 μmol, 97.96% yield, 100% purity) as an off-white solid.

RT 1.003 min (method 1); m/z 598.3 (M+H)⁺ (ESI⁺); ¹H NMR (CDCl₃, 400 MHz): 10.28 (d, J=1.2 Hz, 1H), 8.51 (s, 1H), 8.07 (s, 1H), 7.65 (d, J=8 Hz, 1H), 7.58 (dd, J=2.0 Hz, J=8 Hz, 1H), 6.93 (d, J=1.2 Hz, 1H), 3.65-3.50 (m, 8H), 2.90 (s, 6H), 2.40 (s, 3H), 1.64 (s, 3H), 1.34 (s, 9H), 1.25-1.24 (m, 2H), 1.10-0.95 (m, 2H).

### Preparation of Example 96

### N,N-dimethyl-4-(6-(N-(1-methylcyclopropyl)sulfamoyl)-3-(5-methylpyridin-2-yl)imidazo[1,2-a]pyridin-8-yl)piperazine-1-carboxamide

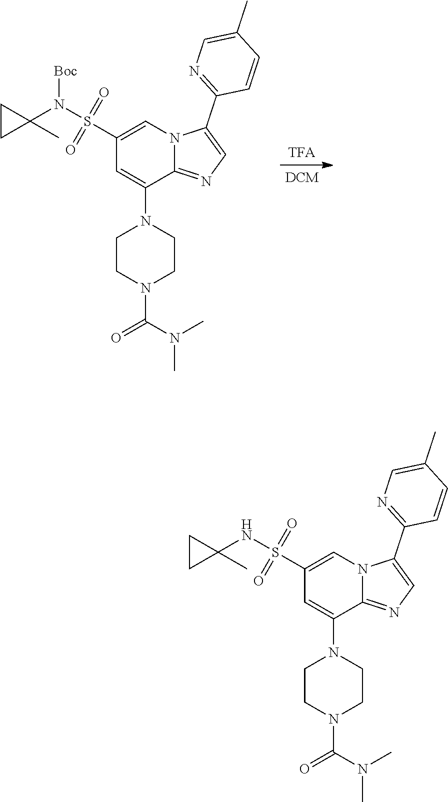 [SMILES: Cc1ccc(-c2cnc3c(N4CCN(C(=O)N(C)C)CC4)cc(S(=O)(=O)N(C(=O)OC(C)(C)C)C4(C)CC4)cn23)nc1 ; Cc1ccc(-c2cnc3c(N4CCN(C(=O)N(C)C)CC4)cc(S(=O)(=O)NC4(C)CC4)cn23)nc1]

To a solution of tert-butyl ((8-(4-(dimethylcarbamoyl)piperazin-1-yl)-3-(5-methylpyridin-2-yl)imidazo[1,2-a]pyridin-6-yl)sulfonyl)(1-methylcyclopropyl)carbamate (12 mg, 20.08 μmol) in DCM (3 mL) was added TFA (1.54 g, 13.51 mmol). The reaction mixture was stirred at 15° C. for 1 h and concentrated under reduced pressure. The residue was purified by prep-HPLC (column: Phenomenex luna C18 150*25 mm*10 μm; mobile phase: A: 0.225% formic acid in water, B: MeCN; B %: 35%-65%, 9 min) to give the product N,N-dimethyl-4-(6-(N-(1-methylcyclopropyl)sulfamoyl)-3-(5-methylpyridin-2-yl)imidazo[1,2-a]pyridin-8-yl)piperazine-1-carboxamide (1.63 mg, 3.26 μmol, 16.24% yield, 99.55% purity) as an off-white solid.

RT 0.692 min (method 1); m/z 498.3 (M+H)⁺ (ESI⁺); ¹H NMR (CDCl₃, 400 MHz): 10.18 (d, J=1.6 Hz, 1H), 8.56 (s, 1H), 8.05 (s, 1H), 7.65 (d, J=8.0 Hz, 1H), 7.60 (dd, J=1.6 Hz, J=8.4 Hz, 1H), 6.81 (d, J=1.2 Hz, 1H), 5.0 (s, 1H), 3.65-3.55 (m, 4H), 3.55-3.48 (m, 4H), 2.90 (s, 6H), 2.40 (s, 3H), 1.33 (s, 3H), 0.91 (t, J=6 Hz, 2H), 0.56 (t, J=6 Hz, 2H).

### Preparation of Example 97

### N,N-dimethyl-4-(6-(N-(1-methylcyclopropyl)sulfamoyl)-3-(6-methylpyridin-3-yl)imidazo[1,2-a]pyridin-8-yl)piperazine-1-carboxamide

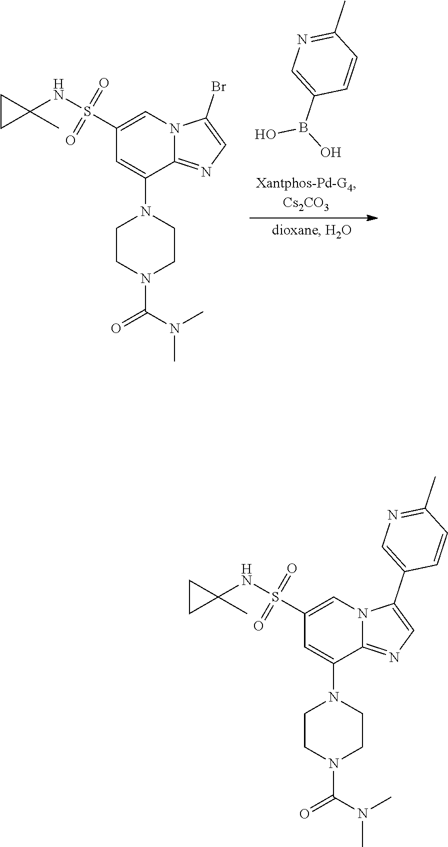

To a solution of 4-(3-bromo-6-(N-(1-methylcyclopropyl)sulfamoyl)imidazo[1,2-a]pyridin-8-yl)-N,N-dimethylpiperazine-1-carboxamide (15 mg, 30.90 μmol), (6-methyl-3-pyridyl)boronic acid (8.46 mg, 61.81 μmol) and Cs₂CO₃ (20.14 mg, 61.81 μmol) in dioxane (1.5 mL) and H₂O (0.3 mL) was added Xantphos Pd G4 (5.95 mg, 6.18 μmol). The reaction mixture was degassed and purged with N₂ (3×), stirred at 80° C. for 16 h and then concentrated under reduced pressure. The resulting residue was purified by preparative-HPLC (column: Phenomenex luna C18 150*25 mm*10 μm; mobile phase: A: 0.225% formic acid in water; B: MeCN; B %: 19%-49%; 10 min) to give the product N,N-dimethyl-4-(6-(N-(1-methylcyclopropyl)sulfamoyl)-3-(6-methylpyridin-3-yl)imidazo[1,2-a]pyridin-8-yl)piperazine-1-carboxamide (6.93 mg, 13.79 μmol, 44.62% yield, 99.01% purity) as a white solid.

RT 0.722 min (method 1); m/z 498.2 (M+H)⁺ (ESI⁺); ¹H NMR (CDCl₃, 400 MHz): 8.69 (s, 1H), 8.42 (s, 1H), 7.75 (d, J=8.0 Hz, 1H), 7.71 (s, 1H), 7.37 (d, J=8.0, 1H), 6.68 (s, 1H), 4.97 (s, 1H), 3.75-3.60 (m, 4H), 3.58-3.48 (m, 4H), 2.90 (s, 6H), 2.69 (s, 3H), 1.30 (s, 3H), 0.82 (t, J=6.0 Hz, 2H), 0.53 (t, J=6.4 Hz, 2H).

### Preparation of Example 98

### 4-(3-ethyl-6-(N-(1-methylcyclopropyl)sulfamoyl)imidazo[1,2-a]pyridin-8-yl)-N,N-dimethylpiperazine-1-carboxamide

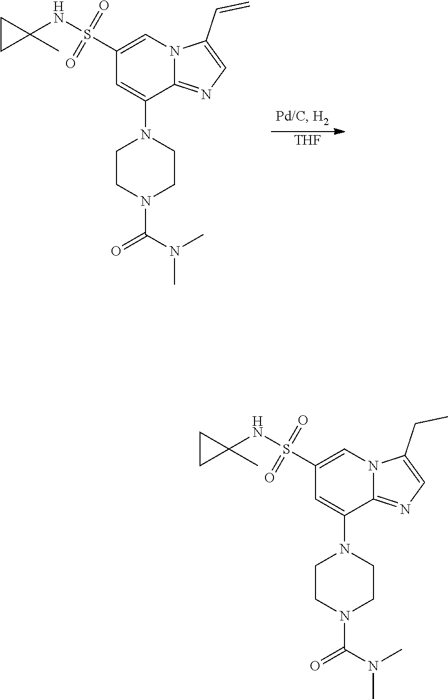 [SMILES: C=Cc1cnc2c(N3CCN(C(=O)N(C)C)CC3)cc(S(=O)(=O)NC3(C)CC3)cn12 ; CCc1cnc2c(N3CCN(C(=O)N(C)C)CC3)cc(S(=O)(=O)NC3(C)CC3)cn12 ; "THF"]

To a solution of N,N-dimethyl-4-(6-(N-(1-methylcyclopropyl)sulfamoyl)-3-vinylimidazo[1,2-a]pyridin-8-yl)piperazine-1-carboxamide (5 mg, 11.56 μmol) in THE (2 mL) was added Pd/C (2 mg, 10% purity) under N₂. The suspension was degassed under vacuum and purged with H₂ (3×). The mixture was stirred under H₂ (15 psi) at 20° C. for 3 h then filtered and the filtrate was concentrated under reduced pressure. The resulting residue was purified by preparative-HPLC (column: Unisil 3-100 C18 Ultra 150*50 mm*3 μm; mobile phase: A: 0.225% formic acid in water; B: MeCN; B %: 18%-48%, 10 min) to give the product 4-(3-ethyl-6-(N-(1-methylcyclopropyl)sulfamoyl)imidazo[1,2-a]pyridin-8-yl)-N,N-dimethylpiperazine-1-carboxamide (2.98 mg, 6.86 μmol, 59.32% yield, 100% purity) as an off-white solid

RT 0.663 min (method 1); m/z 435.2 (M+H)⁺ (ESI⁺); ¹H NMR (CDCl₃, 400 MHz): 8.22 (d, J=1.2 Hz, 1H), 7.42 (s, 1H), 6.61 (d, J=1.2 Hz, 1H), 5.08 (s, 1H), 3.70-3.60 (m, 4H), 3.60-3.47 (m, 4H), 2.95-2.80 (s, 8H), 1.42 (t, J=7.6 Hz, 3H), 1.29 (s, 3H), 0.83 (t, J=5.6 Hz, 2H), 0.52 (t, J=5.2 Hz, 2H).

### Preparation of Example 99

### N,N-dimethyl-4-(3-(3-methylbut-1-yn-1-yl)-6-(N-(1-methylcyclopropyl)sulfamoyl)imidazo[1,2-a]pyridin-8-yl)piperazine-1-carboxamide

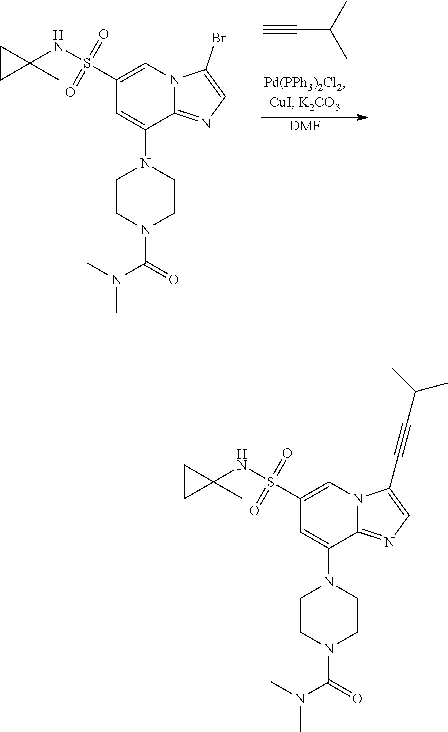

To a solution of 4-(3-bromo-6-(N-(1-methylcyclopropyl)sulfamoyl)imidazo[1,2-a]pyridin-8-yl)-N,N-dimethylpiperazine-1-carboxamide (15 mg, 30.90 μmol) in DMF (1.5 mL) was added CuI (588.54 μg, 3.09 μmol), K₂CO₃ (17.08 mg, 123.61 μmol) and Pd(PPh₃)₂Cl₂ (2.17 mg, 3.09 μmol). The reaction mixture was degassed and purged with N₂ (3×). Then, 3-methylbut-1-yne (2.32 mg, 33.99 μmol, 3.48 μL) was added to the mixture through an injection syringe. The reaction mixture was stirred at 110° C. for 16 h under N₂ atmosphere then poured into water (5 mL) and extracted with EtOAc (5 mL, 3×). The combined organic layer was dried over Na₂SO₄, filtered and concentrated under reduced pressure. The resulting residue was purified by preparative-HPLC (column: Unisil 3-100 C18 Ultra 150*50 mm*3 μm; mobile phase: A: 0.225% formic acid in water; B: MeCN; B %: 48%-78%, 7 min) to give the product N,N-dimethyl-4-(3-(3-methylbut-1-yn-1-yl)-6-(N-(1-methylcyclopropyl)sulfamoyl)imidazo[1,2-a]pyridin-8-yl)piperazine-1-carboxamide (3.35 mg, 7.09 μmol, 22.94% yield, 100% purity) as an off-white solid.

RT 0.849 min (method 1); m/z 473.3 (M+H)⁺ (ESI⁺); ¹H NMR (CDCl₃, 400 MHz): 8.50 (d, J=1.6 Hz, 1H), 7.73 (s, 1H), 6.67 (d, J=1.6 Hz, 1H), 4.93 (s, 1H), 3.70-3.58 (m, 4H), 3.58-3.45 (m, 4H), 3.00-2.92 (m, 1H), 2.89 (s, 6H), 1.36 (d, J=6.8 Hz, 6H), 1.32 (s, 3H), 0.86 (t, J=6.0 Hz, 2H), 0.55 (t, J=5.2 Hz, 2H).

### Preparation of Intermediate 100.1

### N,N-dimethyl-4-(6-(N-(1-methylcyclopropyl)sulfamoyl)-3-((trimethylsilyl)ethynyl)imidazo[1,2-a]pyridin-8-yl)piperazine-1-carboxamide

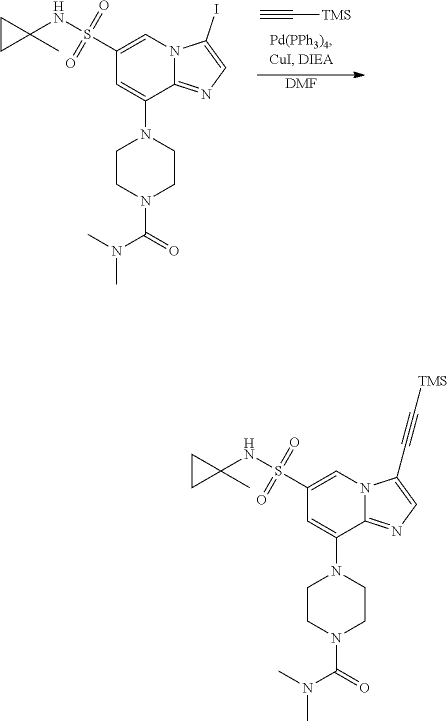

To a solution of 4-(3-iodo-6-(N-(1-methylcyclopropyl)sulfamoyl)imidazo[1,2-a]pyridin-8-yl)-N,N-dimethylpiperazine-1-carboxamide (15 mg, 28.17 μmol) in DMF (2 mL) was added CuI (1.61 mg, 8.45 μmol), DIPEA (5.46 mg, 42.26 μmol, 7.36 μL) and Pd(PPh₃)₄ (1.63 mg, 1.41 μmol). The reaction mixture was degassed and purged with N₂ (3×). Then, ethynyltrimethylsilane (3.32 mg, 33.81 μmol, 4.68 μL) was added to the mixture through an injection syringe. The mixture was stirred at 80° C. for 4 h under N₂ then, poured into water (10 mL) and extracted with EtOAc (10 mL, 3×). The combined organic layer was dried over Na₂SO₄, filtered and concentrated under reduced pressure. The resulting residue was purified by preparative-HPLC (column: Phenomenex luna C18 150*25 mm*10 μm; mobile phase: A: 0.225% formic acid in water; B: MeCN; B %: 54%-84%, 10 min) to give the product N,N-dimethyl-4-(6-(N-(1-methylcyclopropyl)sulfamoyl)-3-((trimethylsilyl)ethynyl)imidazo[1,2-a]pyridin-8-yl)piperazine-1-carboxamide (12 mg, 20.29 μmol, 72.02% yield, 85% purity) as a brown solid.

RT 0.938 min (method 1); m/z 503.3 (M+H)⁺ (ESI⁺); ¹H NMR (CDCl₃, 400 MHz): 8.52 (d, J=1.2 Hz, 1H), 7.84 (s, 1H), 6.73 (s, 1H), 4.96 (s, 1H), 3.70-3.58 (m, 4H), 3.58-3.45 (m, 4H), 2.89 (s, 6H), 1.32 (s, 3H), 0.90-0.80 (m, 2H), 0.60-0.50 (m, 2H), 0.08 (s, 9H).

### Preparation of Example 100

### 4-(3-ethynyl-6-(N-(1-methylcyclopropyl)sulfamoyl)imidazo[1,2-a]pyridin-8-yl)-N,N-dimethylpiperazine-1-carboxamide

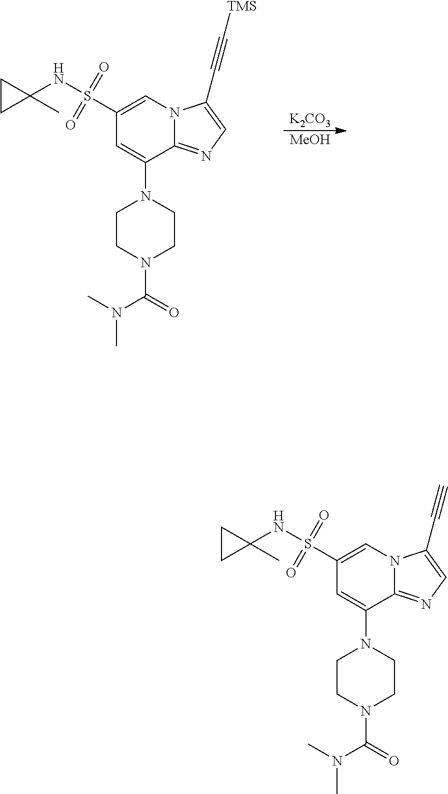

To a solution of N,N-dimethyl-4-(6-(N-(1-methylcyclopropyl)sulfamoyl)-3-((trimethylsilyl)ethynyl)imidazo[1,2-a]pyridin-8-yl)piperazine-1-carboxamide (6 mg, 11.94 μmol) in MeOH (0.5 mL) was added K₂CO₃ (4.95 mg, 35.81 μmol). The reaction mixture was stirred at 20° C. for 2 h. and concentrated under reduced pressure. The resulting residue was purified by preparative-HPLC (column: Waters Xbridge 150*25 mm*5 μm; mobile phase: A: 10 mM aqueous solution of NH₄HCO₃ in water; B: MeCN; B %: 32%-62%; 8 min) to give the product 4-(3-ethynyl-6-(N-(1-methylcyclopropyl)sulfamoyl)imidazo[1,2-a]pyridin-8-yl)-N,N-dimethylpiperazine-1-carboxamide (0.65 mg, 1.51 μmol, 12.65% yield, 100% purity) as off-white gum.

RT 0.814 min (method 1); m/z 431.2 (M+H)⁺ (ESI⁺); ¹H NMR (CDCl₃, 400 MHz): 8.56 (d, J=1.2 Hz, 1H), 7.86 (s, 1H), 6.72 (s, 1H), 4.98 (s, 1H), 3.85 (s, 1H), 3.70-3.60 (m, 4H), 3.58-3.45 (m, 4H), 2.89 (s, 6H), 1.32 (s, 3H), 0.85 (t, J=5.6 Hz, 2H), 0.56 (t, J=6.0 Hz, 2H).

### Preparation of Example 101

### N,N-dimethyl-4-(6-(N-(1-methylcyclopropyl)sulfamoyl)-3-(prop-1-yn-1-yl)imidazo[1,2-a]pyridin-8-yl)piperazine-1-carboxamide

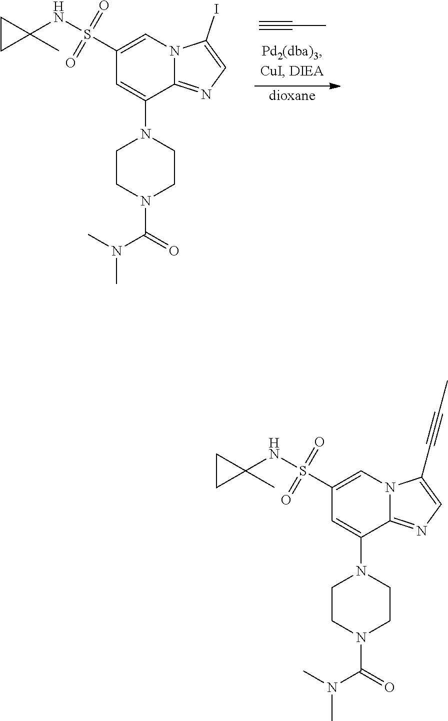

To a solution of 4-(3-iodo-6-(N-(1-methylcyclopropyl)sulfamoyl)imidazo[1,2-a]pyridin-8-yl)-N,N-dimethylpiperazine-1-carboxamide (20 mg, 37.57 μmol) in dioxane (2 mL) was added CuI (715.44 ug, 3.76 μmol), TEA (727.00 mg, 7.18 mmol) and Pd₂(dba)₃ (3.44 mg, 3.76 μmol). The reaction mixture was degassed and purged with N₂ (3×). Then, prop-1-yne (1 M in THF, 45.08 μL) was added. The reaction mixture was stirred at 20° C. for 16 h under N₂, poured into water (10 mL) and extracted with EA (10 mL, 3×). The combined organic layer was dried over Na₂SO₄, filtered and concentrated under reduced pressure. The residue was purified by preparative-HPLC (column: Phenomenex luna C18 150*25 mm*10 μm; mobile phase: A: 0.225% formic acid in water; B: MeCN; B %: 42%-72%; 10 min) to give the product N,N-dimethyl-4-(6-(N-(1-methylcyclopropyl)sulfamoyl)-3-(prop-1-yn-1-yl)imidazo[1,2-a]pyridin-8-yl)piperazine-1-carboxamide (15 mg, 33.74 μmol, 89.82% yield, 100% purity) as a white solid.

RT 0.825 min (method 1); m/z 445.1 (M+H)⁺ (ESI⁺); ¹H NMR (CDCl₃, 400 MHz): 8.54 (d, J=1.2 Hz, 1H), 7.73 (s, 1H), 6.67 (d, J=1.2 Hz, 1H), 5.01 (s, 1H), 3.70-3.55 (m, 4H), 3.55-3.45 (m, 4H), 2.89 (s, 6H), 2.24 (s, 3H), 1.31 (s, 3H), 0.84 (t, J=6.0 Hz, 2H), 0.55 (t, J=6.4 Hz, 2H).

### Preparation of Example 102

### N,N-dimethyl-4-(6-(N-(1-methylcyclopropyl)sulfamoyl)-3-propylimidazo[1,2-a]pyridin-8-yl)piperazine-1-carboxamide

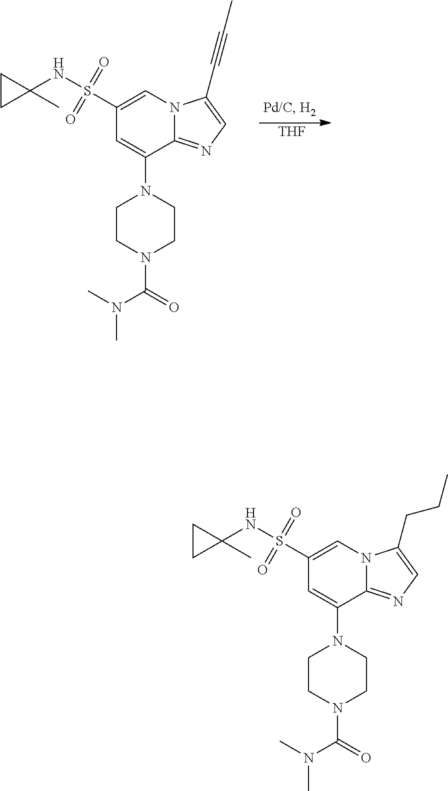 [SMILES: CC#Cc1cnc2c(N3CCN(C(=O)N(C)C)CC3)cc(S(=O)(=O)NC3(C)CC3)cn12 ; CCCc1cnc2c(N3CCN(C(=O)N(C)C)CC3)cc(S(=O)(=O)NC3(C)CC3)cn12 ; "THF"]

To a solution of N,N-dimethyl-4-(6-(N-(1-methylcyclopropyl)sulfamoyl)-3-(prop-1-yn-1-yl)imidazo[1,2-a]pyridin-8-yl)piperazine-1-carboxamide (8 mg, 18.00 μmol) in THF (2 mL) was added Pd/C (4 mg, 10% purity) under N₂. The suspension was degassed under vacuum and purged with H₂ (3×). The mixture was stirred under H₂ (15 psi) at 20° C. for 3 hours, then filtered and the filtrate was concentrated under reduced pressure. The resulting residue was purified by preparative-HPLC (column: Phenomenex luna C18 150*25 mm*10 μm; mobile phase: A: 0.225% formic acid in water; B: MeCN; B %: 25%-55%; 10 min) to give the product N,N-dimethyl-4-(6-(N-(1-methylcyclopropyl)sulfamoyl)-3-propylimidazo[1,2-a]pyridin-8-yl)piperazine-1-carboxamide (4.05 mg, 9.03 μmol, 50.17% yield, 100% purity) as a off-white solid.

RT 0.746 min (method 1); m/z 449.1 (M+H)⁺ (ESI⁺); ¹H NMR (CDCl₃, 400 MHz): 8.23 (s, 1H), 7.42 (s, 1H), 6.60 (s, 1H), 5.00 (s, 1H), 3.70-3.55 (m, 4H), 3.55-3.45 (m, 4H), 2.89 (s, 6H), 2.84 (t, J=7.2 Hz, 2H), 1.90-1.75 (m, 2H), 1.29 (s, 3H), 1.06 (t, J=7.6 Hz, 3H), 0.84 (t, J=6.0 Hz, 2H), 0.52 (t, J=6.4 Hz, 2H).

### Preparation of Example 103

### N,N-dimethyl-4-(6-(N-(1-methylcyclopropyl)sulfamoyl)-3-(trifluoromethyl)imidazo[1,2-a]pyridin-8-yl)piperazine-1-carboxamide

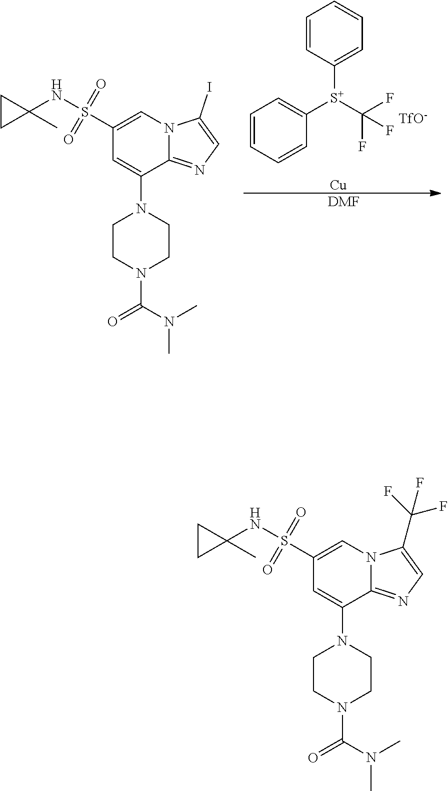

To a solution of 4-(3-iodo-6-(N-(1-methylcyclopropyl)sulfamoyl)imidazo[1,2-a]pyridin-8-yl)-N,N-dimethylpiperazine-1-carboxamide (6 mg, 11.27 μmol) in DMF (1 mL) was added diphenyl(trifluoromethyl)sulfonium; trifluoromethanesulfonate (6.84 mg, 16.90 μmol) and copper (1.43 mg, 22.54 μmol). The reaction mixture was stirred at 60° C. for 2 h under N₂, then, s filtered and the filtrate was concentrated under reduced pressure. The resulting residue was purified by preparative HPLC (column: Unisil 3-100 C18 Ultra 150*50 mm*3 μm; mobile phase: A: 0.225% formic acid in water; B: MeCN; B %: 41%-71%; 10 min) to give the product N,N-dimethyl-4-(6-(N-(1-methylcyclopropyl)sulfamoyl)-3-(trifluoromethyl)imidazo[1,2-a]pyridin-8-yl)piperazine-1-carboxamide (1.55 mg, 3.27 μmol, 28.99% yield, 100% purity) as yellow gum.

RT 0.908 min (method 1); m/z 475.2 (M+H)⁺ (ESI⁺); ¹H NMR (CDCl₃, 400 MHz): 8.44 (s, 1H), 7.96 (s, 1H), 6.80 (s, 1H), 5.00 (s, 1H), 3.70-3.60 (m, 4H), 3.60-3.45 (m, 4H), 2.90 (s, 6H), 1.33 (s, 3H), 0.84 (t, J=6.0 Hz, 2H), 0.57 (t, J=6.0 Hz, 2H).

Compounds listed in the table below were prepared according to the corresponding general procedures or, when stated in a similar way to the related example, and starting from the corresponding intermediates or examples.

| Cpd | | Yield | | | |
|---|---|---|---|---|---|
| number | Structure | (%) | Procedure | LC/MS | 1H NMR |
| 105 | 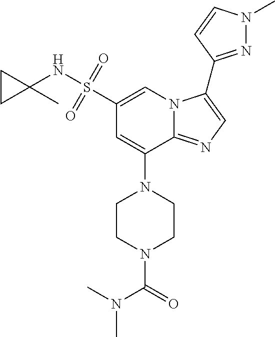 [SMILES: CN(C)C(=O)N1CCN(c2cc(S(=O)(=O)NC3(C)CC3)cn3c(-c4ccn(C)n4)cnc23)CC1] | 27.6 | Procedure 6 | RT 0.742 min (method 1); m/z 487.2 (M + H)⁺ (ESI⁺); | ¹H NMR (CDCl₃, 400 MHz): 9.66 (s, 1H), 7.86 (s, 1H), 7.47 (s, 1H), 6.75 (s, 1H), 6.59 (s, 1H), 4.94 (s, 1H), 4.03 (s, 3H), 3.75-3.60 (m, 4H), 3.58-3.48 (m, 4H), 2.90 (s, 6H), 1.32 (s, 3H), 0.90 (t, J = 6.0 Hz, 2H), 0.55 (t, J = 6.4 Hz, 2H) |
| 106 | 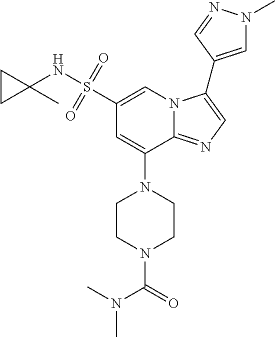 [SMILES: CN(C)C(=O)N1CCN(c2cc(S(=O)(=O)NC3(C)CC3)cn3c(-c4cnn(C)c4)cnc23)CC1] | 39.44 | Procedure 6 | RT 0.708 min (method 1); m/z 487.3 (M + H)⁺ (ESI⁺); | ¹H NMR (CDCl₃, 400 MHz): 8.40 (d, J = 1.6 Hz, 1H), 7.72 (s, 1H), 7.66 (s, 1H), 7.62 (s, 1H), 6.64 (d, J = 1.6 Hz, 1H), 4.92 (s, 1H), 4.04 (s, 3H), 3.75-3.60 (m, 4H), 3.58- 3.48 (m, 4H), 2.90 (s, 6H), 1.30 (s, 3H), 0.82 (t, J = 6.4 Hz, 2H), 0.53 (t, J = 6.4 Hz, 2H) |
| 107 | 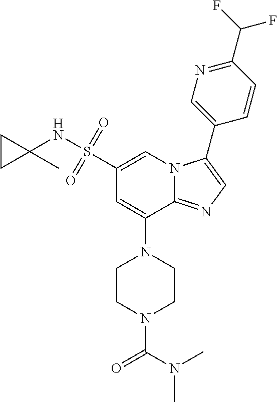 [SMILES: CN(C)C(=O)N1CCN(c2cc(S(=O)(=O)NC3(C)CC3)cn3c(-c4ccc(C(F)F)nc4)cnc23)CC1] | 55.38 | Procedure 6 | RT 0.828 min (method 1); m/z 534.2 (M + H)⁺ (ESI⁺); | ¹H NMR (CDCl₃, 400 MHz): 8.86 (s, 1H), 8.46 (s, 1H), 8.04 (dd, J = 1.6 Hz, 8.0 Hz, 1H), 7.85 (d, J = 8.0 Hz, 1H), 7.80 (s, 1H), 6.74 (t, J = 55.2 Hz, 1H), 6.72 (s, 1H), 5.02 (s, 1H), 3.75-3.60 (m, 4H), 3.60- 3.50 (m, 4H), 2.90 (s, 6H), 1.31 (s, 3H), 0.82 (t, J = 6.0 Hz, 2H), 0.55 (t, J = 5.2 Hz, 2H) |
| 108 | 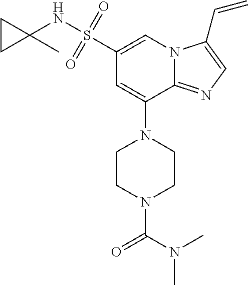 [SMILES: C=Cc1cnc2c(N3CCN(C(=O)N(C)C)CC3)cc(S(=O)(=O)NC3(C)CC3)cn12] | 80.01 | Procedure 6 | RT 0.760 min (method 1); m/z 433.1 (M + H)⁺ (ESI⁺); | ¹H NMR (CDCl₃, 400 MHz): 8.42 (d, J = 1.2 Hz, 1H), 7.7 (s, 1H), 6.82 (dd, J = 1.6 Hz, 11.6 Hz, 1H), 6.65 (s, 1H), 5.85 (d, J = 17.6 Hz, 1H), 5.49 (d, J = 12.0 Hz, 1H), 5.02 (s, 1H), 3.70-3.60 (m, 4H), 3.60-3.47 (m, 4H), 2.89 (s, 6H), 1.30 (s, 3H), 0.83 (t, J = 6.0 Hz, 2H), 0.54 (t, J = 5.2 Hz, 2H) |
| 109 | 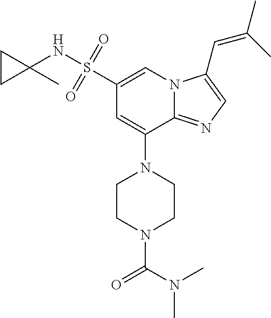 [SMILES: CC(C)=Cc1cnc2c(N3CCN(C(=O)N(C)C)CC3)cc(S(=O)(=O)NC3(C)CC3)cn12] | 72.56 | Procedure 6, followed by Procedure 2 | RT 0.776 min (method 1); m/z 461.2 (M + H)⁺ (ESI⁺); | ¹H NMR (CDCl₃, 400 MHz): 8.30 (d, J = 1.2 Hz, 1H), 7.55 (s, 1H), 6.62 (s, 1H), 6.17 (s, 1H), 4.95 (s, 1H), 3.70-3.60 (m, 4H), 3.60- 3.47 (m, 4H), 2.89 (s, 6H), 2.05 (s, 3H), 1.94 (s, 3H) 1.30 (s, 3H), 0.84 (t, J = 5.2 Hz, 2H), 0.53 (t, J = 5.2 Hz, 2H) |
| 110 | 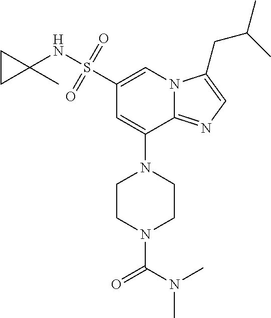 [SMILES: CC(C)Cc1cnc2c(N3CCN(C(=O)N(C)C)CC3)cc(S(=O)(=O)NC3(C)CC3)cn12] | 72.56 | Procedure 7 | RT 0.767 min (method 1); m/z 463.1 (M + H)⁺ (ESI⁺); | ¹H NMR (CDCl₃, 400 MHz): 8.24 (s, 1H), 7.42 (s, 1H), 6.60 (s, 1H), 4.99 (s, 1H), 3.65-3.56 (m, 4H), 3.56-3.45 (m, 4H), 2.89 (s, 6H), 2.76 (d, J = 7.2 Hz, 2H), 2.15- 2.00 (m, 1H), 1.29 (s, 3H), 1.01 (d, J = 6.4 Hz, 6H), 0.83 (t, J = 5.6 Hz, 2H), 0.52 (t, J = 5.2 Hz, 2H) |
| 111 | 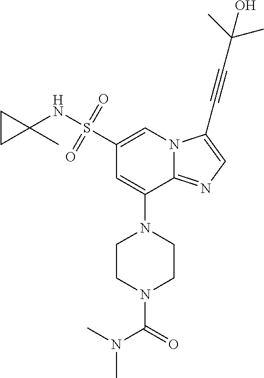 [SMILES: CN(C)C(=O)N1CCN(c2cc(S(=O)(=O)NC3(C)CC3)cn3c(C#CC(C)(C)O)cnc23)CC1] | 30.48 | Procedure 8 | RT 0.815 min (method 1); m/z 489.2 (M + H)⁺ (ESI⁺); | ¹H NMR (CDCl₃, 400 MHz): 8.47 (d, J = 1.6 Hz, 1H), 7.80 (s, 1H), 6.71 (s, 1H), 5.04 (s, 1H), 3.70-3.58 (m, 4H), 3.58- 3.45 (m, 4H), 2.89 (s, 6H), 2.23 (s, 1H), 1.72 (s, 6H), 1.32 (s, 3H), 0.85 (t, J = 6.0 Hz, 2H), 0.56 (t, J = 5.6 Hz, 2H) |

### Alternative Preparation of Intermediate 10.7

### 2-(6-(benzylthio)-8-chloro-1-iodoindolizin-3-yl)-5-(difluoromethyl)-1,3,4-thiadiazole

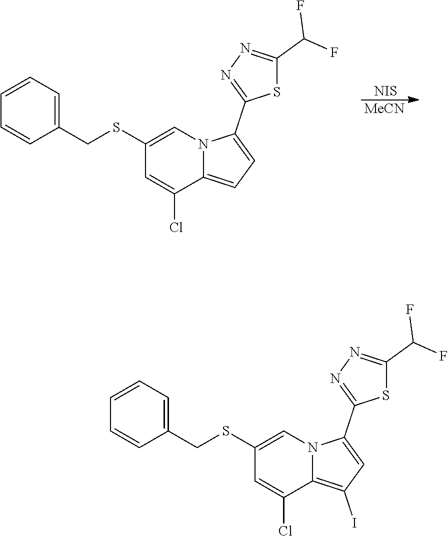

To a solution of 2-(6-(benzylthio)-8-chloroindolizin-3-yl)-5-(difluoromethyl)-1,3,4-thiadiazole (700 mg, 1.72 mmol) in ACN (10 mL) was added NIS (424.72 mg, 1.89 mmol). The mixture was stirred at 50° C. for 2 h and concentrated under vacuum. The crude product was triturated with MeOH(10 mL) at 20° C. for 5 min and the precipitate was filtered. The cake was dried under vacuum to give the product 2-(6-(benzylthio)-8-chloro-1-iodoindolizin-3-yl)-5-(difluoromethyl)-1,3,4-thiadiazole (700 mg, 1.31 mmol, 76.41% yield) as a yellow solid.

RT 0.812 min (method 3); m/z 533.8 (M+H)⁺ (ESI⁺).

### Preparation of Intermediate 113.1

### 8-chloro-3-(5-(difluoromethyl)-1,3,4-thiadiazol-2-yl)-1-iodoindolizine-6-sulfonyl chloride & 1,8-dichloro-3-(5-(difluoromethyl)-1,3,4-thiadiazol-2-yl)indolizine-6-sulfonyl chloride

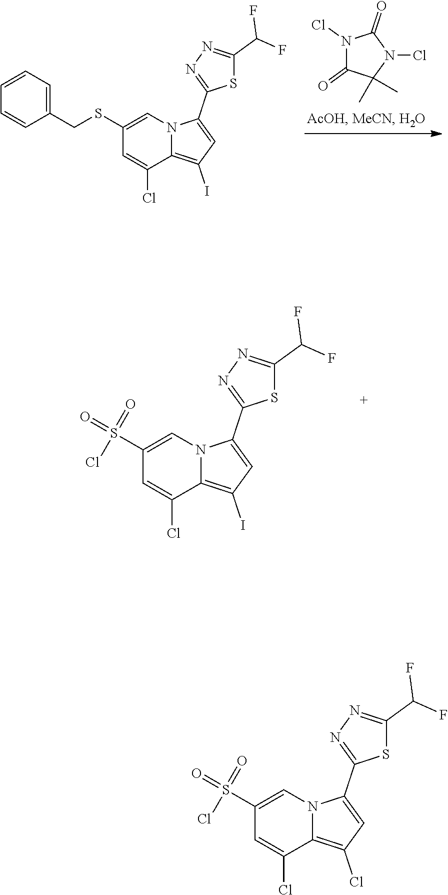 [SMILES: FC(F)c1nnc(-c2cc(I)c3c(Cl)cc(SCc4ccccc4)cn23)s1 ; CC1(C)C(=O)N(Cl)C(=O)N1Cl ; O=S(=O)(Cl)c1cc(Cl)c2c(I)cc(-c3nnc(C(F)F)s3)n2c1 ; O=S(=O)(Cl)c1cc(Cl)c2c(Cl)cc(-c3nnc(C(F)F)s3)n2c1]

To a solution of 2-(6-(benzylthio)-8-chloro-1-iodoindolizin-3-yl)-5-(difluoromethyl)-1,3,4-thiadiazole (250 mg, 468.35 μmol), AcOH (112.50 mg, 1.87 mmol, 107.15 μL) and H₂O (33.75 mg, 1.87 mmol, 33.75 μL) in ACN (3 mL) was added 1,3-dichloro-5,5-dimethyl-imidazolidine-2,4-dione (276.82 mg, 1.41 mmol) at 0° C. The reaction was stirred at 0° C. for 15 min affording a mixture of 8-chloro-3-(5-(difluoromethyl)-1,3,4-thiadiazol-2-yl)-1-iodoindolizine-6-sulfonyl chloride & 1,8-dichloro-3-(5-(difluoromethyl)-1,3,4-thiadiazol-2-yl)indolizine-6-sulfonyl chloride which was used directly as such in the next step.

### Preparation of Intermediate 113.2

### 8-chloro-N-(1-cyanocyclopropyl)-3-(5-(difluoromethyl)-1,3,4-thiadiazol-2-yl)-1-iodoindolizine-6-sulfonamide & 1,8-dichloro-N-(1-cyanocyclopropyl)-3-(5-(difluoromethyl)-1,3,4-thiadiazol-2-yl)indolizine-6-sulfonamide

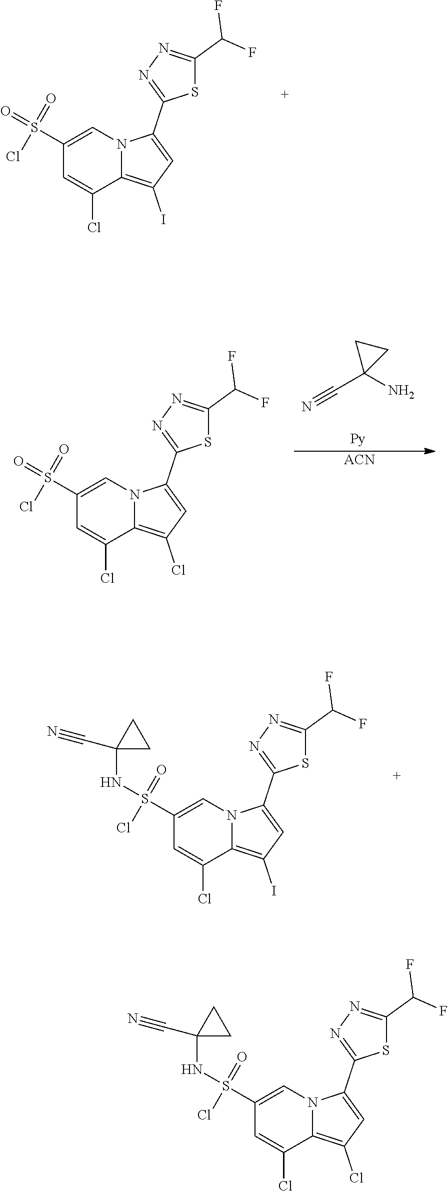

The mixture of 8-chloro-3-(5-(difluoromethyl)-1,3,4-thiadiazol-2-yl)-1-iodoindolizine-6-sulfonyl chloride & 1,8-dichloro-3-(5-(difluoromethyl)-1,3,4-thiadiazol-2-yl)indolizine-6-sulfonyl chloride was added dropwise to a solution of 1-aminocyclopropanecarbonitrile hydrochloride (165.96 mg, 1.40 mmol) in Py (1 mL) and MeCN (3 mL) at 0° C. The mixture was allowed to warm to 20° C., stirred for 1 h and poured into a HCl solution (1 N, 30 mL). The aqueous phase was extracted with ethyl acetate (10 mL, 2×). The combined organic phases were washed with brine (20 mL), dried with anhydrous Na₂SO₄, filtered and concentrated in vacuum. The residue was purified by flash silica gel chromatography (ISCO®; 4 g SepaFlash® Silica Flash Column, Eluent of 0˜25% Ethyl acetate/Petroleum ether gradient @12 mL/min) to give the product 8-chloro-N-(1-cyanocyclopropyl)-3-(5-(difluoromethyl)-1,3,4-thiadiazol-2-yl)-1-iodoindolizine-6-sulfonamide & 1,8-dichloro-N-(1-cyanocyclopropyl)-3-(5-(difluoromethyl)-1,3,4-thiadiazol-2-yl)indolizine-6-sulfonamide (130 mg, crude) as a yellow solid.

RT 0.890 min (method 1); m/z 555.9 & 463.9 (M+H)⁺ (ESI⁺).

### Preparation of Example 113 and Example 114

### 4-(6-(N-(1-cyanocyclopropyl)sulfamoyl)-3-(5-(difluoromethyl)-1,3,4-thiadiazol-2-yl)indolizin-8-yl)-N,N-dimethylpiperazine-1-carboxamide & 4-(1-chloro-6-(N-(1-cyanocyclopropyl)sulfamoyl)-3-(5-(difluoromethyl)-1,3,4-thiadiazol-2-yl)indolizin-8-yl)-N,N-dimethylpiperazine-1-carboxamide

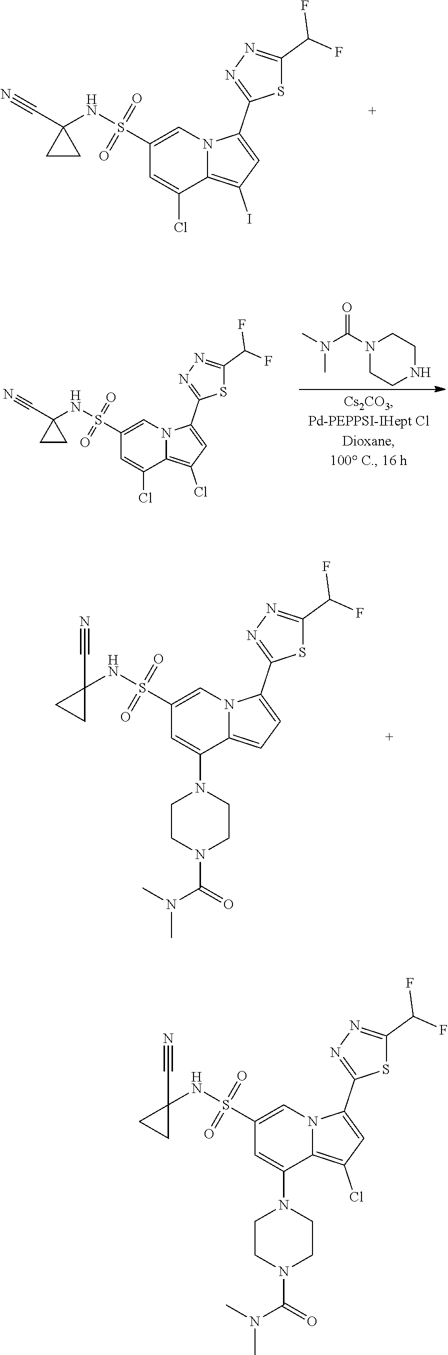

A mixture of 8-chloro-N-(1-cyanocyclopropyl)-3-[5-(difluoromethyl)-1,3,4-thiadiazol-2-yl]-1-iodo-indolizine-6-sulfonamide and 1,8-dichloro-N-(1-cyanocyclopropyl)-3-(5-(difluoromethyl)-1,3,4-thiadiazol-2-yl)indolizine-6-sulfonamide (100 mg, crude), N,N-dimethylpiperazine-1-carboxamide (31.12 mg, 197.93 μmol), Cs₂CO₃ (117.25 mg, 359.88 μmol) and Pd-PEPPSI-IPentCl o-picoline (17.50 mg, 17.99 μmol) in dioxane (1 mL) was degassed and purged with Ar₂ (3×) in glove box. Then, the mixture was stirred at 80° C. for 26 h under Ar atmosphere outside of the glove box. The mixture was filtered and the mother solution was concentrated in vacuum. The resulting residue was purified by preparative TLC (SiO₂, Petroleum ether:Ethyl acetate=1:1) to give two impure products.

The impure product 1 was re-purified by preparative TLC (SiO₂, Petroleum ether:Ethyl acetate=0:1) and lyophilized directly to give the product 4-(6-(N-(1-cyanocyclopropyl)sulfamoyl)-3-(5-(difluoromethyl)-1,3,4-thiadiazol-2-yl)indolizin-8-yl)-N,N-dimethylpiperazine-1-carboxamide (2.3 mg, 4.12 μmol, 2.29% yield, 98.511% purity) as a yellow solid.

RT 0.585 min (method 3); m/z 551.1 (M+H)⁺ (ESI⁺); ¹H NMR (DMSO-d₆, 400 MHz): 9.97 (s, 1H), 9.32 (br, 1H), 7.84 (d, J=4.4 Hz, 1H), 7.52-7.79 (m, 1H), 6.96 (d, J=4.8 Hz, 1H), 6.79 (s, 1H), 3.36-3.38 (m, 4H), 3.22 (s, 4H), 2.80 (s, 6H), 1.44-1.49 (m, 2H), 1.35-1.40 (m, 2H)

The impure product 2 was purified by preparative HPLC (column: Phenomenex luna C18 150*25 mm*10 μm; mobile phase: A: 0.225% formic acid in water, B: MeCN; B %: 42%-72%, 10 min), followed by preparative HPLC (column: Waters Xbridge 150*25 mm*10 μm; mobile phase: A: 1 mM aqueous solution of NH₄HCO₃, B: MeCN; B %: 35%-65%, 11 min) and lyophilized directly to give the product 4-(1-chloro-6-(N-(1-cyanocyclopropyl)sulfamoyl)-3-(5-(difluoromethyl)-1,3,4-thiadiazol-2-yl)indolizin-8-yl)-N,N-dimethylpiperazine-1-carboxamide (1.73 mg, 2.96 μmol, 1.64% yield, 99.99% purity) as a yellow solid.

RT 0.587 min (method 3); m/z 585.0 (M+H)⁺ (ESI⁺); ¹H NMR (DMSO-d₆, 400 MHz): 9.98 (s, 1H), 9.06-9.19 (m, 1H), 7.90 (s, 1H), 7.46-7.74 (m, 1H), 6.91 (s, 1H), 3.41 (s, 4H), 3.08-3.12 (m, 4H), 2.82 (s, 6H), 1.44-1.46 (m, 2H), 1.39-1.41 (m, 2H).

### Preparation of Example 115

### 8-chloro-3-(5-(difluoromethyl)-1,3,4-thiadiazol-2-yl)-1-iodo-N-(1-methylcyclopropyl)indolizine-6-sulfonamide

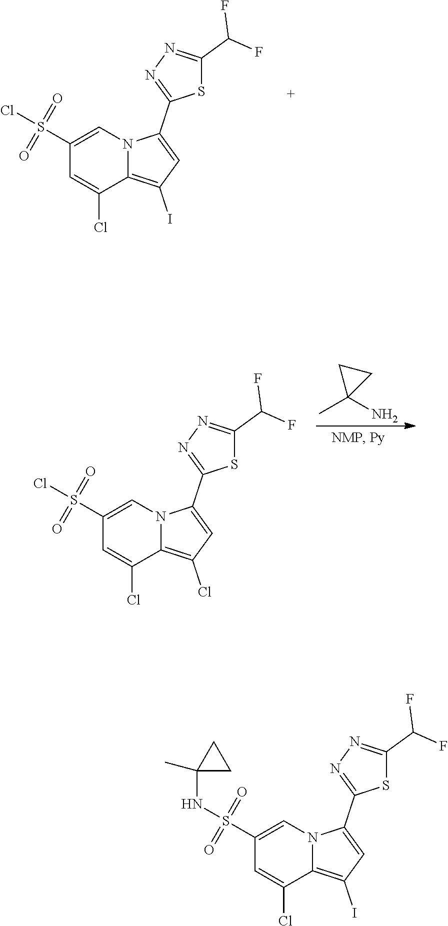

To a solution of 1-methylcyclopropanamine (30.05 mg, 279.35 μmol, HCl salt) in NMP (0.5 mL) and Pyridine (0.5 mL) was added the crude mixture of 8-chloro-3-(5-(difluoromethyl)-1,3,4-thiadiazol-2-yl)-1-iodoindolizine-6-sulfonyl chloride & 1,8-dichloro-3-(5-(difluoromethyl)-1,3,4-thiadiazol-2-yl)indolizine-6-sulfonyl chloride (95 mg, crude) at 0° C. The reaction was stirred at 20° C. for 2 h and concentrated under vacuum. The residue was purified by preparative HPLC (column: Phenomenex luna C18 150*25 mm*10 μm; mobile phase: A: 0.225% formic acid in water, B: MeCN; B %: 61%-91%, 10 min) and lyophilized directly to give the product 8-chloro-3-(5-(difluoromethyl)-1,3,4-thiadiazol-2-yl)-1-iodo-N-(1-methylcyclopropyl)indolizine-6-sulfonamide (25 mg, 45.89 μmol, 24.64% yield) as a brown solid.

RT 0.668 min (method 3); m/z 544.8 (M+H)⁺ (ESI⁺); ¹H NMR (DMSO-d₆, 400 MHz): 10.19 (d, J=1.2 Hz, 1H), 8.45 (s, 1H), 8.18 (s, 1H), 7.82-7.66 (m, 1H), 7.43 (d, J=1.2 Hz, 1H), 1.18-1.16 (m, 3H), 0.73-0.68 (m, 2H), 0.48-0.43 (m, 2H).

### Preparation of Example 116

### 8-chloro-3-(5-(difluoromethyl)-1,3,4-thiadiazol-2-yl)-N-(1-methylcyclopropyl)indolizine-6-sulfonamide

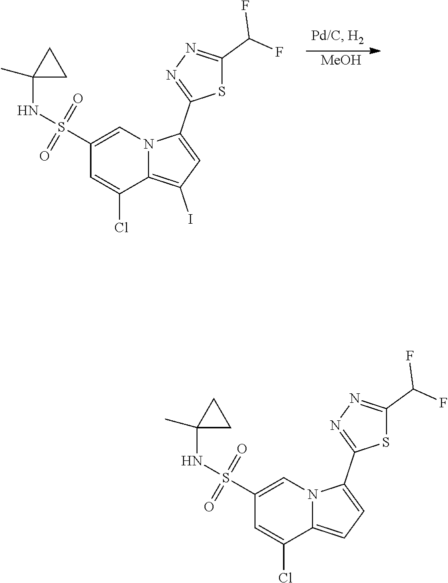 [SMILES: "MeOH" ; CC1(NS(=O)(=O)c2cc(Cl)c3c(I)cc(-c4nnc(C(F)F)s4)n3c2)CC1 ; CC1(NS(=O)(=O)c2cc(Cl)c3ccc(-c4nnc(C(F)F)s4)n3c2)CC1]

To a solution of 8-chloro-3-(5-(difluoromethyl)-1,3,4-thiadiazol-2-yl)-1-iodo-N-(1-methylcyclopropyl)indolizine-6-sulfonamide (23 mg, 42.22 μmol) in MeOH (0.5 mL) was added Pd/C (5 mg, 10% purity). The mixture was degassed, purged with H₂ (3×) and stirred at 20° C. for 16 h under H₂ (15 Psi). The mixture was filtered to remove the insoluble and the filtrate was concentrated under vacuum to give the product 8-chloro-3-(5-(difluoromethyl)-1,3,4-thiadiazol-2-yl)-N-(1-methylcyclopropyl)indolizine-6-sulfonamide (10 mg, 23.87 μmol, 56.55% yield) as a gray solid.

RT 0.625 min (method 3); m/z 418.9 (M+H)⁺ (ESI⁺).

### Preparation of Example 117

### 4-(3-(5-(difluoromethyl)-1,3,4-thiadiazol-2-yl)-6-(N-(1-methylcyclopropyl)sulfamoyl)indolizin-8-yl)-N,N-dimethylpiperazine-1-carboxamide

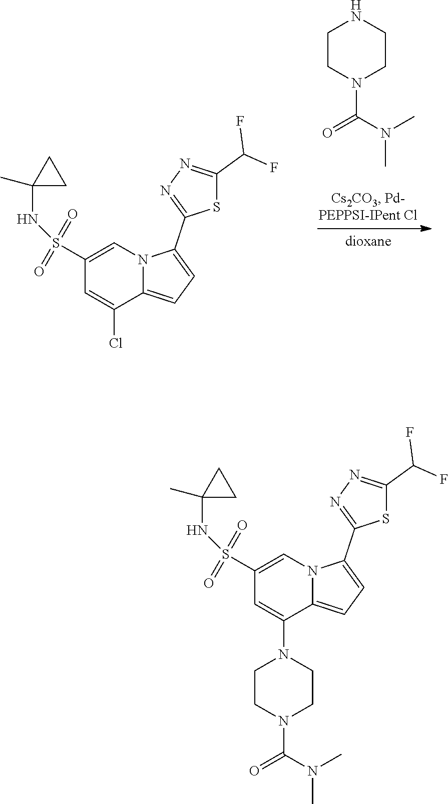 [SMILES: CC1(NS(=O)(=O)c2cc(Cl)c3ccc(-c4nnc(C(F)F)s4)n3c2)CC1 ; CN(C)C(=O)N1CCNCC1 ; CN(C)C(=O)N1CCN(c2cc(S(=O)(=O)NC3(C)CC3)cn3c(-c4nnc(C(F)F)s4)ccc23)CC1]

A mixture of N,N-dimethylpiperazine-1-carboxamide (7.51 mg, 47.75 μmol), 8-chloro-3-(5-(difluoromethyl)-1,3,4-thiadiazol-2-yl)-N-(1-methylcyclopropyl)indolizine-6-sulfonamide (10 mg, 23.87 μmol), Cs₂CO₃ (15.56 mg, 47.75 μmol) and Pd-PEPPSI-IPentCl o-picoline (1 mg, 1.03 μmol) in dioxane (0.5 mL) was degassed and purged with Ar (3×) in a glove box. Then, the mixture was stirred at 100° C. for 16 h under Ar atmosphere outside of the glove box, and concentrated under vacuum. The residue was purified by preparative HPLC (column: Phenomenex Luna C18 100*30 mm*5 μm; mobile phase: A: 0.225% formic acid in water, B: MeCN; B %: 40%-70%, 8 min) and lyophilized directly to give the product 4-(3-(5-(difluoromethyl)-1,3,4-thiadiazol-2-yl)-6-(N-(1-methylcyclopropyl)sulfamoyl)indolizin-8-yl)-N,N-dimethylpiperazine-1-carboxamide (1.28 mg, 2.32 μmol, 9.74% yield, 98% purity) as a yellow solid.

RT 0.575 min (method 3); m/z 540.1 (M+H)⁺ (ESI⁺); ¹H NMR (DMSO-d₆, 400 MHz): 9.89 (s, 1H), 8.27 (s, 1H), 7.81 (d, J=4.8 Hz, 1H), 7.79-7.50 (m, 1H), 6.93 (d, J=4.4 Hz, 1H), 6.79 (s, 1H), 3.39-3.35 (m, 4H), 3.19 (br s, 4H), 2.80 (s, 6H), 1.13 (s, 3H), 0.70 (s, 2H), 0.43 (d, J=1.6 Hz, 2H).

### Preparation of Example 118

### 1,8-dichloro-3-(5-(difluoromethyl)-1,3,4-thiadiazol-2-yl)-N-(1-(fluoromethyl)cyclopropyl)indolizine-6-sulfonamide

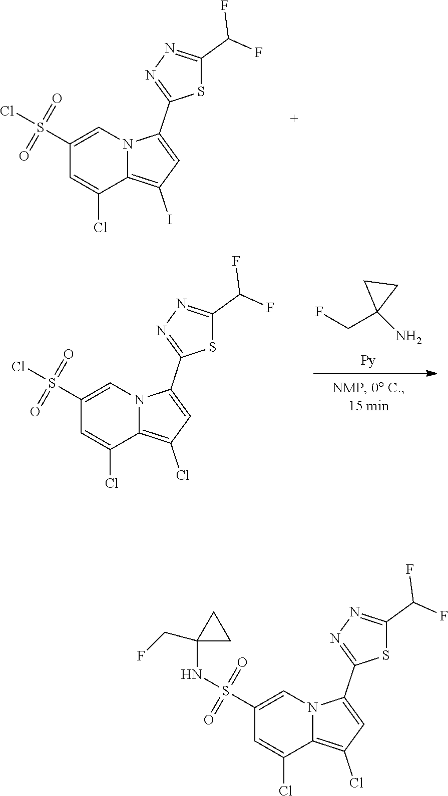 [SMILES: O=S(=O)(Cl)c1cc(Cl)c2c(I)cc(-c3nnc(C(F)F)s3)n2c1 ; O=S(=O)(Cl)c1cc(Cl)c2c(Cl)cc(-c3nnc(C(F)F)s3)n2c1 ; NC1(CF)CC1 ; O=S(=O)(NC1(CF)CC1)c1cc(Cl)c2c(Cl)cc(-c3nnc(C(F)F)s3)n2c1]

To a solution of 1-(fluoromethyl)cyclopropanamine (45.26 mg, 360.47 μmol, HCl salt) in Pyridine (1 mL) and NMP (0.5 mL) was added the crude mixture of 8-chloro-3-(5-(difluoromethyl)-1,3,4-thiadiazol-2-yl)-1-iodoindolizine-6-sulfonyl chloride & 1,8-dichloro-3-(5-(difluoromethyl)-1,3,4-thiadiazol-2-yl)indolizine-6-sulfonyl chloride (95 mg, crude) at 0° C. The mixture was stirred at 20° C. for 2 hand concentrated under vacuum. The residue was purified by preparative HPLC (column: Phenomenex luna C18 150*25 mm*10 μm; mobile phase: A: 0.225% formic acid in water, B: MeCN; B %: 57%-58%, 10 min) and lyophilized directly to give the product 1,8-dichloro-3-(5-(difluoromethyl)-1,3,4-thiadiazol-2-yl)-N-(1-(fluoromethyl)cyclopropyl)indolizine-6-sulfonamide (25 mg, 53.04 μmol, 28.48% yield) as a brown solid.

RT 0.633 min (method 3); m/z 472.8 (M+H)⁺ (ESI⁺); ¹H NMR (DMSO-d₆, 400 MHz): 10.18 (d, J=1.2 Hz, 1H), 8.85 (s, 1H), 8.19 (s, 1H), 7.83-7.53 (m, 1H), 7.45 (d, J=1.2 Hz, 1H), 4.32-4.16 (m, 2H), 0.88-0.84 (m, 2H), 0.81-0.77 (m, 2H).

### Preparation of Example 119

### 4-(3-(5-(difluoromethyl)-1,3,4-thiadiazol-2-yl)-6-(N-(1-(fluoromethyl)cyclopropyl)sulfamoyl)indolizin-8-yl)-N,N-dimethylpiperazine-1-carboxamide

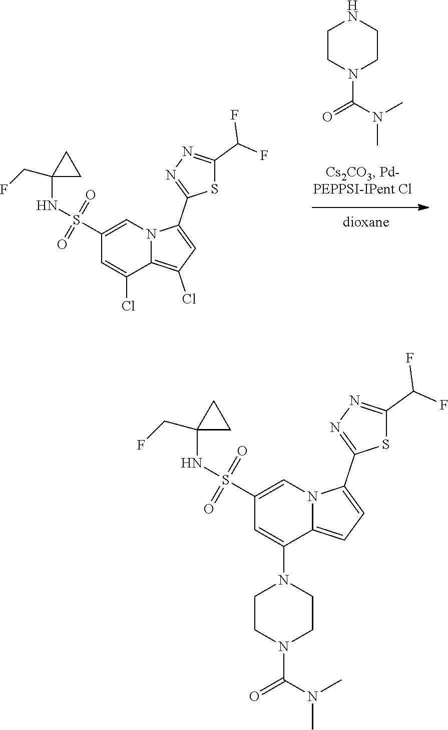 [SMILES: O=S(=O)(NC1(CF)CC1)c1cc(Cl)c2c(Cl)cc(-c3nnc(C(F)F)s3)n2c1 ; CN(C)C(=O)N1CCNCC1 ; CN(C)C(=O)N1CCN(c2cc(S(=O)(=O)NC3(CF)CC3)cn3c(-c4nnc(C(F)F)s4)ccc23)CC1]

A mixture of 1,8-dichloro-3-(5-(difluoromethyl)-1,3,4-thiadiazol-2-yl)-N-(1-(fluoromethyl)cyclopropyl)indolizine-6-sulfonamide (25 mg, 53.04 μmol), N,N-dimethylpiperazine-1-carboxamide (16.68 mg, 106.09 μmol), Cs₂CO₃ (34.57 mg, 106.09 μmol) and Pd-PEPPSI-IPentCl o-picoline (1 mg, 1.03 μmol) in dioxane (0.5 mL) was degassed and purged with Ar (3×) in glovebox. Then, the mixture was stirred at 100° C. for 16 h under Ar atmosphere outside of the glove box. The mixture was concentrated under vacuum. The residue was diluted with MeOH (3 mL), filtered, and the filtrate was purified by preparative HPLC (column: Phenomenex Luna C18 100*30 mm*5 μm; mobile phase: A: 0.225% formic acid in water, B: MeCN; B %: 37%-67%, 8 min) and lyophilized directly to give the product 4-(3-(5-(difluoromethyl)-1,3,4-thiadiazol-2-yl)-6-(N-(1-(fluoromethyl)cyclopropyl)sulfamoyl)indolizin-8-yl)-N,N-dimethylpiperazine-1-carboxamide (0.91 mg, 1.53 μmol, 2.88% yield, 93.47% purity) as a yellow solid.

RT 0.563 min (method 3); m/z 558.0 (M+H)⁺ (ESI⁺); ¹H NMR (DMSO-d₆, 400 MHz): 9.86 (s, 1H), 8.66 (s, 1H), 7.81 (d, J=4.8 Hz, 1H), 7.79-7.51 (m, 1H), 6.93 (d, J=4.8 Hz, 1H), 6.79 (d, J=1.2 Hz, 1H), 4.39-4.08 (m, 2H), 3.37 (t, J=5.2 Hz, 4H), 3.19 (t, J=4.8 Hz, 4H), 2.80 (s, 6H), 0.81 (d, J=4.4 Hz, 2H), 0.76 (d, J=4.4 Hz, 2H)

### Preparation of Example 120

### 4-(1-chloro-3-(5-(difluoromethyl)-1,3,4-thiadiazol-2-yl)-6-(N-(1-(fluoromethyl)cyclopropyl)sulfamoyl)indolizin-8-yl)-N,N-dimethylpiperazine-1-carboxamide

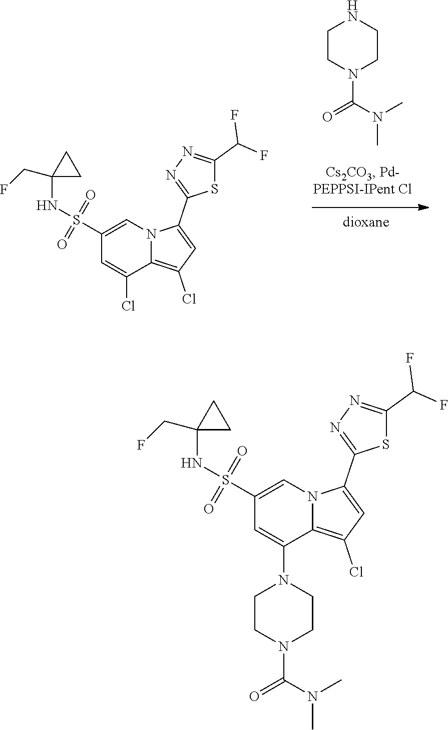 [SMILES: O=S(=O)(NC1(CF)CC1)c1cc(Cl)c2c(Cl)cc(-c3nnc(C(F)F)s3)n2c1 ; CN(C)C(=O)N1CCNCC1 ; CN(C)C(=O)N1CCN(c2cc(S(=O)(=O)NC3(CF)CC3)cn3c(-c4nnc(C(F)F)s4)cc(Cl)c23)CC1]

To a solution of 1,8-dichloro-3-(5-(difluoromethyl)-1,3,4-thiadiazol-2-yl)-N-(1-(fluoromethyl)cyclopropyl)indolizine-6-sulfonamide (20 mg, 42.44 μmol) and N,N-dimethylpiperazine-1-carboxamide (6.67 mg, 42.44 μmol) in dioxane (0.5 mL) was added Pd-PEPPSI-IPentCl o-picoline (4.13 mg, 4.24 μmol) and Cs₂CO₃ (27.65 mg, 84.87 μmol) under N₂. The mixture was stirred at 80° C. for 5 h and concentrated under vacuum. The residue was diluted with MeOH (3 mL), filtered, and the filtrate was purified by preparative TLC (SiO₂, Petroleum ether/Ethyl acetate=0:1) and lyophilized directly to give the product 4-(1-chloro-3-(5-(difluoromethyl)-1,3,4-thiadiazol-2-yl)-6-(N-(1-(fluoromethyl)cyclopropyl)sulfamoyl)indolizin-8-yl)-N,N-dimethylpiperazine-1-carboxamide (3.48 mg, 5.74 μmol, 13.52% yield, 97.595% purity) as a yellow solid.

RT 0.492 min (0.8 min method 3); m/z 592.1 (M+H)⁺ (ESI⁺); ¹H NMR (CDCl₃, 400 MHz): 10.11 (d, J=1.2 Hz, 1H), 7.33 (s, 1H), 7.20-6.93 (m, 1H), 6.82 (d, J=0.8 Hz, 1H), 5.46 (s, 1H), 4.34-4.20 (m, 2H), 3.83-2.96 (m, 8H), 2.90 (s, 6H), 1.18-1.12 (m, 2H), 0.88-0.83 (m, 2H).

### Preparation of Intermediate 121.1

### 1-bromo-8-chloro-3-(5-(difluoromethyl)-1,3,4-thiadiazol-2-yl)indolizine-6-sulfonyl bromide

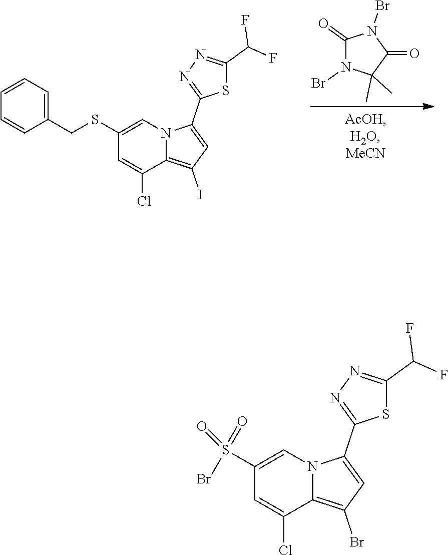 [SMILES: FC(F)c1nnc(-c2cc(I)c3c(Cl)cc(SCc4ccccc4)cn23)s1 ; CC1(C)C(=O)N(Br)C(=O)N1Br ; O=S(=O)(Br)c1cc(Cl)c2c(Br)cc(-c3nnc(C(F)F)s3)n2c1]

To a solution of 2-(6-(benzylthio)-8-chloro-1-iodoindolizin-3-yl)-5-(difluoromethyl)-1,3,4-thiadiazole (400 mg, 749.37 μmol), AcOH (90.00 mg, 1.50 mmol, 85.71 μL) and water (27.01 mg, 1.50 mmol, 27.01 μL) in ACN (4 mL) was added 1,3-dibromo-5,5-dimethylimidazolidine-2,4-dione (428.52 mg, 1.50 mmol) at 0° C. The mixture was stirred at 0° C. for 2 h, then poured into water (20 mL). The aqueous phase was extracted with DCM (10 mL, 2×). The combined organic phase was washed with brine (10 mL), dried with anhydrous Na₂SO₄, filtered and concentrated under vacuum at 20° C. The residue was purified by column chromatography (SiO₂, Petroleum ether/Ethyl acetate=I/O to 20/1) to give the product 1-bromo-8-chloro-3-(5-(difluoromethyl)-1,3,4-thiadiazol-2-yl)indolizine-6-sulfonyl bromide (260 mg, 512.26 μmol, 68.36% yield) as a brown solid.

### Preparation of Example 121

### 1-bromo-8-chloro-3-(5-(difluoromethyl)-1,3,4-thiadiazol-2-yl)-N-(1-(fluoromethyl)cyclopropyl)indolizine-6-sulfonamide

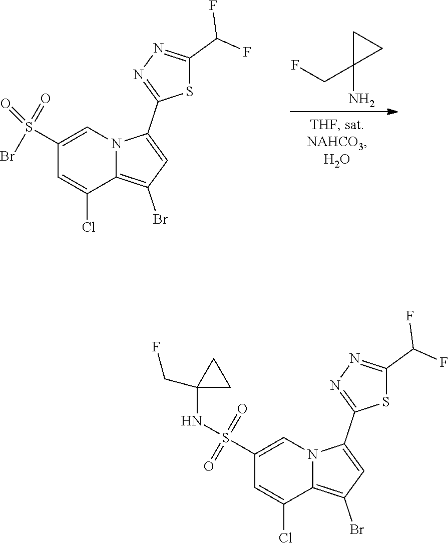 [SMILES: O=S(=O)(Br)c1cc(Cl)c2c(Br)cc(-c3nnc(C(F)F)s3)n2c1 ; NC1(CF)CC1 ; O=S(=O)(NC1(CF)CC1)c1cc(Cl)c2c(Br)cc(-c3nnc(C(F)F)s3)n2c1]

To solution of 1-(fluoromethyl)cyclopropanamine (50.21 mg, 399.87 μmol, HCl salt), NaHCO₃ (215.17 mg, 2.56 mmol, 99.62 μL), H₂O (2 mL) in THE (2 mL) was added 1-bromo-8-chloro-3-(5-(difluoromethyl)-1,3,4-thiadiazol-2-yl)indolizine-6-sulfonyl bromide (260 mg, 512.26 μmol) at 0° C. The mixture was stirred at 0° C. for 2 h and, then poured into water (20 mL). The aqueous phase was extracted with EtOAc (10 mL, 2×). The combined organic phase was washed with brine (20 mL), dried with anhydrous Na₂SO₄, filtered and concentrated under vacuum. The residue was purified by column chromatography (SiO₂, Petroleum ether/Ethyl acetate=I/O to 10/1) to give the product 1-bromo-8-chloro-3-(5-(difluoromethyl)-1,3,4-thiadiazol-2-yl)-N-(1-(fluoromethyl)cyclopropyl)indolizine-6-sulfonamide (100 mg, 193.89 μmol, 37.85% yield) as a yellow solid.

¹H NMR (DMSO-d₆, 400 MHz): 10.09 (d, J=1.2 Hz, 1H), 8.83 (s, 1H), 8.14 (s, 1H), 7.78-7.49 (m, 1H), 7.41 (d, J=1.2 Hz, 1H), 4.30 (d, J=48.4), 0.84-0.74 (m, 4H)

### Preparation of Example 122

### 8-chloro-3-(5-(difluoromethyl)-1,3,4-thiadiazol-2-yl)-N-(1-(fluoromethyl)cyclopropyl)-1-(3-methyl-3-(methylamino)but-1-yn-1-yl)indolizine-6-sulfonamide

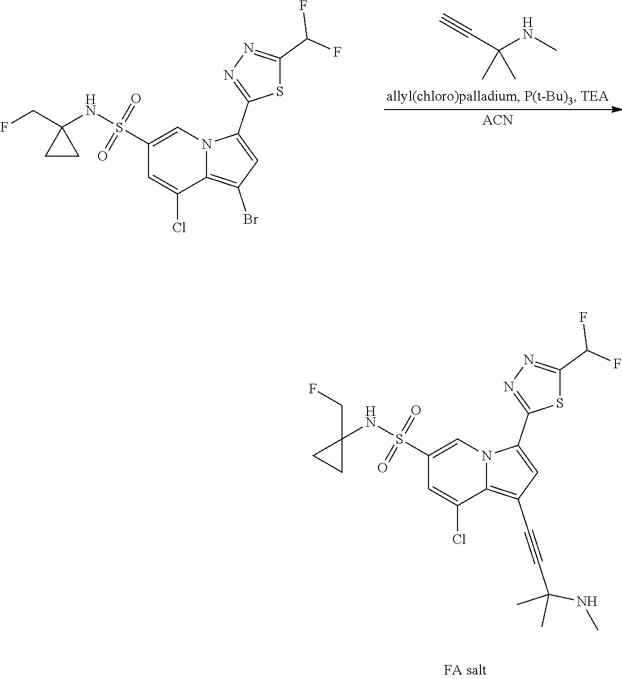

A mixture of 1-bromo-8-chloro-3-(5-(difluoromethyl)-1,3,4-thiadiazol-2-yl)-N-(1-(fluoromethyl)cyclopropyl)indolizine-6-sulfonamide (10 mg, 19.39 μmol), allyl(chloro)palladium (354.74 ug, 1.94 μmol), P(t-Bu)₃ (7.85 mg, 3.88 μmol, 9.10 μL, 10% w/w in hexane solution) and TEA (3.92 mg, 38.78 μmol, 5.40 μL) in ACN (0.2 mL) was degassed and purged with N₂ (3×). Then, N, 2-dimethylbut-3-yn-2-amine (3.77 mg, 38.78 μmol, 3.23 μL) was added to the mixture through an injection syringe. The mixture was stirred at 20° C. for 1 h. The mixture was concentrated under vacuum. The residue was purified by preparative HPLC (column: Phenomenex Luna C18 150*25 mm*10 μm; mobile phase: A: 0.225% formic acid in water, B: MeCN; B %: 18%-48%, 10 min) and lyophilized directly to give the product 8-chloro-3-(5-(difluoromethyl)-1,3,4-thiadiazol-2-yl)-N-(1-(fluoromethyl)cyclopropyl)-1-(3-methyl-3-(methylamino)but-1-yn-1-yl)indolizine-6-sulfonamide (3.06 mg, 5.29 μmol, 27.30% yield, 100% purity, FA salt) as a white solid.

RT 0.409 min (method 4); m/z 554.0 (M+Na)⁺ (ESI⁺); ¹H NMR (DMSO-d₆, 400 MHz): 10.10 (d, J=1.2 Hz, 1H), 8.86 (br s, 1H), 8.20 (s, 1H), 8.07 (s, 1H), 7.82-7.54 (m, 1H), 7.44 (d, J=1.2 Hz, 1H), 4.39-4.12 (m, 2H), 2.39 (s, 3H), 1.37 (s, 6H), 0.91-0.84 (m, 2H), 0.83-0.77 (m, 2H).

### Preparation of Intermediate 123.1

### 1-(3-((tert-butyldimethylsilyl)oxy)prop-1-yn-1-yl)-8-chloro-3-(5-(difluoromethyl)-1,3,4-thiadiazol-2-yl)-N-(1-(fluoromethyl)cyclopropyl)indolizine-6-sulfonamide

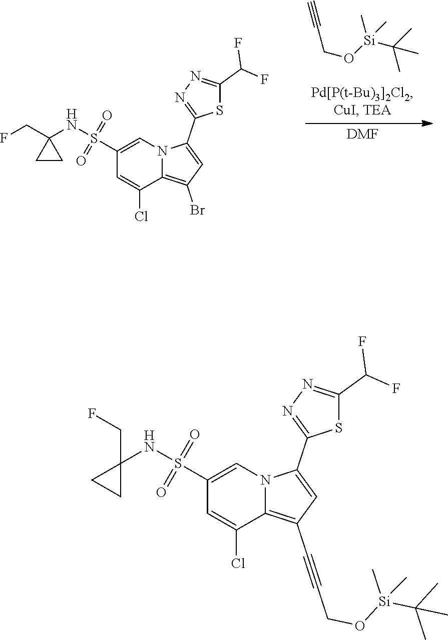

A mixture of 1-bromo-8-chloro-3-(5-(difluoromethyl)-1,3,4-thiadiazol-2-yl)-N-(1-(fluoromethyl)cyclopropyl)indolizine-6-sulfonamide (70 mg, 135.72 μmol), CuI (2.58 mg, 13.57 μmol), TEA (363.50 mg, 3.59 mmol, 0.5 mL) and Pd[P(t-Bu)₃]₂Cl₂ (10.02 mg, 13.57 μmol) in DMF (1 mL) was degassed and purged with N₂ (3×). Then, tert-butyldimethyl(prop-2-yn-1-yloxy)silane (46.23 mg, 271.45 μmol, 55.04 μL) was added to the mixture through an injection syringe. The mixture was stirred at 100° C. for 16 h under N₂ atmosphere, then, poured into water (30 mL). The aqueous phase was extracted with EtOAc (10 mL, 2×). The combined organic phase was washed with brine (20 mL), dried with anhydrous Na₂SO₄, filtered and concentrated under vacuum. The residue was purified by preparative TLC (SiO₂, Petroleum ether:Ethyl acetate=3:1) and lyophilized directly to give the product 1-(3-((tert-butyldimethylsilyl)oxy)prop-1-yn-1-yl)-8-chloro-3-(5-(difluoromethyl)-1,3,4-thiadiazol-2-yl)-N-(1-(fluoromethyl)cyclopropyl)indolizine-6-sulfonamide (10 mg, 16.52 μmol, 12.18% yield) as a yellow solid.

RT 0.593 min (method 4); m/z 605.1 (M+H)⁺ (ESI⁺).

### Preparation of Example 123

### 8-chloro-3-(5-(difluoromethyl)-1,3,4-thiadiazol-2-yl)-N-(1-(fluoromethyl)cyclopropyl)-1-(3-hydroxyprop-1-yn-1-yl)indolizine-6-sulfonamide

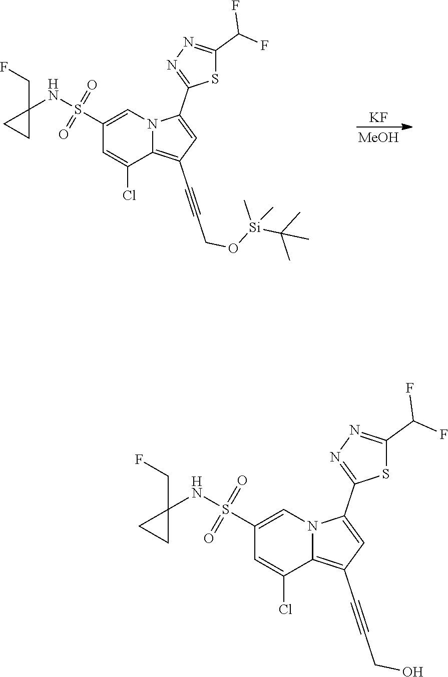 [SMILES: "MeOH" ; CC(C)(C)[Si](C)(C)OCC#Cc1cc(-c2nnc(C(F)F)s2)n2cc(S(=O)(=O)NC3(CF)CC3)cc(Cl)c12 ; "KF" ; O=S(=O)(NC1(CF)CC1)c1cc(Cl)c2c(C#CCO)cc(-c3nnc(C(F)F)s3)n2c1]

To a solution of 1-(3-((tert-butyldimethylsilyl)oxy)prop-1-yn-1-yl)-8-chloro-3-(5-(difluoromethyl)-1,3,4-thiadiazol-2-yl)-N-(1-(fluoromethyl)cyclopropyl)indolizine-6-sulfonamide (10 mg, 16.52 μmol) in MeOH (0.2 mL) was added KF (1.92 mg, 33.05 μmol). The mixture was stirred at 20° C. for 4 h and, then concentrated under vacuum. The residue was diluted with MeOH (3 mL), filtered, and the filtrate was purified by preparative HPLC (column: Waters Xbridge 150*25 mm*10 μm; mobile phase: A: 10 mM aqueous solution of NH₄HCO₃, B: MeCN; B %: 25%-55%, 8 min) and lyophilized directly to give the product 8-chloro-3-(5-(difluoromethyl)-1,3,4-thiadiazol-2-yl)-N-(1-(fluoromethyl)cyclopropyl)-1-(3-hydroxyprop-1-yn-1-yl)indolizine-6-sulfonamide (1.27 mg, 2.59 μmol, 15.66% yield, 100% purity) as a yellow solid.

RT 0.438 min (method 4); m/z 513.0 (M+Na)⁺ (ESI⁺); ¹H NMR (DMSO-d₆, 400 MHz): 10.09 (d, J=1.2 Hz, 1H), 8.85 (br, 1H), 8.11 (s, 1H), 7.67 (t, J=53.2 Hz, 1H), 7.46 (d, J=1.2 Hz, 1H), 5.33 (t, J=6.0 Hz, 1H), 4.36 (d, J=6.0 Hz, 2H), 4.31-4.18 (d, J=48.4 Hz, 2H), 0.89-0.86 (m, 2H), 0.81-0.78 (m, 2H).

### Preparation of Intermediate 124.1

### tert-butyl 3-((8-chloro-3-(5-(difluoromethyl)-1,3,4-thiadiazol-2-yl)-6-(N-(1-(fluoromethyl)cyclopropyl)sulfamoyl)indolizin-1-yl)methylene)azetidine-1-carboxylate

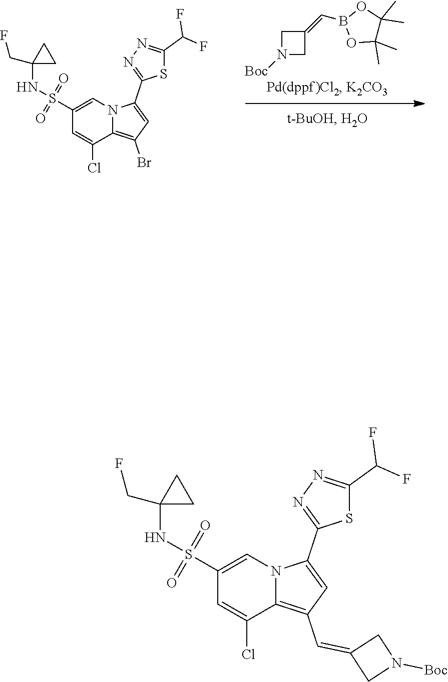 [SMILES: O=S(=O)(NC1(CF)CC1)c1cc(Cl)c2c(Br)cc(-c3nnc(C(F)F)s3)n2c1 ; CC(C)(C)OC(=O)N1CC(=CB2OC(C)(C)C(C)(C)O2)C1 ; CC(C)(C)OC(=O)N1CC(=Cc2cc(-c3nnc(C(F)F)s3)n3cc(S(=O)(=O)NC4(CF)CC4)cc(Cl)c23)C1]

A solution of 1-bromo-8-chloro-3-(5-(difluoromethyl)-1,3,4-thiadiazol-2-yl)-N-(1-(fluoromethyl)cyclopropyl)indolizine-6-sulfonamide (60 mg, 116.33 μmol), tert-butyl 3-((4,4,5,5-tetramethyl-1,3,2-dioxaborolan-2-yl)methylene)azetidine-1-carboxylate (41.21 mg, 139.60 μmol) and Pd(dppf)Cl₂ (8.47 mg, 11.63 μmol), K₃PO₄ (1.5 M in water, 155.11 μL) in t-BuOH (1 mL) was degassed, purged with N₂ (3×), and stirred at 60° C. for 4 h under N₂ atmosphere. To this mixture was further added tert-butyl 3-((4,4,5,5-tetramethyl-1,3,2-dioxaborolan-2-yl)methylene)azetidine-1-carboxylate (41.21 mg, 139.60 μmol), K₃PO₄ (1.5 M in water, 100 μL) and Pd(dppf)Cl₂ (8.47 mg, 11.63 μmol) and the reaction was stirred at 60° C. for additional 4 h. Again, to the mixture was added tert-butyl 3-((4,4,5,5-tetramethyl-1,3,2-dioxaborolan-2-yl)methylene)azetidine-1-carboxylate (41.21 mg, 139.60 μmol) and Pd(dppf)Cl₂ (8.47 mg, 11.63 μmol) and the reaction was stirred at 60° C. for another 4 h. The mixture was poured into water (10 mL) and the aqueous layer was extracted with EtOAc (5 mL, 2×). The combined organic phase was washed with brine (10 mL), dried with anhydrous Na₂SO₄, filtered and concentrated under vacuum. The residue was purified by preparative TLC (SiO₂, Petroleum ether:Ethyl acetate=3:2) to give the product tert-butyl 3-((8-chloro-3-(5-(difluoromethyl)-1,3,4-thiadiazol-2-yl)-6-(N-(1-(fluoromethyl)cyclopropyl)sulfamoyl)indolizin-1-yl)methylene)azetidine-1-carboxylate (17 mg, 28.14 μmol, 24.19% yield) as a yellow solid

RT 0.538 min (method 4); m/z 503.9 (M-Boc+H)⁺ (ESI).

### Preparation of Intermediate 124.2

### tert-butyl 3-((8-chloro-3-(5-(difluoromethyl)-1,3,4-thiadiazol-2-yl)-6-(N-(1-(fluoromethyl)cyclopropyl)sulfamoyl)indolizin-1-yl)methyl)azetidine-1-carboxylate

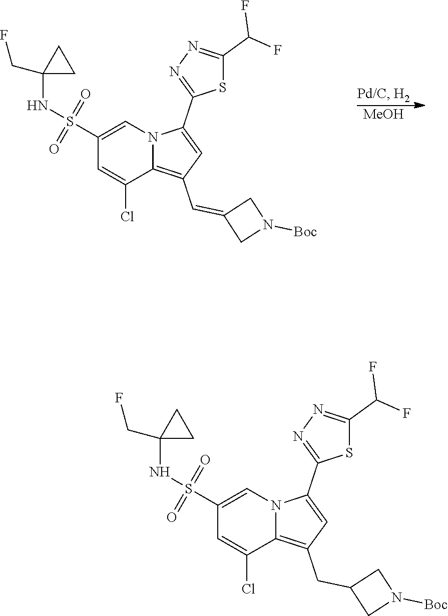 [SMILES: "MeOH" ; CC(C)(C)OC(=O)N1CC(=Cc2cc(-c3nnc(C(F)F)s3)n3cc(S(=O)(=O)NC4(CF)CC4)cc(Cl)c23)C1 ; CC(C)(C)OC(=O)N1CC(Cc2cc(-c3nnc(C(F)F)s3)n3cc(S(=O)(=O)NC4(CF)CC4)cc(Cl)c23)C1]

To a solution of tert-butyl 3-((8-chloro-3-(5-(difluoromethyl)-1,3,4-thiadiazol-2-yl)-6-(N-(1-(fluoromethyl)cyclopropyl)sulfamoyl)indolizin-1-yl)methylene)azetidine-1-carboxylate (20 mg, 33.11 μmol) in MeOH (1 mL) was added Pd/C (10 mg, 10% purity) under N₂ atmosphere. The suspension was degassed and purged with H₂ (3×) and then stirred at 20° C. for 2 h. The mixture was filtered and the filtrate was concentrated under vacuum to give the product tert-butyl 3-((8-chloro-3-(5-(difluoromethyl)-1,3,4-thiadiazol-2-yl)-6-(N-(1-(fluoromethyl)cyclopropyl)sulfamoyl)indolizin-1-yl)methyl)azetidine-1-carboxylate (5 mg, 8.25 μmol, 24.92% yield) as a yellow solid.

RT 0.529 min (method 4); m/z 506.2 (M-BocH)⁺ (ESI⁺).

### Preparation of Example 124

### 1-(azetidin-3-ylmethyl)-8-chloro-3-(5-(difluoromethyl)-1,3,4-thiadiazol-2-yl)-N-(1-(fluoromethyl)cyclopropyl)indolizine-6-sulfonamide

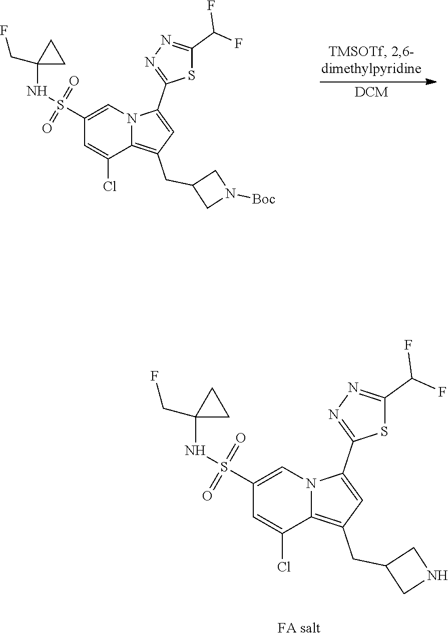 [SMILES: CC(C)(C)OC(=O)N1CC(Cc2cc(-c3nnc(C(F)F)s3)n3cc(S(=O)(=O)NC4(CF)CC4)cc(Cl)c23)C1 ; O=S(=O)(NC1(CF)CC1)c1cc(Cl)c2c(CC3CNC3)cc(-c3nnc(C(F)F)s3)n2c1]

To a solution of tert-butyl 3-((8-chloro-3-(5-(difluoromethyl)-1,3,4-thiadiazol-2-yl)-6-(N-(1-(fluoromethyl)cyclopropyl)sulfamoyl)indolizin-1-yl)methyl)azetidine-1-carboxylate (15 mg, 24.75 μmol) and 2,6-dimethylpyridine (21.22 mg, 197.99 μmol, 23.06 μL) in DCM (0.5 mL) was added TMSOTf (33.00 mg, 148.50 μmol, 26.83 μL) drop-wise at 0° C. The mixture was stirred at 20° C. for 1.5 h and then concentrated under vacuum. The residue was purified by preparative HPLC (column: Phenomenex luna C18 150*25 mm*10 μm; mobile phase: A: 0.225% formic acid in water, B: MeCN; B %: 13%-43%, 8 min) and lyophilized directly to give the product 1-(azetidin-3-ylmethyl)-8-chloro-3-(5-(difluoromethyl)-1,3,4-thiadiazol-2-yl)-N-(1-(fluoromethyl)cyclopropyl)indolizine-6-sulfonamide (0.6 mg, 1.14 μmol, 4.61% yield, 96.24% purity, FA salt) as a yellow solid.

RT 0.366 min (method 4); m/z 506.0 (M+H)⁺ (ESI⁺); ¹H NMR (DMSO-d₆, 400 MHz): 10.08 (d, J=1.2 Hz, 1H), 8.40 (s, 1H), 7.86-7.78 (m, 1H), 7.75 (s, 1H), 7.34 (d, J=1.2 Hz, 1H), 4.35-4.15 (m, 2H), 3.85-3.81 (m, 2H), 3.61-3.57 (m, 2H), 3.13-3.05 (m, 2H), 2.60 (s, 1H), 0.88-0.82 (m, 2H), 0.82-0.76 (m, 2H).

### Preparation of Example 125

### 8-chloro-3-(5-(difluoromethyl)-1,3,4-thiadiazol-2-yl)-N-(1-(fluoromethyl)cyclopropyl)-1-isobutylindolizine-6-sulfonamide

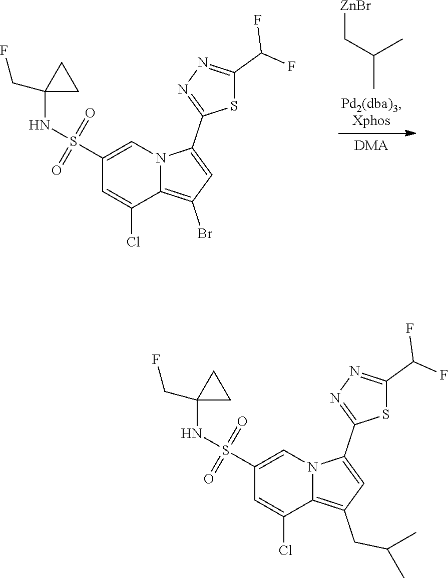

To a mixture of 1-bromo-8-chloro-3-(5-(difluoromethyl)-1,3,4-thiadiazol-2-yl)-N-(1-(fluoromethyl)cyclopropyl)indolizine-6-sulfonamide (20 mg, 38.78 μmol), Pd₂(dba)₃ (3.55 mg, 3.88 μmol) and XPhos (3.70 mg, 7.76 μmol) in DMA (1 mL) was added isobutylzinc(II) bromide (0.5 M, 310.22 μL) under N₂. Then, the mixture was stirred at 80° C. for 1 h under N₂ atmosphere. The mixture was filtered and the filtrate was concentrated under reduce pressure. The residue was purified by reversed-phase flash (ISCO®; 20 g Flash Column Welch Ultimate XB_C18 20-40 μm; 120 A, mobile phase: A: 0.1% formic acid in water, B: MeCN; B %: 5%-95%@40 mL/min). The resulting impure product was then purified by preparative HPLC (column: Phenomenex luna C18 150*25 mm*10 μm; mobile phase: A: 0.225% formic acid in water, B: MeCN; B %: 65%-95%, 58 min) and lyophilized directly to give another impure product which was further purified by preparative TLC (SiO₂, Petroleum ether:Ethyl acetate=3:1) to give the product 8-chloro-3-(5-(difluoromethyl)-1,3,4-thiadiazol-2-yl)-N-(1-(fluoromethyl)cyclopropyl)-1-isobutylindolizine-6-sulfonamide (1.58 mg, 3.04 μmol, 7.84% yield, 94.8% purity) as a white solid.

RT 0.596 min (method 4); m/z 492.9 (M+H)⁺ (ESI⁺); ¹H NMR (DMSO-d₆, 400 MHz): 10.10 (s, 1H), 8.80 (s, 1H), 7.81 (s, 1H), 7.67 (t, J=53.2 Hz, 1H), 7.32 (s, 1H), 4.26 (t, J=48.8 Hz, 1H), 2.93 (d, J=6.8 Hz, 2H), 1.95-1.99 (m, 1H), 0.96 (d, J=6.4 Hz, 6H), 0.87 (t, J=4.8 Hz, 2H), 0.81 (t, J=4.0 Hz, 2H).

### Preparation of Example 126

### 3-(5-(difluoromethyl)-1,3,4-thiadiazol-2-yl)-N-(1-(fluoromethyl)cyclopropyl)indolizine-6-sulfonamide

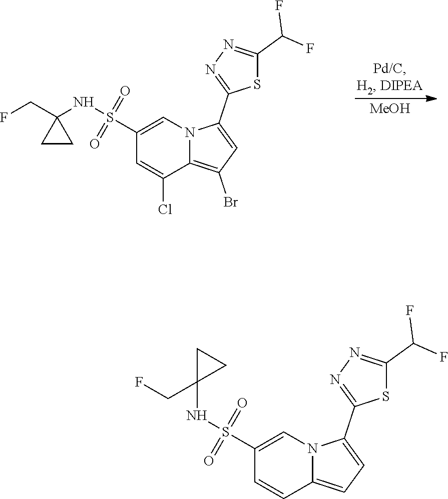 [SMILES: "MeOH" ; O=S(=O)(NC1(CF)CC1)c1cc(Cl)c2c(Br)cc(-c3nnc(C(F)F)s3)n2c1 ; O=S(=O)(NC1(CF)CC1)c1ccc2ccc(-c3nnc(C(F)F)s3)n2c1]

To a solution of 1-bromo-8-chloro-3-(5-(difluoromethyl)-1,3,4-thiadiazol-2-yl)-N-(1-(fluoromethyl)cyclopropyl)indolizine-6-sulfonamide (20 mg, 38.78 μmol) in MeOH (0.5 mL) was added Pd/C (5 mg, 10% purity) and DIPEA (5.01 mg, 38.78 μmol, 6.75 μL) under N₂ atmosphere. The suspension was degassed and purged with H₂ for 3 times. Then, the reaction was stirred at 30° C. for 2 h, filtered and the filtrate was concentrated under vacuum. The residue was purified by preparative HPLC (column: Phenomenex luna C18 150*25 mm*10 μm; mobile phase: A: 0.225% formic acid in water, B: MeCN; B %: 40%-70%, 10 min) and lyophilized directly to give the product 3-(5-(difluoromethyl)-1,3,4-thiadiazol-2-yl)-N-(1-(fluoromethyl)cyclopropyl)indolizine-6-sulfonamide (960.00 ug, 2.37 μmol, 6.11% yield, 99.29% purity) as a white solid.

RT 0.558 min (method 3); m/z 402.9 (M+H)⁺ (ESI⁺); ¹H NMR (CDCl₃, 400 MHz): 10.39 (s, 1H), 7.65 (d, J=9.2 Hz, 1H), 7.48 (d, J=4.8 Hz, 1H), 7.34 (dd, J=9.6, 1.6 Hz, 1H), 7.20-6.93 (m, 1H), 6.77 (d, J=4.4 Hz, 1H), 5.47 (s, 1H), 4.29 (d, J=48.4 Hz, 2H), 1.16-1.10 (m, 2H), 0.88-0.83 (m, 2H)

### Preparation of Example 127

### 3-(5-(difluoromethyl)-1,3,4-thiadiazol-2-yl)-N-(1-(fluoromethyl)cyclopropyl)-1-isobutylindolizine-6-sulfonamide

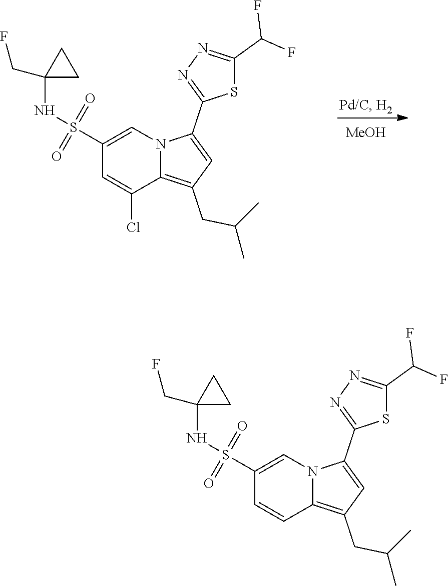 [SMILES: "MeOH" ; CC(C)Cc1cc(-c2nnc(C(F)F)s2)n2cc(S(=O)(=O)NC3(CF)CC3)cc(Cl)c12 ; CC(C)Cc1cc(-c2nnc(C(F)F)s2)n2cc(S(=O)(=O)NC3(CF)CC3)ccc12]

To a solution of 8-chloro-3-(5-(difluoromethyl)-1,3,4-thiadiazol-2-yl)-N-(1-(fluoromethyl)cyclopropyl)-1-isobutylindolizine-6-sulfonamide (10 mg, 20.29 μmol) in MeOH (1 mL) was added Pd/C (5 mg, 10% purity) and DIPEA (2.62 mg, 20.29 μmol, 3.53 μL) under N₂ atmosphere. The suspension was degassed and purged with H₂ (3×). Then, the mixture was stirred at 30° C. for 1 h filtered and the filtrate was concentrated under vacuum. The resulting crude was purified by preparative HPLC (column: Phenomenex luna C18 150*25 mm*10 μm; mobile phase: A: 0.225% formic acid in water, B: MeCN; B %: 70%-100%, 10 min) and lyophilized directly to give the product 3-(5-(difluoromethyl)-1,3,4-thiadiazol-2-yl)-N-(1-(fluoromethyl)cyclopropyl)-1-isobutylindolizine-6-sulfonamide (960.00 ug, 1.91 μmol, 9.43% yield, 91.39% purity) as a yellow solid.

RT 0.470 min (method 3); m/z 458.9 (M+H)⁺ (ESI⁺); ¹H NMR (CDCl₃, 400 MHz): 10.33 (s, 1H), 7.58 (d, J=9.2 Hz, 1H), 7.29 (s, 1H), 7.25 (d, J=9.2 Hz, 1H), 7.05 (t, J=53.2 Hz, 1H), 5.43 (s, 1H), 4.29 (d, J=48.0 Hz, 2H), 2.67 (d, J=6.8 Hz, 2H), 2.00-1.92 (m, 1H), 1.16-1.11 (m, 2H), 0.98 (d, J=6.4 Hz, 6H), 0.91-0.86 (m, 2H).

### Preparation of Intermediate 128.1

### Tert-butyl 4-(8-chloro-3-(5-(difluoromethyl)-1,3,4-thiadiazol-2-yl)-6-(N-(1-(fluoromethyl)cyclopropyl)sulfamoyl)indolizin-1-yl)-5,6-dihydropyridine-1(2H)-carboxylate

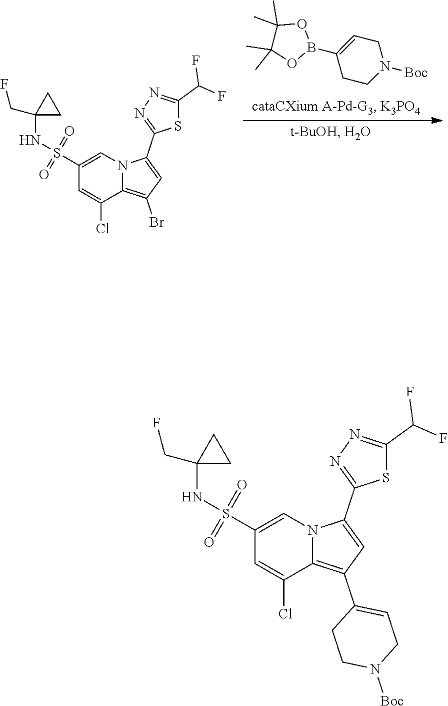 [SMILES: O=S(=O)(NC1(CF)CC1)c1cc(Cl)c2c(Br)cc(-c3nnc(C(F)F)s3)n2c1 ; CC(C)(C)OC(=O)N1CC=C(B2OC(C)(C)C(C)(C)O2)CC1 ; CC(C)(C)OC(=O)N1CC=C(c2cc(-c3nnc(C(F)F)s3)n3cc(S(=O)(=O)NC4(CF)CC4)cc(Cl)c23)CC1]

A mixture of 1-bromo-8-chloro-3-(5-(difluoromethyl)-1,3,4-thiadiazol-2-yl)-N-(1-(fluoromethyl)cyclopropyl)indolizine-6-sulfonamide (30 mg, 58.17 μmol), tert-butyl 4-(4,4,5,5-tetramethyl-1,3,2-dioxaborolan-2-yl)-5,6-dihydropyridine-1(2H)-carboxylate (25.18 mg, 81.43 μmol), Pd(dppf)Cl₂ (4.26 mg, 5.82 μmol), K₂CO₃ (16.08 mg, 116.33 μmol) and H₂O (0.025 mL) in dioxane (0.5 mL) was degassed and purged with N₂ (3×). Then, the mixture was stirred at 80° C. for 2 h under N₂ atmosphere and poured into water (10 mL). The aqueous phase was extracted with EtOAc (5 mL, 2×). The combined organic phase was washed with brine (10 mL), dried over anhydrous Na₂SO₄, filtered and concentrated under vacuum. The residue was purified by preparative TLC (SiO₂, Petroleum ether:Ethyl acetate=2:1) to give the product tert-butyl 4-(8-chloro-3-(5-(difluoromethyl)-1,3,4-thiadiazol-2-yl)-6-(N-(1-(fluoromethyl)cyclopropyl)sulfamoyl)indolizin-1-yl)-5,6-dihydropyridine-1(2H)-carboxylate (12.75 mg, 20.63 μmol, 35.46% yield) as a yellow solid.

RT 0.656 min (method 3); m/z 561.9 (M−56+H)⁺ (ESI⁺); ¹H NMR (DMSO-d₆, 400 MHz): 10.34 (d, J=1.6 Hz, 1H), 7.34 (d, J=1.2 Hz, 1H), 7.31 (s, 1H), 7.08-6.92 (m, 1H), 5.75 (br s, 1H), 5.46 (s, 1H), 4.33 (t, J=48.4 Hz, 2H), 4.11-4.08 (m, 2H), 3.68 (t, J=5.6 Hz, 2H), 2.51-2.47 (m, 2H), 1.52 (s, 9H), 1.14 (br d, J=4.0 Hz, 2H), 0.90-0.87 (m, 2H).

### Preparation of Example 128

### 8-chloro-3-(5-(difluoromethyl)-1,3,4-thiadiazol-2-yl)-N-(1-(fluoromethyl)cyclopropyl)-1-(1,2,3,6-tetrahydropyridin-4-yl)indolizine-6-sulfonamide

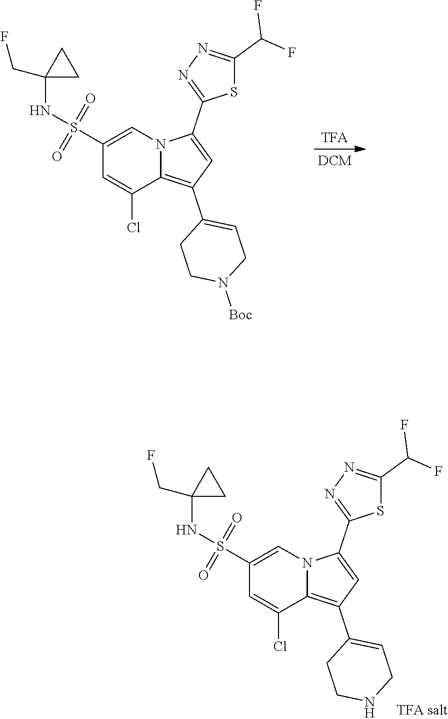 [SMILES: CC(C)(C)OC(=O)N1CC=C(c2cc(-c3nnc(C(F)F)s3)n3cc(S(=O)(=O)NC4(CF)CC4)cc(Cl)c23)CC1 ; O=S(=O)(NC1(CF)CC1)c1cc(Cl)c2c(C3=CCNCC3)cc(-c3nnc(C(F)F)s3)n2c1]

To a solution of tert-butyl 4-(8-chloro-3-(5-(difluoromethyl)-1,3,4-thiadiazol-2-yl)-6-(N-(1-(fluoromethyl)cyclopropyl)sulfamoyl)indolizin-1-yl)-5,6-dihydropyridine-1(2H)-carboxylate (10.00 mg, 16.18 μmol) in DCM (0.5 mL) was added TFA (1.5 mL). The mixture was stirred at 20° C. for 1 h and, then concentrated under vacuum. The residue was purified by preparative HPLC (column: Welch Ultimate C18 150*25 mm*5 μm; mobile phase: A: 0.225% TFA in water, B: MeCN; B %: 21%-51%, 10 min) and lyophilized directly to give the product 8-chloro-3-(5-(difluoromethyl)-1,3,4-thiadiazol-2-yl)-N-(1-(fluoromethyl)cyclopropyl)-1-(1,2,3,6-tetrahydropyridin-4-yl)indolizine-6-sulfonamide (1.6 mg, 3.09 μmol, 19.09% yield, 99.99% purity, TFA salt) as a white solid.

RT 0.390 min (method 4); m/z 518.0 (M+H)⁺ (ESI⁺); ¹H NMR (DMSO-d₆, 400 MHz): 10.13 (d, J=1.2 Hz, 1H), 8.89 (d, J=3.2 Hz, 1H), 8.84 (s, 1H), 7.82-7.80 (m, 1H), 7.80-7.52 (m, 1H), 7.43 (d, J=1.2 Hz, 1H), 5.80 (br, 1H), 4.27 (d, J=48.4 Hz, 2H), 3.79 (d, J=1.2 Hz, 2H), 3.44-3.35 (m, 2H), 2.69-2.61 (m, 2H), 0.95-0.75 (m, 4H).

### Preparation of Example 129

### 3-(5-(difluoromethyl)-1,3,4-thiadiazol-2-yl)-N-(1-methylcyclopropyl)-8-(2-oxa-7-azaspiro[3.5]nonan-7-yl)imidazo[1,5-a]pyridine-6-sulfonamide

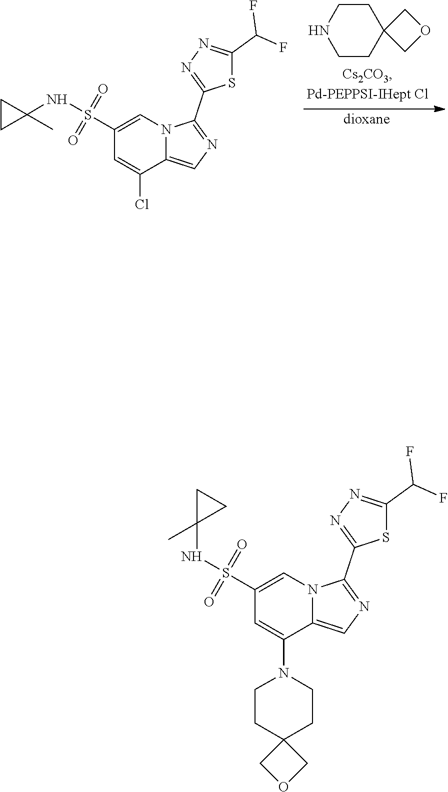

To a mixture of 8-chloro-3-(5-(difluoromethyl)-1,3,4-thiadiazol-2-yl)-N-(1-methylcyclopropyl)imidazo[1,5-a]pyridine-6-sulfonamide (40 mg, 95.27 μmol) in dioxane (1 mL) was added 2-oxa-7-azaspiro[3.5]nonane (24.23 mg, 190.54 μmol), Cs₂CO₃ (93.12 mg, 285.81 μmol) and Pd-PEPPSI-IPentCl o-picoline (8.20 mg, 9.53 μmol). The reaction mixture was degassed with N₂ (3×) and the mixture was stirred at 90° C. for 1 h. The reaction mixture was filtered and the filtrate was concentrated under vacuum. The resulting residue was purified by preparative TLC (ethyl acetate) to give an impure product which was further triturated with MeOH (1 mL) to give the product 3-(5-(difluoromethyl)-1,3,4-thiadiazol-2-yl)-N-(1-methylcyclopropyl)-8-(2-oxa-7-azaspiro[3.5]nonan-7-yl)imidazo[1,5-a]pyridine-6-sulfonamide (9.89 mg, 19.15 μmol, 20.10% yield, 98.86% purity) as a yellow solid.

RT 0.473 min (method 4); m/z 511.3 (M+H)⁺ (ESI⁺); ¹H NMR (CDCl₃, 400 MHz): 9.84 (s, 1H), 7.70 (s, 1H), 7.08 (t, J=53.6 Hz, 1H), 6.63 (d, J=1.2 Hz, 1H), 5.06 (s, 1H), 4.54 (s, 4H), 3.26-3.23 (m, 4H), 2.16-2.13 (m, 4H), 1.39 (s, 3H), 0.94-0.91 (m, 2H), 0.60 (d, J=1.6 Hz, 2H).

### Preparation of Example 130

### 4-(7-chloro-3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperazine-1-carboxamide

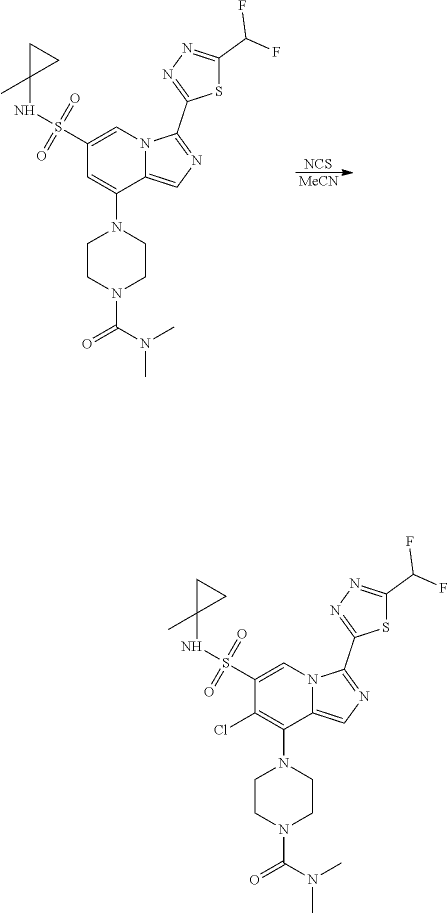 [SMILES: "MeCN" ; CN(C)C(=O)N1CCN(c2cc(S(=O)(=O)NC3(C)CC3)cn3c(-c4nnc(C(F)F)s4)ncc23)CC1 ; "NCS" ; CN(C)C(=O)N1CCN(c2c(Cl)c(S(=O)(=O)NC3(C)CC3)cn3c(-c4nnc(C(F)F)s4)ncc23)CC1]

To a mixture of 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperazine-1-carboxamide (20 mg, 37.00 μmol) in MeCN (0.5 mL) was added NCS (2.47 mg, 18.50 μmol) at 0° C. The mixture was then stirred at 25° C. for 1 h. The resulting solution was purified by preparative TLC (Ethyl acetate:Petroleum ether=1:0) and concentrated under vacuum to give the product 4-(7-chloro-3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperazine-1-carboxamide (3.30 mg, 5.62 μmol, 15.19% yield, 98% purity) as a yellow solid.

RT 0.583 min (method 4); m/z 574.9 (M+H)⁺ (ESI⁺); ¹H NMR (CDCl₃, 400 MHz): 10.20 (s, 1H), 7.85 (s, 1H), 7.09 (t, J=53.2 Hz, 1H), 5.45 (s, 1H), 3.47-3.45 (m, 4H), 3.46-3.44 (m, 4H), 2.92 (s, 6H), 1.34 (s, 3H), 0.99-0.97 (m, 2H), 0.66-0.53 (m, 2H).

### Preparation of Example 131

### 4-(1,7-dichloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide

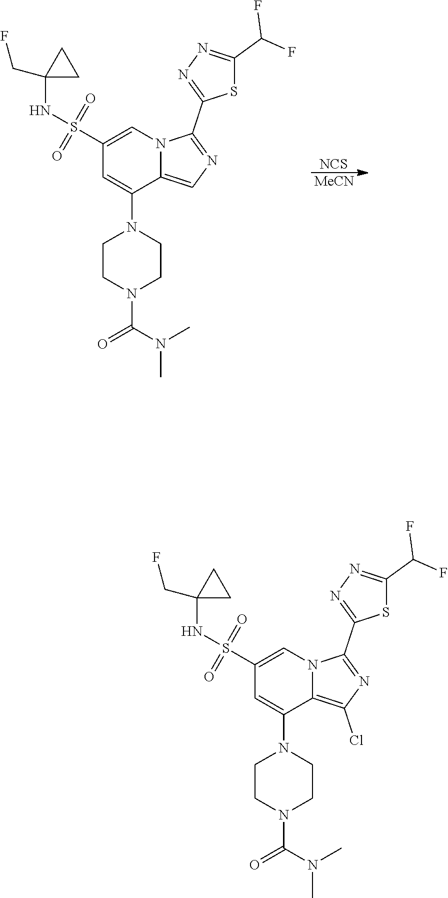 [SMILES: "MeCN" ; CN(C)C(=O)N1CCN(c2cc(S(=O)(=O)NC3(CF)CC3)cn3c(-c4nnc(C(F)F)s4)ncc23)CC1 ; "NCS" ; CN(C)C(=O)N1CCN(c2cc(S(=O)(=O)NC3(CF)CC3)cn3c(-c4nnc(C(F)F)s4)nc(Cl)c23)CC1]

To a mixture of 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide (30 mg, 53.71 μmol) in MeCN (0.5 mL) was added NCS (10.76 mg, 80.56 μmol) at 0° C. The reaction mixture was then stirred for 16 h. After filtration, the filtrate was concentrated at low temperature under vacuum. The resulting residue was purified by preparative HPLC (column: Phenomenex luna C18 150*25 mm*10 μm; mobile phase: A: 0.025% formic acid in water, B: MeCN; B %: 42%-72%, 10 min) and lyophilized directly to give the product 4-(1,7-dichloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide (9.9 mg, 15.62 μmol, 29.08% yield, 99% purity) as a yellow solid.

RT 0.506 min (method 4), m/z 628.9 (M+H)⁺ (ESI⁺); ¹H NMR (CDCl₃, 400 MHz): 10.23 (s, 1H), 7.08 (t, J=53.6 Hz, 1H), 5.77 (s, 1H), 4.28-4.16 (d, J=48.8 Hz, 2H), 3.68-3.59 (m, 4H), 3.44-3.39 (m, 2H), 3.14-3.12 (m, 2H), 2.91 (s, 6H), 1.23-1.22 (m, 2H), 0.91-0.89 (m, 2H).

### Preparation of Example 132

### 4-(7-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide

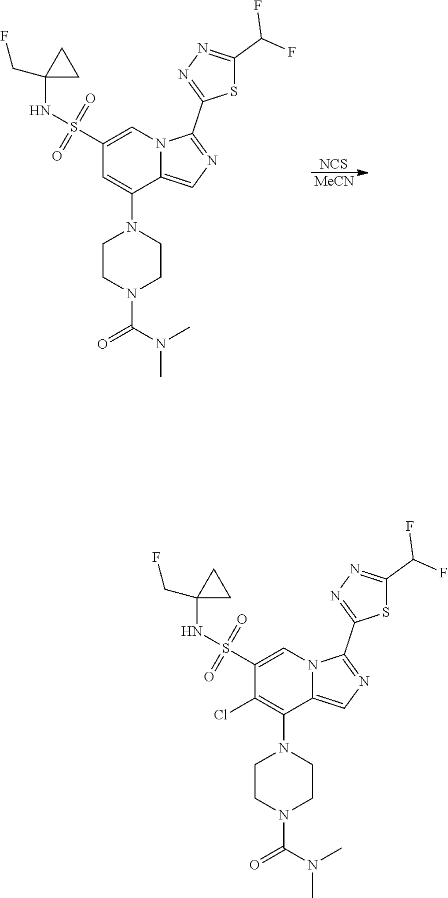 [SMILES: "MeCN" ; CN(C)C(=O)N1CCN(c2cc(S(=O)(=O)NC3(CF)CC3)cn3c(-c4nnc(C(F)F)s4)ncc23)CC1 ; "NCS" ; CN(C)C(=O)N1CCN(c2c(Cl)c(S(=O)(=O)NC3(CF)CC3)cn3c(-c4nnc(C(F)F)s4)ncc23)CC1]

To a mixture of 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide (30 mg, 53.71 μmol) in MeCN (1 mL) was added NCS (5.74 mg, 42.96 μmol) at 0° C. The reaction mixture was stirred for 16 h. After filtration, the filtrate was concentrated at low temperature under vacuum. The resulting residue was purified by preparative TLC (Ethyl acetate:Petroleum ether=1:0), concentrated under vacuum and lyophilized directly to give the product 4-(7-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N,N-dimethylpiperazine-1-carboxamide (8.03 mg, 13.27 μmol, 24.71% yield, 98% purity) as a yellow solid.

RT 0.451 min (method 4); m/z 593.1 (M+H)⁺ (ESI⁺); ¹H NMR (CDCl₃, 400 MHz): 10.18 (s, 1H), 7.85 (s, 1H), 7.08 (t, J=54.0 Hz, 1H), 5.82 (s, 1H), 4.29-4.17 (d, J=48.8 Hz, 2H), 3.48-3.46 (m, 8H), 2.92 (s, 6H), 1.22-1.20 (m, 2H), 0.90-0.86 (m, 2H).

### Preparation of Example 133.a

### tert-butyl4-(3-(5-(difluoromethyl)-1,3,4-thiadiazol-2-yl)-6-(N-(1-methylcyclopropyl)sulfamoyl)imidazo[1,5-a]pyridin-8-yl)piperidine-1-carboxylate

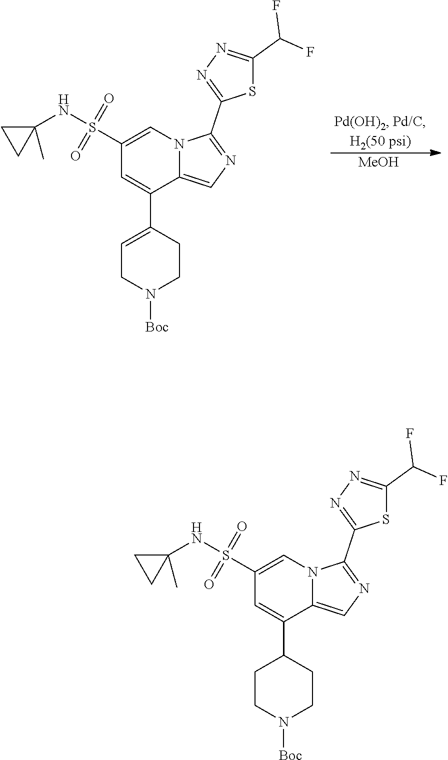 [SMILES: "MeOH" ; CC1(NS(=O)(=O)c2cc(C3=CCN(C(=O)OC(C)(C)C)CC3)c3cnc(-c4nnc(C(F)F)s4)n3c2)CC1 ; "Pd(OH)2, Pd/C,H2(50 psi)" ; CC1(NS(=O)(=O)c2cc(C3CCN(C(=O)OC(C)(C)C)CC3)c3cnc(-c4nnc(C(F)F)s4)n3c2)CC1]

To a mixture of tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-5,6-dihydropyridine-1(2H)-carboxylate (80 mg, 141.18 μmol) in MeOH (3 mL) was added Pd(OH)₂ (24 mg, 34.18 μmol, 20% purity) and Pd/C (24 mg, 141.18 μmol, 10% purity). The reaction was degassed with H₂ (3×) and then stirred at 50° C. for 16 h. The reaction mixture was filtered and the filtrate was concentrated under vacuum. The resulting residue was purified by preparative TLC (Ethyl acetate:Petroleum ether=1:1) to give the product tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)piperidine-1-carboxylate (38 mg, 58.81 μmol, 41.65% yield, 88% purity) as a yellow solid.

RT 0.573 min (method 4); m/z 591.2 (M+Na)⁺ (ESI⁺).

### Preparation of Example 133

### 3-(5-(difluoromethyl)-1,3,4-thiadiazol-2-yl)-N-(1-methylcyclopropyl)-8-(piperidin-4-yl)imidazo[1,5-a]pyridine-6-sulfonamide

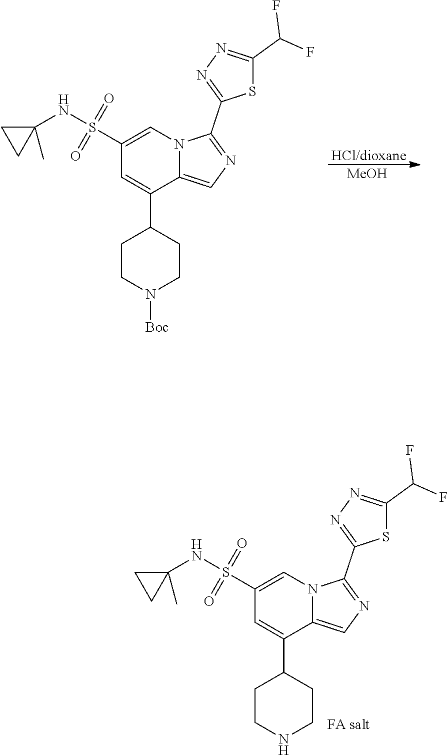 [SMILES: "MeOH" ; CC1(NS(=O)(=O)c2cc(C3CCN(C(=O)OC(C)(C)C)CC3)c3cnc(-c4nnc(C(F)F)s4)n3c2)CC1 ; "HCl/dioxane" ; CC1(NS(=O)(=O)c2cc(C3CCNCC3)c3cnc(-c4nnc(C(F)F)s4)n3c2)CC1]

A mixture of tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)piperidine-1-carboxylate (20 mg, 35.17 μmol) in HCl/dioxane (1 mL, 4N) was stirred at 25° C. for 1 h. The reaction mixture was concentrated under vacuum. The resulting residue was purified by preparative HPLC (column: Phenomenex luna C18 150*25 mm*10 μm; mobile phase: A: 0.225% formic acid in water, B: MeCN; B %: 12%-42%, 8 min) and lyophilized directly to give the product 3-(5-(difluoromethyl)-1,3,4-thiadiazol-2-yl)-N-(1-methylcyclopropyl)-8-(piperidin-4-yl)imidazo[1,5-a]pyridine-6-sulfonamide (1.07 mg, 2.04 μmol, 5.80% yield, 89.49% purity, FA salt) as a yellow solid.

RT 0.374 min (method 1); m/z 469.0 (M+H)⁺ (ESI⁺); ¹H NMR (DMSO-d₆, 400 MHz): 9.80 (s, 1H), 8.63-8.51 (m, 1H), 8.49 (s, 1H), 8.17 (s, 1H) 7.68 (t, J=53.2 Hz, 1H), 7.23 (s, 1H), 3.25-3.23 (m, 2H), 3.15-3.10 (m, 1H), 2.91-2.85 (m, 2H), 1.98-1.95 (m, 2H), 1.81-1.73 (m, 2H), 1.14 (s, 3H), 0.71 (br, 2H), 0.46 (br, 2H).

### Preparation of Example 134

### 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperidine-1-carboxamide

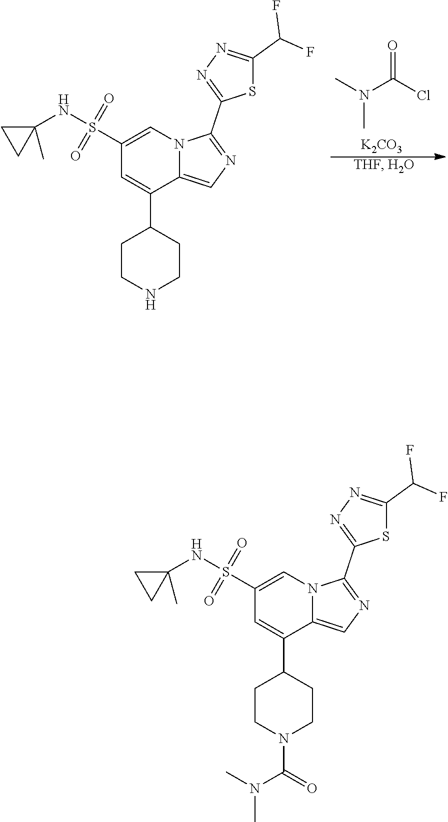

To a mixture of 3-(5-(difluoromethyl)-1,3,4-thiadiazol-2-yl)-N-(1-methylcyclopropyl)-8-(piperidin-4-yl)imidazo[1,5-a]pyridine-6-sulfonamide (10 mg, 21.34 μmol) in THE (0.5 mL) and H₂O (0.5 mL) was added K₂CO₃ (8.85 mg, 64.03 μmol) and dimethylcarbamic chloride (3.44 mg, 32.01 μmol, 2.94 μL) at 0° C. The mixture was stirred at 0° C. for 15 min, poured into water (10 mL) and extracted with EtOAc (15 mL; 2×). The organic layers were separated, mixed, dried over Na₂SO₄ and concentrated under vacuum. The resulting residue was purified by preparative HPLC (column: Phenomenex luna C18 150*25 mm*10 μm; mobile phase: A: 0.225% formic acid in water, B: MeCN; B %: 36%-66%, 8 min) and lyophilized directly to give the product 4-(3-(5-(difluoromethyl)-1,3,4-thiadiazol-2-yl)-6-(N-(1-methylcyclopropyl)sulfamoyl)imidazo[1,5-a]pyridin-8-yl)-N,N-dimethylpiperidine-1-carboxamide (1.64 mg, 2.70 μmol, 12.66% yield, 88.90% purity) as a yellow solid.

RT 0.496 min (method 4); m/z 540.0 (M+H)⁺ (ESI⁺); ¹H NMR (DMSO-d₆, 400 MHz): 9.79 (s, 1H), 8.45 (s, 1H), 8.19 (s, 1H), 7.69 (t, J=53.6 Hz, 1H), 7.26 (s, 1H), 3.72 (d, J=13.6 Hz, 2H), 3.32-3.24 (m, 1H), 2.95 (t, J=12.4 Hz, 2H), 2.78 (s, 6H), 1.94 (d, J=12.4 Hz, 2H), 1.72-1.68 (m, 2H), 1.11 (s, 3H), 0.72-0.69 (m, 2H), 0.46-0.44 (m, 2H).

### Preparation of Example 135.1

### 1-bromo-8-chloro-3-(5-(difluoromethyl)-1,3,4-thiadiazol-2-yl)imidazo[1,5-a]pyridine-6-sulfonyl chloride

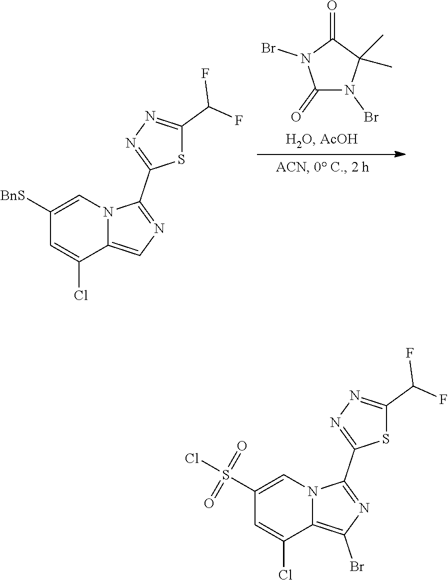

To a mixture of 2-(6-benzylsulfanyl-8-chloro-imidazo[1,5-a]pyridin-3-yl)-5-(difluoromethyl)-1,3,4-thiadiazole (1 g, 2.45 mmol) in ACN (10 mL) at 0° C. was added AcOH (293.73 mg, 4.89 mmol, 279.75 μL), water (88.14 mg, 4.89 mmol, 88.14 μL) and 1,3-dibromo-5,5-dimethyl-imidazolidine-2,4-dione (2.80 g, 9.78 mmol). Then, the reaction was stirred at 0° C. for 2 h. The resulting mixture was concentrated under vacuum (at 20° C.). The residue was diluted with DCM (30 mL), washed with ice water (20 mL; 4×), separated, dried over Na₂SO₄, and concentrated under vacuum (at 20° C.). The crude product (1.6 g) was purified by flash silica gel chromatography (ISCO®; 20 g SepaFlash® Silica Flash Column, Eluent of 0˜15% Ethyl acetate/Petroleum@50 mL/min) to give the product 1-bromo-8-chloro-3-(5-(difluoromethyl)-1,3,4-thiadiazol-2-yl)imidazo[1,5-a]pyridine-6-sulfonyl chloride (800 mg, 1.10 mmol, 44.90% yield, 69.8% purity) as a light yellow solid.

¹H NMR (CDCl₃, 400 MHz): 10.32 (d, J=1.2 Hz, 1H), 7.47 (d, J=1.2 Hz, 1H), 7.11 (t, J=53.6 Hz, 1H).

### Preparation of Example 135.2

### 1-bromo-8-chloro-3-(5-(difluoromethyl)-1,3,4-thiadiazol-2-yl)-N-(1-(fluoromethyl)cyclopropyl)imidazo[1,5-a]pyridine-6-sulfonamide

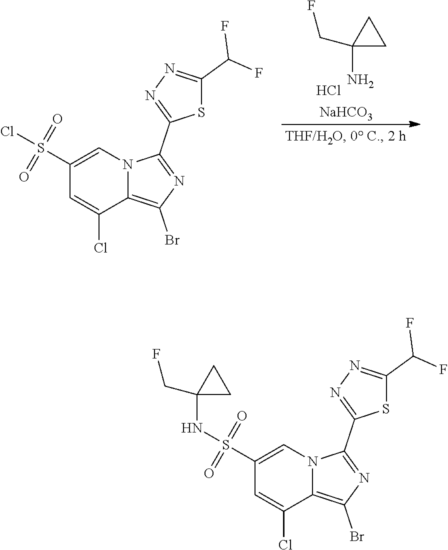

To a mixture of 1-(fluoromethyl)cyclopropanamine (217.29 mg, 1.73 mmol, HCl) in THE (8 mL) and H₂O (8 mL) was added NaHCO₃ (660.79 mg, 7.87 mmol, 305.92 μL) at 0° C. Then, 1-bromo-8-chloro-3-[5-(difluoromethyl)-1,3,4-thiadiazol-2-yl]imidazo[1,5-a]pyridine-6-sulfonyl bromide (800 mg, 1.57 mmol) in THE (2 mL) was added dropwise over 10 min and stirred at 0° C. for 2 h. The resulting mixture was quenched with saturated brine (30 mL), extracted with EtOAc (30 mL; 3×). The combined organic layer was separated, dried over anhydrous Na₂SO₄ and concentrated under vacuum. The residue was purified by flash silica gel chromatography (ISCO®; 20 g SepaFlash® Silica Flash Column, Eluent of 0˜20% Ethyl acetate/Petroleum@30 mL/min) to give the product 1-bromo-8-chloro-3-(5-(difluoromethyl)-1,3,4-thiadiazol-2-yl)-N-(1-(fluoromethyl)cyclopropyl)imidazo[1,5-a]pyridine-6-sulfonamide (200 mg, 348.34 μmol, 22.14% yield, 90% purity) as a light yellow solid.

RT 0.497 min (method 1); m/z 517.8 (M+H)⁺ (ESI⁺); ¹H NMR (CDCl₃, 400 MHz): 10.16 (s, 1H), 7.38 (s, 1H), 7.09 (t, J=53.2 Hz, 1H), 5.59 (s, 1H), 4.31 (d, J=48.4 Hz, 2H), 1.19-1.10 (m, 2H), 0.95-0.88 (m, 2H).

### Preparation of Example 135

### 8-chloro-3-(5-(difluoromethyl)-1,3,4-thiadiazol-2-yl)-N-(1-(fluoromethyl)cyclopropyl)-1-(3-methoxyprop-1-yn-1-yl)imidazo[1,5-a]pyridine-6-sulfonamide

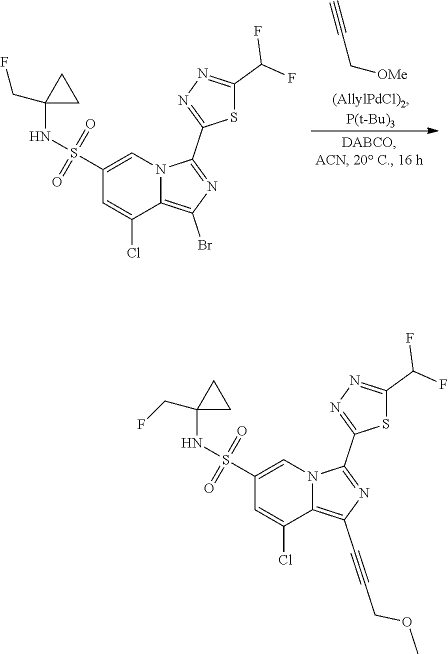

A mixture of 1-bromo-8-chloro-3-(5-(difluoromethyl)-1,3,4-thiadiazol-2-yl)-N-(1-(fluoromethyl)cyclopropyl)imidazo[1,5-a]pyridine-6-sulfonamide (100 mg, 193.52 μmol), 3-methoxyprop-1-yne (27.13 mg, 387.04 μmol, 31.91 μL), allyl(chloro)palladium (7.08 mg, 19.35 μmol), tritert-butylphosphane (78.30 mg, 38.70 μmol, 90.84 μL, 10% purity) and DABCO (43.42 mg, 387.04 μmol, 42.56 μL) in ACN (4 mL) was degassed and purged with N₂ (3×). Then, the mixture was stirred at 25° C. for 1 h, filtered and the filtrate was concentrated under vacuum. The residue was purified by flash silica gel chromatography (ISCO®; 12 g SepaFlash® Silica Flash Column, Eluent of 0˜55% Ethyl acetate/Petroleum ether @20 mL/min) to give an impure product which was triturated with MeOH (3 mL) at 20° C. for 5 min. After filtration, the filtrate was concentrated directly to give the product 8-chloro-3-(5-(difluoromethyl)-1,3,4-thiadiazol-2-yl)-N-(1-(fluoromethyl)cyclopropyl)-1-(3-methoxyprop-1-yn-1-yl)imidazo[1,5-a]pyridine-6-sulfonamide (115 mg, 225.04 μmol, 23.26% yield, 99% purity) as a yellow solid.

RT 0.485 min (method 1); m/z 506.2 (M+H)⁺ (ESI⁺); ¹H NMR (CDCl₃, 400 MHz): 10.15 (d, J=1.2 Hz, 1H), 7.41 (d, J=1.2 Hz, 1H), 7.09 (t, J=56.0 Hz, 1H), 5.56 (s, 1H), 4.45 (s, 2H), 4.31 (d, J=48.0 Hz, 2H), 3.52 (s, 3H), 1.17-1.12 (m, 2H), 0.93-0.90 (m, 2H).

### Preparation of Example 136

### 8-chloro-3-(5-(difluoromethyl)-1,3,4-thiadiazol-2-yl)-N-(1-(fluoromethyl)cyclopropyl)-1-(3-methoxypropyl)imidazo[1,5-a]pyridine-6-sulfonamide and Example 137 3-(5-(difluoromethyl)-1,3,4-thiadiazol-2-yl)-N-(1-(fluoromethyl)cyclopropyl)-1-(3-methoxypropyl)imidazo[1,5-a]pyridine-6-sulfonamide

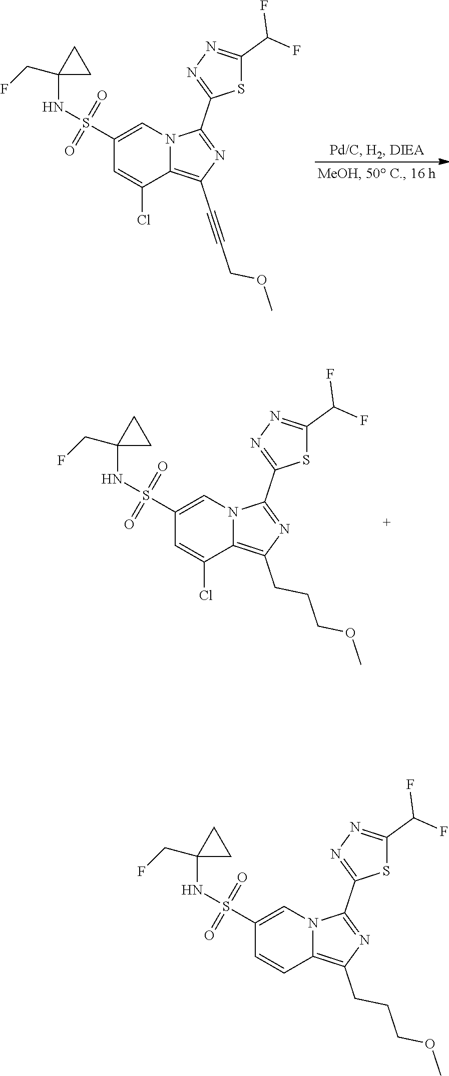 [SMILES: COCC#Cc1nc(-c2nnc(C(F)F)s2)n2cc(S(=O)(=O)NC3(CF)CC3)cc(Cl)c12 ; "Pd/C, H2, DIEA" ; COCCCc1nc(-c2nnc(C(F)F)s2)n2cc(S(=O)(=O)NC3(CF)CC3)cc(Cl)c12 ; COCCCc1nc(-c2nnc(C(F)F)s2)n2cc(S(=O)(=O)NC3(CF)CC3)ccc12]

To a solution of 8-chloro-3-[5-(difluoromethyl)-1,3,4-thiadiazol-2-yl]-N-[1-(fluoromethyl)cyclopropyl]-1-(3-methoxyprop-1-ynyl)imidazo[1,5-a]pyridine-6-sulfonamide (20 mg, 39.53 μmol) in MeOH (0.5 mL) was added Pd/C (5 mg, 10% purity) and DIEA (5.11 mg, 39.53 μmol, 6.89 μL). The reaction was degassed with H₂ (3×) and the mixture was stirred under H₂ (15 psi, 1 atm) at 50° C. for 16 h. The mixture was filtered and the filtrate was concentrated under vacuum. The residue was purified by preparative HPLC (column: Phenomenex luna C18 150*25 mm*10 μm; mobile phase: A: 0.225% formic acid in water, B: MeCN; B %: 42%-72%, 10 min) and lyophilized to give the product 8-chloro-3-(5-(difluoromethyl)-1,3,4-thiadiazol-2-yl)-N-(1-(fluoromethyl)cyclopropyl)-1-(3-methoxypropyl)imidazo[1,5-a]pyridine-6-sulfonamide (3.98 mg, 7.49 μmol, 18.95% yield, 96% purity) as a yellow solid and 8-chloro-3-(5-(difluoromethyl)-1,3,4-thiadiazol-2-yl)-N-(1-(fluoromethyl)cyclopropyl)-1-(3-methoxypropyl)imidazo[1,5-a]pyridine-6-sulfonamide (4.28 mg, 8.55 μmol, 21.63% yield, 95% purity) as a yellow solid.

### 8-chloro-3-(5-(difluoromethyl)-1,3,4-thiadiazol-2-yl)-N-(1-(fluoromethyl)cyclopropyl)-1-(3-methoxypropyl)imidazo[1,5-a]pyridine-6-sulfonamide

RT 0.522 min (method 1); m/z 510.1 (M+H)⁺ (ESI⁺); ¹H NMR (CDCl₃, 400 MHz): 10.08 (d, J=1.2 Hz, 1H), 7.25 (d, J=0.8 Hz, 1H), 7.08 (t, J=56.0 Hz, 1H), 5.50 (s, 1H), 4.32 (d, J=48.0 Hz, 1H), 3.52 (t, J=8.0 Hz, 2H), 3.38 (s, 3H), 3.31 (t, J=8.0 Hz, 2H), 2.15-2.04 (m, 2H), 1.16-1.12 (m, 2H), 0.92-0.88 (m, 2H).

### 8-chloro-3-(5-(difluoromethyl)-1,3,4-thiadiazol-2-yl)-N-(1-(fluoromethyl)cyclopropyl)-1-(3-methoxypropyl)imidazo[1,5-a]pyridine-6-sulfonamide

RT 0.492 min (method 1); m/z 475.8 (M+H)⁺ (ESI⁺); ¹H NMR (CDCl₃, 400 MHz): 10.11 (s, 1H), 7.72 (d, J=8.0 Hz, 1H), 7.25 (dd, J=1.2, 8.0 Hz, 1H), 7.08 (t, J=56.0 Hz, 1H), 5.50 (s, 1H), 4.29 (d, J=48.0 Hz, 2H), 3.43 (t, J=8.0 Hz, 2H), 3.36 (s, 3H), 3.05 (t, J=8.0 Hz, 2H), 2.12-2.05 (m, 2H), 1.15-1.11 (m, 2H), 0.89-0.86 (m, 2H).

### Preparation of Intermediate 138.1

### 2-(8-(benzyloxy)-6-(benzylthio)imidazo[1,5-a]pyridin-3-yl)-5-(difluoromethyl)-1,3,4-thiadiazole

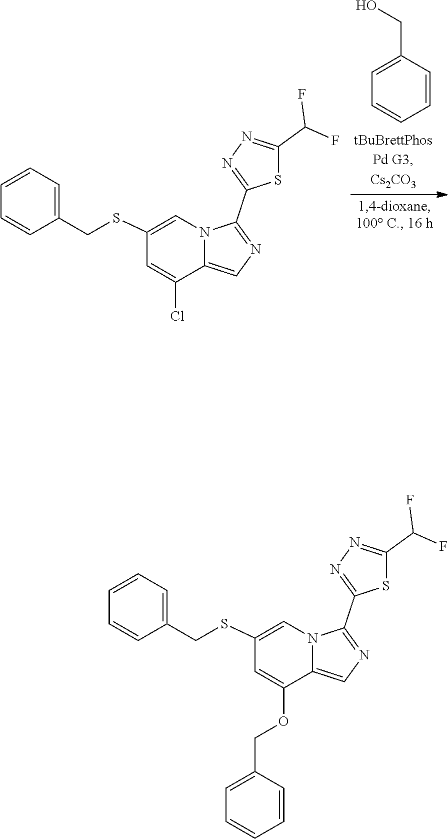 [SMILES: FC(F)c1nnc(-c2ncc3c(Cl)cc(SCc4ccccc4)cn23)s1 ; OCc1ccccc1 ; FC(F)c1nnc(-c2ncc3c(OCc4ccccc4)cc(SCc4ccccc4)cn23)s1]

To a solution of 2-(6-(benzylthio)-8-chloroimidazo[1,5-a]pyridin-3-yl)-5-(difluoromethyl)-1,3,4-thiadiazole (2 g, 4.89 mmol) in dioxane (30 mL) were added phenylmethanol (2.12 g, 19.57 mmol, 2.03 mL), Cs₂CO₃ (3.19 g, 9.78 mmol) and t-BuBrettPhos Pd G3 (417.94 mg, 489.15 μmol). The reaction mixture was stirred under N₂ at 100° C. for 16 h. After filtration, the filtrate was concentrated under reduced pressure. The residue was purified by flash silica gel chromatography (ISCO®; 40 g SepaFlash® Silica Flash Column, Eluent of 0˜20% Ethyl acetate/Petroleum ether @40 mL/min) to give the product 2-(8-(benzyloxy)-6-(benzylthio)imidazo[1,5-a]pyridin-3-yl)-5-(difluoromethyl)-1,3,4-thiadiazole (700 mg, 1.44 mmol, 29.40% yield, 98.731% purity) as a yellow solid.

RT 0.754 min (method 1), m/z 480.8 (M+H)⁺ (ESI⁺), ¹H NMR (CDCl₃, 400 MHz): 9.22 (s, 1H), 7.77 (s, 1H), 7.47-7.44 (m, 4H), 7.43-7.38 (m, 1H), 7.34-7.29 (m, 5H), 7.07 (t, J=52.0 Hz, 1H), 6.24 (s, 1H), 5.12 (s, 2H), 4.13 (s, 2H).

### Preparation of Intermediate 138.2

### 6-(benzylthio)-3-(5-(difluoromethyl)-1,3,4-thiadiazol-2-yl)imidazo[1,5-a]pyridin-8-ol

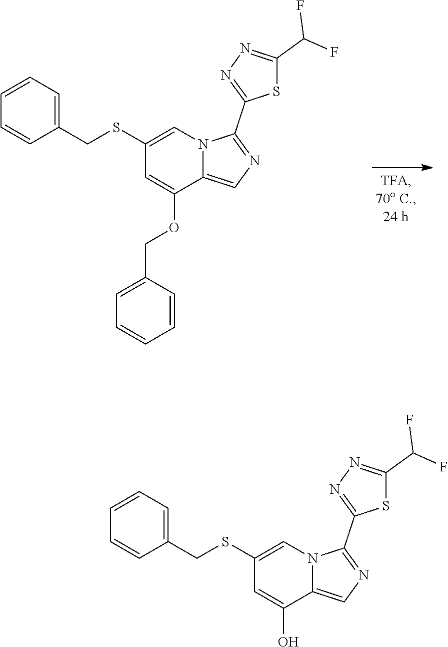 [SMILES: FC(F)c1nnc(-c2ncc3c(OCc4ccccc4)cc(SCc4ccccc4)cn23)s1 ; Oc1cc(SCc2ccccc2)cn2c(-c3nnc(C(F)F)s3)ncc12]

A solution of 2-(8-(benzyloxy)-6-(benzylthio)imidazo[1,5-a]pyridin-3-yl)-5-(difluoromethyl)-1,3,4-thiadiazole (300 mg, 624.28 μmol) in TFA (3 mL) was stirred at 70° C. for 24 h. The reaction mixture was concentrated undervacuum. The residue was dissolved in EtOAc (25 mL), washed with brine. The organic phase was separated, dried over Na₂SO₄ and concentrated under vacuum. The residue was purified by preparative TLC (Petroleum ether:Ethyl acetate=2/1) to give the product 6-(benzylthio)-3-(5-(difluoromethyl)-1,3,4-thiadiazol-2-yl)imidazo[1,5-a]pyridin-8-ol (150 mg, 291.99 μmol, 46.77% yield, 76% purity) as a yellow solid.

RT 0.471 min (method 1); m/z 390.8 (M+H)⁺ (ESI⁺); ¹H NMR (CDCl₃, 400 MHz): 9.22 (s, 1H), 7.78 (s, 1H), 7.37-7.35 (m, 2H), 7.30 (m, 1H), 7.23-7.21 (m, 2H), 7.07 (t, J=52.0 Hz, 1H), 6.31 (s, 1H), 4.17 (s, 2H).

### Preparation of Intermediate 138.3

### 2-(6-(benzylthio)-8-(3-methoxypropoxy)imidazo[1,5-a]pyridin-3-yl)-5-(difluoromethyl)-1,3,4-thiadiazole

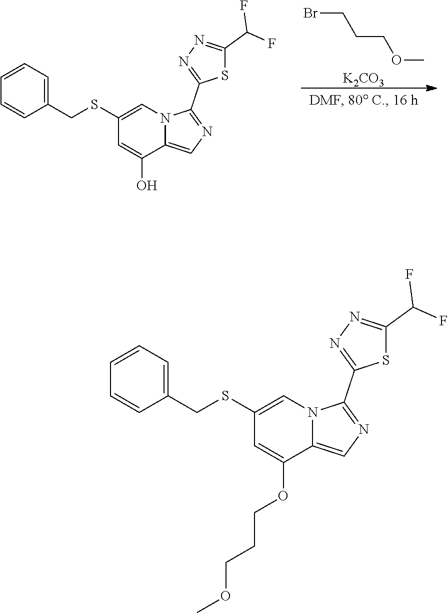

To a mixture of 6-(benzylthio)-3-(5-(difluoromethyl)-1,3,4-thiadiazol-2-yl)imidazo[1,5-a]pyridin-8-ol (40 mg, 102.45 μmol) in DMF (1 mL) was added K₂CO₃ (28.32 mg, 204.90 μmol) and 1-bromo-3-methoxypropane (31.35 mg, 204.90 μmol). The reaction was heated to 80° C. and stirred for 16 h. The reaction mixture was diluted with 20 mL H₂O, extracted with EtOAc (10 mL, 3×). The organic layers were separated, combined, washed with brine, dried over Na₂SO₄ and concentrated under vacuum. The residue was purified by preparative TLC (Petroleum ether:Ethyl acetate=2/1) to give the product 2-(6-(benzylthio)-8-(3-methoxypropoxy)imidazo[1,5-a]pyridin-3-yl)-5-(difluoromethyl)-1,3,4-thiadiazole (40 mg, 68.32 μmol, 53.35% yield, 79% purity) as a yellow solid.

RT 0.537 min (method 1); m/z 463.0 (M+H)⁺ (ESI⁺); ¹H NMR (DMSO-d₆, 400 MHz): 8.96 (s, 1H), 7.80 (s, 1H), 7.65 (t, J=52.0 Hz, 1H), 7.40-7.38 (m, 1H), 7.30-7.25 (m, 2H), 7.23-7.19 (m, 2H), 6.70 (s, 1H), 4.33 (s, 2H), 4.26 (t, J=8.0 Hz, 2H), 3.53 (t, J=8.0 Hz, 2H), 3.28 (s, 3H), 2.06-2.01 (m, 2H).

### Preparation of Intermediate 138.4

### 3-(5-(difluoromethyl)-1,3,4-thiadiazol-2-yl)-8-(3-methoxypropoxy)imidazo[1,5-a]pyridine-6-sulfonyl chloride and 1-chloro-3-(5-(difluoromethyl)-1,3,4-thiadiazol-2-yl)-8-(3-methoxypropoxy)imidazo[1,5-a]pyridine-6-sulfonyl chloride

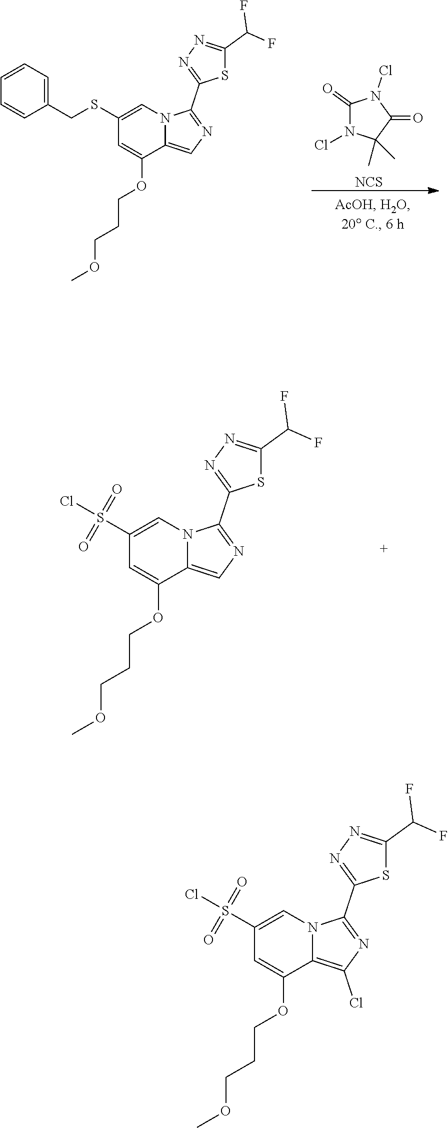

To a mixture of 2-(6-(benzylthio)-8-(3-methoxypropoxy)imidazo[1,5-a]pyridin-3-yl)-5-(difluoromethyl)-1,3,4-thiadiazole (20 mg, 43.24 μmol) in AcOH (0.4 mL) and H₂O (0.2 mL) at 0° C. was added NCS (23.10 mg, 172.96 μmol) and 1,3-dichloro-5,5-dimethylimidazolidine-2,4-dione (25.56 mg, 129.72 μmol). The mixture was stirred at 20° C. for 6 h, then diluted with DCM (15 mL), washed with brine (10 mL; 4×), dried over Na₂SO₄ and concentrated under vacuum to give the crude mixture product 3-(5-(difluoromethyl)-1,3,4-thiadiazol-2-yl)-8-(3-methoxypropoxy)imidazo[1,5-a]pyridine-6-sulfonyl chloride and 1-chloro-3-(5-(difluoromethyl)-1,3,4-thiadiazol-2-yl)-8-(3-methoxypropoxy)imidazo[1,5-a]pyridine-6-sulfonyl chloride (17 mg, crude) as a yellow solid. The crude mixture product was directly used in the next step without further purification.

### Preparation of Example 138

### 3-(5-(difluoromethyl)-1,3,4-thiadiazol-2-yl)-8-(3-methoxypropoxy)-N-(1-methylcyclopropyl)imidazo[1,5-a]pyridine-6-sulfonamide and Example 139 1-chloro-3-(5-(difluoromethyl)-1,3,4-thiadiazol-2-yl)-8-(3-methoxypropoxy)-N-(1-methylcyclopropyl)imidazo[1,5-a]pyridine-6-sulfonamide

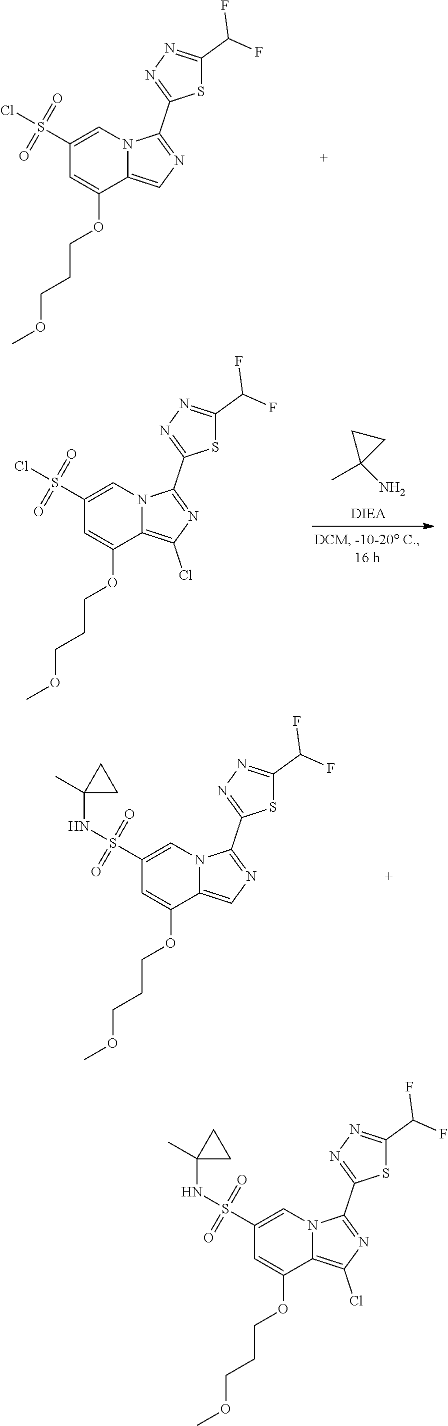

To a mixture of 1-methylcyclopropanamine (5.88 mg, 54.69 μmol, HCl) in DCM (0.5 mL) at −10° C. were added DIEA (10.60 mg, 82.03 μmol, 14.29 μL), 3-(5-(difluoromethyl)-1,3,4-thiadiazol-2-yl)-8-(3-methoxypropoxy)imidazo[1,5-a]pyridine-6-sulfonyl chloride and 1-chloro-3-(5-(difluoromethyl)-1,3,4-thiadiazol-2-yl)-8-(3-methoxypropoxy)imidazo[1,5-a]pyridine-6-sulfonyl chloride (17 mg, crude). The mixture was stirred at 20° C. for 16 h. The reaction mixture was concentrated under vacuum. The residue was diluted with MeOH (3 mL), filtered, and the filtrate was purified by preparative HPLC (column: Phenomenex luna C18 150*25 mm*10 μm; mobile phase: A: 0.225% formic acid in water, B: MeCN; B %: 45%-75%, 10 min) and lyophilized to give the product 3-(5-(difluoromethyl)-1,3,4-thiadiazol-2-yl)-8-(3-methoxypropoxy)-N-(1-methylcyclopropyl)imidazo[1,5-a]pyridine-6-sulfonamide (4.81 mg, 9.95 μmol, 36.41% yield, 98% purity) as a light yellow solid and 1-chloro-3-(5-(difluoromethyl)-1,3,4-thiadiazol-2-yl)-8-(3-methoxypropoxy)-N-(1-methylcyclopropyl)imidazo[1,5-a]pyridine-6-sulfonamide (2.0 mg, crude) as a yellow solid.

### 3-(5-(difluoromethyl)-1,3,4-thiadiazol-2-yl)-8-(3-methoxypropoxy)-N-(1-methylcyclopropyl)imidazo[1,5-a]pyridine-6-sulfonamide

RT 0.536 min (method 1); m/z 474.1 (M+H)⁺ (ESI⁺); ¹H NMR (DMSO-d₆, 400 MHz): 9.55 (s, 1H), 8.44 (s, 1H), 7.94 (s, 1H), 7.69 (t, J=52.0 Hz, 1H), 6.80 (s, 1H), 4.32 (t, J=8.0 Hz, 2H), 3.56 (t, J=8.0 Hz, 2H), 3.28 (s, 3H), 2.11 (t, J=8.0 Hz, 2H), 1.17 (s, 3H), 0.75-0.73 (m, 2H), 0.48-0.45 (m, 2H).

### 1-chloro-3-(5-(difluoromethyl)-1,3,4-thiadiazol-2-yl)-8-(3-methoxypropoxy)-N-(1-methylcyclopropyl)imidazo[1,5-a]pyridine-6-sulfonamide

RT 0.496 min (method 1); m/z 508.0 (M+H)⁺ (ESI⁺).

### Preparation of Intermediate 140.1

### 2-(6-(benzylthio)-8-((tetrahydro-2H-pyran-4-yl)oxy)imidazo[1,5-a]pyridin-3-yl)-5-(difluoromethyl)-1,3,4-thiadiazole

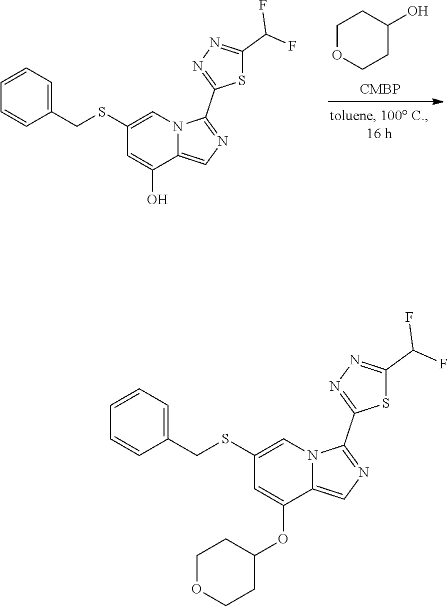 [SMILES: Oc1cc(SCc2ccccc2)cn2c(-c3nnc(C(F)F)s3)ncc12 ; OC1CCOCC1 ; FC(F)c1nnc(-c2ncc3c(OC4CCOCC4)cc(SCc4ccccc4)cn23)s1]

To a solution of 6-(benzylthio)-3-(5-(difluoromethyl)-1,3,4-thiadiazol-2-yl)imidazo[1,5-a]pyridin-8-ol (60 mg, 153.68 μmol) in toluene (2 mL) was added tetrahydro-2H-pyran-4-ol (31.39 mg, 307.35 μmol, 30.77 μL) and CMBP (74.18 mg, 307.35 μmol). The mixture was degassed and purged with N₂ (3×). The mixture was stirred at 100° C. for 16 h and then, diluted with EtOAc (45 mL). The organic layer was washed with brine (20 mL; 2×), dried over Na₂SO₄ and concentrated under vacuum. The crude residue was purified by preparative TLC (Petroleum ether:Ethyl acetate=3/1) to give the product 2-(6-(benzylthio)-8-((tetrahydro-2H-pyran-4-yl)oxy)imidazo[1,5-a]pyridin-3-yl)-5-(difluoromethyl)-1,3,4-thiadiazole (40 mg, 65.75 μmol, 42.78% yield, 78% purity) as a yellow solid.

RT 0.578 min (method 1); m/z 475.2 (M+H)⁺ (ESI⁺); ¹H NMR (CDCl₃, 400 MHz): 9.25 (s, 1H), 7.73 (s, 1H), 7.38-7.36 (m, 2H), 7.33-7.29 (m, 2H), 7.27-7.23 (m, 1H), 7.07 (t, J=52.0 Hz, 1H), 6.11 (s, 1H), 4.53-4.47 (m, 1H), 4.16 (s, 2H), 4.03-3.97 (m, 2H), 3.63-3.57 (m, 2H), 2.05-1.99 (m, 2H), 1.88-1.80 (m, 2H).

### Preparation of Intermediate 140.2

### 3-(5-(difluoromethyl)-1,3,4-thiadiazol-2-yl)-8-((tetrahydro-2H-pyran-4-yl)oxy)imidazo[1,5-a]pyridine-6-sulfonyl chloride and 1-chloro-3-(5-(difluoromethyl)-1,3,4-thiadiazol-2-yl)-8-((tetrahydro-2H-pyran-4-yl)oxy)imidazo[1,5-a]pyridine-6-sulfonyl chloride

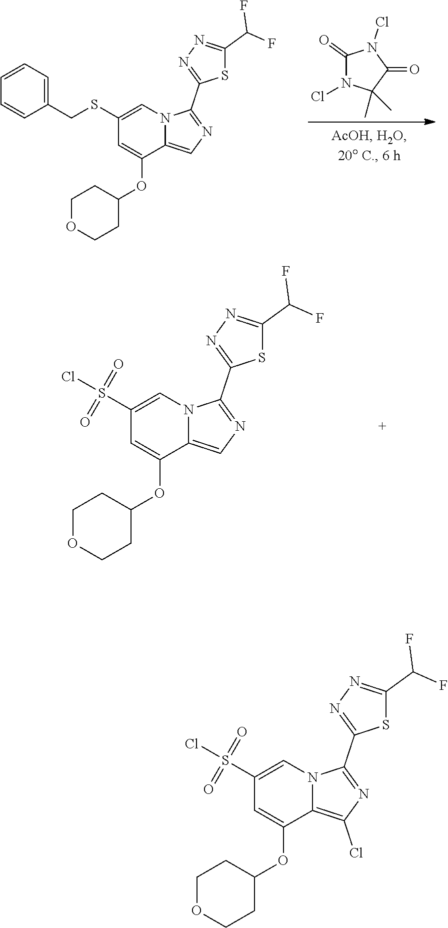 [SMILES: FC(F)c1nnc(-c2ncc3c(OC4CCOCC4)cc(SCc4ccccc4)cn23)s1 ; CC1(C)C(=O)N(Cl)C(=O)N1Cl ; O=S(=O)(Cl)c1cc(OC2CCOCC2)c2cnc(-c3nnc(C(F)F)s3)n2c1 ; O=S(=O)(Cl)c1cc(OC2CCOCC2)c2c(Cl)nc(-c3nnc(C(F)F)s3)n2c1]

To a mixture of 2-(6-benzylsulfanyl-8-tetrahydropyran-4-yloxy-imidazo[1,5-a]pyridin-3-yl)-5-(difluoromethyl)-1,3,4-thiadiazole (40 mg, 84.29 μmol) in AcOH (0.6 mL) and H₂O (0.3 mL) was added 1,3-dichloro-5,5-dimethyl-imidazolidine-2,4-dione (49.82 mg, 252.87 μmol) at 0° C. The mixture was stirred at 20° C. for 6 h and then, diluted with DCM (15 mL). The organic layer was washed with brine (20 mL; 2×), dried over Na₂SO₄, filtered and concentrated under vacuum. The crude product was used to the next step directly.

### Preparation of Example 140

### 3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide and Example 141 1-chloro-3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide

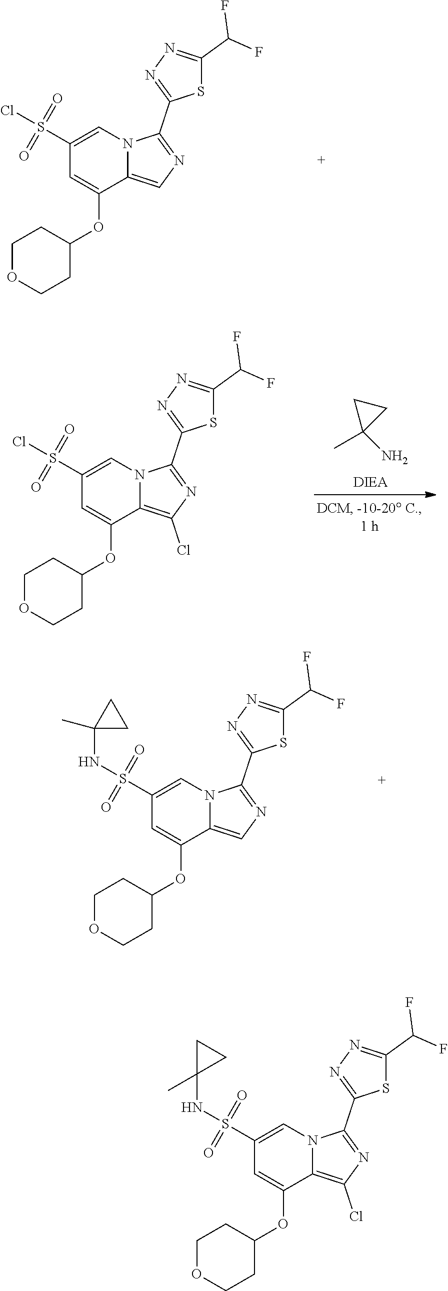

To a mixture of 1-methylcyclopropanamine (19.09 mg, 177.44 μmol, HCl salt) in DCM (0.5 mL) was added DIEA (34.40 mg, 266.15 μmol, 46.36 μL) and a mixture of 3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide (20 mg, 44.36 μmol) and 1-chloro-3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide (20 mg, 41.21 μmol) at −10° C. The mixture was stirred at 20° C. for 1 h and then concentrated under vacuum. The crude product was diluted with MeOH (3 mL), filtered, and the filtrate was purified by preparative HPLC (column: Phenomenex luna C18 150*25 mm*10 μm; mobile phase: A: 0.225% formic acid in water, B: MeCN; B %: 49%-79%, 10 min) and lyophilized to give the product 3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide (2.25 mg, 4.22 μmol, 9.51% yield, 91% purity) as a light yellow solid and the product 1-chloro-3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide (2.66 mg, 4.60 μmol, 10.38% yield, 90% purity) as a yellow solid.

### 3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide

RT 0.511 min (method 1); m/z 486.0 (M+H)⁺ (ESI⁺); ¹H NMR (CDCl₃, 400 MHz): 9.81 (s, 1H), 7.86 (s, 1H), 7.09 (t, J=52.0 Hz, 1H), 6.61 (s, 1H), 5.16 (s, 1H), 4.82-4.79 (m, 1H), 4.07-4.02 (m, 2H), 3.71-3.65 (m, 2H), 2.20-2.14 (m, 2H), 2.02-1.94 (m, 2H), 1.39 (s, 3H), 0.94 (t, J=4.0 Hz, 2H), 0.63-0.60 (m, 2H).

### 1-chloro-3-(5-(difluoromethyl)-1,3,4-thiadiazol-2-yl)-N-(1-methylcyclopropyl)-8-((tetrahydro-2H-pyran-4-yl)oxy)imidazo[1,5-a]pyridine-6-sulfonamide

RT 0.509 min (method 1); m/z 520.2 (M+H)⁺ (ESI⁺); ¹H NMR (CDCl₃, 400 MHz): 9.78 (s, 1H), 7.08 (t, J=52.0 Hz, 1H), 6.57 (s, 1H), 5.07 (s, 1H), 4.91-4.81 (m, 1H), 4.09-4.03 (m, 2H), 3.77-3.72 (m, 2H), 2.18-2.10 (m, 2H), 2.02-1.94 (m, 2H), 1.40 (s, 3H), 0.93 (t, J=4.0 Hz, 2H), 0.64-0.61 (m, 2H).

### Preparation of Intermediate 142.1

### 2-(6-(benzylthio)-8-(3-methoxypropoxy)imidazo[1,5-a]pyridin-3-yl)-5-(difluoromethyl)-1,3,4-thiadiazole

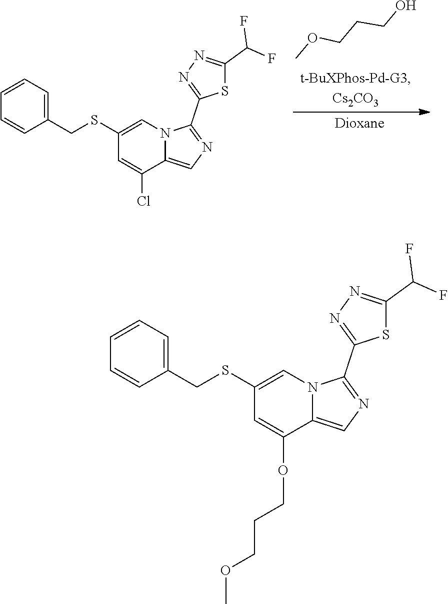

To a mixture of 2-(6-(benzylthio)-8-chloroimidazo[1,5-a]pyridin-3-yl)-5-(difluoromethyl)-1,3,4-thiadiazole (250.00 mg, 611.43 μmol) and 3-methoxypropan-1-ol (220.41 mg, 2.45 mmol, 233.98 μL) in dioxane (5 mL) was added Cs₂CO₃ (398.43 mg, 1.22 mmol) and t-BuXPhos-Pd-G3 (52.24 mg, 61.14 μmol). The mixture was degassed, purged with N₂ (3×) and stirred at 100° C. for 16 h under a N₂ atmosphere. The reaction mixture was filtered and the filtrate was concentrated under vacuum. The residue was purified by flash silica gel chromatography (ISCO®; 12 g SepaFlash® Silica Flash Column, Eluent of 0˜70% Petroleum ether/Ethyl acetate@20 mL/min) to give the product 2-(6-(benzylthio)-8-(3-methoxypropoxy)imidazo[1,5-a]pyridin-3-yl)-5-(difluoromethyl)-1,3,4-thiadiazole (120 mg, 168.64 μmol, 27.58% yield, 65% purity) as a yellow solid.

RT 0.565 min (method 1); m/z 462.9 (M+H)⁺ (ESI⁺); ¹H NMR (CDCl₃, 400 MHz): 9.20 (s, 1H), 7.72 (s, 1H), 7.38-7.36 (m, 2H), 7.32-7.29 (m, 2H), 7.26-7.22 (m, 1H), 7.07 (t, J=56.0 Hz, 1H), 6.20 (s, 1H), 4.17 (s, 2H), 4.14 (t, J=4.0 Hz, 2H), 3.59 (t, J=4.0 Hz, 2H), 3.39 (s, 3H), 2.15-2.13 (m, 2H).

### Preparation of Intermediate 142.2

### 2-(6-(benzylthio)-1-iodo-8-(3-methoxypropoxy)imidazo[1,5-a]pyridin-3-yl)-5-(difluoromethyl)-1,3,4-thiadiazole

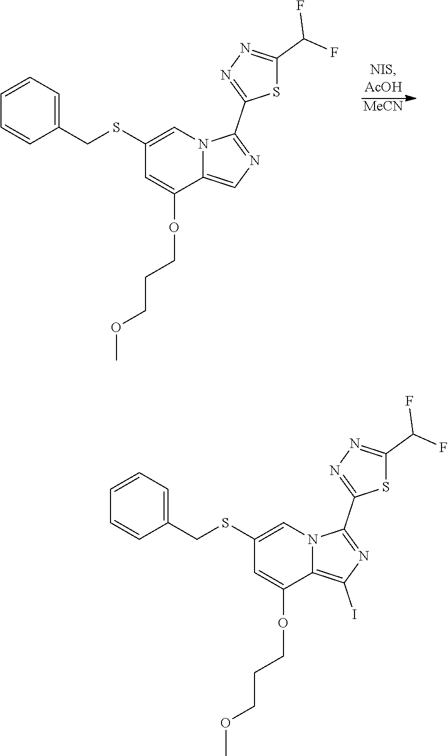 [SMILES: "MeCN" ; COCCCOc1cc(SCc2ccccc2)cn2c(-c3nnc(C(F)F)s3)ncc12 ; COCCCOc1cc(SCc2ccccc2)cn2c(-c3nnc(C(F)F)s3)nc(I)c12]

To a mixture of 2-(6-(benzylthio)-8-(3-methoxypropoxy)imidazo[1,5-a]pyridin-3-yl)-5-(difluoromethyl)-1,3,4-thiadiazole (70.00 mg, 151.34 μmol) in MeCN (1.5 mL) was added NIS (51.07 mg, 227.01 μmol) and AcOH (27.26 mg, 454.02 μmol, 25.97 μL). The reaction mixture was stirred at 25° C. for 4 h. The reaction mixture was used for next step directly.

### Preparation of Intermediate 142.3

### 3-(5-(difluoromethyl)-1,3,4-thiadiazol-2-yl)-1-iodo-8-(3-methoxypropoxy)imidazo[1,5-a]pyridine-6-sulfonyl chloride

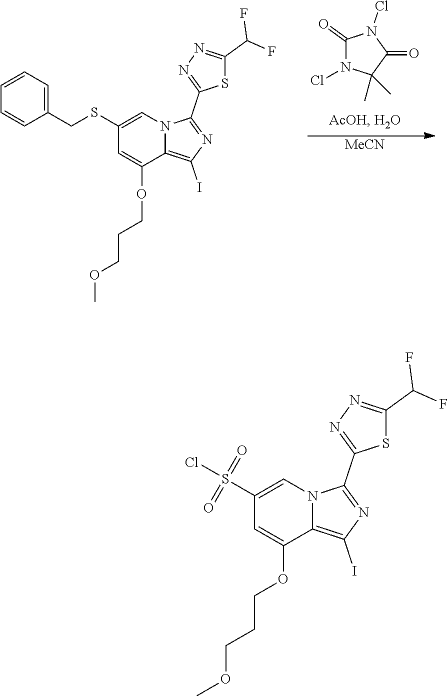 [SMILES: "MeCN" ; COCCCOc1cc(SCc2ccccc2)cn2c(-c3nnc(C(F)F)s3)nc(I)c12 ; CC1(C)C(=O)N(Cl)C(=O)N1Cl ; COCCCOc1cc(S(=O)(=O)Cl)cn2c(-c3nnc(C(F)F)s3)nc(I)c12]

To a mixture of 2-(6-(benzylthio)-1-iodo-8-(3-methoxypropoxy)imidazo[1,5-a]pyridin-3-yl)-5-(difluoromethyl)-1,3,4-thiadiazole (70 mg, 118.96 μmol, theoretical amount, reaction mixture from previous step) in MeCN (1 mL) at 0° C. was added H₂O (4.29 mg, 237.92 μmol, 4.29 μL), AcOH (14.29 mg, 237.92 μmol, 13.61 μL) and 1,3-dichloro-5,5-dimethylimidazolidine-2,4-dione (46.87 mg, 237.92 μmol). Then, the mixture was stirred at 0° C. for 2 h. The reaction mixture was used for next step directly.

### Preparation of Example 142

### N-(1-cyanocyclopropyl)-3-(5-(difluoromethyl)-1,3,4-thiadiazol-2-yl)-1-iodo-8-(3-methoxypropoxy)imidazo[1,5-a]pyridine-6-sulfonamide

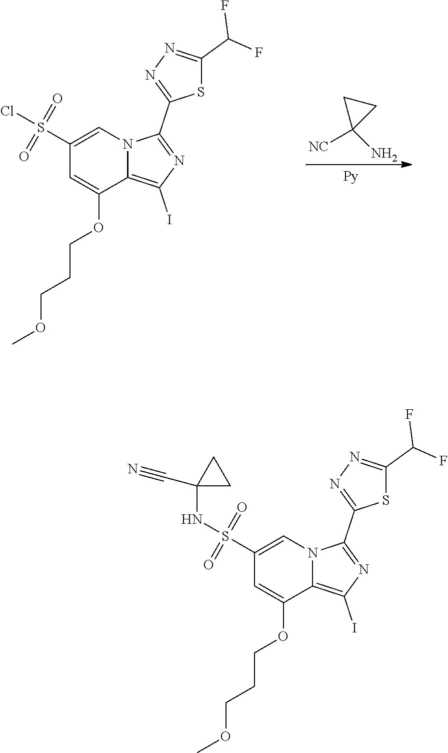 [SMILES: COCCCOc1cc(S(=O)(=O)Cl)cn2c(-c3nnc(C(F)F)s3)nc(I)c12 ; N#CC1(N)CC1 ; COCCCOc1cc(S(=O)(=O)NC2(C#N)CC2)cn2c(-c3nnc(C(F)F)s3)nc(I)c12]

To a mixture of 1-aminocyclopropanecarbonitrile (30.53 mg, 371.84 μmol) in pyridine (1.5 mL) was added 3-(5-(difluoromethyl)-1,3,4-thiadiazol-2-yl)-1-iodo-8-(3-methoxypropoxy)imidazo[1,5-a]pyridine-6-sulfonyl chloride (70 mg, 123.95 μmol, theoretical amount, reaction mixture from the previous step) at 0° C. The mixture was stirred at 0° C. for 1 h. The resulting mixture was quenched with water (30 mL) and extracted with EtOAc (30 mL, 3×). The combined organic layer was washed with brine (60 mL), separated, dried and concentrated under vacuum. The resulting residue was purified by preparative TLC (Petroleum ether:Ethyl acetate=1/1) to give the product N-(1-cyanocyclopropyl)-3-(5-(difluoromethyl)-1,3,4-thiadiazol-2-yl)-1-iodo-8-(3-methoxypropoxy)imidazo[1,5-a]pyridine-6-sulfonamide (15 mg, 22.12 μmol, 17.84% yield, 90% purity) as a yellow solid.

RT 0.491 min (method 1); m/z 611.0 (M+H)⁺ (ESI⁺).

### Preparation of Example 143

### N-(1-cyanocyclopropyl)-3-(5-(difluoromethyl)-1,3,4-thiadiazol-2-yl)-8-(3-methoxypropoxy)imidazo[1,5-a]pyridine-6-sulfonamide

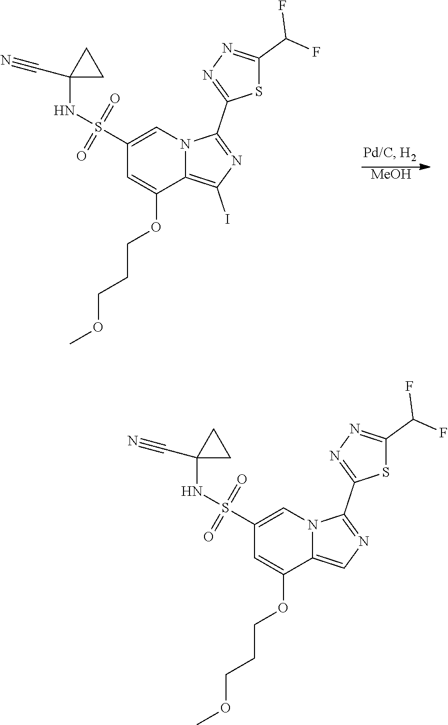 [SMILES: "MeOH" ; COCCCOc1cc(S(=O)(=O)NC2(C#N)CC2)cn2c(-c3nnc(C(F)F)s3)nc(I)c12 ; COCCCOc1cc(S(=O)(=O)NC2(C#N)CC2)cn2c(-c3nnc(C(F)F)s3)ncc12]

To a mixture of N-(1-cyanocyclopropyl)-3-(5-(difluoromethyl)-1,3,4-thiadiazol-2-yl)-1-iodo-8-(3-methoxypropoxy)imidazo[1,5-a]pyridine-6-sulfonamide (15 mg, 24.57 μmol) in MeOH (3 mL) was added Pd/C (5 mg, 10% purity). The reaction mixture was degassed with H₂ (balloon, 15 psi) (3×) and then stirred at 20° C. for 2 h. The reaction was filtered and the filtrate was concentrated under vacuum. The residue was purified by preparative HPLC (column: Phenomenex luna C18 150*25 mm*10 μm; mobile phase: A: 0.225% formic acid in water, B: MeCN; B %: 22%-52%, 10 min) to give the product N-(1-cyanocyclopropyl)-3-(5-(difluoromethyl)-1,3,4-thiadiazol-2-yl)-8-(3-methoxypropoxy)imidazo[1,5-a]pyridine-6-sulfonamide (1.9 mg, 3.92 μmol, 15.96% yield, 100% purity) as a light yellow solid.

RT 0.511 min (method 1); m/z 485.0 (M+H)⁺ (ESI⁺); ¹H NMR (400 MHz, DMSO-d₆): 9.60 (s, 1H), 7.95 (s, 1H), 7.81 (t, J=50.8 Hz, 1H), 6.76 (s, 1H), 4.34 (t, J=6.4 Hz, 2H), 3.55 (t, J=6.0 Hz, 2H), 3.28 (s, 3H), 2.12-2.09 (m, 2H), 1.40 (br, 2H), 1.31 (br, 2H).

### Preparation of Example 146a

### 8-chloro-3-(5-(difluoromethyl)-1,3,4-thiadiazol-2-yl)-1-fluoro-N-(1-(fluoromethyl)cyclopropyl)imidazo[1,5-a]pyridine-6-sulfonamide

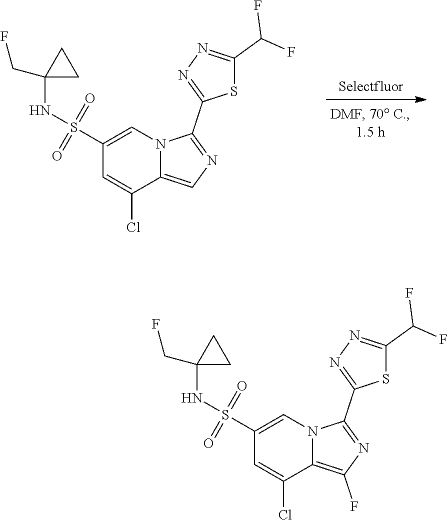 [SMILES: O=S(=O)(NC1(CF)CC1)c1cc(Cl)c2cnc(-c3nnc(C(F)F)s3)n2c1 ; O=S(=O)(NC1(CF)CC1)c1cc(Cl)c2c(F)nc(-c3nnc(C(F)F)s3)n2c1]

To a solution of 8-chloro-3-(5-(difluoromethyl)-1,3,4-thiadiazol-2-yl)-N-(1-(fluoromethyl)cyclopropyl)imidazo[1,5-a]pyridine-6-sulfonamide (10 mg, 22.84 μmol) in DMF (1 mL) was added Selectfluor (24.27 mg, 68.52 μmol). The mixture was degassed with N₂ (3×) and stirred at 70° C. for 1.5 h. The mixture was cooled to room temperature, diluted with EtOAc (10 mL) and washed with water (5 mL; 3×). The organic phase was separated, dried over Na₂SO₄, filtered and concentrated under vacuum to give a residue which was purified by preparative TLC (petroleum ether:ethyl acetate=3:1) to give the product 8-chloro-3-(5-(difluoromethyl)-1,3,4-thiadiazol-2-yl)-1-fluoro-N-(1-(fluoromethyl)cyclopropyl)imidazo[1,5-a]pyridine-6-sulfonamide (5 mg, 9.87 μmol, 28.82% yield, 90% purity) as a yellow solid

RT 0.511 min (method 1); m/z 455.9 (M+H)⁺ (ESI⁺)

### Preparation of Example 146

### 4-(3-(5-(difluoromethyl)-1,3,4-thiadiazol-2-yl)-1-fluoro-6-(N-(1-(fluoromethyl)cyclopropyl)sulfamoyl)imidazo[1,5-a]pyridin-8-yl)-N,N-dimethylpiperazine-1-carboxamide

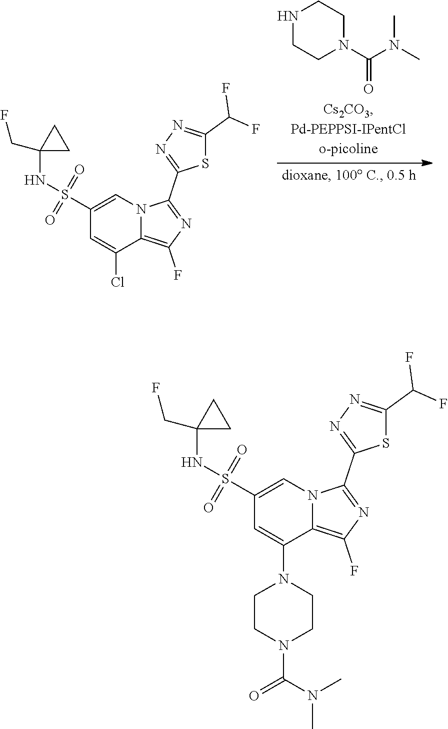 [SMILES: O=S(=O)(NC1(CF)CC1)c1cc(Cl)c2c(F)nc(-c3nnc(C(F)F)s3)n2c1 ; CN(C)C(=O)N1CCNCC1 ; CN(C)C(=O)N1CCN(c2cc(S(=O)(=O)NC3(CF)CC3)cn3c(-c4nnc(C(F)F)s4)nc(F)c23)CC1]

To a solution of 8-chloro-3-(5-(difluoromethyl)-1,3,4-thiadiazol-2-yl)-1-fluoro-N-(1-(fluoromethyl)cyclopropyl)imidazo[1,5-a]pyridine-6-sulfonamide (5 mg, 10.97 μmol), N,N-dimethylpiperazine-1-carboxamide (6.90 mg, 43.88 μmol) and Cs₂CO₃ (10.72 mg, 32.91 μmol) in dioxane (1 mL) was added Pd-PEPPSI-IPentCl o-picoline (1.07 mg, 1.10 μmol). The mixture was degassed with N₂ (3×) and stirred at 100° C. for 30 min. The mixture was cooled to 20° C., filtered and the filtrate was evaporated. The residue was purified by preparative TLC (petroleum ether:ethyl acetate=0:1) and preparative HPLC (column: Unisil 3-100 C18 Ultra 150*25 mm*10 μm; mobile phase: A: 0.225% formic acid in water, B: MeCN; B %: 38%-68%, 10 min), then lyophilized to give the product 4-(3-(5-(difluoromethyl)-1,3,4-thiadiazol-2-yl)-1-fluoro-6-(N-(1-(fluoromethyl)cyclopropyl)sulfamoyl)imidazo[1,5-a]pyridin-8-yl)-N,N-dimethylpiperazine-1-carboxamide (6 mg, 8.85 μmol, 80.63% yield, 85% purity) as a yellow solid.

RT 0.486 min (method 1); m/z 577.1 (M+H)⁺ (ESI⁺); ¹H NMR (CDCl₃ 400 MHz): 9.76 (s, 1H), 7.01 (t, J=54.0 Hz, 1H), 6.53 (s, 1H), 5.45 (s, 1H), 4.29 (d, J=48.4 Hz, 2H), 3.50 (t, J=4.4 Hz, 4H), 3.25 (t, J=4.4 Hz, 4H), 2.90 (s, 6H), 1.18-1.14 (m, 2H), 0.89 (t, J=6.4 Hz, 1H)

### Preparation of Example 147

### 8-chloro-3-(5-(difluoromethyl)-1,3,4-thiadiazol-2-yl)-N-(1-(fluoromethyl)cyclopropyl)-1-methylimidazo[1,5-a]pyridine-6-sulfonamide

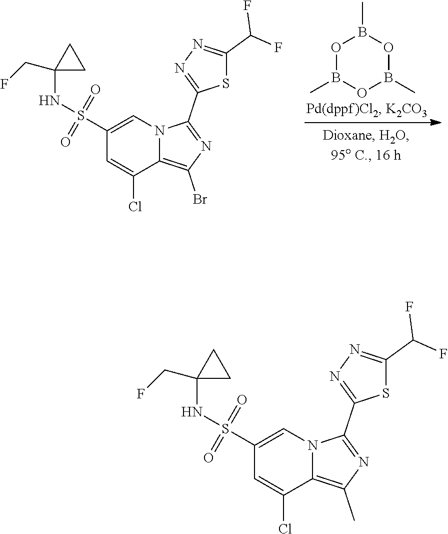

To a solution of 1-bromo-8-chloro-3-(5-(difluoromethyl)-1,3,4-thiadiazol-2-yl)-N-(1-(fluoromethyl)cyclopropyl)imidazo[1,5-a]pyridine-6-sulfonamide (20 mg, 38.70 μmol) in dioxane (1 mL) was added H₂O (0.1 mL), K₂CO₃ (10.70 mg, 77.40 μmol), 2,4,6-trimethyl-1,3,5,2,4,6-trioxatriborinane (3.5 M, 5.53 μL, 50% purity) and Pd(dppf)Cl₂ (2.83 mg, 3.87 μmol). The mixture was degassed, purged with N₂ (3×) and stirred at 80° C. for 16 h. After filtration, the filtrate was evaporated. The resulting residue was purified by preparative HPLC (column: Unisil 3-100 C18 Ultra 150*50 mm*3 μm; mobile phase: A: 0.225% formic acid in water, B: MeCN; 30%-60%, 10 min) and lyophilized directly to give the product 8-chloro-3-(5-(difluoromethyl)-1,3,4-thiadiazol-2-yl)-N-(1-(fluoromethyl)cyclopropyl)-1-methylimidazo[1,5-a]pyridine-6-sulfonamide (13 mg, 28.48 μmol, 49.06% yield, 99% purity) as a light yellow solid.

RT 0.460 min(method 1); m/z 451.9 (M+H)⁺ (ESI⁺); ¹H NMR (DMSO-d₆, 400 MHz): 9.77 (s, 1H), 8.95 (s, 1H), 7.6 (t, J=53.2 Hz, 1H), 7.33 (s, 1H), 4.25 (d, J=48.4 Hz, 2H), 2.78 (s, 3H), 0.85-0.76 (m, 4H).

### Preparation of Example 148

### 4-(3-(5-(difluoromethyl)-1,3,4-thiadiazol-2-yl)-6-(N-(1-(fluoromethyl)cyclopropyl)sulfamoyl)-1-methylimidazo[1,5-a]pyridin-8-yl)-N,N-dimethylpiperazine-1-carboxamide

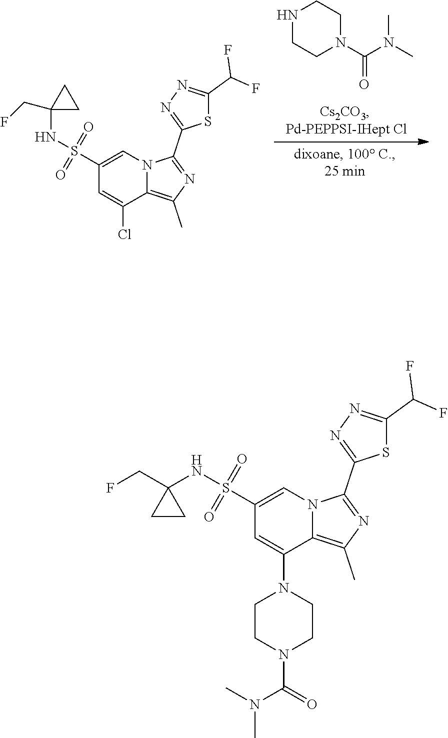 [SMILES: Cc1nc(-c2nnc(C(F)F)s2)n2cc(S(=O)(=O)NC3(CF)CC3)cc(Cl)c12 ; CN(C)C(=O)N1CCNCC1 ; Cc1nc(-c2nnc(C(F)F)s2)n2cc(S(=O)(=O)NC3(CF)CC3)cc(N3CCN(C(=O)N(C)C)CC3)c12]

To a solution of 8-chloro-3-(5-(difluoromethyl)-1,3,4-thiadiazol-2-yl)-N-(1-(fluoromethyl)cyclopropyl)-1-methylimidazo[1,5-a]pyridine-6-sulfonamide (13 mg, 28.77 μmol) in dioxane (0.5 mL) was added N,N-dimethylpiperazine-1-carboxamide (9.05 mg, 57.54 μmol), Pd-PEPPSI-IPentCl o-picoline (2.48 mg, 2.88 μmol) and Cs₂CO₃ (18.75 mg, 57.54 μmol). The reaction mixture was degassed with N₂ (3×) and stirred at 100° C. for 30 min. After filtration, the filtrate was evaporated to give a residue which was purified by preparative HPLC (column: Unisil 3-100 C18 Ultra 150*50 mm*3 μm; mobile phase: A: 0.225% formic acid in water, B: MeCN; 38%-68%, 10 min) and lyophilized directly to give the product 4-(3-(5-(difluoromethyl)-1,3,4-thiadiazol-2-yl)-6-(N-(1-(fluoromethyl)cyclopropyl)sulfamoyl)-1-methylimidazo[1,5-a]pyridin-8-yl)-N,N-dimethylpiperazine-1-carboxamide (0.5 mg, 7.68e-1 μmol, 2.67% yield, 88% purity) as a yellow solid.

RT 0.453 min (method 1); m/z 573.2 (M+H)⁺ (ESI⁺); ¹H NMR (DMSO-d₆, 400 MHz): 9.57 (s, 1H), 8.44 (s, 1H), 7.66 (t, J=53.2 Hz, 1H), 6.78 (s, 1H), 4.21 (d, J=49.2 Hz, 2H), 3.46-3.44 (m, 4H), 3.07-3.05 (m, 4H), 2.80 (s, 6H), 2.71 (s, 3H), 0.84-0.80 (m, 2H), 0.78-0.76 (m, 2H).

### Preparation of Intermediate 149.1

### (5-bromo-3-chloropyridin-2-yl)methanamine hydrochloride

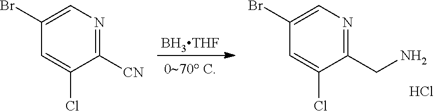 [SMILES: N#Cc1ncc(Br)cc1Cl ; NCc1ncc(Br)cc1Cl ; Cl ; "BH3·THF"]

To a mixture of 5-bromo-3-chloropicolinonitrile (2.0 g, 9.20 mmol) in tetrahydrofuran (10 mL) was added BH₃·THF (1 M, 11.04 mL) at 0° C. The mixture was stirred at 0° C. for 30 min before it was warmed to 20° C. and stirred for another 30 min at this temperature. The mixture was cooled to 0° C. and quenched with dropwise addition of methanol (10 mL) over 5 min. The mixture was heated to 70° C. and stirred for 30 min at this temperature. The reaction was concentrated under vacuum to give the crude product (2.2 g) as a light brown solid. The crude product was dissolved in HCl (aq. 2M, 20 mL), washed with dichloromethane (20 mL; 2×). The aqueous phase was concentrated under vacuum to give the product (5-bromo-3-chloropyridin-2-yl)methanamine hydrochloride (1.5 g, 4.07 mmol, 44.26% yield, 70% purity, HCl salt) as a light brown solid.

RT 0.18 min (method 2); m/z 222.9 (M+H)⁺ (ESI⁺), 1H NMR (DMSO-d₆, 400 MHz): 8.78 (d, J=2.0 Hz, 1H), 8.69 (br, 3H), 8.47 (d, J=2.0 Hz, 1H), 4.24 (d, J=6.2 Hz, 2H).

### Preparation of Intermediate 149.2

### ethyl 2-(((5-bromo-3-chloropyridin-2-yl)methyl)amino)-2-oxoacetate

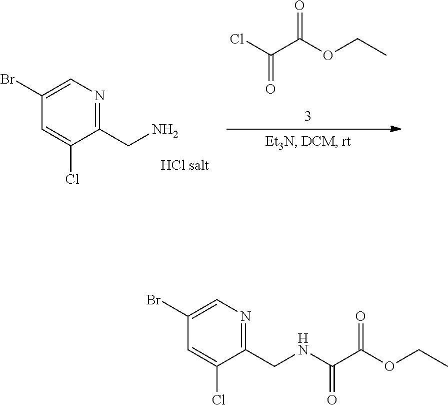

To a mixture of (5-bromo-3-chloro-2-pyridyl)methanamine (1.5 g, 5.82 mmol, HCl salt) in dichloromethane (30 mL) at 0° C. was added DIPEA (2.25 g, 17.45 mmol). Then, ethyl 2-chloro-2-oxoacetate (952.77 mg, 6.98 mmol) was added over 5 min and the mixture was stirred at 0° C. for 30 min. The mixture was warmed to 20° C. and stirred for 30 min at this temperature. The mixture was quenched with aqueous NaHCO₃ solution (50 mL) and extracted with dichloromethane (50 mL). The organic phase was separated, dried over Na₂SO₄, filtered and concentrated under vacuum. The residue was purified by column chromatography on silica gel (Petroleum ether:Ethyl acetate=10:1 to 1:1) to give the product ethyl 2-(((5-bromo-3-chloropyridin-2-yl)methyl)amino)-2-oxoacetate (1300 mg, 3.64 mmol, 62.57% yield, 65.6% purity) as a white solid.

RT 0.61 min (method 1); m/z 322.8 (M+H)⁺ (ESI⁺).

### Preparation of Intermediate 149.3

### Ethyl 6-bromo-8-chloroimidazo[1,5-a]pyridine-3-carboxylate

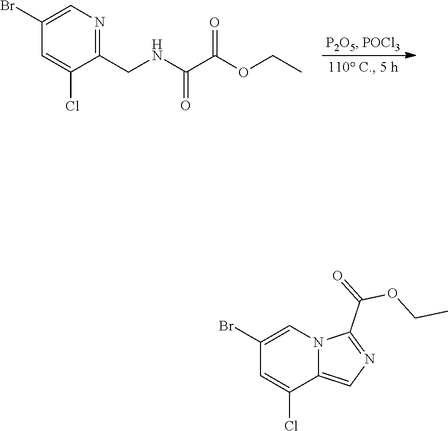 [SMILES: CCOC(=O)C(=O)NCc1ncc(Br)cc1Cl ; CCOC(=O)c1ncc2c(Cl)cc(Br)cn12]

To a mixture of ethyl 2-(((5-bromo-3-chloropyridin-2-yl)methyl)amino)-2-oxoacetate (1300 mg, 4.04 mmol) in POCl₃ (15 mL) at 0° C. was added phosphorus pentoxide (2.87 g, 20.21 mmol). The mixture was heated to 110° C. and stirred for 5 h at this temperature. The mixture was cooled to 25° C. and concentrated under vacuum. The residue was dissolved in ethyl acetate (50 mL, washed with water (30 mL) and saturated NaHCO₃ solution (30 mL). Then, it was finally concentrated under vacuum. The residue was purified by flash silica gel chromatography (ISCO®; 4 g SepaFlash® Silica Flash Column, Eluent of 0˜30% Ethyl acetate/Petroleum ether gradient @30 mL/min) to give the product ethyl 6-bromo-8-chloroimidazo[1,5-a]pyridine-3-carboxylate (900 mg, 2.97 mmol, 73.34% yield) as a white solid.

RT 0.718 min (method 1), m/z 304.8 (M+H)⁺ (ESI⁺), 1H NMR (CDCl₃, 400 MHz): 9.47 (s, 1H), 7.77 (s, 1H), 7.20 (s, 1H), 4.65-4.42 (m, 2H), 1.57-1.42 (m, 3H).

### Preparation of Intermediate 149.4

### 6-bromo-8-chloroimidazo[1,5-a]pyridine-3-carboxamide

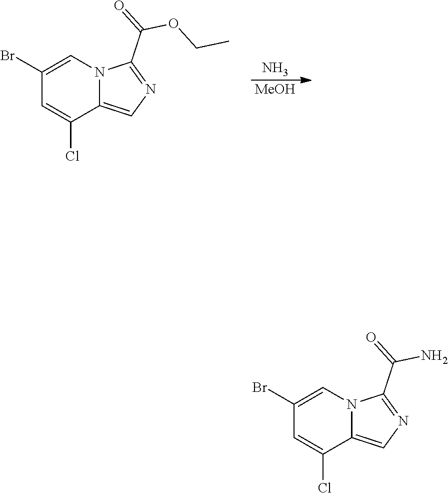

A solution of ethyl 6-bromo-8-chloro-imidazo[1,5-a]pyridine-3-carboxylate (3 g, 9.88 mmol) in NH₃/MeOH (7 M, 59.30 mL) was stirred at 25° C. for 16 h. The mixture was filtered and washed with MeOH (5 mL) to give the product 6-bromo-8-chloroimidazo[1,5-a]pyridine-3-carboxamide (2.6 g, 9.47 mmol, 95.83% yield) as a white solid.

RT 0.45 min (method 3); m/z 275.9 (M+H)⁺ (ESI⁺); ¹H NMR (CDCl₃, 400 MHz) δ: 9.62 (s, 1H), 7.66 (s, 1H), 7.32 (s, 1H), 7.16 (s, 1H), 5.55 (s, 1H).

### Preparation of Intermediate 149.5

### 6-bromo-8-chloroimidazo[1,5-a]pyridine-3-carbothioamide

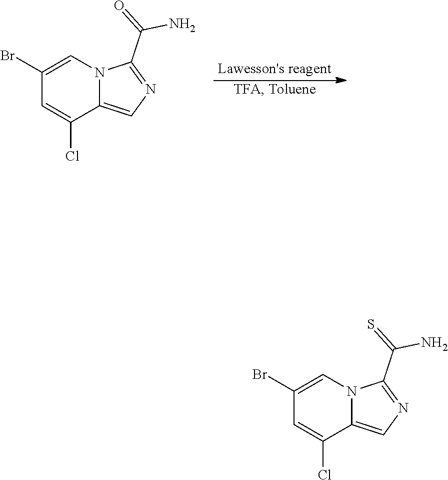 [SMILES: NC(=O)c1ncc2c(Cl)cc(Br)cn12 ; "Lawesson's reagent" ; "TFA, Toluene" ; NC(=S)c1ncc2c(Cl)cc(Br)cn12]

To a suspension of 6-bromo-8-chloro-imidazo[1,5-a]pyridine-3-carboxamide (4 g, 14.57 mmol) in toluene (40 mL) was added TFA (1.66 g, 14.57 mmol, 1.08 mL). The reaction mixture was stirred at 25° C. for 1 h. The reaction mixture was concentrated under reduced pressure and the resulting residue was diluted with toluene (40 mL). Lawesson's reagent (17.68 g, 43.72 mmol) was added and the reaction mixture was stirred at 110° C. for 16 h. The mixture was concentrated under reduced pressure and the residue was triturated with MeOH (60 mL) at 50° C. for 1 h. After filtration, the filtrate cake was collected, dried under reduce pressure to give the product 6-bromo-8-chloroimidazo[1,5-a]pyridine-3-carbothioamide (3.94 g, 12.84 mmol, 88.10% yield, 94.68% purity) as a brown solid.

¹H NMR (DMSO-d₆, 400 MHz) δ: 10.76 (s, 1H), 9.84 (s, 1H), 9.71 (s, 1H), 7.84 (s, 1H), 7.65 (s, 1H).

### Preparation of Intermediate 149.6

### ethyl 2-(6-bromo-8-chloroimidazo[1,5-a]pyridin-3-yl)thiazole-5-carboxylate

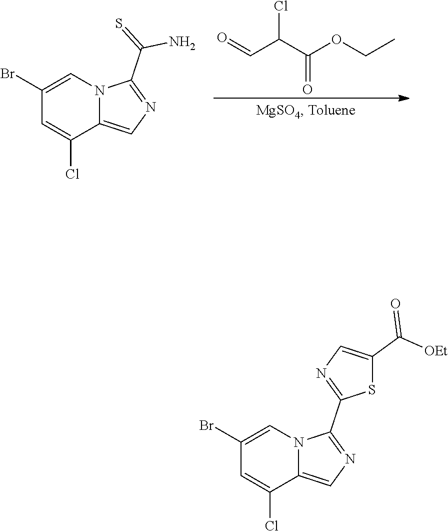

To the solution of ethyl 2-chloro-3-oxo-propanoate (4.77 g, 31.67 mmol) in toluene (15 mL) was added 6-bromo-8-chloro-imidazo[1,5-a]pyridine-3-carbothioamide (2 g, 6.88 mmol) and MgSO₄ (1.66 g, 13.77 mmol). The mixture was stirred at 100° C. for 16 h, then filtered and the filtrate was concentrated under reduced pressure. The residue was triturated in MeOH (20 mL) at 17° C. for 8 min and the precipitate was filtered, dried under reduce pressure to give the product ethyl 2-(6-bromo-8-chloroimidazo[1,5-a]pyridin-3-yl)thiazole-5-carboxylate (2 g, 4.52 mmol, 87.424% yield) as a brown solid.

RT 0.702 min; m/z 385.8 (M+H)⁺ (ESI⁺); ¹H NMR (CDCl₃, 400 MHz) 9.74 (s, 1H), 8.44 (s, 1H), 7.72 (s, 1H), 7.11 (s, 1H), 4.41-4.36 (q, J=7.2 Hz, 2H), 1.41-1.37 (t, J=7.2 Hz, 3H).

### Preparation of Intermediate 149.7

### (2-(6-bromo-8-chloroimidazo[1,5-a]pyridin-3-yl)thiazol-5-yl)methanol

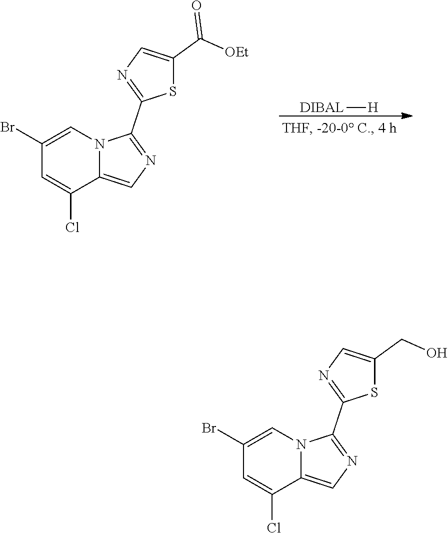

To a solution of ethyl 2-(6-bromo-8-chloro-imidazo[1,5-a]pyridin-3-yl)thiazole-5-carboxylate (1000 mg, 2.59 mmol) in THE (30 mL) was added dropwise DIBAL-H (1 M, 10.35 mL) at −20° C. under N₂ over 6 min. The reaction mixture was stirred at −20° C. for 2 h and at 0° C. for another 2 hr. The reaction mixture was quenched with NH₄Cl (aq., sat., 4 mL) and MeOH (4 mL), then filtered through silica gel and washed with THF (20 mL; 3×). The filtrate was collected, dried over Na₂SO₄ and concentrated under reduced pressure. The residue was triturated in MeOH (15 mL). The precipitate was filtered and dried under vacuum to give the product [2-(6-bromo-8-chloro-imidazo[1,5-a]pyridin-3-yl)thiazol-5-yl]methanol (490 mg, 1.42 mmol, 54.98% yield, 100% purity) as a yellow solid.

RT 0.450 min (method 4); m/z 345.9 (M+H)⁺ (ESI⁺); ¹H NMR (CDCl₃, 400 MHz) 9.75 (s, 1H), 7.80 (s, 1H), 7.71 (s, 1H), 7.06 (s, 1H), 5.96 (s, 2H).

### Preparation of Intermediate 149.8

### 2-(6-bromo-8-chloroimidazo[1,5-a]pyridin-3-yl)thiazole-5-carbaldehyde

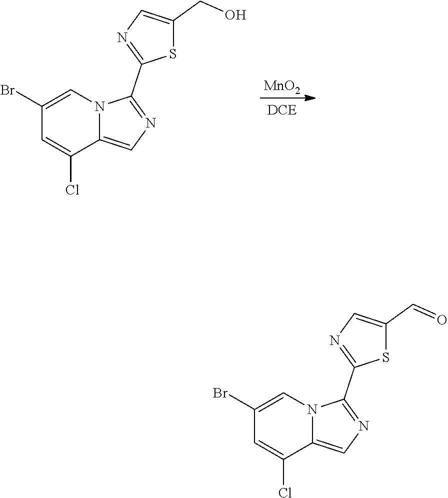 [SMILES: OCc1cnc(-c2ncc3c(Cl)cc(Br)cn23)s1 ; "MnO2" ; O=Cc1cnc(-c2ncc3c(Cl)cc(Br)cn23)s1]

To a solution of [2-(6-bromo-8-chloro-imidazo[1,5-a]pyridin-3-yl)thiazol-5-yl]methanol (500 mg, 1.45 mmol) in 1,2-dichloroethane (7 mL) was added MnO₂ (1.26 g, 14.51 mmol). The reaction mixture was stirred at 80° C. for 20 min, filtered and the cake was washed with DCM (20 mL, 3×). The filtrate was collected, dried over Na₂SO₄, concentrated under reduced pressure to give the product 2-(6-bromo-8-chloro-imidazo[1,5-a]pyridin-3-yl)thiazole-5-carbaldehyde (327 mg, 931.66 μmol, 64.21% yield, 97.610% purity) as a yellow solid.

RT 0.495 min (method 4); m/z 343.9 (M+H)⁺ (ESI⁺); ¹H NMR (CDCl₃, 400 MHz) δ: 10.01 (s, 1H), 9.83 (s, 1H), 8.48 (s, 1H), 7.81 (s, 1H), 7.18 (s, 1H).

### Preparation of Intermediate 149.9

### 2-(6-bromo-8-chloroimidazo[1,5-a]pyridin-3-yl)-5-(difluoromethyl)thiazole

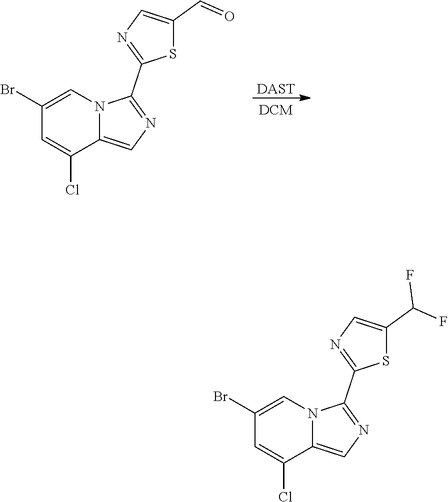 [SMILES: O=Cc1cnc(-c2ncc3c(Cl)cc(Br)cn23)s1 ; "DAST" ; FC(F)c1cnc(-c2ncc3c(Cl)cc(Br)cn23)s1]

To a solution of 2-(6-bromo-8-chloro-imidazo[1,5-a]pyridin-3-yl)thiazole-5-carbaldehyde (327 mg, 954.47 μmol) in DCM (15 mL) was added dropwise a solution of DAST (1.54 g, 9.54 mmol, 1.26 mL) in DCM (0.6 mL) at −15° C. under N₂. The mixture was stirred at −15° C. for 2 h, at 0° C. for another 2 h and at 20° C. for another 3 h. The mixture was quenched by NH₄Cl (aq., sat., 5 mL) at −15° C., diluted with water (40 mL) and extracted with DCM (20 mL, 3×). The organic phase was separated and the combined organic layer was dried over Na₂SO₄, concentrated under pressure to give the product 2-(6-bromo-8-chloro-imidazo[1,5-a]pyridin-3-yl)-5-(difluoromethyl)thiazole (360 mg, crude) as a yellow solid.

RT 0.542 min (method 4); m/z 366.0 (M+H)⁺ (ESI⁺); ¹H NMR (CDCl₃, 400 MHz) δ: 9.73-9.72 (m, 1H), 8.05-8.04 (t, J=2.0 Hz, 1H), 7.75 (m, 1H), 7.12-6.84 (m, 2H).

### Preparation of Intermediate 149.10

### 2-(6-(benzylthio)-8-chloroimidazo[1,5-a]pyridin-3-yl)-5-(difluoromethyl)thiazole

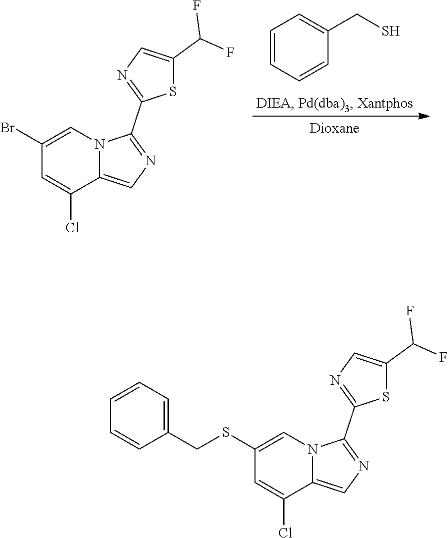

To a solution of 2-(6-bromo-8-chloro-imidazo[1,5-a]pyridin-3-yl)-5-(difluoromethyl)thiazole (234 mg, 510.59 μmol, 79.555% purity), Pd₂(dba)₃ (46.76 mg, 51.06 μmol) and Xantphos (29.54 mg, 51.06 μmol) in 1,4-dioxane (4 mL) was added DIEA (197.97 mg, 1.53 mmol, 266.80 μL) and phenylmethanethiol (63.42 mg, 510.59 μmol, 59.83 μL) under N₂. The mixture was degassed with N₂ (3×) and stirred at 100° C. for 1 h under a nitrogen atmosphere. The mixture was filtered and the filtrate was concentrated under reduced pressure. The residue was purified by preparative TLC (Petroleum ether:Ethyl acetate=10:1) to give the product 2-(6-benzylsulfanyl-8-chloro-imidazo[1,5-a]pyridin-3-yl)-5-(difluoromethyl)thiazole (140 mg, 338.10 μmol, 66.22% yield, 98.504% purity) as a yellow solid.

RT 0.595 min (method 4); m/z 408.1 (M+H)⁺ (ESI⁺); ¹H NMR (CDCl₃, 400 MHz) 9.43 (s, 1H), 8.00 (s, 1H), 7.68 (s, 1H), 7.34-7.30 (m, 5H), 6.97 (t, J=55.2 Hz, 1H), 6.90 (s, 1H), 4.15 (s, 2H).

### Preparation of Intermediate 149.11

### 2-(6-(benzylthio)-8-chloro-1-iodoimidazo[1,5-a]pyridin-3-yl)-5-(difluoromethyl)thiazole

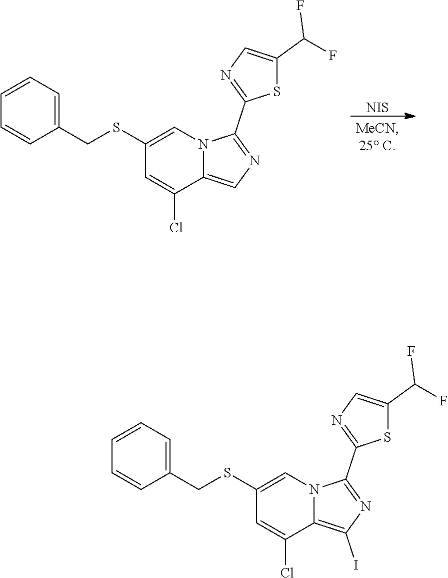

To a solution of 2-(6-benzylsulfanyl-8-chloro-imidazo[1,5-a]pyridin-3-yl)-5-(difluoromethyl)thiazole (74 mg, 181.42 μmol) in MeCN (3 mL) was added NIS (40.82 mg, 181.42 μmol). The mixture was stirred at 25° C. for 16 h, then AcOH (32.68 mg, 544.27 μmol, 31.13 μL) was added at 25° C. and the mixture was stirred for another 1 h. The reaction mixture of 2-(6-(benzylthio)-8-chloro-1-iodoimidazo[1,5-a]pyridin-3-yl)-5-(difluoromethyl)thiazole was used for next step without purification.

RT 0.766 min (method 3); m/z 533.9 (M+H)⁺ (ESI⁺).

### Preparation of Intermediate 149.12

### 8-chloro-3-(5-(difluoromethyl)thiazol-2-yl)-1-iodoimidazo[1,5-a]pyridine-6-sulfonyl chloride

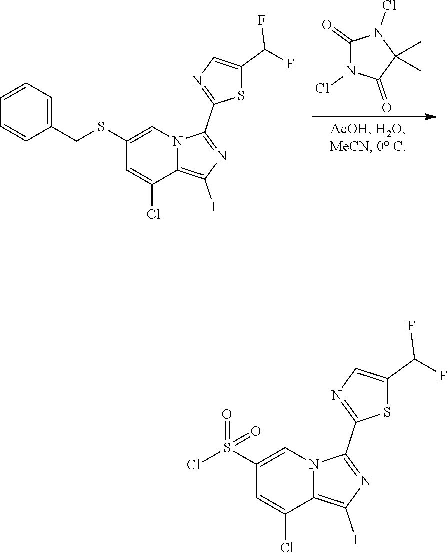 [SMILES: FC(F)c1cnc(-c2nc(I)c3c(Cl)cc(SCc4ccccc4)cn23)s1 ; CC1(C)C(=O)N(Cl)C(=O)N1Cl ; O=S(=O)(Cl)c1cc(Cl)c2c(I)nc(-c3ncc(C(F)F)s3)n2c1]

At 0° C., to the previous suspension of 2-(6-(benzylthio)-8-chloro-1-iodoimidazo[1,5-a]pyridin-3-yl)-5-(difluoromethyl)thiazole (78 mg, 146.13 μmol, theoretical amount) in MeCN (3 mL) were successively added AcOH (17.55 mg, 292.25 μmol, 16.71 μL), H₂O (5.27 mg, 292.25 μmol, 5.27 μL) and 1,3-dichloro-5,5-dimethyl-imidazolidine-2,4-dione (57.58 mg, 292.25 μmol). The mixture was stirred at 0° C. for 1 h. The reaction mixture of 8-chloro-3-(5-(difluoromethyl)thiazol-2-yl)-1-iodoimidazo[1,5-a]pyridine-6-sulfonyl chloride (74.35 mg, theoretical weight) was used for next step directly without purification.

RT 0.684 min (method 3); m/z 509.8 (M+H)⁺ (ESI⁺);

### Preparation of Example 149

### 8-chloro-N-(1-cyanocyclopropyl)-3-(5-(difluoromethyl)thiazol-2-yl)-1-iodoimidazo[1,5-a]pyridine-6-sulfonamide

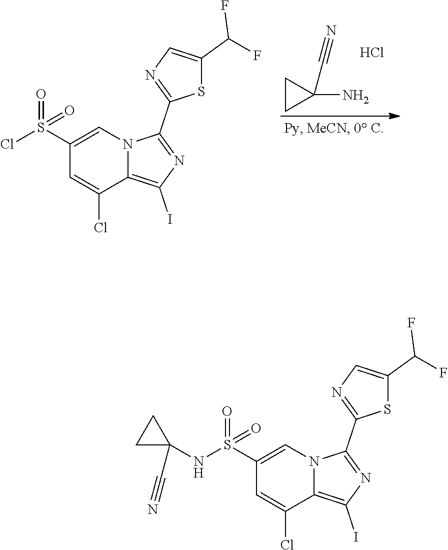

To a solution of 1-aminocyclopropane-1-carbonitrile (51.98 mg, 438.38 μmol, HCl salt) in pyridine (2.94 g, 37.17 mmol, 3 mL) was added dropwise the reaction mixture containing 8-chloro-3-(5-(difluoromethyl)thiazol-2-yl)-1-iodoimidazo[1,5-a]pyridine-6-sulfonyl chloride (74.35 mg, crude, theoretical weight) at 0° C. for 3 min. The mixture was stirred at 0° C. for 1 h, quenched with water (5 mL) and extracted with EtOAc (15 mL; 3×). The organic layers were separated, combined, dried over Na₂SO₄ and concentrated under reduced pressure. The residue was purified by preparative TLC (Petroleum ether:Ethyl acetate=2:1) to give the product 8-chloro-N-(1-cyanocyclopropyl)-3-[5-(difluoromethyl)thiazol-2-yl]-1-iodo-imidazo[1,5-a]pyridine-6-sulfonamide (64 mg, 97.79 μmol, 66.92% yield, 84.915% purity) as a yellow solid.

RT 0.611 min (method 4); m/z 555.8 (M+H)⁺ (ESI⁺); ¹H NMR (CDCl₃, 400 MHz) δ: 10.37 (s, 1H), 8.11 (s, 1H), 7.32-7.31 (d, J=1.2 Hz, 1H), 7.13 (s, 1H), 7.13-6.85 (t, J=55.6 Hz, 1H), 1.67-1.62 (m, 2H), 1.27-1.24 (m, 2H).

### Preparation of Example 150

### 8-chloro-N-(1-cyanocyclopropyl)-3-(5-(difluoromethyl)thiazol-2-yl)imidazo[1,5-a]pyridine-6-sulfonamide

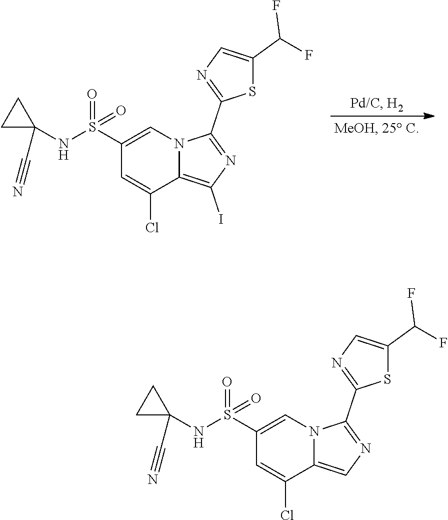 [SMILES: N#CC1(NS(=O)(=O)c2cc(Cl)c3c(I)nc(-c4ncc(C(F)F)s4)n3c2)CC1 ; N#CC1(NS(=O)(=O)c2cc(Cl)c3cnc(-c4ncc(C(F)F)s4)n3c2)CC1]

To a solution of 8-chloro-N-(1-cyanocyclopropyl)-3-[5-(difluoromethyl)thiazol-2-yl]-1-iodo-imidazo[1,5-a]pyridine-6-sulfonamide (50 mg, 89.97 μmol) in MeOH (3 mL) was added Pd/C (50.00 mg, 46.98 μmol, 10% purity). The reaction was degassed with H₂ (balloon, 15 psi, 3×) and stirred at 25° C. for 1 h. The mixture was filtered, washed with MeOH (3 mL; 2×) and the filtrate was concentrated under reduced pressure to give the product 8-chloro-N-(1-cyanocyclopropyl)-3-[5-(difluoromethyl)thiazol-2-yl]imidazo[1,5-a]pyridine-6-sulfonamide (26 mg, 28.43 μmol, 31.60% yield) as a yellow solid.

RT 0.558 min (method 3); m/z 430.1 (M+H)⁺ (ESI⁺);

### Preparation of Example 151

### 4-(6-(N-(1-cyanocyclopropyl)sulfamoyl)-3-(5-(difluoromethyl)thiazol-2-yl)imidazo[1,5-a]pyridin-8-yl)-N,N-dimethylpiperazine-1-carboxamide

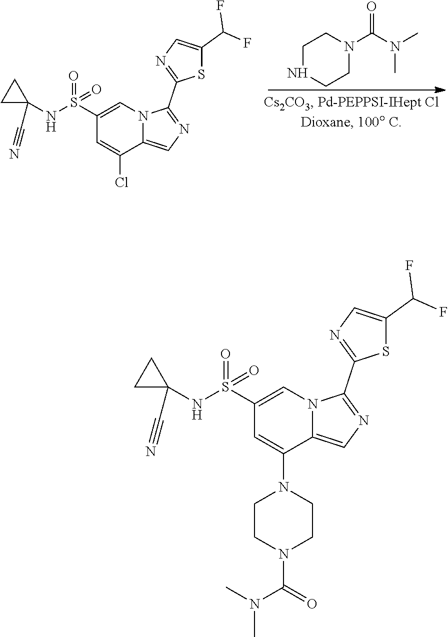 [SMILES: N#CC1(NS(=O)(=O)c2cc(Cl)c3cnc(-c4ncc(C(F)F)s4)n3c2)CC1 ; CN(C)C(=O)N1CCNCC1 ; CN(C)C(=O)N1CCN(c2cc(S(=O)(=O)NC3(C#N)CC3)cn3c(-c4ncc(C(F)F)s4)ncc23)CC1]

To a solution of N,N-dimethylpiperazine-1-carboxamide (10.97 mg, 69.79 μmol) and 8-chloro-N-(1-cyanocyclopropyl)-3-[5-(difluoromethyl)thiazol-2-yl]imidazo[1,5-a]pyridine-6-sulfonamide (15 mg, 34.90 μmol) in dioxane (0.5 mL) was added Cs₂CO₃ (22.74 mg, 69.79 μmol) and Pd-PEPPSI-IPentCl o-picoline (1.70 mg, 1.74 μmol) under N₂ and the mixture was stirred at 100° C. for 5 h. The mixture was filtered and the filtrate was concentrated under reduced pressure. The residue was purified by preparative TLC (Petroleum ether:Ethyl acetate=0:1) to give an impure product, which was further purified by preparative-HPLC (column: Phenomenex Synergi C18 150*25 mm*10 μm; mobile phase: A: 0.225% formic acid in water, B: MeCN; B %: 40%-70%, 10 min) and lyophilized directly to give the product 4-(6-(N-(1-cyanocyclopropyl)sulfamoyl)-3-(5-(difluoromethyl)thiazol-2-yl)imidazo[1,5-a]pyridin-8-yl)-N,N-dimethylpiperazine-1-carboxamide (4.98 mg, 8.16 μmol, 23.38% yield) as a yellow solid.

RT 0.552 min (method 3); m/z 551.1 (M+H)⁺ (ESI⁺); ¹H NMR (400 MHz, DMSO-d₆) 9.67 (s, 1H), 8.42 (s, 1H), 8.32 (s, 1H), 7.90 (s, 1H), 7.50 (t, J=54.8 Hz, 1H), 6.61 (s, 1H), 3.38-3.36 (m, 4H), 3.35-3.30 (m, 4H), 2.80 (s, 6H), 1.33-1.26 (m, 2H), 1.25-1.18 (m, 2H).

### Preparation of Example 152

### 3-(5-(difluoromethyl)-1,3,4-thiadiazol-2-yl)-8-(hydroxymethyl)-N-(1-methylcyclopropyl)imidazo[1,5-a]pyridine-6-sulfonamide

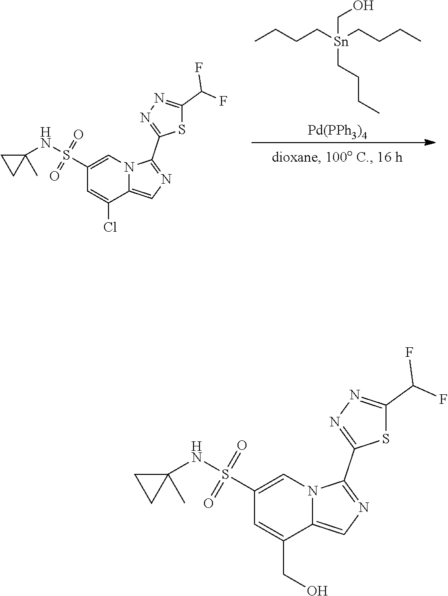 [SMILES: CC1(NS(=O)(=O)c2cc(Cl)c3cnc(-c4nnc(C(F)F)s4)n3c2)CC1 ; CCC[CH2][Sn]([CH2]O)([CH2]CCC)[CH2]CCC ; CC1(NS(=O)(=O)c2cc(CO)c3cnc(-c4nnc(C(F)F)s4)n3c2)CC1 ; "Pd(PPh3)4"]

To a solution of 8-chloro-3-(5-(difluoromethyl)-1,3,4-thiadiazol-2-yl)-N-(1-methylcyclopropyl)imidazo[1,5-a]pyridine-6-sulfonamide (20 mg, 47.64 μmol) and (tributylstannyl)methanol (152.95 mg, 476.35 μmol) in dioxane (1 mL) was added Pd(PPh₃)₄ (5.50 mg, 4.76 μmol). The mixture was degassed with N₂ (3×) and stirred at 100° C. for 16 h under N₂ atmosphere. The reaction mixture was cooled to 20° C., filtered and the filtrate was concentrated under vacuum. The residue was first purified by preparative TLC (petroleum ether:ethyl acetate=0:1), then further purified by preparative HPLC (column: Unisil 3-100 C18 Ultra 150*25 mm*5 μm; mobile phase: A: 0.225% formic acid in water, B: MeCN; B %: 30%-50%, 10 min) and lyophilized directly to give the product 3-(5-(difluoromethyl)-1,3,4-thiadiazol-2-yl)-8-(hydroxymethyl)-N-(1-methylcyclopropyl)imidazo[1,5-a]pyridine-6-sulfonamide (20 mg, 28.89 μmol, 60.64% yield, 60% purity) as a yellow solid.

RT 0.443 min (method 1); m/z 415.9 (M+H)⁺ (ESI⁺)

### Preparation of Intermediate 152.1

### 8-(((tert-butyldimethylsilyl)oxy)methyl)-3-(5-(difluoromethyl)-1,3,4-thiadiazol-2-yl)-N-(1-methylcyclopropyl)imidazo[1,5-a]pyridine-6-sulfonamide

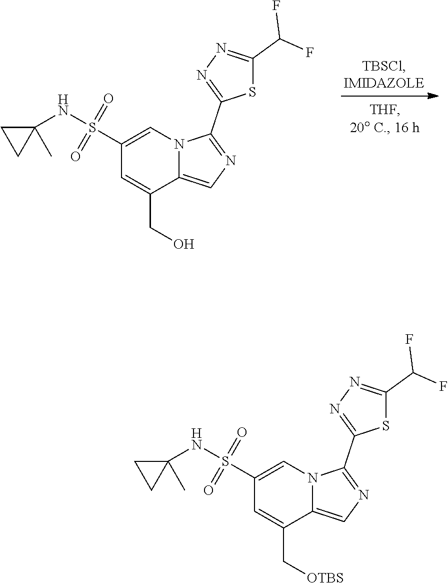 [SMILES: CC1(NS(=O)(=O)c2cc(CO)c3cnc(-c4nnc(C(F)F)s4)n3c2)CC1 ; CC1(NS(=O)(=O)c2cc(CO[Si](C)(C)C(C)(C)C)c3cnc(-c4nnc(C(F)F)s4)n3c2)CC1]

To a solution of 3-(5-(difluoromethyl)-1,3,4-thiadiazol-2-yl)-8-(hydroxymethyl)-N-(1-methylcyclopropyl)imidazo[1,5-a]pyridine-6-sulfonamide (15 mg, 21.66 μmol, 60% purity) and TBSCl (6.53 mg, 43.33 μmol, 5.31 μL) in THE (1 mL) was added imidazole (2.95 mg, 43.33 μmol). The mixture was stirred at 20° C. for 16 h, then diluted with EtOAc (20 mL) and washed with water (30 mL; 3×). The organic layer was collected, dried over Na₂SO₄, filtered and concentrated under vacuum. The residue was purified by preparative TLC (petroleum ether:ethyl acetate=5:1) to give the product 8-(((tert-butyldimethylsilyl)oxy)methyl)-3-(5-(difluoromethyl)-1,3,4-thiadiazol-2-yl)-N-(1-methylcyclopropyl)imidazo[1,5-a]pyridine-6-sulfonamide (15 mg, 21.24 μmol, 98.04% yield, 75% purity) as a yellow solid.

RT 0.631 min (method 1); m/z 530.1 (M+H)⁺ (ESI⁺).

### Alternative Preparation of Example 152

### 3-(5-(difluoromethyl)-1,3,4-thiadiazol-2-yl)-8-(hydroxymethyl)-N-(1-methylcyclopropyl)imidazo[1,5-a]pyridine-6-sulfonamide

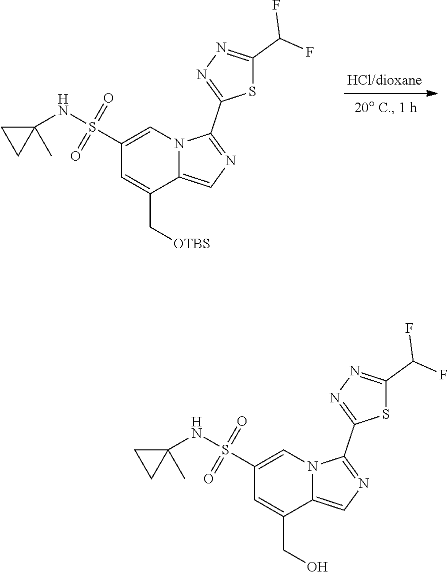 [SMILES: CC1(NS(=O)(=O)c2cc(CO[Si](C)(C)C(C)(C)C)c3cnc(-c4nnc(C(F)F)s4)n3c2)CC1 ; CC1(NS(=O)(=O)c2cc(CO)c3cnc(-c4nnc(C(F)F)s4)n3c2)CC1]

A solution of 8-(((tert-butyldimethylsilyl)oxy)methyl)-3-(5-(difluoromethyl)-1,3,4-thiadiazol-2-yl)-N-(1-methylcyclopropyl)imidazo[1,5-a]pyridine-6-sulfonamide (15 mg, 28.32 μmol) in HCl/dioxane (4 M, 3.00 mL) was stirred at 20° C. for 1 h. The reaction mixture was concentrated under vacuum to give a residue which was triturated with MeOH (0.5 mL) at 20° C. for 1 h. After filtration, the cake was collected, dried under vacuum to give the product 3-(5-(difluoromethyl)-1,3,4-thiadiazol-2-yl)-8-(hydroxymethyl)-N-(1-methylcyclopropyl)imidazo[1,5-a]pyridine-6-sulfonamide (8.14 mg, 18.24 μmol, 64.42% yield, 93.1% purity) as a yellow solid.

RT 0.405 min (method 1); m/z 416.0 (M+H)⁺ (ESI⁺); ¹H NMR (DMSO-d₆, 400 MHz): 9.82 (s, 1H), 8.56 (s, 1H), 7.98 (s, 1H), 7.69 (t, J=53.4 Hz, 1H), 7.41 (d, J=1.4 Hz, 1H), 5.80 (t, J=5.6 Hz, 1H), 4.85 (d, J=5.6 Hz, 2H), 1.19 (s, 3H), 0.70 (t, J=5.6 Hz, 2H), 0.46 (dd, J=6.4 Hz, 4.8 Hz, 2H).

### Preparation of Intermediate 153

### 3-(5-(difluoromethyl)-1,3,4-thiadiazol-2-yl)-N-(1-(fluoromethyl)cyclopropyl)imidazo[1,5-a]pyridine-6-sulfonamide

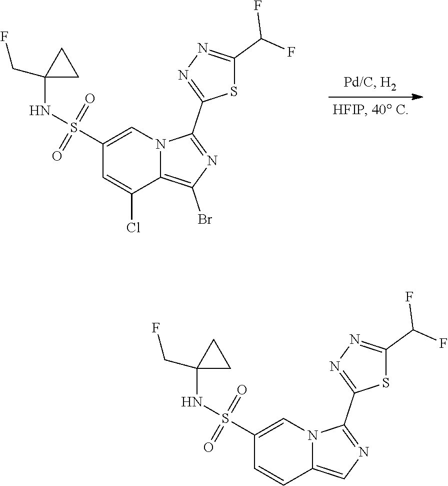 [SMILES: O=S(=O)(NC1(CF)CC1)c1cc(Cl)c2c(Br)nc(-c3nnc(C(F)F)s3)n2c1 ; O=S(=O)(NC1(CF)CC1)c1ccc2cnc(-c3nnc(C(F)F)s3)n2c1]

3 batches were conducted in parallel and combined for the work-up

To a mixture of 1-bromo-8-chloro-3-(5-(difluoromethyl)-1,3,4-thiadiazol-2-yl)-N-(1-(fluoromethyl)cyclopropyl)imidazo[1,5-a]pyridine-6-sulfonamide (500 mg, 967.60 μmol) in HFIP (15 mL) and THE (15 mL) were added Pd/C (160 mg, 10% purity) and DIEA (250.11 mg, 1.94 mmol, 337.08 μL). The reaction mixture was degassed with H₂ (15 psi, 3×) and stirred at 40° C. for 16 h. The 3 reaction mixtures were combined, diluted with EtOAc (50 mL), filtered and concentrated under vacuum. The resulting residue was dissolved in EtOAc (50 mL). The organic layer was washed with NaHCO₃ (aq., sat., 30 mL) and brine (30 mL), The organic phase was dried over Na₂SO₄, filtered and concentrated under vacuum to give 1.15 g of a crude mixture.

50 mg of this crude residue were purified by preparative TLC (petroleum ether:ethyl acetate=3:1) to give the product 3-(5-(difluoromethyl)-1,3,4-thiadiazol-2-yl)-N-(1-(fluoromethyl)cyclopropyl)imidazo[1,5-a]pyridine-6-sulfonamide (11.4 mg, 28.26 μmol, 24.75% yield, 100% purity) as a white solid.

RT 0.44 min (method 1); m/z 403.9 (M+H)⁺ (ESI⁺); ¹H NMR (CDCl₃, 400 MHz): 10.20 (s, 1H), 7.81 (s, 1H), 7.98 (s, 1H), 7.76 (dd, J=9.6 Hz, 0.8 Hz, 3H), 7.36 (dd, J=10.8 Hz, 1.2 Hz, 1H), 7.09 (t, J=53.6 Hz, 1H), 5.50 (s, 15H), 4.29 (d, J=48.4 Hz), 1.15-1.11 (m, 2H), 0.90-0.87 (dd, J=6.4 Hz, 4.8 Hz, 2H).

Compounds listed in the table below were prepared according to the corresponding general procedures or when stated in a similar way to related compound and starting from the corresponding intermediates.

| Cpd | | Yield | | | |
|---|---|---|---|---|---|
| number | Structure | (%) | Procedure | LC/MS | 1H NMR |
| 154 | 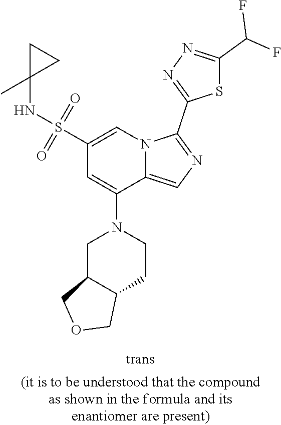 [SMILES: CC1(NS(=O)(=O)c2cc(N3CC[C@@H]4COC[C@H]4C3)c3cnc(-c4nnc(C(F)F)s4)n3c2)CC1] | 35.39% | Procedure 1 | RT 0.472 min (method 3); m/z 511.0 (M + H)⁺ (ESI⁺); | ¹H NMR (DMSO-d₆, 400 MHz): 9.53 (s, 1H), 8.40 (s, 1H), 7.95 (s, 1H), 7.67 (t, J = 52.8 Hz, 1H), 6.62 (s, 1H), 3.88- 3.78 (m, 2H), 3.66- 3.62 (m, 2H), 3.54- 3.41 (m, 2H), 3.35- 3.33 (m, 2H), 2.63- 2.59 (m, 1H), 2.45- 2.43 (m, 1H), 1.98- 1.93 (m, 1H), 1.82- 1.76 (m, 1H), 1.15 (s, 3H), 0.73-0.70 (m, 2H), 0.46-0.44 (m, 2H) |
| 155 | 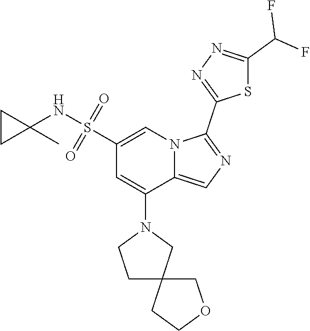 [SMILES: CC1(NS(=O)(=O)c2cc(N3CCC4(CCOC4)C3)c3cnc(-c4nnc(C(F)F)s4)n3c2)CC1] | 16.91% | Procedure 1 | RT 0.469 min (method 3); m/z 510.9 (M + H)⁺ (ESI⁺) | ¹H NMR (DMSO-d₆, 400 MHz): 9.38 (s, 1H), 8.33 (br s, 1H), 8.18 (s, 1H), 7.67 (t, J = 53.4 Hz, 1H), 6.15 (s, 1H), 3.85 (t, J = 6.8 Hz, 2H), 3.81-3.76 (m, 2H), 3.73-3.65 (m, 4H), 2.10-2.04 (m, 2H), 2.02-.93 (m, 2H), 1.18 (s, 3H), 0.74 (dd, J = 6.0 Hz, 4.8 Hz, 2H), 0.44 (dd, J = 6.4 Hz, 4.4 Hz, 2H) |
| 156 | 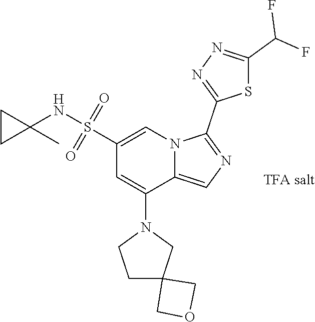 [SMILES: CC1(NS(=O)(=O)c2cc(N3CCC4(COC4)C3)c3cnc(-c4nnc(C(F)F)s4)n3c2)CC1] | 39.4% | Procedure 1 | RT 0.861 min (method 1), m/z 497.8 (M + H)⁺ (ESI⁺) | ¹H NMR (DMSO-d₆, 400 MHz): 9.38 (s, 1H), 8.33 (s, 1H), 8.19 (s, 1H), 7.65 (t, J = 53.2 Hz, 1H), 6.15 (s, 1H), 4.67 (d, J = 6.0 Hz, 2H), 4.57 (d, J = 6.0 Hz, 2H), 3.95 (s, 2H), 3.68 (t, J = 7.2 Hz, 2H), 2.35 (t, J = 6.8 Hz, 2H), 1.19 (s, 3H), 0.73 (t, J = 5.6 Hz, 2H), 0.47-0.42 (m, 2H). |
| 157 | 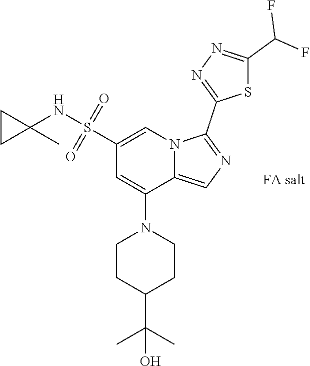 [SMILES: CC1(NS(=O)(=O)c2cc(N3CCC(C(C)(C)O)CC3)c3cnc(-c4nnc(C(F)F)s4)n3c2)CC1] | 7.16% | Procedure 1 | RT 0.474 min (method 1), m/z 527.0 (M + H)⁺ (ESI⁺) | ¹H NMR (DMSO-d₆, 400 MHz): 9.56 (s, 1H), 8.38 (br s, 1H), 8.36 (s, 1H), 7.94 (s, 1H), 7.67 (t, J = 53.2 Hz, 1H), 6.66 (s, 1H), 4.18 (br s, 1H), 3.84 (d, J = 12.0 Hz, 2H), 2.83 (t, J = 11.2 Hz, 2H), 1.87 (d, J = 5.2 Hz, 2H), 1.53 (d, J = 5.2 Hz, 2H), 1.50- 1.48 (m, 1H), 1.15 (s, 3H), 1.10 (s, 6H), 0.72 (s, 2H), 0.44 (s, 2H) |
| 158 | 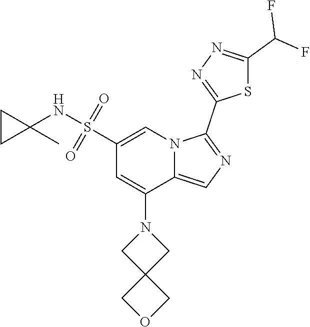 [SMILES: CC1(NS(=O)(=O)c2cc(N3CC4(COC4)C3)c3cnc(-c4nnc(C(F)F)s4)n3c2)CC1] | 7.64% | Procedure 1 | RT 0.443 min (method 1), m/z 483.0 (M + H)⁺ (ESI⁺) | ¹H NMR (DMSO-d₆, 400 MHz): 9.36 (s, 1H), 8.39 (s, 1H), 7.94 (s, 1H), 7.67(t, J = 52.8 Hz, 1H), 6.05 (s, 1H), 4.78 (s, 4H), 4.45 (s, 4H), 1.17 (s, 3H), 0.71 (t, J = 6.8 Hz, 2H), 0.46-0.42 (m, 2H) |
| 159 | 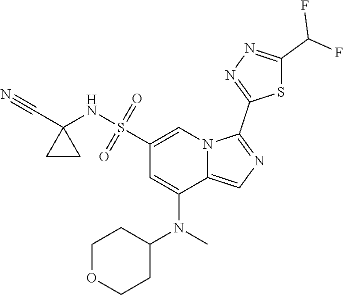 [SMILES: CN(c1cc(S(=O)(=O)NC2(C#N)CC2)cn2c(-c3nnc(C(F)F)s3)ncc12)C1CCOCC1] | 20.44% | Procedure 1 | RT 0.369 min (method 2); m/z 510.2 (M + H)⁺ (ESI⁺) | ¹H NMR (DMSO-d₆, 400 MHz): 9.58 (s, 1H), 8.00 (s, 1H), 7.68 (t, J = 52.0 1H), 6.56 (s, 1H), 4.10- 4.05 (m, 1H), 4.04- 3.92 (m, 2H), 3.44- 3.41 (m, 2H), 2.95 (s, 3H), 1.94-1.90 (m, 2H), 1.74-1.71 (m, 2H), 1.44-1.43 (m, 2H), 1.38-1.37 (m, 2H). |
| 160 | 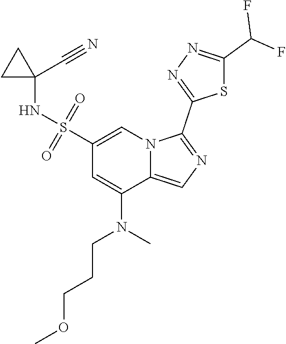 [SMILES: COCCCN(C)c1cc(S(=O)(=O)NC2(C#N)CC2)cn2c(-c3nnc(C(F)F)s3)ncc12] | 13.86% | Procedure 1 | RT 0.440 min (method 1); m/z 498.0 (M + H)⁺ (ESI⁺) | ¹H NMR (DMSO-d₆, 400 MHz): 9.55 (s, 1H), 8.12 (s, 1H), 7.68 (t, J = 52.0 Hz, 1H), 6.38 (s, 1H), 3.63 (t, J = 7.6 Hz, 1H), 3.41 (t, J = 6.0 Hz, 2H), 3.24 (s, 3H), 3.15 (s, 3H), 1.94-1.87 (m, 2H), 1.49-1.45 (m, 2H), 1.39-1.35 (m, 2H). |
| 161 | 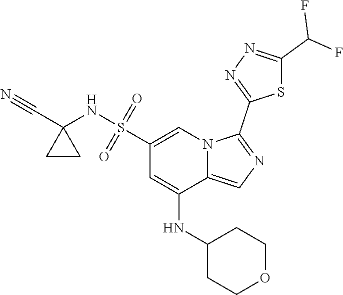 [SMILES: N#CC1(NS(=O)(=O)c2cc(NC3CCOCC3)c3cnc(-c4nnc(C(F)F)s4)n3c2)CC1] | 8.26% | Procedure 1 | RT 0.439 min (method 1); m/z 496.0 (M + H)⁺ (ESI⁺) | ¹H NMR (DMSO-d₆, 400 MHz): 9.34 (s, 1H), 8.20 (s, 1H), 7.67 (t, J = 52.0, 1H), 6.99 (d, J = 4.0 Hz, 1H), 6.30 (s, 1H), 3.96 (d, J = 8.0 Hz, 2H), 3.70-3.67 (m, 1H), 3.48 (t, J = 12.0, 2H), 2.02 (d, J = 14.4 Hz, 2H), 1.64-1.53 (m, 2H), 1.49-1.41 (m, 2H), 1.39-1.32 (m, 2H). |
| 162 | 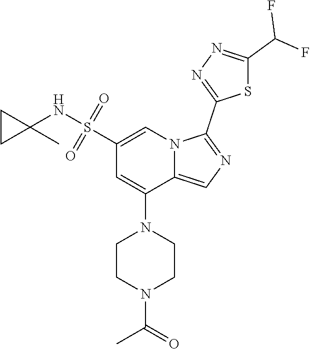 [SMILES: CC(=O)N1CCN(c2cc(S(=O)(=O)NC3(C)CC3)cn3c(-c4nnc(C(F)F)s4)ncc23)CC1] | 23.39% | Procedure 1 | RT 0.438 min (method 1); m/z 512.2 (M + H)⁺ (ESI⁺) | ¹H NMR (DMSO-d₆, 400 MHz): 9.60 (s, 1H), 8.44 (s, 1H), 8.06 (s, 1H), 7.68 (t, J = 53.2, 1H), 6.69 (s, 1H), 3.71 (s, 4H), 3.28 (s, 4H), 2.08 (s, 3H), 1.15 (s, 3H), 0.72 (t, J = 6.0 Hz, 2H), 0.45 (t, J = 6.0 Hz, 2H) |
| 163 | 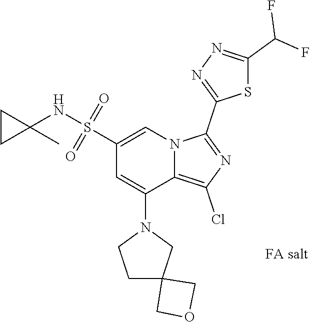 [SMILES: CC1(NS(=O)(=O)c2cc(N3CCC4(COC4)C3)c3c(Cl)nc(-c4nnc(C(F)F)s4)n3c2)CC1] | 14.02% | Procedure 1 | RT 0.490 min (method 1), m/z 531.0 (M + H)⁺ (ESI⁺) | ¹H NMR (DMSO-d₆, 400 MHz): 9.55 (s, 1H), 8.46 (br s, 1H), 8.39 (s, 1H), 7.68 (t, J = 53.6 Hz, 1H), 6.69 (s, 1H), 4.63 (d, J = 6.0 Hz, 2H), 4.58 (d, J = 6.0 Hz, 2H), 3.65 (s, 2H), 2.34 (d, J = 8.4 Hz, 2H), 2.29 (d, J = 7.2 Hz, 2H), 1.17 (s, 3H), 0.74 (t, J = 6.0 Hz, 2H), 0.46 (d, J = 5.2 Hz, 2H) |
| 164 | 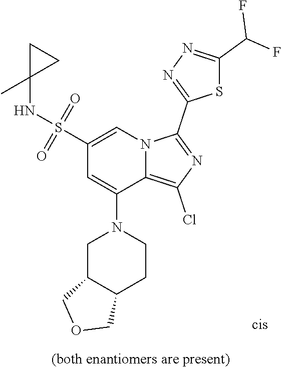 [SMILES: CC1(NS(=O)(=O)c2cc(N3CC[C@@H]4COC[C@@H]4C3)c3c(Cl)nc(-c4nnc(C(F)F)s4)n3c2)CC1] | 3.23% | Procedure 1 | RT 0.506 min (method 1); m/z 544.9 (M + H)⁺ (ESI⁺) | ¹H NMR (DMSO-d₆, 400 MHz): 9.61 (s, 1H), 8.50 (s, 1H), 7.70 (t, J = 56.0 Hz, 1H), 6.87 (s, 1H), 3.84-3.79 (m, 2H), 3.75-3.66 (m, 2H), 3.19-3.11 (m, 1H), 3.02-2.92 (m, 2H), 2.63-2.60 (m, 2H), 2.51-2.43 (m, 1H), 2.03-1.96 (m, 1H), 1.91-1.84 (m, 1H), 1.17 (s, 3H), 0.77- 0.71 (m, 2H), 0.48- 0.45 (m, 2H). |
| 165 | 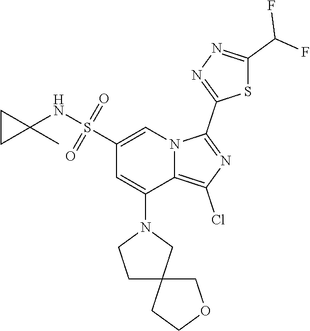 [SMILES: CC1(NS(=O)(=O)c2cc(N3CCC4(CCOC4)C3)c3c(Cl)nc(-c4nnc(C(F)F)s4)n3c2)CC1] | 19.00% | Procedure 1 | RT 0.512 min (method 1), m/z 545.0 (M + H)⁺ (ESI⁺) | ¹H NMR (DMSO-d₆, 400 MHz): 9.54 (s, 1H), 8.43 (s, 1H), 7.68 (t, J = 53.4 Hz, 1H), 6.70 (s, 1H), 3.82-3.77 (m, 2H), 3.67 (d, J = 8.4 Hz, 1H), 3.59 (d, J = 8.4 Hz, 1H), 3.50 (t, J = 6.8 Hz, 2H), 3.44- 3.35 (m, 2H), 2.05- 2.00 (m, 2H), 1.99- 1.88 (m, 2H), 1.17 (s, 3H), 0.74 (t, J = 5.6 Hz, 2H), 0.46 (t, J = 6.0 Hz, 2H) |
| 166 | 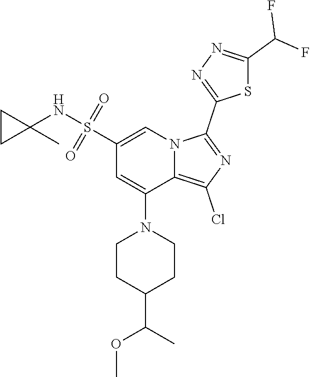 [SMILES: COC(C)C1CCN(c2cc(S(=O)(=O)NC3(C)CC3)cn3c(-c4nnc(C(F)F)s4)nc(Cl)c23)CC1] | 4.93% | Procedure 1 | RT 0.596 min (method 1); m/z 560.9 (M + H)⁺ (ESI⁺) | ¹H NMR (400 MHz, DMSO-d₆): 9.59 (d, J = 1.0 Hz, 1H), 8.51 (s, 1H), 7.68 (t, J = 53.2 Hz, 1H), 6.79 (s, 1H), 3.48 (br d, J = 12.4 Hz, 2H), 3.27 (s, 3H), 3.18- 3.15 (m, 1H), 2.72- 2.68 (m, 2H), 1.91 (br d, J = 7.6 Hz 1H), 1.73 (br d, J = 9.6 Hz, 1H), 1.64-1.56 (m, 2H), 1.55 (br d, J = 1.2 Hz, 1H), 1.17 (s, 3H), 1.10 (d, J = 6.0 Hz, 3H), 0.74- 0.72 (m 2H), 0.47- 0.44 (m, 2H). |

### Preparation of Example 168

### oxetan-3-yl 8-(4-(dimethylcarbamoyl)piperazin-1-yl)-6-(N-(1-methylcyclopropyl)sulfamoyl)imidazo[1,2-a]pyridine-3-carboxylate

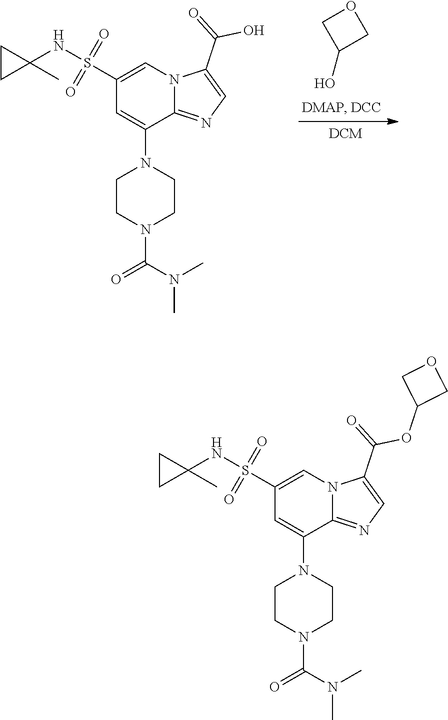 [SMILES: CN(C)C(=O)N1CCN(c2cc(S(=O)(=O)NC3(C)CC3)cn3c(C(=O)O)cnc23)CC1 ; OC1COC1 ; CN(C)C(=O)N1CCN(c2cc(S(=O)(=O)NC3(C)CC3)cn3c(C(=O)OC4COC4)cnc23)CC1]

A solution of 8-(4-(dimethylcarbamoyl)piperazin-1-yl)-6-(N-(1-methylcyclopropyl)sulfamoyl)imidazo[1,2-a]pyridine-3-carboxylic acid (10 mg, 22.20 μmol), oxetan-3-ol (1.97 mg, 26.64 μmol), DMAP (542.35 μg, 4.44 μmol) and DCC (5.50 mg, 26.64 μmol, 5.39 μL) in DCM (0.5 mL) was stirred at 20° C. for 1 h. The solution was quenched with water (10 mL) and extracted with DCM (10 mL; 3×). The combined organic layer was washed with brine (20 mL), dried over anhydrous Na₂SO₄, filtered and the filtrate was concentrated under reduced pressure. The residue was purified by preparative-HPLC (column: Waters Xbridge C18 150*50 mm*10 μm; mobile phase: A: 10 mmol NH₄HCO₃ in water, B: MeCN; B %: 25%-55%, 10 min) and lyophilized directly to give the product oxetan-3-yl 8-(4-(dimethylcarbamoyl)piperazin-1-yl)-6-(N-(1-methylcyclopropyl)sulfamoyl)imidazo[1,2-a]pyridine-3-carboxylate (0.9 mg, 1.71 μmol, 7.68% yield, 96% purity) as a yellow gum.

RT 0.769 min (method 1); m/z 507.3 (M+H)⁺ (ESI⁺); ¹H NMR (CDCl₃, 400 MHz) 9.46 (s, 1H), 8.34 (s, 1H), 6.94 (s, 1H), 5.76-5.69 (m, 1H), 5.04-5.00 (m, 2H), 4.85-4.81 (m, 2H), 3.62-3.60 (m, 4H), 3.53-3.51 (m, 4H), 2.89 (s, 6H), 1.31 (s, 3H), 0.86-0.83 (m, 2H), 0.57-0.54 (m, 2H).

### Preparation of Intermediate 169.1

### tert-butyl (8-chloro-6-(N-(1-methylcyclopropyl)sulfamoyl)imidazo[1,2-a]pyridin-3-yl)carbamate

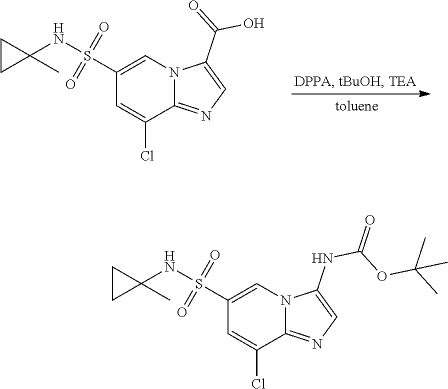 [SMILES: CC1(NS(=O)(=O)c2cc(Cl)c3ncc(C(=O)O)n3c2)CC1 ; CC1(NS(=O)(=O)c2cc(Cl)c3ncc(NC(=O)OC(C)(C)C)n3c2)CC1]

To a solution of 8-chloro-6-(N-(1-methylcyclopropyl)sulfamoyl)imidazo[1,2-a]pyridine-3-carboxylic acid (280 mg, 849.11 μmol) in t-BuOH (2.33 g, 31.37 mmol, 3 mL) and toluene (3 mL) was added TEA (214.80 mg, 2.12 mmol, 295.46 μL) followed by DPPA (467.35 mg, 1.70 mmol, 367.99 μL). The reaction mixture was degassed and purged with N₂ (3×) and the mixture was stirred at 90° C. for 4 h under N₂ atmosphere. The mixture was poured into NaHCO₃ (aq., sat., 10 mL) and extracted with EtOAc (10 mL, 3×). The combined organic layer was dried over Na₂SO₄, filtered and concentrated under vacuum. The residue was purified by flash silica gel chromatography (ISCO®; 12 g SepaFlash® Silica Flash Column, Eluent of 20-50% Ethyl acetate/Petroleum ether gradient @30 mL/min) to give an impure product which was further purified by preparative HPLC (column: Phenomenex luna C18 150*25 mm*10 μm; mobile phase: A: 0.225% formic acid in water, B: MeCN; B %: 34%-64%; 10 min) and lyophilized directly to give the product tert-butyl (8-chloro-6-(N-(1-methylcyclopropyl)sulfamoyl)imidazo[1,2-a]pyridin-3-yl)carbamate (75 mg, 187.09 μmol, 22.03% yield, 100% purity) as a yellow solid.

RT 0.768 min (method 1); m/z 401.1 (M+H)⁺ (ESI⁺); ¹H NMR (DMSO-d₆, 400 MHz): 9.90 (s, 1H), 8.62 (s, 1H), 8.37 (s, 1H), 7.66 (s, 1H), 7.57 (s, 1H), 1.48 (s, 9H), 1.12 (s, 3H), 0.64-0.73 (m, 2H), 0.41-0.50 (m, 2H).

### Preparation of Intermediate 169.2

### tert-butyl (8-(4-(dimethylcarbamoyl)piperazin-1-yl)-6-(N-(1-methylcyclopropyl)sulfamoyl)imidazo[1,2-a]pyridin-3-yl)carbamate

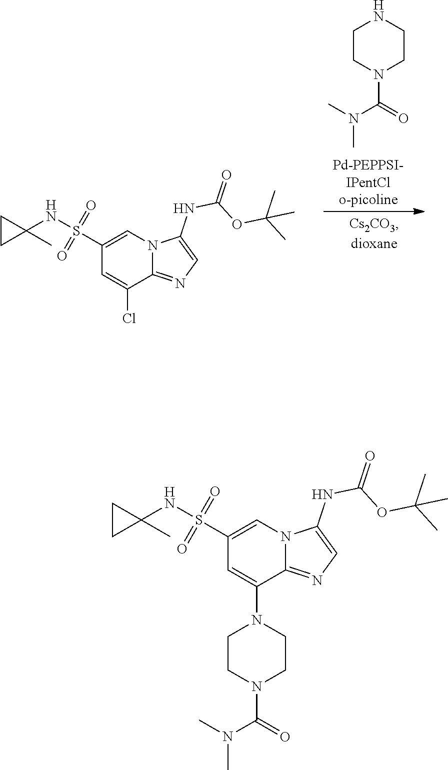 [SMILES: CN(C)C(=O)N1CCNCC1 ; CC1(NS(=O)(=O)c2cc(Cl)c3ncc(NC(=O)OC(C)(C)C)n3c2)CC1 ; CN(C)C(=O)N1CCN(c2cc(S(=O)(=O)NC3(C)CC3)cn3c(NC(=O)OC(C)(C)C)cnc23)CC1]

To a solution of tert-butyl (8-chloro-6-(N-(1-methylcyclopropyl)sulfamoyl)imidazo[1,2-a]pyridin-3-yl)carbamate (75 mg, 187.09 μmol) in dioxane (2 mL) was added Cs₂CO₃ (121.91 mg, 374.18 μmol), N,N-dimethylpiperazine-1-carboxamide (58.83 mg, 374.18 μmol) and Pd-PEPPSI-IPentCl o-picoline (8.05 mg, 9.35 μmol). The reaction mixture was degassed, purged with N₂ (3×) and, then stirred at 100° C. for 4 h under N₂ atmosphere. The mixture was poured into water (5 mL) and extracted with EtOAc (5 mL, 3×). The combined organic layer was dried over Na₂SO₄, filtered and concentrated under reduced pressure. The resulting residue was purified by preparative-HPLC (column: Phenomenex luna C18 150*25 mm*10 μm; mobile phase: A: 0.225% formic acid in water, B: MeCN; B %: 34%-64%; 10 min) and lyophilized directly to give the product tert-butyl (8-(4-(dimethylcarbamoyl)piperazin-1-yl)-6-(N-(1-methylcyclopropyl)sulfamoyl)imidazo[1,2-a]pyridin-3-yl)carbamate (20 mg, 36.52 μmol, 19.52% yield, 95.26% purity) as a white solid.

RT 0.839 min (method 1); m/z 522.3 (M+H)⁺ (ESI⁺); ¹H NMR (CDCl₃, 400 MHz): 8.21 (s, 1H), 7.52 (s, 1H), 6.65 (s, 1H), 6.25 (br, 1H), 4.93 (s, 1H), 3.70-3.58 (m, 4H), 3.57-3.45 (m, 4H), 2.89 (s, 6H), 1.56 (s, 9H), 1.30 (s, 3H), 0.85 (t, J=6 Hz, 2H), 0.85 (t, J=6 Hz, 2H).

### Preparation of Intermediate 169.3

### 4-(3-amino-6-(N-(1-methylcyclopropyl)sulfamoyl)imidazo[1,2-a]pyridin-8-yl)-N,N-dimethylpiperazine-1-carboxamide

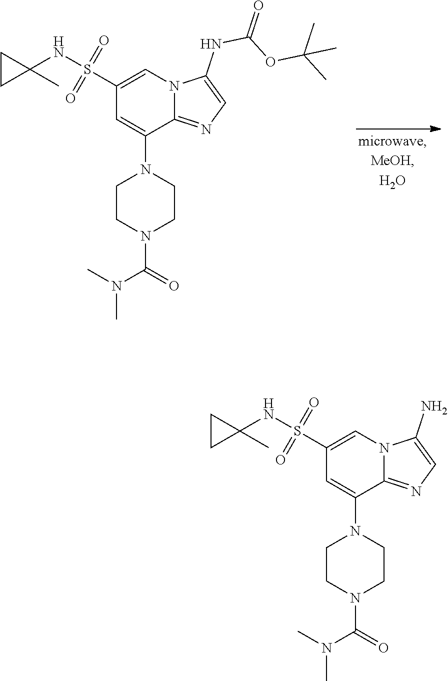 [SMILES: CN(C)C(=O)N1CCN(c2cc(S(=O)(=O)NC3(C)CC3)cn3c(NC(=O)OC(C)(C)C)cnc23)CC1 ; CN(C)C(=O)N1CCN(c2cc(S(=O)(=O)NC3(C)CC3)cn3c(N)cnc23)CC1]

A mixture of tert-butyl (8-(4-(dimethylcarbamoyl)piperazin-1-yl)-6-(N-(1-methylcyclopropyl)sulfamoyl)imidazo[1,2-a]pyridin-3-yl)carbamate (8 mg, 15.34 μmol) in MeOH (0.5 mL) and H₂O (0.5 mL) was stirred at 120° C. for 2 h under microwave, and the reaction mixture was concentrated under reduced pressure. The resulting residue was purified by preparative-HPLC (column: Phenomenex luna C18 150*25 mm*10 μm; mobile phase: A: 0.225% formic acid in water, B: MeCN; B %: 6%-36%; 10 min) and lyophilized directly to give the product 4-(3-amino-6-(N-(1-methylcyclopropyl)sulfamoyl)imidazo[1,2-a]pyridin-8-yl)-N,N-dimethylpiperazine-1-carboxamide (0.85 mg, 2.02 μmol, 13.15% yield, 100% purity) as an off-white solid.

RT 0.616 min (method 1); m/z 422.2 (M+H)⁺ (ESI⁺); ¹H NMR (CDCl₃, 400 MHz): 8.36 (s, 1H), 7.22 (s, 1H), 6.55 (s, 1H), 4.95 (s, 1H), 3.68-3.57 (m, 4H), 3.57-3.46 (m, 4H), 3.18-3.41 (m, 2H), 2.88 (s, 6H), 1.30 (s, 3H), 0.84 (t, J=5.6 Hz, 2H), 0.53 (t, J=6.4 Hz, 2H).

### Preparation of Example 169

### 4-(3-(2-chloroacetamido)-6-(N-(1-methylcyclopropyl)sulfamoyl)imidazo[1,2-a]pyridin-8-yl)-N,N-dimethylpiperazine-1-carboxamide

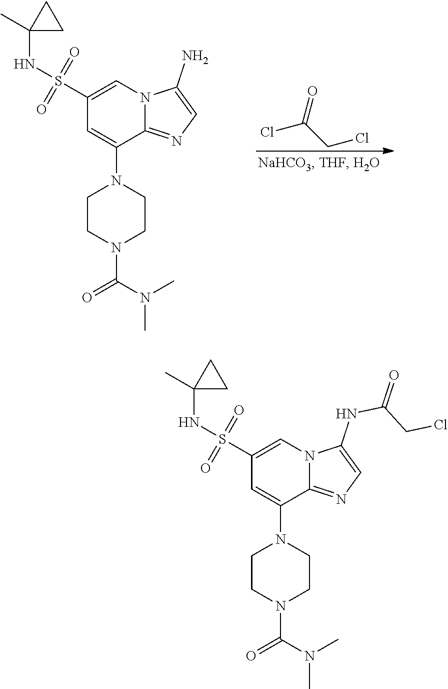

To a solution of 4-[3-amino-6-[(1-methylcyclopropyl)sulfamoyl]imidazo[1,2-a]pyridin-8-yl]-N,N-dimethyl-piperazine-1-carboxamide (10 mg, 0.0237 mmol) and NaHCO₃ (10 mg, 0.119 mmol) in THE (2 mL) and water (1 mL) was added 2-chloroacetyl chloride (4.0 mg, 0.0356 mmol) at 0° C. The reaction mixture was stirred at 20° C. for 2 h and poured into water (5 mL). The aqueous phase was extracted with EtOAc (5 mL, 3×). The combined organic layer was dried over anhydrous Na₂SO₄, filtered and concentrated under reduced pressure. The residue was purified by preparative-HPLC (column: Phenomenex luna C18 150*25 mm*10 μm; mobile phase: A: 0.225% formic acid in water, B: MeCN; B %: 21%-51%; 10 min) to give the product 4-(3-(2-chloroacetamido)-6-(N-(1-methylcyclopropyl)sulfamoyl)imidazo[1,2-a]pyridin-8-yl)-N,N-dimethylpiperazine-1-carboxamide (11 mg, 0.0218 mmol, 91.99% yield, 98.8% purity) as a white solid.

RT 0.709 min (method 1); m/z 498.2 (M+H)⁺ (ESI⁺); ¹H NMR (CDCl₃, 400 MHz): 8.37 (br, 1H), 8.08 (s, 1H), 7.59 (s, 1H), 6.68 (s, 1H), 5.08 (s, 1H), 4.35 (s, 2H), 3.65-3.55 (m, 4H), 3.55-3.45 (m, 4H), 2.89 (s, 6H), 1.30 (s, 3H), 0.84 (s, 2H), 0.55 (s, 2H).

### Preparation of Example 170

### 4-(3-(2-cyanoacetamido)-6-(N-(1-methylcyclopropyl)sulfamoyl)imidazo[1,2-a]pyridin-8-yl)-N,N-dimethylpiperazine-1-carboxamide

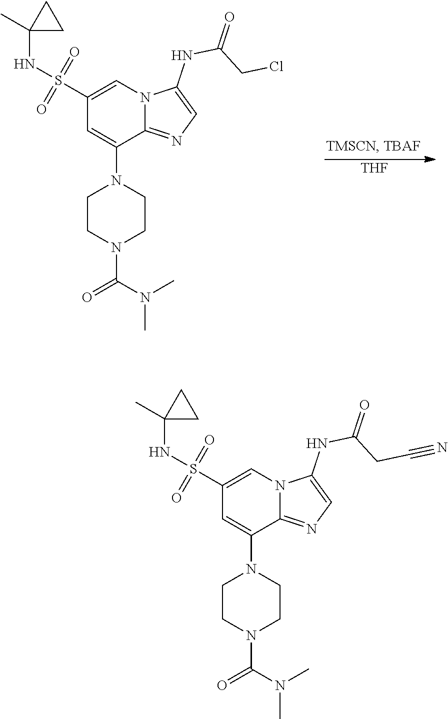 [SMILES: CN(C)C(=O)N1CCN(c2cc(S(=O)(=O)NC3(C)CC3)cn3c(NC(=O)CCl)cnc23)CC1 ; CN(C)C(=O)N1CCN(c2cc(S(=O)(=O)NC3(C)CC3)cn3c(NC(=O)CC#N)cnc23)CC1 ; "THF"]

To a solution of 4-(3-(2-chloroacetamido)-6-(N-(1-methylcyclopropyl)sulfamoyl)imidazo[1,2-a]pyridin-8-yl)-N,N-dimethylpiperazine-1-carboxamide (9.0 mg, 0.0181 mmol) and trimethylsilylformonitrile (5.4 mg, 0.0542 mmol) in THE (2 mL) was added TBAF (0.054 mL, 0.0542 mmol). The reaction mixture was stirred at 25° C. for 16 h, then poured into water (5 mL) and extracted with EtOAc (5 mL, 3×). The combined organic layer was dried with anhydrous Na₂SO₄, filtered and concentrated under reduced pressure. The residue was purified by preparative-HPLC (column: Phenomenex luna C18 150*25 mm*10 μm; mobile phase: A: 0.225% formic acid in water, B: MeCN; B %: 17%-47%; 10 min) to give the product 4-(3-(2-cyanoacetamido)-6-(N-(1-methylcyclopropyl)sulfamoyl)imidazo[1,2-a]pyridin-8-yl)-N,N-dimethylpiperazine-1-carboxamide (2.4 mg, 0.00475 mmol, 26.26% yield, 96.2% purity) as an off white solid.

RT 0.765 min (method 1); m/z 489.2 (M+H)⁺ (ESI⁺); ¹H NMR (CDCl₃, 400 MHz): 9.23 (br s, 1H), 8.26 (s, 1H), 7.62 (s, 1H), 6.64 (s, 1H), 5.62 (s, 1H), 3.80 (s, 2H), 3.55-3.40 (m, 8H), 2.89 (s, 6H), 1.26 (s, 3H), 0.83 (t, J=5.6 Hz, 2H), 0.52 (t, J=5.2 Hz, 2H).

### Preparation Intermediate 171.1

### tert-butyl N-[8-[4-(dimethylcarbamoyl)piperazin-1-yl]-3-iodo-imidazo[1,2-a]pyridin-6-yl]sulfonyl-N-(1-methylcyclopropyl)carbamate

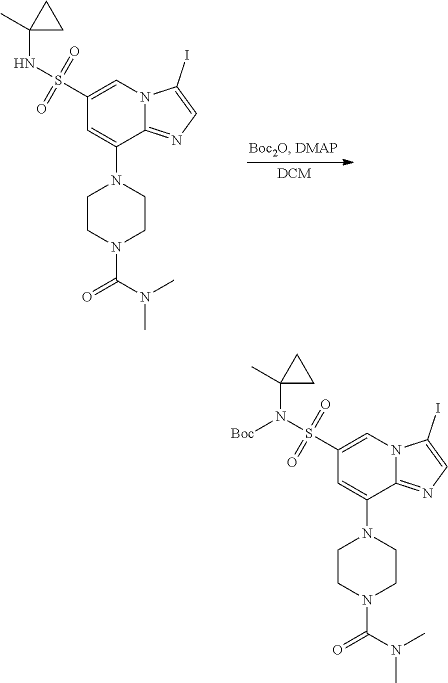 [SMILES: CN(C)C(=O)N1CCN(c2cc(S(=O)(=O)NC3(C)CC3)cn3c(I)cnc23)CC1 ; CN(C)C(=O)N1CCN(c2cc(S(=O)(=O)N(C(=O)OC(C)(C)C)C3(C)CC3)cn3c(I)cnc23)CC1]

To a mixture of 4-(3-iodo-6-(N-(1-methylcyclopropyl)sulfamoyl)imidazo[1,2-a]pyridin-8-yl)-N,N-dimethylpiperazine-1-carboxamide (2.1 g, 3.94 mmol) in DCM (30 mL) was added Boc₂O (1.29 g, 5.92 mmol, 1.36 mL) and DMAP (96.38 mg, 788.88 μmol). The reaction mixture was stirred at 20° C. for 2 h. The reaction mixture was concentrated under reduced pressure. The residue was purified by column chromatography (SiO₂, Petroleum ether/Ethyl acetate=1/1 to 0/1) followed by preparative-HPLC (column: Waters Xbridge C18 150*50 mm*10 um; mobile phase: A: 0.225% NH₄HCO₃ in water, B: MeCN; B %: 48%-68%; 10 min) and lyophilized to give the product tert-butyl ((8-(4-(dimethylcarbamoyl)piperazin-1-yl)-3-iodoimidazo[1,2-a]pyridin-6-yl)sulfonyl)(1-methylcyclopropyl)carbamate (1.1 g, 1.74 mmol, 44.00% yield, 99.8% purity) as a white solid.

RT 0.807 min (method 1); m/z 633.1 (M+H)⁺ (ESI⁺); ¹H NMR (DMSO-d₆, 400 MHz): 8.35 (s, 1H), 7.85 (s, 1H), 6.59 (s, 1H), 3.62-3.50 (m, 4H), 3.30 (s, 4H), 2.78 (s, 6H), 1.52 (s, 3H), 1.28 (s, 9H), 1.01-0.96 (m, 4H).

### Preparation of Intermediate 171.2

### tert-butyl ((8-(4-(dimethylcarbamoyl)piperazin-1-yl)-3-(1-ethoxyvinyl)imidazo[1,2-a]pyridin-6-yl)sulfonyl)(1-methylcyclopropyl)carbamate

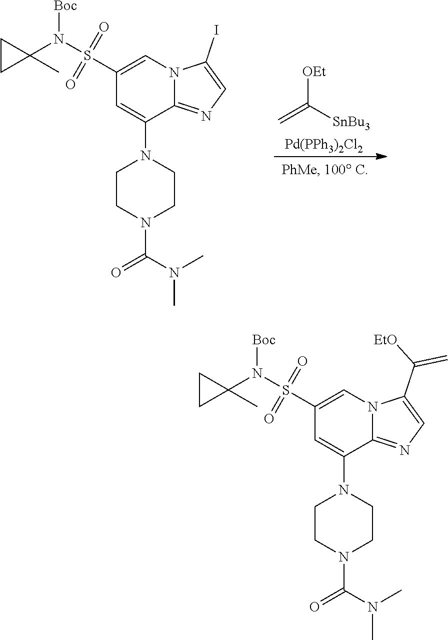

A mixture of tert-butyl ((8-(4-(dimethylcarbamoyl)piperazin-1-yl)-3-iodoimidazo[1,2-a]pyridin-6-yl)sulfonyl)(1-methylcyclopropyl)carbamate (20 mg, 31.62 μmol), tributyl(1-ethoxyvinyl)stannane (13.70 mg, 37.94 μmol, 12.81 μL) and Pd(PPh₃)₄ (3.65 mg, 3.16 μmol) in dry toluene (0.5 mL) was stirred at 100° C. for 2 h under N₂. The reaction mixture was concentrated under reduced pressure to give crude product tert-butyl ((8-(4-(dimethylcarbamoyl)piperazin-1-yl)-3-(1-ethoxyvinyl)imidazo[1,2-a]pyridin-6-yl)sulfonyl)(1-methylcyclopropyl)carbamate (18 mg, 31.21 μmol, 98.71% yield) as a yellow oil which was directly used in the next step without any further purification.

RT 0.662 min (Method 5); m/z 577.2 (M+H)⁺ (ESI⁺).

### Preparation of Example 171

### 4-(3-acetyl-6-(N-(1-methylcyclopropyl)sulfamoyl)imidazo[1,2-a]pyridin-8-yl)-N,N-dimethylpiperazine-1-carboxamide

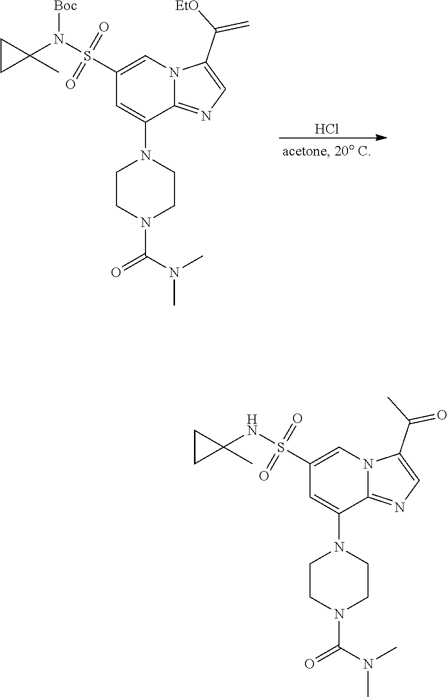 [SMILES: C=C(OCC)c1cnc2c(N3CCN(C(=O)N(C)C)CC3)cc(S(=O)(=O)N(C(=O)OC(C)(C)C)C3(C)CC3)cn12 ; Cl ; CC(=O)c1cnc2c(N3CCN(C(=O)N(C)C)CC3)cc(S(=O)(=O)NC3(C)CC3)cn12]

To a solution of tert-butyl ((8-(4-(dimethylcarbamoyl)piperazin-1-yl)-3-(1-ethoxyvinyl)imidazo[1,2-a]pyridin-6-yl)sulfonyl)(1-methylcyclopropyl)carbamate (18 mg, 31.21 μmol) in acetone (0.5 mL) was added HCl (12 N, 13.00 μL). The mixture was stirred at 20° C. for 1 h, then diluted with H₂O (30 mL) and extracted with EtOAc (20 mL, 2×). The combined organic layer was washed with brine (30 mL, 2×), dried over anhydrous Na₂SO₄, filtered and concentrated undervacuum. The residue was purified by preparative HPLC (column: Unisil 3-100 C18 Ultra 150*50 mm*3 μm; mobile phase: A: 0.225% formic acid in water, B: MeCN; B %: 30%-60%, 7 min) and lyophilized directly to give the product 4-(3-acetyl-6-(N-(1-methylcyclopropyl)sulfamoyl)imidazo[1,2-a]pyridin-8-yl)-N,N-dimethylpiperazine-1-carboxamide (3.61 mg, 7.63 μmol, 24.44% yield, 94.77% purity) as a yellow solid.

RT 0.820 min (method 1); m/z 449.2 (M+H)⁺ (ESI⁺); ¹H NMR (CDCl₃, 400 MHz) 9.83 (d, J=1.2 Hz, 1H), 8.30 (s, 1H), 7.01 (d, J=1.2 Hz, 1H), 5.05 (s, 1H), 3.57-3.66 (m, 4H), 3.49-3.57 (m, 4H), 2.90 (s, 6H), 2.64 (s, 3H), 1.33 (s, 3H), 0.81-0.92 (m, 2H), 0.53-0.61 (m, 2H).

### Preparation of Intermediate 172.1

### tert-butyl ((3-acetyl-8-(4-(dimethylcarbamoyl)piperazin-1-yl)imidazo[1,2-a]pyridin-6-yl)sulfonyl)(1-methylcyclopropyl)carbamate

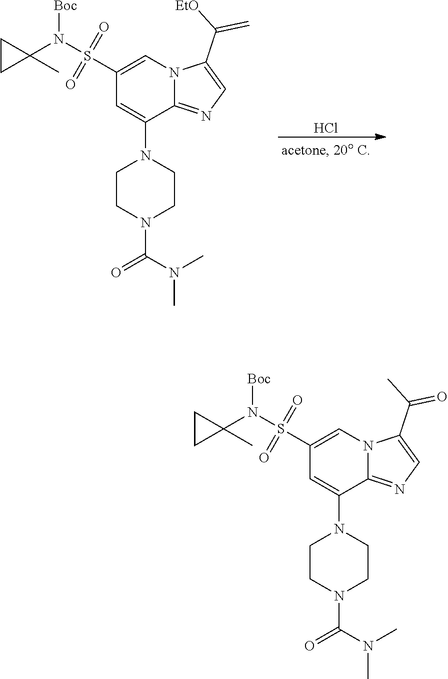 [SMILES: C=C(OCC)c1cnc2c(N3CCN(C(=O)N(C)C)CC3)cc(S(=O)(=O)N(C(=O)OC(C)(C)C)C3(C)CC3)cn12 ; Cl ; CC(=O)c1cnc2c(N3CCN(C(=O)N(C)C)CC3)cc(S(=O)(=O)N(C(=O)OC(C)(C)C)C3(C)CC3)cn12]

To a solution of tert-butyl ((8-(4-(dimethylcarbamoyl)piperazin-1-yl)-3-(1-ethoxyvinyl)imidazo[1,2-a]pyridin-6-yl)sulfonyl)(1-methylcyclopropyl)carbamate (50 mg, 86.70 μmol) in acetone (0.5 mL) was added HCl (1 N, 433.50 μL). The mixture was stirred at 20° C. for 1 h, then diluted with H₂O (20 mL) and extracted with EtOAc (20 mL, 2×). The combined organic layer was washed with brine (20 mL, 2×), dried over anhydrous Na₂SO₄, filtered, concentrated under reduced pressure. The residue was purified by preparative TLC (DCM/MeOH=10/1) to give the product tert-butyl ((3-acetyl-8-(4-(dimethylcarbamoyl)piperazin-1-yl)imidazo[1,2-a]pyridin-6-yl)sulfonyl)(1-methylcyclopropyl)carbamate (40 mg, 72.91 μmol, 84.09% yield) as a yellow solid.

RT 0.846 min (method 1); m/z 549.2 (M+H)⁺ (ESI⁺).

### Preparation of Intermediate 172.2

### tert-butyl ((8-(4-(dimethylcarbamoyl)piperazin-1-yl)-3-(1-hydroxyethyl)imidazo[1,2-a]pyridin-6-yl)sulfonyl)(1-methylcyclopropyl)carbamate

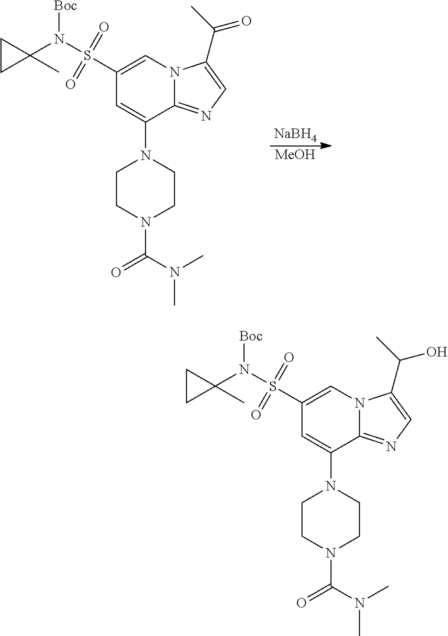 [SMILES: "MeOH" ; CC(=O)c1cnc2c(N3CCN(C(=O)N(C)C)CC3)cc(S(=O)(=O)N(C(=O)OC(C)(C)C)C3(C)CC3)cn12 ; "NaBH4" ; CC(O)c1cnc2c(N3CCN(C(=O)N(C)C)CC3)cc(S(=O)(=O)N(C(=O)OC(C)(C)C)C3(C)CC3)cn12]

To a solution of tert-butyl ((3-acetyl-8-(4-(dimethylcarbamoyl)piperazin-1-yl)imidazo[1,2-a]pyridin-6-yl)sulfonyl)(1-methylcyclopropyl)carbamate (30 mg, 54.68 μmol) in THE (0.2 mL) and MeOH (0.2 mL) was added NaBH₄ (10.34 mg, 273.40 μmol). The resulting mixture was stirred at 20° C. for 1 h, then diluted with H₂O (20 mL) and extracted with EtOAc (20 mL, 2×). The combined organic layer was washed with brine (20 mL, 2×), dried over anhydrous Na₂SO₄, filtered and concentrated under vacuum give the product tert-butyl ((8-(4-(dimethylcarbamoyl)piperazin-1-yl)-3-(1-hydroxyethyl)imidazo[1,2-a]pyridin-6-yl)sulfonyl)(1-methylcyclopropyl)carbamate (30 mg, 54.48 μmol, 99.63% yield) as a yellow solid.

RT 0.581 min (Method 5); m/z 551.3 (M+H)⁺ (ESI⁺).

### Preparation of Example 172

### 4-(3-(1-hydroxyethyl)-6-(N-(1-methylcyclopropyl)sulfamoyl)imidazo[1,2-a]pyridin-8-yl)-N,N-dimethylpiperazine-1-carboxamide

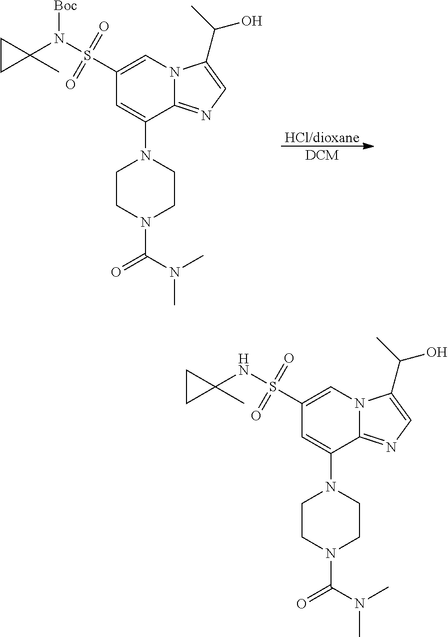

To a solution of tert-butyl ((8-(4-(dimethylcarbamoyl)piperazin-1-yl)-3-(1-hydroxyethyl)imidazo[1,2-a]pyridin-6-yl)sulfonyl)(1-methylcyclopropyl)carbamate (8 mg, 14.53 μmol) in DCM (0.3 mL) was added HCl/dioxane (4 M, 240.00 μL). The mixture was stirred at 20° C. for 1 h, then diluted with H₂O (20 mL) and extracted with EtOAc (20 mL, 2×). The combined organic layer was washed with brine (20 mL, 2×), dried over anhydrous Na₂SO₄, filtered and concentrated under vacuum. The residue was purified by preparative HPLC (column: Phenomenex Luna C18 150*25 mm*10 μm; mobile phase: A: 0.225% formic acid in water, B: MeCN; B %: 12%-42%, 10 min) and lyophilized directly to give the product 4-(3-(1-hydroxyethyl)-6-(N-(1-methylcyclopropyl)sulfamoyl)imidazo[1,2-a]pyridin-8-yl)-N,N-dimethylpiperazine-1-carboxamide (1.93 mg, 4.16 μmol, 28.64% yield, 97.131% purity) as a yellow solid.

RT 0.705 min (method 1); m/z 451.2 (M+H)⁺ (ESI⁺); ¹H NMR (CDCl₃, 400 MHz) 8.65 (d, J=1.2 Hz, 1H), 7.53 (s, 1H), 6.68 (s, 1H), 5.20-5.27 (m, 1H), 4.97 (s, 1H), 3.53-3.63 (m, 4H), 3.46-3.53 (m, 5H), 2.88 (s, 6H), 1.80 (d, J=6.4 Hz, 3H), 1.30 (s, 3H), 0.81-0.88 (m, 2H), 0.50-0.56 (m, 2H).

### Preparation of Example 173

### 3-(5-(difluoromethyl)-1,3,4-thiadiazol-2-yl)-8-(4-isobutyrylpiperazin-1-yl)-N-(1-methylcyclopropyl)imidazo[1,2-a]pyridine-6-sulfonamide

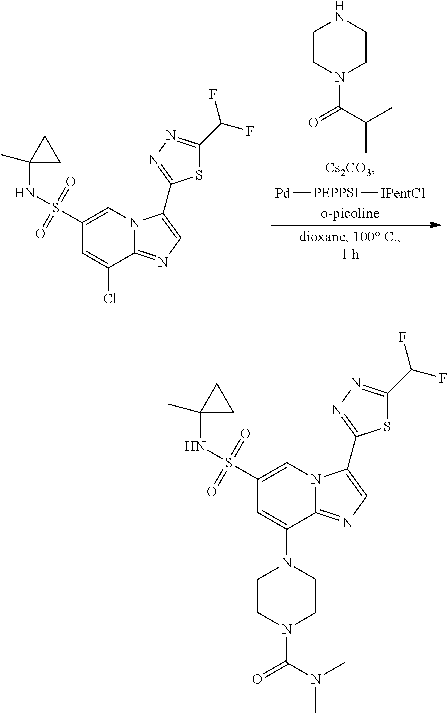

To a solution of 8-chloro-3-(5-(difluoromethyl)-1,3,4-thiadiazol-2-yl)-N-(1-methylcyclopropyl)imidazo[1,2-a]pyridine-6-sulfonamide (30 mg, 0.0715 mmol) in dioxane (0.5 mL) was added 2-methyl-1-(piperazin-1-yl)propan-1-one (17 mg, 0.107 mmol), Cs₂CO₃ (47 mg, 0.143 mmol) at room temperature. Then, to this solution was added Pd-PEPPSI-IPentCl o-picoline (3.5 mg, 0.00357 mmol) in glove box. The mixture was stirred at 100° C. for 1 h outside of the glove box under Ar. The reaction mixture was cooled to room temperature, diluted with H₂O (20 mL) and extracted with EtOAc (20 mL, 2×). The combined organic layer was washed with brine (20 mL, 2×), dried over anhydrous Na₂SO₄, filtered and concentrated under vacuum. The residue was purified by preparative HPLC (column: Phenomenex Synergi C18 150*25 mm*10 μm; mobile phase: A: 0.225% formic acid in water, B: MeCN; B %: 40%-70%, 10 min) and lyophilized directly to give the product 3-(5-(difluoromethyl)-1,3,4-thiadiazol-2-yl)-8-(4-isobutyrylpiperazin-1-yl)-N-(1-methylcyclopropyl)imidazo[1,2-a]pyridine-6-sulfonamide (3.0 mg, 0.00553 mmol, 7.74% yield) as an off-white solid.

RT 0.619 min (Method 5); m/z 540.0 (M+H)⁺ (ESI⁺); ¹H NMR (CDCl₃, 400 MHz) 9.92 (s, 1H), 8.19 (s, 1H), 6.96-7.24 (m, 2H), 5.11 (s, 1H), 3.93 (s, 2H), 3.82 (s, 2H), 3.67 (s, 2H), 3.59 (s, 2H), 2.84-2.91 (m, 1H), 1.37 (s, 3H), 1.19 (d, J=6.8 Hz, 6H), 0.89-0.94 (m, 2H), 0.57-0.62 (m, 2H).

### Preparation of Intermediate 174.1

### Ethyl 6-(N-(tert-butoxycarbonyl)-N-(1-methylcyclopropyl)sulfamoyl)-8-chloroimidazo[1,2-a]pyridine-3-carboxylate

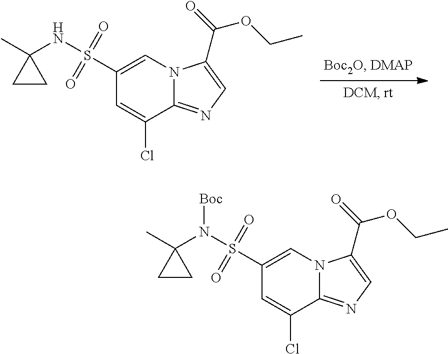

To a solution of ethyl 8-chloro-6-(N-(1-methylcyclopropyl)sulfamoyl)imidazo[1,2-a]pyridine-3-carboxylate (120 mg, 0.335 mmol) in DCM (1.5 mL) was added Boc₂O (95 mg, 0.436 mmol) and DMAP (8.2 mg, 0.0671 mmol). The reaction was stirred at 20° C. for 1 h, then diluted with H₂O (20 mL) and extracted with EtOAc (20 mL, 2×). The organic layer was washed with brine (20 mL, 2×), dried over anhydrous Na₂SO₄, filtered and concentrated under vacuum to give the product ethyl 6-(N-(tert-butoxycarbonyl)-N-(1-methylcyclopropyl)sulfamoyl)-8-chloroimidazo[1,2-a]pyridine-3-carboxylate (120 mg, 0.262 mmol, 78.14% yield) as a yellow solid.

RT 0.994 min (method 1); m/z 458.1 (M+H)⁺ (ESI⁺).

### Preparation of Intermediate 174.2

### tert-butyl ((8-chloro-3-(hydroxymethyl)imidazo[1,2-a]pyridin-6-yl)sulfonyl)(1-methylcyclopropyl)carbamate

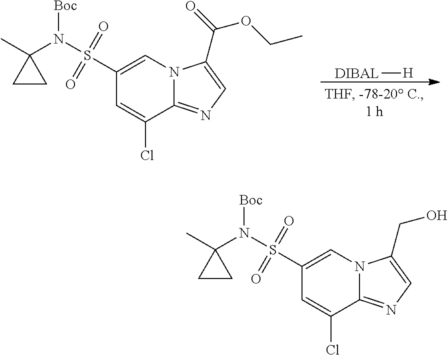

To a solution of ethyl 6-(N-(tert-butoxycarbonyl)-N-(1-methylcyclopropyl)sulfamoyl)-8-chloroimidazo[1,2-a]pyridine-3-carboxylate (130 mg, 0.284 mmol) in THE (1 mL) was added DIBAL-H in THE (1 M, 1.1 mL, 1.14 mmol) dropwise at −78° C. The reaction mixture was slowly warmed to 20° C. and stirred for 1 h. The reaction mixture was then diluted with saturated NH₄Cl (20 mL) and extracted with EtOAc (20 mL, 2×). The combined organic layer was washed with brine (20 mL, 2×), dried over anhydrous Na₂SO₄, filtered and concentrated under vacuum. The residue was purified by flash silica gel chromatography (ISCO®; 40 g SepaFlash® Silica Flash Column, Eluent of 0˜50% Ethyl Acetate/Petroleum ether @60 mL/min) to give the product tert-butyl ((8-chloro-3-(hydroxymethyl)imidazo[1,2-a]pyridin-6-yl)sulfonyl)(1-methylcyclopropyl)carbamate (80 mg, 0.192 mmol, 67.76% yield) as a yellow solid.

RT 0.592 min (Method 5); m/z 416.0 (M+H)⁺ (ESI⁺).

### Preparation of Intermediate 174.3

### tert-butyl ((8-chloro-3-(methoxymethyl)imidazo[1,2-a]pyridin-6-yl)sulfonyl)(1-methylcyclopropyl)carbamate

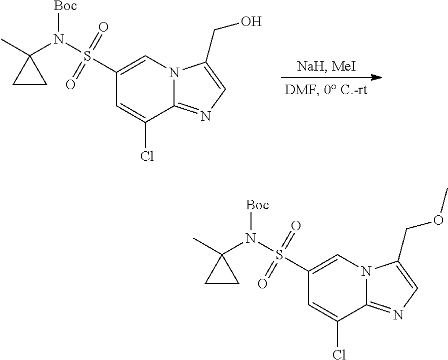

To a mixture of tert-butyl ((8-chloro-3-(hydroxymethyl)imidazo[1,2-a]pyridin-6-yl)sulfonyl)(1-methylcyclopropyl)carbamate (80 mg, 0.192 mmol) in DMF (0.5 mL) was added NaH (12 mg, 0.289 mmol, 60% purity) and MeI (0.036 mL, 0.577 mmol) at 0° C. The mixture was stirred at 20° C. for 1 hours, then diluted with NH₄Cl (aq. sat., 20 mL) and extracted with EtOAc (20 mL, 2×). The organic layer was washed with brine (20 mL, 2×), dried over anhydrous Na₂SO₄, filtered and concentrated under vacuum. The residue was purified by flash silica gel chromatography (ISCO®; 12 g SepaFlash® Silica Flash Column, Eluent of 0˜20% EtOAc/Petroleum ether gradient @60 mL/min) to give the product tert-butyl ((8-chloro-3-(methoxymethyl)imidazo[1,2-a]pyridin-6-yl)sulfonyl)(1-methylcyclopropyl)carbamate (50 mg, 0.116 mmol, 60.46% yield) as a yellow solid.

RT 0.636 min (Method 5); m/z 430.2 (M+H)⁺ (ESI⁺); ¹H NMR (DMSO-d₆, 400 MHz) 8.84 (d, J=1.6 Hz, 1H), 7.87 (s, 1H), 7.68 (d, J=1.6 Hz, 1H), 4.89 (s, 2H), 3.26 (s, 3H), 1.50 (s, 3H), 1.33 (s, 9H), 0.87-1.13 (m, 4H).

### Preparation of Intermediate 174.4

### tert-butyl ((8-(4-(dimethylcarbamoyl)piperazin-1-yl)-3-(methoxymethyl)imidazo[1,2-a]pyridin-6-yl)sulfonyl)(1-methylcyclopropyl)carbamate

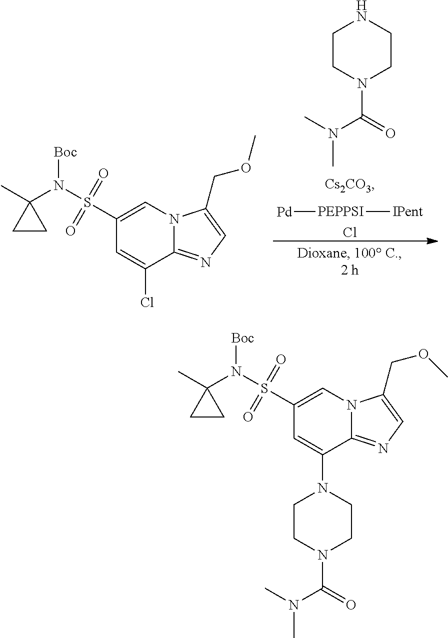

To a solution of tert-butyl ((8-chloro-3-(methoxymethyl)imidazo[1,2-a]pyridin-6-yl)sulfonyl)(1-methylcyclopropyl)carbamate (25 mg, 0.0582 mmol) in dioxane (0.5 mL) were added N,N-dimethylpiperazine-1-carboxamide (14 mg, 0.0872 mmol), Cs₂CO₃ (38 mg, 0.116 mmol) at room temperature. Then, to this solution was added Pd-PEPPSI-IPentCl o-picoline (2.8 mg, 0.00291 mmol) in a glove box. The mixture was stirred at 100° C. for 1 h outside of the glove box under Ar. The reaction mixture was cooled to room temperature, diluted with H₂O (20 mL) and extracted with EtOAc (20 mL, 2×). The combined organic layer was washed with brine (20 mL, 2×), dried over anhydrous Na₂SO₄, filtered and concentrated under vacuum to give the product tert-butyl ((8-(4-(dimethylcarbamoyl)piperazin-1-yl)-3-(methoxymethyl)imidazo[1,2-a]pyridin-6-yl)sulfonyl)(1-methylcyclopropyl)carbamate (30 mg, 0.0545 mmol, 93.69% yield).

RT 0.865 min (method 1); m/z 551.2 (M+H)⁺ (ESI⁺).

### Preparation of Example 174

### 4-(3-(methoxymethyl)-6-(N-(1-methylcyclopropyl)sulfamoyl)imidazo[1,2-a]pyridin-8-yl)-N,N-dimethylpiperazine-1-carboxamide

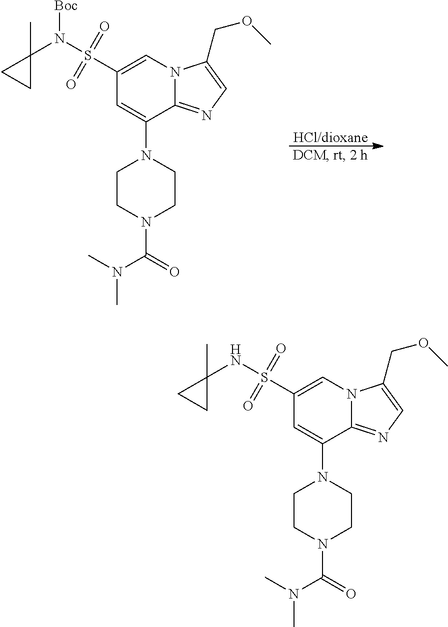 [SMILES: COCc1cnc2c(N3CCN(C(=O)N(C)C)CC3)cc(S(=O)(=O)N(C(=O)OC(C)(C)C)C3(C)CC3)cn12 ; "DCM, rt, 2 h" ; "HCl/dioxane" ; COCc1cnc2c(N3CCN(C(=O)N(C)C)CC3)cc(S(=O)(=O)NC3(C)CC3)cn12]

To a mixture of tert-butyl ((8-(4-(dimethylcarbamoyl)piperazin-1-yl)-3-(methoxymethyl)imidazo[1,2-a]pyridin-6-yl)sulfonyl)(1-methylcyclopropyl)carbamate (30 mg, 0.0545 mmol) in DCM (0.5 mL) was added HCl/dioxane (4 N, 0.50 mL). The reaction mixture was stirred at 20° C. for 1 h and then concentrated under vacuum. The residue was purified by preparative HPLC (column: Phenomenex Synergi C18 150*25 mm*10 μm; mobile phase: A: 0.225% formic acid in water, B: MeCN; B %: 22%-52%, 10 min) and lyophilized directly to give 4-(3-(methoxymethyl)-6-(N-(1-methylcyclopropyl)sulfamoyl)imidazo[1,2-a]pyridin-8-yl)-N,N-dimethylpiperazine-1-carboxamide (3.8 mg, 0.00835 mmol, 15.33% yield) as an off-white solid.

RT 0.506 min (Method 5); m/z 451.3 (M+H)⁺ (ESI⁺); ¹H NMR (CDCl₃, 400 MHz) 8.48 (d, J=1.2 Hz, 1H), 7.62 (s, 1H), 6.71 (s, 1H), 4.99 (s, 1H), 4.77 (s, 2H), 3.56-3.65 (m, 4H), 3.48-3.56 (m, 4H), 3.36 (s, 3H), 2.89 (s, 6H), 1.30 (s, 3H), 0.82-0.88 (m, 2H), 0.51-0.57 (m, 2H).

### Preparation of Example 175

### 3-(5-(difluoromethyl)-1,3,4-thiadiazol-2-yl)-8-(4-(N,S-dimethylsulfonimidoyl)piperidin-1-yl)-N-(1-methylcyclopropyl)imidazo[1,2-a]pyridine-6-sulfonamide

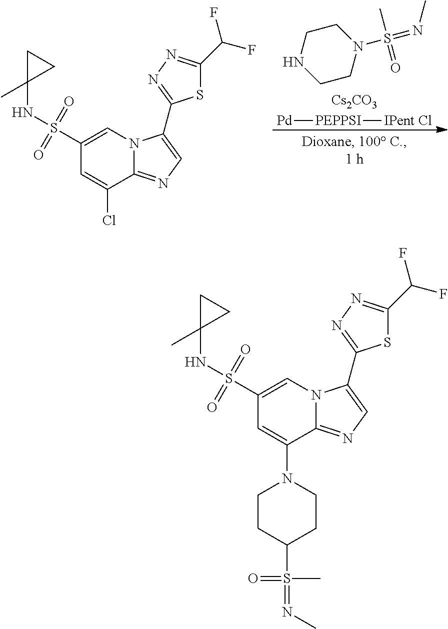

At room temperature, to a solution of 8-chloro-3-(5-(difluoromethyl)-1,3,4-thiadiazol-2-yl)-N-(1-methylcyclopropyl)imidazo[1,2-a]pyridine-6-sulfonamide (30 mg, 0.0715 mmol) in dioxane (0.5 mL) were added methyl(methylimino)(piperidin-4-yl)-A6-sulfanone (23 mg, 0.107 mmol, HCl salt), Cs₂CO₃ (70 mg, 0.214 mmol). Then, to this mixture was added Pd-PEPPSI-IPentCl o-picoline (3.5 mg, 0.00357 mmol) at room temperature in glove box. The mixture was stirred at 100° C. for 1 h outside of the glove box under Ar₂. The reaction mixture was purified by preparative TLC (PE/EtOAc=1/1) to give the product 3-(5-(difluoromethyl)-1,3,4-thiadiazol-2-yl)-8-(4-(N,S-dimethylsulfonimidoyl)piperidin-1-yl)-N-(1-methylcyclopropyl)imidazo[1,2-a]pyridine-6-sulfonamide (2.2 mg, 0.00367 mmol, 5.14% yield) as yellow gum.

RT 0.765 min (method 1); m/z 560.2 (M+H)⁺ (ESI⁺); ¹H NMR (CDCl₃, 400 MHz) 9.83 (d, J=1.6 Hz, 1H), 8.11 (s, 1H), 6.87-7.17 (m, 2H), 5.08 (s, 1H), 4.39-4.58 (m, 2H), 3.10-3.22 (m, 1H), 2.76-2.91 (m, 8H), 2.26-2.40 (m, 2H), 1.97-2.10 (m, 2H), 1.29 (s, 3H), 0.83-0.87 (m, 2H), 0.49-0.56 (m, 2H).

### Preparation of Example 176a

### tert-butyl 6-(3-(5-(difluoromethyl)-1,3,4-thiadiazol-2-yl)-6-(N-(1-methylcyclopropyl)sulfamoyl)imidazo[1,5-a]pyridin-8-yl)-2,6-diazaspiro[3.4]octane-2-carboxylate

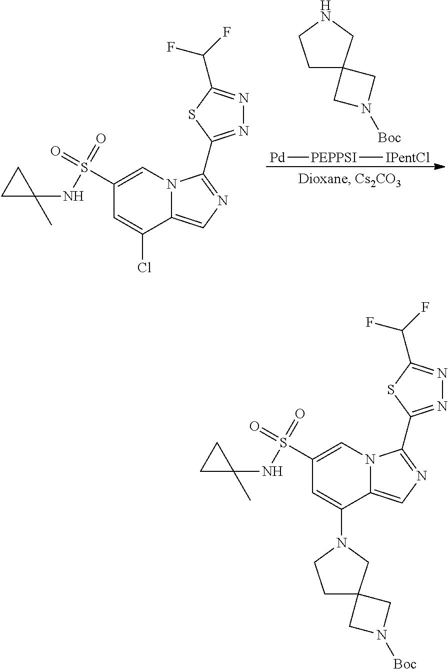

To a solution of tert-butyl 2,6-diazaspiro[3.4]octane-2-carboxylate (63 mg, 0.298 mmol) and 8-chloro-3-(5-(difluoromethyl)-1,3,4-thiadiazol-2-yl)-N-(1-methylcyclopropyl)imidazo[1,5-a]pyridine-6-sulfonamide (50 mg, 0.119 mmol) in dioxane (1.5 mL) was added Cs₂CO₃ (78 mg, 0.238 mmol). The mixture was degassed with N₂ (3×), then Pd-PEPPSI-IPentCl o-picoline (12 mg, 0.0119 mmol) was added. The mixture was stirred at 90° C. for 2 h under N₂, and purified by preparative TLC (petroleum ether/EtOAc=2/1) to give the product tert-butyl 6-(3-(5-(difluoromethyl)-1,3,4-thiadiazol-2-yl)-6-(N-(1-methylcyclopropyl)sulfamoyl)imidazo[1,5-a]pyridin-8-yl)-2,6-diazaspiro[3.4]octane-2-carboxylate (20 mg, 0.0336 mmol, 28.19% yield) as a yellow solid.

RT 0.524 min (method 1); m/z 596.4 (M+H)⁺ (ESI⁺); ¹H NMR (DMSO-d₆, 400 MHz) 9.38 (s, 1H), 8.33 (s, 1H), 8.18 (s, 1H), 7.49-7.82 (m, 1H), 6.13 (s, 1H), 3.91-3.98 (m, 2H), 3.82-3.90 (m, 4H), 3.68 (t, J=6.8 Hz, 2H), 2.22-2.30 (m, 2H), 1.39 (s, 9H), 1.19 (s, 3H), 0.73 (m, 2H), 0.39-0.50 (m, 2H).

### Preparation of Example 176

### 3-(5-(difluoromethyl)-1,3,4-thiadiazol-2-yl)-N-(1-methylcyclopropyl)-8-(2,6-diazaspiro[3.4]octan-6-yl)imidazo[1,5-a]pyridine-6-sulfonamide

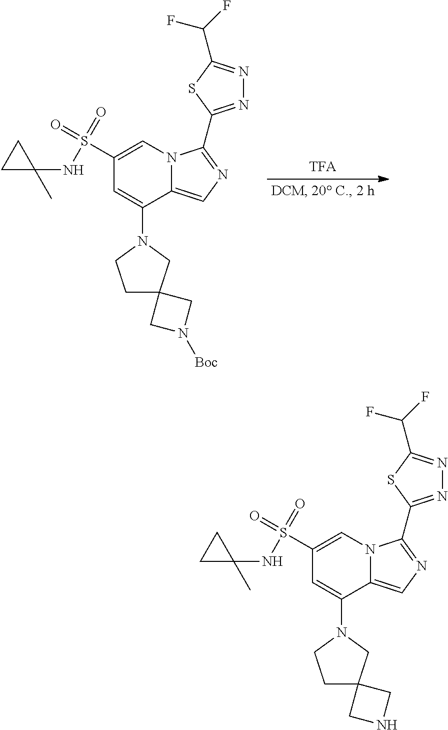 [SMILES: CC1(NS(=O)(=O)c2cc(N3CCC4(CN(C(=O)OC(C)(C)C)C4)C3)c3cnc(-c4nnc(C(F)F)s4)n3c2)CC1 ; CC1(NS(=O)(=O)c2cc(N3CCC4(CNC4)C3)c3cnc(-c4nnc(C(F)F)s4)n3c2)CC1]

To a solution of tert-butyl 6-(3-(5-(difluoromethyl)-1,3,4-thiadiazol-2-yl)-6-(N-(1-methylcyclopropyl)sulfamoyl)imidazo[1,5-a]pyridin-8-yl)-2,6-diazaspiro[3.4]octane-2-carboxylate (20 mg, 0.0336 mmol) in DCM (1 mL) was added TFA (0.67 mL, 9.00 mmol). The mixture was stirred at 20° C. for 1 h and then, concentrated under reduced pressure. The residue was triturated with MTBE at 20° C. for 1 min. The resulting suspension was standing for a moment, then the supernatant was discarded (3×). The solid was collected and dried under vacuum. The residue was dissolved in water (10 mL) and directly lyophilized to give the product 3-(5-(difluoromethyl)-1,3,4-thiadiazol-2-yl)-N-(1-methylcyclopropyl)-8-(2,6-diazaspiro[3.4]octan-6-yl)imidazo[1,5-a]pyridine-6-sulfonamide (4.8 mg, 0.00713 mmol, 21.25% yield, TFA salt) as yellow solid.

RT 0.362 min (method 1); m/z 496.2 (M+H)⁺ (ESI⁺); ¹H NMR (DMSO-d₆, 400 MHz) 9.41 (s, 1H), 8.50-8.83 (m, 2H), 8.38 (s, 1H), 8.15 (s, 1H), 7.51-7.84 (m, 1H), 6.13 (s, 1H), 4.09-4.17 (m, 2H), 3.91-4.02 (m, 4H), 3.67-3.76 (m, 2H), 2.34 (m, 2H), 1.20 (s, 3H), 0.70-0.77 (m, 2H), 0.43-0.49 (m, 2H).

### Preparation of Example 177a

### tert-butyl 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)piperazine-1-carboxylate

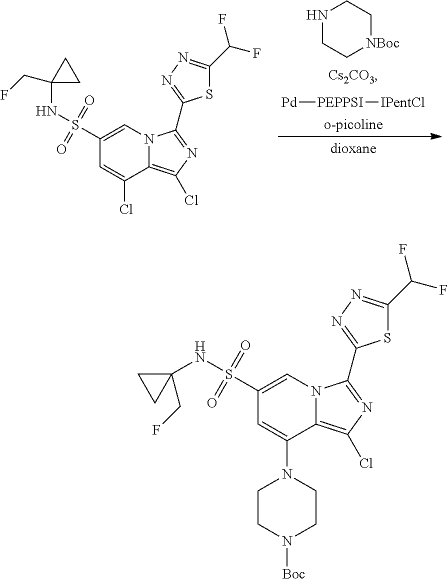

To a solution of 1,8-dichloro-3-(5-(difluoromethyl)-1,3,4-thiadiazol-2-yl)-N-(1-(fluoromethyl)cyclopropyl)imidazo[1,5-a]pyridine-6-sulfonamide (25 mg, 52.93 μmol) and tert-butyl piperazine-1-carboxylate (23.58 mg, 105.87 μmol, HCl salt) in dioxane (1.5 mL) was added Pd-PEPPSI-IPentCl o-picoline (2.57 mg, 2.65 μmol) and Cs₂CO₃ (51.74 mg, 158.80 μmol) and the mixture was degassed with N₂ (3×). Then, the mixture was stirred at 90° C. for 5 h, cooled to room temperature, filtered and the filtrate was concentrated under vacuum. The residue was purified by preparative TLC (Petroleum ether/EtOAc=2/1) to give the product tert-butyl 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)piperazine-1-carboxylate (20 mg, 28.94 μmol, 54.66% yield, 90% purity) as a yellow solid.

RT 0.549 min (Method 5); m/z 622.3 (M+H)⁺ (ESI⁺); ¹H NMR (DMSO-d₆, 400 MHz) 9.67 (s, 1H), 8.94 (s, 1H), 7.76 (t, J=52.8 Hz, 1H), 6.92 (s, 1H), 4.29 (d, J=48.4 Hz, 2H), 3.67-3.65 (m, 4H), 3.15-3.12 (m, 4H), 1.51 (s, 9H), 0.96-0.90 (m, 2H), 0.86-0.83 (m, 2H).

### Preparation of Example 177b

### 1-chloro-3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide formate

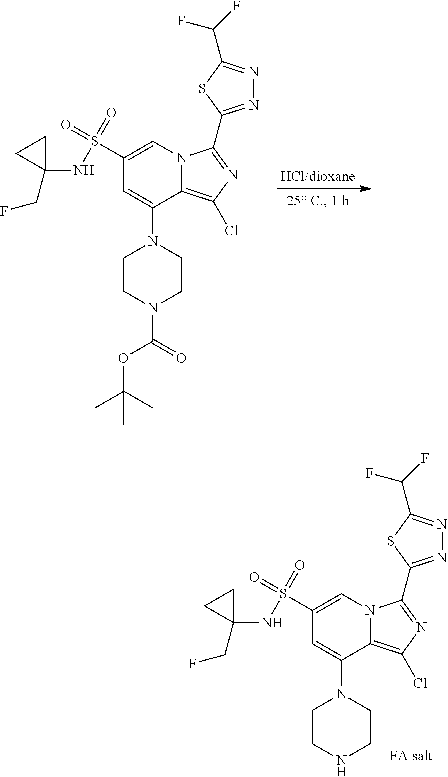 [SMILES: CC(C)(C)OC(=O)N1CCN(c2cc(S(=O)(=O)NC3(CF)CC3)cn3c(-c4nnc(C(F)F)s4)nc(Cl)c23)CC1 ; O=S(=O)(NC1(CF)CC1)c1cc(N2CCNCC2)c2c(Cl)nc(-c3nnc(C(F)F)s3)n2c1]

A mixture of tert-butyl 4-(1-chloro-3-(5-(difluoromethyl)-1,3,4-thiadiazol-2-yl)-6-(N-(1-(fluoromethyl)cyclopropyl)sulfamoyl)imidazo[1,5-a]308yridine-8-yl)piperazine-1-carboxylate (30 mg, 48.23 μmol) in HCl/dioxane (1 mL, 4.00 mmol, 4N) was stirred at 25° C. for 1 h and concentrated under vacuum to give a crude product (30 mg). 15 mg were purified by preparative HPLC (column: Phenomenex luna C18 150*25 mm*10 μm; mobile phase: A: 0.225% formic acid in water, B: MeCN; B %: 15%-45%, 10 min) and lyophilized to give the product 1-chloro-3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide formate (3.21 mg, 5.43 μmol, 25.51% yield, 96% purity, FA salt) as a yellow solid.

RT 0.367 min (Method 3); m/z 522.3 (M+H)⁺ (ESI⁺); ¹H NMR (DMSO-d₆, 400 MHz): 9.59 (s, 1H), 8.91 (br, 1H), 8.23 (d, J=1.6 Hz, 1H), 7.70 (t, J=53.2, 1H), 6.81 (s, 1H), 4.23 (t, J=48.4, 2H), 3.75-3.02 (m, 4H), 3.02-2.96 (m, 4H), 0.90-0.85 (m, 2H), 0.81-0.76 (m, 2H).

### Preparation of Example 177

### 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N-methylpiperazine-1-carboxamide

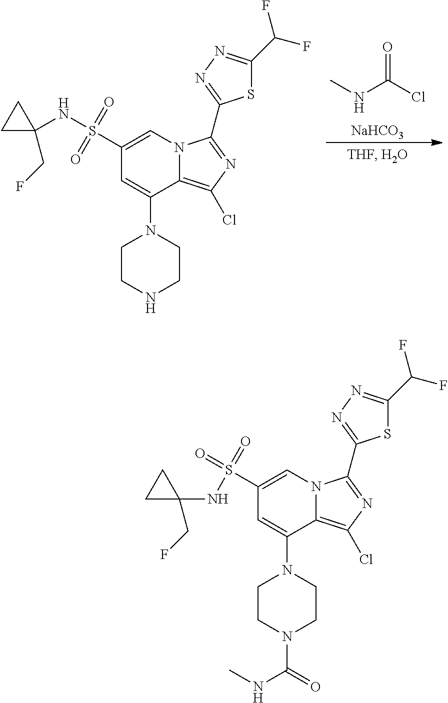

To a mixture of 1-chloro-3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide (15 mg, 28.74 μmol) in THE (0.2 mL) and NaHCO₃ (aq., sat., 1.5 mL) was added methylcarbamic chloride (5.37 mg, 57.47 μmol) at 0° C. The mixture was stirred at 20° C. for 30 min, then diluted with DCM (10 mL), washed with water (5 mL; 3×) and separated. The combined organic layer was dried over anhydrous Na₂SO₄, filtered, and the filtrate was concentrated undervacuum. The residue was purified by preparative TLC (petroleum ether:EtOAc=1/1) and preparative HPLC (column: Phenomenex luna C18 150*25 mm*10 μm; mobile phase: A: 0.225% formic acid in water, B: MeCN; B %: 37%-67%, 10 min) to give an impure product which was further purified by trituration with MeOH (0.5 mL, 20° C., 5 min). The solid was collected by filtration and dissolved in MeCN (1 mL). To the solution was added water (15 mL) and it lyophilized to give the product 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-N-methylpiperazine-1-carboxamide (6.04 mg, 10.36 μmol, 36.05% yield, 99.3% purity) as a yellow solid.

RT 0.452 min (Method 5); m/z 579.2 (M+H)⁺ (ESI⁺); ¹H NMR (DMSO-d₆, 400 MHz) 9.60 (s, 1H), 8.88 (s, 1H), 7.69 (t, J=52.8 Hz, 1H), 6.84 (s, 1H), 6.59-6.57 (m, 1H), 4.22 (d, J=48.4 Hz, 2H), 3.62-3.52 (m, 4H), 3.14-2.99 (m, 4H), 2.61 (d, J=4.4 Hz, 3H), 0.90-0.84 (m, 2H), 0.81-0.75 (m, 2H).

### Preparation of Example 178a

### (S)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate

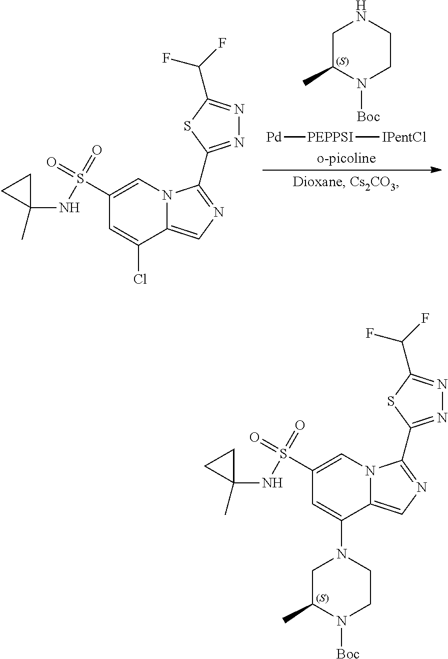

A mixture of 8-chloro-3-(5-(difluoromethyl)-1,3,4-thiadiazol-2-yl)-N-(1-methylcyclopropyl)imidazo [1,5-a]pyridine-6-sulfonamide (100 mg, 0.238 mmol), tert-butyl (S)-2-methylpiperazine-1-carboxylate (191 mg, 0.953 mmol), Pd-PEPPSI-IPentCl o-picoline (23 mg, 0.0238 mmol), Cs₂CO₃ (233 mg, 0.715 mmol) in dioxane (3 mL) was degassed and purged with N₂ (3×). The mixture was stirred at 95° C. for 4 h under N₂ atmosphere, cooled to room temperature and concentrated under reduce pressure. The residue was purified by preparative TLC (petroleum ether:EtOAc=2/1) to give the product (S)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (65 mg, 0.105 mmol, 43.95% yield) as a yellow solid.

RT 0.537 min (Method 5); m/z 528.2 (M−56+H)⁺ (ESI⁺); ¹H NMR (CDCl₃, 400 MHz): 9.87 (s, 1H), 7.73 (s, 1H), 7.09 (t, J=54 Hz, 1H), 6.65 (s, 1H), 5.08 (s, 1H), 4.47-4.44 (m, 1H), 4.07 (d, J=13.2 Hz, 1H), 3.62-3.34 (m, 2H), 3.37 (t, J=12.8 Hz, 1H), 3.08-2.94 (m, 2H), 1.52 (s, 9H), 1.47 (d, J=6.8 Hz, 3H), 1.39 (s, 3H), 0.94 (s, 2H), 0.61 (s, 2H).

### Preparation of Example 178

### (S)-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate

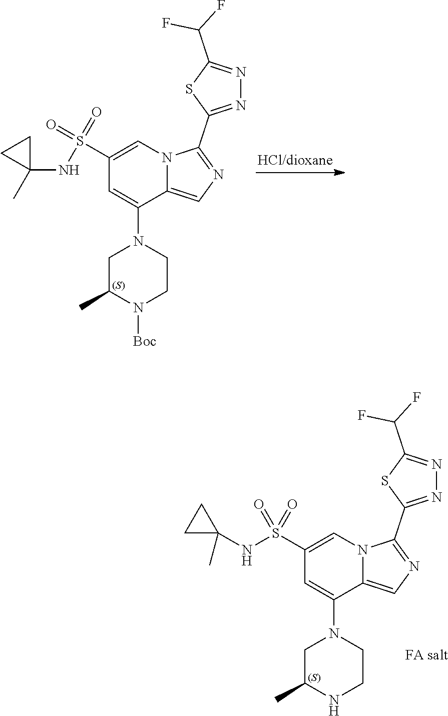

A solution of (S)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (50 mg, 0.0857 mmol) in HCl/dioxane (2.0 mL, 0.0857 mmol, 4 N) was stirred at 20° C. for 1 h. After concentration under reduce pressure, the crude product was triturated with MeOH (0.5 mL) at 20° C. for 5 min. The solid was collected by filtration and it was further purified by preparative HPLC (column: Phenomenex Luna C18 150*25 mm*5 μm; mobile phase: A: 0.225% formic acid in water, B: MeCN; B %: 18%-48%, 10 min) and lyophilized directly to give the product (S)-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate (35 mg, 0.0661 mmol, 77.15% yield, FA salt) as a yellow solid.

RT 0.358 min (Method 3); m/z 484.3 (M+H)⁺ (ESI⁺); ¹H NMR (CDCl₃, 400 MHz): 9.87 (s, 1H), 8.31 (s, 1H), 7.70 (s, 1H), 7.09 (t, J=54 Hz, 1H), 6.70 (s, 1H), 5.44-5.38 (m, 1H), 3.66 (t, J=15.2 Hz, 2H), 3.63-3.25 (m, 3H), 3.12 (t, J=11.6 Hz, 1H), 2.83 (t, J=11.6 Hz, 1H), 1.38 (s, 3H), 1.32 (d, J=6.4 Hz, 3H), 0.95-0.92 (m, 2H), 0.61-0.58 (m, 2H).

### Preparation of Example 179

### (S)-3-(5-(difluoromethyl)-1,3,4-thiadiazol-2-yl)-8-(4-isobutyryl-3-methylpiperazin-1-yl)-N-(1-methylcyclopropyl)imidazo[1,5-a]pyridine-6-sulfonamide

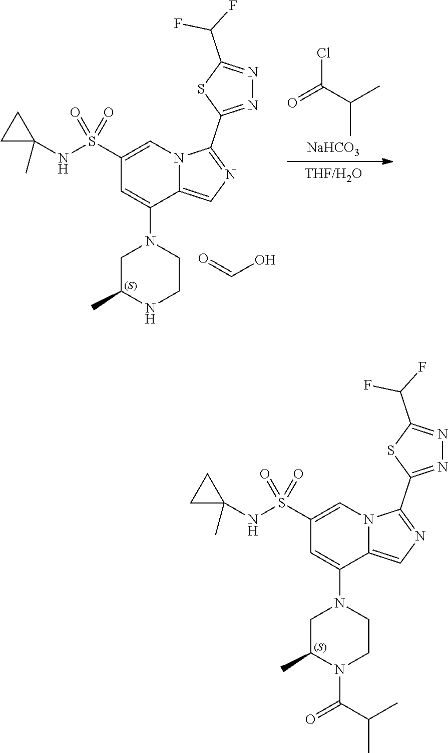

To a solution of (S)-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate (20 mg, 0.0378 mmol, FA salt) in THE (0.5 mL) and water (0.5 mL) were added NaHCO₃ (25 mg, 0.302 mmol) and 2-methylpropanoyl chloride (8.0 mg, 0.0755 mmol). The mixture was stirred at 0° C. for 1 h then diluted with water (20 mL) and extracted with EtOAc (50 mL, 3×). The combined organic layer was washed with brine (25 mL, 2×), dried over anhydrous Na₂SO₄, filtered and concentrated under vacuum. The residue was purified by preparative HPLC (column: Phenomenex luna C18 150*25 mm*10 μm; mobile phase: A: 0.225% formic acid in water, B: MeCN; B %: 44%-74%, 10 min) and lyophilized directly to give the product (S)-3-(5-(difluoromethyl)-1,3,4-thiadiazol-2-yl)-8-(4-isobutyryl-3-methylpiperazin-1-yl)-N-(1-methylcyclopropyl)imidazo[1,5-a]pyridine-6-sulfonamide (3.1 mg, 0.00558 mmol, 14.77% yield) as a yellow solid.

RT 0.487 min (Method 3); m/z 554.4 (M+H)⁺ (ESI⁺); ¹H NMR (CDCl₃, 400 MHz): 9.89 (s, 1H), 7.74 (s, 1H), 7.09 (t, J=54 Hz, 1H), 6.66 (s, 1H), 5.11 (s, 1H), 5.06-486 (m, 0.5H), 4.78-4.56 (m, 0.5H), 4.47-4.21 (m, 0.5H), 4.07-3.83 (m, 0.5H), 3.62-3.75 (m, 2.5H), 3.16-3.32 (m, 0.5H), 2.90-3.11 (m, 2H), 2.77-2.88 (m, 1H), 1.49 (br d, J=7.60 Hz, 2H), 1.40 (s, 3H), 1.21 (br d, J=6.64 Hz, 7H), 0.94 (br s, 2H), 0.61 (s, 2H).

### Preparation of Example 180a

### (S)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-ethylpiperazine-1-carboxylate

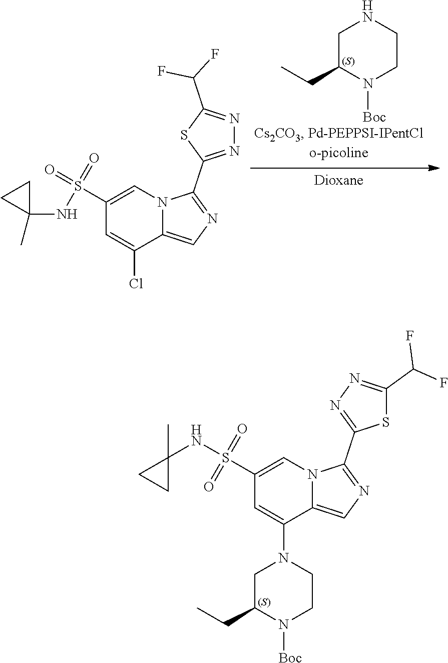

To a mixture of 8-chloro-3-(5-(difluoromethyl)-1,3,4-thiadiazol-2-yl)-N-(1-methylcyclopropyl)imidazo[1,5-a]pyridine-6-sulfonamide (30 mg, 0.0715 mmol) in dioxane (0.5 mL) was added (S)-tert-butyl 2-ethylpiperazine-1-carboxylate (31 mg, 0.143 mmol), Cs₂CO₃ (70 mg, 0.214 mmol) and Pd-PEPPSI-IPentCl o-picoline (7.0 mg, 0.00715 mmol). The reaction mixture was degassed with N₂ (3×), stirred at 98° C. for 1 h and filtered. The filtrate was concentrated under reduce pressure to give a residue, which was purified by preparative TLC (Petroleum ether:Ethyl acetate=1:1) to give the product (S)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-ethylpiperazine-1-carboxylate (25 mg, 0.0387 mmol, 54.14% yield, 92.4% purity) as a yellow solid.

RT 0.597 min (Method 4); m/z 620.2 (M+Na)⁺ (ESI⁺); ¹H NMR (CDCl₃, 400 MHz): 9.87 (s, 1H), 7.72 (s, 1H), 7.08 (t, J=54.0 Hz, 1H), 6.65 (s, 1H), 5.09 (s, 1H), 4.30-4.14 (m, 2H), 3.70-3.49 (m, 2H), 3.38-3.19 (m, 1H), 3.11-2.88 (m, 2H), 2.15-2.07 (m, 1H), 1.79-1.71 (m, 1H), 1.51 (s, 9H), 1.39 (s, 3H), 1.00 (t, J=7.2 Hz, 3H), 0.96-0.90 (m, 2H), 0.64-0.57 (m, 2H).

### Preparation of Example 180

### (S)-3-(5-(difluoromethyl)-1,3,4-thiadiazol-2-yl)-8-(3-ethylpiperazin-1-yl)-N-(1-methylcyclopropyl)imidazo[1,5-a]pyridine-6-sulfonamide formate

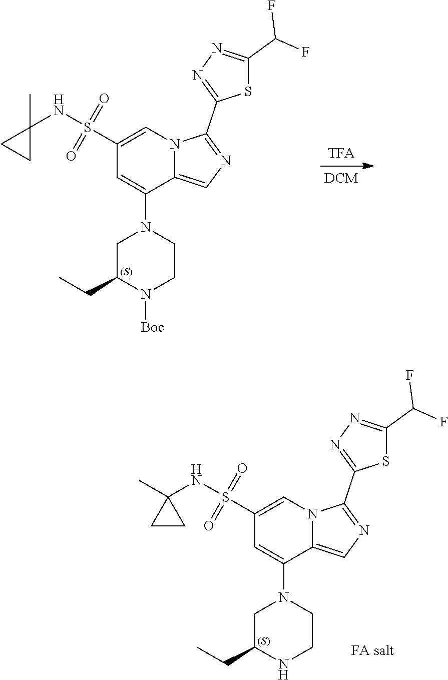

A solution of (S)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-ethylpiperazine-1-carboxylate (25 mg, 0.0402 mmol) in DCM (0.5 mL) and TFA (0.1 mL) was stirred at 25° C. for 1 h. The mixture was concentrated undervacuum to give a residue, which was purified by preparative HPLC (column: Phenomenex luna C18 150*25 mm*10 um; mobile phase: A: 0.225% formic in water, B: MeCN; B %: 12%-42%, 15 min) and lyophilized directly to give the product (S)-3-(5-(difluoromethyl)-1,3,4-thiadiazol-2-yl)-8-(3-ethylpiperazin-1-yl)-N-(1-methylcyclopropyl)imidazo[1,5-a]pyridine-6-sulfonamide formate (9.4 mg, 0.0168 mmol, 41.79% yield, 97.8% purity, FA salt) as a yellow solid.

RT 0.366 min (method 4); m/z 498.0 (M+H)⁺ (ESI⁺); ¹H NMR (DMSO-d₆, 400 MHz): 9.58 (s, 1H), 8.43 (br, 1H), 8.23 (s, 1H), 7.96 (s, 1H), 7.67 (t, J=53.2 Hz, 1H), 6.67 (s, 1H), 3.63-3.58 (m, 2H), 3.11-3.04 (m, 1H), 3.02-2.94 (m, 1H), 2.89-2.78 (m, 2H), 2.54-2.56 (m, 1H), 1.46-1.42 (m, 2H), 1.15 (s, 3H), 0.96 (t, J=7.6 Hz, 3H), 0.77-0.67 (m, 2H), 0.47-0.42 (m, 2H).

### Preparation of Example 181a

### (R)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-ethylpiperazine-1-carboxylate

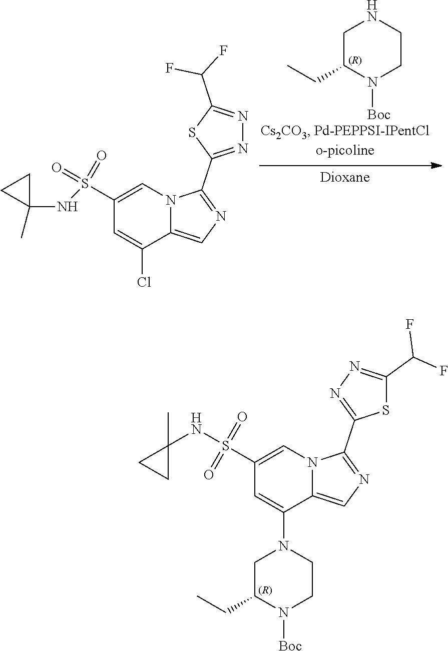

To a solution of 8-chloro-3-(5-(difluoromethyl)-1,3,4-thiadiazol-2-yl)-N-(1-methylcyclopropyl)imidazo[1,5-a]pyridine-6-sulfonamide (30 mg, 0.0715 mmol) in dioxane (0.5 mL) were added (R)-tert-butyl 2-ethylpiperazine-1-carboxylate (31 mg, 0.143 mmol), Cs₂CO₃ (70 mg, 0.214 mmol) and Pd-PEPPSI-IPentCl o-picoline (7.0 mg, 0.00715 mmol). The reaction mixture was degassed with N₂ (3×), then stirred at 98° C. for 1 h and filtered. The filtrate was concentrated under reduce pressure to give a residue which was purified by preparative TLC (Petroleum ether:Ethyl acetate=1:1) to give the product (R)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-ethylpiperazine-1-carboxylate (20 mg, 0.0333 mmol, 46.56% yield, 99.4% purity) as a yellow solid.

RT 0.591 min (method 4), m/z 620.1 (M+Na)⁺ (ESI⁺); ¹H NMR (CDCl₃, 400 MHz) 9.87 (s, 1H), 7.72 (s, 1H), 7.08 (t, J=53.6 Hz, 1H), 6.64 (s, 1H), 5.09 (s, 1H), 4.28-4.14 (m, 2H), 3.70-3.49 (m, 2H), 3.38-3.19 (m, 1H), 3.11-2.88 (m, 2H), 2.15-2.07 (m, 1H), 1.82-1.71 (m, 1H), 1.51 (s, 9H), 1.39 (s, 3H), 1.00 (t, J=7.6 Hz, 3H), 0.96-0.90 (m, 2H), 0.64-0.58 (m, 2H).

### Preparation of Example 181

### (R)-3-(5-(difluoromethyl)-1,3,4-thiadiazol-2-yl)-8-(3-ethylpiperazin-1-yl)-N-(1-methylcyclopropyl)imidazo[1,5-a]pyridine-6-sulfonamide formate

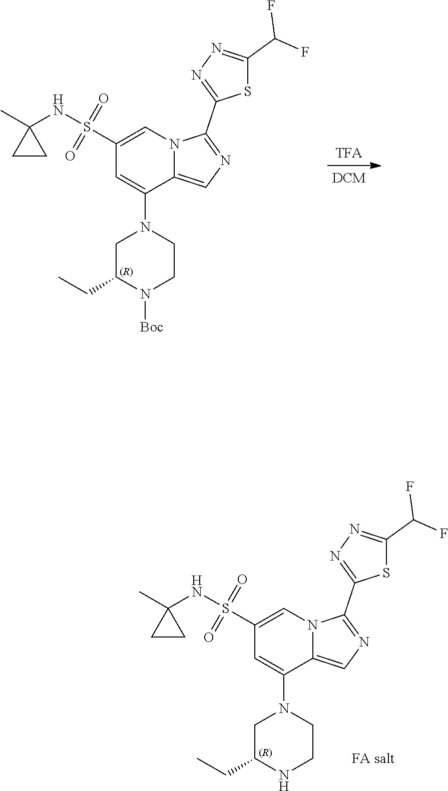

A mixture of (R)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-ethylpiperazine-1-carboxylate (20 mg, 0.0335 mmol) in DCM (0.2 mL) and TFA (0.04 mL) was stirred at 25° C. for 1 h. The mixture was concentrated under vacuum to give a residue which was purified by preparative HPLC (column: Phenomenex luna C18 150*25 mm*10 um; mobile phase: A: 0.225% formic in water, B: MeCN; B %: 13%-43%, 10 min) and lyophilized directly to give the product (R)-3-(5-(difluoromethyl)-1,3,4-thiadiazol-2-yl)-8-(3-ethylpiperazin-1-yl)-N-(1-methylcyclopropyl)imidazo[1,5-a]pyridine-6-sulfonamide formate (7.6 mg, 0.0133 mmol, 39.57% yield, 95.6% purity, FA salt) as a yellow solid.

RT 0.363 min (method 4); m/z 498.1 (M+H)⁺ (ESI⁺); ¹H NMR (DMSO-d₆, 400 MHz): 9.57 (s, 1H), 8.43 (br, 1H), 8.19 (s, 1H), 7.97 (s, 1H), 7.67 (t, J=53.2 Hz, 1H), 6.67 (d, J=0.8 Hz, 1H), 3.63-3.60 (m, 2H), 3.11-3.04 (m, 1H), 3.02-2.94 (m, 1H), 2.89-2.78 (m, 2H), 2.56 (br d, J=10.8 Hz, 1H), 1.51-1.42 (m, 2H), 1.14 (s, 3H), 0.96 (t, J=7.6 Hz, 3H), 0.75-0.69 (m, 2H), 0.47-0.42 (m, 2H).

### Preparation of Intermediate 182.1

### tert-butyl 4-((2-(dimethylamino)ethyl)(methyl)carbamoyl)piperazine-1-carboxylate

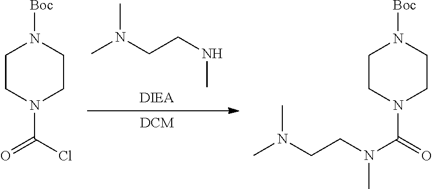

To a solution of N¹,N¹,N²-trimethylethane-1,2-diamine (80 mg, 0.783 mmol) and DIEA (0.27 mL, 1.57 mmol) in DCM (4 mL) was added a solution of tert-butyl 4-(chlorocarbonyl)piperazine-1-carboxylate (234 mg, 0.94 mmol) in DCM (1 mL) at 0° C. The reaction mixture was stirred at 20° C. for 15 min, then concentrated under vacuum. The resulting residue was diluted with saturated NaHCO₃ (20 mL) and extracted with EtOAc (20 mL, 2×). The combined organic layer was washed with brine (20 mL, 2×), dried over anhydrous Na₂SO₄, filtered and concentrated under vacuum to give tert-butyl 4-((2-(dimethylamino)ethyl)(methyl)carbamoyl)piperazine-1-carboxylate (200 mg, 0.496 mmol, 63.37% yield) as a white solid.

¹H NMR (CDCl₃, 400 MHz) 3.40-3.45 (m, 4H), 3.30-3.37 (m, 2H), 3.13-3.21 (m, 4H), 2.89 (s, 3H), 2.49-2.57 (m, 2H), 2.29 (s, 6H), 1.46 (s, 9H).

### Preparation of Intermediate 182.2

### N-(2-(dimethylamino)ethyl)-N-methylpiperazine-1-carboxamide hydrochloride

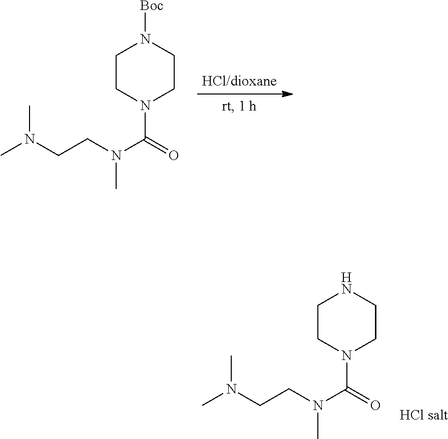 [SMILES: CN(C)CCN(C)C(=O)N1CCN(C(=O)OC(C)(C)C)CC1 ; CN(C)CCN(C)C(=O)N1CCNCC1]

A mixture of tert-butyl 4-((2-(dimethylamino)ethyl)(methyl)carbamoyl)piperazine-1-carboxylate (80 mg, 0.254 mmol) in HCl/dioxane (1.5 mL, 0.254 mmol) was stirred at 25° C. for 2 h. The resulting mixture was concentrated under vacuum. The residue was triturated with MTBE at 20° C. for 1 min. The resulting suspension was standing for a moment and then, the supernatant was discarded (3×). The solid was collected and dried under reduced pressure to give N-(2-(dimethylamino)ethyl)-N-methylpiperazine-1-carboxamideN-(2-(dimethylamino)ethyl)-N-methylpiperazine-1-carboxamide hydrochloride (46 mg, 0.165 mmol, 64.89% yield, HCl salt) as a colorless oil.

### Preparation of Example 182

### 4-(3-(5-(difluoromethyl)-1,3,4-thiadiazol-2-yl)-6-(N-(1-methylcyclopropyl)sulfamoyl)imidazo[1,5-a]pyridin-8-yl)-N-(2-(dimethylamino)ethyl)-N-methylpiperazine-1-carboxamide

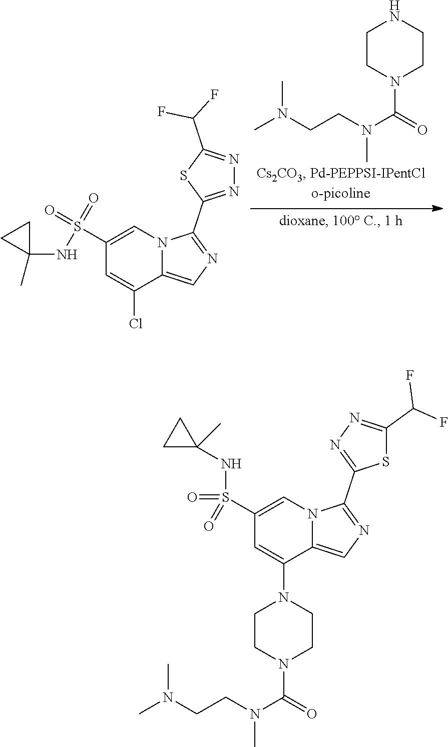

To a mixture of 8-chloro-3-(5-(difluoromethyl)-1,3,4-thiadiazol-2-yl)-N-(1 methylcyclopropyl)imidazo[1,5-a]pyridine-6-sulfonamide (30 mg, 0.0715 mmol) in 1,4-Dioxane (1 mL) were added N-(2-(dimethylamino)ethyl)-N-methylpiperazine-1-carboxamide (36 mg, 0.143 mmol, HCl salt), Cs₂CO₃ (47 mg, 0.143 mmol) and Pd-PEPPSI-IPentCl o-picoline (7.0 mg, 0.00715 mmol). The mixture was degassed, purged with N₂ for (3×) and stirred at 100° C. for 2 h under N₂ atmosphere. The reaction mixture was filtered and the filtrate was concentrated under vacuum. The residue was purified by preparative HPLC (column: Phenomenex luna C18 150*25 mm*10 um; mobile phase: A: 0.225% formic acid in water, B: MeCN; B %: 15%-45%, 10 min) and lyophilized directly to give the product 4-(3-(5-(difluoromethyl)-1,3,4-thiadiazol-2-yl)-6-(N-(1-methylcyclopropyl)sulfamoyl)imidazo[1,5-a]pyridin-8-yl)-N-(2-(dimethylamino)ethyl)-N-methylpiperazine-1-carboxamide (5.8 mg, 0.00956 mmol, 13.38% yield) as a yellow solid.

RT 0.361 min (method 4); m/z 598.0 (M+H)⁺ (ESI⁺); ¹H NMR (CDCl₃, 400 MHz) 9.88 (s, 1H), 7.71 (s, 1H), 7.08 (t, J=53.6 Hz, 1H), 6.67 (s, 1H), 5.27 (s, 1H), 3.51-3.55 (m, 4H), 3.44 (t, J=6.8 Hz, 2H), 3.33-3.37 (m, 4H), 2.98 (s, 3H), 2.68 (t, J=7.2 Hz, 2H), 2.41 (s, 6H), 1.38 (s, 3H), 0.91-0.95 (m, 2H), 0.57-0.62 (m, 2H)

### Preparation of Example 183a

### tert-butyl 3-(3-(5-(difluoromethyl)-1,3,4-thiadiazol-2-yl)-6-(N-(1-methylcyclopropyl)sulfamoyl)imidazo[1,5-a]pyridin-8-yl)-3,6-diazabicyclo[3.2.1]octane-6-carboxylate

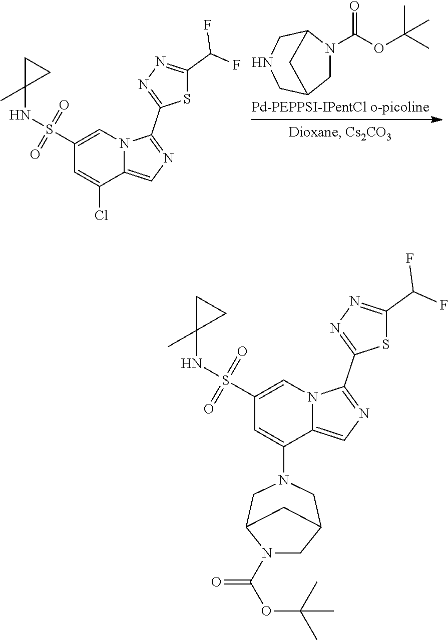

A mixture of 8-chloro-3-(5-(difluoromethyl)-1,3,4-thiadiazol-2-yl)-N-(1-methylcyclopropyl)imidazo [1,5-a]pyridine-6-sulfonamide (50 mg, 0.119 mmol), tert-butyl 3,6-diazabicyclo[3.2.1]octane-6-carboxylate (51 mg, 0.238 mmol), Pd-PEPPSI-IPentCl o-picoline (12 mg, 0.0119 mmol) and Cs₂CO₃ (116 mg, 0.357 mmol) in dioxane (1 mL) was degassed, purged with N₂ (3×) and stirred at 95° C. for 4 h under N₂ atmosphere. Then, the reaction mixture was cooled to room temperature and concentrated undervacuum. The residue was purified by preparative TLC (petroleum ether/EtOAc=1/1) to give the product tert-butyl 3-(3-(5-(difluoromethyl)-1,3,4-thiadiazol-2-yl)-6-(N-(1-methylcyclopropyl)sulfamoyl)imidazo[1,5-a]pyridin-8-yl)-3,6-diazabicyclo[3.2.1]octane-6-carboxylate (13 mg, 0.0199 mmol, 16.68% yield) as a yellow solid.

RT 0.516 min (Method 3); m/z 596.1 (M+H)⁺ (ESI⁺);

### Preparation of Example 183

### 8-(3,6-diazabicyclo[3.2.1]octan-3-yl)-3-(5-(difluoromethyl)-1,3,4-thiadiazol-2-yl)-N-(1-methylcyclopropyl)imidazo[1,5-a]pyridine-6-sulfonamide formate

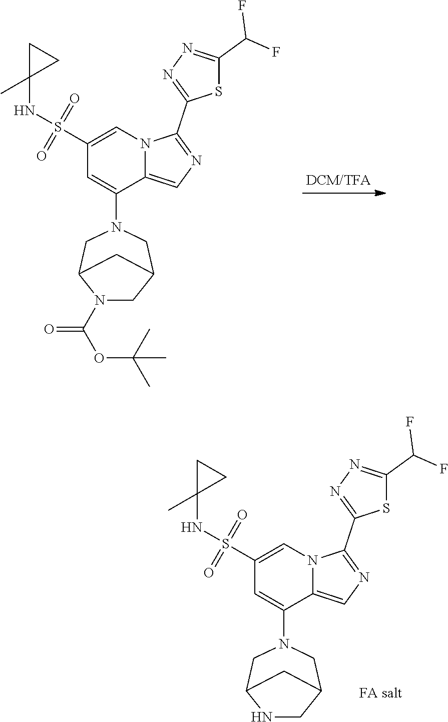 [SMILES: CC1(NS(=O)(=O)c2cc(N3CC4CC(C3)N(C(=O)OC(C)(C)C)C4)c3cnc(-c4nnc(C(F)F)s4)n3c2)CC1 ; CC1(NS(=O)(=O)c2cc(N3CC4CNC(C4)C3)c3cnc(-c4nnc(C(F)F)s4)n3c2)CC1]

At room temperature, to a solution of tert-butyl 3-(3-(5-(difluoromethyl)-1,3,4-thiadiazol-2-yl)-6-(N-(1-methylcyclopropyl)sulfamoyl)imidazo[1,5-a]pyridin-8-yl)-3,6-diazabicyclo[3.2.1]octane-6-carboxylate (10 mg, 0.0168 mmol) in DCM (0.5 mL) was added TFA (0.1 mL, 1.32 mmol). The reaction mixture was stirred at 25° C. for 1 h and then concentrated under vacuum. The residue was purified by preparative HPLC (column: Phenomenex luna C18 150*25 mm*10 μm; mobile phase: A: 0.225% formic acid in water; B %: 11%-41%, 5 min) to give the product 8-(3,6-diazabicyclo[3.2.1]octan-3-yl)-3-(5-(difluoromethyl)-1,3,4-thiadiazol-2-yl)-N-(1-methylcyclopropyl)imidazo[1,5-a]pyridine-6-sulfonamide formate (2.3 mg, 0.00408 mmol, 24.29% yield, FA salt) as a yellow solid.

RT 0.338 min (Method 3); m/z 496.1 (M+H)⁺ (ESI⁺);

¹H NMR (D₂O, 400 MHz): 9.42 (s, 1H), 8.39 (s, 1H), 7.84 (s, 1H), 7.28 (t, J=53.6 Hz, 1H), 6.59 (s, 1H), 4.29 (s, 1H), 3.92 (d, J=12.4 Hz, 1H), 3.71 (d, J=11.6 Hz, 2H), 3.46-3.31 (m, 1H), 3.24 (d, J=12.6 Hz, 1H), 3.11 (d, J=12.0 Hz, 1H), 2.90 (s, 1H), 2.12 (s, 2H), 1.13 (s, 3H), 0.72 (m, 2H), 0.46 (m, 2H).

It is noted that the compound may be a composition of isomers.

### Preparation of Example 184a

### tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-5,6-dihydropyridine-1(2H)-carboxylate

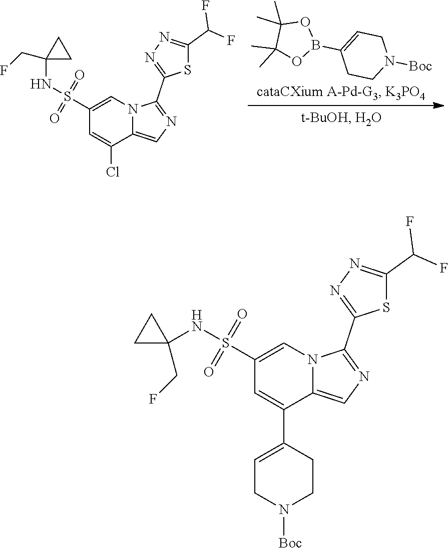

To a solution of 8-chloro-3-(5-(difluoromethyl)-1,3,4-thiadiazol-2-yl)-N-(1-(fluoromethyl)cyclopropyl)imidazo[1,5-a]pyridine-6-sulfonamide (100 mg, 0.228 mmol) in water (0.2 mL) and t-BuOH (2.0 mL) were added K₃PO₄ (48 mg, 0.228 mmol), cataCXium A-Pd-G3 (17 mg, 0.0228 mmol), tert-butyl 4-(4,4,5,5-tetramethyl-1,3,2-dioxaborolan-2-yl)-3,6-dihydro-2H-pyridine-1-carboxylate (212 mg, 0.685 mmol) at room temperature. The mixture was stirred at 60° C. for 16 h then diluted with water (20 mL) and extracted with EtOAc (50 mL, 3×). The combined organic layer was washed with brine (25 mL, 2×), dried over anhydrous Na₂SO₄, filtered and concentrated under vacuum. The residue was purified by preparative TLC (petroleum ether/EtOAc=2/1) to give the product tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-5,6-dihydropyridine-1(2H)-carboxylate (160 mg, 0.271 mmol, 118.63% yield) as a yellow solid.

RT 0.517 min (Method 3); m/z 585.1 (M+H)⁺ (ESI⁺)

### Preparation of Example 184b

### tert-butyl 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-5,6-dihydropyridine-1(2H)-carboxylate

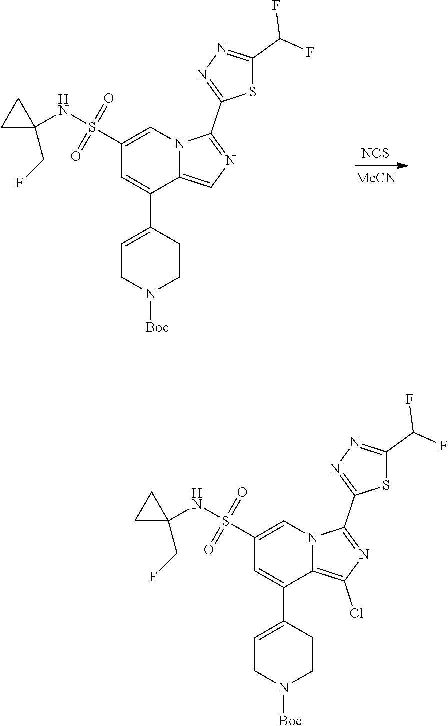 [SMILES: "MeCN" ; CC(C)(C)OC(=O)N1CC=C(c2cc(S(=O)(=O)NC3(CF)CC3)cn3c(-c4nnc(C(F)F)s4)ncc23)CC1 ; "NCS" ; CC(C)(C)OC(=O)N1CC=C(c2cc(S(=O)(=O)NC3(CF)CC3)cn3c(-c4nnc(C(F)F)s4)nc(Cl)c23)CC1]

To a solution of tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-5,6-dihydropyridine-1(2H)-carboxylate (160 mg, 0.271 mmol) in MeCN (2 mL) was added NCS (181 mg, 1.35 mmol) at 20° C. The mixture was stirred at 20° C. for 16 h, then diluted with NaHCO₃ (aq., sat., 20 mL) and extracted with EtOAc (30 mL, 3×). The combined organic layer was washed with brine (30 mL, 2×), dried over anhydrous Na₂SO₄, filtered and concentrated under vacuum. The residue was purified by preparative TLC (petroleum ether/EtOAc=2/1) to give the product tert-butyl 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-5,6-dihydropyridine-1(2H)-carboxylate (100 mg, 0.145 mmol, 53.66% yield) as a yellow solid.

RT 0.540 min (Method 3); m/z 619.1 (M+H)⁺ (ESI⁺); ¹H NMR (CDCl₃, 400 MHz): 10.13 (d, J=0.88 Hz, 1H), 7.22-6.95 (m, 2H), 5.88 (br s, 1H), 5.54 (s, 1H), 4.28 (d, J=48.4 Hz, 2H), 4.12 (m, 2H), 3.74 (t, J=5.4 Hz, 2H), 2.50 (br s, 2H), 1.53 (s, 9H), 1.12-1.20 (m, 2H), 0.86-0.93 (m, 2H).

### Preparation of Example 184

### 1-chloro-3-(5-(difluoromethyl)-1,3,4-thiadiazol-2-yl)-N-(1-(fluoromethyl)cyclopropyl)-8-(1,2,3,6-tetrahydropyridin-4-yl)imidazo[1,5-a]pyridine-6-sulfonamide formate

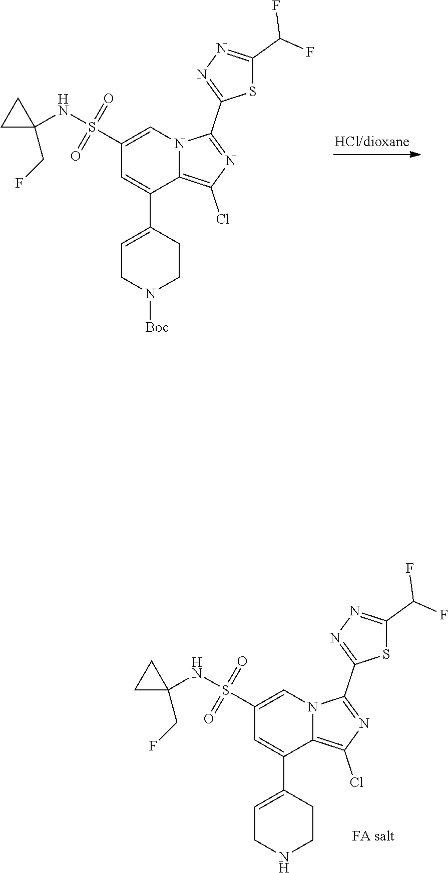 [SMILES: CC(C)(C)OC(=O)N1CC=C(c2cc(S(=O)(=O)NC3(CF)CC3)cn3c(-c4nnc(C(F)F)s4)nc(Cl)c23)CC1 ; O=S(=O)(NC1(CF)CC1)c1cc(C2=CCNCC2)c2c(Cl)nc(-c3nnc(C(F)F)s3)n2c1]

A solution of tert-butyl 4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-5,6-dihydropyridine-1(2H)-carboxylate (100 mg, 0.162 mmol) in HCl/dioxane (2.0 mL, 0.162 mmol) was stirred at 25° C. for 1 h. and, then s concentrated under vacuum. The residue was purified by preparative HPLC (column: Phenomenex luna C18 150*25 mm*10 μm; mobile phase: A: 0.225% formic acid in water, B: MeCN; B %: 15%-45%, 10 min) to give the product 1-chloro-3-(5-(difluoromethyl)-1,3,4-thiadiazol-2-yl)-N-(1-(fluoromethyl)cyclopropyl)-8-(1,2,3,6-tetrahydropyridin-4-yl)imidazo[1,5-a]pyridine-6-sulfonamide formate (27 mg, 0.0467 mmol, 28.92% yield, FA salt) as a yellow solid.

RT 0.540 min (Method 3); m/z 519.0 (M+H)⁺ (ESI⁺); ¹H NMR (CDCl₃, 400 MHz): 9.82 (s, 1H), 8.26 (s, 1H), 7.70 (t, J=53.2 Hz, 1H), 7.13 (s, 1H), 5.92 (br s, 1H), 4.24 (d, J=48.6 Hz, 2H), 3.54 (br s, 2H), 3.13 (br s, 2H), 2.40 (br s, 2H), 0.82-0.92 (m, 2H), 0.70-0.81 (m, 2H).

### Preparation of Example 185

### 1-chloro-8-(4-cyclopropylpiperazin-1-yl)-3-(5-(difluoromethyl)-1,3,4-thiadiazol-2-yl)-N-(1-(fluoromethyl)cyclopropyl)imidazo[1,5-a]pyridine-6-sulfonamide formate

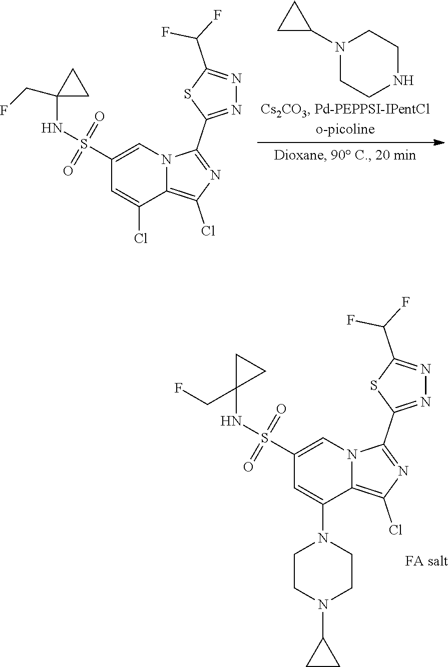 [SMILES: O=S(=O)(NC1(CF)CC1)c1cc(Cl)c2c(Cl)nc(-c3nnc(C(F)F)s3)n2c1 ; C1CN(C2CC2)CCN1 ; O=S(=O)(NC1(CF)CC1)c1cc(N2CCN(C3CC3)CC2)c2c(Cl)nc(-c3nnc(C(F)F)s3)n2c1]

To a solution of 1,8-dichloro-3-(5-(difluoromethyl)-1,3,4-thiadiazol-2-yl)-N-(1-(fluoromethyl)cyclopropyl)imidazo[1,5-a]pyridine-6-sulfonamide (30 mg, 63.52 μmol) in dioxane (1 mL) was added 1-cyclopropylpiperazine (16.03 mg, 127.04 μmol), Cs₂CO₃ (41.39 mg, 127.04 μmol) and Pd-PEPPSI-IPentCl o-picoline (6.18 mg, 6.35 μmol). The mixture was degassed with N₂ (3×), stirred at 90° C. for 20 min under a nitrogen atmosphere, cooled to 25° C., filtered and the filtrate was concentrated under vacuum. The residue was purified by preparative TLC (EtOAc:Petroleum ether=1:1) to give the impure product which was further purified by preparative HPLC (column: Unisil 3-100 C18 Ultra 150*25 mm*10 μm; mobile phase: A: 0.225% formic acid in water, B: MeCN; B %: 20%-50%, 10 min) and lyophilized directly to give the product 1-chloro-8-(4-cyclopropylpiperazin-1-yl)-3-(5-(difluoromethyl)-1,3,4-thiadiazol-2-yl)-N-(1-(fluoromethyl)cyclopropyl)imidazo[1,5-a]pyridine-6-sulfonamide formate (5.55 mg, 9.01 μmol, 14.18% yield, 98.67% purity, FA salt) as a yellow solid.

RT 0.354 min (Method 3), m/z 562.2 (M+H)⁺ (ESI⁺), ¹H NMR (DMSO-d₆, 400 MHz): 9.58 (d, J=0.8 Hz, 1H), 8.89 (br s, 1H), 8.25 (s, 1H) 7.68 (t, J=53.2 Hz, 1H), 6.79 (d, J=0.8 Hz, 1H), 4.22 (d, J=48.8 Hz, 2H), 3.13-2.94 (m, 4H), 2.90-2.71 (m, 4H), 1.82-1.69 (m, 1H), 0.92-0.82 (m, 2H), 0.81-0.72 (m, 2H), 0.53-0.42 (m, 2H), 0.42-0.31 (m, 2H).

### Preparation of Example 186a

### tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)piperazine-1-carboxylate

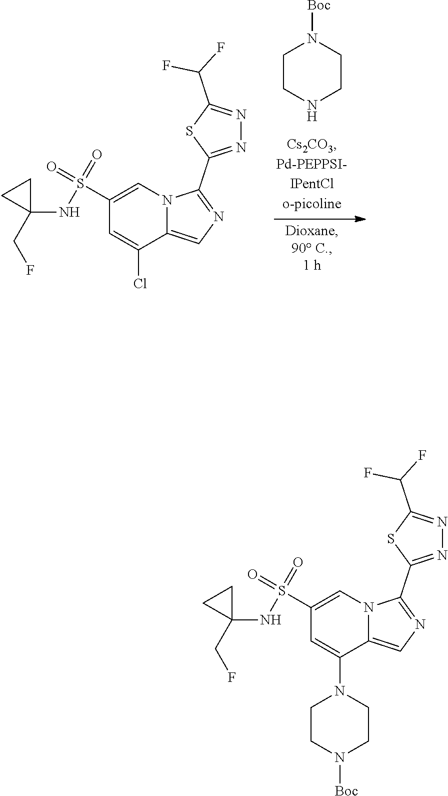 [SMILES: O=S(=O)(NC1(CF)CC1)c1cc(Cl)c2cnc(-c3nnc(C(F)F)s3)n2c1 ; CC(C)(C)OC(=O)N1CCNCC1 ; CC(C)(C)OC(=O)N1CCN(c2cc(S(=O)(=O)NC3(CF)CC3)cn3c(-c4nnc(C(F)F)s4)ncc23)CC1]

To a solution of 8-chloro-3-(5-(difluoromethyl)-1,3,4-thiadiazol-2-yl)-N-(1-(fluoromethyl)cyclopropyl)imidazo[1,5-a]pyridine-6-sulfonamide (50 mg, 114.20 μmol) in dioxane (2 mL) were added tert-butyl piperazine-1-carboxylate (42.54 mg, 228.39 μmol), Cs₂CO₃ (74.41 mg, 228.39 μmol) and Pd-PEPPSI-IPentCl o-picoline (11.11 mg, 11.42 μmol) at room temperature. The mixture was degassed with N₂ (3×), stirred at 90° C. for 1 h under a nitrogen atmosphere, cooled to 25° C. and filtered. The filtrate was concentrated under vacuum to give a residue, which was purified by preparative TLC (EtOAc:Petroleum ether=2:1) to give the product tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)piperazine-1-carboxylate ((50 mg, 75.73 μmol, 33.16% yield, 89% purity) as a yellow solid.

RT 0.531 min (Method 3), m/z 588.2 (M+H)⁺ (ESI⁺), ¹H NMR (CDCl₃, 400 MHz): 9.89 (s, 1H), 7.72 (s, 1H), 7.08 (t, J=53.6 Hz, 1H), 6.64 (s, 1H), 5.49 (s, 1H), 4.27 (d, J=48.4 Hz, 2H), 3.76-3.64 (m, 4H), 3.39-3.23 (m, 4H), 1.52 (s, 9H), 1.20-1.13 (m, 2H), 0.93-0.84 (m, 2H).

### Preparation of Example 187

### 3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide formate

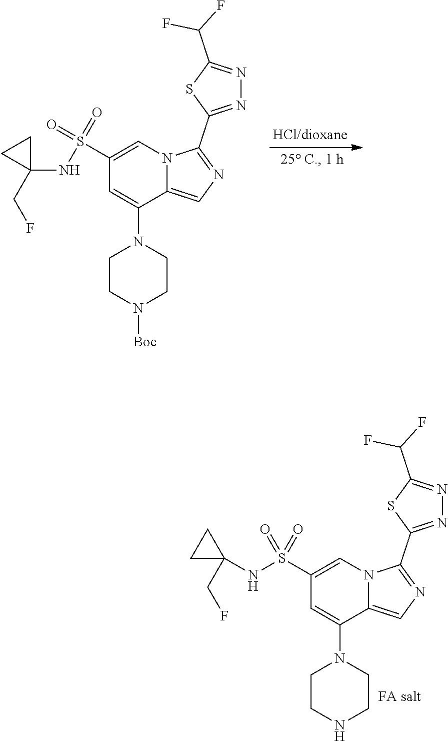 [SMILES: CC(C)(C)OC(=O)N1CCN(c2cc(S(=O)(=O)NC3(CF)CC3)cn3c(-c4nnc(C(F)F)s4)ncc23)CC1 ; O=S(=O)(NC1(CF)CC1)c1cc(N2CCNCC2)c2cnc(-c3nnc(C(F)F)s3)n2c1]

A solution of tert-butyl 4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)piperazine-1-carboxylate (45 mg, 76.58 μmol) in HCl/dioxane (4 M, 1 mL) was stirred at 25° C. for 1 h and concentrated under vacuum. The residue was purified by preparative HPLC (column: Unisil 3-100 C18 Ultra 150*25 mm*10 μm; mobile phase: A: 0.225% formic acid in water, B: MeCN; B %: 12%-42%, 10 min) and lyophilized directly to give the product 3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide (22.23 mg, 40.19 μmol, 52.48% yield, 96.45% purity, FA salt) as a yellow solid.

RT 0.324 min (Method 3); m/z 488.1 (M+H)⁺ (ESI⁺); ¹H NMR (DMSO-d₆, 400 MHz): 9.56 (s, 1H), 8.92-8.71 (m, 1H), 8.23 (s, 1H), 7.99 (s, 1H), 7.67 (t, J=53.2 Hz, 1H), 6.67 (s, 1H), 4.22 (d, J=48.4 Hz, 2H), 3.25-3.20 (m, 4H), 3.05-2.92 (m, 4H), 0.88-0.81 (m, 2H), 0.80-0.74 (m, 2H).

### Preparation of Example 188

### 3-(5-(difluoromethyl)-1,3,4-thiadiazol-2-yl)-N-(1-(fluoromethyl)cyclopropyl)-8-(4-isobutyrylpiperazin-1-yl)imidazo[1,5-a]pyridine-6-sulfonamide

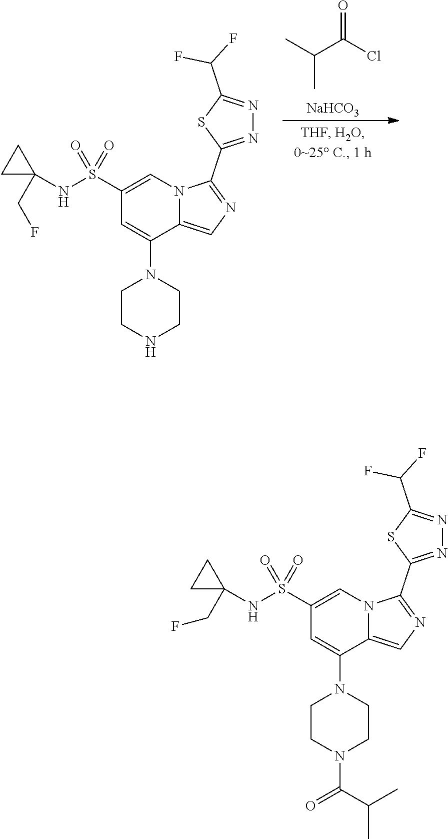

To a solution of 3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide (15 mg, 30.77 μmol, FA salt) in THE (0.5 mL) and H₂O (0.5 mL) was added NaHCO₃ (25.85 mg, 307.68 μmol), followed by isobutyryl chloride (4.92 mg, 46.15 μmol, 4.82 μL) at 0° C. The mixture was stirred at 25° C. for 1 h, then poured into water (10 mL) and extracted with EtOAc (10 mL; 2×). The organic layer was collected, dried over anhydrous Na₂SO₄, filtered and concentrated under vacuum. The residue was purified by preparative TLC (petroleum ether:EtOAc=1:1) to give the impure product, which further purified by trituration with MeOH (2 mL) at 25° C. for 20 min. After filtration, the cake was collected and dried under vacuum to give the product 3-(5-(difluoromethyl)-1,3,4-thiadiazol-2-yl)-N-(1-(fluoromethyl)cyclopropyl)-8-(4-isobutyrylpiperazin-1-yl)imidazo[1,5-a]pyridine-6-sulfonamide (6.43 mg, 11.35 μmol, 36.90% yield, 98.46% purity) as a yellow solid.

RT 0.485 min (Method 3); m/z 558.3 (M+H)⁺ (ESI⁺); ¹H NMR (CDCl₃, 400 MHz): 9.91-9.90 (m, 1H), 7.74 (s, 1H), 7.08 (t, J=53.2 Hz, 1H), 6.65 (d, J=1.2 Hz, 1H), 5.50 (s, 1H), 4.27 (d, J=48.8 Hz, 2H), 3.98-3.75 (m, 4H), 3.45-3.25 (m, 4H), 2.90-2.78 (m, 1H), 1.20 (d, J=6.8 Hz, 6H), 1.18-1.14 (m, 2H), 0.90-0.86 (m, 2H).

### Preparation of Example 189a

### tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)piperazine-1-carboxylate

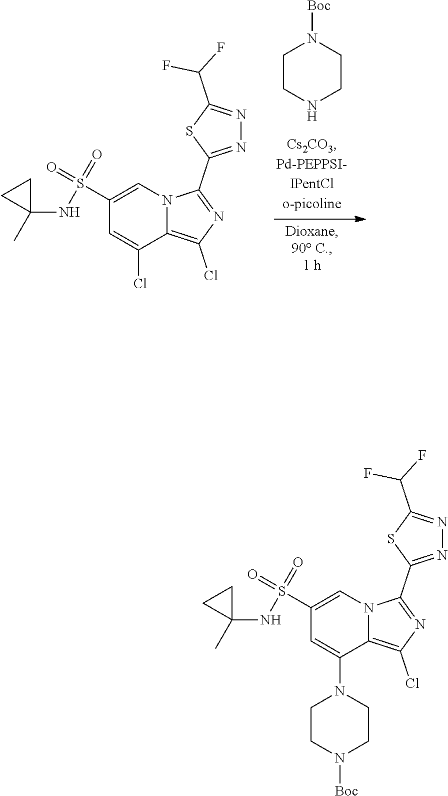 [SMILES: CC1(NS(=O)(=O)c2cc(Cl)c3c(Cl)nc(-c4nnc(C(F)F)s4)n3c2)CC1 ; CC(C)(C)OC(=O)N1CCNCC1 ; CC1(NS(=O)(=O)c2cc(N3CCN(C(=O)OC(C)(C)C)CC3)c3c(Cl)nc(-c4nnc(C(F)F)s4)n3c2)CC1]

To a solution of 1,8-dichloro-3-[5-(difluoromethyl)-1,3,4-thiadiazol-2-yl]-N-(1-methylcyclopropyl)imidazo[1,5-a]pyridine-6-sulfonamide (50 mg, 0.110 mmol) in dioxane (2 mL) was added tert-butyl piperazine-1-carboxylate (41 mg, 0.220 mmol), Pd-PEPPSI-IPentCl o-picoline (11 mg, 0.0110 mmol) and Cs₂CO₃ (90 mg, 0.275 mmol). The mixture was degassed with N₂ (3×), stirred at 90° C. for 1 h under nitrogen atmosphere, cooled to 25° C. and filtered. The filtrate was concentrated under vacuum to give a residue which was purified by preparative TLC (EtOAc:Petroleum ether=2:1) to give the product tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)piperazine-1-carboxylate (18 mg, 0.0275 mmol, 24.99% yield) as a yellow solid.

RT 0.535 min (Method 3), m/z 604.0 (M+H)⁺ (ESI⁺), ¹H NMR (CDCl₃, 400 MHz): 9.90 (s, 1H), 7.08 (t, J=54.0 Hz, 1H), 6.73 (s, 1H), 5.07 (s, 1H), 3.84-3.58 (m, 4H), 3.24-3.02 (m, 4H), 1.51 (s, 9H), 1.39 (s, 3H), 0.94-0.90 (m, 2H), 0.63-0.61 (m, 2H).

### Preparation of Example 189

### 1-chloro-3-(5-(difluoromethyl)-1,3,4-thiadiazol-2-yl)-N-(1-methylcyclopropyl)-8-(piperazin-1-yl)imidazo[1,5-a]pyridine-6-sulfonamide formate

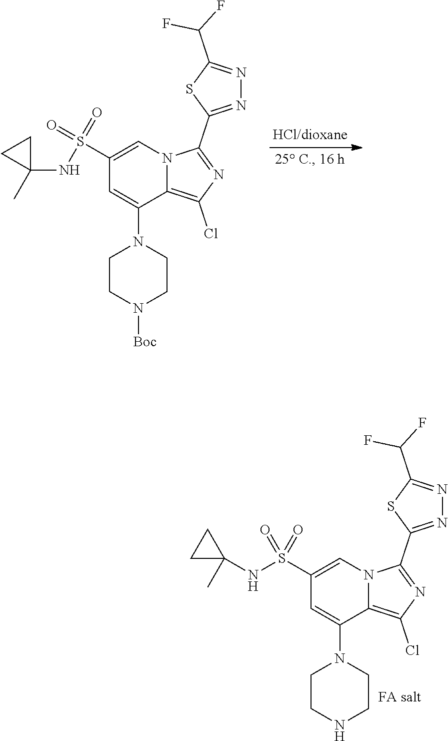 [SMILES: CC1(NS(=O)(=O)c2cc(N3CCN(C(=O)OC(C)(C)C)CC3)c3c(Cl)nc(-c4nnc(C(F)F)s4)n3c2)CC1 ; CC1(NS(=O)(=O)c2cc(N3CCNCC3)c3c(Cl)nc(-c4nnc(C(F)F)s4)n3c2)CC1]

A solution of tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)piperazine-1-carboxylate (12 mg, 0.0199 mmol) in HCl/dioxane (4 M, 1 mL) was stirred at 25° C. for 16 h. The reaction mixture was concentrated under vacuum to give a residue which was purified by preparative HPLC (column: Unisil 3-100 C18 Ultra 150*50 mm*10 μm; mobile phase: A: 0.225% formic acid in water, B: MeCN; B %: 15%-45%, 10 min) and lyophilized directly to give the product 1-chloro-3-(5-(difluoromethyl)-1,3,4-thiadiazol-2-yl)-N-(1-methylcyclopropyl)-8-(piperazin-1-yl)imidazo[1,5-a]pyridine-6-sulfonamide formate (7.26 mg, 0.0132 mmol, 66.45% yield, FA salt) as a yellow solid.

RT 0.340 min (Method 3); m/z 504.1 (M+H)⁺ (ESI⁺); ¹H NMR (DMSO-d₆, 400 MHz): 9.61 (s, 1H), 8.61-8.44 (m, 1H), 8.20 (s, 1H), 7.69 (t, J=53.2 Hz, 1H), 6.81 (s, 1H), 3.08-3.02 (m, 4H), 3.02-2.97 (m, 4H), 1.18 (s, 3H), 0.82-0.66 (m, 2H), 0.56-0.36 (m, 2H).

### Preparation of Example 190a

### tert-butyl 2-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2,6-diazaspiro[3.4]octane-6-carboxylate

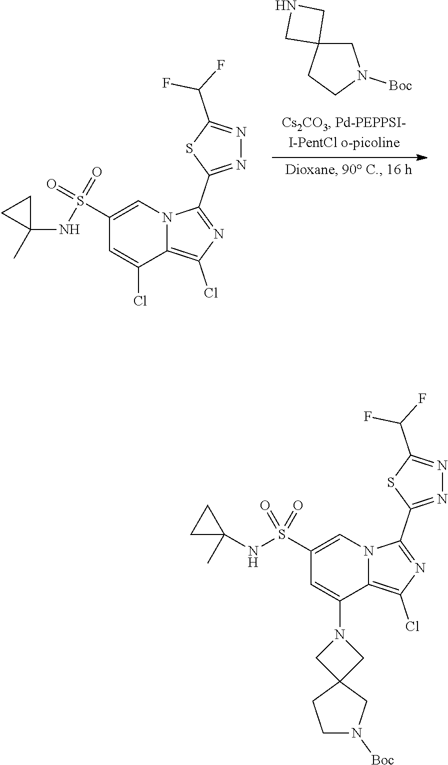 [SMILES: CC1(NS(=O)(=O)c2cc(Cl)c3c(Cl)nc(-c4nnc(C(F)F)s4)n3c2)CC1 ; CC(C)(C)OC(=O)N1CCC2(CNC2)C1 ; CC1(NS(=O)(=O)c2cc(N3CC4(CCN(C(=O)OC(C)(C)C)C4)C3)c3c(Cl)nc(-c4nnc(C(F)F)s4)n3c2)CC1]

To a solution of 1,8-dichloro-3-(5-(difluoromethyl)-1,3,4-thiadiazol-2-yl)-N-(1-methylcyclopropyl)imidazo[1,5-a]pyridine-6-sulfonamide (50 mg, 0.110 mmol) in dioxane (0.5 mL) were added tert-butyl 2,6-diazaspiro[3.4]octane-6-carboxylate (47 mg, 0.220 mmol), Cs₂CO₃ (108 mg, 0.330 mmol) and Pd-PEPPSI-IPentCl o-picoline (11 mg, 0.0110 mmol) at room temperature. The mixture was degassed with N₂ (3×), stirred at 98° C. for 1 h under nitrogen atmosphere, cooled to 25° C. and filtered. The filtrate was concentrated under vacuum to give a residue, which was purified by preparative TLC (EtOAc:Petroleum ether=1:1) to give the product tert-butyl 2-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2,6-diazaspiro[3.4]octane-6-carboxylate (32 mg, 0.0486 mmol, 44.14% yield) as a yellow solid.

RT 0.531 min (Method 3), m/z 630.3 (M+H)⁺ (ESI⁺), ¹H NMR (CDCl₃, 400 MHz): 9.82 (s, 1H), 7.08 (t, J=53.6 Hz, 1H), 6.60 (s, 1H), 5.08 (s, 1H), 4.04-3.98 (m, 2H), 3.98-3.91 (m, 2H), 3.61 (s, 2H), 3.51 (t, J=6.8 Hz, 2H), 2.29 (t, J=6.8 Hz, 2H), 1.46 (s, 9H), 1.39 (s, 3H), 0.97-0.90 (m, 2H), 0.65-0.55 (m, 2H).

### Preparation of Example 190

### 1-chloro-3-(5-(difluoromethyl)-1,3,4-thiadiazol-2-yl)-N-(1-methylcyclopropyl)-8-(2,6-diazaspiro[3.4]octan-2-yl)imidazo[1,5-a]pyridine-6-sulfonamide formate

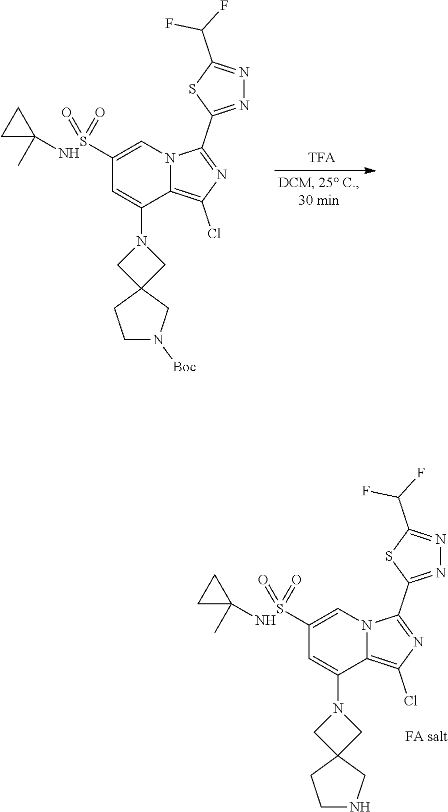

To a solution of tert-butyl 2-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2,6-diazaspiro[3.4]octane-6-carboxylate (32 mg, 0.0486 mmol) in DCM (0.5 mL) was added TFA (0.1 mL, 0.250 mmol) at 0° C. The mixture was stirred at 25° C. for 30 min and then, concentrated under vacuum. The residue, was purified by preparative HPLC (column: Unisil 3-100 C18 Ultra 150*50 mm*10 μm; mobile phase: A: 0.225% formic acid in water, B: MeCN; B %: 16%-46%, 10 min) and lyophilized directly to give the product 1-chloro-3-(5-(difluoromethyl)-1,3,4-thiadiazol-2-yl)-N-(1-methylcyclopropyl)-8-(2,6-diazaspiro[3.4]octan-2-yl)imidazo[1,5-a]pyridine-6-sulfonamide formate (9.97 mg, 0.0167 mmol, 34.47% yield, FA salt) as a yellow solid.

RT 0.340 min (Method 3); m/z 530.0 (M+H)⁺ (ESI⁺); ¹H NMR (DMSO-d₆, 400 MHz): 9.55 (s, 1H), 9.01-8.11 (m, 2H), 7.69 (t, J=53.2 Hz, 1H), 6.67 (s, 1H), 3.93-3.79 (m, 4H), 3.64-3.58 (m, 4H), 2.26 (t, J=6.8 Hz, 2H), 1.18 (s, 3H), 0.77-0.67 (m, 2H), 0.49-0.39 (m, 2H).

### Preparation of Example 191a

### (S)-tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate

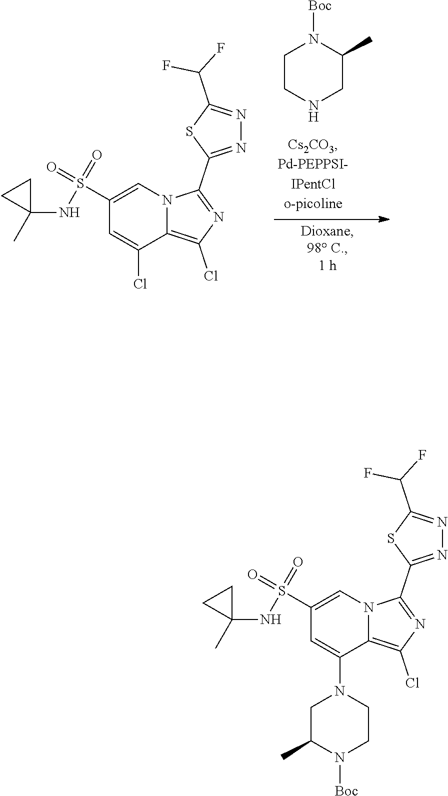 [SMILES: CC1(NS(=O)(=O)c2cc(Cl)c3c(Cl)nc(-c4nnc(C(F)F)s4)n3c2)CC1 ; C[C@H]1CNCCN1C(=O)OC(C)(C)C ; C[C@H]1CN(c2cc(S(=O)(=O)NC3(C)CC3)cn3c(-c4nnc(C(F)F)s4)nc(Cl)c23)CCN1C(=O)OC(C)(C)C]

To a solution of 1,8-dichloro-3-[5-(difluoromethyl)-1,3,4-thiadiazol-2-yl]-N-(1-methylcyclopropyl)imidazo[1,5-a]pyridine-6-sulfonamide (50 mg, 0.110 mmol) in dioxane (2 mL) were added (S)-tert-butyl 2-methylpiperazine-1-carboxylate (88 mg, 0.440 mmol), Cs₂CO₃ (108 mg, 0.330 mmol) and Pd-PEPPSI-IPentCl o-picoline (11 mg, 0.0110 mmol). The mixture was degassed with N₂ (3×), stirred at 98° C. for 1 h under a nitrogen atmosphere, cooled to 25° C. and filtered. The filtrate was concentrated under vacuum to give a residue, which was purified by preparative TLC (Ethyl acetate:Petroleum ether=1:1) to give the product (S)-tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (10 mg, 0.0121 mmol, 10.96% yield, 74.56% purity) as a yellow solid.

RT 0.552 min (method 4), m/z 618.2 (M+H)⁺ (ESI⁺).

### Preparation of Example 191

### (S)-1-chloro-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate

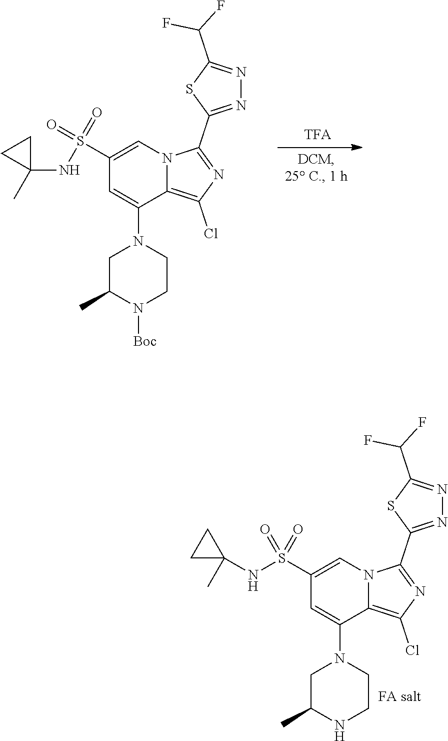 [SMILES: C[C@H]1CN(c2cc(S(=O)(=O)NC3(C)CC3)cn3c(-c4nnc(C(F)F)s4)nc(Cl)c23)CCN1C(=O)OC(C)(C)C ; C[C@H]1CN(c2cc(S(=O)(=O)NC3(C)CC3)cn3c(-c4nnc(C(F)F)s4)nc(Cl)c23)CCN1]

To a solution of (S)-tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (8.0 mg, 0.00965 mmol) in DCM (1.5 mL) was added TFA (0.3 mL). The mixture was stirred at 25° C. for 1 h and then, concentrated under vacuum. The residue was purified by preparative HPLC (column: Phenomenex luna C18 150*25 mm*10 um; mobile phase: A: 0.225% formic acid in water, B: MeCN; B %: 16%-46%, 10 min) and lyophilized directly to give the product (S)-1-chloro-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate (1.38 mg, 0.00243 mmol, 25.17% yield, 99.28% purity, FA salt) as a yellow solid.

RT 0.377 min (method 4); m/z 518.2 (M+H)⁺ (ESI⁺); ¹H NMR (CDCl₃, 400 MHz): 9.91 (s, 1H), 8.26 (s, 1H), 7.08 (t, J=53.6 Hz, 1H), 6.81 (s, 1H), 5.44 (s, 1H), 3.56-3.26 (m, 5H), 3.18-3.03 (m, 1H), 2.88-2.69 (m, 1H), 1.38 (s, 3H), 1.35 (d, J=6.0 Hz, 3H), 0.96-0.90 (m, 2H), 0.65-0.58 (m, 2H).

### Preparation of Example 192a

### (R)-tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate

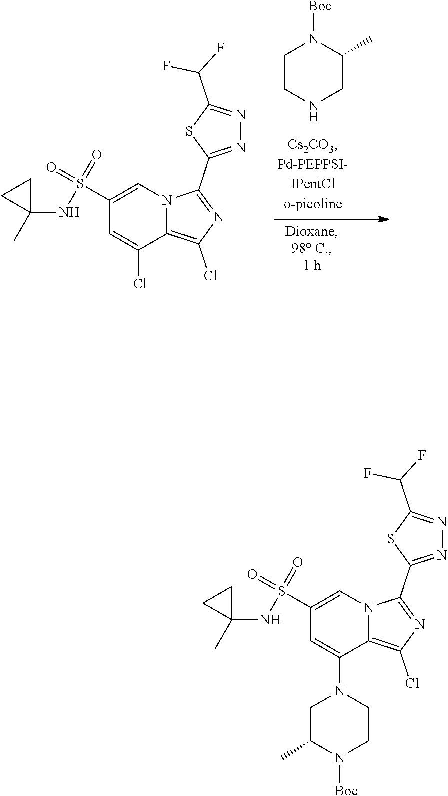 [SMILES: CC1(NS(=O)(=O)c2cc(Cl)c3c(Cl)nc(-c4nnc(C(F)F)s4)n3c2)CC1 ; C[C@@H]1CNCCN1C(=O)OC(C)(C)C ; C[C@@H]1CN(c2cc(S(=O)(=O)NC3(C)CC3)cn3c(-c4nnc(C(F)F)s4)nc(Cl)c23)CCN1C(=O)OC(C)(C)C]

To a solution of 1,8-dichloro-3-[5-(difluoromethyl)-1,3,4-thiadiazol-2-yl]-N-(1-methylcyclopropyl)imidazo[1,5-a]pyridine-6-sulfonamide (50 mg, 0.110 mmol) in dioxane (2 mL) was added (R)-tert-butyl 2-methylpiperazine-1-carboxylate (88 mg, 0.440 mmol), Cs₂CO₃ (108 mg, 0.330 mmol) and Pd-PEPPSI-IPentCl o-picoline (11 mg, 0.0110 mmol). The mixture was degassed with N₂ (3×), stirred at 98° C. for 1 h under N₂ atmosphere, cooled to 25° C. and filtered. The filtrate was concentrated under vacuum to give a residue which was purified by preparative TLC (Ethyl acetate:Petroleum ether=1:1) to give the product (R)-tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (10 mg, 0.0148 mmol, 13.45% yield, 91.50% purity) as a yellow solid.

RT 0.546 min (method 4), m/z 618.2 (M+H)⁺ (ESI⁺).

### Preparation of Example 192

### (R)-1-chloro-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide 2,2,2-trifluoroacetate

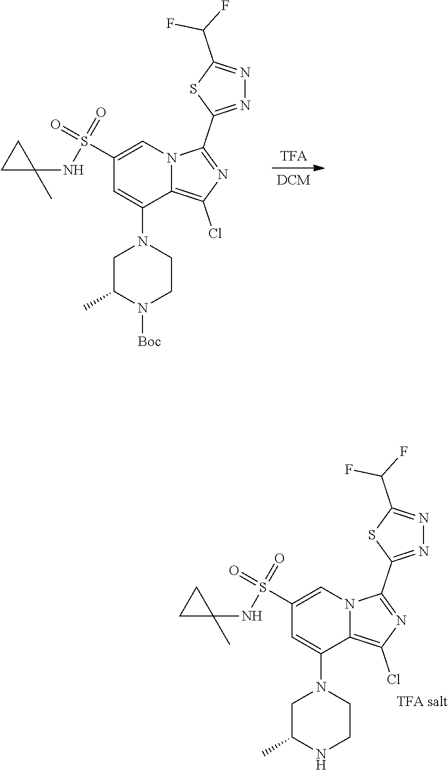 [SMILES: C[C@@H]1CN(c2cc(S(=O)(=O)NC3(C)CC3)cn3c(-c4nnc(C(F)F)s4)nc(Cl)c23)CCN1C(=O)OC(C)(C)C ; C[C@@H]1CN(c2cc(S(=O)(=O)NC3(C)CC3)cn3c(-c4nnc(C(F)F)s4)nc(Cl)c23)CCN1]

To a solution of (R)-tert-butyl 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (8.0 mg, 0.0118 mmol) in DCM (1.5 mL) was added TFA (0.5 mL). The mixture was stirred at 25° C. for 1 h and concentrated under vacuum. The resulting residue was purified by preparative HPLC (column: Phenomenex luna C18 150*25 mm*10 um; mobile phase: A: 0.1% TFA in water, B: MeCN; B %: 25%-55%, 10 min) and lyophilized directly to give the product (R)-1-chloro-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide 2,2,2-trifluroacetate (7.33 mg, 0.00978 mmol, 82.56% yield, 99.51% purity, 2 TFA salt) as a yellow solid.

RT 0.383 min (method 4); m/z 518.1 (M+H)⁺ (ESI⁺); ¹H NMR (CDCl₃, 400 MHz): 10.01 (br, 1H), 9.94 (s, 1H), 9.37 (br, 1H), 7.09 (t, J=53.6 Hz, 1H), 6.89 (s, 1H), 5.93 (s, 1H), 3.85-3.70 (m, 1H), 3.69-3.51 (m, 3H), 3.50-3.24 (m, 2H), 3.19-3.03 (m, 1H), 1.51 (d, J=6.4 Hz, 3H), 1.35 (s, 3H), 1.01-0.82 (m, 2H), 0.66-0.49 (m, 2H).

### Preparation of Intermediate 193.1

### 1,8-dichloro-3-(5-(difluoromethyl)thiazol-2-yl)imidazo[1,5-a]pyridine-6-sulfonyl chloride

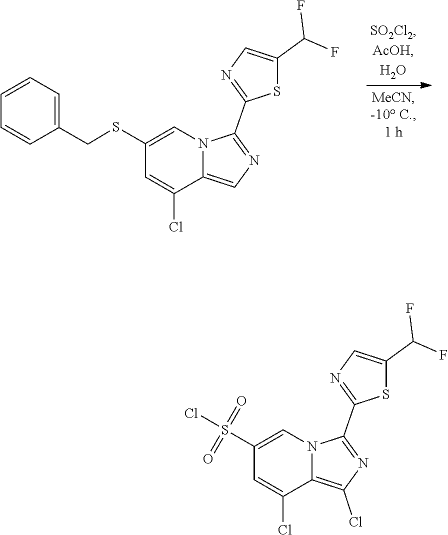 [SMILES: FC(F)c1cnc(-c2ncc3c(Cl)cc(SCc4ccccc4)cn23)s1 ; O=S(=O)(Cl)c1cc(Cl)c2c(Cl)nc(-c3ncc(C(F)F)s3)n2c1]

To a mixture of 2-(6-(benzylthio)-8-chloroimidazo[1,5-a]pyridin-3-yl)-5-(difluoromethyl)thiazole (65 mg, 159.36 μmol) in MeCN (2 mL) was added AcOH (19.14 mg, 318.72 μmol, 18.23 μL) and H₂O (5.74 mg, 318.72 μmol, 5.74 μL) at room temperature. The mixture was cooled to −10° C. and sulfuryl dichloride (75.28 mg, 557.75 μmol, 55.76 μL) was added. The reaction mixture was stirred at −10° C. for 1 h and then quenched with ice water (8 mL). The yellow precipitate was collected by filtration and dried under vacuum to give the crude product 1,8-dichloro-3-(5-(difluoromethyl)thiazol-2-yl)imidazo[1,5-a]pyridine-6-sulfonyl chloride (65 mg, crude) which used directly in next step without any further purification.

### Preparation of Example 193

### 1,8-dichloro-3-(5-(difluoromethyl)thiazol-2-yl)-N-(1-(fluoromethyl)cyclopropyl)imidazo[1,5-a]pyridine-6-sulfonamide

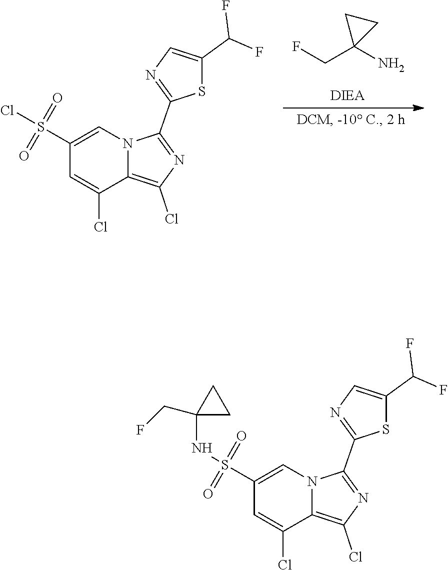

To a mixture of 1-(fluoromethyl)cyclopropanamine (22.50 mg, 138.83 μmol, HCl salt) in DCM (1 mL) was added DIEA (46.31 mg, 358.29 μmol, 62.41 μL) and 1,8-dichloro-3-[5-(difluoromethyl)thiazol-2-yl]imidazo[1,5-a]pyridine-6-sulfonyl chloride (50 mg, 119.43 μmol) at −10° C. The reaction mixture was stirred at −10° C. for 2 h, then quenched with brine (5 mL) and NaHCO₃ (aq., sat., 5 mL) and extracted with EtOAc (12 mL; 2×). The organic layers were separated, combined, dried over anhydrous Na₂SO₄, filtered and concentrated under vacuum. The residue was purified by preparative TLC (Petroleum ether/EtOAc=3/1) to give the product 1,8-dichloro-3-[5-(difluoromethyl)thiazol-2-yl]-N-[1-(fluoromethyl)cyclopropyl]imidazo[1,5-a]pyridine-6-sulfonamide (35 mg, 69.65 μmol, 58.32% yield, 93.79% purity) as a yellow solid.

RT 0.531 min (Method 3); m/z 471.0 (M+H)⁺ (ESI⁺)

### Preparation of Example 194

### 4-(1-chloro-3-(5-(difluoromethyl)thiazol-2-yl)-6-(N-(1-(fluoromethyl)cyclopropyl)sulfamoyl)imidazo[1,5-a]pyridin-8-yl)-N,N-dimethylpiperazine-1-carboxamide

### & Example 195

### 4-(3-(5-(difluoromethyl)thiazol-2-yl)-6-(N-(1-(fluoromethyl)cyclopropyl)sulfamoyl)imidazo[1,5-a]pyridin-8-yl)-N,N-dimethylpiperazine-1-carboxamide

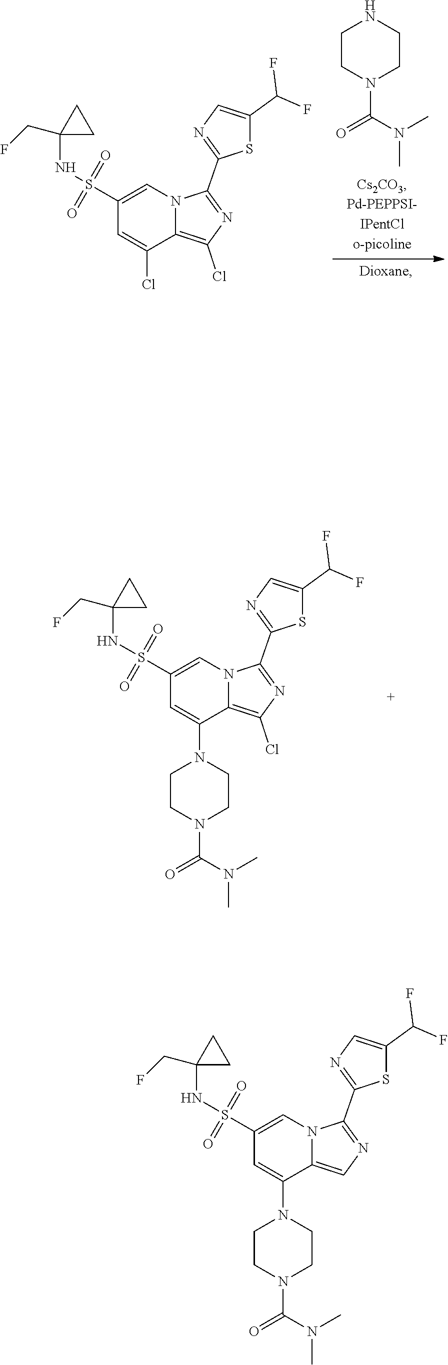

A mixture of 1,8-dichloro-3-[5-(difluoromethyl)thiazol-2-yl]-N-[1-(fluoromethyl)cyclopropyl]imidazo[1,5-a]pyridine-6-sulfonamide (20 mg, 42.44 μmol), N,N-dimethylpiperazine-1-carboxamide (13.34 mg, 84.87 μmol), Pd-PEPPSI-IPentCl o-picoline (3.65 mg, 4.24 μmol) and Cs₂CO₃ (41.48 mg, 127.31 μmol) in dioxane (0.5 mL) was degassed with N₂ (3×). Then, the mixture was stirred at 90° C. for 1 h, then cooled to room temperature, filtered and the filtrate was concentrated under vacuum. The residue was purified by preparative TLC (Petroleum ether:EtOAc=0:1) to give the product 4-(1-chloro-3-(5-(difluoromethyl)thiazol-2-yl)-6-(N-(1-(fluoromethyl)cyclopropyl)sulfamoyl)imidazo[1,5-a]pyridin-8-yl)-N,N-dimethylpiperazine-1-carboxamide (2.64 mg, 4.22 μmol, 9.94% yield, 94.74% purity) as a yellow solid,

and an impure fraction (10 mg, 80% purity) which was further purified by preparative HPLC (column: Unisil 3-100 C18 Ultra 150*50 mm*3 μm; mobile phase: A: 0.225% formic acid in water, B: MeCN; 38%-68%, 10 min) and lyophilized directly to give the product 4-(3-(5-(difluoromethyl)thiazol-2-yl)-6-(N-(1-(fluoromethyl)cyclopropyl)sulfamoyl)imidazo[1,5-a]pyridin-8-yl)-N,N-dimethylpiperazine-1-carboxamide (0.87 mg, 0.00149 mmol, 8.33% yield) as a yellow solid

### 4-(1-chloro-3-(5-(difluoromethyl)thiazol-2-yl)-6-(N-(1-(fluoromethyl)cyclopropyl)sulfamoyl)imidazo[1,5-a]pyridin-8-yl)-N,N-dimethylpiperazine-1-carboxamide

RT 0.492 min (Method 3); m/z 592.2 (M+H)⁺ (ESI⁺); ¹H NMR (CDCl₃, 400 MHz): 9.95 (s, 1H), 8.07 (s, 1H), 6.97 (t, J=55.2 Hz, 1H), 6.60 (s, 1H), 5.45 (s, 1H), 4.26 (d, J=52.4 Hz, 2H), 3.62-3.43 (m, 4H), 3.27-3.06 (m, 4H), 2.90 (s, 6H), 1.13-1.12 (m, 2H), 0.87-0.84 (m, 2H).

### 4-(3-(5-(difluoromethyl)thiazol-2-yl)-6-(N-(1-(fluoromethyl)cyclopropyl)sulfamoyl)imidazo[1,5-a]pyridin-8-yl)-N,N-dimethylpiperazine-1-carboxamide

RT 0.459 min (Method 3), m/z 558.3 (M+H)⁺ (ESI⁺); ¹H NMR (CDCl₃, 400 MHz), 9.94 (s, 1H), 8.07 (s, 1H), 7.63 (s, 1H), 6.97 (t, J=53.2 Hz, 1H), 6.53 (s, 1H), 5.45 (s, 1H), 4.26 (d, J=48.4 Hz, 2H), 3.61-3.43 (m, 4H), 3.42-3.22 (m, 4H), 2.91 (s, 6H), 1.14-1.11 (m, 2H), 0.86-0.81 (m, 2H).

### Preparation of Example 196

### 1-chloro-3-(5-(difluoromethyl)thiazol-2-yl)-N-(1-(fluoromethyl)cyclopropyl)-8-(2-oxa-7-azaspiro[3.5]nonan-7-yl)imidazo[1,5-a]pyridine-6-sulfonamide

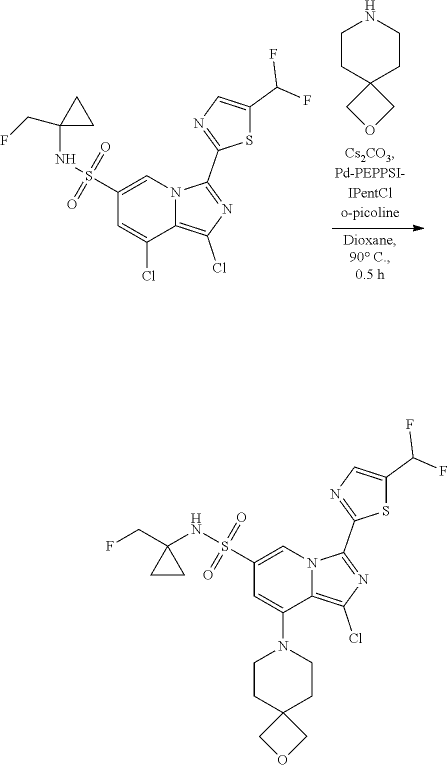

To a mixture of 1,8-dichloro-3-[5-(difluoromethyl)thiazol-2-yl]-N-[1-(fluoromethyl)cyclopropyl]imidazo[1,5-a]pyridine-6-sulfonamide (15 mg, 31.83 μmol) in dioxane (0.5 mL) was added 2-oxa-7-azaspiro[3.5]nonane (8.10 mg, 63.65 μmol), Cs₂CO₃ (31.11 mg, 95.48 μmol) and Pd-PEPPSI-IPentCl o-picoline (2.74 mg, 3.18 μmol) and the reaction mixture was degassed with N₂ (3×). The mixture was stirred at 90° C. for 30 min then, cooled to room temperature, filtered and concentrated under vacuum. The residue was purified by preparative TLC (EtOAc:Petroleum ether=3:1) followed by preparative HPLC (column: Phenomenex luna C18 150*25 mm*10 μm; mobile phase: A: 0.225% formic acid in water; B: MeCN; B %: 50%-80%, 10 min) and lyophilization to give the product 1-chloro-3-[5-(difluoromethyl)thiazol-2-yl]-N-[1-(fluoromethyl)cyclopropyl]-8-(2-oxa-7-azaspiro[3.5]nonan-7-yl)imidazo[1,5-a]pyridine-6-sulfonamide (1.71 mg, 2.83 μmol, 8.89% yield, 93.04% purity) as a yellow solid.

RT 0.514 min (Method 3), m/z 562.3 (M+H)⁺ (ESI⁺); ¹H NMR (CDCl₃, 400 MHz), 9.92 (s, 1H), 8.06 (s, 1H), 6.97 (t, J=55.2 Hz, 1H), 6.56 (s, 1H), 5.44 (s, 1H), 4.53 (s, 4H), 4.25 (d, J=48.4, 2H), 3.16-2.93 (m, 4H), 2.31-2.06 (m, 4H), 1.13-1.11 (m, 2H), 0.87-0.82 (m, 2H).

### Preparation of Example 197a

### (R)-tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate

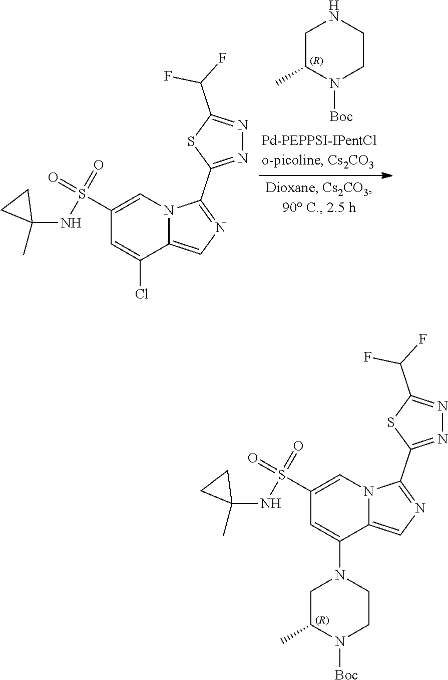

To a mixture of 8-chloro-3-(5-(difluoromethyl)-1,3,4-thiadiazol-2-yl)-N-(1-methylcyclopropyl)imidazo[1,5-a]pyridine-6-sulfonamide (80 mg, 0.191 mmol) in dioxane (1.5 mL) was added tert-butyl tert-butyl (R)-2-methylpiperazine-1-carboxylate (153 mg, 0.762 mmol), Cs₂CO₃ (217 mg, 0.667 mmol) and Pd-PEPPSI-IPentCl o-picoline (19 mg, 0.0191 mmol). The mixture was degassed and purged with N₂ (3×) and then stirred at 90° C. for 2 h under N₂ atmosphere. The reaction mixture was filtered and the filtrate was concentrated under vacuum. The residue was purified by preparative TLC (Petroleum ether:EtOAc=2:1) to give the product tert-butyl (R)-4-(3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (28 mg, 0.0437 mmol, 31.85% yield) as a yellow solid.

RT 0.539 min (Method 3); m/z 584.4 (M+H)⁺ (ESI⁺); ¹H NMR (CDCl₃, 400 MHz) 9.87 (s, 1H), 7.73 (s, 1H), 7.08 (t, J=53.6 Hz, 1H), 6.64 (s, 1H), 5.10 (s, 1H), 4.46 (br s, 1H), 4.08 (d, J=12.8 Hz, 1H), 3.62-3.53 (m, 2H), 3.40-3.33 (m, 1H), 3.07-2.95 (m, 2H), 1.51 (s, 9H), 1.47 (d, J=6.8 Hz, 3H), 1.39 (s, 3H), 0.97-0.90 (m, 2H), 0.65-0.57 (m, 2H).

### Preparation of Example 197

### (R)-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate

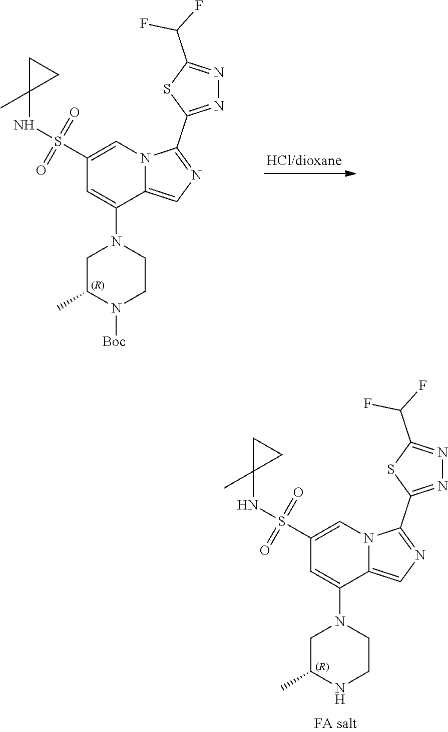

A mixture of tert-butyl (R)-4-(3-(5-(difluoromethyl)-1,3,4-thiadiazol-2-yl)-6-(N-(1-methylcyclopropyl)sulfamoyl)imidazo[1,5-a]pyridin-8-yl)-2-methylpiperazine-1-carboxylate (28 mg, 0.0480 mmol) in HCl/dioxane (1.0 mL, 4.00 mmol, 4N) was stirred at 25° C. for 1 h. and then concentrated under vacuum. The crude product was purified by preparative HPLC (column: Unisil 3-100 C18 Ultra 150*25 mm*10 μm; mobile phase: A: 0.225% formic acid in water, B: MeCN; B %: 12%-42%, 10 min) and lyophilized directly to give the product (R)-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide formate (1.4 mg, 0.00258 mmol, 5.38% yield, FA salt) as a yellow solid

RT 0.369 min (Method 3); m/z 484.2 (M+H)⁺ (ESI⁺); ¹H NMR (CDCl₃, 400 MHz): 9.87 (s, 1H), 8.30 (br, 1H), 7.70 (s, 1H), 7.08 (t, J=53.6 Hz, 1H), 6.69 (s, 1H), 5.35 (s, 1H), 3.69-3.61 (m, 2H), 3.32-3.27 (m, 2H), 3.12-3.06 (m, 2H), 2.81-2.76 (m, 1H), 1.38 (s, 3H), 1.29 (d, J=6.4 Hz, 3H), 0.95-0.92 (m, 2H), 0.61-0.58 (m, 2H).

### Preparation of Example 198

### (R)-3-(5-(difluoromethyl)-1,3,4-thiadiazol-2-yl)-8-(4-isobutyryl-3-methylpiperazin-1-yl)-N-(1-methylcyclopropyl)imidazo[1,5-a]pyridine-6-sulfonamide

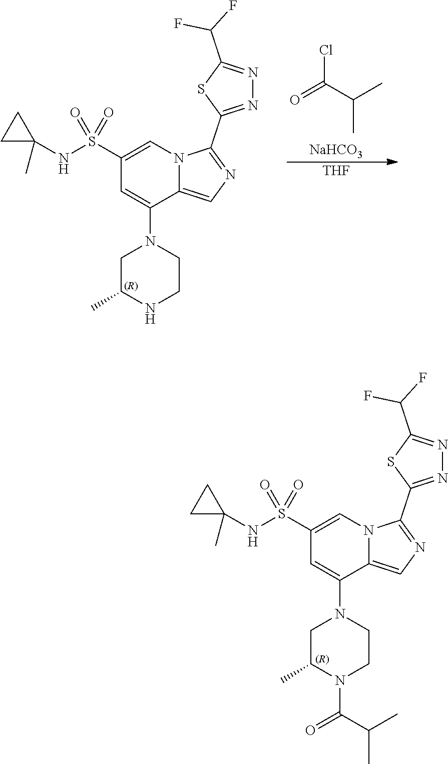

To a mixture of (R)-3-(5-(difluoromethyl)-1,3,4-thiadiazol-2-yl)-N-(1-methylcyclopropyl)-8-(3-methylpiperazin-1-yl)imidazo[1,5-a]pyridine-6-sulfonamide (22 mg, 0.0414 mmol) in THE (0.5 mL) and water (0.5 mL) was added 2-methylpropanoyl chloride (8.8 mg, 0.0827 mmol) and NaHCO₃ (3.5 mg, 0.0414 mmol) at 0° C. The reaction mixture was stirred at 0° C. for 15 min, then quenched with water (6 mL) and extracted with EtOAc (6 mL; 2×). The organic layers were separated, combined, dried over anhydrous Na₂SO₄ and concentrated undervacuum. The crude product was purified by preparative HPLC (column: Unisil 3-100 C18 Ultra 150*25 mm*10 μm; mobile phase: A: 0.225% formic acid in water, B: MeCN; B %: 45%-75%, 10 min) and lyophilized directly to give the product (R)-3-(5-(difluoromethyl)-1,3,4-thiadiazol-2-yl)-8-(4-isobutyryl-3-methylpiperazin-1-yl)-N-(1-methylcyclopropyl)imidazo[1,5-a]pyridine-6-sulfonamide (3.3 mg, 0.00578 mmol, 13.98% yield) as a yellow solid.

RT 0.483 min (method 1), m/z 554.4 (M+H)⁺ (ESI⁺), ¹H NMR (CDCl₃, 400 MHz) 9.88 (s, 1H), 7.74 (br, 1H), 7.08 (t, J=53.6 Hz, 1H), 6.65 (s, 1H), 5.09 (s, 1H), 5.00-4.46 (m, 1H), 4.39-3.88 (m, 1H), 3.68-3.61 (m, 2H), 3.02-2.97 (m, 2H), 2.96-2.86 (m, 1H), 1.49-1.48 (m, 1H), 1.39 (s, 3H), 1.27-1.05 (m, 9H), 0.97-0.89 (m, 2H), 0.58-0.63 (m, 2H).

### Preparation of Example 199

### 3-(5-(difluoromethyl)-1,3,4-thiadiazol-2-yl)-N-(1-(fluoromethyl)cyclopropyl)-8-(2-oxa-7-azaspiro[3.5]nonan-7-yl)imidazo[1,5-a]pyridine-6-sulfonamide

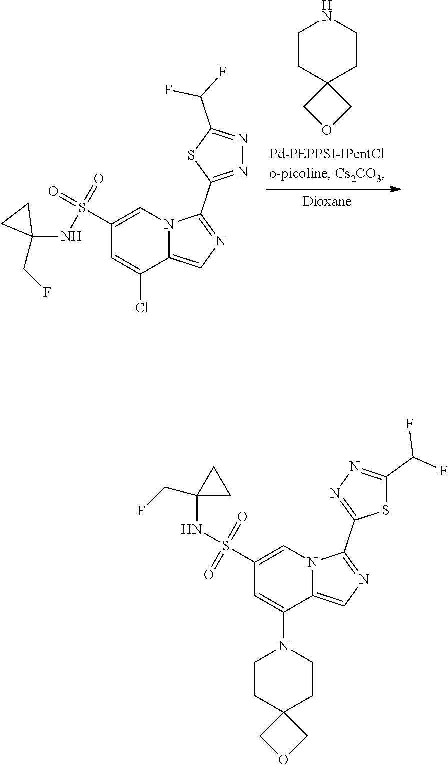 [SMILES: O=S(=O)(NC1(CF)CC1)c1cc(Cl)c2cnc(-c3nnc(C(F)F)s3)n2c1 ; C1CC2(CCN1)COC2 ; O=S(=O)(NC1(CF)CC1)c1cc(N2CCC3(CC2)COC3)c2cnc(-c3nnc(C(F)F)s3)n2c1]

To a mixture of 8-chloro-3-(5-(difluoromethyl)-1,3,4-thiadiazol-2-yl)-N-(1-(fluoromethyl)cyclopropyl)imidazo[1,5-a]pyridine-6-sulfonamide (30 mg, 0.0685 mmol) in dioxane (0.5 mL) were added 2-oxa-7-azaspiro[3.5]nonane (17 mg, 0.137 mmol), Pd-PEPPSI-IPentCl o-picoline (5.9 mg, 0.00685 mmol) and Cs₂CO₃ (67 mg, 0.206 mmol). The reaction mixture was degassed with N₂ (3×), stirred at 90° C. for 30 min and then concentrated under vacuum. The resulting residue was diluted with MeOH (3 mL), filtered and the filtrate was purificated by preparative HPLC (column: Phenomenex luna C18 150*25 mm*10 μm; mobile phase: A: 0.225% formic acid in water, B: MeCN; B %: 40%-70%, 10 min) and lyophilized directly to give the product 3-(5-(difluoromethyl)-1,3,4-thiadiazol-2-yl)-N-(1-(fluoromethyl)cyclopropyl)-8-(2-oxa-7-azaspiro[3.5]nonan-7-yl)imidazo[1,5-a]pyridine-6-sulfonamide (2.8 mg, 0.00518 mmol, 7.55% yield) as a yellow solid.

RT 0.458 min (method 1); m/z 529.3 (M+H)⁺ (ESI⁺), ¹H NMR (CDCl₃, 400 MHz) 9.85 (s, 1H), 7.70 (s, 1H), 7.08 (t, J=54.0 Hz, 1H), 6.61 (s, 1H), 5.47 (s, 1H), 4.54 (s, 4H), 4.26 (d, J=48.4 Hz, 2H), 3.27-3.24 (m, 4H), 2.16-2.14 (m, 4H), 1.18-1.14 (m, 2H), 0.88-0.85 (m, 2H).

### Preparation of Example 200 1-chloro-3-(5-(difluoromethyl)-1,3,4-thiadiazol-2-yl)-N-(1-(fluoromethyl)cyclopropyl)-8-(4-isobutyrylpiperazin-1-yl)imidazo[1,5-a]pyridine-6-sulfonamide

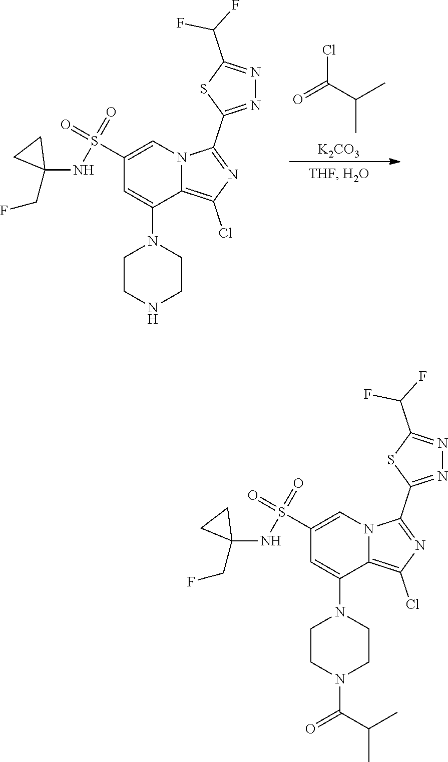

At 0° C., to a solution of 1-chloro-3-(5-(difluoromethyl)-1,3,4-thiadiazol-2-yl)-N-(1-(fluoromethyl)cyclopropyl)-8-(piperazin-1-yl)imidazo[1,5-a]pyridine-6-sulfonamide (15 mg, 28.74 μmol, FA sale) in THE (0.5 mL) and H₂O (0.25 mL) was added K₂CO₃ (11.92 mg, 86.21 μmol) followed by isobutyryl chloride (6.12 mg, 57.47 μmol, 6.00 μL). The mixture was stirred at 25° C. for 1 h then, diluted with brine (15 mL) and extracted with EtOAc (7 mL, 3×). The organic layers were separated, combined, dried over anhydrous Na₂SO₄, filtered and concentrated under vacuum. The crude product was purified by preparative HPLC (column: Phenomenex luna C18 150*25 mm*10 μm; mobile phase: A: 0.225% formic acid in water, B: MeCN; B %: 40%-70%, 10 min) and lyophilized to give the product 1-chloro-3-(5-(difluoromethyl)-1,3,4-thiadiazol-2-yl)-N-(1-(fluoromethyl)cyclopropyl)-8-(4-isobutyrylpiperazin-1-yl)imidazo[1,5-a]pyridine-6-sulfonamide (5.26 mg, 8.53 μmol, 29.68% yield, 96% purity) as a yellow solid.

RT 0.499 min (Method 3); m/z 592.3 (M+H)⁺ (ESI⁺); ¹H NMR (CDCl₃, 400 MHz): 9.92 (s, 1H), 7.09 (t, J=53.6 Hz, 1H), 6.73 (s, 1H), 5.52 (s, 1H), 4.27 (d, J=48.4 Hz, 2H), 3.65-3.41 (m, 4H), 3.27-3.17 (m, 4H), 2.88-2.81 (m, 1H), 1.20 (s, 3H), 1.19 (s, 3H), 1.18-1.14 (m, 2H), 0.91-0.82 (m, 2H).

Compounds listed in the table below were prepared according to the corresponding general procedures or when stated in a similar way to related compound and starting from the corresponding intermediates.

| Cpd | | Yield | | | |
|---|---|---|---|---|---|
| number | Structure | (%) | Procedure | LC/MS | 1H NMR |
| 201 | 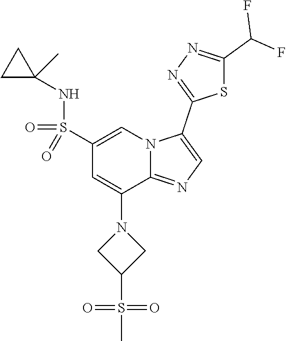 [SMILES: CC1(NS(=O)(=O)c2cc(N3CC(S(C)(=O)=O)C3)c3ncc(-c4nnc(C(F)F)s4)n3c2)CC1] | 7.30 | Procedure 1 | RT 0.848 min (method 1); m/z 519.1 (M + H)⁺ (ESI⁺) | ¹H NMR (CDCl₃, 400 MHz): 9.71 (d, J = 1.6 Hz, 1 H), 8.11 (s, 1 H), 7.10 (t, J = 53.6 Hz, 1 H), 6.50 (d, J = 1.6 Hz, 1 H), 5.05 (s, 1 H), 4.68-4.79 (m, 4 H), 4.18-4.29 (m, 1 H), 3.00 (s, 3 H), 1.37 (s, 3 H), 0.89-0.95 (m, 2 H), 0.56-0.62 (m, 2 H) |
| 202 | 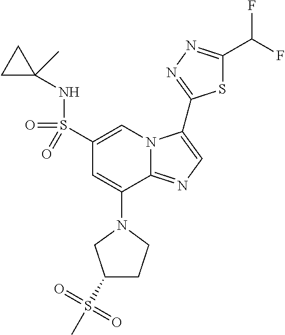 [SMILES: CC1(NS(=O)(=O)c2cc(N3CC[C@H](S(C)(=O)=O)C3)c3ncc(-c4nnc(C(F)F)s4)n3c2)CC1] | 13.14 | Procedure 1 | RT 0.890 min (method 1); m/z 532.9 (M + H)⁺ (ESI⁺) | ¹H NMR (DMSO-d₆, 400 MHz): 9.48 (s, 1 H), 8.58 (s, 1 H), 8.35 (s, 1 H), 7.69 (t, J = 53.6 Hz, 1 H), 6.63 (s, 1 H), 4.28- 4.31 (m, 1 H), 4.15- 4.18 (m, 2 H), 3.93- 3.96 (m, 1 H), 3.79- 3.82 (m, 1 H), 3.09 (s, 3 H), 2.54 (s, 2 H), 1.15 (s, 3 H), 0.68-0.74 (m, 2 H), 0.41-0.46 (m, 2 H) |
| 203 | 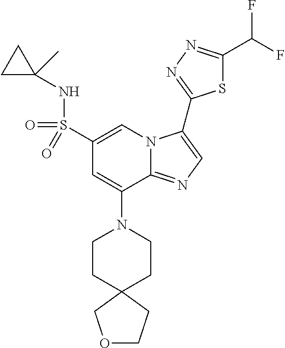 [SMILES: CC1(NS(=O)(=O)c2cc(N3CCC4(CCOC4)CC3)c3ncc(-c4nnc(C(F)F)s4)n3c2)CC1] | 8.90 | Procedure 1 | RT 0.926 min (method 1); m/z 524.9 (M + H)⁺ (ESI⁺) | ¹H NMR (DMSO-d₆, 400 MHz): 9.63 (d, J = 1.2 Hz, 1 H), 8.60 (s, 1 H), 8.39 (s, 1 H), 7.50-7.89 (m, 1 H), 7.00 (d, J = 1.2 Hz, 1 H), 3.80 (t, J = 7.2 Hz, 2 H), 3.66-3.75 (m, 2 H), 3.52-3.62 (m, 4 H), 1.81 (t, J = 7.2 Hz, 2 H), 1.74 (m, 4 H), 1.14 (s, 3 H), 0.66-0.75 (m, 2 H), 0.41-0.48 (m, 2 H) |
| 204 | 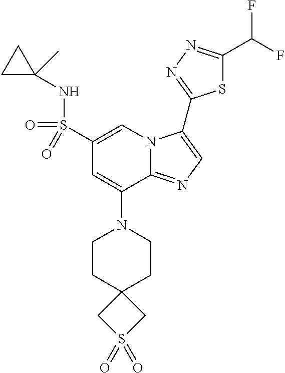 [SMILES: CC1(NS(=O)(=O)c2cc(N3CCC4(CC3)CS(=O)(=O)C4)c3ncc(-c4nnc(C(F)F)s4)n3c2)CC1] | 12.79 | Procedure 1 | RT 0.867 min (method 1); m/z 559.1 (M + H)⁺ (ESI⁺) | ¹H NMR (CDCl₃, 400 MHz): 9.91 (d, J = 1.6 Hz, 1 H), 8.18 (s, 1 H), 6.95-7.24 (m, 2 H), 5.10 (s, 1 H), 4.00 (s, 4 H), 3.57-3.68 (m, 4 H), 2.15-2.28 (m, 4 H), 1.37 (s, 3 H), 0.88- 0.95 (m, 2 H), 0.57- 0.63 (m, 2 H) |
| 205 | 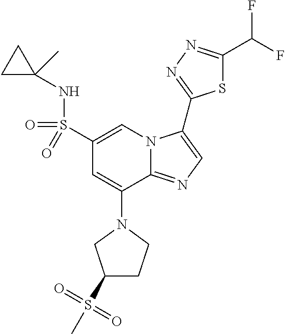 [SMILES: CC1(NS(=O)(=O)c2cc(N3CC[C@@H](S(C)(=O)=O)C3)c3ncc(-c4nnc(C(F)F)s4)n3c2)CC1] | 2.68 | Procedure 1 | RT 0.858 min (method 1); m/z 533.0 (M + H)⁺ (ESI⁺) | ¹H NMR (CDCl₃, 400 MHz): 9.72 (d, J = 1.2 Hz, 1 H), 8.11 (s, 1 H), 6.94-7.24 (m, 1 H), 6.63 (d, J = 1.2 Hz, 1 H), 5.13 (s, 1 H), 4.55 (m, 1 H), 4.38 (m, 1 H), 4.01- 4.11 (m, 1 H), 3.79- 3.93 (m, 2 H), 3.02 (s, 3 H), 2.48-2.70 (m, 2 H), 1.37 (s, 3 H), 0.90-0.95 (m, 2 H), 0.54-0.62 (m, 2 H) |
| 206 | 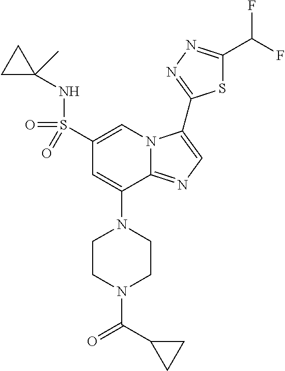 [SMILES: CC1(NS(=O)(=O)c2cc(N3CCN(C(=O)C4CC4)CC3)c3ncc(-c4nnc(C(F)F)s4)n3c2)CC1] | 12.2 | Procedure 1 | RT 0. 592 min (Method 5); m/z 538.4 (M + H)⁺ (ESI⁺) | ¹H NMR (CDCl₃, 400 MHz): 9.93 (d, J = 1.2 Hz, 1 H), 8.19 (s, 1 H), 6.96-7.24 (m, 2 H), 5.08 (s, 1 H), 3.90-4.05 (m, 4 H), 3.55-3.80 (m, 4 H), 1.77-1.84 (m, 1 H), 1.37 (s, 3 H), 1.03-1.10 (m, 2 H), 0.88-0.93 (m, 2 H), 0.82-0.87 (m, 2 H), 0.58-0.63 (m, 2 H) |
| 207 | 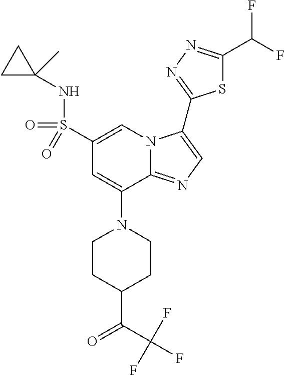 [SMILES: CC1(NS(=O)(=O)c2cc(N3CCC(C(=O)C(F)(F)F)CC3)c3ncc(-c4nnc(C(F)F)s4)n3c2)CC1] | 11.3 | Procedure 1 | RT 0.637 min (Method 5); m/z 566.3 (M + H)⁺ (ESI⁺) | ¹H NMR (CDCl₃, 400 MHz): 9.95 (d, J = 1.6 Hz, 1 H), 8.19 (s, 1 H), 6.94-7.25 (m, 2 H), 5.10 (s, 1 H), 3.97-4.02 (m, 2 H), 3.89-3.95 (m, 2 H), 3.68-3.78 (m, 4 H), 1.38 (s, 3 H), 0.88-0.93 (m, 2 H), 0.56-0.62 (m, 2 H) |
| 208 | 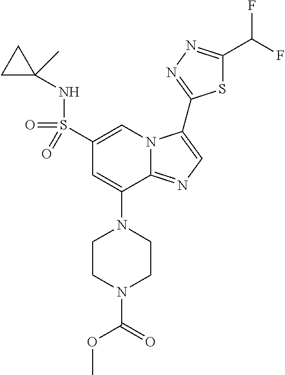 [SMILES: COC(=O)N1CCN(c2cc(S(=O)(=O)NC3(C)CC3)cn3c(-c4nnc(C(F)F)s4)cnc23)CC1] | 7.9 | Procedure 1 | RT 0.612 min (Method 5); m/z 528.3 (M + H)⁺ (ESI⁺) | ¹H NMR (CDCl₃, 400 MHz): 9.92 (d, J = 1.6 Hz, 1 H), 8.18 (s, 1 H), 6.93-7.24 (m, 2 H), 5.06 (s, 1 H), 3.77 (s, 7 H), 3.56-3.69 (m, 4 H), 1.37 (s, 3 H), 0.88- 0.94 (m, 2 H), 0.55- 0.63 (m, 2 H) |
| 209 | 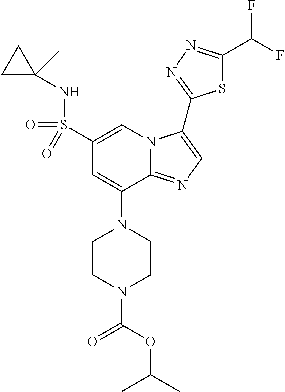 [SMILES: CC(C)OC(=O)N1CCN(c2cc(S(=O)(=O)NC3(C)CC3)cn3c(-c4nnc(C(F)F)s4)cnc23)CC1] | 18.1 | Procedure 1 | RT 0.647 min (Method 5); m/z 556.4 (M + H)⁺ (ESI⁺) | ¹H NMR (CDCl₃, 400 MHz): 9.91 (d, J = 1.6 Hz, 1 H), 8.18 (s, 1 H), 6.94-7.24 (m, 2 H), 5.07 (s, 1 H), 4.99 (m, 1 H), 3.71-3.81 (m, 4 H), 3.62 (m, 4 H), 1.36 (s, 3 H), 1.30 (d, J = 6.4 Hz, 6 H), 0.90- 0.93 (m, 2 H), 0.55- 0.62 (m, 2 H) |
| 210 | 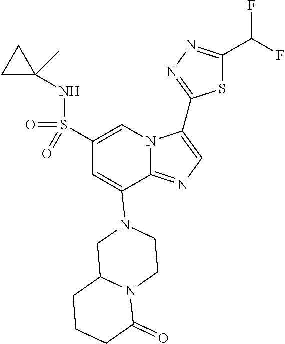 [SMILES: CC1(NS(=O)(=O)c2cc(N3CCN4C(=O)CCCC4C3)c3ncc(-c4nnc(C(F)F)s4)n3c2)CC1] | 17.9 | Procedure 1 | RT 0.586 min (Method 5); m/z 538.3 (M + H)⁺ (ESI⁺) | ¹H NMR (CDCl₃, 400 MHz): 9.92 (d, J = 1.2 Hz, 1 H), 8.18 (s, 1 H), 6.95-7.25 (m, 2 H), 5.18 (s, 1 H), 4.83-4.90 (m, 1 H), 4.40-4.47 (m, 1 H), 4.24-4.31 (m, 1 H), 3.69-3.84 (m, 1 H), 3.05 (m, 1 H), 2.87-2.98 (m, 1 H), 2.68 (t, J = 11.2 Hz, 1 H), 2.50-2.59 (m, 1 H), 2.37-2.48 (m, 1 H), 2.09-2.17 (m, 1 H), 1.89-2.01 (m, 1 H), 1.76-1.86 (m, 1 H), 1.62-1.68 (m, 1 H), 1.37 (s, 3 H), 0.87-0.94 (m, 2 H), 0.58-0.62 (m, 2 H) |
| 211 | 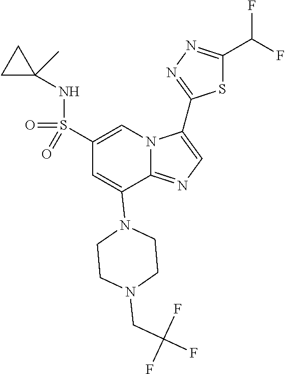 [SMILES: CC1(NS(=O)(=O)c2cc(N3CCN(CC(F)(F)F)CC3)c3ncc(-c4nnc(C(F)F)s4)n3c2)CC1] | 17.7 | Procedure 1 | RT 0.559 min (Method 5); m/z 552.2 (M + H)⁺ (ESI⁺) | ¹H NMR (CDCl₃, 400 MHz): 9.90 (d, J = 1.6 Hz, 1 H), 8.17 (s, 1 H), 6.94-7.24 (m, 2 H), 5.09 (s, 1 H), 3.64-3.73 (m, 4 H), 3.05-3.16 (m, 2 H), 2.92-3.02 (m, 4 H), 1.36 (s, 3 H), 0.86-0.96 (m, 2 H), 0.55-0.63 (m, 2 H) |
| 212 | 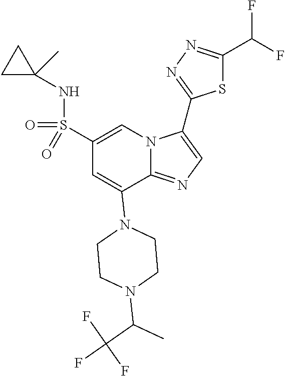 [SMILES: CC(N1CCN(c2cc(S(=O)(=O)NC3(C)CC3)cn3c(-c4nnc(C(F)F)s4)cnc23)CC1)C(F)(F)F] | 8.1 | Procedure 1 | RT 0.984 min (method 1); m/z 566.2 (M + H)⁺ (ESI⁺) | ¹H NMR (CDCl₃, 400 MHz): 9.89 (d, J = 1.2 Hz, 1 H), 8.17 (s, 1 H), 6.92- 7.25 (m, 2 H), 5.07 (s, 1 H), 3.64 (m, 4 H), 3.18-3.32 (m, 1 H), 2.91-3.15 (m, 4 H), 1.35 (s, 3 H), 1.32 (d, J = 7.2 Hz, 3 H), 0.87-0.95 (m, 2 H), 0.53-0.61 (m, 2 H) |
| 213 | 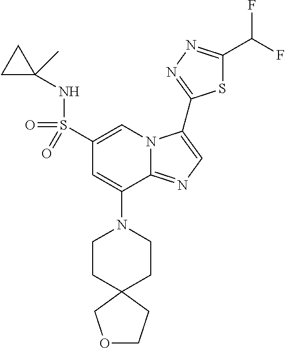 | 2.1 | Procedure 1 | RT 0.543 min (Method 4); m/z 562.1 (M + H)⁺ (ESI⁺) | ¹H NMR (CDCl₃, 400 MHz): 10.08 (s, 1 H), 7.32 (s, 1 H), 7.06 (t, J = 53.6 Hz, 1 H), 6.78 (s, 1 H), 5.50 (s, 1 H), 4.53 (s, 4 H), 4.26 (t, J = 48.8 Hz, 2 H), 2.51- 3.58 (m, 4 H), 2.00- 2.29 (m, 4 H), 1.12- 1.19 (m, 2 H), 0.83- 0.87 (m, 2 H) |
| 214 | 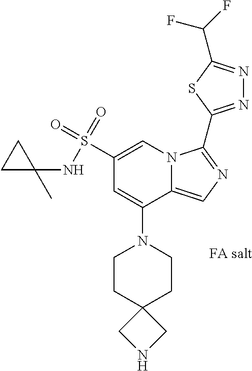 [SMILES: CC1(NS(=O)(=O)c2cc(N3CCC4(CC3)CNC4)c3cnc(-c4nnc(C(F)F)s4)n3c2)CC1] | 24.3 | Procedure 1 | RT 0.307 min (Method 4); m/z 510.1 (M + H)⁺ (ESI⁺) | ¹H NMR (DMSO-d₆, 400 MHz): 9.57 (s, 1 H), 8.41 (s, 1 H), 7.96 (s, 1 H), 7.37 (t, J = 53.6 Hz, 1 H), 6.68 (s, 1 H), 3.62 (s, 4 H), 3.23-3.26 (m, 4 H), 1.92-2.03 (m, 4 H), 1.15 (s, 3 H), 0.66- 0.76 (m, 2 H), 0.40- 0.48 (m, 2 H) |
| 215 | 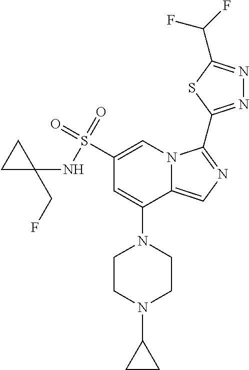 [SMILES: O=S(=O)(NC1(CF)CC1)c1cc(N2CCN(C3CC3)CC2)c2cnc(-c3nnc(C(F)F)s3)n2c1] | 13.24 | Procedure 1 | RT 0.351 min (Method 4); m/z 528.4 (M + H)⁺ (ESI⁺) | ¹H NMR (CDCl₃, 400 MHz): 9.86 (s, 1H), 7.73 (s, 1H), 7.08 (t, J = 53.6 Hz, 1H), 6.63 (s, 1H), 5.55 (s, 1H), 4.27 (d, J = 48.4 Hz, 2H), 3.36 (s, 4H), 2.93 (s, 4H), 1.83-1.78 (m, 1H), 1.18-1.12 (m, 2H), 0.89-0.84 (m, 2H), 0.55-0.57 (m, 4H). |
| 216 | 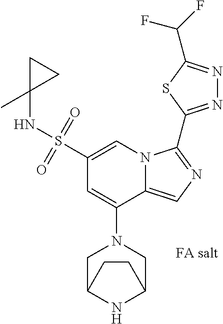 | 14.52 (yield after 2 steps) | Procedure 1 then Procedure 2 | RT 0.333 min (Method 4); m/z 496.2 (M + H)⁺ (ESI⁺) | ¹H NMR (DMSO-d₆, 400 MHz): 9.56 (s, 1H), 8.42 (s, 1H), 8.21 (s, 1H), 8.03 (s, 1H), 7.67 (t, J = 53.2 Hz, 1H), 6.57 (s, 1H), 3.67-3.63 (m, 2H), 3.5 (d, J = 10.8 Hz, 2H), 3.07 (d, J = 11.6 Hz, 2H), 1.96-1.89 (m, 2H), 1.83-1.76 (m, 2H), 1.16 (s, 3H), 0.75-0.69 (m, 2H), 0.48-0.41 (m, 2H). |
| 217 | 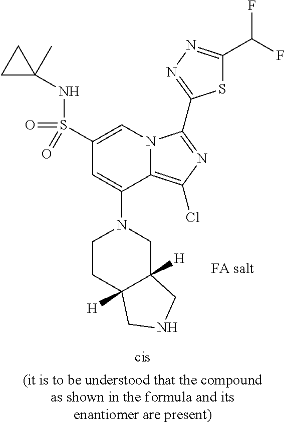 [SMILES: CC1(NS(=O)(=O)c2cc(N3CC[C@H]4CNC[C@H]4C3)c3c(Cl)nc(-c4nnc(C(F)F)s4)n3c2)CC1] | 6.82 (yield after 2 steps) | Procedure 1 then Procedure 2 | RT 362 min (Method 4); m/z 544.2 (M + H)⁺ (ESI⁺) | ¹H NMR (DMSO-d₆, 400 MHz): 9.61 (s, 1H), 9.00-8.51 (m, 1H), 8.39 (s, 1H), 7.69 (t, J = 52.8 Hz, 1H), 6.87 (s, 1H), 3.25-3.20 (m, 4H), 3.13-3.07 (m, 2H), 3.06-3.03 (m, 1H), 2.86-2.79 (m, 1H), 2.63-2.57 (m, 1H), 2.45-2.37 (m, 1H), 2.04-1.76 (m, 2H), 1.17 (s, 3H), 0.81-0.69 (m, 2H), 0.53-0.40 (m, 2H). |
| 218 | 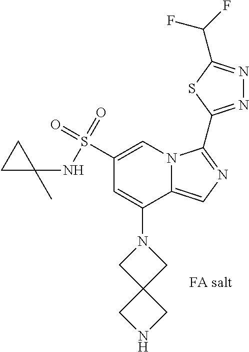 [SMILES: CC1(NS(=O)(=O)c2cc(N3CC4(CNC4)C3)c3cnc(-c4nnc(C(F)F)s4)n3c2)CC1] | 37.92 (yield after 2 steps) | Procedure 1 then Procedure 2 | RT 330 min (Method 4); m/z 482.0 (M + H)⁺ (ESI⁺) | ¹H NMR (DMSO-d₆, 400 MHz): 9.37 (s, 1H), 8.68-8.13 (m, 1H), 8.31 (s, 1H), 7.91 (s, 1H), 7.69 (t, J = 52.8 Hz, 1H), 6.05 (s, 1H), 4.40 (s, 4H), 4.01 (s, 4H), 1.17 (s, 3H), 0.78- 0.64 (m, 2H), 0.49- 0.37 (m, 2H). |
| 219 | 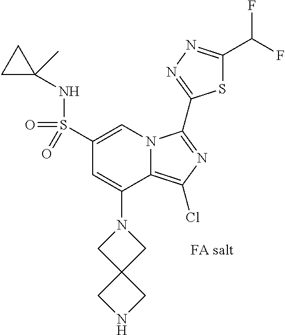 | 10.31 (yield after 2 steps) | Procedure 1 then Procedure 2 | RT 0.362 min (Method 4); m/z 516.2 (M + H)⁺ (ESI⁺) | ¹H NMR (DMSO-d₆, 400 MHz): 9.49 (s, 1H), 9.01-8.31 (m, 1H), 8.30 (s, 1H), 7.68 (t, J = 53.2 Hz, 1H), 6.29 (s, 1H), 4.35 (s, 4H), 4.04 (s, 4H), 1.18 (s, 3H), 0.80-0.66 (m, 2H), 0.52-0.39 (m, 2H). |
| 220 | 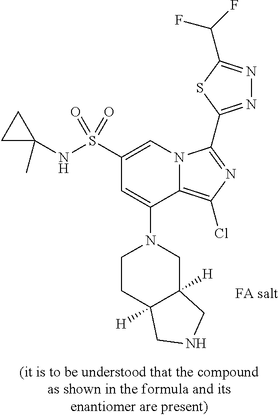 [SMILES: CC1(NS(=O)(=O)c2cc(N3CC[C@@H]4CNC[C@@H]4C3)c3c(Cl)nc(-c4nnc(C(F)F)s4)n3c2)CC1] | 10.29 (yield after 2 steps) | Procedure 1 then Procedure 2 | RT 0.355 min (Method 4); m/z 544.2 (M + H)⁺ (ESI⁺) | ¹H NMR (DMSO-d₆, 400 MHz): 9.61 (s, 1H), 9.00-8.33 (m, 1H), 8.34 (s, 1H), 7.69 (t, J = 53.2 Hz, 1H), 6.87 (s, 1H), 3.25-3.21 (m, 4H), 3.12-3.06 (m, 2H), 2.98-2.91 (m, 1H), 2.87-2.78 (m, 1H), 2.63-2.57 (m, 1H), 2.45-2.37 (m, 1H), 1.99-1.83 (m, 2H), 1.17 (s, 3H), 0.79-0.69 (m, 2H), 0.53-0.40 (m, 2H). |
| 221 | 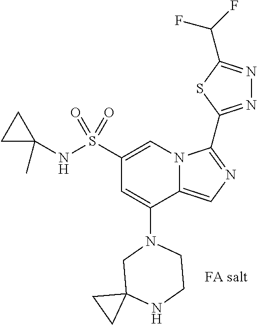 [SMILES: CC1(NS(=O)(=O)c2cc(N3CCNC4(CC4)C3)c3cnc(-c4nnc(C(F)F)s4)n3c2)CC1] | 16.17 (yield after 2 steps) | Procedure 1 then Procedure 2 | RT 0.324 min (Method 4); m/z 496.3 (M + H)⁺ (ESI⁺) | ¹H NMR (DMSO-d₆, 400 MHz): 9.57 (s, 1H), 8.42 (s, 1H), 8.15 (s, 1H), 7.89 (s, 1H), 7.67 (t, J = 53.2 Hz, 1H), 6.65 (s, 1H), 3.28-3.26 (m, 2H), 3.17-3.14 (m, 2H), 3.05-3.00 (m, 2H), 1.15 (s, 3H), 0.77- 0.69 (m, 2H), 0.66- 0.58 (m, 2H), 0.57- 0.52 (m, 2H), 0.48- 0.42 (m, 2H). |
| 222 | 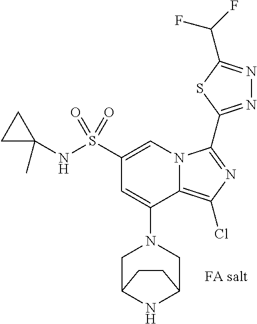 [SMILES: CC1(NS(=O)(=O)c2cc(N3CC4CCC(C3)N4)c3c(Cl)nc(-c4nnc(C(F)F)s4)n3c2)CC1] | 8.19 (yield after 2 steps) | Procedure 1 then Procedure 2 | RT 0.359 min (Method 4); m/z 530.2 (M + H)⁺ (ESI⁺) | ¹H NMR (DMSO-d₆, 400 MHz): 9.64 (s, 1H), 8.54 (s, 1H), 8.22 (d, J = 2.4 Hz, 1H), 7.67 (t, J = 53.2 Hz, 1H), 6.87 (s, 1H), 3.68-3.63 (m, 2H), 3.30-3.29 (m, 2H), 2.99-2.94 (m, 2H), 2.21-2.15 (m, 2H), 1.80-1.73 (m, 2H), 1.17 (s, 3H), 0.76- 0.70 (m, 2H), 0.49- 0.43 (m, 2H). |
| 223 | 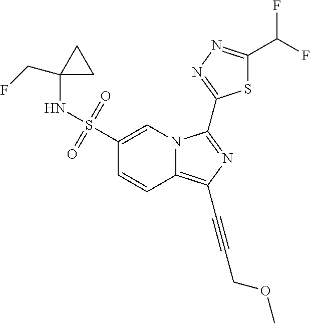 [SMILES: COCC#Cc1nc(-c2nnc(C(F)F)s2)n2cc(S(=O)(=O)NC3(CF)CC3)ccc12] | 10.17 | Procedure 10 | RT 0.470 min (Method 4); m/z 472.2 (M + H)⁺ (ESI⁺) | ¹H NMR (CDCl₃, 400 MHz) 10.20 (s, 1H), 7.88 (d, J = 9.6 Hz, 1H), 7.45 (dd, J = 1.6, 9.6 Hz, 1H), 7.09 (t, J = 53.6 Hz, 1H), 5.58 (s, 1H), 4.46 (s, 2H), 4.28 (d, J = 48.4 Hz, 2H), 3.52 (s, 3H), 1.18-1.09 (m, 2H), 0.92-0.84 (m, 2H) |
| 224 | 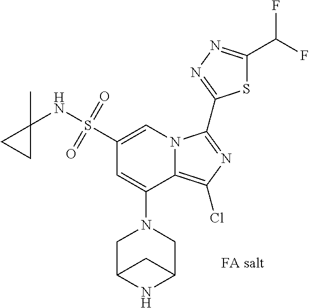 | 10.82 (yield after 2 steps) | Procedure 1 then Procedure 2 | RT 0.378 min (Method 4); m/z 515.9 (M + H)⁺ (ESI⁺) | ¹H NMR (DMSO-d₆, 400 MHz) 9.62 (s, 1H), 8.56 (s, 1H), 8.29 (s, 1H), 7.69 (t, J = 53.2 Hz, 1H), 7.07 (s, 1H), 3.94-3.88 (m, 2H), 3.71 (d, J = 10.4 Hz, 2H), 3.59 (d, J = 11.2 Hz, 2H), 2.64- 2.61 (m, 1H), 2.20 (d, J = 8.8 Hz, 1H), 1.20 (s, 3H), 0.77-0.71 (m, 2H), 0.49-0.46 (m, 2H) |
| 225 | 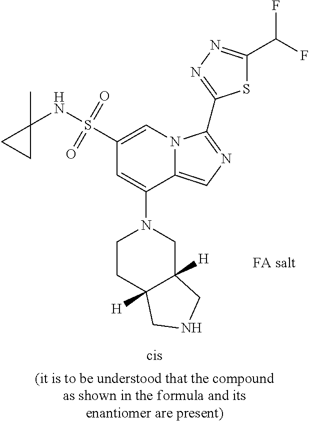 [SMILES: CC1(NS(=O)(=O)c2cc(N3CC[C@H]4CNC[C@H]4C3)c3cnc(-c4nnc(C(F)F)s4)n3c2)CC1 ; "FA salt"] | 35.49 (yield after 2 steps) | Procedure 1 then Procedure 2 | RT 0.364 min (Method 4); m/z 510.1 (M + H)⁺ (ESI⁺) | ¹H NMR (DMSO-d₆, 400 MHz) 9.56 (s, 1H), 8.43 (s, 1H), 8.39 (s, 1H) 7.91 (s, 1H), 7.68 (t, J = 53.2 Hz, 1H), 6.66 (s, 1H), 3.45-3.39 (m, 2H), 3.38-3.28 (m, 2H), 3.25-3.16 (m, 2H), 3.12-3.02 (m, 2H), 2.62-2.56 (m, 1H), 2.46-2.42 (m, 1H), 1.98-1.88 (m, 1H), 1.87-1.74 (m, 1H), 1.16 (s, 3H), 0.78- 0.66 (m, 2H), 0.50- 0.41 (m, 2H) |
| 226 | 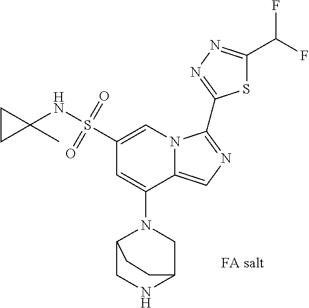 [SMILES: CC1(NS(=O)(=O)c2cc(N3CC4CCC3CN4)c3cnc(-c4nnc(C(F)F)s4)n3c2)CC1 ; "FA salt"] | 6.09 (yield after 2 steps) | Procedure 1 then Procedure 2 | RT 0.358 min (Method 4); m/z 496.3 (M + H)⁺ (ESI⁺) | ¹H NMR (DMSO-d₆, 400 MHz) 9.43 (s, 1H), 8.36 (s, 1H), 8.26 (s, 1H), 8.06 (s, 1H), 7.67 (t, J = 53.2 Hz, 1H), 6.36 (s, 1H), 4.15-4.06 (m, 1H), 3.98-3.80 (m, 1H), 3.78-3.71 (m, 1H), 3.40-3.29 (m, 2H), 3.23-3.10 (m, 1H), 2.22-2.07 (m, 1H), 2.03-1.87 (m, 2H), 1.85-1.75 (m, 1H), 1.17 (s, 3H), 0.79-0.69 (m, 2H), 0.51-0.38 (m, 2H) |
| 227 | 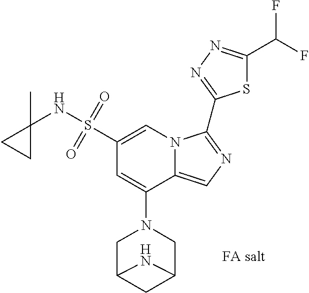 [SMILES: CC1(NS(=O)(=O)c2cc(N3CC4CC(C3)N4)c3cnc(-c4nnc(C(F)F)s4)n3c2)CC1 ; "FA salt"] | 13.19 (yield after 2 steps) | Procedure 1 then Procedure 2 | RT 0.352 min (Method 4); m/z 482.0 (M + H)⁺ (ESI⁺) | ¹H NMR (DMSO-d₆, 400 MHz) 9.47 (s, 1H), 8.38 (s, 1H), 8.32 (s, 1H), 8.23 (s, 1H), 7.67 (t, J = 53.2 Hz, 1H), 6.30 (s, 1H), 3.96-3.90 (m, 4H), 3.85 (d, J = 6.0 Hz, 2H), 2.65-2.55 (m, 1H), 1.62 (d, J = 8.8 Hz, 1H), 1.20 (s, 3H), 0.78-0.68 (m, 2H), 0.51-0.40 (m, 2H) |
| 228 | 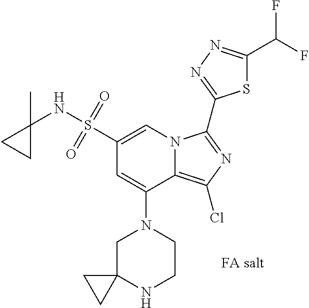 | 8.66 (yield after 2 steps) | Procedure 1 then Procedure 2 | RT 0.369 min (Method 4); m/z 529.9 (M + H)⁺ (ESI⁺) | ¹H NMR (DMSO-d₆, 400 MHz) 9.59 (s, 1H), 8.51 (s, 1H), 8.20 (s, 1H), 7.69 (t, J = 53.2 Hz, 1H), 6.76 (s, 1H), 3.15- 3.05 (m, 2H), 3.05- 2.98 (m, 2H), 2.97- 2.90 (m, 2H), 1.17 (s, 3H), 0.80-0.70 (m, 2H), 0.56-0.50 (m, 4H), 0.48-0.43 (m, 2H) |
| 229 | 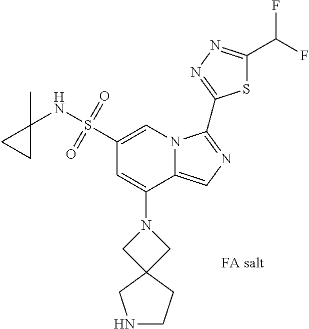 [SMILES: CC1(NS(=O)(=O)c2cc(N3CC4(CCNC4)C3)c3cnc(-c4nnc(C(F)F)s4)n3c2)CC1 ; "FA salt"] | 5.99 (yield after 2 steps) | Procedure 1 then Procedure 2 | RT 0.337 min (Method 4); m/z 496.0 (M + H)⁺ (ESI⁺) | ¹H NMR (DMSO-d₆, 400 MHz) 9.38 (s, 1H), 8.33 (s, 1H), 8.30 (s, 1H), 7.89 (s, 1H), 7.68 (t, J = 53.6 Hz, 1H), 6.06 (s, 1H), 4.26-4.21 (m, 4H), 3.23 (s, 2H), 3.04 (t, J = 6.8 Hz, 2H), 2.15 (t, J = 6.8 Hz, 2H), 1.19 (s, 3H), 0.77-0.69 (m, 2H), 0.49-0.41 (m, 2H) |
| 230 | 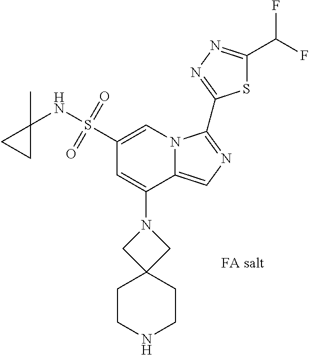 [SMILES: CC1(NS(=O)(=O)c2cc(N3CC4(CCNCC4)C3)c3cnc(-c4nnc(C(F)F)s4)n3c2)CC1 ; "FA salt"] | 3.63 (yield after 2 steps) | Procedure 1 then Procedure 2 | RT 0.353 min (Method 4); m/z 510.0 (M + H)⁺ (ESI⁺) | ¹H NMR (DMSO-d₆, 400 MHz) 9.35 (s, 1H), 8.36 (s, 1H), 8.29 (s, 1H), 7.94 (s, 1H), 7.67 (t, J = 53.2 Hz, 1H), 6.03 (s, 1H), 4.02 (s, 4H), 2.90-2.80 (m, 4H), 1.86-1.78 (m, 4H), 1.18 (s, 3H), 0.74- 0.69 (m, 2H), 0.47- 0.40 (m, 2H) |
| 231 | 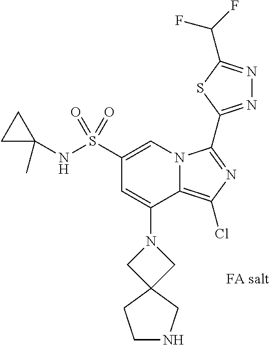 | 3.05 (yield after 2 steps) | Procedure 1 then Procedure 2 | RT 0.394 min (Method 4); m/z 530.0 (M + H)⁺ (ESI⁺) | ¹H NMR (D₂O, 400 MHz) 9.31 (s, 1H), 8.42 (d, J = 0.8 Hz, 1H), 7.32 (t, J = 54.0 Hz, 1H), 6.29 (s, 1H), 4.31-4.24 (m, 4H), 3.62 (s, 2H), 3.44 (t, J = 7.2 Hz, 2H), 2.43 (t, J = 7.2 Hz, 2H), 1.15 (s, 3H), 0.78-0.71 (m, 2H), 0.52-0.45(m, 2H) |
| 232 | 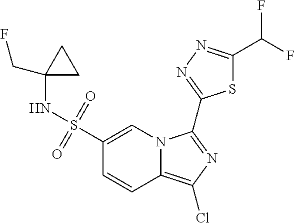 [SMILES: O=S(=O)(NC1(CF)CC1)c1ccc2c(Cl)nc(-c3nnc(C(F)F)s3)n2c1] | 24.77 | Procedure 3 | RT 0.467 min (method 1); m/z 438.1 (M + H)⁺ (ESI⁺) | ¹H NMR (CDCl₃, 400 MHz): 10.16 (s, 1H), 7.76 (d, J = 9.6 Hz, 1H)), 7.39 (d, J = 9.6 Hz, 1H), 7.09 (t, J = 53.6 Hz, 1H), 5.58 (s, 1H), 4.29 (d, J = 48.4 Hz, 2H), 1.13 (d, J = 4.6 Hz, 2H), 0.89 (t, J = 6.4 Hz, 2H). |
| 233 | 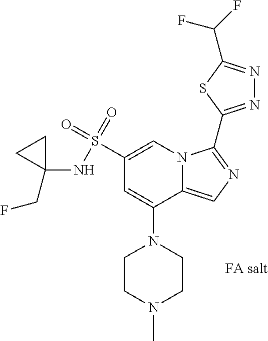 [SMILES: CN1CCN(c2cc(S(=O)(=O)NC3(CF)CC3)cn3c(-c4nnc(C(F)F)s4)ncc23)CC1] | 18.86 | Procedure 1 | RT 0.348 min (method 1); m/z 502.3 (M + H)⁺ (ESI⁺) | ¹H NMR (CDCl₃, 400 MHz): 9.87 (s, 1H), 8.25 (s, 1H), 7.70 (s, 1 H), 7.08 (t, J = 53.6 Hz, 1H), 6.68 (s, 1H), 5.90 (s, 1H), 4.27 (d, J = 48.8 Hz, 2H), 3.44 (s, 4H), 2.83 (s, 4H), 2.50 (s, 3H), 1.12- 1.18 (m, 2H), 0.86 (t, J = 6.0 Hz, 2H). |
| 234 | 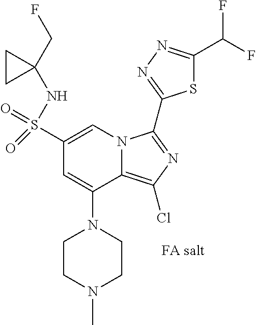 | 9.43 | Procedure 1 | RT 0.368 min (method 1); m/z 536.2 (M + H)⁺ (ESI⁺) | ¹H NMR (CDCl₃, 400 MHz): 9.90 (s, 1H), 8.28 (s, 1H), 7.08 (t, J = 53.6 Hz, 1H), 6.82 (s, 1H), 6.16-5.93 (m, 1H), 4.27 (d, J = 48.8 Hz, 2H), 3.32 (s, 4H), 2.96-2.92 (m, 4H), 2.55 (s, 3H), 1.19- 1.09 (m, 2H), 0.87 (s, 2H). |
| 235 | 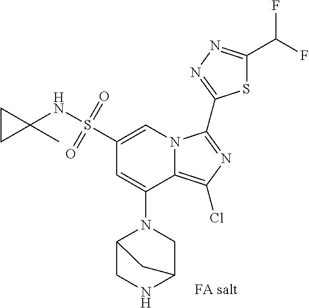 | 2.57 (yield after 2 steps) | Procedure 1 then Procedure 2 | RT 0.376 min (method 1); m/z 516.1 (M +H)+ (ESI⁺) | ¹H NMR (DMSO-d₆, 400MHz): 9.53 (s, 1H), 8.59-8.39 (m, 1H), 8.23 (s, 1H), 7.69 (t, J = 53.6 Hz, 1H), 6.78 (s, 1H), 4.45 (s, 1H), 4.02 (s, 1H), 3.64 (s, 1H), 3.51 (s, 1H), 3.15 (s, 1H), 3.04 (s, 1H), 1.93 (d, J = 9.2 Hz, 1H), 1.80 (d, J = 9.6 Hz, 1H), 1.18 (s, 3H), 0.77-0.70 (m, 2H), 0.48-0.42 (m, 2H). |
| 236 | 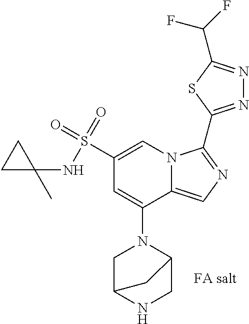 [SMILES: CC1(NS(=O)(=O)c2cc(N3CC4CC3CN4)c3cnc(-c4nnc(C(F)F)s4)n3c2)CC1] | 5.09 (yield after 2 steps) | Procedure 1 then Procedure 2 | RT 0.353 min (method 1); m/z 482.1 (M + H)⁺ (ESI⁺) | ¹H NMR (DMSO-d₆, 400 MHz): 9.39 (s, 1H), 8.36 (s, 1H), 8.24 (s, 1H), 8.07 (s, 1H), 7.67 (t, J = 53.6 Hz, 1H), 6.26 (s, 1H), 4.56 (s, 1H), 4.02 (d, J = 9.2 Hz, 1H), 3.85 (s, 1H), 3.56-3.54 (m, 1H), 3.13-3.02 (m, 2H), 1.99 (d, J = 9.2 Hz, 1H), 1.83 (d, J = 9.6 Hz, 1H), 1.18 (s, 3H), 0.73 (s, 2H), 0.45 (s, 2H). |
| 237 | 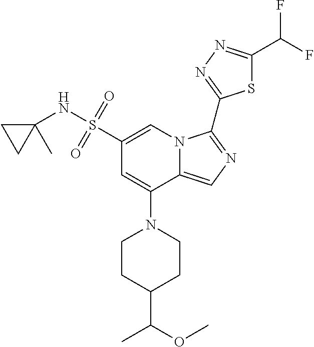 [SMILES: COC(C)C1CCN(c2cc(S(=O)(=O)NC3(C)CC3)cn3c(-c4nnc(C(F)F)s4)ncc23)CC1] | 16.73 | Procedure 1 | RT 0.504 min; (Method 4); m/z 527.3 (M + H)⁺ (ESI⁺) | ¹H NMR (CDCl₃ 400 MHz) 9.82 (s, 1H), 7.71 (s, 1H), 7.07((t, J = 53.6 Hz, 1H), 6.63 (s, 1H), 5.15 (s, 1H), 3.82 (d, J = 12.0 Hz, 2H), 3.38 (s, 3H), 3.25-3.15 (m, 1H), 2.92-2.80 (m, 2H), 2.04 (d, J = 11.2 Hz, 1H), 1.86-1.80 (m, 1H), 1.38 (s, 3H), 1.33- 1.27 (m, 2H), 1.21- 1.25 (m, 1H), 1.19 (d, J = 6.0 Hz, 3H), 0.98-0.91 (m, 2H), 0.61-0.56 (m, 2H) |
| 238 | 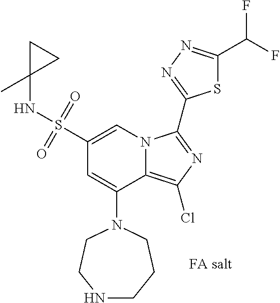 | 13.26 (yield after 2 steps) | Procedure 1 then Procedure 2 | RT 0.364 min; (Method 4); m/z 518.4 (M + H)⁺ (ESI⁺) | ¹H NMR (DMSO-d₆, 400 MHz): 9.58 (s, 1H), 8.34 (s, 1H), 7.67 (t, J = 53.2 Hz, 1H), 6.89 (s, 1H), 3.43-3.42 (m, 2H), 3.40-3.39 (m, 2H), 3.18 (d, J = 2.8 Hz, 2H), 3.12 (s, 2H), 2.01 (s, 2H), 1.16(s, 3H), 0.80-0.63 (m, 2H), 0.51-0.35 (m, 2H) |
| 239 | 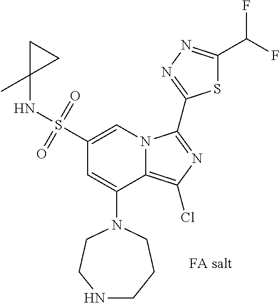 | 20.29 (yield after 2 steps) | Procedure 1 then Procedure 2 | RT 0.337 min; (Method 4); m/z 484.1 (M + H)⁺ (ESI⁺) | ¹H NMR (DMSO-d₆, 400 MHz) 9.46 (s, 1H), 8.50-8.34 (m, 1H), 8.26 (s, 1H), 8.01 (s, 1H), 7.67 ((t, J = 53.2 Hz, 1H), 6.39 (s, 1H), 3.79- 3.77 (m, 2H), 3.74- 3.75 (m, 2H), 3.19 (s, 2H), 2.96 (d, J = 6.0 Hz, 2H), 2.02 (s, 2H), 1.16 (s, 3H), 0.80-0.63 (m, 2H), 0.51-0.35 (m, 2H) |
| 240 | 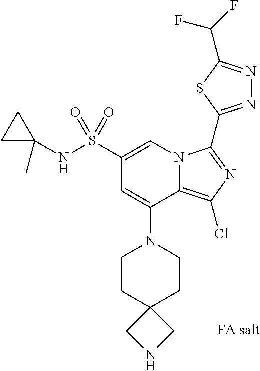 [SMILES: CC1(NS(=O)(=O)c2cc(N3CCC4(CC3)CNC4)c3c(Cl)nc(-c4nnc(C(F)F)s4)n3c2)CC1] | 10.01 (yield after 2 steps) | Procedure 1 then Procedure 2 | RT 0.382 min; (Method 4); m/z 544.1 (M + H)⁺ (ESI⁺) | ¹H NMR (DMSO-d₆, 400 MHz) 9.60 (s, 1H), 8.41 (s, 1H), 7.68 ((t, J = 53.2 Hz, 1H), 6.95 (s, 1H), 3.65 (s, 4H), 3.02 (d, J = 2.8 Hz, 4H), 2.02 (s, 4H), 1.16 (s, 3H), 0.80-0.64 (m, 2H), 0.53-0.40 (m, 2H) |
| 241 | 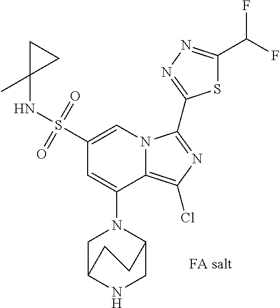 | 8.49 (yield after 2 steps) | Procedure 1 then Procedure 2 | RT 0.377 min; (Method 4); m/z 530.1 (M + H)⁺ (ESI⁺) | ¹H NMR (DMSO-d₆, 400 MHz) 9.56 (s, 1H), 8.55-8.50 (m, 1H), 8.28 (s, 1H), 7.68( t, J = 53.2 Hz, 1H), 6.96 (s, 1H), 3.74-3.71 (m, 1H), 3.58-3.55 (m, 1H), 3.38-3.35 (m, 1H), 3.22-3.20 (m, 2H), 3.10-3.08 (m, 1H), 2.19-2.17 (m, 1H), 1.96-1.94 (m, 1H), 1.88-1.78 (m, 2H), 1.78 (s, 3H), 0.75- 0.74 (m, 2H), 0.48- 0.45 (m, 2H), |
| 242 | 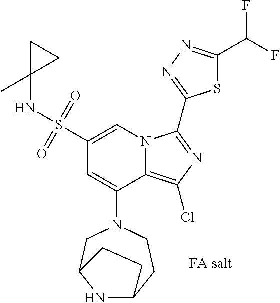 | 9.33 (yield after 2 steps) | Procedure 1 then Procedure 2 | RT 0.385 min; (Method 4); m/z 544.1 (M + H)⁺ (ESI⁺) | ¹H NMR (DMSO-d₆, 400 MHz) 9.60 (s, 1H), 8.55 (s, 1H), 8.20 (s, 1H), 7.68(t, J = 53.2 Hz, 1H), 6.95 (s, 1H), 3.82- 3.76 (m, 2H), 3.45 (s, 2H), 3.43 (s, 2H), 2.07 (s, 2H), 1.87 (s, 1H), 1.76 (dd, J = 9.2, 6.8 Hz, 1H), 1.18 (s, 3H), 1.05 (t, J = 7.2 Hz, 2H), 0.79-0.69 (m, 2H), 0.50-0.41 (m, 2H) |
| 243 | 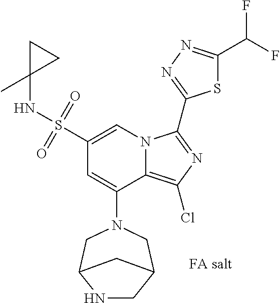 | 5.50 (yield after 2 steps) | Procedure 1 then Procedure 2 | RT 0.362 min; (Method 4); m/z 530.1 (M + H)⁺ (ESI⁺) | ¹H NMR (DMSO-d₆, 400 MHz) 9.66 (s, 1H), 8.34 (s, 1H), 7.69 (t, J = 53.2 Hz, 1H), 6.93 (s, 1H), 3.91 (s, 1H), 3.66 (d, J = 11.2 Hz, 3H), 3.11 (d, J = 10.8 Hz, 2H), 2.89 (d, J = 11.2 Hz, 1H), 2.62- 2.62 (m, 1H), 1.89 (s, 2H), 1.17 (s, 3H), 0.79-0.66 (m, 2H), 0.53-0.41 (m, 2H) |
| 244 | 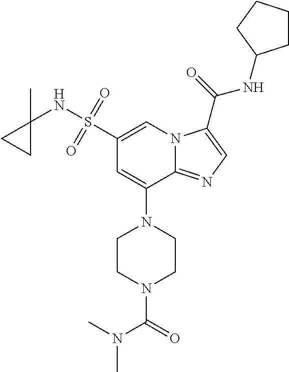 [SMILES: CN(C)C(=O)N1CCN(c2cc(S(=O)(=O)NC3(C)CC3)cn3c(C(=O)NC4CCCC4)cnc23)CC1] | 47.95 | procedure 5 | RT 0.598 min (Method 3); m/z 518.4 (M + H)⁺ (ESI⁺). | ¹H NMR (DMSO-d₆, 400 MHz): 9.69-9.55 (m, 1H), 8.47 (d, J = 7.2 Hz, 1H), 8.38 (s, 1H), 8.25 (br s, 1H), 6.88 (s, 1H), 4.34- 4.25 (m, 1H), 3.57- 3.54 (m, 4H), 3.30 (s, 4H), 2.80 (s, 6H), 1.97-1.92 (m, 2H), 1.73-1.71 (m, 2H), 1.59-1.54 (m, 4H), 1.11 (s, 3H), 0.69- 0.66 (m, 2H), 0.44- 0.41 (m, 2H). |
| 245 | 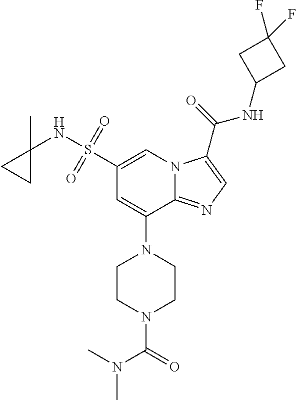 [SMILES: CN(C)C(=O)N1CCN(c2cc(S(=O)(=O)NC3(C)CC3)cn3c(C(=O)NC4CC(F)(F)C4)cnc23)CC1] | 46.67 | procedure 5 | RT 0.574 min (Method 3); m/z 540.1 (M + H)⁺ (ESI⁺). | ¹H NMR (DMSO-d₆, 400 MHz): 9.58 (d, J = 1.4 Hz, 1H), 8.97 (d, J = 6.4 Hz, 1H), 8.35 (s, 1H), 8.27 (s, 1H), 6.89 (s, 1H), 4.33-4.29 (m, 1H), 3.57-3.55 (m, 4H), 3.35-3.34 (m, 4H), 3.09-2.94 (m, 2H), 2.80 (s, 6H), 2.56- 2.53 (m, 2H), 1.10 (s, 3H), 0.71-0.61 (m, 2H), 0.47-0.35 (m, 2H). |
| 246 | 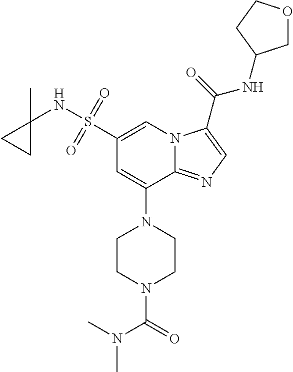 [SMILES: CN(C)C(=O)N1CCN(c2cc(S(=O)(=O)NC3(C)CC3)cn3c(C(=O)NC4CCOC4)cnc23)CC1] | 32.51 | procedure 5 | RT 0.528 min (Method 3); m/z 520.2 (M + H)⁺ (ESI⁺). | ¹H NMR (DMSO-d₆, 400 MHz): 9.61 (d, J = 1.4 Hz, 1H), 8.72 (d, J = 6.8 Hz, 1H), 8.42 (s, 1H), 8.26 (s, 1H), 6.89 (s, 1H), 4.62-4.39 (m, 1H), 3.92-3.80 (m, 2H), 3.78-3.71 (m, 1H), 3.65-3.62 (m, 1H), 3.57-3.55 (m, 4H), 3.36-3.33 (m, 4H), 2.80 (s, 6H), 2.26-2.09 (m, 1H), 2.01-1.87 (m, 1H), 1.10 (s, 3H), 0.73- 0.62 (m, 2H), 0.50- 0.35 (m, 2H). |
| 247 | 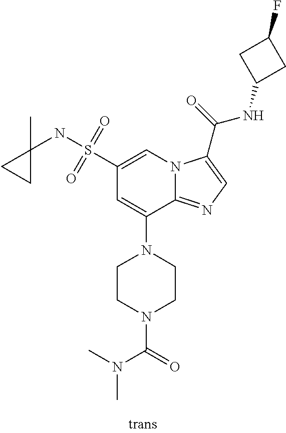 | 50.44 | procedure 5 | RT 0.510 min (Method 3); m/z 522.2 (M + H)⁺ (ESI⁺). | ¹H NMR (DMSO-d₆, 400 MHz):9.58 (d, J = 1.4 Hz, 1H), 8.85 (d, J = 6.8 Hz, 1H), 8.36 (s, 1H), 8.26 (s, 1H), 6.88 (d, J = 1.2 Hz, 1H), 5.41-5.19 (m, 1H), 4.68-4.55 (m, 1H), 3.56-3.45 (m, 4H), 3.43-3.35 (m, 4H), 2.79 (s, 6H), 2.63- 2.52 (m, 4H), 1.10 (s, 3H), 0.70-0.63 (m, 2H), 0.45-0.38 (m, 2H). |
| 248 | 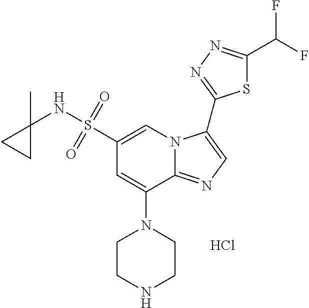 [SMILES: CC1(NS(=O)(=O)c2cc(N3CCNCC3)c3ncc(-c4nnc(C(F)F)s4)n3c2)CC1 ; Cl] | 97.06 | procedure 2 | RT 0.789 min (method 1); m/z 470.0 (M + H)⁺ (ESI⁺). | ¹H NMR (DMSO-d₆, 400 MHz): 9.69 (d, J = 1.2 Hz, 1H), 9.30 (br s, 2H), 8.64 (s, 1H), 8.47 (s, 1H), 7.70 (t, J = 54.8 Hz, 1H), 7.10 (s, 1H), 3.86 (br s, 4H), 3.36 (br s, 4H), 1.14 (s, 3H), 0.75-0.68 (m, 2H), 0.49-0.40 (m, 2H). |
| 249 | 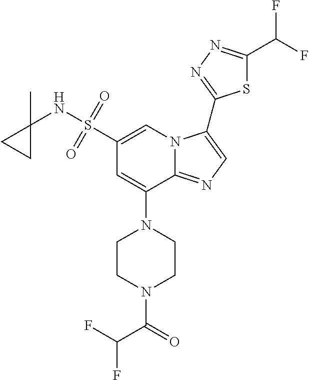 [SMILES: CC1(NS(=O)(=O)c2cc(N3CCN(C(=O)C(F)F)CC3)c3ncc(-c4nnc(C(F)F)s4)n3c2)CC1] | 24.58 | procedure 5 | RT 0.906 min (method 1); m/z 548.0 (M + H)⁺ (ESI⁺). | ¹H NMR (DMSO-d₆, 400 MHz):9.67 (d, J = 1.4 Hz, 1H), 8.64 (s, 1H), 8.41 (s, 1H), 7.70 (t, J = 53.2 Hz, 1H), 7.02 (s, 1H), 6.82 (m, J = 52.4 Hz, 1H), 3.78 (br s, 4H), 3.69-3.68 (m, 4H), 1.13 (s, 3H), 0.74- 0.68 (m, 2H), 0.47- 0.44 (m, 2H). |
| 250 | 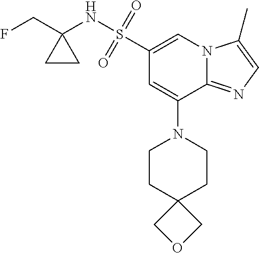 | 46.3 | procedure 6 | RT 0.776 min (method 1); m/z 477.2 (M + H)⁺ (ESI⁺). | ¹H NMR (CDCl₃, 400 MHz): 8.55 (d, J = 4.0 Hz, 1H), 7.56 (s, 1H), 6.64 (d, J = 1.0 Hz, 1H), 6.14 (br s, 1H), 5.51 (s, 1H), 4.52 (s, 4H), 4.42 (d, J = 2.6 Hz, 2H), 4.18 (J = 48.4 Hz, 2H), 4.00 (t, J = 5.4 Hz, 2H), 3.55-3.43 (m, 4H), 2.55-2.54 (m, 2H), 2.17-2.08 (m, 4H), 1.10-1.02 (m, 2H), 0.84-0.77 (m, 2H). |
| 251 | 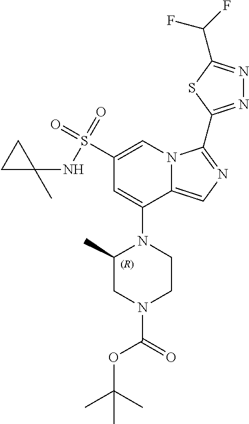 | 35.78 | Procedure 1 | RT 0.526 min (method 1), m/z 584.4 (M + H)⁺ (ESI⁺) | ¹H NMR (CDCl₃, 400 MHz): 9.89 (s, 1H), 7.73 (s, 1H), 7.09 (t, J = 53.6, 1H), 6.66 (s, 1H), 5.06 (s, 1H), 4.10-4.06 (m, 2H), 3.86-3.82 (m, 1H), 3.54-3.44 (m, 2H), 3.33-3.29 (m, 1H), 3.11-3.07 (m, 1H), 1.51 (s, 9H), 1.37 (s, 3H), 1.09 (d, J = 6.4 Hz, 3H), 0.93- 0.91 (m, 2H), 0.60- 0.58 (m, 2H) |
| 252 | 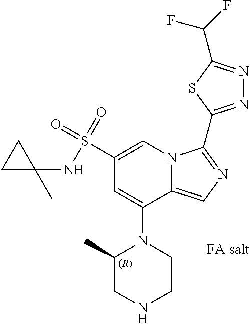 | 92.1 | Procedure 2 | RT 0.359 min (method 1), m/z 484.2 (M + H)⁺ (ESI⁺) | ¹H NMR (CDCl₃, 400 MHz): 9.91 (s, 1H), 8.29 (s, 1H), 7.75 (s, 1H), 7.09 (t, J = 53.6, 1H), 6.81 (s, 1H), 5.48 (br s, 1H), 4.06- 4.00 (m, 1H), 3.55- 3.51 (m, 1H), 3.41- 3.38 (m, 1H), 3.30- 3.27 (m, 1H), 3.22- 3.17 (m, 2H), 3.03- 3.00 (m, 1H), 1.36 (s, 3H), 1.17 (d, J = 6.0 Hz, 3H), 0.94- 0.92 (m, 2H), 0.60- 0.58 (m, 2H) |
| 253 | 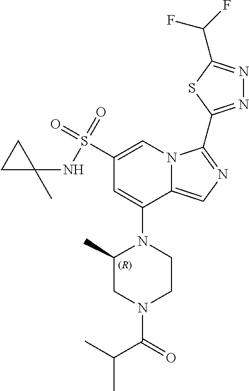 | 5.57 | Procedure 12 | RT 0.483 min (method 1), m/z 554.4 (M + H)⁺ (ESI⁺) | ¹H NMR (CDCl₃, 400 MHz): 9.91 (s, 1H), 7.75 (s, 1H), 7.09 (t, J = 54.0, 1H), 6.67 (s, 1H), 5.08 (s, 1H), 4.52-4.22 (m, 1H), 4.13-4.11 (m, 1H), 4.00-3.96 (m, 0.5H), 3.80-3.75 (m, 1H), 3.65-3.42 (m, 2H), 3.38-3.27 (m, 0.5H), 3.26-3.09 (m, 1H), 3.00-2.72 (m, 1H), 1.38 (s, 3H), 1.26- 1.19 (m, 6H), 1.15- 1.05 (m, 3H), 0.96- 0.90 (m, 2H), 0.61- 0.59 (m, 2H) |
| 255 | 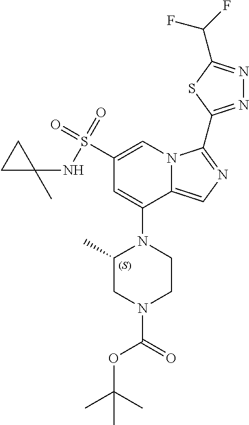 | 35.14 | Procedure 1 | RT 0.532 min (method 1), m/z 584.4 (M + H)⁺ (ESI⁺) | ¹H NMR (CDCl₃, 400 MHz): 9.89 (s, 1H), 7.73 (s, 1H), 7.09 (t, J = 54.0, 1H), 6.66 (s, 1H), 5.07 (s, 1H), 4.14-4.03 (m, 2H), 3.83-3.87 (m, 1H), 3.54-3.44 (m, 2H), 3.37-3.29 (m, 1H), 3.11-3.06 (m, 1H), 1.51 (s, 9H), 1.37 (s, 3H), 1.08 (d, J = 6.4 Hz, 3H), 0.94-0.91 (m, 2H), 0.61-0.59 (m, 2H) |
| 256 | 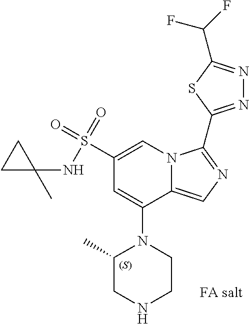 | 95.21 | Procedure 2 | RT 0.365 min (method 1), m/z 484.2 (M + H)⁺ (ESI⁺) | ¹H NMR (CDCl₃, 400 MHz): 9.91 (s, 1H), 8.25 (s, 1H), 7.75 (s, 1H), 7.09 (t, J = 54.0, 1H), 6.81 (s, 1H), 5.46 (s, 1H), 4.06- 4.03 (m, 1H), 3.55- 3.48 (m, 1H), 3.42- 3.38 (m, 1H), 3.30- 3.26 (m, 1H), 3.19- 3.16 (m, 2H), 3.06- 3.00 (m, 1H), 1.36 (s, 3H), 1.17 (d, J = 4.4 Hz, 3H), 0.94-0.91 (m, 2H), 0.61-0.58 (m, 2H) |
| 257 | 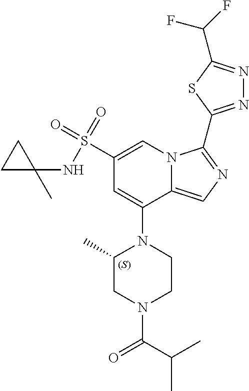 | 16.51 | Procedure 12 | RT 0.478 min (method 1), m/z 554.4 (M + H)⁺ (ESI⁺) | ¹H NMR (CDCl₃, 400 MHz): 9.91 (s, 1H), 7.75 (s, 1H), 7.09 (t, J = 53.6, 1H), 6.67 (s, 1H), 5.09 (s, 1H), 4.48-4.24 (m, 1H), 4.11-4.08 (m, 1H), 3.99-3.92 (m, 0.5H), 3.81-3.72 (m, 1H), 3.63-3.55 (m, 2H), 3.32-3.31 (m, 0.5H), 3.15-3.17 (m, 1H), 2.91-2.83 (m, 1H), 1.38 (s, 3H), 1.25- 1.19 (m, 6H), 1.12- 1.04 (m, 3H), 0.93- 0.91 (m, 2H), 0.61- 0.58 (m, 2H) |
| 258 | 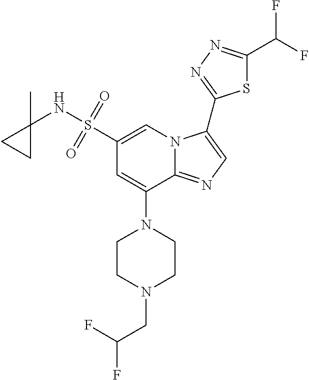 [SMILES: CC1(NS(=O)(=O)c2cc(N3CCN(CC(F)F)CC3)c3ncc(-c4nnc(C(F)F)s4)n3c2)CC1] | 4.8 | Procedure 1 | RT 0.94 min (method 1), m/z 534.1 (M + H)⁺ (ESI⁺) | ¹H NMR (CDCl₃, 400 MHz): 9.91 (s, 1H), 8.81 (s, 1H), 7.11 (t, J = 53.6, 1H), 6.98 (s, 1H), 5.97 (t, J = 56.4, 1H), 5.11 (s, 1H), 3.75-3.65 (m, 4H), 2.91-2.80 (m, 6H), 1.38 (s, 3H), 0.95- 0.85 (m, 2H), 0.62- 0.56 (m, 2H) |
| 259 | 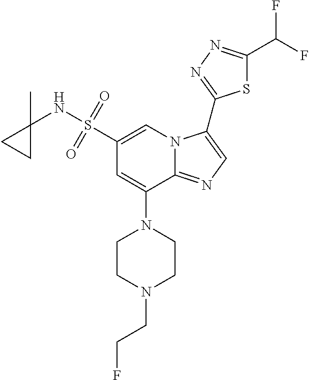 [SMILES: CC1(NS(=O)(=O)c2cc(N3CCN(CCF)CC3)c3ncc(-c4nnc(C(F)F)s4)n3c2)CC1] | 10.9 | Procedure 1 | RT 0.91 min (method 1), m/z 516.1 (M + H)⁺ (ESI⁺) | ¹H NMR (CDCl₃, 400 MHz): 9.91 (s, 1H), 8.81 (s, 1H), 7.19 (t, J = 53.6, 1H), 6.91 (s, 1H), 5.15-5.10 (m, 1H), 4.65-4.50 (m, 2H), 3.70-3.60(m, 4H), 2.90- 2.75 (m, 6H), 1.27 (s, 3H), 0.88-0.80 (m, 2H), 0.52-0.49 (m, 2H) |
| 260 | 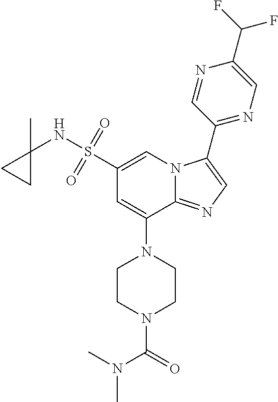 [SMILES: CN(C)C(=O)N1CCN(c2cc(S(=O)(=O)NC3(C)CC3)cn3c(-c4cnc(C(F)F)cn4)cnc23)CC1] | 6.67 (yield after 2 steps) | Procedure 13, followed by Procedure 2 | RT 0.912 min (method 1); m/z 535.2 (M + H)⁺ (ESI⁺) | ¹H NMR (CDCl₃, 400 MHz): 10.10 (s, 1H), 9.10 (s, 1H), 8.99 (s, 1H), 8.35 (s, 1H), 6.91 (s, 1H), 6.76 (t, J = 54.4 Hz, 1H), 5.03 (s, 1H), 3.63 (d, J = 4.8 Hz, 4H), 3.56 (d, J = 4.0 Hz, 4H), 2.90 (s, 6H), 1.35, (s, 3H), 0.89 (t, J = 6.0 Hz, 2H), 0.57 (t, J = 6.0 Hz, 2H). |
| 261 | 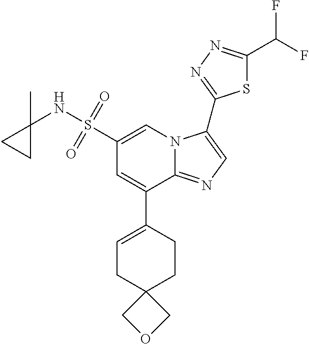 [SMILES: CC1(NS(=O)(=O)c2cc(C3=CCC4(CC3)COC4)c3ncc(-c4nnc(C(F)F)s4)n3c2)CC1] | 21.34 | Procedure 6 | RT 0.866 min (method 1); m/z 508.2 (M + H)⁺ (ESI⁺) | ¹H NMR (CDCl₃, 400 MHz): 10.20 (d, J = 1.6 Hz, 1H), 8.27 (s, 1H), 7.65 (d, J = 1.6 Hz, 1H), 7.10 (t, J = 53.6 Hz, 1H), 7.01 (s, 1H), 5.12 (s, 1H), 4.58 (d, J = 5.6 Hz, 2H), 4.53 (d, J = 5.6 Hz, 2H), 2.72 (d, J = 4.4 Hz, 4H), 2.17, (t, J = 6.4 Hz, 2H), 1.37 (s, 3H), 0.91 (t, J = 5.6 Hz, 2H), 0.61 (t, J = 6.4 Hz, 2H). |
| 262 | 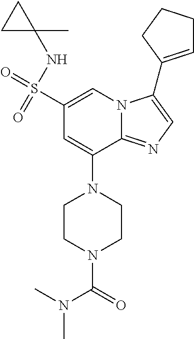 [SMILES: CN(C)C(=O)N1CCN(c2cc(S(=O)(=O)NC3(C)CC3)cn3c(C4=CCCC4)cnc23)CC1] | 49.59 (yield after 2 steps) | Procedure 6, followed by Procedure 2 | RT 0.866 min (method 1); m/z 473.4 (M + H)⁺ (ESI⁺) | ¹H NMR (CDCl₃, 400 MHz): 8.70 (d, J = 1.6 Hz, 1H), 7.57 (s, 1H), 6.69 (s, 1H), 6.22 (s, 1H), 5.05 (s, 1H), 3.58 (d, J = 5.2 Hz, 4H), 3.53 (d, J = 5.6 Hz, 4H), 2.89 (s, 6H), 2.87-2.83 (m, 2H), 2.70-2.67 (m, 2H), 2.10-2.02 (m, 2H), 1.31, (s, 3H), 0.84 (t, J = 6.0 Hz, 2H), 0.55- 0.52 (m, 2H). |
| 263 | 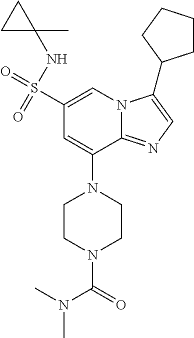 [SMILES: CN(C)C(=O)N1CCN(c2cc(S(=O)(=O)NC3(C)CC3)cn3c(C4CCCC4)cnc23)CC1] | 82.9 | Procedure 7 | RT 0.776 min (method 1); m/z 475.3 (M + H)⁺ (ESI⁺) | ¹H NMR (CDCl₃, 400 MHz): 8.29 (s, 1H), 7.41 (s, 1H), 6.61 (s, 1H), 4.97 (s, 1H), 3.60 (d, J = 4.4 Hz, 4H), 3.52 (d, J = 4.4 Hz, 4H), 3.33-3.25 (m, 1H), 2.89 (s, 6H), 2.24-2.22 (m, 2H), 1.84-1.71 (m, 6H), 1.71, (s, 3H), 0.83 (t, J = 6.0 Hz, 2H), 0.52 (t, J = 6.0 Hz, 2H). |
| 264 | 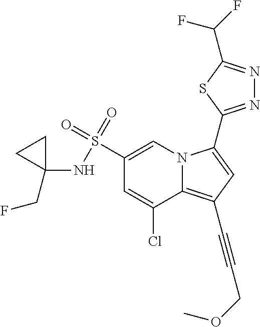 [SMILES: COCC#Cc1cc(-c2nnc(C(F)F)s2)n2cc(S(=O)(=O)NC3(CF)CC3)cc(Cl)c12] | 21.87 | Procedure 10 | RT 0.532 min (Method 4); m/z 526.8 (M + Na)⁺ (ESI⁺) | ¹H NMR (CDCl₃, 400 MHz): 10.35 (s, 1H), 7.57 (s, 1H), 7.42 (s, 1H), 7.22-6.95 (m, 1H), 5.79-5.21 (m, 1H), 4.40 (s, 2H), 4.34 (d, J = 48.4 Hz, 2H), 3.51 (s, 3H), 1.12-1.16 (m, J = 2.8 Hz, 2H), 0.92-0.88 (m, 2H). |
| 265 | 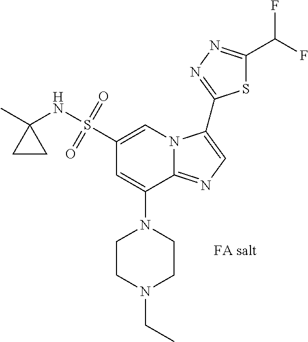 | 32.80 | Procedure 1 | RT 0.512 min (Method 3); m/z 498.2 (M + H)⁺ (ESI⁺) | ¹H NMR (CDCl₃, 400 MHz): 9.91 (d, J = 1.6 Hz, 1H), 8.43 (s, 1H), 8.18 (s, 1H), 6.97-7.26 (m, 2H), 5.48 (br, 1H), 3.79 (s, 4H), 2.98-2.96 (m, 4H), 2.75 (m, 2H), 1.35 (s, 3H), 1.26 (t, J = 7.27 Hz, 3H), 0.88-0.95 (m, 2H), 0.55-0.61 (m, 2H). |
| 266 | 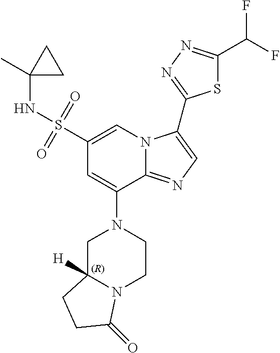 | 4.36 | Procedure 1 | RT 0.590 min (Method 3); m/z 524.2 (M + H)⁺ (ESI⁺) | ¹H NMR (DMSO-d₆, 400 MHz): 9.67 (s, 1H), 8.64 (s, 1H), 8.43-8.39 (m, 1H), 7.70 (t, J = 53.2 Hz, 1H), 7.06 (s, 1H), 4.53-4.49 (m, 1H), 4.45-4.41 (m, 1H), 4.03-3.97 (m, 1H), 3.87-3.76 (m, 1H), 3.42-3.36 (m, 1H), 3.12-3.00 (m, 1H), 2.85-2.80 (m, 1H), 2.73-2.68 (m, 1H), 2.33-2.27 (m, 1H), 2.27-2.18 (m, 1H), 1.75-1.56 (m, 1H), 1.13 (s, 3H), 0.77- 0.63 (m, 2H), 0.49- 0.39 (m, 2H) |
| 267 | 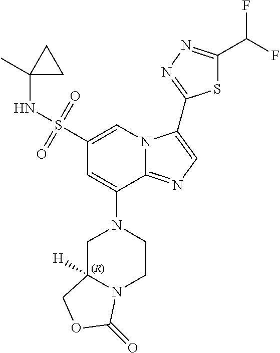 | 10.84 | Procedure 1 | RT 0.600 min (Method 3); m/z 526.1 (M + H)⁺ (ESI⁺) | ¹H NMR (DMSO-d₆, 400 MHz): 9.67 (s, 1H), 8.63 (s, 1H), 8.43-8.39 (m, 1H), 7.70 (t, J = 53.2 Hz, 1H), 7.06 (s, 1H), 4.54-4.42 (m, 2H), 4.38-4.34 (m, 1H), 4.18-3.99 (m, 2H), 3.84-3.67 (m, 1H), 3.30-3.26 (m, 1H), 2.98-2.84 (m, 2H), 1.13 (s, 3H), 0.74- 0.67 (m, 2H), 0.48- 0.40 (m, 2H) |
| 268 | 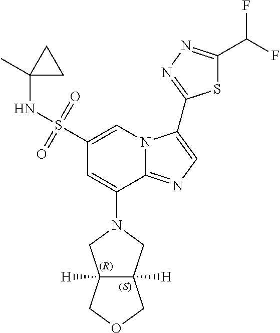 | 18.1 | Procedure 1 | RT 0.647 min (Method 3); m/z 497.1 (M + H)⁺ (ESI⁺) | ¹H NMR (DMSO-d₆, 400 MHz): 9.50 (s, 1H), 8.56 (s, 1H), 8.39-8.31 (s, 1H), 7.69 (t, J = 53.2 Hz, 1H), 6.64 (s, 1H), 3.90-3.84 (m, 4H), 3.78-3.72 (m, 2H), 3.66-3.61 (m, 2H), 3.10-3.04 (m, 2H), 1.15 (s, 3H), 0.74- 0.65 (m, 2H), 0.48- 0.38 (m, 2H) |
| 269 | 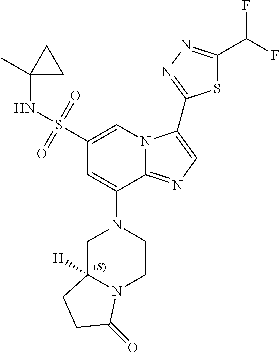 | 7.56 | Procedure 1 | RT 0.592 min (Method 3); m/z 524.2 (M + H)⁺ (ESI⁺) | ¹H NMR (DMSO-d₆, 400 MHz): 9.67 (s, 1H), 8.63 (s, 1H), 8.49-8.37 (m, 1H), 7.70 (t, J = 53.2 Hz, 1H), 7.06 (s, 1H), 4.51-4.47 (m, 1H), 4.45-4.39 (m, 1H), 4.04-3.94 (m, 1H), 3.84-3.79 (m, 1H), 3.38-3.30 (m, 1H), 3.06-3.02 (m, 1H), 2.85-2.79 (m, 1H), 2.70-2.65 (m, 1H), 2.33-2.29 (m, 1H), 2.25-2.19 (m, 1H), 1.69-1.64 (m, 1H), 1.13 (s, 3H), 0.74- 0.67 (m, 2H), 0.49- 0.41 (m, 2H) |
| 270 | 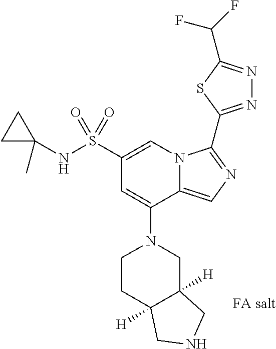 [SMILES: CC1(NS(=O)(=O)c2cc(N3CC[C@@H]4CNC[C@@H]4C3)c3cnc(-c4nnc(C(F)F)s4)n3c2)CC1] | 40.41 | Procedure 2 | RT 0.326 min (method 5); m/z 510.2 (M + H)⁺ (ESI⁺) | ¹H NMR (D₂O, 400 MHz): 9.18 (s, 1H), 8.42 (s, 1H), 7.49 (s, 1H), 7.29 (t, J = 53.2 Hz, 1H), 6.51 (s, 1H), 3.52-3.31 (m, 7H), 3.20 3.05 (m, 1H), 2.85-2.75 (m, 1H), 2.73-2.61 (m, 1H), 2.10-2.00 (m, 1H), 1.85-1.70 (m, 1H), 1.16 (s, 3H), 0.78-0.66 (m, 2H), 0.50-0.37 (m, 2H). |
| 271 | 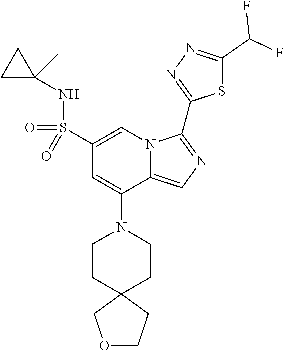 [SMILES: CC1(NS(=O)(=O)c2cc(N3CCC4(CCOC4)CC3)c3cnc(-c4nnc(C(F)F)s4)n3c2)CC1] | 10.56 | Procedure 1 | RT 0.464 min (method 5); m/z 525.2 (M + H)⁺ (ESI⁺) | ¹H NMR (CDCl₃, 400 MHz): 9.84 (s, 1H), 7.71 (s, 1H), 7.08 (t, J = 53.6 Hz, 1H), 6.65 (s, 1H), 5.05 (s, 1H), 3.94 (t, J = 7.2 Hz, 2H), 3.67 (s, 2H), 3.33 ( t, J = 5.2 Hz, 4H), 1.91-1.83 (m, 6H), 1.39 (s, 3H), 0.99 0.91 (m, 2H), 0.65 0.57 (m, 2H). |
| 272 | 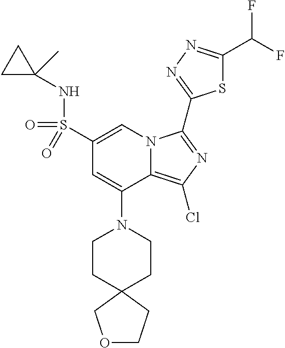 [SMILES: CC1(NS(=O)(=O)c2cc(N3CCC4(CCOC4)CC3)c3c(Cl)nc(-c4nnc(C(F)F)s4)n3c2)CC1] | 2.83 | Procedure 1 | RT 0.510 min (method 5); m/z 559.2 (M + H)⁺ (ESI⁺) | ¹H NMR (DMSO-d₆, 400 MHz): 9.60 (s, 1H), 8.48 (s, 1H), 7.68 (t, J = 53.6 Hz, 1H), 6.83 (s, 1H), 3.79 (t, J = 7.6 Hz, 2H), 3.55 (s, 2H), 3.14-3.02 (m, 4H), 1.85-1.73 (m, 6H), 1.17 (s, 3H), 0.76- 0.69 (m, 2H), 0.50- 0.40 (m, 2H). |
| 273 | 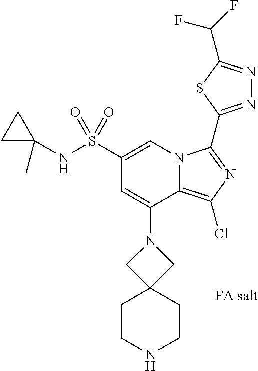 [SMILES: CC1(NS(=O)(=O)c2cc(N3CC4(CCNCC4)C3)c3c(Cl)nc(-c4nnc(C(F)F)s4)n3c2)CC1] | 4.44 (yield after 2 steps) | Procedure 1 then Procedure 2 | RT 0.394 min (method 5); m/z 544.2 (M + H)⁺ (ESI⁺) | ¹H NMR ((DMSO-d₆, 400 MHz) 9.48 (s, 1H), 8.37-8.54 (m, 1H), 8.31-8.37 (m, 1H), 7.68 (t, J = 53.6 Hz, 1H), 6.32 (s, 1H), 3.98 (s, 4H), 2.76- 2.89 (m, 4H), 1.74- 1.82 (m, 4H), 1.18 (s, 3H), 0.72-0.76 (m, 2H), 0.44-0.48 (m, 2H) |
| 274 | 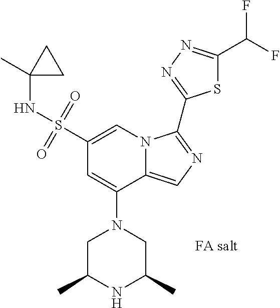 [SMILES: C[C@@H]1CN(c2cc(S(=O)(=O)NC3(C)CC3)cn3c(-c4nnc(C(F)F)s4)ncc23)C[C@H](C)N1 ; "FA salt"] | 4.08 (yield after 2 steps) | Procedure 1 then Procedure 2 | RT 0.361 min (method 5); m/z 498 (M + H)⁺ (ESI⁺) | ¹H NMR (DMSO-d₆, 400 MHz) 9.57 (s, 1H), 8.44 (s, 1H), 8.22 (s, 1H), 8.01 (s, 1H), 7.68 (t, J = 53.6 Hz, 1H), 6.65 (s, 1H), 3.58-3.61 (m, 2H), 3.04-3.08 (m, 2H), 2.39-2.42 (m, 2H), 1.14 (s, 3H), 1.07 (d, J = 6.4 Hz, 6H), 0.70- 0.74 (m, 2H), 0.42- 0.47 (m, 2H) |
| 275 | 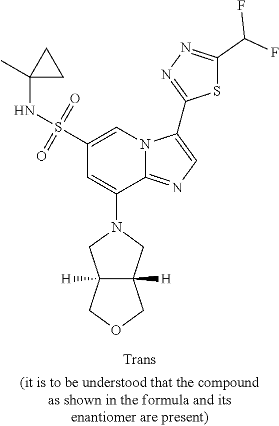 [SMILES: CC1(NS(=O)(=O)c2cc(N3C[C@@H]4COC[C@H]4C3)c3ncc(-c4nnc(C(F)F)s4)n3c2)CC1] | 25.90 | Procedure 1 | RT 0.623 min (method 3); m/z 497.1 (M + H)⁺ (ESI⁺) | ¹H NMR (CDCl₃, 400 MHz): 9.61 (d, J = 1.2 Hz, 1H), 8.09 (s, 1H), 7.09 (t, J = 53.6 Hz, 1H), 6.51 (d, J = 1.2 Hz, 1H), 5.09 (s, 1H), 4.17-4.07 (m, 4H), 3.69-3.59 (m, 4H), 2.68 2.61 (m, 2H), 1.37 (s, 3H), 0.95-0.92 (m, 2H), 0.60-0.56 (m, 2H) |
| 276 | 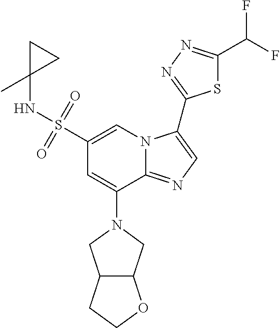 [SMILES: CC1(NS(=O)(=O)c2cc(N3CC4CCOC4C3)c3ncc(-c4nnc(C(F)F)s4)n3c2)CC1] | 35.50 | Procedure 1 | RT 0.638 min (method 3); m/z 497.0 (M + H)⁺ (ESI⁺) | ¹H NMR (CDCl₃, 400 MHz): 9.69 (s, 1H), 8.11 (s, 1H), 7.09 (t, J = 53.6 Hz, 1H), 6.59 (s, 1H), 5.08 (s, 1H), 4.70 (t, J = 5.6 Hz, 1H), 4.23 (d, J = 12.0 Hz, 1H), 4.07-3.98 (m, 1H), 3.95-3.91 (m, 1H), 3.90-3.88 (m, 2H), 3.81 (dd, J = 12.0, 5.6 Hz, 1H), 3.11-3.07 (m, 1H), 2.30-2.21 (m, 1H), 2.02-1.96 (m, 1H), 1.36 (s, 3H), 0.94 0.92 (m, 2H), 0.58-0.56 (m, 2H) |
| 277 | 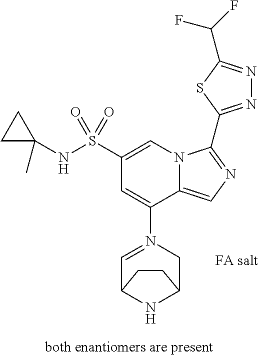 | 7.53 (yield after 2 steps) | Procedure 1 then Procedure 2 | RT 0.352 min; (method 5); m/z 493.1 (M + H)⁺ (ESI⁺) | ¹H NMR (DMSO-d₆, 400 MHz) 9.84 (s, 1H), 8.51 (br, 1H), 8.31 (s, 1H), 8.12 (s, 1H), 7.69 (t, J = 53.2 Hz, 1H), 7.24 (s, 1H), 6.80 (d, J = 5.6 Hz, 1H), 3.85- 3.94 (m, 2H), 2.92- 2.97 (dd, J = 16.8 Hz, 5.6 Hz, 1H), 2.27 (d, J = 16.8 Hz, 1H), 2.00-2.12 (m, 2 H), 1.86-1.90 (m, 1H), 1.64-1.79 (m, 1H), 1.16 (s, 3H), 0.65-0.80 (m, 2H), 0.41-0.53 (m, 2H). |

### Preparation of Example 278

### 4-(1-chloro-3(5-(difluoromethyl)-1,3,4-thiadiazol-2-yl)-6-(N-(1-methylcyclopropyl)sulfamoyl)imidazo[1,5-a]pyridin-8-yl)-N-(2-dimethylamino)ethyl)-N-methylpiperazine-1-carboxamide

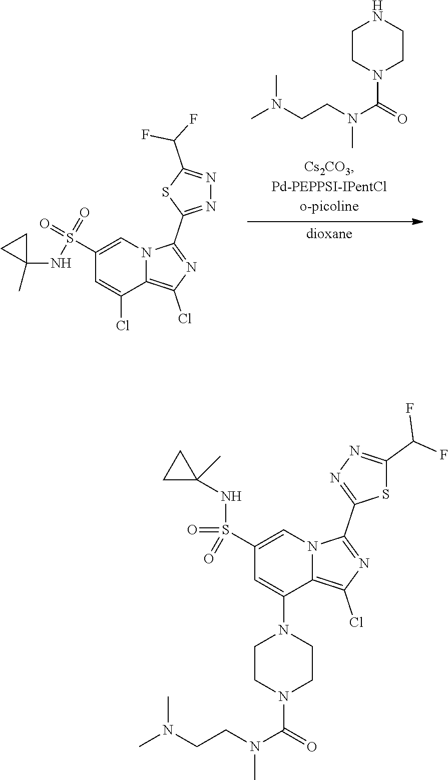 [SMILES: CC1(NS(=O)(=O)c2cc(Cl)c3c(Cl)nc(-c4nnc(C(F)F)s4)n3c2)CC1 ; CN(C)CCN(C)C(=O)N1CCNCC1 ; CN(C)CCN(C)C(=O)N1CCN(c2cc(S(=O)(=O)NC3(C)CC3)cn3c(-c4nnc(C(F)F)s4)nc(Cl)c23)CC1]

To a solution of 1,8-dichloro-3-(5-(difluoromethyl)-1,3,4-thiadiazol-2-yl)-N-(1-methylcyclopropyl)imidazo[1,5-a]pyridine-6-sulfonamide (30 mg, 0.0660 mmol) in dioxane (1 mL) was added N-(2-(dimethylamino)ethyl)-N-methylpiperazine-1-carboxamide hydrochloride (33 mg, 0.132 mmol, HCl salt), Cs₂CO₃ (54 mg, 0.165 mmol) and Pd-PEPPSI-IPentCl o-picoline (6.4 mg, 0.0066 mmol). The mixture was degassed and purged with N₂ (3×)before it was stirred at 100° C. for 2 h under a N₂ atmosphere. The reaction mixture was filtered and the filtrate was concentrated under vacuum. The residue was purified by preparative HPLC (column: Phenomenex luna C18 150*25 mm*10 μm; mobile phase: A: 0.225% formic acid in water, B: MeCN; B %: 20%-50%, 10 min) and lyophilized to give the product 4-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-N-(2-(dimethylamino)ethyl)-N-methylpiperazine-1-carboxamide (5.4 mg, 0.00821 mmol, 12.4% yield) as a yellow solid.

RT 0.415 min (method 4); m/z 632.3 (M+H)⁺ (ESI⁺), ¹H NMR (CDCl₃, 400 MHz): 9.89 (s, 1H), 7.08 (t, J=53.6 Hz, 1H), 6.76 (s, 1H), 5.34 (br, 1H), 3.61-3.51 (m, 4H), 3.48 (t, J=6.8 Hz, 2H), 3.25-3.15 (m, 4H), 2.98 (s, 3H), 2.76 (t, J=6.8 Hz, 2H), 2.47 (s, 6H), 1.38 (s, 3H), 0.94-0.90 (m, 2H), 0.63-0.58 (m, 2H).

### Preparation of Example 279a

### tert-butyl-3-(3-(5-(difluoromethyl)-1,3,4-thiadiazol-2-yl)-6-(N-(1-methylcyclopropyl)sulfamoyl)imidazo[1,5-a]pyridin-8-yl)-8-azabicyclo[3.2.1]oct-2-ene-8-carboxylate

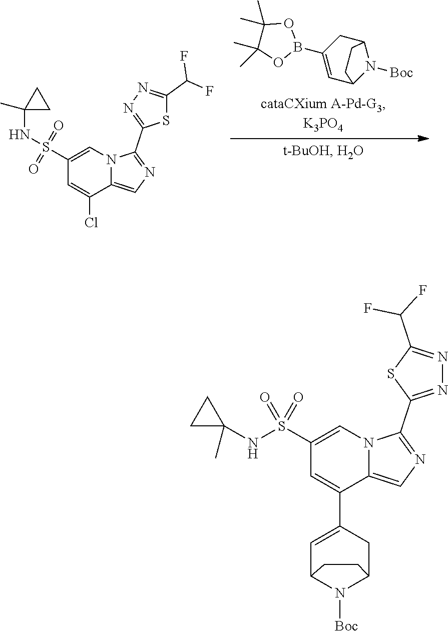 [SMILES: CC1(NS(=O)(=O)c2cc(Cl)c3cnc(-c4nnc(C(F)F)s4)n3c2)CC1 ; CC(C)(C)OC(=O)N1C2C=C(B3OC(C)(C)C(C)(C)O3)CC1CC2 ; CC1(NS(=O)(=O)c2cc(C3=CC4CCC(C3)N4C(=O)OC(C)(C)C)c3cnc(-c4nnc(C(F)F)s4)n3c2)CC1]

To a solution of 8-chloro-3-(5-(difluoromethyl)-1,3,4-thiadiazol-2-yl)-N-(1-methylcyclopropyl)imidazo[1,5-a]pyridine-6-sulfonamide (100 mg, 0.238 mmol) in tert-butanol (2 mL) were added water (0.2 mL), K₃P04 (51 mg, 0.238 mmol), cataCXium A-Pd-G3 (17 mg, 0.0238 mmol) and tert-butyl 3-(4,4,5,5-tetramethyl-1,3,2-dioxaborolan-2-yl)-8-azabicyclo[3.2.1]oct-2-ene-8-carboxylate (240 mg, 0.715 mmol). The mixture was stirred at 60° C. for 16 h under a N₂ atmosphere. The resulting mixture was concentrated and the residue was purified by preparative TLC (petroleum ether:ethyl acetate=2:1) to give the product tert-butyl-3-(3-(5-(difluoromethyl)-1,3,4-thiadiazol-2-yl)-6-(N-(1-methylcyclopropyl)sulfamoyl)imidazo[1,5-a]pyridin-8-yl)-8-azabicyclo[3.2.1]oct-2-ene-8-carboxylate (35 mg, 0.0555 mmol, 23.3% yield) as a yellow solid.

RT 0.550 min (method 4); m/z 593.1 (M+H)⁺ (ESI⁺), ¹H NMR (DMSO-d₆, 400 MHz): 9.84 (s, 1H), 8.50 (s, 1H), 8.03 (s, 1H), 7.69 (t, J=53.2 Hz, 1H), 7.26 (s, 1H), 6.86 (d, J=5.2 Hz, 1H), 4.46-4.52 (m, 1H), 4.35-4.43 (m, 1H), 3.03-3.18 (m, 1H), 2.28-2.35 (m, 1H), 2.01-2.12 (m, 2H), 1.75-1.88 (m, 2H), 1.41 (s, 9H), 1.15 (s, 3H), 0.67-0.74 (m, 2H), 0.48-0.44 (m, 2H).

### Preparation of Example 279b

### tert-butyl-3-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-8-azabicyclo[3.2.1]oct-2-ene-8-carboxylate

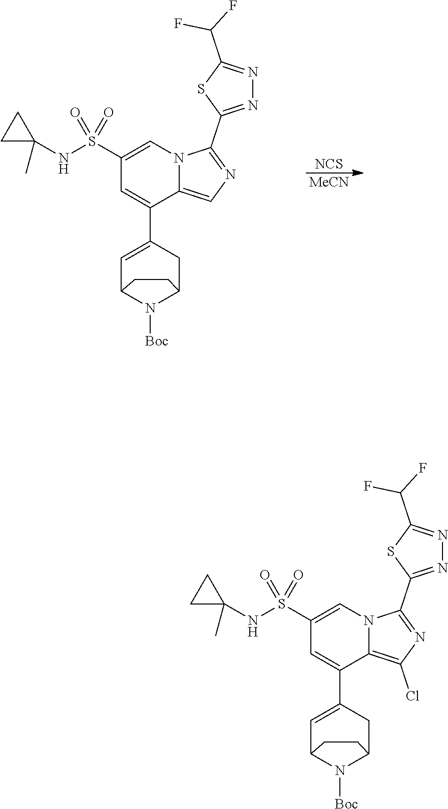 [SMILES: "MeCN" ; CC1(NS(=O)(=O)c2cc(C3=CC4CCC(C3)N4C(=O)OC(C)(C)C)c3cnc(-c4nnc(C(F)F)s4)n3c2)CC1 ; "NCS" ; CC1(NS(=O)(=O)c2cc(C3=CC4CCC(C3)N4C(=O)OC(C)(C)C)c3c(Cl)nc(-c4nnc(C(F)F)s4)n3c2)CC1]

To a solution of tert-butyl-3-(3-(5-(difluoromethyl)-1,3,4-thiadiazol-2-yl)-6-(N-(1-methylcyclopropyl)sulfamoyl)imidazo[1,5-a]pyridin-8-yl)-8-azabicyclo[3.2.1]oct-2-ene-8-carboxylate (35 mg, 0.0591 mmol) in MeCN (1.2 mL) was added NCS (13 mg, 0.094 mmol) at 25° C. The mixture was stirred at 25° C. for 18 h. NCS (6.5 mg, 0.0472 mmol) was added to the solution and the mixture was stirred for 16 h. The resulting solution was diluted with water (10 mL) and extracted with EtOAc (15 mL, 3×). The combined organic phases were washed with brine (15 mL, 2×), dried with anhydrous Na₂SO₄, filtered and the filtrate was concentrated under vacuum to give the product tert-butyl-3-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-8-azabicyclo[3.2.1]oct-2-ene-8-carboxylate (32 mg, 0.0459 mmol, 77.77% yield) as a yellow solid.

RT 0.577 min (method 4); m/z 571.0 (M−56+H)⁺ (ESI⁺), ¹H NMR (DMSO-d₆, 400 MHz): 9.83 (s, 1H), 8.56 (s, 1H), 7.70 (t, J=53.2 Hz, 1H), 7.10 (s, 1H), 6.25 (d, J=5.2 Hz, 1H), 4.45 (t, J=5.2 Hz, 1H), 4.37-4.28 (m, 1H), 2.26-2.16 (m, 2H), 2.08-2.00 (m, 2H), 1.96-2.03 (m, 2H), 1.43 (s, 9H), 1.23 (s, 3H), 0.72 (m, 2H), 0.47 (m, 2H).

### Preparation of Example 279

### 8-azabicyclo[3.2.1]oct-2-en-3-yl)-1-chloro-3-(5-(difluoromethyl)-1,3,4-thiadiazol-2-yl)-N-(1-methylcyclopropyl)imidazo[1,5-a]pyridine-6-sulfonamide formate

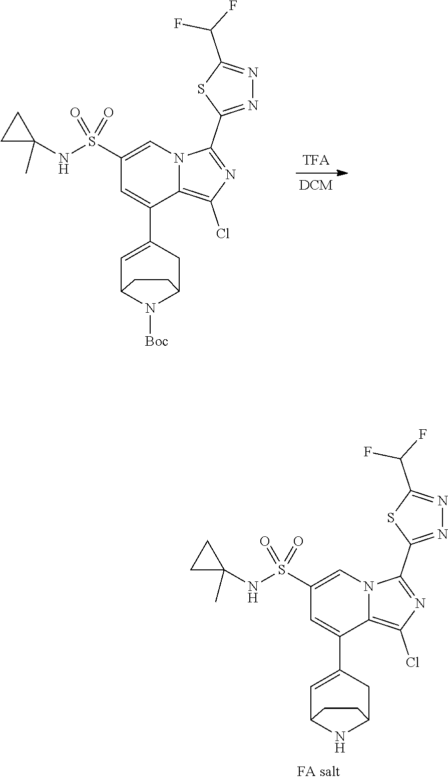 [SMILES: CC1(NS(=O)(=O)c2cc(C3=CC4CCC(C3)N4C(=O)OC(C)(C)C)c3c(Cl)nc(-c4nnc(C(F)F)s4)n3c2)CC1 ; CC1(NS(=O)(=O)c2cc(C3=CC4CCC(C3)N4)c3c(Cl)nc(-c4nnc(C(F)F)s4)n3c2)CC1]

A solution of tert-butyl-3-(1-chloro-3-(5-(difluoromethyl)-1,3,4-thiadiazol-2-yl)-6-(N-(1-methylcyclopropyl)sulfamoyl)imidazo[1,5-a]pyridin-8-yl)-8-azabicyclo[3.2.1]oct-2-ene-8-carboxylate (25 mg, 0.0399 mmol) in TFA (1.0 mL) and DCM (0.5 mL) was stirred at 25° C. for 1 h. The mixture was concentrated and the residue was purified by reversed-phase HPLC (column: Phenomenex luna C18 150*25 mm*10 μm; mobile phase: A: 0.225% formic acid in water, B: MeCN; B %: 14%-44%, 10 min) to give the product 8-azabicyclo[3.2.1]oct-2-en-3-yl)-1-chloro-3-(5-(difluoromethyl)-1,3,4-thiadiazol-2-yl)-N-(1-methylcyclopropyl)imidazo[1,5-a]pyridine-6-sulfonamide formate (5.0 mg, 0.00867 mmol, 21.76% yield, FA salt) as a yellow solid.

RT 0.393 min (method 4); m/z 527.1 (M+H)⁺ (ESI⁺), ¹H NMR (DMSO-d₆, 400 MHz): 9.84 (s, 1H), 8.57 (br, 1H), 8.31 (s, 1H), 7.70 (t, J=53.2 Hz, 1H), 7.15 (s, 1H), 6.17 (d, J=5.2 Hz, 1H), 3.89-3.82 (m, 2H), 2.89-2.78 (m, 1H), 2.14-2.24 (m, 1H), 2.11-1.99 (m, 2H), 1.92-1.81 (m, 2H), 1.19 (s, 3H), 0.74-0.72 (m, 2H), 0.52-0.50 (m, 2H).

### Preparation of Intermediate 280.1

### benzyl 4-(2-chloropropanoyl)piperazine-1-carboxylate

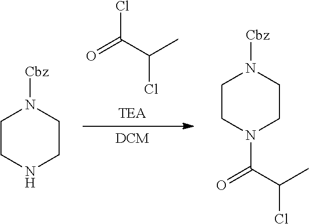

To a solution of benzyl piperazine-1-carboxylate (2.00 g, 9.08 mmol) in DCM (20 mL) was added TEA (2.5 mL, 18.2 mmol) and 2-chloropropanoyl chloride (1.27 g, 9.99 mmol) at 0° C. before the mixture was stirred at 25° C. for 1 h. The resulting mixture was concentrated under vacuum to give the crude product benzyl 4-(2-chloropropanoyl)piperazine-1-carboxylate (2.10 g, crude, 80.8% purity based on LMCS) as a white solid.

RT 0.385 min (method 4); m/z 311.2 (M+H)⁺ (ESI⁺), ¹H NMR (CDCl₃, 400 MHz): 7.40-7.27 (m, 5H), 5.12 (s, 2H) 3.80-3.50 (m, 4H), 3.49-3.32 (m, 4H), 3.39 (q, J=6.8 Hz, 1H), 1.65 (d, J=6.8 Hz, 3H).

### Preparation of Intermediate 280.2

### benzyl 4-(dimethylalanyl)piperazine-1-carboxylate formate

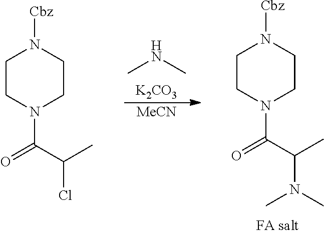

To a solution of benzyl 4-(2-chloropropanoyl)piperazine-1-carboxylate (500 mg, 1.30 mmol, 80.8% purity) in MeCN (5 mL) was added dimethylamine hydrochloride (159 mg, 1.95 mmol, HCl salt). The mixture was stirred at 80° C. for 16 h. The resulting mixture was filtered, and the filtrate was concentrated under vacuum to give a residue, which was purified by preparative HPLC (column: Unisil 3-100 C18 Ultra 150*25 mm*10 μm; mobile phase: A: 0.225% formic acid in water, B: MeCN; B %: 0%-30%, 10 min) and lyophilized directly to give the product benzyl 4-(dimethylalanyl)piperazine-1-carboxylate (70 mg, 0.184 mmol, 14.1% yield, 96.0% purity, FA salt) as a colorless oil.

RT 0.253 min (method 4); m/z 320.2 (M+H)⁺ (ESI⁺), ¹H NMR (DMSO-d₆, 400 MHz): 8.30 (br, 1H), 7.57-7.16 (m, 5H), 5.10 (s, 2H), 3.72-3.51 (m, 9H), 2.14 (s, 6H), 1.00 (d, J=6.6 Hz, 3H).

### Preparation of Intermediate 280.3

### 2-(dimethylamino)-1-(piperazin-1-yl)propan-1-one

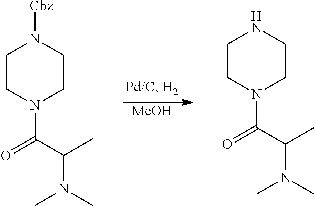 [SMILES: "MeOH" ; CC(C(=O)N1CCN(C(=O)OCc2ccccc2)CC1)N(C)C ; CC(C(=O)N1CCNCC1)N(C)C]

To a suspension of Pd/C (20 mg, 10% purity) in MeOH (2 mL) was added benzyl 4-(dimethylalanyl)piperazine-1-carboxylate formate (50 mg, 0.131 mmol, 96.0% purity, FA salt). The suspension was degassed under vacuum and purged with H₂ (3×) before the mixture was stirred at 25° C. for 4 h under a H₂ atmosphere. The suspension was filtered through a pad of celite, and the cake was washed by MeOH (3 mL, 4×). The combined filtrates were concentrated in vacuum to give the crude product 2-(dimethylamino)-1-(piperazin-1-yl)propan-1-one (34 mg, 0.110 mmol, 83.80% yield) as a colorless oil which was used in the next step without further purification.

RT 0.060 min (method 4); m/z 186.2 (M+H)⁺ (ESI⁺).

### Preparation of Example 280

### 3-(5-(difluoromethyl)-1,3,4-thiadiazol-2-yl)-8-(4-(dimethylalanyl)piperazin-1-yl)-N-(1-methylcyclopropyl)imidazo[1,5-a]pyridine-6-sulfonamide formate

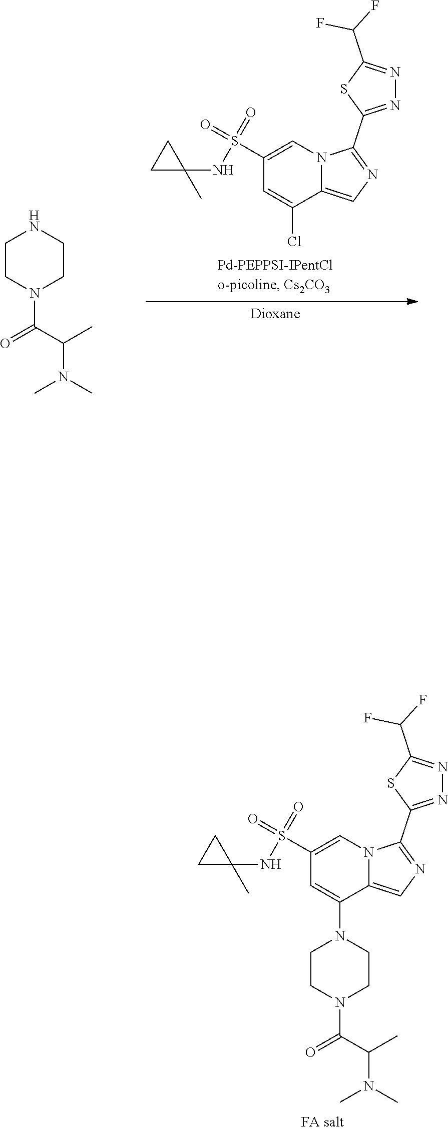 [SMILES: CC(C(=O)N1CCNCC1)N(C)C ; CC1(NS(=O)(=O)c2cc(Cl)c3cnc(-c4nnc(C(F)F)s4)n3c2)CC1 ; CC(C(=O)N1CCN(c2cc(S(=O)(=O)NC3(C)CC3)cn3c(-c4nnc(C(F)F)s4)ncc23)CC1)N(C)C]

To a solution of 8-chloro-3-(5-(difluoromethyl)-1,3,4-thiadiazol-2-yl)-N-(1-methylcyclopropyl)imidazo[1,5-a]pyridine-6-sulfonamide (30 mg, 0.0715 mmol) in dioxane (1 mL) was added 2-(dimethylamino)-1-(piperazin-1-yl)propan-1-one (33 mg, 0.179 mmol), Cs₂CO₃ (70 mg, 0.21 mmol) and Pd-PEPPSI-IPentCl o-picoline (7.0 mg, 0.00715 mmol). The mixture was degassed and purged with N₂ (3×) before it was stirred at 100° C. for 16 h under a N₂ atmosphere. The reaction mixture was cooled to 25° C. and filtered. The filtrate was concentrated under vacuum to give a residue, which was purified by preparative HPLC (column: Phenomenex C18 150*25 mm*10 μm; mobile phase: A: 0.225% formic acid in water, B: MeCN; B %: 14%-44%, 10 min) and lyophilized directly to give the product 3-(5-(difluoromethyl)-1,3,4-thiadiazol-2-yl)-8-(4-(dimethylalanyl)piperazin-1-yl)-N-(1-methylcyclopropyl)imidazo[1,5-a]pyridine-6-sulfonamide formate (3.76 mg, 0.00609 mmol, 8.53% yield, 99.63% purity, FA salt) as a yellow solid.

RT 0.337 min (method 4); m/z 569.3 (M+H)⁺ (ESI⁺), ¹H NMR (CDCl₃, 400 MHz): 9.88 (s, 1H), 8.37 (s, 1H), 7.72 (s, 1H), 7.08 (t, J=53.6 Hz, 1H), 6.69 (s, 1H), 5.56 (br, 1H), 4.16-4.01 (m, 2H), 3.98 (q, J=6.8 Hz, 1H), 3.85-3.70 (m, 2H), 3.45-3.32 (m, 3H), 3.28-3.17 (m, 1H), 2.52 (s, 6H), 1.38 (s, 3H), 1.34 (d, J=6.8 Hz, 3H), 0.96-0.88 (m, 2H), 0.63-0.55 (m, 2H).

### Preparation of Intermediate 281.1

### benzyl 4-(1-methylazetidine-3-carbonyl)piperazine-1-carboxylate formate

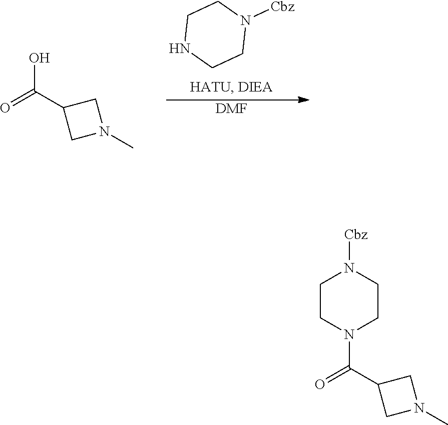

To a solution of 1-methylazetidine-3-carboxylic acid (300 mg, 2.61 mmol) in DMF (15 mL) was added HATU (604 mg, 1.59 mmol) and DIEA (0.33 mL, 1.99 mmol). The mixture was stirred at 25° C. for 30 min, then benzyl piperazine-1-carboxylate (175 mg, 0.794 mmol) was added, and the mixture was stirred at 25° C. for 16 h. The resulting mixture was poured into water (30 mL), extracted with ethyl acetate (30 mL; 2×) and washed with brine (30 mL; 3×). The combined organic phases were dried over anhydrous Na₂SO₄, filtered and the filtrate was concentrated under vacuum to give a residue, which was purified by preparative HPLC (column: Unisil 3-100 C18 Ultra 150*25 mm*10 μm; mobile phase: A: 0.225% formic acid in water, B: MeCN; B %: 0%-28%, 10 min) and lyophilized directly to give the product benzyl 4-(1-methylazetidine-3-carbonyl)piperazine-1-carboxylate formate (50 mg, 0.120 mmol, 15.07% yield, FA salt) as a brown solid.

RT 0.249 min (method 4); m/z 318.2 (M+H)⁺ (ESI⁺), ¹H NMR (DMSO-d₆, 400 MHz): 7.46-7.34 (m, 5H), 5.10 (s, 2H), 4.43-4.01 (m, 2H), 3.98-3.82 (m, 1H), 3.65-3.55 (m, 2H), 3.55-3.46 (m, 2H), 3.44-3.38 (m, 4H), 3.14-3.07 (m, 2H), 2.81 (s, 3H).

### Preparation of Intermediate 281.2

### (1-methylazetidin-3-yl)(piperazin-1-yl)methanone

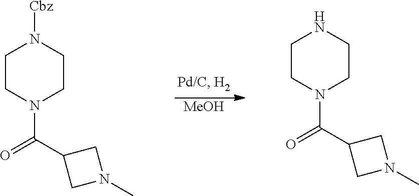 [SMILES: "MeOH" ; CN1CC(C(=O)N2CCN(C(=O)OCc3ccccc3)CC2)C1 ; CN1CC(C(=O)N2CCNCC2)C1]

To a suspension of Pd/C (17 mg, 10% purity) in MeOH (2 mL) was added benzyl 4-(1-methylazetidine-3-carbonyl)piperazine-1-carboxylate formate (50 mg, 0.137 mmol, FA salt). The suspension was degassed under vacuum and purged with H₂ (3×) before the mixture was stirred at 25° C. for 4 h under a H₂ atmosphere. The suspension was filtered through a pad of celite, and the cake was washed by MeOH (3 mL, 4×). The combined filtrates were concentrated under vacuum to give the crude product (1-methylazetidin-3-yl)(piperazin-1-yl)methanone (38 mg, 0.124 mmol, 90.78% yield) as a colorless oil which was used in the next step without further purification.

RT 0.060 min (method 4); m/z 184.2 (M+H)⁺ (ESI⁺)

### Preparation of Example 281

### 3-(5-(difluoromethyl)-1,3,4-thiadiazol-2-yl)-8-(4-(1-methylazetidine-3-carbonyl)piperazin-1-yl)-N-(1-methylcyclopropyl)imidazo[1,5-a]pyridine-6-sulfonamide formate

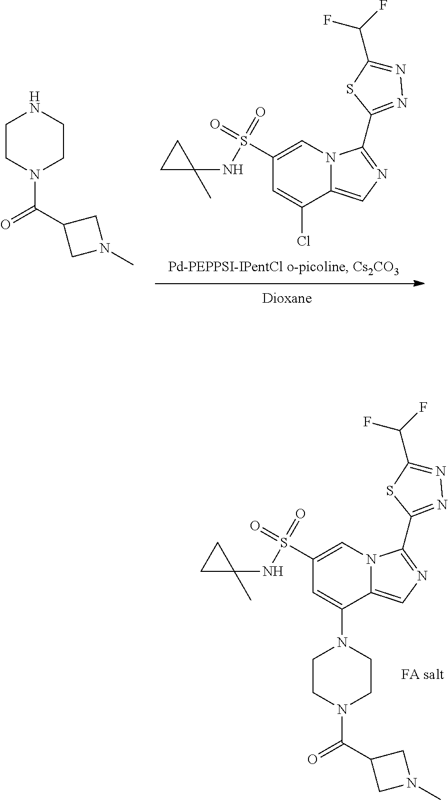 [SMILES: CN1CC(C(=O)N2CCNCC2)C1 ; CC1(NS(=O)(=O)c2cc(Cl)c3cnc(-c4nnc(C(F)F)s4)n3c2)CC1 ; CN1CC(C(=O)N2CCN(c3cc(S(=O)(=O)NC4(C)CC4)cn4c(-c5nnc(C(F)F)s5)ncc34)CC2)C1]

To a solution of 8-chloro-3-(5-(difluoromethyl)-1,3,4-thiadiazol-2-yl)-N-(1-methylcyclopropyl)imidazo[1,5-a]pyridine-6-sulfonamide (30.0 mg, 0.0715 mmol) in dioxane (1 mL) was added (1-methylazetidin-3-yl)(piperazin-1-yl)methanone (33.0 mg, 0.179 mmol), Cs₂CO₃ (70 mg, 0.21 mmol) and Pd-PEPPSI-IPentCl o-picoline (7.0 mg, 0.00715 mmol). The mixture was degassed and purged with N₂ (3×) before the mixture was stirred at 100° C. for 16 h under a N₂ atmosphere. The reaction mixture was cooled to 25° C. and filtered. The filtrate was concentrated under vacuum to give a residue, which was purified by preparative HPLC (column: Unisil 3-100 C18 Ultra 150*25 mm*10 μm; mobile phase: A: 0.225% formic acid in water, B: MeCN; B %: 13%-43%, 10 min) and lyophilized directly to give the product 3-(5-(difluoromethyl)-1,3,4-thiadiazol-2-yl)-8-(4-(1-methylazetidine-3-carbonyl)piperazin-1-yl)-N-(1-methylcyclopropyl)imidazo[1,5-a]pyridine-6-sulfonamide formate (12.94 mg, 0.022 mmol, 28.61% yield, 96.78% purity, FA salt) as a yellow solid.

RT 0.337 min (method 4); m/z 567.2 (M+H)⁺ (ESI⁺), ¹H NMR (DMSO-d₆, 400 MHz): 9.60 (s, 1H), 8.44 (br, 1H), 8.21 (s, 1H), 8.05 (s, 1H), 7.67 (t, J=53.2 Hz, 1H), 6.68 (s, 1H), 3.75-3.71 (m, 2H), 3.55-3.54 (m, 3H), 3.55-3.54 (m, 2H), 3.52-3.31 (m, 2H), 3.30-3.25 (m, 4H), 2.29 (s, 3H), 1.15 (s, 3H), 0.75-0.68 (m, 2H), 0.47-0.41 (m, 2H).

### Preparation of Example 282a

### tert-butyl (R)-4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-2-methylpiperazine-1-carboxylate

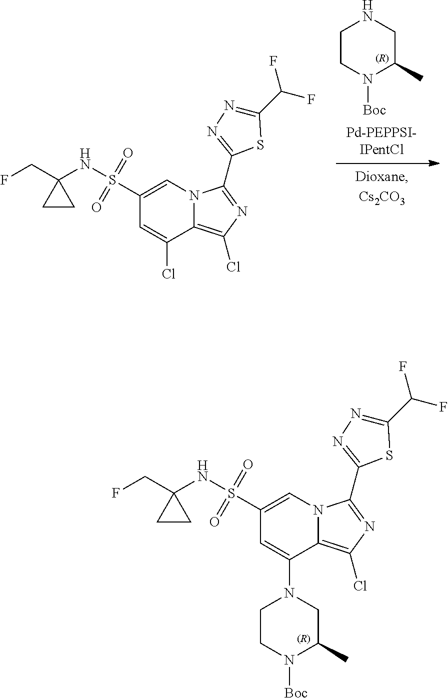

To a solution of 1,8-dichloro-3-[5-(difluoromethyl)-1,3,4-thiadiazol-2-yl]-N-[1-(fluoromethyl)cyclopropyl]imidazo[1,5-a]pyridine-6-sulfonamide (50.0 mg, 0.106 mmol) in dioxane (1 mL) was added tert-butyl rac-(2R)-2-methylpiperazine-1-carboxylate (42.0 mg, 0.212 mmol) followed by Cs₂CO₃ (103 mg, 0.318 mmol) and Pd-PEPPSI-IPentCl (10.0 mg, 0.0106 mmol). The mixture was degassed and purged with N₂ (3×) before it was stirred at 100° C. for 1 h. The resulting mixture was cooled to 20° C., diluted with EtOAc (10 mL) and filtered. The filtrate was concentrated in vacuo to give a residue which was purified by preparative TLC to afford the product tert-butyl (R)-4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-2-methylpiperazine-1-carboxylate (10.0 mg, 0.0138 mmol, 13.05% yield, 87.91% purity) as a yellow solid.

RT 0.855 min (method 2); m/z 636.0 (M+H)⁺ (ESI⁺); ¹H NMR (CDCl₃, 400 MHz): 9.90 (s, 1H), 7.08 (t, J=53.6 Hz, 1H), 6.71 (s, 1H), 5.59 (s, 1H), 4.45-4.57 (m, 1H), 4.28 (d, J=48.4 Hz, 2H), 4.02-3.94 (m, 1H), 3.65-3.53 (m, 2H), 3.19-3.07 (m, 2H), 2.70-2.58 (m, 1H), 1.51 (s, 9H), 1.39 (d, J=6.8 Hz, 3H), 1.19-1.14 (m, 2H), 0.91-0.87 (m, 2H).

### Preparation of Example 282

### (R)-1-chloro-3-(5-(difluoromethyl)-1,3,4-thiadiazol-2-yl)-N-(1-(fluoromethyl)cyclopropyl)-8-(3-methylpiperazin-1-yl)imidazo[1,5-a]pyridine-6-sulfonamide bis(2,2,2-trifluoroacetate)

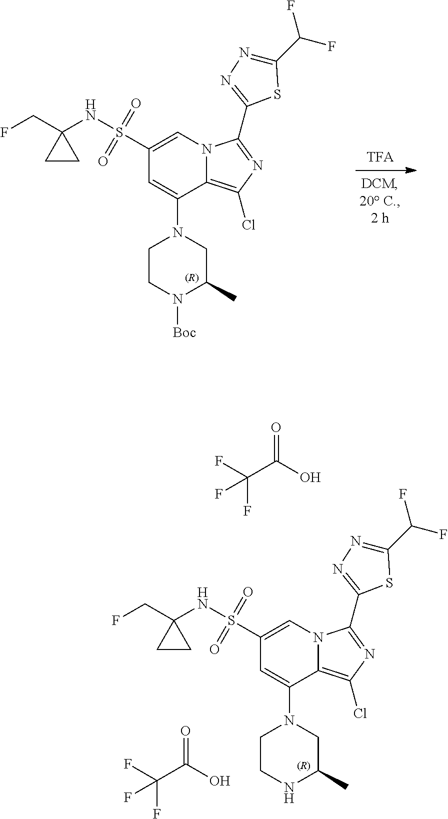

To a solution of tert-butyl (R)-4-(1-chloro-3-(5-(difluoromethyl)-1,3,4-thiadiazol-2-yl)-6-(N-(1-(fluoromethyl)cyclopropyl)sulfamoyl)imidazo[1,5-a]pyridin-8-yl)-2-methylpiperazine-1-carboxylate (10.0 mg, 0.0157 mmol) in DCM (1 mL) was added TFA (0.20 mL, 2.63 mmol) and the mixture was stirred at 22° C. for 1 h. The mixture was concentrated in vacuo at 32° C. The residue was dissolved in MeCN (2 mL), filtered and the filtrate was purified by preparative HPLC (column: Welch Xtimate C18 150*25 mm*5 μm; mobile phase: A: 0.075% TFA in water, B: MeCN; B %: 20%-50%, 8 min) and lyophilized directly to give the product (R)-1-chloro-3-(5-(difluoromethyl)-1,3,4-thiadiazol-2-yl)-N-(1-(fluoromethyl)cyclopropyl)-8-(3-methylpiperazin-1-yl)imidazo[1,5-a]pyridine-6-sulfonamide bis(2,2,2-trifluoroacetate) (4.3 mg, 0.0056 mmol, 35.69% yield, 99.46% purity, 2TFA salt) as a yellow solid.

RT 0.370 min (method 1); m/z 536.2 (M+H)⁺ (ESI⁺); ¹H NMR (400 MHz, DMSO-d₆): 9.62 (s, 1H), 9.21-9.02 (m, 1H), 8.90 (s, 1H), 8.79-8.65 (m, 1H), 7.69 (t, J=52.8 Hz, 1H), 6.94-6.87 (m, 1H), 4.23 (t, J=48.4, 2H), 3.57-3.51 (m, 5H), 3.07-2.98 (m, 1H), 2.89-2.81 (m, 1H), 1.30 (d, J=6.4 Hz, 3H), 0.90-0.85 (m, 2H), 0.82-0.76 (m, 2H).

### Preparation of Example 283.1

### tert-butyl (2R,6R)-4-(3-(5-(difluoromethyl)-1,3,4-thiadiazol-2-yl)-6-(N-(1-methylcyclopropyl)sulfamoyl)imidazo[1,5-a]pyridin-8-yl)-2,6-dimethylpiperazine-1-carboxylate

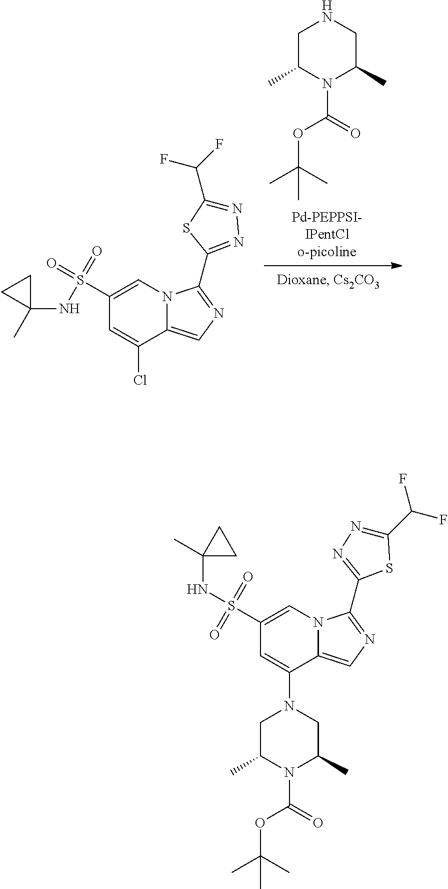

To a mixture of 8-chloro-3-(5-(difluoromethyl)-1,3,4-thiadiazol-2-yl)-N-(1-methylcyclopropyl)imidazo[1,5-a]pyridine-6-sulfonamide (50.0 mg, 0.119 mmol) and tert-butyl (2R,6R)-2,6-dimethylpiperazine-1-carboxylate (51.0 mg, 0.238 mmol) in dioxane (1 mL) were added Cs₂CO₃ (116 mg, 0.357 mmol) and Pd-PEPPSI-IPentCl o-picoline (12.0 mg, 0.0119 mmol). The mixture was degassed and purged with N₂ (3×) before it was stirred at 100° C. for 4 h under a N₂ atmosphere. The reaction mixture was filtered and the filtrate was concentrated under vacuum. The residue was purified by preparative TLC (petroleum ether:ethyl acetate=2:1) to give the product tert-butyl (2R,6R)-4-(3-(5-(difluoromethyl)-1,3,4-thiadiazol-2-yl)-6-(N-(1-methylcyclopropyl)sulfamoyl)imidazo[1,5-a]pyridin-8-yl)-2,6-dimethylpiperazine-1-carboxylate (15.0 mg, 0.0246 mmol, 20.65% yield) as a yellow solid.

RT 0.498 min (method 4); m/z 598.1 (M+H)⁺ (ESI⁺);

### Preparation of Example 283

### 3-(5-(difluoromethyl)-1,3,4-thiadiazol-2-yl)-8-((3R,5R)-3,5-dimethylpiperazin-1-yl)-N-(1-methylcyclopropyl)imidazo[1,5-a]pyridine-6-sulfonamide formate

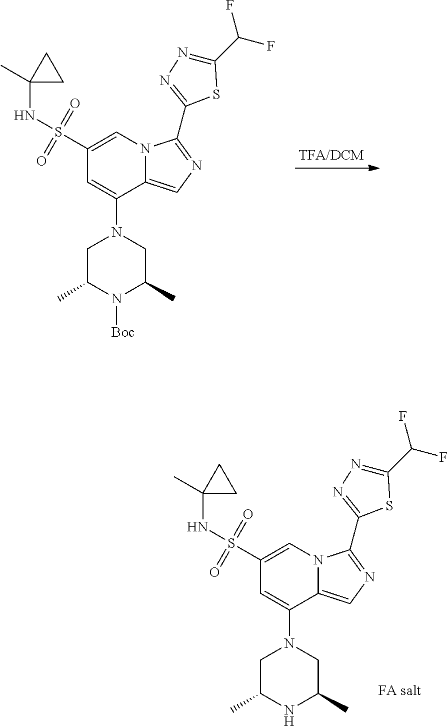 [SMILES: C[C@@H]1CN(c2cc(S(=O)(=O)NC3(C)CC3)cn3c(-c4nnc(C(F)F)s4)ncc23)C[C@@H](C)N1C(=O)OC(C)(C)C ; C[C@@H]1CN(c2cc(S(=O)(=O)NC3(C)CC3)cn3c(-c4nnc(C(F)F)s4)ncc23)C[C@@H](C)N1]

To a solution of tert-butyl (2R,6R)-4-(3-(5-(difluoromethyl)-1,3,4-thiadiazol-2-yl)-6-(N-(1-methylcyclopropyl)sulfamoyl)imidazo[1,5-a]pyridin-8-yl)-2,6-dimethylpiperazine-1-carboxylate (15.0 mg, 0.0246 mmol) in DCM (1 mL) was added TFA (0.2 mL) at 20° C., and the mixture was stirred at 20° C. for 2 h. The mixture was concentrated undervacuum. The residue was purified by preparative HPLC (column: Phenomenex luna C18 150*25 mm*10 μm; mobile phase: A: 0.225% formic acid in water, B: MeCN; B %: 13%-43%, 10 min) and lyophilized to give the product 3-(5-(difluoromethyl)-1,3,4-thiadiazol-2-yl)-8-((3R,5R)-3,5-dimethylpiperazin-1-yl)-N-(1-methylcyclopropyl)imidazo[1,5-a]pyridine-6-sulfonamide formate (1.6 mg, 0.0028 mmol, 11.52% yield, FA salt) as a yellow solid.

RT 0.333 min (method 4); m/z 498.1 (M+H)⁺ (ESI⁺); ¹H NMR (CDCl₃, 400 MHz): 9.88 (s, 1H), 8.47 (s, 1H), 7.70 (s, 1H), 7.09 (t, J=53.6 Hz, 1H), 6.71 (s, 1H), 5.49 (br s, 1H), 3.60-3.72 (m, 2H), 3.36-3.45 (m, 2H), 3.20-3.28 (m, 2H), 1.49 (d, J=6.0 Hz, 6H), 1.38 (s, 3H), 0.96-0.90 (m, 2H), 0.63-0.56 (m, 2H).

### Preparation of Intermediate 284.1

### tert-butyl (2S,6S)-4-(3-(5-(difluoromethyl)-1,3,4-thiadiazol-2-yl)-6-(N-(1-methylcyclopropyl)sulfamoyl)imidazo[1,5-a]pyridin-8-yl)-2,6-dimethylpiperazine-1-carboxylate

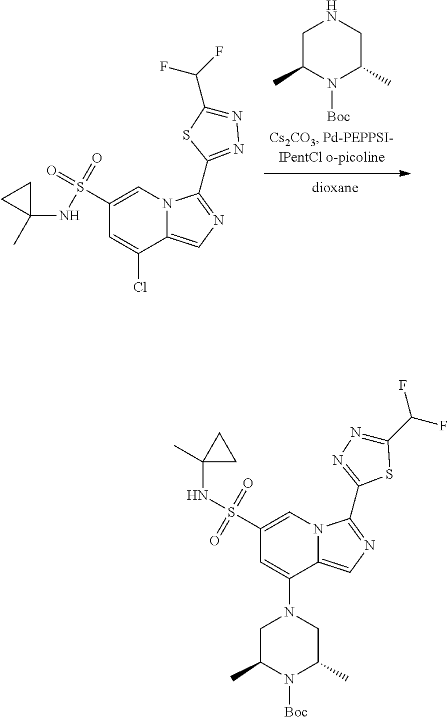

To a mixture of 8-chloro-3-(5-(difluoromethyl)-1,3,4-thiadiazol-2-yl)-N-(1-methylcyclopropyl)imidazo[1,5-a]pyridine-6-sulfonamide (30 mg, 0.0715 mmol) in dioxane (0.5 mL) was added tert-butyl (2S,6S)-2,6-dimethylpiperazine-1-carboxylate (15 mg, 0.0715 mmol), Cs₂CO₃ (70 mg, 0.214 mmol) and Pd-PEPPSI-IPentCl o-picoline (7.0 mg, 0.00715 mmol). The reaction mixture was degassed with N₂ (3×) and then stirred at 98° C. for 1 h. The mixture was filtered and the filtrate was concentrated under vacuum. The residue was purified by preparative TLC (Petroleum ether:Ethyl acetate=1:2) to give the product tert-butyl (2S,6S)-4-(3-(5-(difluoromethyl)-1,3,4-thiadiazol-2-yl)-6-(N-(1-methylcyclopropyl)sulfamoyl)imidazo[1,5-a]pyridin-8-yl)-2,6-dimethylpiperazine-1-carboxylate (16 mg, 0.0238 mmol, 33.34% yield) as a yellow solid.

RT 0.573 min (method 4); m/z 598.1 (M+H⁺) (ESI⁺); ¹H NMR (CDCl₃, 400 MHz): 9.74 (s, 1H), 7.83 (s, 1H), 7.08 (t, J=53.6 Hz, 1H), 6.36 (s, 1H), 5.06 (s, 1H), 4.20-4.37 (m, 2H), 4.14-4.11 (m, 2H), 3.67-3.48 (m, 2H), 1.52 (s, 9H), 1.40 (s, 3H), 1.34 (d, J=6.8 Hz, 6H), 0.98-0.96 (m, 2H), 0.63-0.58 (m, 2H)

### Preparation of Example 284

### 3-(5-(difluoromethyl)-1,3,4-thiadiazol-2-yl)-8-((3S,5S)-3,5-dimethylpiperazin-1-yl)-N-(1-methylcyclopropyl)imidazo[1,5-a]pyridine-6-sulfonamide formate

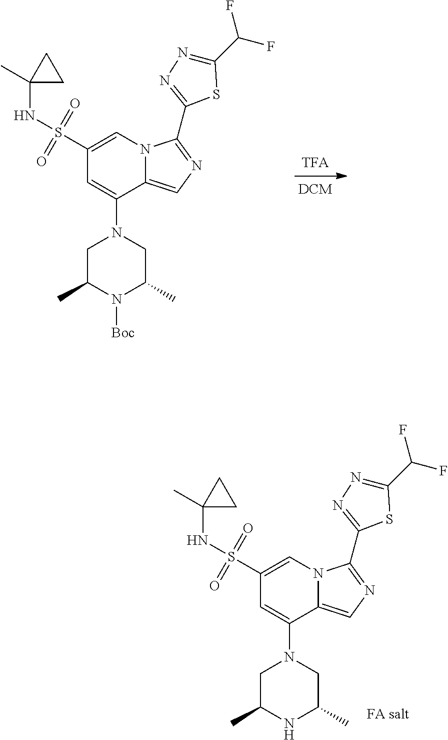 [SMILES: C[C@H]1CN(c2cc(S(=O)(=O)NC3(C)CC3)cn3c(-c4nnc(C(F)F)s4)ncc23)C[C@H](C)N1C(=O)OC(C)(C)C ; C[C@H]1CN(c2cc(S(=O)(=O)NC3(C)CC3)cn3c(-c4nnc(C(F)F)s4)ncc23)C[C@H](C)N1]

A solution of tert-butyl (2S,6S)-4-(3-(5-(difluoromethyl)-1,3,4-thiadiazol-2-yl)-6-(N-(1-methylcyclopropyl)sulfamoyl)imidazo[1,5-a]pyridin-8-yl)-2,6-dimethylpiperazine-1-carboxylate (16 mg, 0.0322 mmol) in DCM (0.5 mL) and TFA (0.1 mL) was stirred at 25° C. for 1 h. The mixture was concentrated under vacuum to give a residue, which was purified by preparative HPLC (column: Phenomenex luna C18 150*25 mm*10 μm; mobile phase: A: 0.225% formic acid in water, B: MeCN; B %: 14%-44%, 10 min) and lyophilized directly to give the product 3-(5-(difluoromethyl)-1,3,4-thiadiazol-2-yl)-8-((3S,5S)-3,5-dimethylpiperazin-1-yl)-N-(1-methylcyclopropyl)imidazo[1,5-a]pyridine-6-sulfonamide formate (4.0 mg, 0.00725 mmol, 22.56% yield, FA salt) as a yellow solid

RT 0.362 min (method 4); m/z 498.2 (M+H)⁺ (ESI⁺); ¹H NMR (DMSO-d₆, 400 MHz): 9.57 (s, 1H), 8.43 (s, 1H), 8.22 (s, 1H), 7.89 (s, 1H), 7.67 (t, J=53.2 Hz, 1H), 6.66 (s, 1H), 3.30-3.27 (m, 2H), 3.26-3.22 (m, 2H), 3.06-3.01 (m, 2H), 1.23 (d, J=6.4 Hz, 6H), 1.15 (s, 3H), 0.77-0.67 (m, 2H), 0.45-0.47 (m, 2H).

### Preparation of Intermediate 285.1

### 2-(6-bromo-8-chloroimidazo[1,5-a]pyridin-3-yl)-5-(difluoromethyl)-1,3,4-oxadiazole

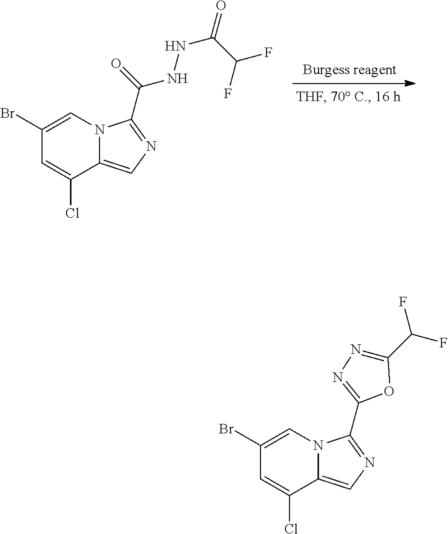 [SMILES: O=C(NNC(=O)C(F)F)c1ncc2c(Cl)cc(Br)cn12 ; FC(F)c1nnc(-c2ncc3c(Cl)cc(Br)cn23)o1]

To a solution of 6-bromo-8-chloro-N′-(2,2-difluoroacetyl)imidazo[1,5-a]pyridine-3-carbohydrazide (2000 mg, 5.44 mmol) in THE (3 mL) was added Burgess reagent (3890 mg, 16.3 mmol), and the reaction mixture was stirred at 65° C. for 16 h. The mixture was cooled to 20° C. and concentrated to give a residue, which was triturated with MeOH (5 mL) for 30 min. After filtration, the cake was collected and dried to give the product 2-(6-bromo-8-chloroimidazo[1,5-a]pyridin-3-yl)-5-(difluoromethyl)-1,3,4-oxadiazole (750 mg, 2.10 mmol, 38.53% yield) as a yellow solid.

RT 0.449 min (method 4); m/z 350.9 (M+H)⁺ (ESI⁺); H NMR (CDCl₃, 400 MHz): 9.52 (s, 1H), 7.93 (s, 1H), 7.28 (s, 1H), 7.10-6.85 (t, J=51.6 Hz, 1H).

### Preparation of Intermediate 285.2

### 2-(6-(benzylthio)-8-chloroimidazo[1,5-a]pyridin-3-yl)-5-(difluoromethyl)-1,3,4-oxadiazole

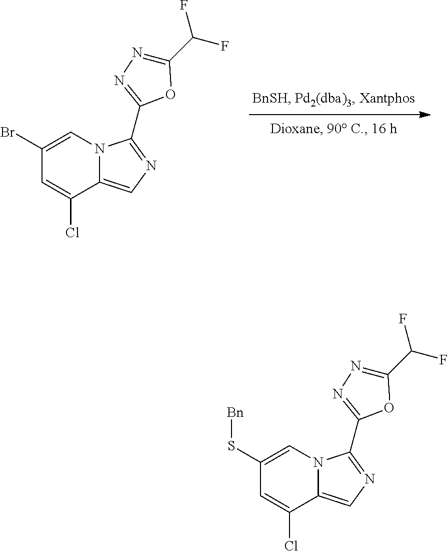 [SMILES: FC(F)c1nnc(-c2ncc3c(Cl)cc(Br)cn23)o1 ; FC(F)c1nnc(-c2ncc3c(Cl)cc(SCc4ccccc4)cn23)o1]

To a solution of 2-(6-bromo-8-chloroimidazo[1,5-a]pyridin-3-yl)-5-(difluoromethyl)-1,3,4-oxadiazole (1500 mg, 4.22 mmol) in 1,4-dioxane (15 mL) was added phenylmethanethiol (0.49 mL, 4.22 mmol), Pd₂(dba)₃ (386 mg, 0.422 mmol), Xantphos (244 mg, 0.422 mmol) and DIEA (2.2 mL, 12.6 mmol) at 25° C. before the reaction mixture was stirred at 100° C. for 1 h under a N₂ atmosphere. After cooling, the reaction mixture was diluted with DCM (20 mL) and filtered through a pad of silica gel. The filtrate was collected and concentrated under a reduced pressure to give a residue which was purified by preparative HPLC (Phenomenex luna C18 150*25 mm*10 um; mobile phase: 0.225% formic acid in water; B: 61%-91%, 10 min) to give the product 2-(6-(benzylthio)-8-chloroimidazo[1,5-a]pyridin-3-yl)-5-(difluoromethyl)-1,3,4-oxadiazole (710 mg, 1.81 mmol, 42.88% yield) as a white solid.

RT 0.483 min (method 4); m/z 393.1 (M+H)⁺ (ESI⁺); ¹H NMR (CDCl₃, 400 MHz) δ 9.17 (s, 1H), 7.85 (s, 1H), 7.37-7.34 (m, 2H), 7.34-7.28 (m, 2H), 7.26-7.18 (m, 1H), 7.03 (s, 1H), 6.96 (t, J=51.6 Hz, 1H), 4.19 (s, 2H).

### Preparation of Intermediate 285.3

### 8-chloro-3-(5-(difluoromethyl)-1,3,4-oxadiazol-2-yl)-N-(1-(fluoromethyl)cyclopropyl)imidazo[1,5-a]pyridine-6-sulfonamide

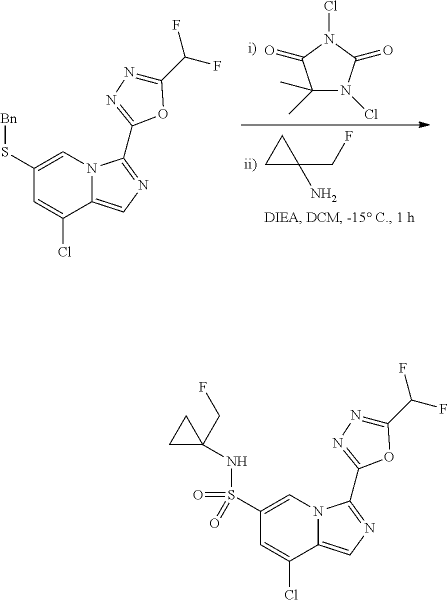

To a solution of 2-(6-benzylsulfanyl-8-chloro-imidazo[1,5-a]pyridin-3-yl)-5-(difluoromethyl)-1,3,4-oxadiazole (200 mg, 0.509 mmol) in AcOH (3.0 mL, 1.02 mmol) and H₂O (1.5 mL, 0.509 mmol) was added 1,3-dichloro-5,5-dimethylimidazolidine-2,4-dione (2.5 eq, 251 mg, 1.27 mmol) at 0° C. before the reaction mixture was stirred at 0° C. for 0.5 h. The mixture was quenched with H₂O (5 mL) at 0° C., and extracted with DCM (10 mL×3). The combined organic layer was dried over Na₂SO₄ and concentrated under a reduced pressure to give the crude product which was used in the next step without further purification.

To a solution of 1-(fluoromethyl)cyclopropanamine hydrochloride (102 mg, 0.81 mmol) in DCM (20 mL) was added dropwise DIEA (0.19 mL, 1.08 mmol) at −15° C. Then a solution of 8-chloro-3-[5-(difluoromethyl)-1,3,4-oxadiazol-2-yl]imidazo[1,5-a]pyridine-6-sulfonyl chloride (200 mg, 0.542 mmol) in DCM (5 mL) was added dropwise at −15° C. and the reaction mixture was stirred at −15° C. for 1 h. The reaction mixture was diluted with DCM (10 mL), and extracted with H₂O (10 mL×3) The combined organic layer was concentrated under reduced pressure to give a residue which was purified by preparative TLC (Petroleum ether:EtOAc=2:1) to give the product 8-chloro-3-[5-(difluoromethyl)-1,3,4-oxadiazol-2-yl]-N-[1-(fluoromethyl)cyclopropyl]imidazo[1,5-a]pyridine-6-sulfonamide (50 mg, 0.106 mmol, 19.48% yield) as a white solid.

RT 0.376 min (method 1); m/z 422.1 (M+H)⁺ (ESI⁺); ¹H NMR (CDCl₃, 400 MHz): 9.89 (s, 1H), 8.00 (s, 1H), 7.43 (s, 1H), 7.12 (t, J=51.2 Hz, 1H), 5.89 (s, 1H), 4.31 (d, J=48.4 Hz, 2H), 1.08-1.04 (m, 2H), 0.85-0.82 (m, 2H).

### Preparation of Example 285

### 4-(3-(5-(difluoromethyl)-1,3,4-oxadiazol-2-yl)-6-(N-(1-(fluoromethyl)cyclopropyl)sulfamoyl)imidazo[1,5-a]pyridin-8-yl)-N,N-dimethylpiperazine-1-carboxamide

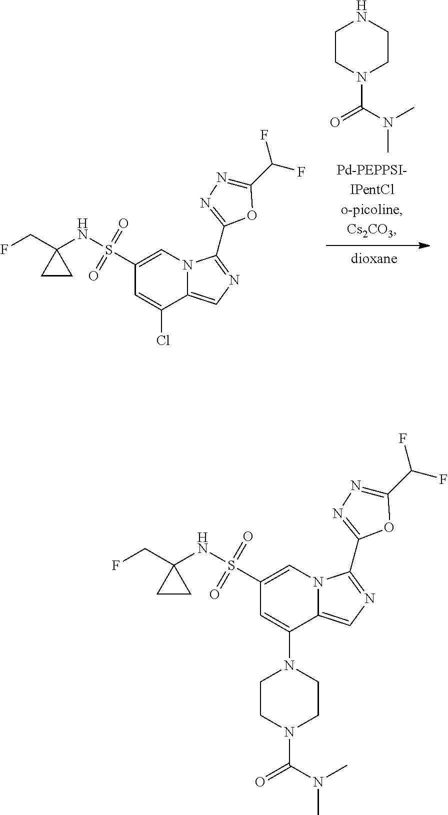 [SMILES: O=S(=O)(NC1(CF)CC1)c1cc(Cl)c2cnc(-c3nnc(C(F)F)o3)n2c1 ; CN(C)C(=O)N1CCNCC1 ; CN(C)C(=O)N1CCN(c2cc(S(=O)(=O)NC3(CF)CC3)cn3c(-c4nnc(C(F)F)o4)ncc23)CC1]

To a solution of 8-chloro-3-(5-(difluoromethyl)-1,3,4-oxadiazol-2-yl)-N-(1-(fluoromethyl)cyclopropyl)imidazo[1,5-a]pyridine-6-sulfonamide (20 mg, 0.047 mmol) in dioxane (1 mL) was added N,N-dimethylpiperazine-1-carboxamide (15 mg, 0.0948 mmol) followed by Cs₂CO₃ (46 mg, 0.142 mmol) and Pd-PEPPSI-IPentCl o-picoline (4.6 mg, 0.0047 mmol) at 25° C. in a glovebox. Outside of the glovebox, the reaction mixture was heated to 100° C. and stirred for 1.33 h. The mixture was cooled to 25° C., diluted with EtOAc (15 mL), filtered and the filter cake was washed with EtOAc (3 mL, 3×). The filtrate was concentrated in vacuo and the residue was purified by preparative TLC (SiO₂, Petroleum ether:Ethyl acetate=0:1) to give the product 4-(3-(5-(difluoromethyl)-1,3,4-oxadiazol-2-yl)-6-(N-(1-(fluoromethyl)cyclopropyl)sulfamoyl)imidazo[1,5-a]pyridin-8-yl)-N,N-dimethylpiperazine-1-carboxamide (1.2 mg, 0.00208 mmol, 4.38% yield) as a yellow solid.

RT 0.597 min (method 4); m/z 543.0 (M+H)⁺ (ESI⁺); ¹H NMR (CDCl₃, 400 MHz): 9.65 (s, 1H), 7.81 (s, 1H), 6.98 (t, J=51.6 Hz, 1H), 6.68 (s, 1H), 5.51 (s, 1H), 4.27 (d, J=48.4 Hz, 2H), 3.54-3.51 (m, 4H), 3.39-3.36 (m, 4H), 2.92 (s, 6H), 1.27-1.25 (m, 2H), 0.89-0.87 (m, 2H).

### Preparation of Intermediate 286.1

### tert-butyl (S)-4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-2-methylpiperazine-1-carboxylate

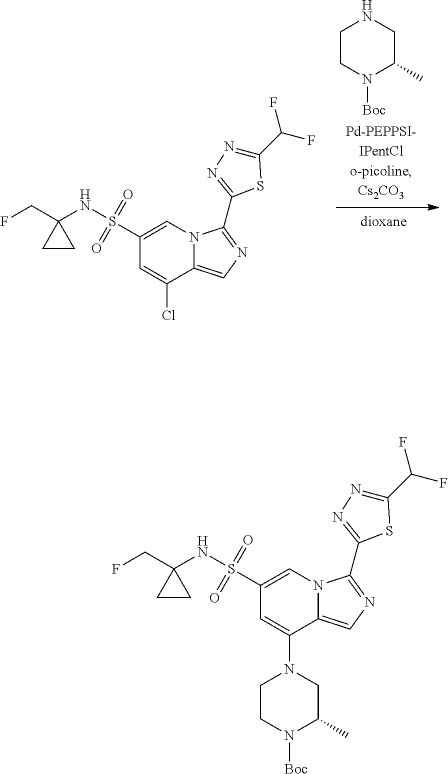 [SMILES: O=S(=O)(NC1(CF)CC1)c1cc(Cl)c2cnc(-c3nnc(C(F)F)s3)n2c1 ; C[C@H]1CNCCN1C(=O)OC(C)(C)C ; C[C@H]1CN(c2cc(S(=O)(=O)NC3(CF)CC3)cn3c(-c4nnc(C(F)F)s4)ncc23)CCN1C(=O)OC(C)(C)C]

To a solution of 8-chloro-3-(5-(difluoromethyl)-1,3,4-thiadiazol-2-yl)-N-(1-(fluoromethyl)cyclopropyl)imidazo[1,5-a]pyridine-6-sulfonamide (50 mg, 0.114 mmol) in dioxane (1 mL) was added tert-butyl (S)-2-methylpiperazine-1-carboxylate (46 mg, 0.228 mmol) followed by CS₂CO₃ (112 mg, 0.343 mmol) and Pd-PEPPSI-IPentCl o-picoline (11 mg, 0.011 mmol) at 22° C. The mixture was degassed and purged with N₂ (3×) before it was heated to 100° C. and stirred for 1 h. The resulting mixture was cooled to 22° C., diluted with EtOAc (10 mL), filtered and the filter cake was washed with EtOAc (3 mL, 3×). The combined filtrates were concentrated in vacuum. The residue was purified by preparative TLC (SiO₂, petroleum ether:Ethyl acetate=2:1) to give the product tert-butyl (S)-4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-2-methylpiperazine-1-carboxylate (42 mg, 0.0619 mmol, 54.18% yield, 88.63% purity) as a yellow solid.

RT 0.800 min (method 2); m/z 602.0 (M+H)⁺ (ESI⁺);

### Preparation of Example 286

### (S)-3-(5-(difluoromethyl)-1,3,4-thiadiazol-2-yl)-N-(1-(fluoromethyl)cyclopropyl)-8-(3-methylpiperazin-1-yl)imidazo[1,5-a]pyridine-6-sulfonamide

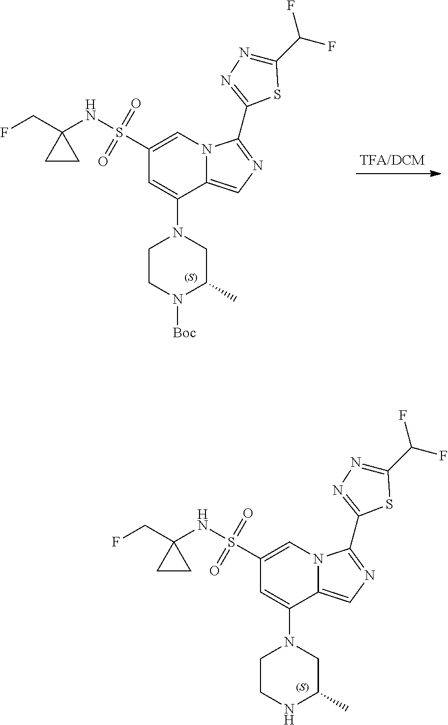

To a solution of tert-butyl (S)-4-(3-(5-(difluoromethyl)-1,3,4-thiadiazol-2-yl)-6-(N-(1-(fluoromethyl)cyclopropyl)sulfamoyl)imidazo[1,5-a]pyridin-8-yl)-2-methylpiperazine-1-carboxylate (42 mg, 0.070 mmol) in DCM (2 mL) was added TFA (0.4 mL) at 22° C. The reaction mixture was stirred at 22° C. for 0.5 h. The resulting mixture was concentrated in vacuo. The residue was purified by preparative HPLC (column: Welch Xtimate C18 150*25 mm*5 μm; mobile phase: A: 10 mmol NH₃·H₂O in water, B: MeCN; B %: 25%-55%, 10 min) and lyophilized directly to give the product (S)-3-(5-(difluoromethyl)-1,3,4-thiadiazol-2-yl)-N-(1-(fluoromethyl)cyclopropyl)-8-(3-methylpiperazin-1-yl)imidazo[1,5-a]pyridine-6-sulfonamide (9.5 mg, 0.0188 mmol, 26.97% yield, 99.7% purity) as a yellow solid.

RT 0.345 min (method 4); m/z 502.2 (M+H)⁺ (ESI⁺); ¹H NMR (DMSO-d₆, 400 MHz): 9.55 (s, 1H), 8.80 (br s, 1H), 7.98 (s, 1H), 7.67 (t, J=53.2 Hz, 1H), 6.68 (s, 1H), 4.22 (d, J=48.4 Hz, 2H), 3.63-3.51 (m, 2H), 3.03-2.92 (m, 3H), 2.82-2.75 (m, 1H), 2.48-2.44 (m, 1H), 1.06 (d, J=6.0 Hz, 3H), 0.88-0.81 (m, 2H), 0.80-0.74 (m, 2H).

### Preparation of Intermediate 287.1

### tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-(trifluoromethyl)piperazine-1-carboxylate

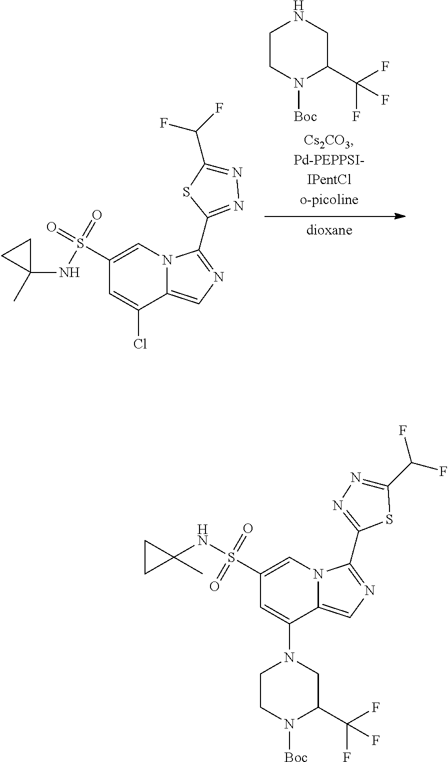 [SMILES: CC1(NS(=O)(=O)c2cc(Cl)c3cnc(-c4nnc(C(F)F)s4)n3c2)CC1 ; CC(C)(C)OC(=O)N1CCNCC1C(F)(F)F ; CC1(NS(=O)(=O)c2cc(N3CCN(C(=O)OC(C)(C)C)C(C(F)(F)F)C3)c3cnc(-c4nnc(C(F)F)s4)n3c2)CC1]

A mixture of 8-chloro-3-(5-(difluoromethyl)-1,3,4-thiadiazol-2-yl)-N-(1-methylcyclopropyl)imidazo[1,5-a]pyridine-6-sulfonamide (30 mg, 0.0715 mmol), tert-butyl 2-(trifluoromethyl)piperazine-1-carboxylate (36 mg, 0.143 mmol), Cs₂CO₃ (70 mg, 0.214 mmol) and Pd-PEPPSI-IPentCl o-picoline (7.0 mg, 0.00715 mmol) in dioxane (0.5 mL) was degassed with N₂ (3×) and stirred at 100° C. for 1 h. The resulting mixture was filtered and the filtrate was concentrated under vacuum. The residue was purified by preparative TLC (Petroleum ether:Ethyl acetate=2:1) to give the product tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-(trifluoromethyl)piperazine-1-carboxylate (10 mg, 0.0138 mmol, 19.30% yield) as a yellow solid.

RT 0.579 min (method 4); m/z 638.2 (M+H)⁺ (ESI⁺); ¹H NMR (CDCl₃, 400 MHz): 9.91 (s, 1H), 7.78 (s, 1H), 7.08 (t, J=53.6 Hz, 1H), 6.69 (s, 1H), 5.10 (br, 1H), 4.87-4.56 (m, 1H), 4.16-4.09 (m, 2H), 3.65-3.47 (m, 2H), 3.13-3.01 (m, 2H), 1.40 (s, 3H), 1.26 (s, 9H), 0.95-0.93-(m, 2H), 0.65-0.57 (m, 2H).

### Preparation of Example 287

### 3-(5-(difluoromethyl)-1,3,4-thiadiazol-2-yl)-N-(1-methylcyclopropyl)-8-(3-(trifluoromethyl)piperazin-1-yl)imidazo[1,5-a]pyridine-6-sulfonamide

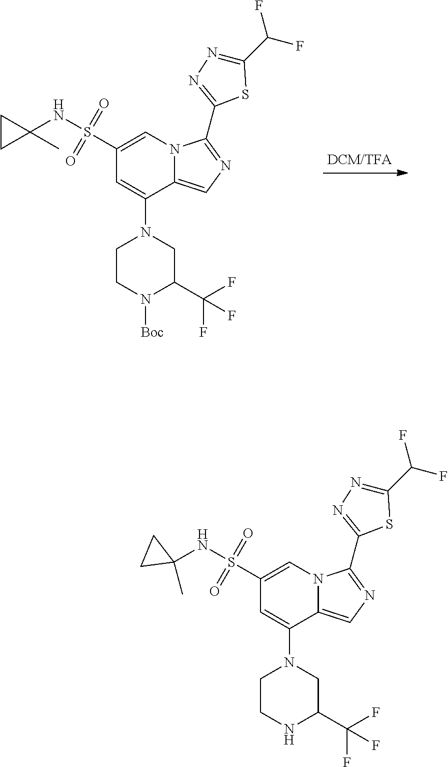 [SMILES: CC1(NS(=O)(=O)c2cc(N3CCN(C(=O)OC(C)(C)C)C(C(F)(F)F)C3)c3cnc(-c4nnc(C(F)F)s4)n3c2)CC1 ; CC1(NS(=O)(=O)c2cc(N3CCNC(C(F)(F)F)C3)c3cnc(-c4nnc(C(F)F)s4)n3c2)CC1]

A solution of tert-butyl 4-(3-(5-(difluoromethyl)-1,3,4-thiadiazol-2-yl)-6-(N-(1-methylcyclopropyl)sulfamoyl)imidazo[1,5-a]pyridin-8-yl)-2-(trifluoromethyl)piperazine-1-carboxylate (10 mg, 0.016 mmol) in DCM (0.2 mL) and TFA (0.04 mL) was stirred at 25° C. for 1 h. The mixture was concentrated under vacuum to give a residue, which was purified by preparative HPLC (column: Phenomenex luna C18 150*25 mm*10 μm; mobile phase: A: 0.225% formic acid in water, B: MeCN; B %: 34%-64%, 10 min) and lyophilized directly to give the product 3-(5-(difluoromethyl)-1,3,4-thiadiazol-2-yl)-N-(1-methylcyclopropyl)-8-(3-(trifluoromethyl)piperazin-1-yl)imidazo[1,5-a]pyridine-6-sulfonamide (0.60 mg, 0.001 mmol, 6.6% yield) as a yellow solid

RT 0.411 min (method 4); m/z 538.3 (M+H)⁺ (ESI⁺); ¹H NMR (DMSO-d₆, 400 MHz): 9.91 (s, 1H), 7.71 (s, 1H), 7.09 (t, J=53.2 Hz, 1H), 6.69 (s, 1H), 5.10 (s, 1H), 3.86-3.80 (m, 1H), 3.68-3.58 (m, 2H), 3.39-3.30 (m, 1H), 3.23-3.16 (m, 1H), 3.15-3.02 (m, 2H), 1.40 (s, 3H), 0.98-0.89 (m, 2H), 0.62-0.59 (m, 2H).

Compounds listed in the table below were prepared according to the corresponding general procedures or when stated in a similar way to related compound and starting from the corresponding intermediates

| Cpd | | Yield | | | |
|---|---|---|---|---|---|
| number | Structure | (%) | Procedure | LC/MS | ¹H NMR |
| 288 | 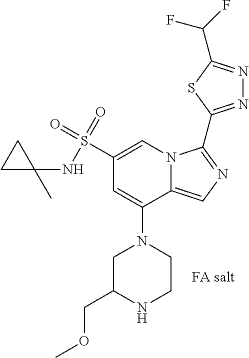 [SMILES: COCC1CN(c2cc(S(=O)(=O)NC3(C)CC3)cn3c(-c4nnc(C(F)F)s4)ncc23)CCN1] | 23.93% (Yield after 2 steps) | Procedure 1 then Procedure 2 | RT 0.359 min (method 4); m/z 514.2 (M + H)⁺ (ESI⁺) | ¹H NMR (D₂O, 400 MHz): 9.41 (s, 1H), 8.42 (s, 1H), 7.68 (s, 1H), 7.31 (t, J = 53.6 Hz, 1H), 6.66 (s, 1H), 3.78 (d, J = 3.2 Hz, 2H), 3.76-3.67 (m, 1H), 3.76- 3.75 (m, 2H), 3.60-3.51 (m, 1H), 3.45 (s, 3H), 3.39-3.31 (m, 1H), 3.31-3.14 (m, 2H), 1.17 (s, 3H), 0.81-0.71 (m, 2H), 0.56-0.46 (m, 2H). |
| 289 | 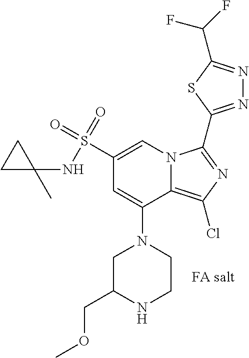 [SMILES: COCC1CN(c2cc(S(=O)(=O)NC3(C)CC3)cn3c(-c4nnc(C(F)F)s4)nc(Cl)c23)CCN1] | 11.32% (Yield after 2 steps) | Procedure 1 then Procedure 2 | RT 0.380 min (method 4); m/z 548.0 (M + H)⁺ (ESI⁺) | ¹H NMR (D₂O, 400 MHz): 9.56 (s, 1H), 8.42 (s, 1H), 7.33 (t, J = 53.6 Hz, 1H), 6.81 (s, 1H), 3.89-3.80 (m, 1H), 3.68-3.78 (m, 2H), 3.68-3.62 (m, 1H), 3.62- 3.53 (m, 2H), 3.52-3.46 (m, 1H), 3.45 (s, 3H), 3.26-2.97 (m, 2H), 1.19 (s, 3H), 0.73- 0.81 (m, 2H), 0.46-0.58 (m, 2H). |
| 290 | 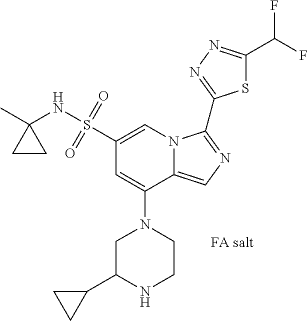 | 6.83% (Yield after 2 steps) | Procedure 1 then Procedure 2 | RT 0.372 min (method 4); m/z 510.1 (M + H)⁺ (ESI⁺) | ¹H NMR (DMSO-d₆, 400 MHz): 9.58 (s, 1H), 8.45 (br, 1H), 8.30 (br, 1H), 7.94 (s, 1H), 7.67 (t, J = 53.6 Hz, 1H), 6.68 (s, 1H), 3.55-3.65 (m, 2H), 3.01-3.07 (m, 1H), 2.88-2.93 (m, 1H), 2.86- 2.80 (m, 1H), 2.72-2.76 (m, 1H), 2.10-2.18 (m, 1H), 1.15 (s, 3H), 0.85-0.80 (m, 1H), 0.74-0.70 (m, 2H), 0.46-0.40 (m, 4H), 0.35- 0.34 (m, 2H). |
| 291 | 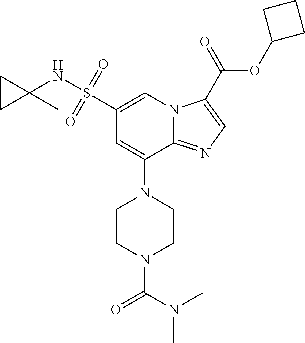 [SMILES: CN(C)C(=O)N1CCN(c2cc(S(=O)(=O)NC3(C)CC3)cn3c(C(=O)OC4CCC4)cnc23)CC1] | 21.25% | Procedure 14 | RT 0.545 min (method 3); m/z 505.2 (M + H)⁺ (ESI⁺) | ¹H NMR (CDCl₃, 400 MHz): 9.54 (s, 1H), 8.27 (s, 1H), 6.92 (s, 1H), 5.22-5.36 (m, 1H), 5.01 (s, 1H), 3.55-3.61 (m, 4H), 3.45-3.54 (m, 4H), 3.90 (s, 6H), 2.42-2.57 (m, 2H), 2.18-2.36 (m, 2H), 1.85-1.96 (m, 1H), 1.71- 1.80 (m, 1H), 1.32 (s, 3H), 0.85-0.87 (m, 2H), 0.54- 0.56 (m, 2H). |
| 292 | 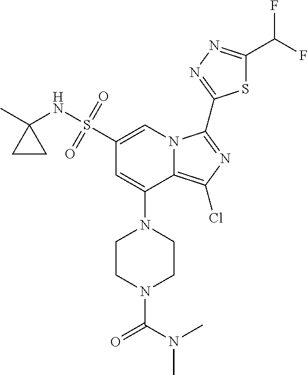 [SMILES: CN(C)C(=O)N1CCN(c2cc(S(=O)(=O)NC3(C)CC3)cn3c(-c4nnc(C(F)F)s4)nc(Cl)c23)CC1] | 1.94 | Procedure 1 | RT 0.507 min (method 4); m/z 575.0 (M + H)⁺ (ESI⁺) | ¹H NMR (CDCl₃, 400 MHz): 9.89 (s, 1H), 7.08 (t, J = 53.6 Hz, 1H), 6.74 (s, 1H), 5.10 (s, 1H), 3.48-3.61 (m, 4H), 3.26-3.18 (m, 4H), 2.90 (s, 6H), 1.39 (s, 3H), 0.97-0.87 (m, 2H), 0.64- 0.58 (m, 2H). |
| 293 | 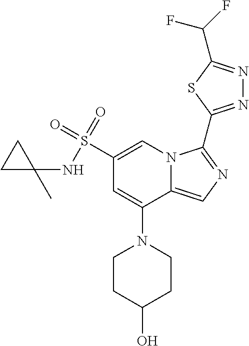 [SMILES: CC1(NS(=O)(=O)c2cc(N3CCC(O)CC3)c3cnc(-c4nnc(C(F)F)s4)n3c2)CC1] | 20.15 | Procedure 1 | RT 0.454 min (method 4); m/z 485.1 (M + H)⁺ (ESI⁺) | ¹H NMR (CDCl₃, 400 MHz): 9.84 (s, 1H), 7.71 (s, 1H), 7.08 (t, J = 53.6 Hz, 1H), 6.66 (s, 1H), 5.10 (br, 1H), 4.09-3.95 (m, 1H), 3.72- 3.54 (m, 2H), 3.23-3.07 (m, 2H), 2.20-2.09 (m, 2H), 1.91-1.79 (m, 2H), 1.38 (s, 3H), 0.98-0.90 (m, 2H), 0.63-0.56 (m, 2H). |
| 39 FA salt | 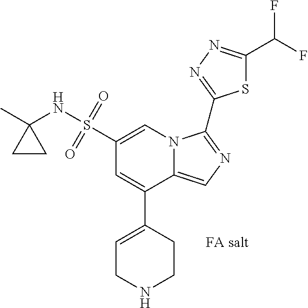 [SMILES: CC1(NS(=O)(=O)c2cc(C3=CCNCC3)c3cnc(-c4nnc(C(F)F)s4)n3c2)CC1] | 26.90 | Procedure 2 | RT 0.347 min (method 4); m/z 466.9 (M + H)⁺ (ESI⁺) | ¹H NMR (DMSO-d₆, 400 MHz): 9.85 (s, 1H), 8.50 (br, 1H), 8.23 (s, 1H), 8.11 (br, 1H), 7.69 (t, J = 53.2 Hz, 1H), 7.29 (s, 1H), 6.52 (s, 1H), 4.21-4.08 (m, 1H), 3.66-3.55 (m, 3H), 3.17- 3.07 (m, 2H), 1.17 (s, 3H), 0.78-0.69 (m, 2H), 0.51- 0.44 (m, 2H). |
| 295 | 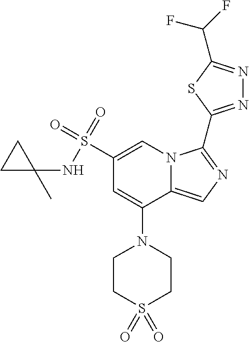 [SMILES: CC1(NS(=O)(=O)c2cc(N3CCS(=O)(=O)CC3)c3cnc(-c4nnc(C(F)F)s4)n3c2)CC1] | 18.32 | Procedure 1 | RT 0.438 min (method 4); m/z 519.0 (M + H)⁺ (ESI⁺) | ¹H NMR (CDCl₃, 400 MHz): 9.94 (s, 1H), 7.67 (s, 1H), 7.08 ( t, J = 53.6 Hz, 1H), 6.76 (s, 1H), 5.13 (s, 1H), 3.9-3.86 (m, 4H), 3.35-3.30 (m, 4H), 1.41 (s, 3H), 0.96- 0.90 (m, 2H), 0.65-0.62 (m, 2H). |
| 296 | 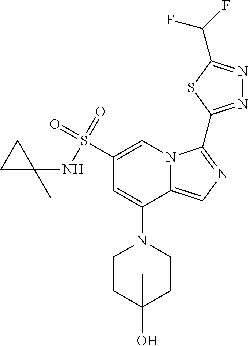 | 27.72 | Procedure 1 | RT 0.472 min (method 4); m/z 499.0 (M + H)⁺ (ESI⁺) | ¹H NMR (CDCl₃, 400 MHz): 9.83 (s, 1H), 7.71 (s, 1H), 7.08 (J = 53.6 Hz, 1H), 6.67 (s, 1H), 5.10 (br, 1H), 3.54- 3.47 (m, 2H), 3.40-3.30 (m, 2H), 1.99-1.89 (m, 2H), 1.80-1.75 (m, 2H), 1.40 (s, 3H), 1.38 (s, 3H), 0.96-0.92 (m, 2H), 0.61-0.57 (m, 2H). |
| 297 | 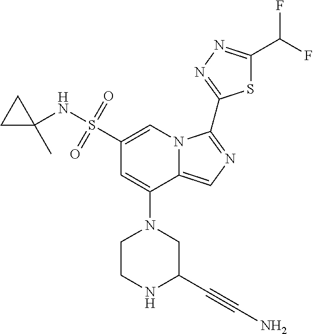 | 0.85 (Yield after 2 steps) | Procedure 1 then Procedure 2 | RT 0.362 min (method 4); m/z 495.2 (M + H)⁺ (ESI⁺) | ¹H NMR (CDCl₃, 400 MHz): 9.91 (s, 1H), 7.90 (s, 1H), 7.08 (t, J = 53.6 Hz, 1H), 6.71 (s, 1H), 5.10 (s, 1H), 4.24 (s, 1H), 3.83-3.93 (m, 1H), 3.60-3.39 (m, 2H), 3.30-3.11 (m, 3H), 1.40 (s, 3H), 0.97-0.91 (m, 2H), 0.66-0.59 (m, 2H). |
| 298 | 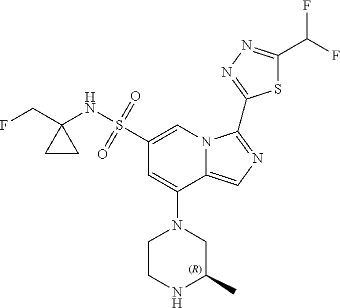 | 25.41 (Yield after 2 steps) | Procedure 1 then Procedure 2 | RT 0.348 min (method 4); m/z 502.3 (M + H)+ (ESI+); | ¹H NMR (DMSO-d₆, 400 MHz): 9.55 (s, 1H), 8.76 (br, 1H), 7.98 (s, 1H), 7.67 (t, J = 52.8 Hz, 1H), 6.65 (s, 1H), 4.22 (d, J = 48.8 Hz, 2H), 3.63-3.52 (m, 2H), 3.02-2.92 (m, 3H), 2.82- 2.75 (m, 1H), 2.48-2.44 (m, 1H), 1.06 (d, J = 6.4 Hz, 3H), 0.87-0.82 (m, 2H), 0.79-0.75 (m, 2H). |
| 299 | 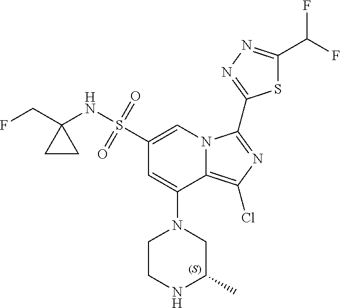 | 6.30 (Yield after 2 steps) | Procedure 1 then Procedure 2 | RT 0.369 min (method 4); m/z 536.2 (M + H)+ (ESI+); | ¹H NMR (DMSO-d₆, 400 MHz): 9.57 (s, 1H), 8.80 (br, 1H), 7.68 (t, J = 52.8 Hz, 1H), 6.79 (s, 1H), 4.22 (d, J = 48.8 Hz, 2H), 3.29- 3.26 (m, 2H), 3.03-2.96 (m, 3H), 2.70-2.62 (m, 1H), 2.37-2.32 (m, 1H), 1.03 (d, J = 6.4 Hz, 3H), 0.90-0.83 (m, 2H), 0.81-0.75 (m, 2H). |

The following Table 1 provides an overview on the compounds described in the example section:

TABLE 1

| Example | | |
|---|---|---|
| No. | Structure | Name of compound |
| 1 | 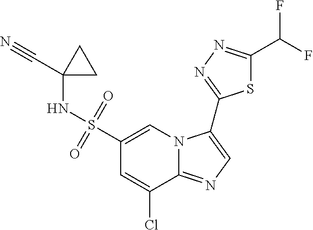 [SMILES: N#CC1(NS(=O)(=O)c2cc(Cl)c3ncc(-c4nnc(C(F)F)s4)n3c2)CC1] | 8-chloro-N-(1-cyanocyclopropyl)-3-(5- (difluoromethyl)- 1,3,4-thiadiazol-2-yl)imidazo[1,2-a]pyridine-6- sulfonamide |
| 2 | 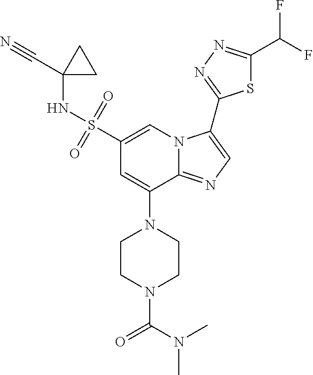 [SMILES: CN(C)C(=O)N1CCN(c2cc(S(=O)(=O)NC3(C#N)CC3)cn3c(-c4nnc(C(F)F)s4)cnc23)CC1] | 4-(6-(N-(1-cyanocyclopropyl)sulfamoyl)-3- (5-(difluoromethyl)-1,3,4-thiadiazol-2-yl) imidazo[1,2-a]pyridin-8-yl)-N,N- dimethylpiperazine-1- carboxamide |
| 3 | 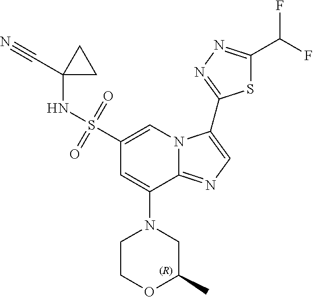 | (R)-N-(1-cyanocyclopropyl)-3-(5-(difluoromethyl)- 1,3,4-thiadiazol-2-yl)-8-(2- methylmorpholino)imidazo [1,2-a]pyridine-6-sulfonamide |
| 4 | 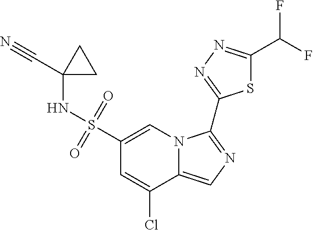 [SMILES: N#CC1(NS(=O)(=O)c2cc(Cl)c3cnc(-c4nnc(C(F)F)s4)n3c2)CC1] | 8-chloro-N-(1-cyanocyclopropyl)-3-(5- (difluoromethyl)- 1,3,4-thiadiazol-2-yl)imidazo[1,5-a]pyridine-6- sulfonamide |
| 5 | 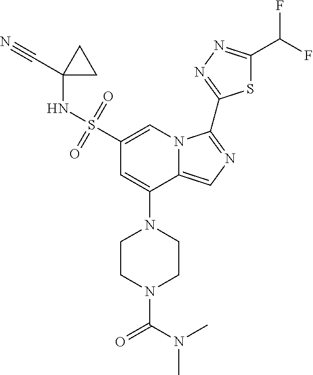 [SMILES: CN(C)C(=O)N1CCN(c2cc(S(=O)(=O)NC3(C#N)CC3)cn3c(-c4nnc(C(F)F)s4)ncc23)CC1] | 4-(6-(N-(1-cyanocyclopropyl)sulfamoyl)-3- (5-(difluoromethyl)-1,3,4-thiadiazol-2- yl)imidazo[1,5-a] pyridin-8-yl)-N,N-dimethylpiperazine-1- carboxamide |
| 6 | 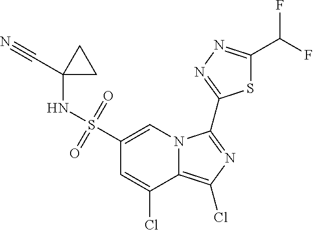 [SMILES: N#CC1(NS(=O)(=O)c2cc(Cl)c3c(Cl)nc(-c4nnc(C(F)F)s4)n3c2)CC1] | 1,8-dichloro-N-(1-cyanocyclopropyl)-3- (5-(difluoromethyl)-1,3,4-thiadiazol-2-yl) imidazo[1,5-a]pyridine-6-sulfonamide |
| 7 | 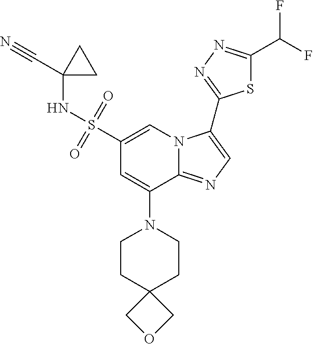 [SMILES: N#CC1(NS(=O)(=O)c2cc(N3CCC4(CC3)COC4)c3ncc(-c4nnc(C(F)F)s4)n3c2)CC1] | N-(1-cyanocyclopropyl)-3-(5-(difluoromethyl)- 1,3,4-thiadiazol-2-yl)-8-(2-oxa-7-azaspiro[3.5] nonan-7-yl)imidazo[1,2-a]pyridine-6-sulfonamide |
| 8 | 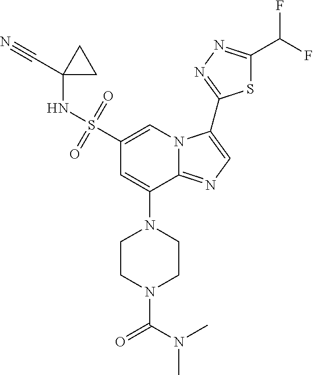 [SMILES: CN(C)C(=O)N1CCN(c2cc(S(=O)(=O)NC3(C#N)CC3)cn3c(-c4nnc(C(F)F)s4)cnc23)CC1] | N-(1-cyanocyclopropyl)-3-(5-(difluoromethyl)- 1,3,4-thiadiazol-2-yl)-8-(4-isobutyrylpiperazin-1- yl)imidazo[1,2-a]pyridine-6-sulfonamide |
| 9 | 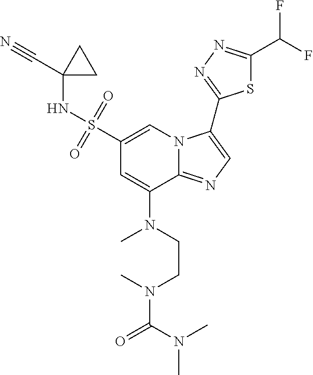 [SMILES: CN(C)C(=O)N(C)CCN(C)c1cc(S(=O)(=O)NC2(C#N)CC2)cn2c(-c3nnc(C(F)F)s3)cnc12] | N-(2-((6-(N-(1-cyanocyclopropyl)sulfamoyl)- 3-(5-(difluoromethyl)-1,3,4-thiadiazol-2-yl) imidazo[1,2-a]pyridin-8-yl)(methyl)amino)ethyl)- N-methylisobutyramide |
| 10 | 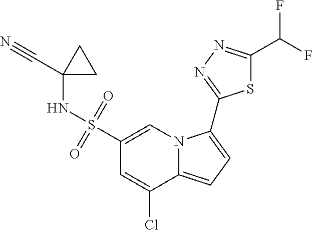 [SMILES: N#CC1(NS(=O)(=O)c2cc(Cl)c3ccc(-c4nnc(C(F)F)s4)n3c2)CC1] | [8-chloro-N-(1-cyanocyclopropyl)-3-(5- (difluoromethyl)-1,3,4-thiadiazol-2-yl)indolizine-6- sulfonamide |
| 11 | 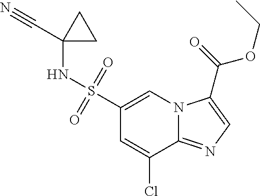 [SMILES: CCOC(=O)c1cnc2c(Cl)cc(S(=O)(=O)NC3(C#N)CC3)cn12] | ethyl 8-chloro-6-(N-(1- cyanocyclopropyl)sulfamoyl) imidazo[1,2-a]pyridine-3-carboxylate |
| 12 | 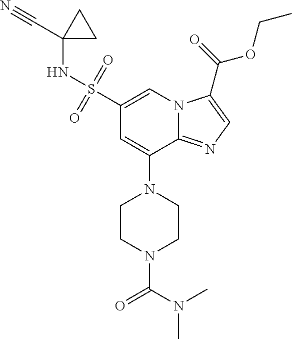 [SMILES: CCOC(=O)c1cnc2c(N3CCN(C(=O)N(C)C)CC3)cc(S(=O)(=O)NC3(C#N)CC3)cn12] | ethyl 6-(N-(1-cyanocyclopropyl)sulfamoyl)-8- (4-(dimethylcarbamoyl)piperazin-1-yl)imidazo [1,2-a]pyridine-3-carboxylate |
| 13 | 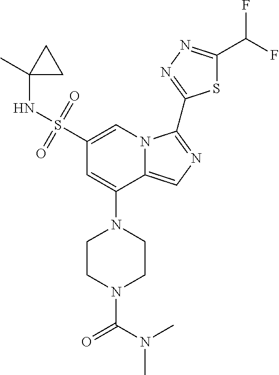 [SMILES: CN(C)C(=O)N1CCN(c2cc(S(=O)(=O)NC3(C)CC3)cn3c(-c4nnc(C(F)F)s4)ncc23)CC1] | 4-(3-(5-(difluoromethyl)-1,3,4-thiadiazol-2-yl)- 6-(N-(1-methylcyclopropyl)sulfamoyl)imidazo [1,5-a]pyridin-8-yl)-N,N-dimethylpiperazine-1- carboxamide |
| 14 | 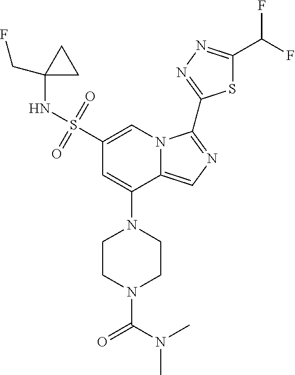 [SMILES: CN(C)C(=O)N1CCN(c2cc(S(=O)(=O)NC3(CF)CC3)cn3c(-c4nnc(C(F)F)s4)ncc23)CC1] | 4-(3-(5-(difluoromethyl)-1,3,4-thiadiazol-2-yl)- 6-(N-(1-(fluoromethyl)cyclopropyl)sulfamoyl) imidazo[1,5-a]pyridin-8-yl)-N,N- dimethylpiperazine- 1-carboxamide |
| 15 | 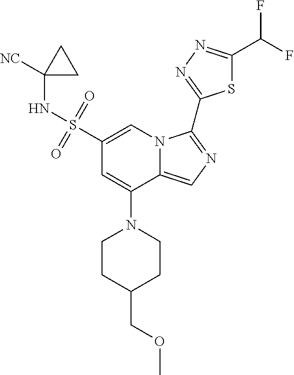 [SMILES: COCC1CCN(c2cc(S(=O)(=O)NC3(C#N)CC3)cn3c(-c4nnc(C(F)F)s4)ncc23)CC1] | N-(1-cyanocyclopropyl)-3-(5-(difluoromethyl)- 1,3,4-thiadiazol-2-yl)-8-(4- (methoxymethyl)piperidin-yl)imidazo[1,5- a]pyridine-6-sulfonamide |
| 16 | 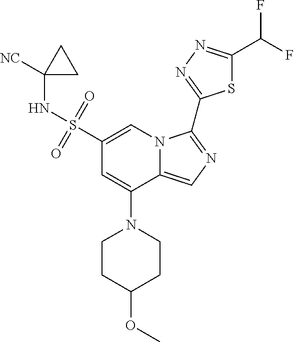 [SMILES: COC1CCN(c2cc(S(=O)(=O)NC3(C#N)CC3)cn3c(-c4nnc(C(F)F)s4)ncc23)CC1] | N-(1-cyanocyclopropyl)-3-(5- (difluoromethyl)-1,3,4-thiadiazol-2-yl)-8-(4- methoxypiperidin-1-yl)imidazo[1,5-a]pyridine-6- sulfonamide |
| 17 | 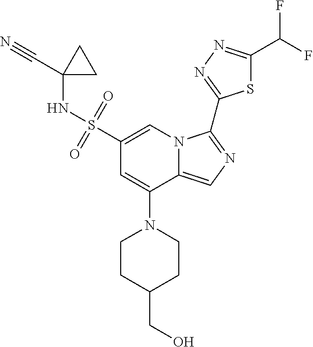 [SMILES: N#CC1(NS(=O)(=O)c2cc(N3CCC(CO)CC3)c3cnc(-c4nnc(C(F)F)s4)n3c2)CC1] | N-(1-cyanocyclopropyl)-3-(5- (difluoromethyl)-1,3,4-thiadiazol-2-yl)-8-(4- (hydroxymethyl)piperidin-1-yl)imidazo[1,5- a]pyridine-6-sulfonamide |
| 18a & 18b | 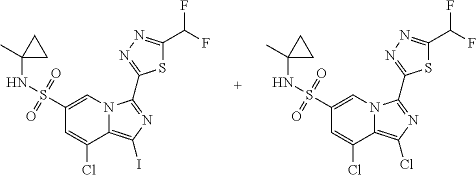 [SMILES: CC1(NS(=O)(=O)c2cc(Cl)c3c(I)nc(-c4nnc(C(F)F)s4)n3c2)CC1 ; CC1(NS(=O)(=O)c2cc(Cl)c3c(Cl)nc(-c4nnc(C(F)F)s4)n3c2)CC1] | 8-chloro-3-(5-(difluoromethyl)-1,3,4- thiadiazol-2-yl)-1-iodo-N-(1- methylcyclopropyl)imidazo[1,5-a]pyridine-6- sulfonamide and 1,8-dichloro-3-(5- (difluoromethyl)-1,3,4-thiadiazol-2-yl)-N-(1- methylcyclopropyl)imidazo[1,5-a]pyridine-6- sulfonamide |
| 18c | 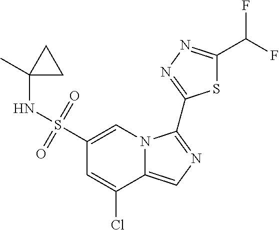 [SMILES: CC1(NS(=O)(=O)c2cc(Cl)c3cnc(-c4nnc(C(F)F)s4)n3c2)CC1] | 8-chloro-3-(5-(difluoromethyl)-1,3,4- thiadiazol-2-yl)-N-(1- methylcyclopropyl)imidazo[1,5-a]pyridine-6- sulfonamide |
| 18d | 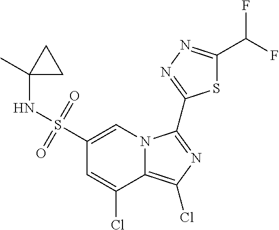 [SMILES: CC1(NS(=O)(=O)c2cc(Cl)c3c(Cl)nc(-c4nnc(C(F)F)s4)n3c2)CC1] | 1,8-dichloro-3-(5-(difluoromethyl)-1,3,4- thiadiazol-2-yl)-N-(1- methylcyclopropyl)imidazo[1,5-a]pyridine-6- sulfonamide |
| 18 | 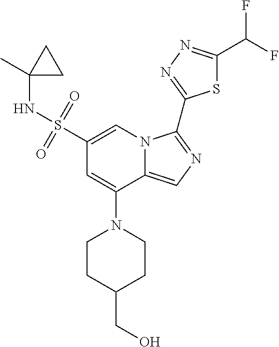 [SMILES: CC1(NS(=O)(=O)c2cc(N3CCC(CO)CC3)c3cnc(-c4nnc(C(F)F)s4)n3c2)CC1] | 3-(5-(difluoromethyl)-1,3,4-thiadiazol-2-yl)- 8-(4-(hydroxymethyl)piperidin-1-yl)-N-(1- methylcyclopropyl)imidazo[1,5-a]pyridine-6- sulfonamide |
| 19 | 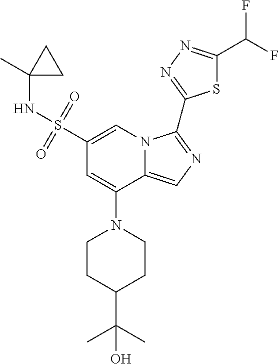 [SMILES: CC1(NS(=O)(=O)c2cc(N3CCC(C(C)(C)O)CC3)c3cnc(-c4nnc(C(F)F)s4)n3c2)CC1] | 3-(5-(difluoromethyl)-1,3,4-thiadiazol-2-yl)- 8-(4-(2-hydroxypropan-2-yl)piperidin-1-yl)-N-(1- methylcyclopropyl)imidazo[1,5-a]pyridine-6- sulfonamide |
| 20 | 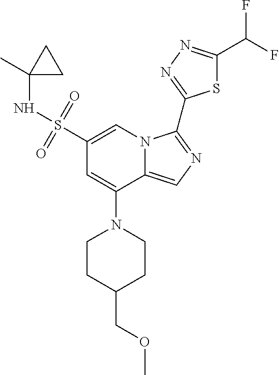 [SMILES: COCC1CCN(c2cc(S(=O)(=O)NC3(C)CC3)cn3c(-c4nnc(C(F)F)s4)ncc23)CC1] | 3-(5-(difluoromethyl)-1,3,4-thiadiazol-2-yl)- 8-(4-(methoxymethyl)piperidin-1-yl)-N-(1- methylcyclopropyl)imidazo[1,5-a]pyridine-6- sulfonamide |
| 21 | 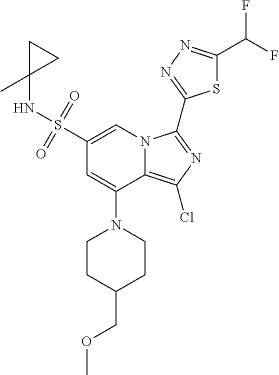 [SMILES: COCC1CCN(c2cc(S(=O)(=O)NC3(C)CC3)cn3c(-c4nnc(C(F)F)s4)nc(Cl)c23)CC1] | 1-chloro-3-(5-(difluoromethyl)-1,3,4- thiadiazol-2-yl)-8-(4-(methoxymethyl)piperidin-1- yl)-N-(1-methylcyclopropyl)imidazo[1,5- a]pyridine-6-sulfonamide |
| 22 | 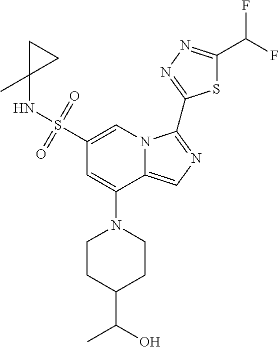 [SMILES: CC(O)C1CCN(c2cc(S(=O)(=O)NC3(C)CC3)cn3c(-c4nnc(C(F)F)s4)ncc23)CC1] | 3-(5-(difluoromethyl)-1,3,4-thiadiazol-2-yl)- 8-(4-(1-hydroxyethyl)piperidin-1-yl)-N-(1- methylcyclopropyl)imidazo[1,5-a]pyridine-6- sulfonamide |
| 23 | 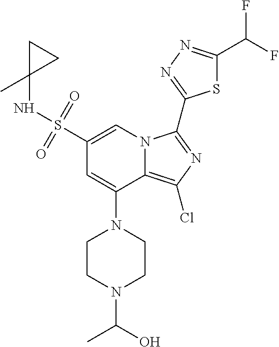 [SMILES: CC(O)N1CCN(c2cc(S(=O)(=O)NC3(C)CC3)cn3c(-c4nnc(C(F)F)s4)nc(Cl)c23)CC1] | Chloro-3-(5-(difluoromethyl)-1,3,4- thiadiazol-2-yl)-8-(4-(1-hydroxyethyl)piperidin-1- yl)-N-(1-methylcyclopropyl)imidazo[1,5- a]pyridine-6-sulfonamide |
| 24 | 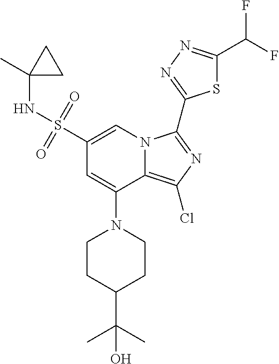 [SMILES: CC1(NS(=O)(=O)c2cc(N3CCC(C(C)(C)O)CC3)c3c(Cl)nc(-c4nnc(C(F)F)s4)n3c2)CC1] | 1-chloro-3-(5-(difluoromethyl)-1,3,4- thiadiazol-2-yl)-8-(4-(2-hydroxypropan-2- yl)piperidin-1-yl)-N-(1- methylcyclopropyl)imidazo[1,5-a]pyridine-6- sulfonamide |
| 25a | 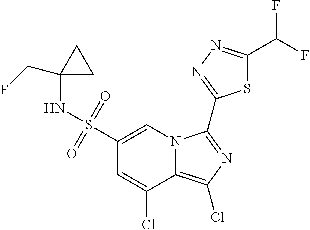 [SMILES: O=S(=O)(NC1(CF)CC1)c1cc(Cl)c2c(Cl)nc(-c3nnc(C(F)F)s3)n2c1] | 1,8-dichloro-3-(5-(difluoromethyl)-1,3,4- thiadiazol-2-yl)-N-(1- (fluoromethyl)cyclopropyl)imidazo[1,5-a]pyridine- 6-sulfonamide |
| 25 | 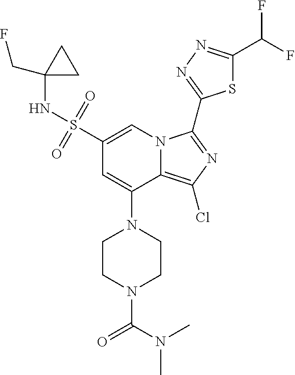 [SMILES: CN(C)C(=O)N1CCN(c2cc(S(=O)(=O)NC3(CF)CC3)cn3c(-c4nnc(C(F)F)s4)nc(Cl)c23)CC1] | 4-(1-chloro-3-(5-(difluoromethyl)-1,3,4- thiadiazol-2-yl)-6-(N-(1- (fluoromethyl)cyclopropyl)sulfamoyl)imidazo[1,5- a]pyridin-8-yl)-N,N-dimethylpiperazine-1- carboxamide |
| 26 | 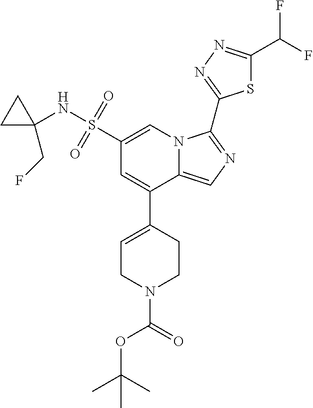 [SMILES: CC(C)(C)OC(=O)N1CC=C(c2cc(S(=O)(=O)NC3(CF)CC3)cn3c(-c4nnc(C(F)F)s4)ncc23)CC1] | tert-butyl 4-(3-(5-(difluoromethyl)-1,3,4- thiadiazol-2-yl)-6-(N-(1- (fluoromethyl)cyclopropyl)sulfamoyl)imidazo[1,5- a]pyridin-8-yl)-3,6-dihydropyridine-1(2H)- carboxylate |
| 27 | 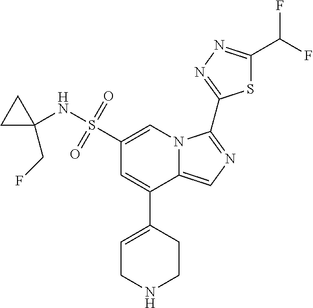 [SMILES: O=S(=O)(NC1(CF)CC1)c1cc(C2=CCNCC2)c2cnc(-c3nnc(C(F)F)s3)n2c1] | 3-(5-(difluoromethyl)-1,3,4-thiadiazol-2-yl)- N-(1-(fluoromethyl)cyclopropyl)-8-(1,2,3,6- tetrahydropyridin-4-yl)imidazo[1,5-a]pyridine-6- sulfonamide |
| 28 | 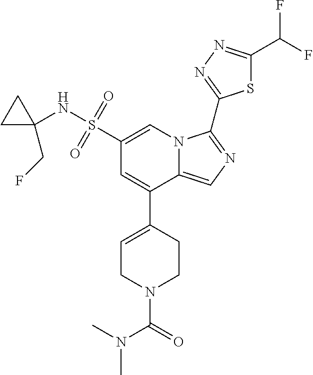 [SMILES: CN(C)C(=O)N1CC=C(c2cc(S(=O)(=O)NC3(CF)CC3)cn3c(-c4nnc(C(F)F)s4)ncc23)CC1] | -(3-(5-(difluoromethyl)-1,3,4-thiadiazol-2- yl)-6-(N-(1- (fluoromethyl)cyclopropyl)sulfamoyl)imidazo [1,5- a]pyridin-8-yl)-N,N-dimethyl-3,6-dihydropyridine- 1(2H)-carboxamide |
| 29 | 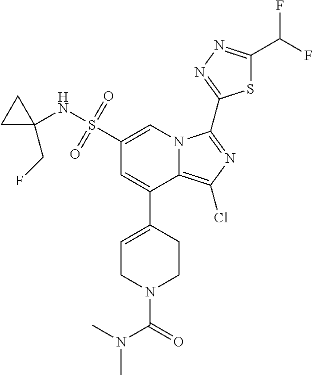 [SMILES: CN(C)C(=O)N1CC=C(c2cc(S(=O)(=O)NC3(CF)CC3)cn3c(-c4nnc(C(F)F)s4)nc(Cl)c23)CC1] | 4-(1-chloro-3-(5-(difluoromethyl)-1,3,4- thiadiazol-2-yl)-6-(N-(1- (fluoromethyl)cyclopropyl)sulfamoyl)imidazo[1,5- a]pyridin-8-yl)-N,N-dimethyl-3,6-dihydropyridine- 1(2H)-carboxamide |
| 30 | 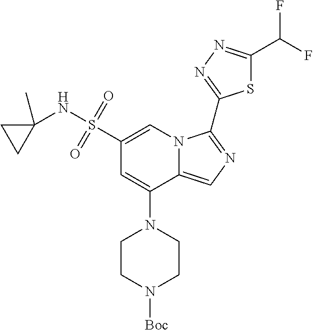 [SMILES: CC1(NS(=O)(=O)c2cc(N3CCN(C(=O)OC(C)(C)C)CC3)c3cnc(-c4nnc(C(F)F)s4)n3c2)CC1] | tert-butyl 4-(3-(5-(difluoromethyl)-1,3,4- thiadiazol-2-yl)-6-(N-(1- methylcyclopropyl)sulfamoyl)imidazo[1,5- a]pyridin-8-yl)piperazine-1-carboxylate |
| 31 | 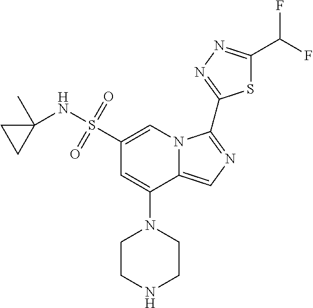 [SMILES: CC1(NS(=O)(=O)c2cc(N3CCNCC3)c3cnc(-c4nnc(C(F)F)s4)n3c2)CC1] | 3-(5-(difluoromethyl)-1,3,4-thiadiazol-2-yl)- N-(1-methylcyclopropyl)-8-(piperazin-1- yl)imidazo[1,5-a]pyridine-6-sulfonamide |
| 32 | 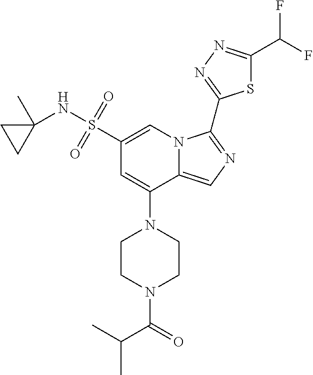 [SMILES: CC(C)C(=O)N1CCN(c2cc(S(=O)(=O)NC3(C)CC3)cn3c(-c4nnc(C(F)F)s4)ncc23)CC1] | 3-(5-(difluoromethyl)-1,3,4-thiadiazol-2-yl)-8-(4- isobutyrylpiperazin-1-yl)-N-(1- methylcyclopropyl)imidazo[1,5-a]pyridine-6- sulfonamide |
| 33 | 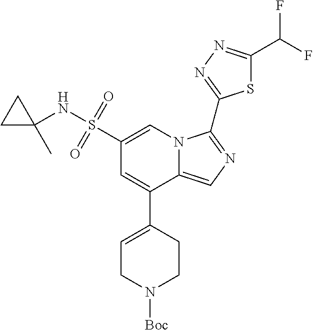 [SMILES: CC1(NS(=O)(=O)c2cc(C3=CCN(C(=O)OC(C)(C)C)CC3)c3cnc(-c4nnc(C(F)F)s4)n3c2)CC1] | tert-butyl 4-(3-(5-(difluoromethyl)-1,3,4- thiadiazol-2-yl)-6-(N-(1- methylcyclopropyl)sulfamoyl)imidazo[1,5- a]pyridin-8-yl)-3,6-dihydropyridine-1(2H)- carboxylate |
| 34 | 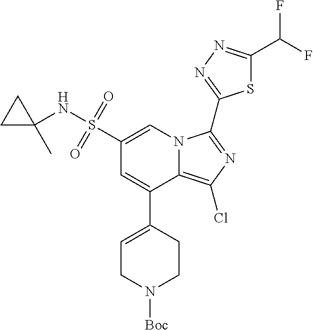 [SMILES: CC1(NS(=O)(=O)c2cc(C3=CCN(C(=O)OC(C)(C)C)CC3)c3c(Cl)nc(-c4nnc(C(F)F)s4)n3c2)CC1] | tert-butyl 4-(1-chloro-3-(5-(difluoromethyl)- 1,3,4-thiadiazol-2-yl)-6-(N-(1- methylcyclopropyl)sulfamoyl)imidazo[1,5- a]pyridin-8-yl)-3,6-dihydropyridine-1(2H)- carboxylate |
| 35 | 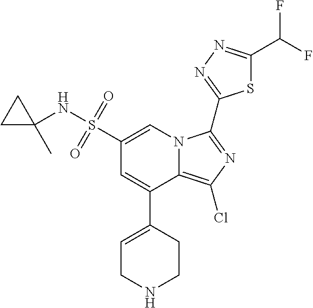 [SMILES: CC1(NS(=O)(=O)c2cc(C3=CCNCC3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(1,2,3,6- tetrahydropyridin-4-yl)imidazo[1,5-a]pyridine-6- sulfonamide |
| 36 | 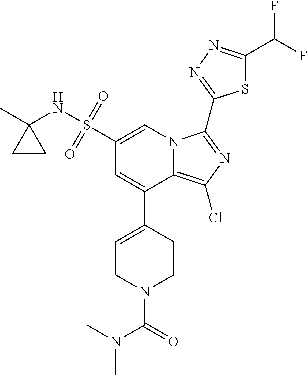 [SMILES: CN(C)C(=O)N1CC=C(c2cc(S(=O)(=O)NC3(C)CC3)cn3c(-c4nnc(C(F)F)s4)nc(Cl)c23)CC1] | 4-(1-chloro-3-(5-(difluoromethyl)-1,3,4-thiadiazol- 2-yl)-6-(N-(1- methylcyclopropyl)sulfamoyl)imidazo[1,5- a]pyridin-8-yl)-N,N-dimethyl-3,6-dihydropyridine- 1(2H)-carboxamide |
| 37 | 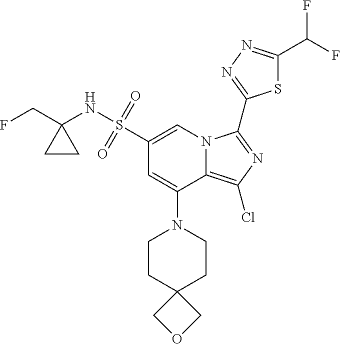 [SMILES: O=S(=O)(NC1(CF)CC1)c1cc(N2CCC3(CC2)COC3)c2c(Cl)nc(-c3nnc(C(F)F)s3)n2c1] | 1-chloro-3-(5-(difluoromethyl)-1,3,4-thiadiazol-2- yl)-N-(1-(fluoromethyl)cyclopropyl)-8-(2-oxa-7- azaspiro[3.5]nonan-7-yl)imidazo[1,5-a]pyridine- 6-sulfonamide |
| 38 | 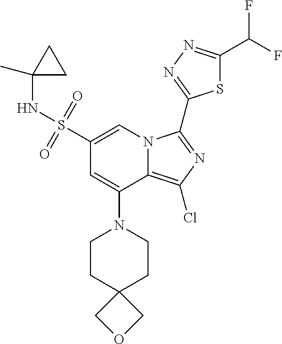 [SMILES: CC1(NS(=O)(=O)c2cc(N3CCC4(CC3)COC4)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2-oxa-7- azaspiro[3.5]nonan-7-yl)imidazo[1,5-a]pyridine- 6-sulfonamide |
| 39 | 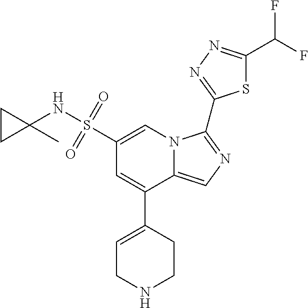 [SMILES: CC1(NS(=O)(=O)c2cc(C3=CCNCC3)c3cnc(-c4nnc(C(F)F)s4)n3c2)CC1] | 3-(5-(difluoromethyl)-1,3,4-thiadiazol-2-yl)- N-(1-methylcyclopropyl)-8-(1,2,3,6- tetrahydropyridin-4-yl)imidazo[1,5-a]pyridine-6- sulfonamide |
| 40 | 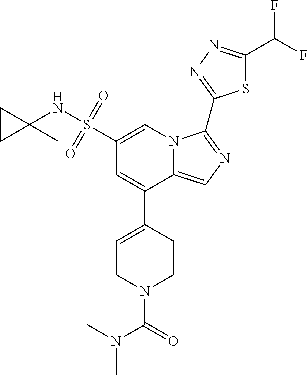 [SMILES: CN(C)C(=O)N1CC=C(c2cc(S(=O)(=O)NC3(C)CC3)cn3c(-c4nnc(C(F)F)s4)ncc23)CC1] | 4-(3-(5-(difluoromethyl)-1,3,4-thiadiazol-2-yl)-6- (N-(1-methylcyclopropyl)sulfamoyl)imidazo[1,5- a]pyridin-8-yl)-N,N-dimethyl-3,6-dihydropyridine- 1(2H)-carboxamide |
| 41a | 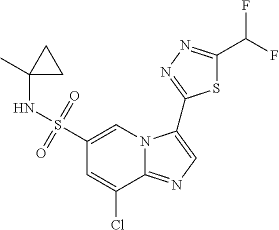 [SMILES: CC1(NS(=O)(=O)c2cc(Cl)c3ncc(-c4nnc(C(F)F)s4)n3c2)CC1] | 8-chloro-3-(5-(difluoromethyl)-1,3,4-thiadiazol-2- yl)-N-(1-methylcyclopropyl)imidazo[1,2- a]pyridine-6-sulfonamide |
| 41 | 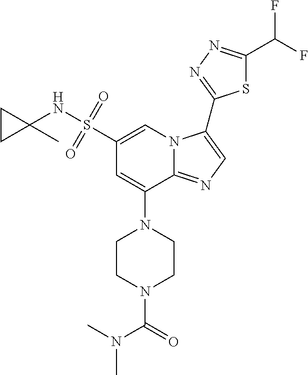 [SMILES: CN(C)C(=O)N1CCN(c2cc(S(=O)(=O)NC3(C)CC3)cn3c(-c4nnc(C(F)F)s4)cnc23)CC1] | 4-(3-(5-(difluoromethyl)-1,3,4-thiadiazol-2-yl)-6- (N-(1-methylcyclopropyl)sulfamoyl)imidazo[1,2- a]pyridin-8-yl)-N,N-dimethylpiperazine-1- carboxamide |
| 42 | 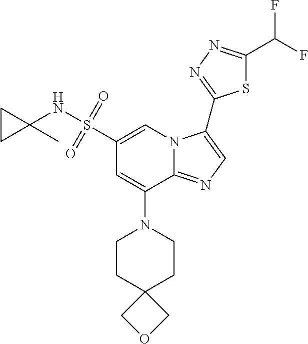 [SMILES: CC1(NS(=O)(=O)c2cc(N3CCC4(CC3)COC4)c3ncc(-c4nnc(C(F)F)s4)n3c2)CC1] | 3-(5-(difluoromethyl)-1,3,4-thiadiazol-2-yl)-N-(1- methylcyclopropyl)-8-(2-oxa-7- azaspiro[3.5]nonan-7-yl)imidazo[1,2-a]pyridine- 6-sulfonamide |
| 43 | 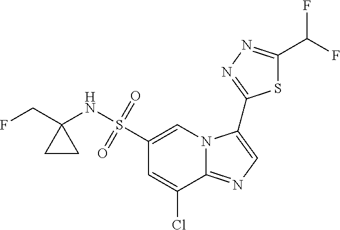 [SMILES: O=S(=O)(NC1(CF)CC1)c1cc(Cl)c2ncc(-c3nnc(C(F)F)s3)n2c1] | 8-chloro-3-(5-(difluoromethyl)-1,3,4-thiadiazol-2- yl)-N-(1-(fluoromethyl)cyclopropyl)imidazo[1,2- a]pyridine-6-sulfonamide |
| 44 | 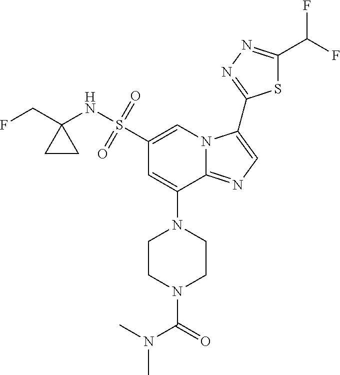 [SMILES: CN(C)C(=O)N1CCN(c2cc(S(=O)(=O)NC3(CF)CC3)cn3c(-c4nnc(C(F)F)s4)cnc23)CC1] | 4-(3-(5-(difluoromethyl)-1,3,4-thiadiazol-2-yl)-6- (N-(1- (fluoromethyl)cyclopropyl)sulfamoyl)imidazo[1,2- a]pyridin-8-yl)-N,N-dimethylpiperazine-1- carboxamide |
| 45 | 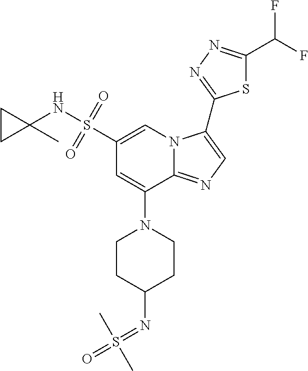 [SMILES: CC1(NS(=O)(=O)c2cc(N3CCC(N=S(C)(C)=O)CC3)c3ncc(-c4nnc(C(F)F)s4)n3c2)CC1] | 3-(5-(difluoromethyl)-1,3,4-thiadiazol-2-yl)-8-(4- ((dimethyl(oxo)-I6- sulfaneylidene)amino)piperidin-1-yl)-N-(1- methylcyclopropyl)imidazo[1,2-a]pyridine-6- sulfonamide |
| 46 | 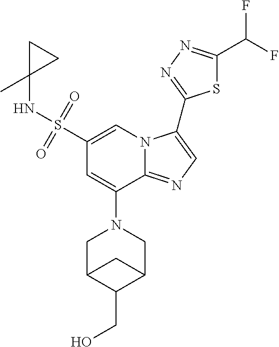 [SMILES: CC1(NS(=O)(=O)c2cc(N3CC4CC(C3)C4CO)c3ncc(-c4nnc(C(F)F)s4)n3c2)CC1] | 3-(5-(difluoromethyl)-1,3,4-thiadiazol-2-yl)-8-(6- (hydroxymethyl)-3-azabicyclo[3.1.1]heptan-3-yl)- N-(1-methylcyclopropyl)imidazo[1,2-a]pyridine-6- sulfonamide |
| 47 | 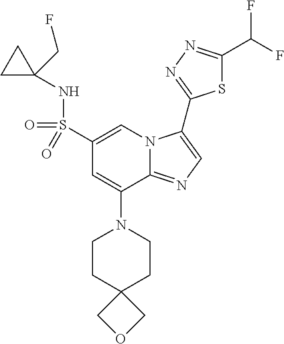 [SMILES: O=S(=O)(NC1(CF)CC1)c1cc(N2CCC3(CC2)COC3)c2ncc(-c3nnc(C(F)F)s3)n2c1] | 33-(5-(difluoromethyl)-1,3,4-thiadiazol-2-yl)-N-(1- (fluoromethyl)cyclopropyl)-8-(2-oxa-7- azaspiro[3.5]nonan-7-yl)imidazo[1,2-a]pyridine- 6-sulfonamide |
| 48 | 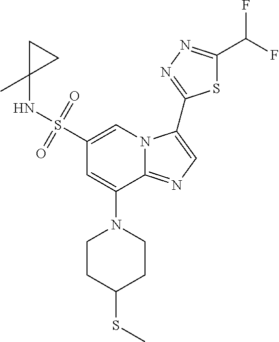 [SMILES: CSC1CCN(c2cc(S(=O)(=O)NC3(C)CC3)cn3c(-c4nnc(C(F)F)s4)cnc23)CC1] | 3-(5-(difluoromethyl)-1,3,4-thiadiazol-2-yl)-N-(1- methylcyclopropyl)-8-(4-(methylthio)piperidin-1- yl)imidazo[1,2-a]pyridine-6-sulfonamide |
| 49 | 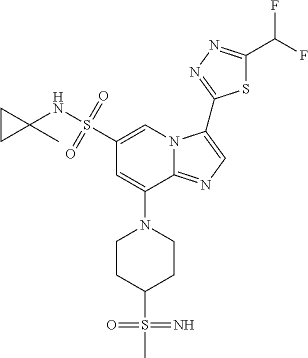 [SMILES: CC1(NS(=O)(=O)c2cc(N3CCC(S(C)(=N)=O)CC3)c3ncc(-c4nnc(C(F)F)s4)n3c2)CC1] | 3-(5-(difluoromethyl)-1,3,4-thiadiazol-2-yl)-N-(1- methylcyclopropyl)-8-(4-(S- methylsulfonimidoyl)piperidin-1-yl)imidazo[1,2- a]pyridine-6-sulfonamide |
| 50 | 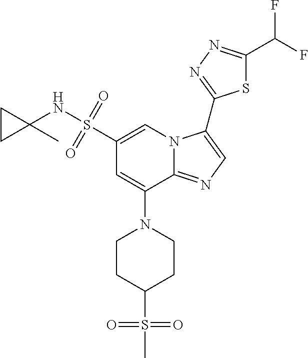 [SMILES: CC1(NS(=O)(=O)c2cc(N3CCC(S(C)(=O)=O)CC3)c3ncc(-c4nnc(C(F)F)s4)n3c2)CC1] | 3-(5-(difluoromethyl)-1,3,4-thiadiazol-2-yl)-N-(1- methylcyclopropyl)-8-(4- (methylsulfonyl)piperidin-1-yl)imidazo[1,2- a]pyridine-6-sulfonamide |
| 51 | 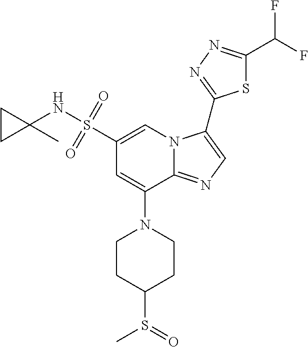 [SMILES: CS(=O)C1CCN(c2cc(S(=O)(=O)NC3(C)CC3)cn3c(-c4nnc(C(F)F)s4)cnc23)CC1] | 3-(5-(difluoromethyl)-1,3,4-thiadiazol-2-yl)-N-(1- methylcyclopropyl)-8-(4-(methylsulfinyl)piperidin- 1-yl)imidazo[1,2-a]pyridine-6-sulfonamide |
| 52 | 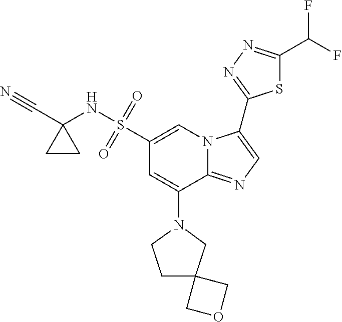 [SMILES: N#CC1(NS(=O)(=O)c2cc(N3CCC4(COC4)C3)c3ncc(-c4nnc(C(F)F)s4)n3c2)CC1] | N-(1-cyanocyclopropyl)-3-(5-(difluoromethyl)- 1,3,4-thiadiazol-2-yl)-8-(2-oxa-6- azaspiro[3.4]octan-6-yl)imidazo[1,2-a]pyridine-6- sulfonamide |
| 53 | 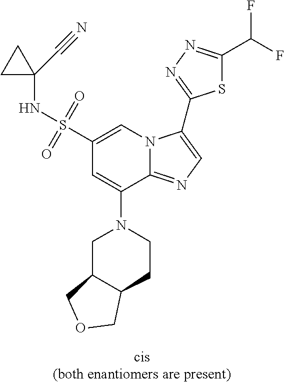 [SMILES: N#CC1(NS(=O)(=O)c2cc(N3CC[C@H]4COC[C@H]4C3)c3ncc(-c4nnc(C(F)F)s4)n3c2)CC1] | N-(1-cyanocyclopropyl)-3-(5-(difluoromethyl)- 1,3,4-thiadiazol-2-yl)-8-((cis)-hexahydrofuro[3,4- c]pyridin-5(3H)-yl)imidazo[1,2-a]pyridine-6- sulfonamide |
| 54 | 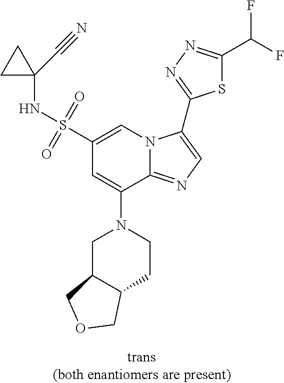 [SMILES: N#CC1(NS(=O)(=O)c2cc(N3CC[C@@H]4COC[C@H]4C3)c3ncc(-c4nnc(C(F)F)s4)n3c2)CC1] | N-(1-cyanocyclopropyl)-3-(5-(difluoromethyl)- 1,3,4-thiadiazol-2-yl)-8-((trans)- hexahydrofuro[3,4-c]pyridin-5(3H)- yl)imidazo[1,2-a]pyridine-6-sulfonamide |
| 55 | 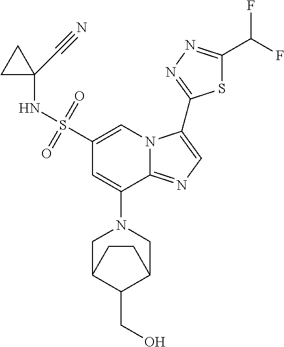 [SMILES: N#CC1(NS(=O)(=O)c2cc(N3CC4CCC(C3)C4CO)c3ncc(-c4nnc(C(F)F)s4)n3c2)CC1] | N-(1-cyanocyclopropyl)-3-(5-(difluoromethyl)- 1,3,4-thiadiazol-2-yl)-8-(8-(hydroxymethyl)-3- azabicyclo[3.2.1]octan-3-yl)imidazo[1,2- a]pyridine-6-sulfonamide |
| 56 | 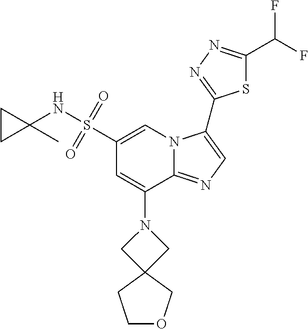 [SMILES: CC1(NS(=O)(=O)c2cc(N3CC4(CCOC4)C3)c3ncc(-c4nnc(C(F)F)s4)n3c2)CC1] | 3-(5-(difluoromethyl)-1,3,4-thiadiazol-2-yl)-N-(1- methylcyclopropyl)-8-(6-oxa-2- azaspiro[3.4]octan-2-yl)imidazo[1,2-a]pyridine-6- sulfonamide |
| 57 | 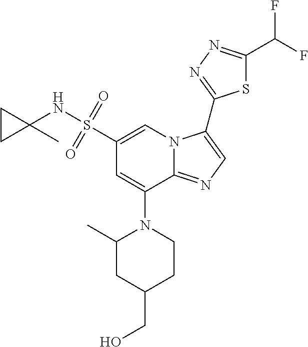 [SMILES: CC1CC(CO)CCN1c1cc(S(=O)(=O)NC2(C)CC2)cn2c(-c3nnc(C(F)F)s3)cnc12] | 3-(5-(difluoromethyl)-1,3,4-thiadiazol-2-yl)-8-(4- (hydroxymethyl)-2-methylpiperidin-1-yl)-N-(1- methylcyclopropyl)imidazo[1,2-a]pyridine-6- sulfonamide |
| 58 | 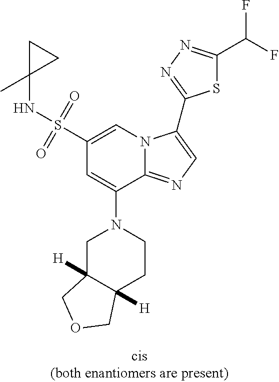 [SMILES: CC1(NS(=O)(=O)c2cc(N3CC[C@@H]4COC[C@@H]4C3)c3ncc(-c4nnc(C(F)F)s4)n3c2)CC1] | 3-(5-(difluoromethyl)-1,3,4-thiadiazol-2-yl)-8- ((cis)-hexahydrofuro[3,4-c]pyridin-5(3H)-yl)-N-(1- methylcyclopropyl)imidazo[1,2-a]pyridine-6- sulfonamide |
| 59 | 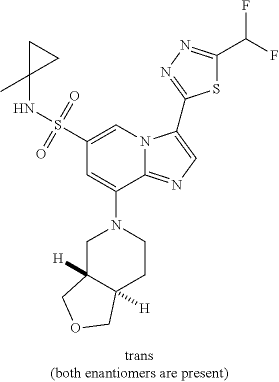 [SMILES: CC1(NS(=O)(=O)c2cc(N3CC[C@H]4COC[C@@H]4C3)c3ncc(-c4nnc(C(F)F)s4)n3c2)CC1] | 3-(5-(difluoromethyl)-1,3,4-thiadiazol-2-yl)-8- ((trans)-hexahydrofuro[3,4-c]pyridin-5(3H)-yl)-N- (1-methylcyclopropyl)imidazo[1,2-a]pyridine-6- sulfonamide |
| 60 | 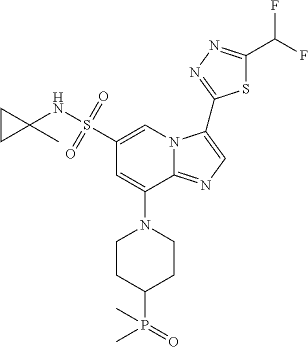 [SMILES: CC1(NS(=O)(=O)c2cc(N3CCC(P(C)(C)=O)CC3)c3ncc(-c4nnc(C(F)F)s4)n3c2)CC1] | 3-(5-(difluoromethyl)-1,3,4-thiadiazol-2-yl)-8-(4- (dimethylphosphoryl)piperidin-1-yl)-N-(1- methylcyclopropyl)imidazo[1,2-a]pyridine-6- sulfonamide |
| 61 | 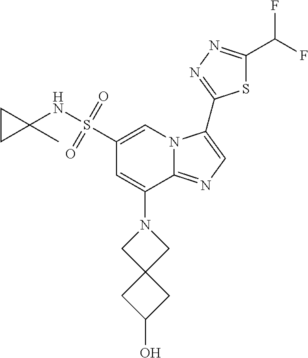 [SMILES: CC1(NS(=O)(=O)c2cc(N3CC4(CC(O)C4)C3)c3ncc(-c4nnc(C(F)F)s4)n3c2)CC1] | 3-(5-(difluoromethyl)-1,3,4-thiadiazol-2-yl)-8-(6- hydroxy-2-azaspiro[3.3]heptan-2-yl)-N-(1- methylcyclopropyl)imidazo[1,2-a]pyridine-6- sulfonamide |
| 62 | 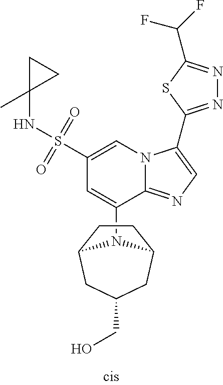 [SMILES: CC1(NS(=O)(=O)c2cc(N3[C@@H]4CC[C@H]3C[C@H](CO)C4)c3ncc(-c4nnc(C(F)F)s4)n3c2)CC1] | 3-(5-(difluoromethyl)-1,3,4-thiadiazol-2-yl)-8- ((1R,3s,5S)-3-(hydroxymethyl)-8- azabicyclo[3.2.1]octan-8-yl)-N-(1- methylcyclopropyl)imidazo[1,2-a]pyridine-6- sulfonamide |
| 63 | 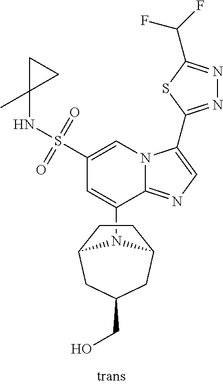 [SMILES: CC1(NS(=O)(=O)c2cc(N3[C@@H]4CC[C@H]3C[C@@H](CO)C4)c3ncc(-c4nnc(C(F)F)s4)n3c2)CC1] | 3-(5-(difluoromethyl)-1,3,4-thiadiazol-2-yl)-8- ((1R,3r,5S)-3-(hydroxymethyl)-8- azabicyclo[3.2.1]octan-8-yl)-N-(1- methylcyclopropyl)imidazo[1,2-a]pyridine-6- sulfonamide |
| 64 | 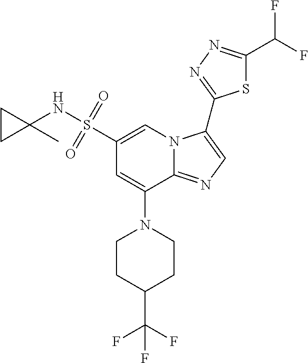 [SMILES: CC1(NS(=O)(=O)c2cc(N3CCC(C(F)(F)F)CC3)c3ncc(-c4nnc(C(F)F)s4)n3c2)CC1] | 3-(5-(difluoromethyl)-1,3,4-thiadiazol-2-yl)-N-(1- methylcyclopropyl)-8-(4- (trifluoromethyl)piperidin-1-yl)imidazo[1,2- a]pyridine-6-sulfonamide |
| 65 | 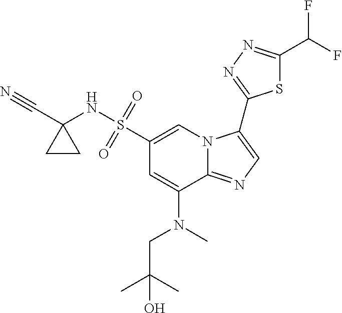 [SMILES: CN(CC(C)(C)O)c1cc(S(=O)(=O)NC2(C#N)CC2)cn2c(-c3nnc(C(F)F)s3)cnc12] | N-(1-cyanocyclopropyl)-3-(5-(difluoromethyl)- 1,3,4-thiadiazol-2-yl)-8-((2-hydroxy-2- methylpropyl)(methyl)amino)imidazo[1,2- a]pyridine-6-sulfonamide |
| 66 | 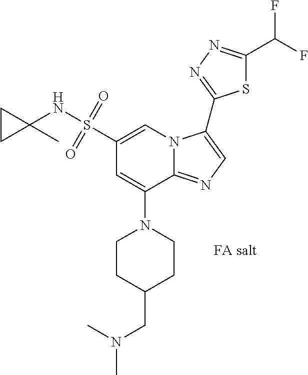 | 3-(5-(difluoromethyl)-1,3,4-thiadiazol-2-yl)-8-(4- ((dimethylamino)methyl)piperidin-1-yl)-N-(1- methylcyclopropyl)imidazo[1,2-a]pyridine-6- sulfonamide formate |
| 67 | 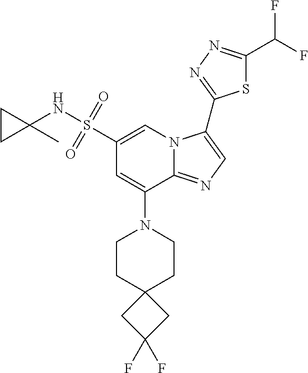 [SMILES: CC1(NS(=O)(=O)c2cc(N3CCC4(CC3)CC(F)(F)C4)c3ncc(-c4nnc(C(F)F)s4)n3c2)CC1] | 8-(2,2-difluoro-7-azaspiro[3.5]nonan-7-yl)-3-(5- (difluoromethyl)-1,3,4-thiadiazol-2-yl)-N-(1- methylcyclopropyl)imidazo[1,2-a]pyridine-6- sulfonamide |
| 68 | 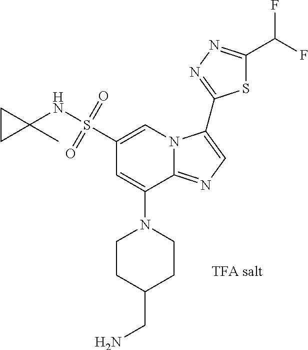 | 8-(4-(aminomethyl)piperidin-1-yl)-3-(5- (difluoromethyl)-1,3,4-thiadiazol-2-yl)-N-(1- methylcyclopropyl)imidazo[1,2-a]pyridine-6- sulfonamide 2,2,2-trifluoroacetate |
| 69 | 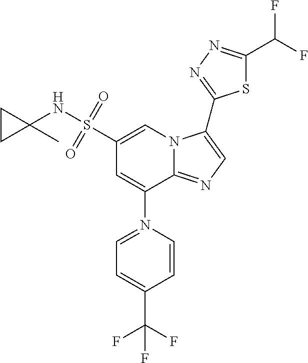 | 3-(5-(difluoromethyl)-1,3,4-thiadiazol-2-yl)-N-(1- methylcyclopropyl)-8-(4- (trifluoromethyl)phenyl)imidazo[1,2-a]pyridine-6- sulfonamide |
| 70a | 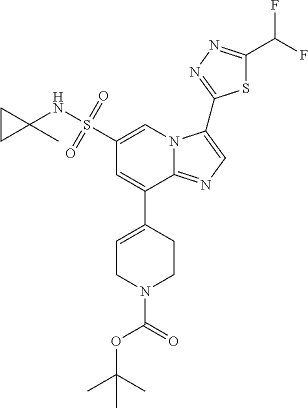 [SMILES: CC1(NS(=O)(=O)c2cc(C3=CCN(C(=O)OC(C)(C)C)CC3)c3ncc(-c4nnc(C(F)F)s4)n3c2)CC1] | tert-butyl 4-(3-(5-(difluoromethyl)-1,3,4-thiadiazol- 2-yl)-6-(N-(1- methylcyclopropyl)sulfamoyl)imidazo[1,2- a]pyridin-8-yl)-3,6-dihydropyridine-1(2H)- carboxylate |
| 70 | 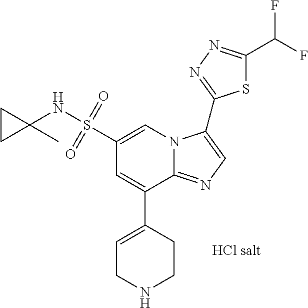 | 3-(5-(difluoromethyl)-1,3,4-thiadiazol-2-yl)-N-(1- methylcyclopropyl)-8-(1,2,3,6-tetrahydropyridin- 4-yl)imidazo[1,2-a]pyridine-6-sulfonamide hydrogen chloride |
| 71 | 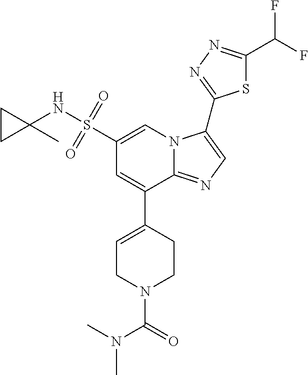 [SMILES: CN(C)C(=O)N1CC=C(c2cc(S(=O)(=O)NC3(C)CC3)cn3c(-c4nnc(C(F)F)s4)cnc23)CC1] | 4-(3-(5-(difluoromethyl)-1,3,4-thiadiazol-2-yl)-6- (N-(1-methylcyclopropyl)sulfamoyl)imidazo[1,2- a]pyridin-8-yl)-N,N-dimethyl-3,6-dihydropyridine- 1(2H)-carboxamide |
| 72 | 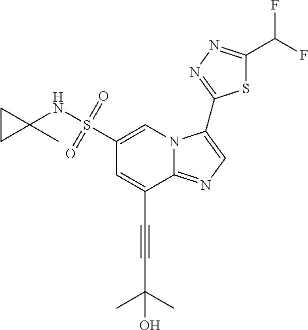 [SMILES: CC(C)(O)C#Cc1cc(S(=O)(=O)NC2(C)CC2)cn2c(-c3nnc(C(F)F)s3)cnc12] | 3-(5-(difluoromethyl)-1,3,4-thiadiazol-2-yl)-8-(3- hydroxy-3-methylbut-1-yn-1-yl)-N-(1- methylcyclopropyl)imidazo[1,2-a]pyridine-6- sulfonamide |
| 73 | 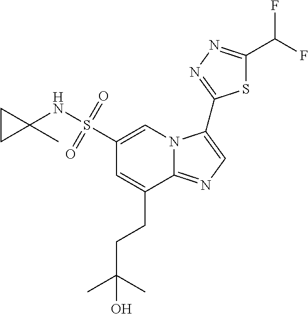 [SMILES: CC(C)(O)CCc1cc(S(=O)(=O)NC2(C)CC2)cn2c(-c3nnc(C(F)F)s3)cnc12] | 3-(5-(difluoromethyl)-1,3,4-thiadiazol-2-yl)-8-(3- hydroxy-3-methylbutyl)-N-(1- methylcyclopropyl)imidazo[1,2-a]pyridine-6- sulfonamide |
| 74 | 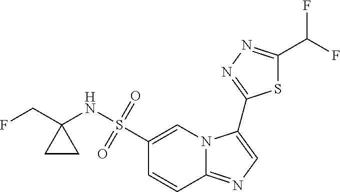 [SMILES: O=S(=O)(NC1(CF)CC1)c1ccc2ncc(-c3nnc(C(F)F)s3)n2c1] | 3-(5-(difluoromethyl)-1,3,4-thiadiazol-2-yl)-N-(1- (fluoromethyl)cyclopropyl)imidazo[1,2-a]pyridine- 6-sulfonamide |
| 75 | 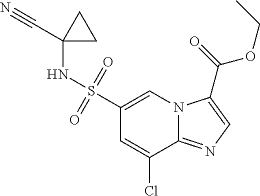 [SMILES: CCOC(=O)c1cnc2c(Cl)cc(S(=O)(=O)NC3(C#N)CC3)cn12] | Ethyl 8-chloro-6-(N-(1- cyanocyclopropyl)sulfamoyl)imidazo[1,2- a]pyridine-3-carboxylate |
| 76 | 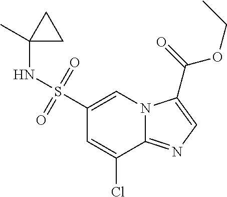 [SMILES: CCOC(=O)c1cnc2c(Cl)cc(S(=O)(=O)NC3(C)CC3)cn12] | Ethyl 8-chloro-6-(N-(1- methylcyclopropyl)sulfamoyl)imidazo[1,2- a]pyridine-3-carboxylate. |
| 77 | 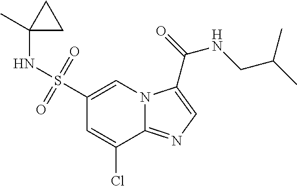 [SMILES: CC(C)CNC(=O)c1cnc2c(Cl)cc(S(=O)(=O)NC3(C)CC3)cn12] | 8-chloro-N-isobutyl-6-(N-(1- methylcyclopropyl)sulfamoyl)imidazo[1,2- a]pyridine-3-carboxamide |
| 78 | 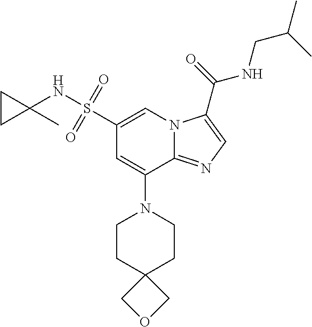 [SMILES: CC(C)CNC(=O)c1cnc2c(N3CCC4(CC3)COC4)cc(S(=O)(=O)NC3(C)CC3)cn12] | N-isobutyl-6-(N-(1-methylcyclopropyl)sulfamoyl)- 8-(2-oxa-7-azaspiro[3.5]nonan-7-yl)imidazo[1,2- a]pyridine-3-carboxamide |
| 79 | 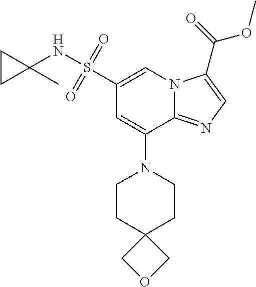 [SMILES: COC(=O)c1cnc2c(N3CCC4(CC3)COC4)cc(S(=O)(=O)NC3(C)CC3)cn12] | methyl 6-(N-(1-methylcyclopropyl)sulfamoyl)-8- (2-oxa-7-azaspiro[3.5]nonan-7-yl)imidazo[1,2- a]pyridine-3-carboxylate |
| 80 | 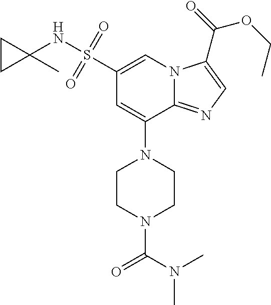 [SMILES: CCOC(=O)c1cnc2c(N3CCN(C(=O)N(C)C)CC3)cc(S(=O)(=O)NC3(C)CC3)cn12] | Ethyl 8-(4-(dimethylcarbamoyl)piperazin-1-yl)-6- (N-(1-methylcyclopropyl)sulfamoyl)imidazo[1,2- a]pyridine-3-carboxylate |
| 81 | 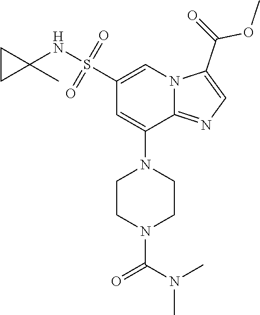 [SMILES: COC(=O)c1cnc2c(N3CCN(C(=O)N(C)C)CC3)cc(S(=O)(=O)NC3(C)CC3)cn12] | Methyl 8-(4-(dimethylcarbamoyl)piperazin-1-yl)-6- (N-(1-methylcyclopropyl)sulfamoyl)imidazo[1,2- a]pyridine-3-carboxylate |
| 82 | 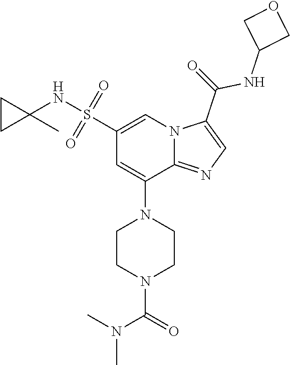 [SMILES: CN(C)C(=O)N1CCN(c2cc(S(=O)(=O)NC3(C)CC3)cn3c(C(=O)NC4COC4)cnc23)CC1] | 8-(4-(dimethylcarbamoyl)piperazin-1-yl)-6-(N-(1- methylcyclopropyl)sulfamoyl)-N-(oxetan-3- yl)imidazo[1,2-a]pyridine-3-carboxamide |
| 83 | 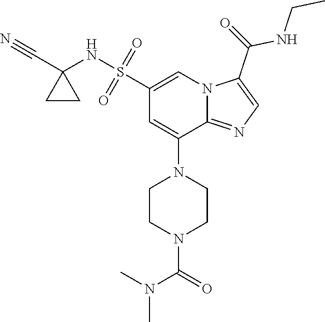 [SMILES: CCNC(=O)c1cnc2c(N3CCN(C(=O)N(C)C)CC3)cc(S(=O)(=O)NC3(C#N)CC3)cn12] | 6-(N-(1-cyanocyclopropyl)sulfamoyl)-8-(4- (dimethylcarbamoyl)piperazin-1-yl)-N- ethylimidazo[1,2-a]pyridine-3-carboxamide |
| 84 | 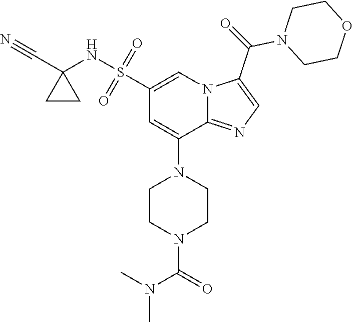 [SMILES: CN(C)C(=O)N1CCN(c2cc(S(=O)(=O)NC3(C#N)CC3)cn3c(C(=O)N4CCOCC4)cnc23)CC1] | 4-(6-(N-(1-cyanocyclopropyl)sulfamoyl)-3- (morpholine-4-carbonyl)imidazo[1,2-a]pyridin-8- yl)-N,N-dimethylpiperazine-1-carboxamide |
| 85 | 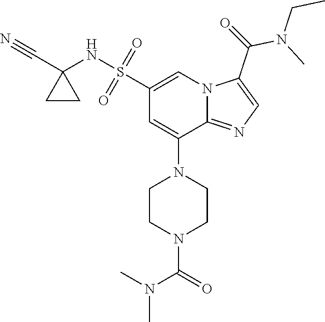 [SMILES: CCN(C)C(=O)c1cnc2c(N3CCN(C(=O)N(C)C)CC3)cc(S(=O)(=O)NC3(C#N)CC3)cn12] | 6-(N-(1-cyanocyclopropyl)sulfamoyl)-8-(4- (dimethylcarbamoyl)piperazin-1-yl)-N-ethyl-N- methylimidazo[1,2-a]pyridine-3-carboxamide |
| 86 | 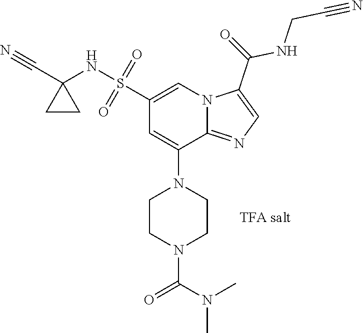 [SMILES: CN(C)C(=O)N1CCN(c2cc(S(=O)(=O)NC3(C#N)CC3)cn3c(C(=O)NCC#N)cnc23)CC1] | 6-(N-(1-cyanocyclopropyl)sulfamoyl)-N- (cyanomethyl)-8-(4- (dimethylcarbamoyl)piperazin-1-yl)imidazo[1,2- a]pyridine-3-carboxamide 2,2,2-trifluoroacetate |
| 87 | 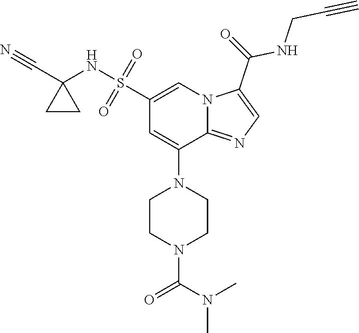 [SMILES: C#CCNC(=O)c1cnc2c(N3CCN(C(=O)N(C)C)CC3)cc(S(=O)(=O)NC3(C#N)CC3)cn12] | 6-(N-(1-cyanocyclopropyl)sulfamoyl)-8-(4- (dimethylcarbamoyl)piperazin-1-yl)-N-(prop-2-yn- 1-yl)imidazo[1,2-a]pyridine-3-carboxamide |
| 88 | 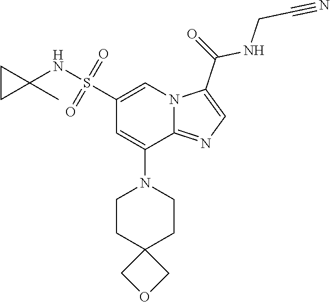 [SMILES: CC1(NS(=O)(=O)c2cc(N3CCC4(CC3)COC4)c3ncc(C(=O)NCC#N)n3c2)CC1] | N-(cyanomethyl)-6-(N-(1- methylcyclopropyl)sulfamoyl)-8-(2-oxa-7- azaspiro[3.5]nonan-7-yl)imidazo[1,2-a]pyridine- 3-carboxamide |
| 89 | 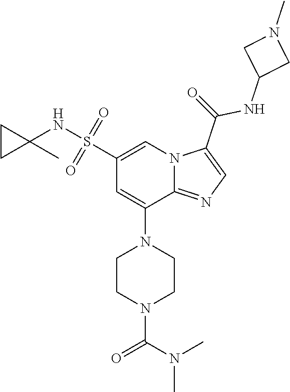 [SMILES: CN1CC(NC(=O)c2cnc3c(N4CCN(C(=O)N(C)C)CC4)cc(S(=O)(=O)NC4(C)CC4)cn23)C1] | 8-(4-(dimethylcarbamoyl)piperazin-1-yl)-N-(1- methylazetidin-3-yl)-6-(N-(1- methylcyclopropyl)sulfamoyl)imidazo[1,2- a]pyridine-3-carboxamide |
| 90 | 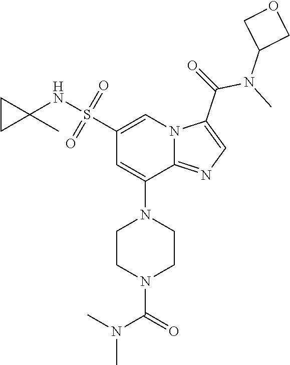 [SMILES: CN(C)C(=O)N1CCN(c2cc(S(=O)(=O)NC3(C)CC3)cn3c(C(=O)N(C)C4COC4)cnc23)CC1] | 8-(4-(dimethylcarbamoyl)piperazin-1-yl)-N- methyl-6-(N-(1-methylcyclopropyl)sulfamoyl)-N- (oxetan-3-yl)imidazo[1,2-a]pyridine-3- carboxamide |
| 91 | 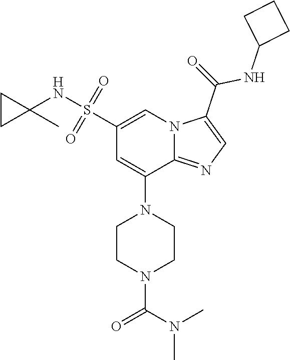 [SMILES: CN(C)C(=O)N1CCN(c2cc(S(=O)(=O)NC3(C)CC3)cn3c(C(=O)NC4CCC4)cnc23)CC1] | N-cyclobutyl-8-(4-(dimethylcarbamoyl)piperazin- 1-yl)-6-(N-(1- methylcyclopropyl)sulfamoyl)imidazo[1,2- a]pyridine-3-carboxamide |
| 92 | 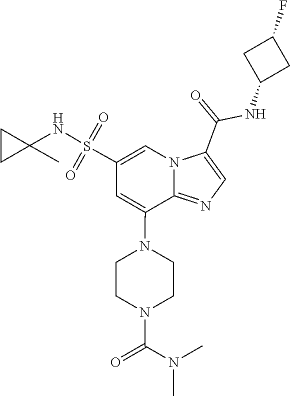 [SMILES: CN(C)C(=O)N1CCN(c2cc(S(=O)(=O)NC3(C)CC3)cn3c(C(=O)N[C@H]4C[C@@H](F)C4)cnc23)CC1] | 8-(4-(dimethylcarbamoyl)piperazin-1-yl)-N- ((1s,3s)-3-fluorocyclobutyl)-6-(N-(1- methylcyclopropyl)sulfamoyl)imidazo[1,2- a]pyridine-3-carboxamide |
| 93 | 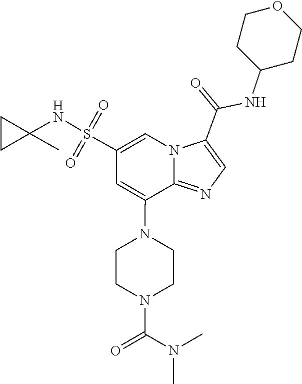 [SMILES: CN(C)C(=O)N1CCN(c2cc(S(=O)(=O)NC3(C)CC3)cn3c(C(=O)NC4CCOCC4)cnc23)CC1] | 8-(4-(dimethylcarbamoyl)piperazin-1-yl)-6-(N-(1- methylcyclopropyl)sulfamoyl)-N-(tetrahydro-2H- pyran-4-yl)imidazo[1,2-a]pyridine-3-carboxamide |
| 94 | 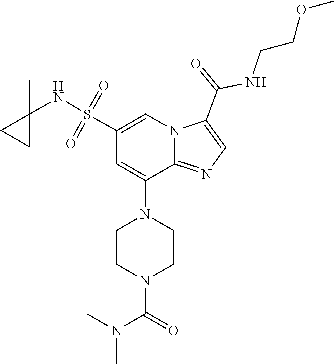 [SMILES: COCCNC(=O)c1cnc2c(N3CCN(C(=O)N(C)C)CC3)cc(S(=O)(=O)NC3(C)CC3)cn12] | 8-(4-(dimethylcarbamoyl)piperazin-1-yl)-N-(2- methoxyethyl)-6-(N-(1- methylcyclopropyl)sulfamoyl)imidazo[1,2- a]pyridine-3-carboxamide |
| 95 | 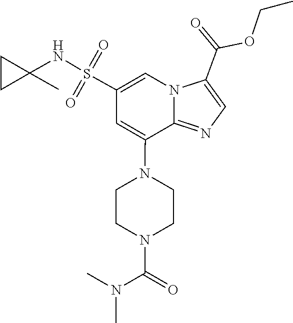 [SMILES: CCOC(=O)c1cnc2c(N3CCN(C(=O)N(C)C)CC3)cc(S(=O)(=O)NC3(C)CC3)cn12] | ethyl 8-(4-(dimethylcarbamoyl)piperazin-1-yl)-6- (N-(1-methylcyclopropyl)sulfamoyl)imidazo[1,2- a]pyridine-3-carboxylate |
| 96 | 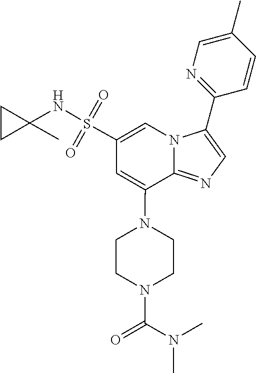 [SMILES: Cc1ccc(-c2cnc3c(N4CCN(C(=O)N(C)C)CC4)cc(S(=O)(=O)NC4(C)CC4)cn23)nc1] | N,N-dimethyl-4-(6-(N-(1- methylcyclopropyl)sulfamoyl)-3-(5-methylpyridin- 2-yl)imidazo[1,2-a]pyridin-8-yl)piperazine-1- carboxamide |
| 97 | 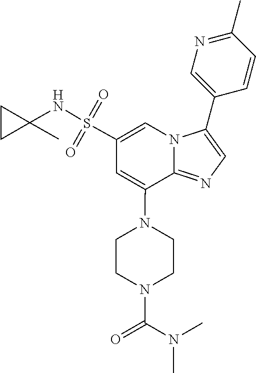 [SMILES: Cc1ccc(-c2cnc3c(N4CCN(C(=O)N(C)C)CC4)cc(S(=O)(=O)NC4(C)CC4)cn23)cn1] | N,N-dimethyl-4-(6-(N-(1- methylcyclopropyl)sulfamoyl)-3-(6-methylpyridin- 3-yl)imidazo[1,2-a]pyridin-8-yl)piperazine-1- carboxamide |
| 98 | 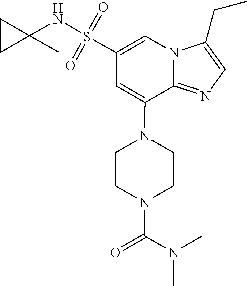 [SMILES: CCc1cnc2c(N3CCN(C(=O)N(C)C)CC3)cc(S(=O)(=O)NC3(C)CC3)cn12] | 4-(3-ethyl-6-(N-(1- methylcyclopropyl)sulfamoyl)imidazo[1,2- a]pyridin-8-yl)-N,N-dimethylpiperazine-1- carboxamide |
| 99 | 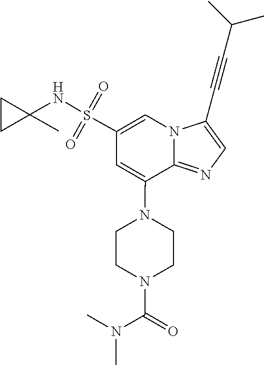 [SMILES: CC(C)C#Cc1cnc2c(N3CCN(C(=O)N(C)C)CC3)cc(S(=O)(=O)NC3(C)CC3)cn12] | N,N-dimethyl-4-(3-(3-methylbut-1-yn-1-yl)-6-(N- (1-methylcyclopropyl)sulfamoyl)imidazo[1,2- a]pyridin-8-yl)piperazine-1-carboxamide |
| 100 | 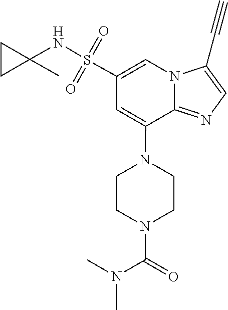 [SMILES: C#Cc1cnc2c(N3CCN(C(=O)N(C)C)CC3)cc(S(=O)(=O)NC3(C)CC3)cn12] | 4-(3-ethynyl-6-(N-(1- methylcyclopropyl)sulfamoyl)imidazo[1,2- a]pyridin-8-yl)-N,N-dimethylpiperazine-1- carboxamide |
| 101 | 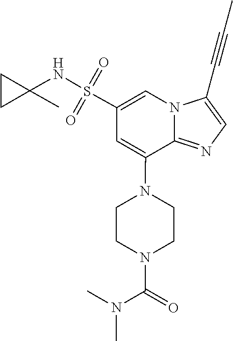 [SMILES: CC#Cc1cnc2c(N3CCN(C(=O)N(C)C)CC3)cc(S(=O)(=O)NC3(C)CC3)cn12] | N,N-dimethyl-4-(6-(N-(1- methylcyclopropyl)sulfamoyl)-3-(prop-1-yn-1- yl)imidazo[1,2-a]pyridin-8-yl)piperazine-1- carboxamide |
| 102 | 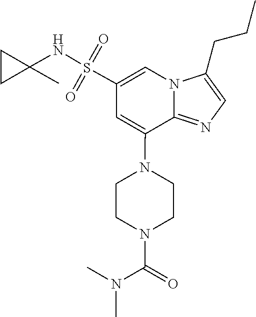 [SMILES: CCCc1cnc2c(N3CCN(C(=O)N(C)C)CC3)cc(S(=O)(=O)NC3(C)CC3)cn12] | N,N-dimethyl-4-(6-(N-(1- methylcyclopropyl)sulfamoyl)-3- propylimidazo[1,2-a]pyridin-8-yl)piperazine-1- carboxamide |
| 103 | 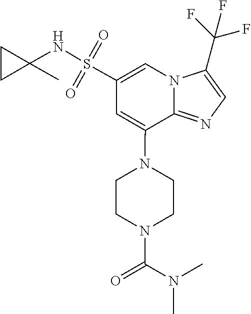 [SMILES: CN(C)C(=O)N1CCN(c2cc(S(=O)(=O)NC3(C)CC3)cn3c(C(F)(F)F)cnc23)CC1] | N,N-dimethyl-4-(6-(N-(1- methylcyclopropyl)sulfamoyl)-3- (trifluoromethyl)imidazo[1,2-a]pyridin-8- yl)piperazine-1-carboxamide |
| 105 | 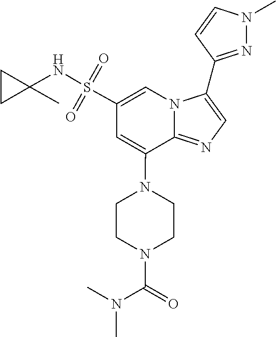 [SMILES: CN(C)C(=O)N1CCN(c2cc(S(=O)(=O)NC3(C)CC3)cn3c(-c4ccn(C)n4)cnc23)CC1] | N,N-dimethyl-4-(3-(1-methyl-1H-pyrazol-3-yl)-6- (N-(1-methylcyclopropyl)sulfamoyl)imidazo[1,2- a]pyridin-8-yl)piperazine-1-carboxamide |
| 106 | 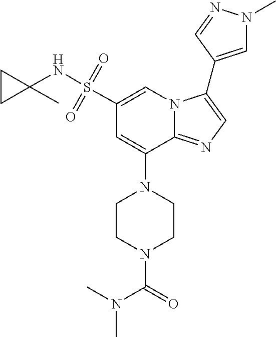 [SMILES: CN(C)C(=O)N1CCN(c2cc(S(=O)(=O)NC3(C)CC3)cn3c(-c4cnn(C)c4)cnc23)CC1] | N,N-dimethyl-4-(3-(1-methyl-1H-pyrazol-4-yl)-6- (N-(1-methylcyclopropyl)sulfamoyl)imidazo[1,2- a]pyridin-8-yl)piperazine-1-carboxamide |
| 107 | 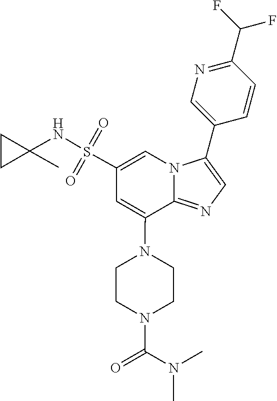 [SMILES: CN(C)C(=O)N1CCN(c2cc(S(=O)(=O)NC3(C)CC3)cn3c(-c4ccc(C(F)F)nc4)cnc23)CC1] | 4-(3-(6-(difluoromethyl)pyridin-3-yl)-6-(N-(1- methylcyclopropyl)sulfamoyl)imidazo[1,2- a]pyridin-8-yl)-N,N-dimethylpiperazine-1- carboxamide |
| 108 | 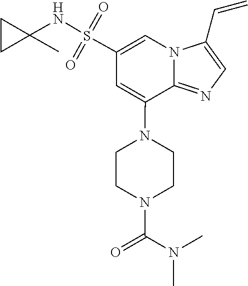 [SMILES: C=Cc1cnc2c(N3CCN(C(=O)N(C)C)CC3)cc(S(=O)(=O)NC3(C)CC3)cn12] | N,N-dimethyl-4-(6-(N-(1- methylcyclopropyl)sulfamoyl)-3-vinylimidazo[1,2- a]pyridin-8-yl)piperazine-1-carboxamide |
| 109 | 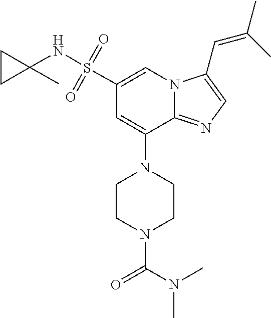 [SMILES: CC(C)=Cc1cnc2c(N3CCN(C(=O)N(C)C)CC3)cc(S(=O)(=O)NC3(C)CC3)cn12] | N,N-dimethyl-4-(6-(N-(1- methylcyclopropyl)sulfamoyl)-3-(2-methylprop-1- en-1-yl)imidazo[1,2-a]pyridin-8-yl)piperazine-1- carboxamide |
| 110 | 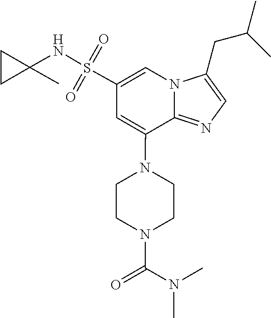 [SMILES: CC(C)Cc1cnc2c(N3CCN(C(=O)N(C)C)CC3)cc(S(=O)(=O)NC3(C)CC3)cn12] | 4-(3-isobutyl-6-(N-(1- methylcyclopropyl)sulfamoyl)imidazo[1,2- a]pyridin-8-yl)-N,N-dimethylpiperazine-1- carboxamide |
| 111 | 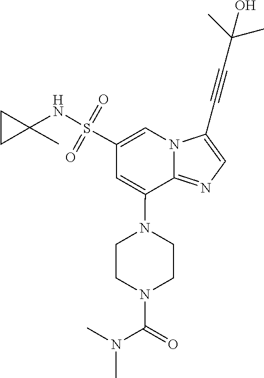 [SMILES: CN(C)C(=O)N1CCN(c2cc(S(=O)(=O)NC3(C)CC3)cn3c(C#CC(C)(C)O)cnc23)CC1] | N,N-dimethyl-4-(3-(3-methylbut-1-yn-1-yl)-6-(N- (1-methylcyclopropyl)sulfamoyl)imidazo[1,2- a]pyridin-8-yl)piperazine-1-carboxamide |
| 113 | 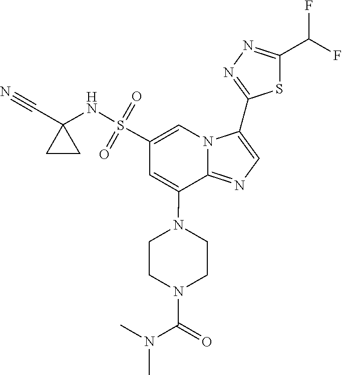 [SMILES: CN(C)C(=O)N1CCN(c2cc(S(=O)(=O)NC3(C#N)CC3)cn3c(-c4nnc(C(F)F)s4)cnc23)CC1] | 4-(6-(N-(1-cyanocyclopropyl)sulfamoyl)-3-(5- (difluoromethyl)-1,3,4-thiadiazol-2-yl)indolizin-8- yl)-N,N-dimethylpiperazine-1-carboxamide |
| 114 | 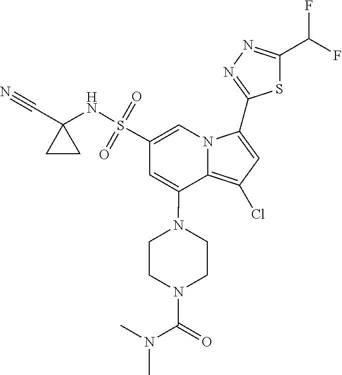 [SMILES: CN(C)C(=O)N1CCN(c2cc(S(=O)(=O)NC3(C#N)CC3)cn3c(-c4nnc(C(F)F)s4)cc(Cl)c23)CC1] | 4-(1-chloro-6-(N-(1-cyanocyclopropyl)sulfamoyl)- 3-(5-(difluoromethyl)-1,3,4-thiadiazol-2- yl)indolizin-8-yl)-N,N-dimethylpiperazine-1- carboxamide |
| 115 | 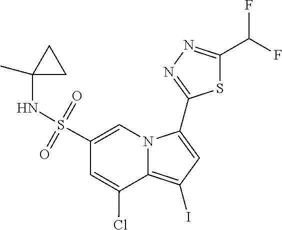 [SMILES: CC1(NS(=O)(=O)c2cc(Cl)c3c(I)cc(-c4nnc(C(F)F)s4)n3c2)CC1] | 8-chloro-3-(5-(difluoromethyl)-1,3,4-thiadiazol-2- yl)-1-iodo-N-(1-methylcyclopropyl)indolizine-6- sulfonamide |
| 116 | 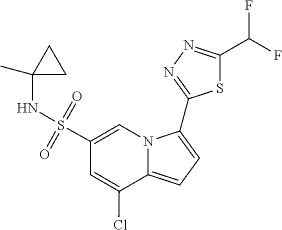 [SMILES: CC1(NS(=O)(=O)c2cc(Cl)c3ccc(-c4nnc(C(F)F)s4)n3c2)CC1] | 8-chloro-3-(5-(difluoromethyl)-1,3,4-thiadiazol-2- yl)-N-(1-methylcyclopropyl)indolizine-6- sulfonamide |
| 117 | 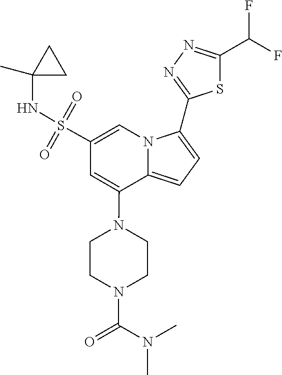 [SMILES: CN(C)C(=O)N1CCN(c2cc(S(=O)(=O)NC3(C)CC3)cn3c(-c4nnc(C(F)F)s4)ccc23)CC1] | 4-(3-(5-(difluoromethyl)-1,3,4-thiadiazol-2-yl)-6- (N-(1-methylcyclopropyl)sulfamoyl)indolizin-8-yl)- N,N-dimethylpiperazine-1-carboxamide |
| 118 | 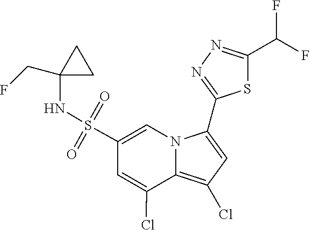 [SMILES: O=S(=O)(NC1(CF)CC1)c1cc(Cl)c2c(Cl)cc(-c3nnc(C(F)F)s3)n2c1] | 1,8-dichloro-3-(5-(difluoromethyl)-1,3,4- thiadiazol-2-yl)-N-(1- (fluoromethyl)cyclopropyl)indolizine-6- sulfonamide |
| 119 | 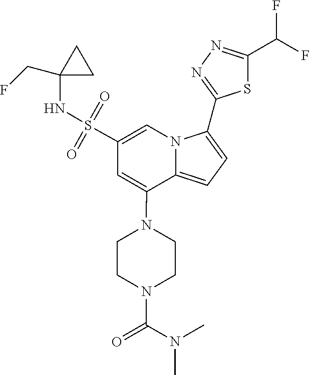 [SMILES: CN(C)C(=O)N1CCN(c2cc(S(=O)(=O)NC3(CF)CC3)cn3c(-c4nnc(C(F)F)s4)ccc23)CC1] | 4-(3-(5-(difluoromethyl)-1,3,4-thiadiazol-2-yl)-6- (N-(1- (fluoromethyl)cyclopropyl)sulfamoyl)indolizin-8- yl)-N,N-dimethylpiperazine-1-carboxamide |
| 120 | 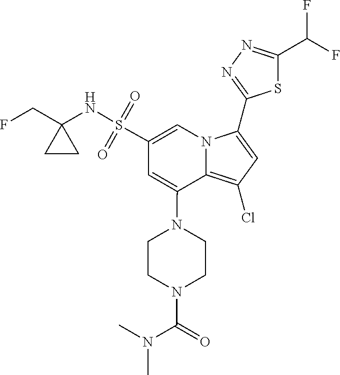 [SMILES: CN(C)C(=O)N1CCN(c2cc(S(=O)(=O)NC3(CF)CC3)cn3c(-c4nnc(C(F)F)s4)cc(Cl)c23)CC1] | 4-(1-chloro-3-(5-(difluoromethyl)-1,3,4-thiadiazol- 2-yl)-6-(N-(1- (fluoromethyl)cyclopropyl)sulfamoyl)indolizin-8- yl)-N,N-dimethylpiperazine-1-carboxamide |
| 121 | 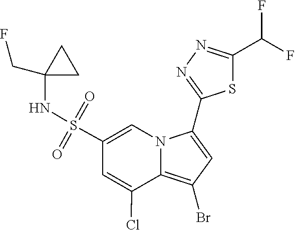 [SMILES: O=S(=O)(NC1(CF)CC1)c1cc(Cl)c2c(Br)cc(-c3nnc(C(F)F)s3)n2c1] | 1-bromo-8-chloro-3-(5-(difluoromethyl)-1,3,4- thiadiazol-2-yl)-N-(1- (fluoromethyl)cyclopropyl)indolizine-6- sulfonamide |
| 122 | 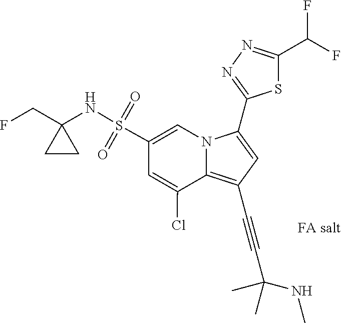 [SMILES: CNC(C)(C)C#Cc1cc(-c2nnc(C(F)F)s2)n2cc(S(=O)(=O)NC3(CF)CC3)cc(Cl)c12] | 8-chloro-3-(5-(difluoromethyl)-1,3,4-thiadiazol-2- yl)-N-(1-(fluoromethyl)cyclopropyl)-1-(3-methyl-3- (methylamino)but-1-yn-1-yl)indolizine-6- sulfonamide formate |
| 123 | 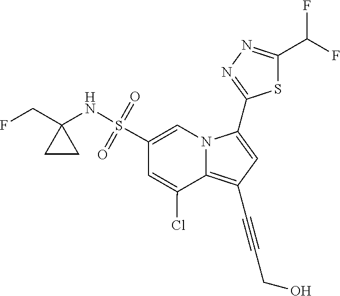 [SMILES: O=S(=O)(NC1(CF)CC1)c1cc(Cl)c2c(C#CCO)cc(-c3nnc(C(F)F)s3)n2c1] | 8-chloro-3-(5-(difluoromethyl)-1,3,4-thiadiazol-2- yl)-N-(1-(fluoromethyl)cyclopropyl)-1-(3- hydroxyprop-1-yn-1-yl)indolizine-6-sulfonamide |
| 124 | 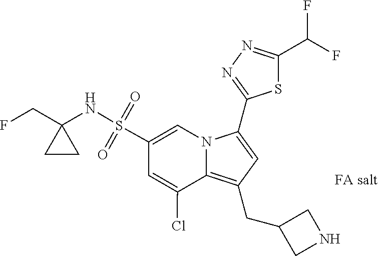 [SMILES: O=S(=O)(NC1(CF)CC1)c1cc(Cl)c2c(CC3CNC3)cc(-c3nnc(C(F)F)s3)n2c1] | 1-(azetidin-3-ylmethyl)-8-chloro-3-(5- (difluoromethyl)-1,3,4-thiadiazol-2-yl)-N-(1- (fluoromethyl)cyclopropyl)indolizine-6- sulfonamide formate |
| 125 | 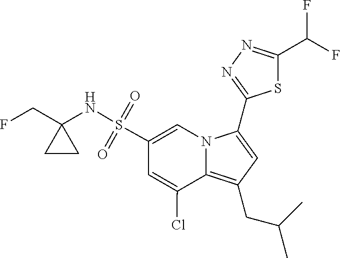 [SMILES: CC(C)Cc1cc(-c2nnc(C(F)F)s2)n2cc(S(=O)(=O)NC3(CF)CC3)cc(Cl)c12] | 8-chloro-3-(5-(difluoromethyl)-1,3,4-thiadiazol-2- yl)-N-(1-(fluoromethyl)cyclopropyl)-1- isobutylindolizine-6-sulfonamide |
| 126 | 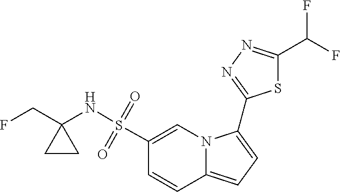 [SMILES: O=S(=O)(NC1(CF)CC1)c1ccc2ccc(-c3nnc(C(F)F)s3)n2c1] | 3-(5-(difluoromethyl)-1,3,4-thiadiazol-2-yl)-N-(1- (fluoromethyl)cyclopropyl)indolizine-6- sulfonamide |
| 127 | 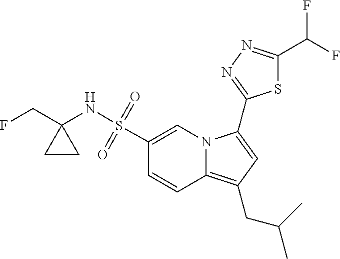 [SMILES: CC(C)Cc1cc(-c2nnc(C(F)F)s2)n2cc(S(=O)(=O)NC3(CF)CC3)ccc12] | 3-(5-(difluoromethyl)-1,3,4-thiadiazol-2-yl)-N-(1- (fluoromethyl)cyclopropyl)-1-isobutylindolizine-6- sulfonamide |
| 128 | 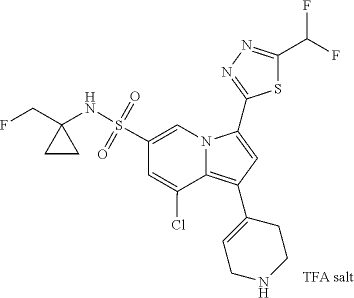 [SMILES: O=S(=O)(NC1(CF)CC1)c1cc(Cl)c2c(C3=CCNCC3)cc(-c3nnc(C(F)F)s3)n2c1] | 8-chloro-3-(5-(difluoromethyl)-1,3,4-thiadiazol-2- yl)-N-(1-(fluoromethyl)cyclopropyl)-1-(1,2,3,6- tetrahydropyridin-4-yl)indolizine-6-sulfonamide 2,2,2-trifluoroacetate |
| 129 | 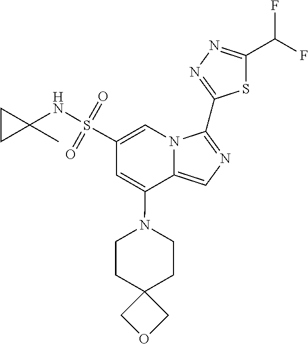 [SMILES: CC1(NS(=O)(=O)c2cc(N3CCC4(CC3)COC4)c3cnc(-c4nnc(C(F)F)s4)n3c2)CC1] | 3-(5-(difluoromethyl)-1,3,4-thiadiazol-2-yl)-N-(1- methylcyclopropyl)-8-(2-oxa-7- azaspiro[3.5]nonan-7-yl)imidazo[1,5-a]pyridine- 6-sulfonamide |
| 130 | 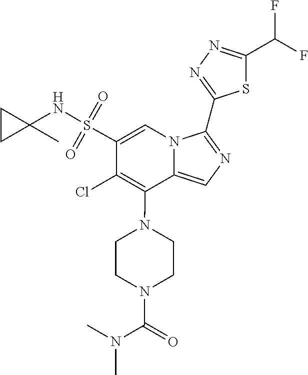 [SMILES: CN(C)C(=O)N1CCN(c2c(Cl)c(S(=O)(=O)NC3(C)CC3)cn3c(-c4nnc(C(F)F)s4)ncc23)CC1] | 4-(7-chloro-3-(5-(difluoromethyl)-1,3,4-thiadiazol- 2-yl)-6-(N-(1- methylcyclopropyl)sulfamoyl)imidazo[1,5- a]pyridin-8-yl)-N,N-dimethylpiperazine-1- carboxamide |
| 131 | 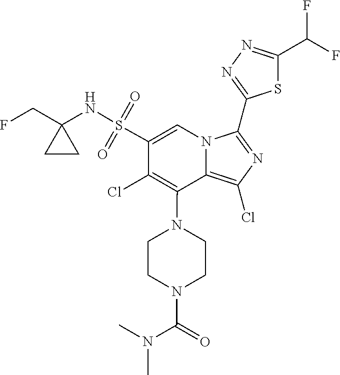 [SMILES: CN(C)C(=O)N1CCN(c2c(Cl)c(S(=O)(=O)NC3(CF)CC3)cn3c(-c4nnc(C(F)F)s4)nc(Cl)c23)CC1] | 4-(1,7-dichloro-3-(5-(difluoromethyl)-1,3,4- thiadiazol-2-yl)-6-(N-(1- (fluoromethyl)cyclopropyl)sulfamoyl)imidazo[1,5- a]pyridin-8-yl)-N,N-dimethylpiperazine-1- carboxamide |
| 132 | 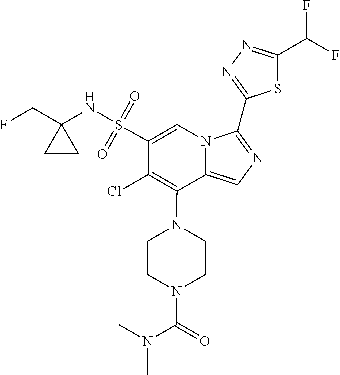 [SMILES: CN(C)C(=O)N1CCN(c2c(Cl)c(S(=O)(=O)NC3(CF)CC3)cn3c(-c4nnc(C(F)F)s4)ncc23)CC1] | 4-(7-chloro-3-(5-(difluoromethyl)-1,3,4-thiadiazol- 2-yl)-6-(N-(1- (fluoromethyl)cyclopropyl)sulfamoyl)imidazo[1,5- a]pyridin-8-yl)-N,N-dimethylpiperazine-1- carboxamide |
| 133.a | 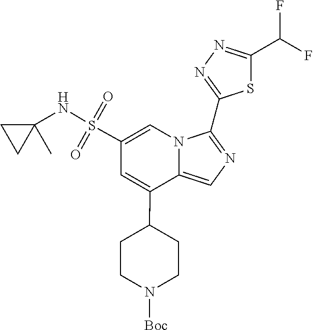 [SMILES: CC1(NS(=O)(=O)c2cc(C3CCN(C(=O)OC(C)(C)C)CC3)c3cnc(-c4nnc(C(F)F)s4)n3c2)CC1] | tert-butyl4-(3-(5-(difluoromethyl)-1,3,4-thiadiazol- 2-yl)-6-(N-(1- methylcyclopropyl)sulfamoyl)imidazo[1,5- a]pyridin-8-yl)piperidine-1-carboxylate |
| 133 | 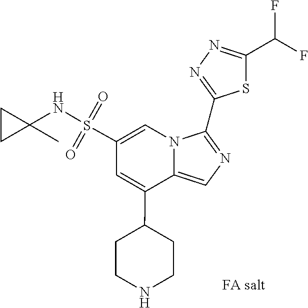 [SMILES: CC1(NS(=O)(=O)c2cc(C3CCNCC3)c3cnc(-c4nnc(C(F)F)s4)n3c2)CC1] | 3-(5-(difluoromethyl)-1,3,4-thiadiazol-2-yl)-N-(1- methylcyclopropyl)-8-(piperidin-4-yl)imidazo[1,5- a]pyridine-6-sulfonamide formate |
| 134 | 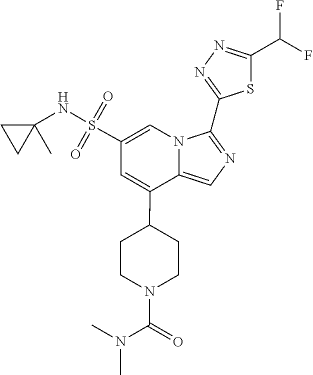 [SMILES: CN(C)C(=O)N1CCC(c2cc(S(=O)(=O)NC3(C)CC3)cn3c(-c4nnc(C(F)F)s4)ncc23)CC1] | 4-(3-(5-(difluoromethyl)-1,3,4-thiadiazol-2-yl)-6- (N-(1-methylcyclopropyl)sulfamoyl)imidazo[1,5- a]pyridin-8-yl)-N,N-dimethylpiperidine-1- carboxamide |
| 135 | 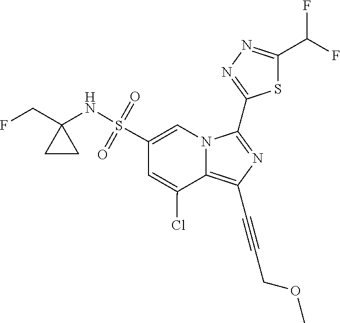 [SMILES: COCC#Cc1nc(-c2nnc(C(F)F)s2)n2cc(S(=O)(=O)NC3(CF)CC3)cc(Cl)c12] | 8-chloro-3-(5-(difluoromethyl)-1,3,4-thiadiazol-2- yl)-N-(1-(fluoromethyl)cyclopropyl)-1-(3- methoxyprop-1-yn-1-yl)imidazo[1,5-a]pyridine-6- sulfonamide |
| 136 | 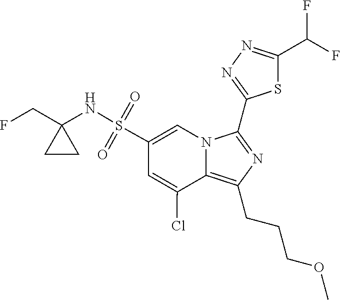 [SMILES: COCCCc1nc(-c2nnc(C(F)F)s2)n2cc(S(=O)(=O)NC3(CF)CC3)cc(Cl)c12] | 8-chloro-3-(5-(difluoromethyl)-1,3,4-thiadiazol-2- yl)-N-(1-(fluoromethyl)cyclopropyl)-1-(3- methoxypropyl)imidazo[1,5-a]pyridine-6- sulfonamide |
| 137 | 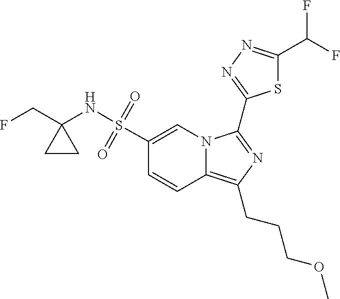 [SMILES: COCCCc1nc(-c2nnc(C(F)F)s2)n2cc(S(=O)(=O)NC3(CF)CC3)ccc12] | 3-(5-(difluoromethyl)-1,3,4-thiadiazol-2-yl)-N-(1- (fluoromethyl)cyclopropyl)-1-(3- methoxypropyl)imidazo[1,5-a]pyridine-6- sulfonamide |
| 138 | 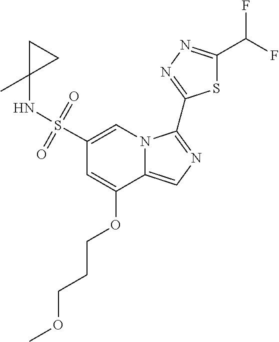 [SMILES: COCCCOc1cc(S(=O)(=O)NC2(C)CC2)cn2c(-c3nnc(C(F)F)s3)ncc12] | 3-(5-(difluoromethyl)-1,3,4-thiadiazol-2-yl)-8-(3- methoxypropoxy)-N-(1- methylcyclopropyl)imidazo[1,5-a]pyridine-6- sulfonamide |
| 139 | 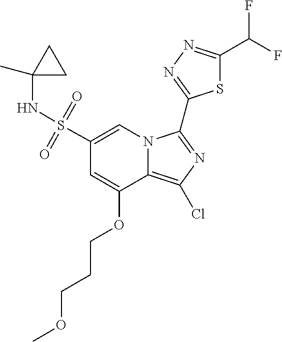 [SMILES: COCCCOc1cc(S(=O)(=O)NC2(C)CC2)cn2c(-c3nnc(C(F)F)s3)nc(Cl)c12] | 1-chloro-3-(5-(difluoromethyl)-1,3,4-thiadiazol-2- yl)-8-(3-methoxypropoxy)-N-(1- methylcyclopropyl)imidazo[1,5-a]pyridine-6- sulfonamide |
| 140 | 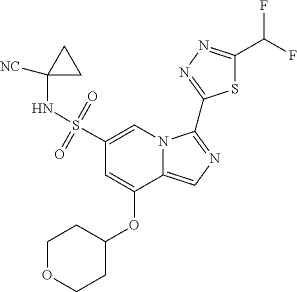 [SMILES: N#CC1(NS(=O)(=O)c2cc(OC3CCOCC3)c3cnc(-c4nnc(C(F)F)s4)n3c2)CC1] | 3-(5-(difluoromethyl)-1,3,4-thiadiazol-2-yl)-N-(1- methylcyclopropyl)-8-((tetrahydro-2H-pyran-4- yl)oxy)imidazo[1,5-a]pyridine-6-sulfonamide |
| 141 | 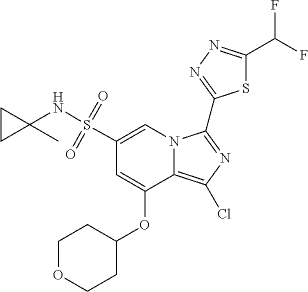 [SMILES: CC1(NS(=O)(=O)c2cc(OC3CCOCC3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-((tetrahydro-2H- pyran-4-yl)oxy)imidazo[1,5-a]pyridine-6- sulfonamide |
| 142 | 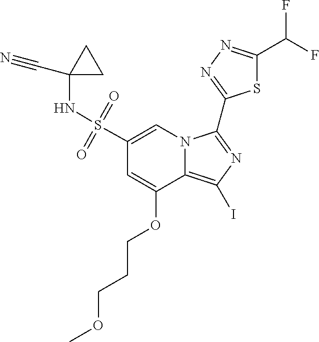 [SMILES: COCCCOc1cc(S(=O)(=O)NC2(C#N)CC2)cn2c(-c3nnc(C(F)F)s3)nc(I)c12] | N-(1-cyanocyclopropyl)-3-(5-(difluoromethyl)- 1,3,4-thiadiazol-2-yl)-1-iodo-8-(3- methoxypropoxy)imidazo[1,5-a]pyridine-6- sulfonamide |
| 143 | 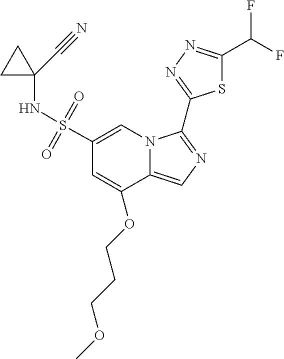 [SMILES: COCCCOc1cc(S(=O)(=O)NC2(C#N)CC2)cn2c(-c3nnc(C(F)F)s3)ncc12] | N-(1-cyanocyclopropyl)-3-(5-(difluoromethyl)- 1,3,4-thiadiazol-2-yl)-8-(3- methoxypropoxy)imidazo[1,5-a]pyridine-6- sulfonamide |
| 146a | 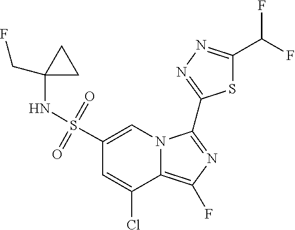 [SMILES: O=S(=O)(NC1(CF)CC1)c1cc(Cl)c2c(F)nc(-c3nnc(C(F)F)s3)n2c1] | 8-chloro-3-(5-(difluoromethyl)-1,3,4-thiadiazol-2- yl)-1-fluoro-N-(1- (fluoromethyl)cyclopropyl)imidazo[1,5-a]pyridine- 6-sulfonamide |
| 146 | 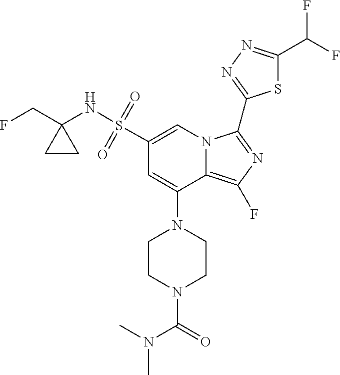 [SMILES: CN(C)C(=O)N1CCN(c2cc(S(=O)(=O)NC3(CF)CC3)cn3c(-c4nnc(C(F)F)s4)nc(F)c23)CC1] | 4-(3-(5-(difluoromethyl)-1,3,4-thiadiazol-2-yl)-1- fluoro-6-(N-(1- (fluoromethyl)cyclopropyl)sulfamoyl)imidazo[1,5- a]pyridin-8-yl)-N,N-dimethylpiperazine-1- carboxamide |
| 147 | 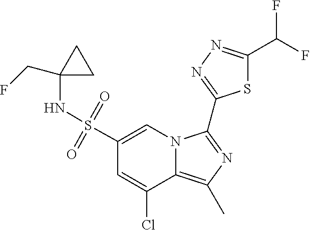 [SMILES: Cc1nc(-c2nnc(C(F)F)s2)n2cc(S(=O)(=O)NC3(CF)CC3)cc(Cl)c12] | 8-chloro-3-(5-(difluoromethyl)-1,3,4-thiadiazol-2- yl)-N-(1-(fluoromethyl)cyclopropyl)-1- methylimidazo[1,5-a]pyridine-6-sulfonamide |
| 148 | 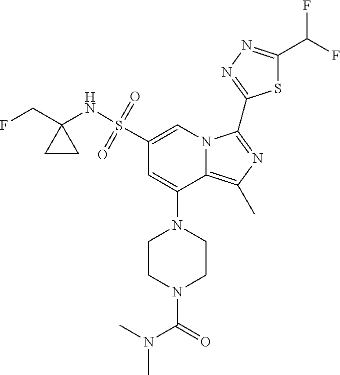 [SMILES: Cc1nc(-c2nnc(C(F)F)s2)n2cc(S(=O)(=O)NC3(CF)CC3)cc(N3CCN(C(=O)N(C)C)CC3)c12] | 4-(3-(5-(difluoromethyl)-1,3,4-thiadiazol-2-yl)-6- (N-(1-(fluoromethyl)cyclopropyl)sulfamoyl)-1- methylimidazo[1,5-a]pyridin-8-yl)-N,N- dimethylpiperazine-1-carboxamide |
| 149 | 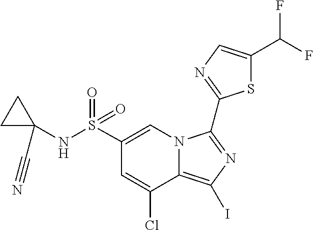 [SMILES: N#CC1(NS(=O)(=O)c2cc(Cl)c3c(I)nc(-c4ncc(C(F)F)s4)n3c2)CC1] | 8-chloro-N-(1-cyanocyclopropyl)-3-(5- (difluoromethyl)thiazol-2-yl)-1-iodoimidazo[1,5- a]pyridine-6-sulfonamide |
| 150 | 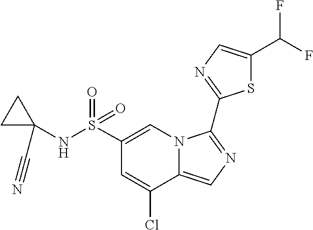 [SMILES: N#CC1(NS(=O)(=O)c2cc(Cl)c3cnc(-c4ncc(C(F)F)s4)n3c2)CC1] | 8-chloro-N-(1-cyanocyclopropyl)-3-(5- (difluoromethyl)thiazol-2-yl)imidazo[1,5- a]pyridine-6-sulfonamide |
| 151 | 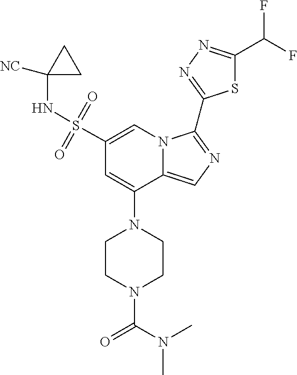 [SMILES: CN(C)C(=O)N1CCN(c2cc(S(=O)(=O)NC3(C#N)CC3)cn3c(-c4nnc(C(F)F)s4)ncc23)CC1] | 4-(6-(N-(1-cyanocyclopropyl)sulfamoyl)-3-(5- (difluoromethyl)thiazol-2-yl)imidazo[1,5-a]pyridin- 8-yl)-N,N-dimethylpiperazine-1-carboxamide |
| 152 | 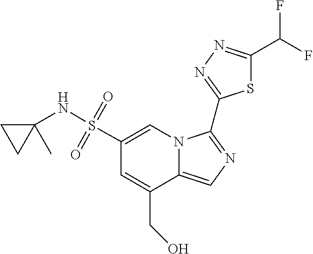 [SMILES: CC1(NS(=O)(=O)c2cc(CO)c3cnc(-c4nnc(C(F)F)s4)n3c2)CC1] | 3-(5-(difluoromethyl)-1,3,4-thiadiazol-2-yl)-8- (hydroxymethyl)-N-(1- methylcyclopropyl)imidazo[1,5-a]pyridine-6- sulfonamide |
| 153 | 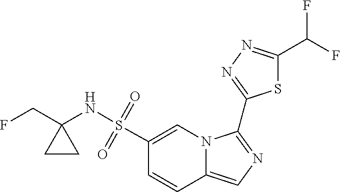 [SMILES: O=S(=O)(NC1(CF)CC1)c1ccc2cnc(-c3nnc(C(F)F)s3)n2c1] | 3-(5-(difluoromethyl)-1,3,4-thiadiazol-2-yl)-N-(1- (fluoromethyl)cyclopropyl)imidazo[1,5-a]pyridine- 6-sulfonamide |
| 154 | 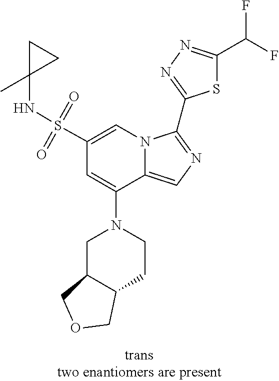 [SMILES: CC1(NS(=O)(=O)c2cc(N3CC[C@@H]4COC[C@H]4C3)c3cnc(-c4nnc(C(F)F)s4)n3c2)CC1] | 3-(5-(difluoromethyl)-1,3,4-thiadiazol-2-yl)-8- ((trans)-hexahydrofuro[3,4-c]pyridin-5(3H)-yl)-N- (1-methylcyclopropyl)imidazo[1,5-a]pyridine-6- sulfonamide |
| 155 | 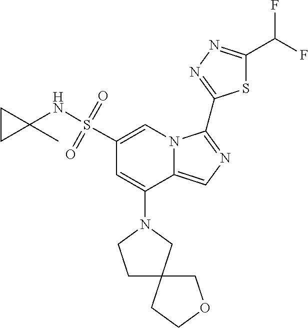 [SMILES: CC1(NS(=O)(=O)c2cc(N3CCC4(CCOC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(2-oxa-7- azaspiro[4.4]nonan-7-yl)imidazo[1,5-a]pyridine- 6-sulfonamide |
| 156 | 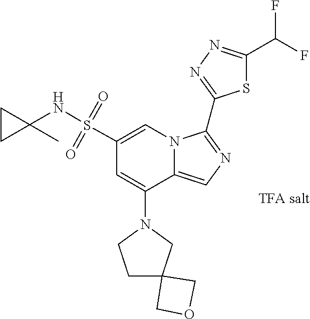 [SMILES: CC1(NS(=O)(=O)c2cc(N3CCC4(COC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(2-oxa-6- azaspiro[3.4]octan-6-yl)imidazo[1,5-a]pyridine-6- sulfonamide 2,2,2-trifluoroacetate |
| 157 | 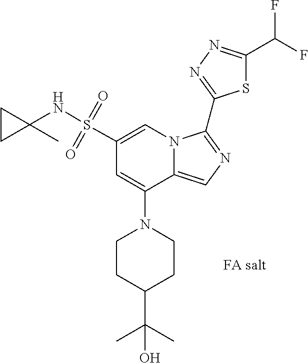 [SMILES: CC1(NS(=O)(=O)c2cc(N3CCC(C(C)(C)O)CC3)c3cnc(-c4nnc(C(F)F)s4)n3c2)CC1] | 3-(5-(difluoromethyl)-1,3,4-thiadiazol-2-yl)-8-(4- (2-hydroxypropan-2-yl)piperidin-1-yl)-N-(1- methylcyclopropyl)imidazo[1,5-a]pyridine-6- sulfonamide formate |
| 158 | 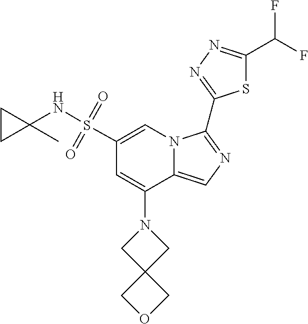 [SMILES: CC1(NS(=O)(=O)c2cc(N3CC4(COC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(2-oxa-6- azaspiro[3.3]heptan-6-yl)imidazo[1,5-a]pyridine- 6-sulfonamide |
| 159 | 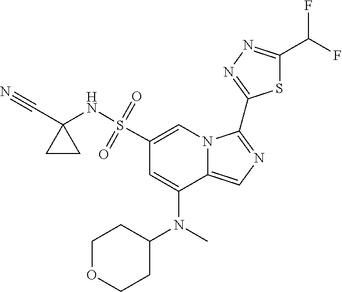 [SMILES: CN(c1cc(S(=O)(=O)NC2(C#N)CC2)cn2c(-c3nnc(C(F)F)s3)ncc12)C1CCOCC1] | N-(1-cyanocyclopropyl)-3-(5-(difluoromethyl)- 1,3,4-thiadiazol-2-yl)-8-(methyl(tetrahydro-2H- pyran-4-yl)amino)imidazo[1,5-a]pyridine-6- sulfonamide |
| 160 | 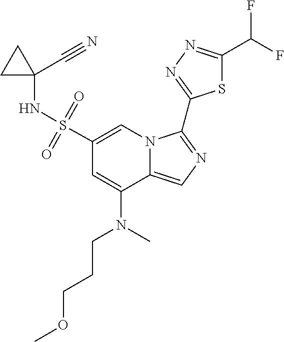 [SMILES: COCCCN(C)c1cc(S(=O)(=O)NC2(C#N)CC2)cn2c(-c3nnc(C(F)F)s3)ncc12] | N-(1-cyanocyclopropyl)-3-(5-(difluoromethyl)- 1,3,4-thiadiazol-2-yl)-8-((3- methoxypropyl)(methyl)amino)imidazo[1,5- a]pyridine-6-sulfonamide |
| 161 | 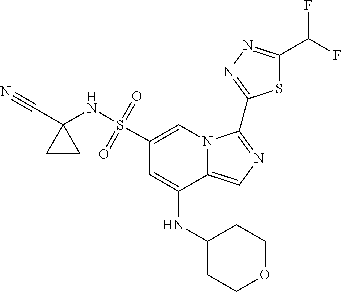 [SMILES: N#CC1(NS(=O)(=O)c2cc(NC3CCOCC3)c3cnc(-c4nnc(C(F)F)s4)n3c2)CC1] | N-(1-cyanocyclopropyl)-3-(5-(difluoromethyl)- 1,3,4-thiadiazol-2-yl)-8-((tetrahydro-2H-pyran-4- yl)amino)imidazo[1,5-a]pyridine-6-sulfonamide |
| 162 | 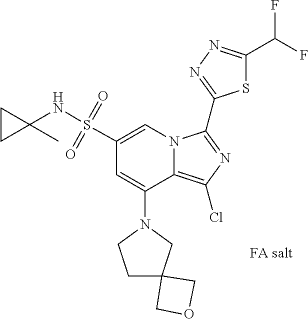 [SMILES: CC1(NS(=O)(=O)c2cc(N3CCC4(COC4)C3)c3c(Cl)nc(-c4nnc(C(F)F)s4)n3c2)CC1] | 8-(4-acetylpiperazin-1-yl)-3-(5-(difluoromethyl)- 1,3,4-thiadiazol-2-yl)-N-(1- methylcyclopropyl)imidazo[1,5-a]pyridine-6- sulfonamide formate |
| 163 | 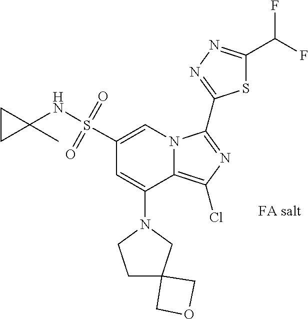 [SMILES: CC1(NS(=O)(=O)c2cc(N3CCC4(COC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2-oxa-6- azaspiro[3.4]octan-6-yl)imidazo[1,5-a]pyridine-6- sulfonamide formate |
| 164 | 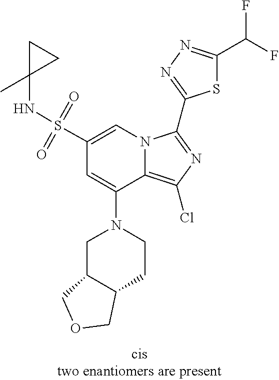 [SMILES: CC1(NS(=O)(=O)c2cc(N3CC[C@@H]4COC[C@@H]4C3)c3c(Cl)nc(-c4nnc(C(F)F)s4)n3c2)CC1] | 1-chloro-3-(5-(difluoromethyl)-1,3,4-thiadiazol-2- yl)-8-((cis)-hexahydrofuro[3,4-c]pyridin-5(3H)-yl)- N-(1-methylcyclopropyl)imidazo[1,5-a]pyridine-6- sulfonamide |
| 165 | 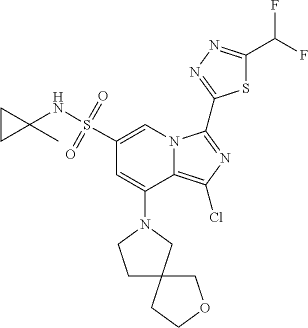 [SMILES: CC1(NS(=O)(=O)c2cc(N3CCC4(CCOC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2-oxa-7- azaspiro[4.4]nonan-7-yl)imidazo[1,5-a]pyridine- 6-sulfonamide |
| 166 | 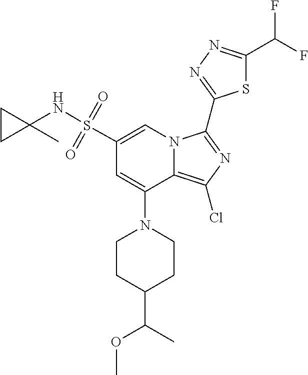 [SMILES: COC(C)C1CCN(c2cc(S(=O)(=O)NC3(C)CC3)cn3c(-c4nnc(C(F)F)s4)nc(Cl)c23)CC1] | 1-chloro-3-(5-(difluoromethyl)-1,3,4-thiadiazol-2- yl)-8-(4-(1-methoxyethyl)piperidin-1-yl)-N-(1- methylcyclopropyl)imidazo[1,5-a]pyridine-6- sulfonamide |
| 168 | 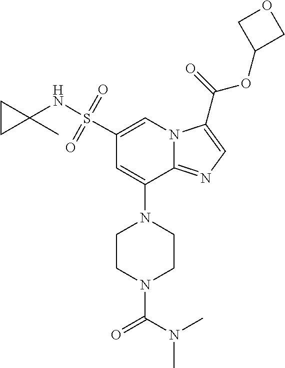 [SMILES: CN(C)C(=O)N1CCN(c2cc(S(=O)(=O)NC3(C)CC3)cn3c(C(=O)OC4COC4)cnc23)CC1] | oxetan-3-yl 8-(4-(dimethylcarbamoyl)piperazin-1- yl)-6-(N-(1- methylcyclopropyl)sulfamoyl)imidazo[1,2- a]pyridine-3-carboxylate |
| 169 | 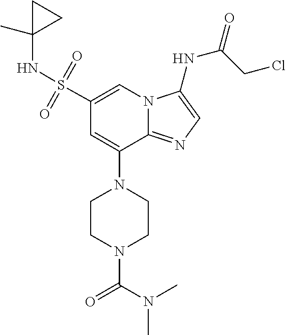 [SMILES: CN(C)C(=O)N1CCN(c2cc(S(=O)(=O)NC3(C)CC3)cn3c(NC(=O)CCl)cnc23)CC1] | 4-(3-(2-chloroacetamido)-6-(N-(1- methylcyclopropyl)sulfamoyl)imidazo[1,2- a]pyridin-8-yl)-N,N-dimethylpiperazine-1- carboxamide |
| 170 | 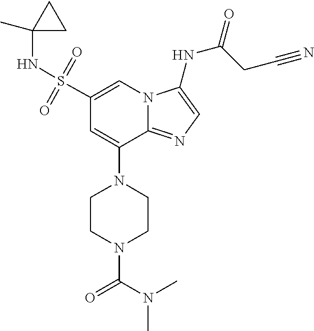 [SMILES: CN(C)C(=O)N1CCN(c2cc(S(=O)(=O)NC3(C)CC3)cn3c(NC(=O)CC#N)cnc23)CC1] | 4-(3-(2-cyanoacetamido)-6-(N-(1- methylcyclopropyl)sulfamoyl)imidazo[1,2- a]pyridin-8-yl)-N,N-dimethylpiperazine-1- carboxamide |
| 171 | 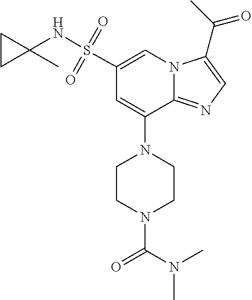 [SMILES: CC(=O)c1cnc2c(N3CCN(C(=O)N(C)C)CC3)cc(S(=O)(=O)NC3(C)CC3)cn12] | 4-(3-acetyl-6-(N-(1- methylcyclopropyl)sulfamoyl)imidazo[1,2- a]pyridin-8-yl)-N,N-dimethylpiperazine-1- carboxamide |
| 172 | 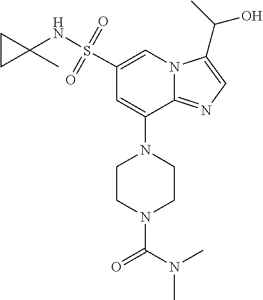 [SMILES: CC(O)c1cnc2c(N3CCN(C(=O)N(C)C)CC3)cc(S(=O)(=O)NC3(C)CC3)cn12] | 4-(3-(1-hydroxyethyl)-6-(N-(1- methylcyclopropyl)sulfamoyl)imidazo[1,2- a]pyridin-8-yl)-N,N-dimethylpiperazine-1- carboxamide |
| 173 | 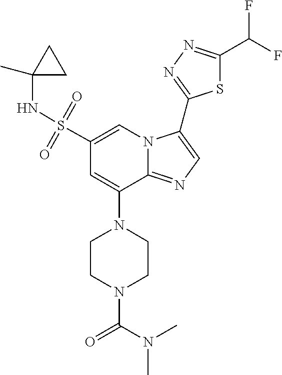 [SMILES: CN(C)C(=O)N1CCN(c2cc(S(=O)(=O)NC3(C)CC3)cn3c(-c4nnc(C(F)F)s4)cnc23)CC1] | 3-(5-(difluoromethyl)-1,3,4-thiadiazol-2-yl)-8-(4- isobutyrylpiperazin-1-yl)-N-(1- methylcyclopropyl)imidazo[1,2-a]pyridine-6- sulfonamide |
| 174 | 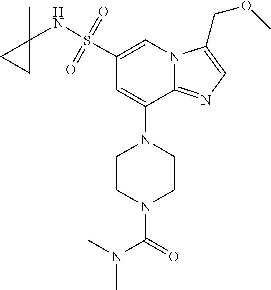 [SMILES: COCc1cnc2c(N3CCN(C(=O)N(C)C)CC3)cc(S(=O)(=O)NC3(C)CC3)cn12] | 4-(3-(methoxymethyl)-6-(N-(1- methylcyclopropyl)sulfamoyl)imidazo[1,2- a]pyridin-8-yl)-N,N-dimethylpiperazine-1- carboxamide |
| 175 | 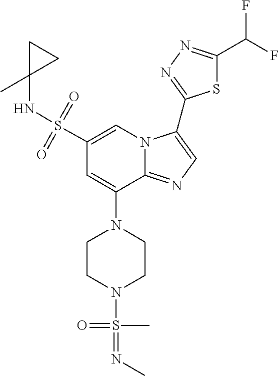 [SMILES: CN=S(C)(=O)N1CCN(c2cc(S(=O)(=O)NC3(C)CC3)cn3c(-c4nnc(C(F)F)s4)cnc23)CC1] | 3-(5-(difluoromethyl)-1,3,4-thiadiazol-2-yl)-8-(4- (N,S-dimethylsulfonimidoyl)piperidin-1-yl)-N-(1- methylcyclopropyl)imidazo[1,2-a]pyridine-6- sulfonamide |
| 176a | 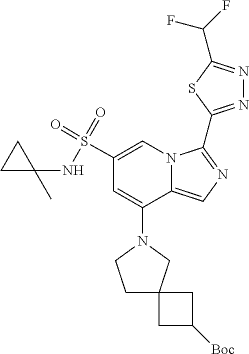 [SMILES: CC1(NS(=O)(=O)c2cc(N3CCC4(CC(C(=O)OC(C)(C)C)C4)C3)c3cnc(-c4nnc(C(F)F)s4)n3c2)CC1] | tert-butyl 6-(3-(5-(difluoromethyl)-1,3,4-thiadiazol- 2-yl)-6-(N-(1- methylcyclopropyl)sulfamoyl)imidazo[1,5- a]pyridin-8-yl)-2,6-diazaspiro[3.4]octane-2- carboxylate |
| 176 | 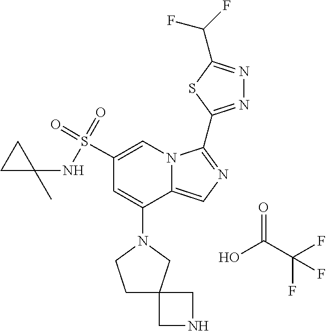 [SMILES: CC1(NS(=O)(=O)c2cc(N3CCC4(CNC4)C3)c3cnc(-c4nnc(C(F)F)s4)n3c2)CC1 ; O=C(O)C(F)(F)F] | 3-(5-(difluoromethyl)-1,3,4-thiadiazol-2-yl)-N-(1- methylcyclopropyl)-8-(2,6-diazaspiro[3.4]octan-6- yl)imidazo[1,5-a]pyridine-6-sulfonamide 2,2,2- trifluoroacetate |
| 177a | 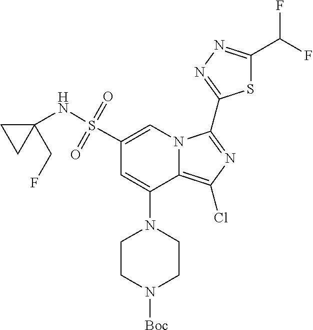 [SMILES: CC(C)(C)OC(=O)N1CCN(c2cc(S(=O)(=O)NC3(CF)CC3)cn3c(-c4nnc(C(F)F)s4)nc(Cl)c23)CC1] | tert-butyl 4-(1-chloro-3-(5-(difluoromethyl)-1,3,4- thiadiazol-2-yl)-6-(N-(1- (fluoromethyl)cyclopropyl)sulfamoyl)imidazo[1,5- a]pyridin-8-yl)piperazine-1-carboxylate |
| 177b | 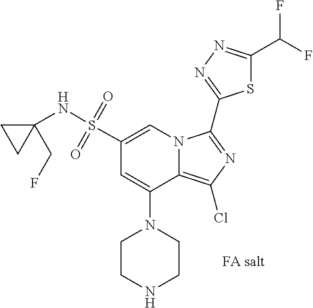 [SMILES: O=S(=O)(NC1(CF)CC1)c1cc(N2CCNCC2)c2c(Cl)nc(-c3nnc(C(F)F)s3)n2c1] | 1-chloro-3-(5-(difluoromethyl)-1,3,4-thiadiazol-2- yl)-N-(1-(fluoromethyl)cyclopropyl)-8-(piperazin- 1-yl)imidazo[1,5-a]pyridine-6-sulfonamide formate |
| 177 | 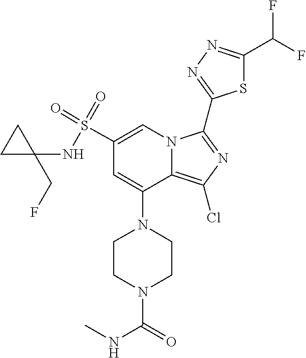 [SMILES: CNC(=O)N1CCN(c2cc(S(=O)(=O)NC3(CF)CC3)cn3c(-c4nnc(C(F)F)s4)nc(Cl)c23)CC1] | 4-(1-chloro-3-(5-(difluoromethyl)-1,3,4-thiadiazol- 2-yl)-6-(N-(1- (fluoromethyl)cyclopropyl)sulfamoyl)imidazo[1,5- a]pyridin-8-yl)-N-methylpiperazine-1- carboxamide |
| 178a | 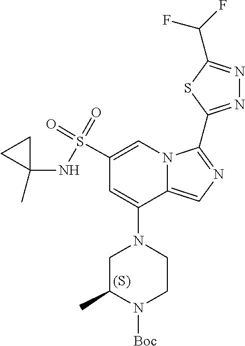 | (S)-tert-butyl 4-(3-(5-(difluoromethyl)-1,3,4- thiadiazol-2-yl)-6-(N-(1- methylcyclopropyl)sulfamoyl)imidazo[1,5- a]pyridin-8-yl)-2-methylpiperazine-1-carboxylate |
| 178 | 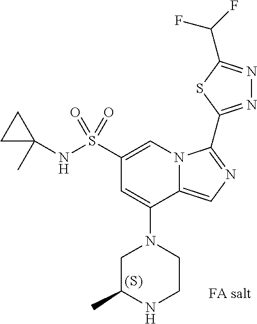 | (S)-3-(5-(difluoromethyl)-1,3,4-thiadiazol-2-yl)-N- (1-methylcyclopropyl)-8-(3-methylpiperazin-1- yl)imidazo[1,5-a]pyridine-6-sulfonamide formate |
| 179 | 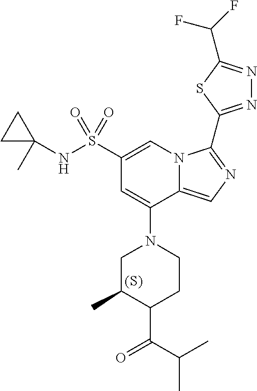 | (S)-3-(5-(difluoromethyl)-1,3,4-thiadiazol-2-yl)-8- (4-isobutyryl-3-methylpiperazin-1-yl)-N-(1- methylcyclopropyl)imidazo[1,5-a]pyridine-6- sulfonamide |
| 180a | 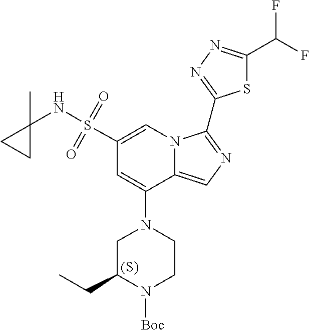 | (S)-tert-butyl 4-(3-(5-(difluoromethyl)-1,3,4- thiadiazol-2-yl)-6-(N-(1- methylcyclopropyl)sulfamoyl)imidazo[1,5- a]pyridin-8-yl)-2-ethylpiperazine-1-carboxylate |
| 180 | 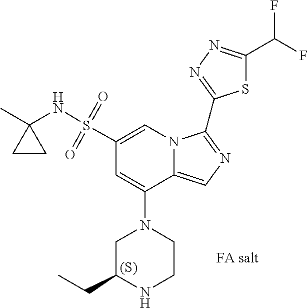 | (S)-3-(5-(difluoromethyl)-1,3,4-thiadiazol-2-yl)-8- (3-ethylpiperazin-1-yl)-N-(1- methylcyclopropyl)imidazo[1,5-a]pyridine-6- sulfonamide formate |
| 181a | 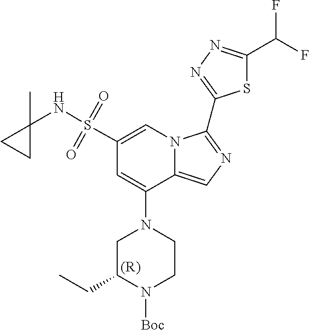 | (R)-tert-butyl 4-(3-(5-(difluoromethyl)-1,3,4- thiadiazol-2-yl)-6-(N-(1- methylcyclopropyl)sulfamoyl)imidazo[1,5- a]pyridin-8-yl)-2-ethylpiperazine-1-carboxylate |
| 181 | 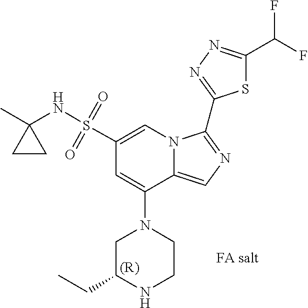 | (R)-3-(5-(difluoromethyl)-1,3,4-thiadiazol-2-yl)-8- (3-ethylpiperazin-1-yl)-N-(1- methylcyclopropyl)imidazo[1,5-a]pyridine-6- sulfonamide formate |
| 182 | 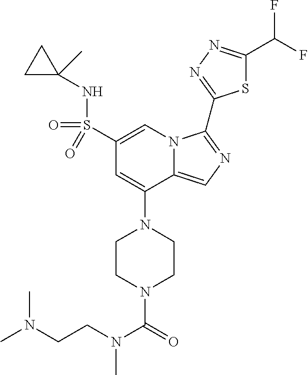 [SMILES: CN(C)CCN(C)C(=O)N1CCN(c2cc(S(=O)(=O)NC3(C)CC3)cn3c(-c4nnc(C(F)F)s4)ncc23)CC1] | 4-(3-(5-(difluoromethyl)-1,3,4-thiadiazol-2-yl)-6- (N-(1-methylcyclopropyl)sulfamoyl)imidazo[1,5- a]pyridin-8-yl)-N-(2-(dimethylamino)ethyl)-N- methylpiperazine-1-carboxamide |
| 183a | 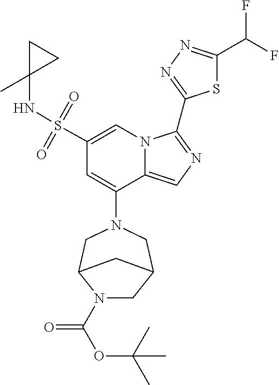 [SMILES: CC1(NS(=O)(=O)c2cc(N3CC4CC(C3)N(C(=O)OC(C)(C)C)C4)c3cnc(-c4nnc(C(F)F)s4)n3c2)CC1] | tert-butyl 3-(3-(5-(difluoromethyl)-1,3,4-thiadiazol- 2-yl)-6-(N-(1- methylcyclopropyl)sulfamoyl)imidazo[1,5- a]pyridin-8-yl)-3,6-diazabicyclo[3.2.1]octane-6- carboxylate |
| 183 | 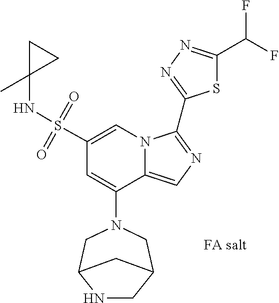 [SMILES: CC1(NS(=O)(=O)c2cc(N3CC4CNC(C4)C3)c3cnc(-c4nnc(C(F)F)s4)n3c2)CC1] | 8-(3,6-diazabicyclo[3.2.1]octan-3-yl)-3-(5- (difluoromethyl)-1,3,4-thiadiazol-2-yl)-N-(1- methylcyclopropyl)imidazo[1,5-a]pyridine-6- sulfonamide formate |
| 184a | 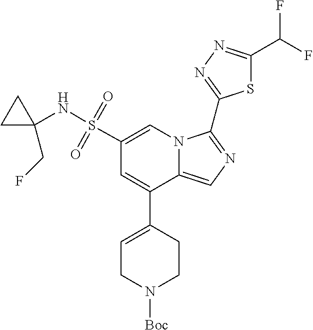 [SMILES: CC(C)(C)OC(=O)N1CC=C(c2cc(S(=O)(=O)NC3(CF)CC3)cn3c(-c4nnc(C(F)F)s4)ncc23)CC1] | tert-butyl 4-(3-(5-(difluoromethyl)-1,3,4-thiadiazol- 2-yl)-6-(N-(1- (fluoromethyl)cyclopropyl)sulfamoyl)imidazo[1,5- a]pyridin-8-yl)-5,6-dihydropyridine-1(2H)- carboxylate |
| 184b | 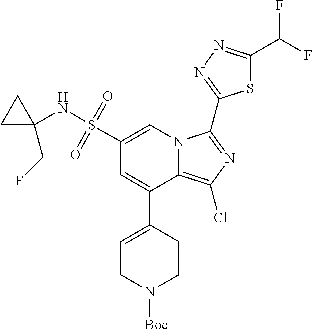 [SMILES: CC(C)(C)OC(=O)N1CC=C(c2cc(S(=O)(=O)NC3(CF)CC3)cn3c(-c4nnc(C(F)F)s4)nc(Cl)c23)CC1] | tert-butyl 4-(1-chloro-3-(5-(difluoromethyl)-1,3,4- thiadiazol-2-yl)-6-(N-(1- (fluoromethyl)cyclopropyl)sulfamoyl)imidazo[1,5- a]pyridin-8-yl)-5,6-dihydropyridine-1(2H)- carboxylate |
| 184 | 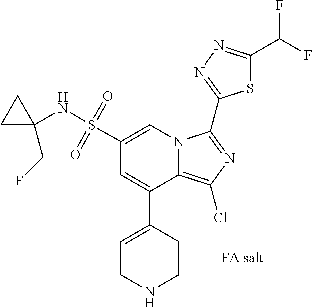 [SMILES: O=S(=O)(NC1(CF)CC1)c1cc(C2=CCNCC2)c2c(Cl)nc(-c3nnc(C(F)F)s3)n2c1] | 1-chloro-3-(5-(difluoromethyl)-1,3,4-thiadiazol-2- yl)-N-(1-(fluoromethyl)cyclopropyl)-8-(1,2,3,6- tetrahydropyridin-4-yl)imidazo[1,5-a]pyridine-6- sulfonamide formate |
| 185 | 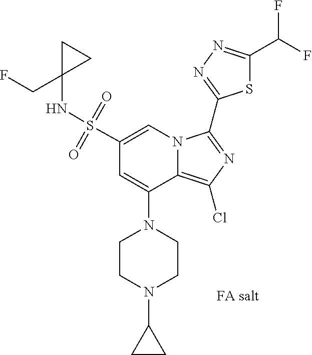 [SMILES: O=S(=O)(NC1(CF)CC1)c1cc(N2CCN(C3CC3)CC2)c2c(Cl)nc(-c3nnc(C(F)F)s3)n2c1] | 1-chloro-8-(4-cyclopropylpiperazin-1-yl)-3-(5- (difluoromethyl)-1,3,4-thiadiazol-2-yl)-N-(1- (fluoromethyl)cyclopropyl)imidazo[1,5-a]pyridine- 6-sulfonamide formate |
| 186a | 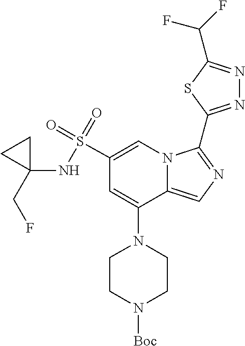 [SMILES: CC(C)(C)OC(=O)N1CCN(c2cc(S(=O)(=O)NC3(CF)CC3)cn3c(-c4nnc(C(F)F)s4)ncc23)CC1] | tert-butyl 4-(3-(5-(difluoromethyl)-1,3,4-thiadiazol- 2-yl)-6-(N-(1- (fluoromethyl)cyclopropyl)sulfamoyl)imidazo[1,5- a]pyridin-8-yl)piperazine-1-carboxylate |
| 187 | 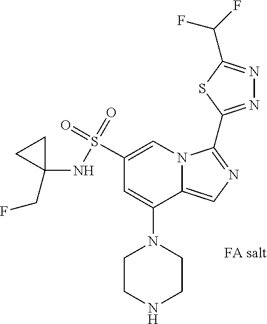 [SMILES: O=S(=O)(NC1(CF)CC1)c1cc(N2CCNCC2)c2cnc(-c3nnc(C(F)F)s3)n2c1] | 3-(5-(difluoromethyl)-1,3,4-thiadiazol-2-yl)-N-(1- (fluoromethyl)cyclopropyl)-8-(piperazin-1- yl)imidazo[1,5-a]pyridine-6-sulfonamide formate |
| 188 | 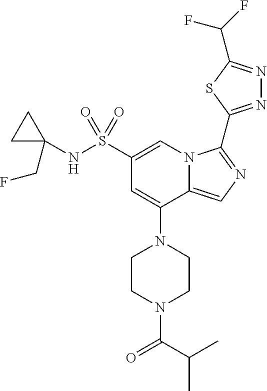 [SMILES: CC(C)C(=O)N1CCN(c2cc(S(=O)(=O)NC3(CF)CC3)cn3c(-c4nnc(C(F)F)s4)ncc23)CC1] | 3-(5-(difluoromethyl)-1,3,4-thiadiazol-2-yl)-N-(1- (fluoromethyl)cyclopropyl)-8-(4- isobutyrylpiperazin-1-yl)imidazo[1,5-a]pyridine-6- sulfonamide |
| 189a | 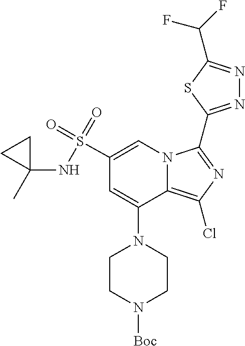 [SMILES: CC1(NS(=O)(=O)c2cc(N3CCN(C(=O)OC(C)(C)C)CC3)c3c(Cl)nc(-c4nnc(C(F)F)s4)n3c2)CC1] | tert-butyl 4-(1-chloro-3-(5-(difluoromethyl)-1,3,4- thiadiazol-2-yl)-6-(N-(1- methylcyclopropyl)sulfamoyl)imidazo[1,5- a]pyridin-8-yl)piperazine-1-carboxylate |
| 189 | 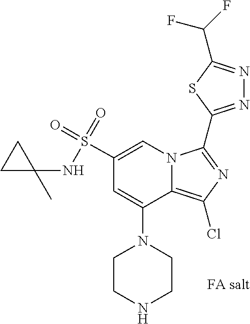 [SMILES: CC1(NS(=O)(=O)c2cc(N3CCNCC3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(piperazin-1- yl)imidazo[1,5-a]pyridine-6-sulfonamide formate |
| 190a | 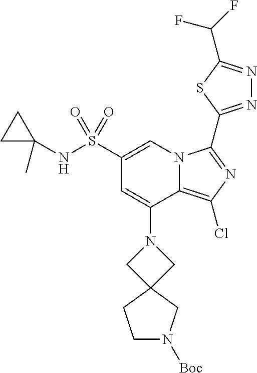 [SMILES: CC1(NS(=O)(=O)c2cc(N3CC4(CCN(C(=O)OC(C)(C)C)C4)C3)c3c(Cl)nc(-c4nnc(C(F)F)s4)n3c2)CC1] | tert-butyl 2-(1-chloro-3-(5-(difluoromethyl)-1,3,4- thiadiazol-2-yl)-6-(N-(1- methylcyclopropyl)sulfamoyl)imidazo[1,5- a]pyridin-8-yl)-2,6-diazaspiro[3.4]octane-6- carboxylate |
| 190 | 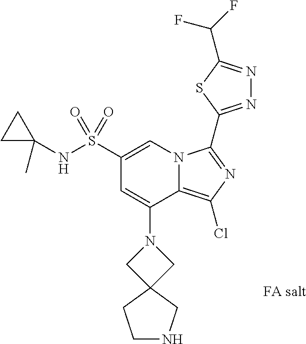 [SMILES: CC1(NS(=O)(=O)c2cc(N3CC4(CCNC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2,6- diazaspiro[3.4]octan-2-yl)imidazo[1,5-a]pyridine- 6-sulfonamide formate |
| 191a | 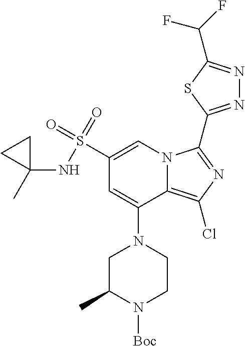 [SMILES: C[C@H]1CN(c2cc(S(=O)(=O)NC3(C)CC3)cn3c(-c4nnc(C(F)F)s4)nc(Cl)c23)CCN1C(=O)OC(C)(C)C] | (S)-tert-butyl 4-(1-chloro-3-(5-(difluoromethyl)- 1,3,4-thiadiazol-2-yl)-6-(N-(1- methylcyclopropyl)sulfamoyl)imidazo[1,5- a]pyridin-8-yl)-2-methylpiperazine-1-carboxylate |
| 191 | 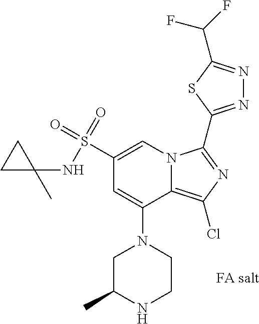 [SMILES: C[C@H]1CN(c2cc(S(=O)(=O)NC3(C)CC3)cn3c(-c4nnc(C(F)F)s4)nc(Cl)c23)CCN1] | (S)-1-chloro-3-(5-(difluoromethyl)-1,3,4- thiadiazol-2-yl)-N-(1-methylcyclopropyl)-8-(3- methylpiperazin-1-yl)imidazo[1,5-a]pyridine-6- sulfonamide formate |
| 192a | 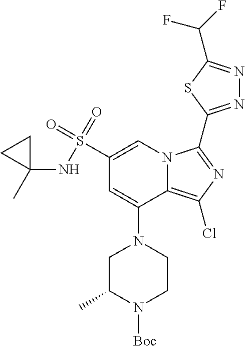 [SMILES: C[C@@H]1CN(c2cc(S(=O)(=O)NC3(C)CC3)cn3c(-c4nnc(C(F)F)s4)nc(Cl)c23)CCN1C(=O)OC(C)(C)C] | |
| 192 | 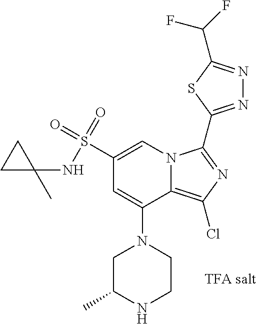 [SMILES: C[C@@H]1CN(c2cc(S(=O)(=O)NC3(C)CC3)cn3c(-c4nnc(C(F)F)s4)nc(Cl)c23)CCN1] | (R)-1-chloro-3-(5-(difluoromethyl)-1,3,4- thiadiazol-2-yl)-N-(1-methylcyclopropyl)-8-(3- methylpiperazin-1-yl)imidazo[1,5-a]pyridine-6- sulfonamide 2,2,2-trifluoroacetate |
| 193 | 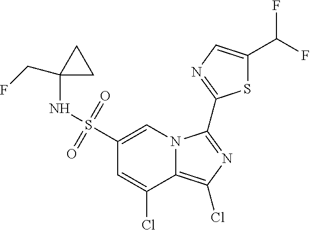 [SMILES: O=S(=O)(NC1(CF)CC1)c1cc(Cl)c2c(Cl)nc(-c3ncc(C(F)F)s3)n2c1] | 1,8-dichloro-3-(5-(difluoromethyl)thiazol-2-yl)-N- (1-(fluoromethyl)cyclopropyl)imidazo[1,5- a]pyridine-6-sulfonamide |
| 194 | 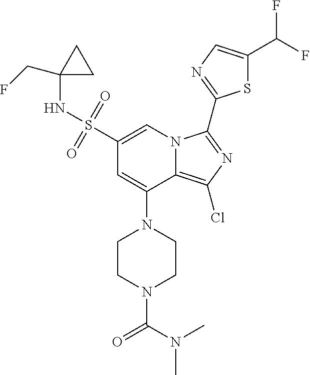 [SMILES: CN(C)C(=O)N1CCN(c2cc(S(=O)(=O)NC3(CF)CC3)cn3c(-c4ncc(C(F)F)s4)nc(Cl)c23)CC1] | 4-(1-chloro-3-(5-(difluoromethyl)thiazol-2-yl)-6- (N-(1- (fluoromethyl)cyclopropyl)sulfamoyl)imidazo[1,5- a]pyridin-8-yl)-N,N-dimethylpiperazine-1- carboxamide |
| 195 | 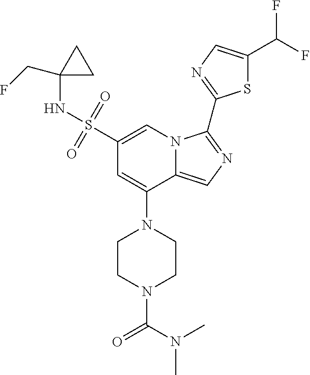 [SMILES: CN(C)C(=O)N1CCN(c2cc(S(=O)(=O)NC3(CF)CC3)cn3c(-c4ncc(C(F)F)s4)ncc23)CC1] | 4-(3-(5-(difluoromethyl)thiazol-2-yl)-6-(N-(1- (fluoromethyl)cyclopropyl)sulfamoyl)imidazo[1,5- a]pyridin-8-yl)-N,N-dimethylpiperazine-1- carboxamide |
| 196 | 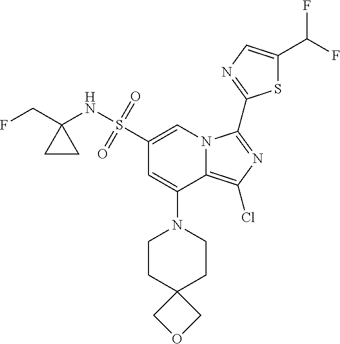 [SMILES: O=S(=O)(NC1(CF)CC1)c1cc(N2CCC3(CC2)COC3)c2c(Cl)nc(-c3ncc(C(F)F)s3)n2c1] | 1-chloro-3-(5-(difluoromethyl)thiazol-2-yl)-N-(1- (fluoromethyl)cyclopropyl)-8-(2-oxa-7- azaspiro[3.5]nonan-7-yl)imidazo[1,5-a]pyridine- 6-sulfonamide |
| 197a | 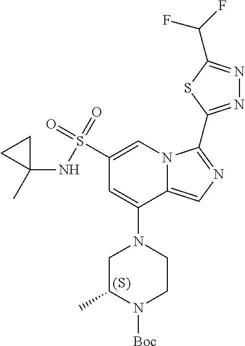 | (R)-tert-butyl 4-(3-(5-(difluoromethyl)-1,3,4- thiadiazol-2-yl)-6-(N-(1- methylcyclopropyl)sulfamoyl)imidazo[1,5- a]pyridin-8-yl)-2-methylpiperazine-1-carboxylate |
| 197 | 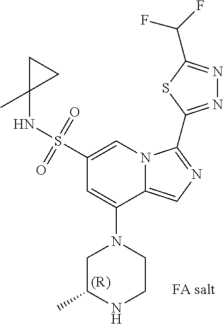 | (R)-3-(5-(difluoromethyl)-1,3,4-thiadiazol-2-yl)-N- (1-methylcyclopropyl)-8-(3-methylpiperazin-1- yl)imidazo[1,5-a]pyridine-6-sulfonamide formate |
| 198 | 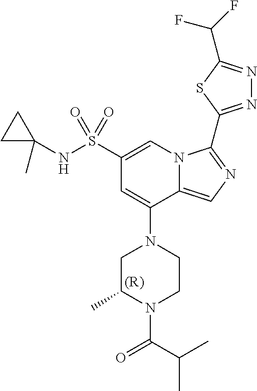 | (R)-3-(5-(difluoromethyl)-1,3,4-thiadiazol-2-yl)-8- (4-isobutyryl-3-methylpiperazin-1-yl)-N-(1- methylcyclopropyl)imidazo[1,5-a]pyridine-6- sulfonamide |
| 199 | 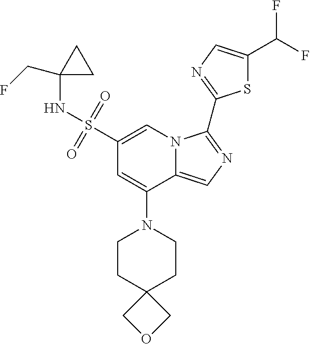 [SMILES: O=S(=O)(NC1(CF)CC1)c1cc(N2CCC3(CC2)COC3)c2cnc(-c3ncc(C(F)F)s3)n2c1] | 3-(5-(difluoromethyl)-1,3,4-thiadiazol-2-yl)-N-(1- (fluoromethyl)cyclopropyl)-8-(2-oxa-7- azaspiro[3.5]nonan-7-yl)imidazo[1,5-a]pyridine- 6-sulfonamide |
| 200 | 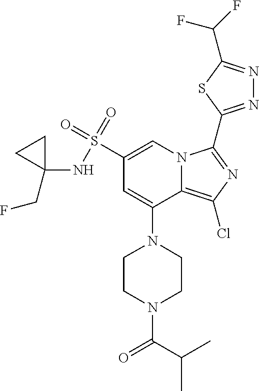 [SMILES: CC(C)C(=O)N1CCN(c2cc(S(=O)(=O)NC3(CF)CC3)cn3c(-c4nnc(C(F)F)s4)nc(Cl)c23)CC1] | 1-chloro-3-(5-(difluoromethyl)-1,3,4-thiadiazol-2- yl)-N-(1-(fluoromethyl)cyclopropyl)-8-(4- isobutyrylpiperazin-1-yl)imidazo[1,5-a]pyridine-6- sulfonamide |
| 201 | 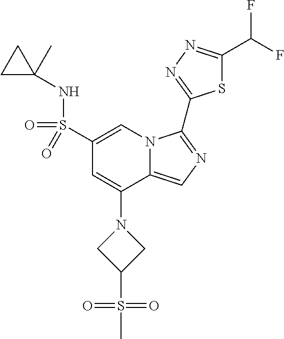 [SMILES: CC1(NS(=O)(=O)c2cc(N3CC(S(C)(=O)=O)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(3-(methylsulfonyl)azetidin- 1-yl)imidazo[1,2-a]pyridine-6-sulfonamide |
| 202 | 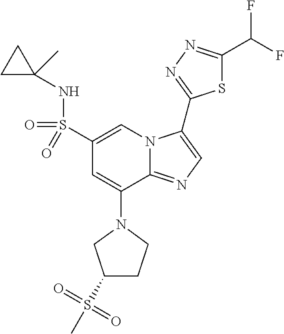 [SMILES: CC1(NS(=O)(=O)c2cc(N3CC[C@H](S(C)(=O)=O)C3)c3ncc(-c4nnc(C(F)F)s4)n3c2)CC1] | (S)-3-(5-(difluoromethyl)-1,3,4-thiadiazol-2-yl)-N- (1-methylcyclopropyl)-8-(3- (methylsulfonyl)pyrrolidin-1-yl)imidazo[1,2- a]pyridine-6-sulfonamide |
| 203 | 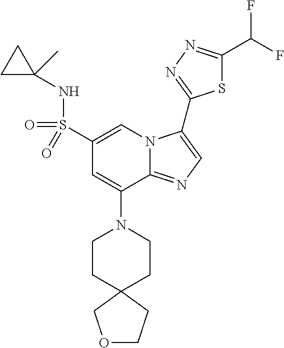 [SMILES: CC1(NS(=O)(=O)c2cc(N3CCC4(CCOC4)CC3)c3ncc(-c4nnc(C(F)F)s4)n3c2)CC1] | 3-(5-(difluoromethyl)-1,3,4-thiadiazol-2-yl)-N-(1- methylcyclopropyl)-8-(2-oxa-8- azaspiro[4.5]decan-8-yl)imidazo[1,2-a]pyridine-6- sulfonamide |
| 204 | 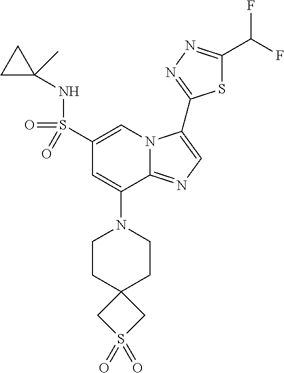 [SMILES: CC1(NS(=O)(=O)c2cc(N3CCC4(CC3)CS(=O)(=O)C4)c3ncc(-c4nnc(C(F)F)s4)n3c2)CC1] | 3-(5-(difluoromethyl)-1,3,4-thiadiazol-2-yl)-8-(2,2- dioxido-2-thia-7-azaspiro[3.5]nonan-7-yl)-N-(1- methylcyclopropyl)imidazo[1,2-a]pyridine-6- sulfonamide |
| 205 | 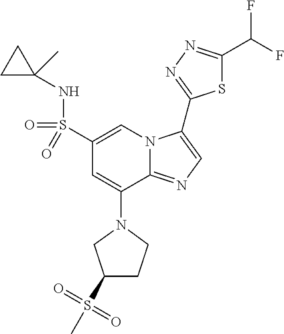 [SMILES: CC1(NS(=O)(=O)c2cc(N3CC[C@@H](S(C)(=O)=O)C3)c3ncc(-c4nnc(C(F)F)s4)n3c2)CC1] | (R)-3-(5-(difluoromethyl)-1,3,4-thiadiazol-2-yl)-N- (1-methylcyclopropyl)-8-(3- (methylsulfonyl)pyrrolidin-1-yl)imidazo[1,2- a]pyridine-6-sulfonamide |
| 206 | 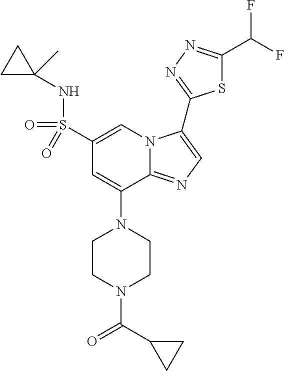 [SMILES: CC1(NS(=O)(=O)c2cc(N3CCN(C(=O)C4CC4)CC3)c3ncc(-c4nnc(C(F)F)s4)n3c2)CC1] | 8-(4-(cyclopropanecarbonyl)piperazin-1-yl)-3-(5- (difluoromethyl)-1,3,4-thiadiazol-2-yl)-N-(1- methylcyclopropyl)imidazo[1,2-a]pyridine-6- sulfonamide |
| 207 | 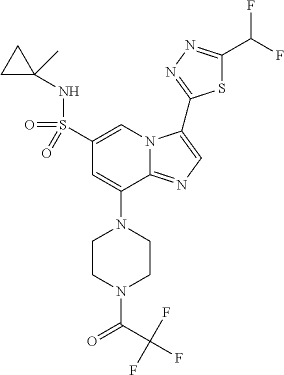 [SMILES: CC1(NS(=O)(=O)c2cc(N3CCN(C(=O)C(F)(F)F)CC3)c3ncc(-c4nnc(C(F)F)s4)n3c2)CC1] | 3-(5-(difluoromethyl)-1,3,4-thiadiazol-2-yl)-N-(1- methylcyclopropyl)-8-(4-(2,2,2- trifluoroacetyl)piperazin-1-yl)imidazo[1,2- a]pyridine-6-sulfonamide |
| 208 | 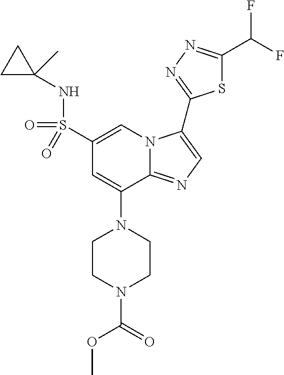 [SMILES: COC(=O)N1CCN(c2cc(S(=O)(=O)NC3(C)CC3)cn3c(-c4nnc(C(F)F)s4)cnc23)CC1] | methyl 4-(3-(5-(difluoromethyl)-1,3,4-thiadiazol-2- yl)-6-(N-(1- methylcyclopropyl)sulfamoyl)imidazo[1,2- a]pyridin-8-yl)piperazine-1-carboxylate |
| 209 | 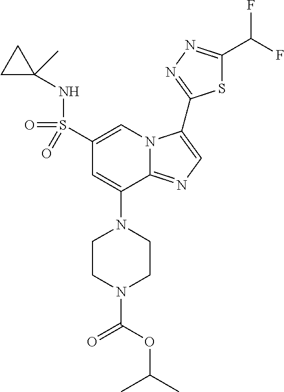 [SMILES: CC(C)OC(=O)N1CCN(c2cc(S(=O)(=O)NC3(C)CC3)cn3c(-c4nnc(C(F)F)s4)cnc23)CC1] | isopropyl 4-(3-(5-(difluoromethyl)-1,3,4- thiadiazol-2-yl)-6-(N-(1- methylcyclopropyl)sulfamoyl)imidazo[1,2- a]pyridin-8-yl)piperazine-1-carboxylate |
| 210 | 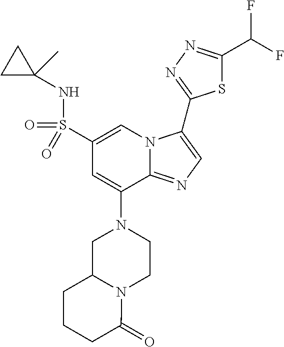 [SMILES: CC1(NS(=O)(=O)c2cc(N3CCN4C(=O)CCCC4C3)c3ncc(-c4nnc(C(F)F)s4)n3c2)CC1] | 3-(5-(difluoromethyl)-1,3,4-thiadiazol-2-yl)-N-(1- methylcyclopropyl)-8-(6-oxooctahydro-2H- pyrido[1,2-a]pyrazin-2-yl)imidazo[1,2-a]pyridine- 6-sulfonamide |
| 211 | 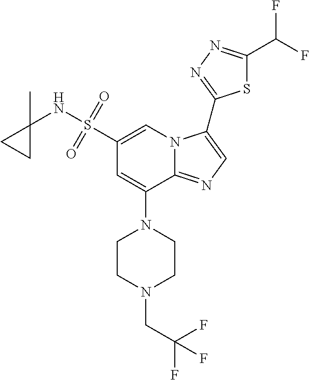 [SMILES: CC1(NS(=O)(=O)c2cc(N3CCN(CC(F)(F)F)CC3)c3ncc(-c4nnc(C(F)F)s4)n3c2)CC1] | 3-(5-(difluoromethyl)-1,3,4-thiadiazol-2-yl)-N-(1- methylcyclopropyl)-8-(4-(2,2,2- trifluoroethyl)piperazin-1-yl)imidazo[1,2- a]pyridine-6-sulfonamide |
| 212 | 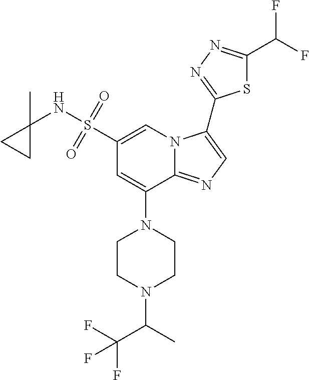 [SMILES: CC(N1CCN(c2cc(S(=O)(=O)NC3(C)CC3)cn3c(-c4nnc(C(F)F)s4)cnc23)CC1)C(F)(F)F] | 3-(5-(difluoromethyl)-1,3,4-thiadiazol-2-yl)-N-(1- methylcyclopropyl)-8-(4-(1,1,1-trifluoropropan-2- yl)piperazin-1-yl)imidazo[1,2-a]pyridine-6- sulfonamide |
| 213 | 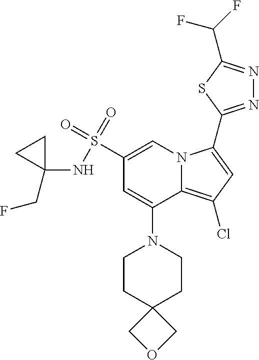 [SMILES: O=S(=O)(NC1(CF)CC1)c1cc(N2CCC3(CC2)COC3)c2c(Cl)cc(-c3nnc(C(F)F)s3)n2c1] | 1-chloro-3-(5-(difluoromethyl)-1,3,4-thiadiazol-2- yl)-N-(1-(fluoromethyl)cyclopropyl)-8-(2-oxa-7- azaspiro[3.5]nonan-7-yl)indolizine-6-sulfonamide |
| 214 | 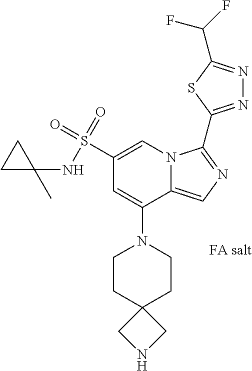 [SMILES: CC1(NS(=O)(=O)c2cc(N3CCC4(CC3)CNC4)c3cnc(-c4nnc(C(F)F)s4)n3c2)CC1] | 3-(5-(difluoromethyl)-1,3,4-thiadiazol-2-yl)-N-(1- methylcyclopropyl)-8-(2,7-diazaspiro[3.5]nonan- 7-yl)imidazo[1,5-a]pyridine-6-sulfonamide formate |
| 215 | 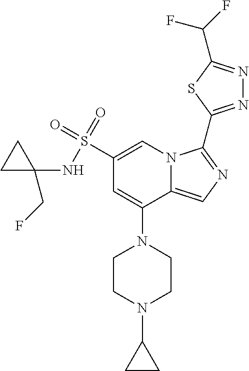 [SMILES: O=S(=O)(NC1(CF)CC1)c1cc(N2CCN(C3CC3)CC2)c2cnc(-c3nnc(C(F)F)s3)n2c1] | 8-(4-cyclopropylpiperazin-1-yl)-3-(5- (difluoromethyl)-1,3,4-thiadiazol-2-yl)-N-(1- (fluoromethyl)cyclopropyl)imidazo[1,5-a]pyridine- 6-sulfonamide |
| 216 | 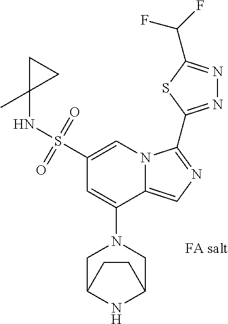 [SMILES: CC1(NS(=O)(=O)c2cc(N3CC4CCC(C3)N4)c3cnc(-c4nnc(C(F)F)s4)n3c2)CC1] | 8-(3,8-diazabicyclo[3.2.1]octan-3-yl)-3-(5- (difluoromethyl)-1,3,4-thiadiazol-2-yl)-N-(1- methylcyclopropyl)imidazo[1,5-a]pyridine-6- sulfonamide formate |
| 217 | 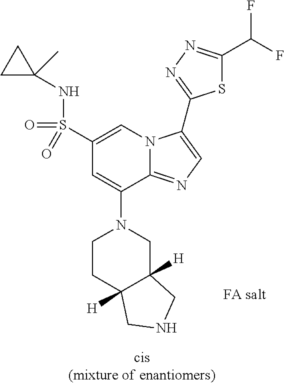 [SMILES: CC1(NS(=O)(=O)c2cc(N3CC[C@H]4CNC[C@H]4C3)c3ncc(-c4nnc(C(F)F)s4)n3c2)CC1] | 1-chloro-3-(5-(difluoromethyl)-1,3,4-thiadiazol-2- yl)-N-(1-methylcyclopropyl)-8-(cis)-octahydro-5H- pyrrolo[3,4-c]pyridin-5-yl)imidazo[1,5-a]pyridine- 6-sulfonamide formate |
| 218 | 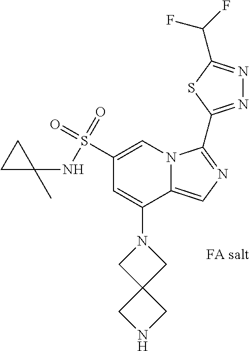 [SMILES: CC1(NS(=O)(=O)c2cc(N3CC4(CNC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(2,6-diazaspiro[3.3]heptan- 2-yl)imidazo[1,5-a]pyridine-6-sulfonamide formate |
| 219 | 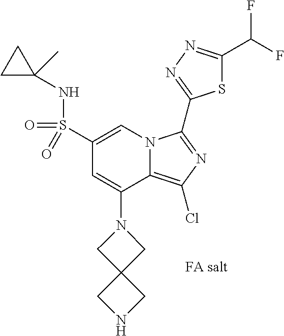 [SMILES: CC1(NS(=O)(=O)c2cc(N3CC4(CNC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2,6- diazaspiro[3.3]heptan-2-yl)imidazo[1,5- a]pyridine-6-sulfonamide formate |
| 220 | 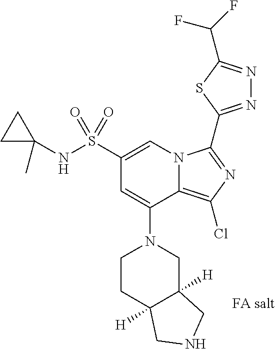 [SMILES: CC1(NS(=O)(=O)c2cc(N3CC[C@@H]4CNC[C@@H]4C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-((3aR,7aS)- octahydro-5H-pyrrolo[3,4-c]pyridin-5- yl)imidazo[1,5-a]pyridine-6-sulfonamide formate |
| 221 | 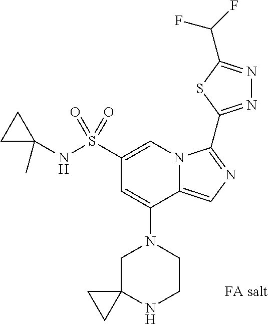 [SMILES: CC1(NS(=O)(=O)c2cc(N3CCNC4(CC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(4,7-diazaspiro[2.5]octan-7- yl)imidazo[1,5-a]pyridine-6-sulfonamide formate |
| 222 | 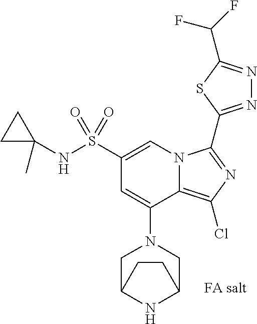 [SMILES: CC1(NS(=O)(=O)c2cc(N3CC4CCC(C3)N4)c3c(Cl)nc(-c4nnc(C(F)F)s4)n3c2)CC1] | 8-((1R,5S)-3,8-diazabicyclo[3.2.1]octan-3-yl)-1- chloro-3-(5-(difluoromethyl)-1,3,4-thiadiazol-2- yl)-N-(1-methylcyclopropyl)imidazo[1,5- a]pyridine-6-sulfonamide formate |
| 223 | 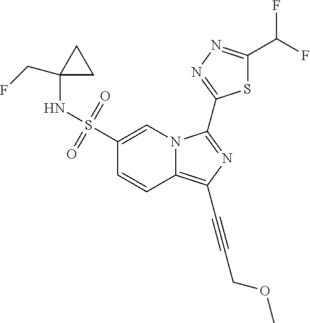 [SMILES: COCC#Cc1nc(-c2nnc(C(F)F)s2)n2cc(S(=O)(=O)NC3(CF)CC3)ccc12] | 3-(5-(difluoromethyl)-1,3,4-thiadiazol-2-yl)-N-(1- (fluoromethyl)cyclopropyl)-1-(3-methoxyprop-1- yn-1-yl)imidazo[1,5-a]pyridine-6-sulfonamide |
| 224 | 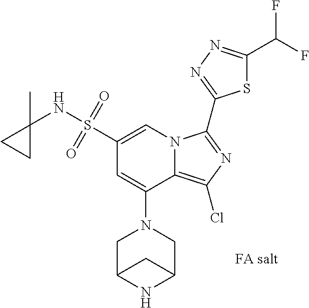 [SMILES: CC1(NS(=O)(=O)c2cc(N3CC4CC(C3)N4)c3c(Cl)nc(-c4nnc(C(F)F)s4)n3c2)CC1] | 8-(3,6-diazabicyclo[3.1.1]heptan-3-yl)-1-chloro-3- (5-(difluoromethyl)-1,3,4-thiadiazol-2-yl)-N-(1- methylcyclopropyl)imidazo[1,5-a]pyridine-6- sulfonamide formate |
| 225 | 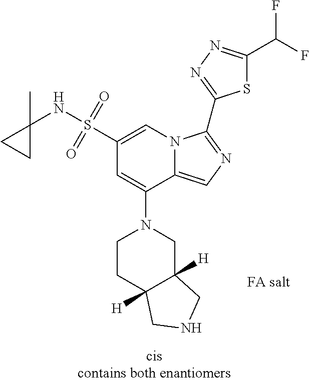 [SMILES: CC1(NS(=O)(=O)c2cc(N3CC[C@H]4CNC[C@H]4C3)c3cnc(-c4nnc(C(F)F)s4)n3c2)CC1] | 3-(5-(difluoromethyl)-1,3,4-thiadiazol-2-yl)-N-(1- methylcyclopropyl)-8-(cis)-octahydro-5H- pyrrolo[3,4-c]pyridin-5-yl)imidazo[1,5-a]pyridine- 6-sulfonamide formate |
| 226 | 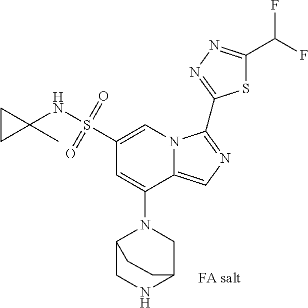 [SMILES: CC1(NS(=O)(=O)c2cc(N3CC4CCC3CN4)c3cnc(-c4nnc(C(F)F)s4)n3c2)CC1] | 8-(2,5-diazabicyclo[2.2.2]octan-2-yl)-3-(5- (difluoromethyl)-1,3,4-thiadiazol-2-yl)-N-(1- methylcyclopropyl)imidazo[1,5-a]pyridine-6- sulfonamide formate |
| 227 | 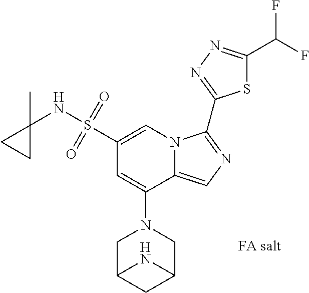 [SMILES: CC1(NS(=O)(=O)c2cc(N3CC4CC(C3)N4)c3cnc(-c4nnc(C(F)F)s4)n3c2)CC1] | 8-(3,6-diazabicyclo[3.1.1]heptan-3-yl)-3-(5- (difluoromethyl)-1,3,4-thiadiazol-2-yl)-N-(1- methylcyclopropyl)imidazo[1,5-a]pyridine-6- sulfonamide formate |
| 228 | 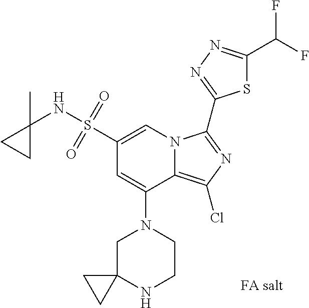 [SMILES: CC1(NS(=O)(=O)c2cc(N3CCNC4(CC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(4,7- diazaspiro[2.5]octan-7-yl)imidazo[1,5-a]pyridine- 6-sulfonamide formate |
| 229 | 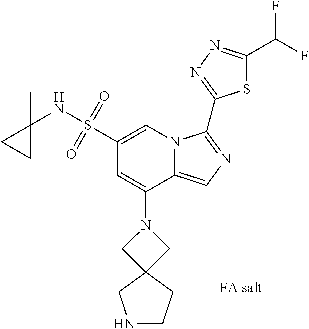 [SMILES: CC1(NS(=O)(=O)c2cc(N3CC4(CCNC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(2,6-diazaspiro[3.4]octan-2- yl)imidazo[1,5-a]pyridine-6-sulfonamide formate |
| 230 | 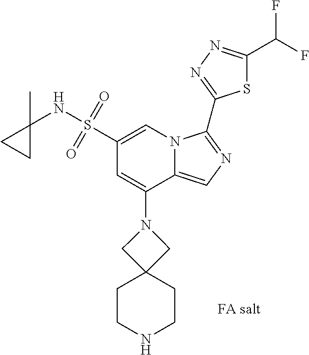 [SMILES: CC1(NS(=O)(=O)c2cc(N3CC4(CCNCC4)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(2,7-diazaspiro[3.5]nonan- 2-yl)imidazo[1,5-a]pyridine-6-sulfonamide formate |
| 231 | 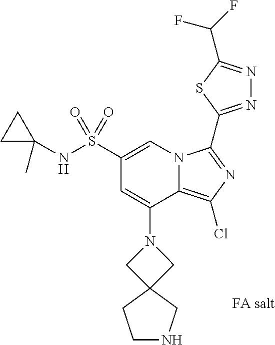 [SMILES: CC1(NS(=O)(=O)c2cc(N3CC4(CCNC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2,6- diazaspiro[3.4]octan-2-yl)imidazo[1,5-a]pyridine- 6-sulfonamide formate |
| 232 | 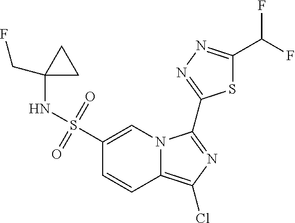 [SMILES: O=S(=O)(NC1(CF)CC1)c1ccc2c(Cl)nc(-c3nnc(C(F)F)s3)n2c1] | 1-chloro-3-(5-(difluoromethyl)-1,3,4-thiadiazol-2- yl)-N-(1-(fluoromethyl)cyclopropyl)imidazo[1,5- a]pyridine-6-sulfonamide |
| 233 | 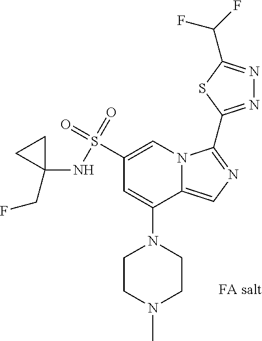 [SMILES: CN1CCN(c2cc(S(=O)(=O)NC3(CF)CC3)cn3c(-c4nnc(C(F)F)s4)ncc23)CC1] | 3-(5-(difluoromethyl)-1,3,4-thiadiazol-2-yl)-N-(1- (fluoromethyl)cyclopropyl)-8-(4-methylpiperazin- 1-yl)imidazo[1,5-a]pyridine-6-sulfonamide formate |
| 234 | 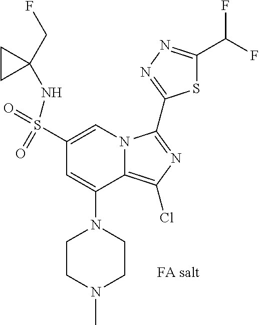 [SMILES: CN1CCN(c2cc(S(=O)(=O)NC3(CF)CC3)cn3c(-c4nnc(C(F)F)s4)nc(Cl)c23)CC1] | 1-chloro-3-(5-(difluoromethyl)-1,3,4-thiadiazol-2- yl)-N-(1-(fluoromethyl)cyclopropyl)-8-(4- methylpiperazin-1-yl)imidazo[1,5-a]pyridine-6- sulfonamide formate |
| 235 | 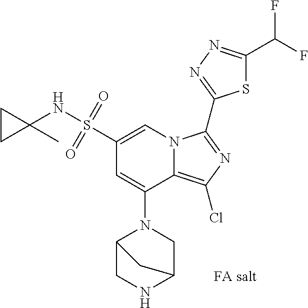 [SMILES: CC1(NS(=O)(=O)c2cc(N3CC4CC3CN4)c3c(Cl)nc(-c4nnc(C(F)F)s4)n3c2)CC1] | 8-(2,5-diazabicyclo[2.2.1]heptan-2-yl)-1-chloro-3- (5-(difluoromethyl)-1,3,4-thiadiazol-2-yl)-N-(1- methylcyclopropyl)imidazo[1,5-a]pyridine-6- sulfonamide formate |
| 236 | 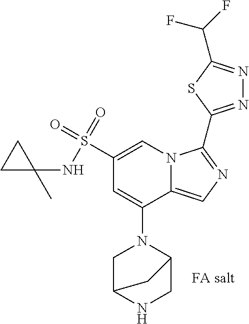 [SMILES: CC1(NS(=O)(=O)c2cc(N3CC4CC3CN4)c3cnc(-c4nnc(C(F)F)s4)n3c2)CC1] | 8-(2,5-diazabicyclo[2.2.1]heptan-2-yl)-3-(5- (difluoromethyl)-1,3,4-thiadiazol-2-yl)-N-(1- methylcyclopropyl)imidazo[1,5-a]pyridine-6- sulfonamide formate |
| 237 | 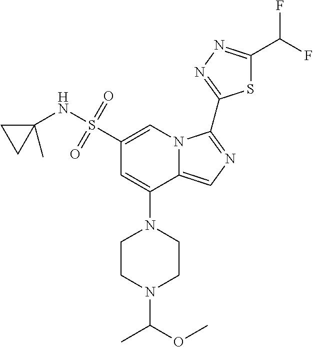 [SMILES: COC(C)N1CCN(c2cc(S(=O)(=O)NC3(C)CC3)cn3c(-c4nnc(C(F)F)s4)ncc23)CC1] | 3-(5-(difluoromethyl)-1,3,4-thiadiazol-2-yl)-8-(4- (1-methoxyethyl)piperidin-1-yl)-N-(1- methylcyclopropyl)imidazo[1,5-a]pyridine-6- sulfonamide |
| 238 | 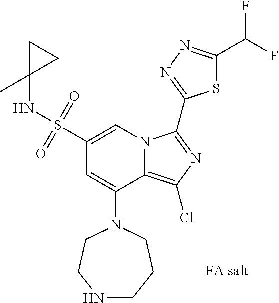 [SMILES: CC1(NS(=O)(=O)c2cc(N3CCCNCC3)c3c(Cl)nc(-c4nnc(C(F)F)s4)n3c2)CC1] | 1-chloro-8-(1,4-diazepan-1-yl)-3-(5- (difluoromethyl)-1,3,4-thiadiazol-2-yl)-N-(1- methylcyclopropyl)imidazo[1,5-a]pyridine-6- sulfonamide formate |
| 239 | 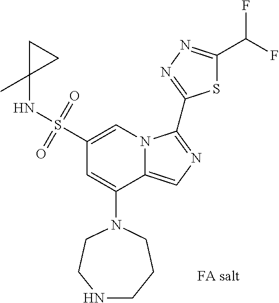 [SMILES: CC1(NS(=O)(=O)c2cc(N3CCCNCC3)c3cnc(-c4nnc(C(F)F)s4)n3c2)CC1] | 8-(1,4-diazepan-1-yl)-3-(5-(difluoromethyl)-1,3,4- thiadiazol-2-yl)-N-(1- methylcyclopropyl)imidazo[1,5-a]pyridine-6- sulfonamide formate |
| 240 | 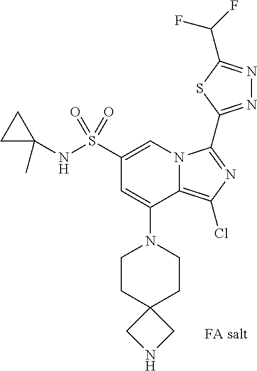 [SMILES: CC1(NS(=O)(=O)c2cc(N3CCC4(CC3)CNC4)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2,7- diazaspiro[3.5]nonan-7-yl)imidazo[1,5-a]pyridine- 6-sulfonamide formate |
| 241 | 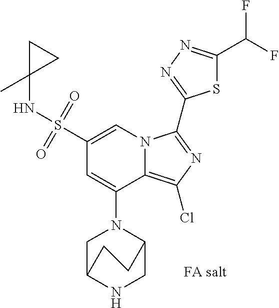 [SMILES: CC1(NS(=O)(=O)c2cc(N3CC4CCC3CN4)c3c(Cl)nc(-c4nnc(C(F)F)s4)n3c2)CC1] | 8-(2,5-diazabicyclo[2.2.2]octan-2-yl)-1-chloro-3- (5-(difluoromethyl)-1,3,4-thiadiazol-2-yl)-N-(1- methylcyclopropyl)imidazo[1,5-a]pyridine-6- sulfonamide formate |
| 242 | 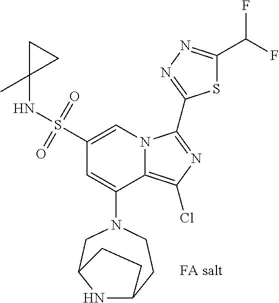 [SMILES: CC1(NS(=O)(=O)c2cc(N3CCC4CCC(C3)N4)c3c(Cl)nc(-c4nnc(C(F)F)s4)n3c2)CC1] | 8-(3,9-diazabicyclo[4.2.1]nonan-3-yl)-1-chloro-3- (5-(difluoromethyl)-1,3,4-thiadiazol-2-yl)-N-(1- methylcyclopropyl)imidazo[1,5-a]pyridine-6- sulfonamide formate |
| 243 | 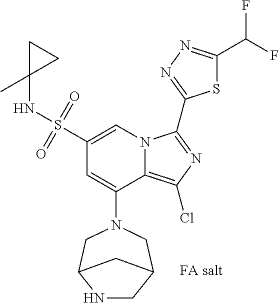 [SMILES: CC1(NS(=O)(=O)c2cc(N3CC4CNC(C4)C3)c3c(Cl)nc(-c4nnc(C(F)F)s4)n3c2)CC1] | 8-(3,6-diazabicyclo[3.2.1]octan-3-yl)-1-chloro-3- (5-(difluoromethyl)-1,3,4-thiadiazol-2-yl)-N-(1- methylcyclopropyl)imidazo[1,5-a]pyridine-6- sulfonamide formate |
| 244 | 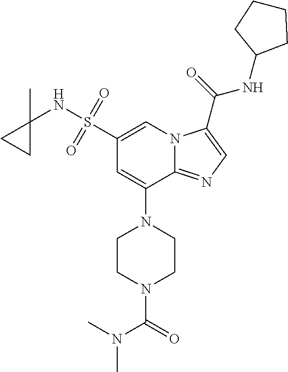 [SMILES: CN(C)C(=O)N1CCN(c2cc(S(=O)(=O)NC3(C)CC3)cn3c(C(=O)NC4CCCC4)cnc23)CC1] | N-cyclopentyl-8-(4-(dimethylcarbamoyl)piperazin- 1-yl)-6-(N-(1- methylcyclopropyl)sulfamoyl)imidazo[1,2- a]pyridine-3-carboxamide |
| 245 | 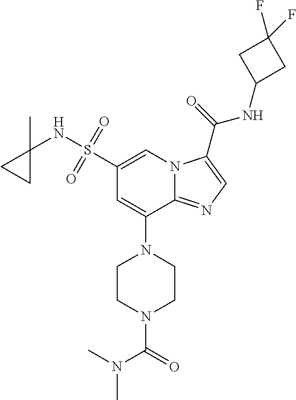 [SMILES: CN(C)C(=O)N1CCN(c2cc(S(=O)(=O)NC3(C)CC3)cn3c(C(=O)NC4CC(F)(F)C4)cnc23)CC1] | N-(3,3-difluorocyclobutyl)-8-(4- (dimethylcarbamoyl)piperazin-1-yl)-6-(N-(1- methylcyclopropyl)sulfamoyl)imidazo[1,2- a]pyridine-3-carboxamide |
| 246 | 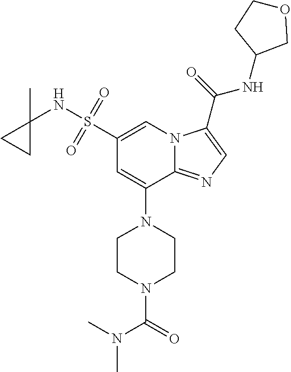 [SMILES: CN(C)C(=O)N1CCN(c2cc(S(=O)(=O)NC3(C)CC3)cn3c(C(=O)NC4CCOC4)cnc23)CC1] | 8-(4-(dimethylcarbamoyl)piperazin-1-yl)-6-(N-(1- methylcyclopropyl)sulfamoyl)-N-(tetrahydrofuran- 3-yl)imidazo[1,2-a]pyridine-3-carboxamide |
| 247 | 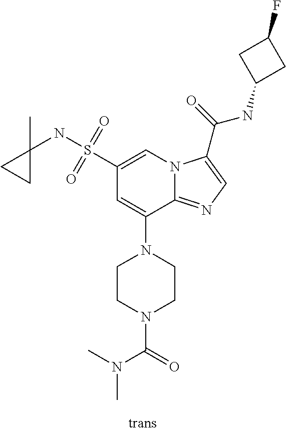 [SMILES: CN(C)C(=O)N1CCN(c2cc(S(=O)(=O)NC3(C)CC3)cn3c(C(=O)N[C@H]4C[C@H](F)C4)cnc23)CC1] | 8-(4-(dimethylcarbamoyl)piperazin-1-yl)-N- ((1r,3r)-3-fluorocyclobutyl)-6-(N-(1- methylcyclopropyl)sulfamoyl)imidazo[1,2- a]pyridine-3-carboxamide |
| 248 | 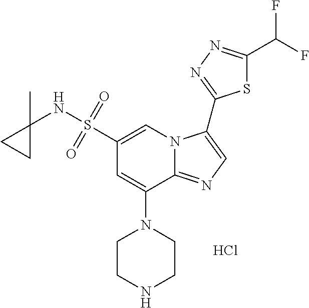 [SMILES: CC1(NS(=O)(=O)c2cc(N3CCNCC3)c3ncc(-c4nnc(C(F)F)s4)n3c2)CC1 ; Cl] | 3-(5-(difluoromethyl)-1,3,4-thiadiazol-2-yl)-N-(1- methylcyclopropyl)-8-(piperazin-1-yl)imidazo[1,2- a]pyridine-6-sulfonamide hydrochloride |
| 249 | 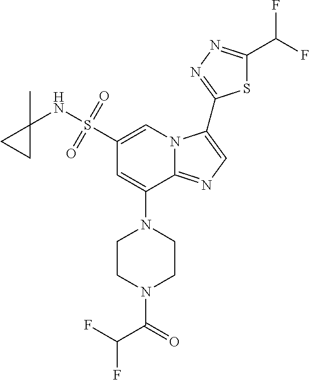 [SMILES: CC1(NS(=O)(=O)c2cc(N3CCN(C(=O)C(F)F)CC3)c3ncc(-c4nnc(C(F)F)s4)n3c2)CC1] | 8-(4-(2,2-difluoroacetyl)piperazin-1-yl)-3-(5- (difluoromethyl)-1,3,4-thiadiazol-2-yl)-N-(1- methylcyclopropyl)imidazo[1,2-a]pyridine-6- sulfonamide |
| 250 | 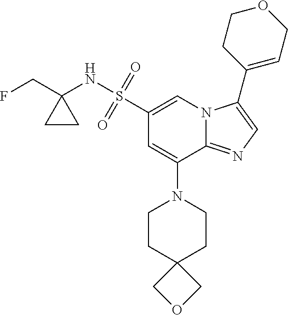 [SMILES: O=S(=O)(NC1(CF)CC1)c1cc(N2CCC3(CC2)COC3)c2ncc(C3=CCOCC3)n2c1] | 3-(3,6-dihydro-2H-pyran-4-yl)-N-(1- (fluoromethyl)cyclopropyl)-8-(2-oxa-7- azaspiro[3.5]nonan-7-yl)imidazo[1,2-a]pyridine- 6-sulfonamide |
| 251 | 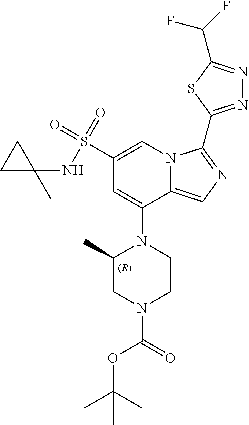 | tert-butyl (R)-4-(3-(5-(difluoromethyl)-1,3,4- thiadiazol-2-yl)-6-(N-(1- methylcyclopropyl)sulfamoyl)imidazo[1,5- a]pyridin-8-yl)-3-methylpiperazine-1-carboxylate |
| 252 | 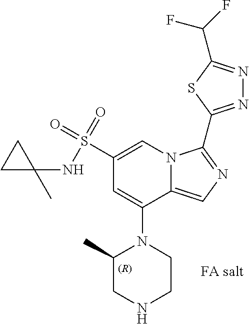 | (R)-3-(5-(difluoromethyl)-1,3,4-thiadiazol-2-yl)-N- (1-methylcyclopropyl)-8-(2-methylpiperazin-1- yl)imidazo[1,5-a]pyridine-6-sulfonamide formate |
| 253 | 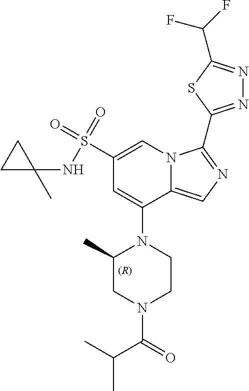 | (R)-3-(5-(difluoromethyl)-1,3,4-thiadiazol-2-yl)-8- (4-isobutyryl-2-methylpiperazin-1-yl)-N-(1- methylcyclopropyl)imidazo[1,5-a]pyridine-6- sulfonamide |
| 255 | 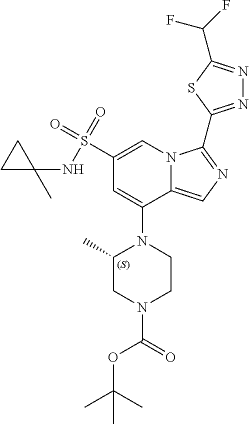 | tert-butyl (S)-4-(3-(5-(difluoromethyl)-1,3,4- thiadiazol-2-yl)-6-(N-(1- methylcyclopropyl)sulfamoyl)imidazo[1,5- a]pyridin-8-yl)-3-methylpiperazine-1-carboxylate |
| 256 | 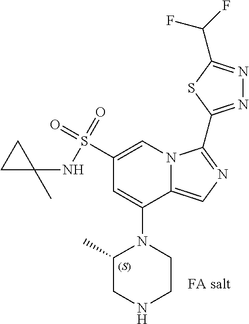 | (S)-3-(5-(difluoromethyl)-1,3,4-thiadiazol-2-yl)-N- (1-methylcyclopropyl)-8-(2-methylpiperazin-1- yl)imidazo[1,5-a]pyridine-6-sulfonamide formate |
| 257 | 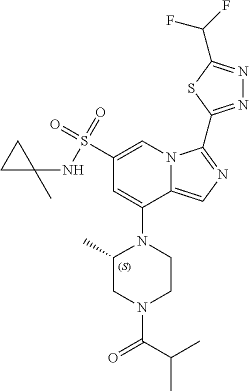 | (S)-3-(5-(difluoromethyl)-1,3,4-thiadiazol-2-yl)-8- (4-isobutyryl-2-methylpiperazin-1-yl)-N-(1- methylcyclopropyl)imidazo[1,5-a]pyridine-6- sulfonamide |
| 258 | 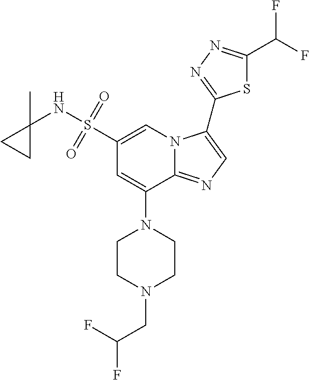 [SMILES: CC1(NS(=O)(=O)c2cc(N3CCN(CC(F)F)CC3)c3ncc(-c4nnc(C(F)F)s4)n3c2)CC1] | 8-(4-(2,2-difluoroethyl)piperazin-1-yl)-3-(5- (difluoromethyl)-1,3,4-thiadiazol-2-yl)-N-(1- methylcyclopropyl)imidazo[1,2-a]pyridine-6- sulfonamide |
| 259 | 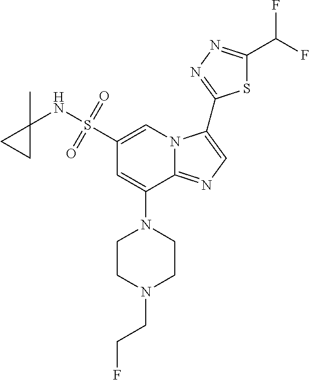 [SMILES: CC1(NS(=O)(=O)c2cc(N3CCN(CCF)CC3)c3ncc(-c4nnc(C(F)F)s4)n3c2)CC1] | 3-(5-(difluoromethyl)-1,3,4-thiadiazol-2-yl)-8-(4- (2-fluoroethyl)piperazin-1-yl)-N-(1- methylcyclopropyl)imidazo[1,2-a]pyridine-6- sulfonamide |
| 260 | 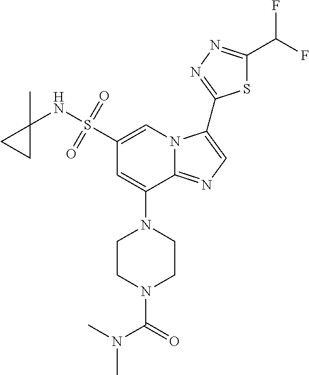 [SMILES: CN(C)C(=O)N1CCN(c2cc(S(=O)(=O)NC3(C)CC3)cn3c(-c4nnc(C(F)F)s4)cnc23)CC1] | 4-(3-(5-(difluoromethyl)pyrazin-2-yl)-6-(N-(1- methylcyclopropyl)sulfamoyl)imidazo[1,2- a]pyridin-8-yl)-N,N-dimethylpiperazine-1- carboxamide |
| 261 | 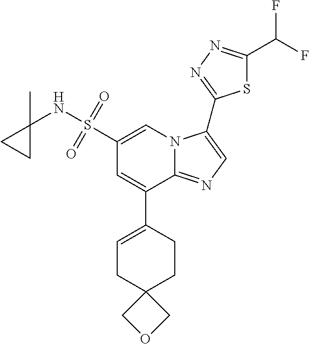 [SMILES: CC1(NS(=O)(=O)c2cc(C3=CCC4(CC3)COC4)c3ncc(-c4nnc(C(F)F)s4)n3c2)CC1] | 3-(5-(difluoromethyl)-1,3,4-thiadiazol-2-yl)-N-(1- methylcyclopropyl)-8-(2-oxaspiro[3.5]non-6-en-7- yl)imidazo[1,2-a]pyridine-6-sulfonamide |
| 262 | 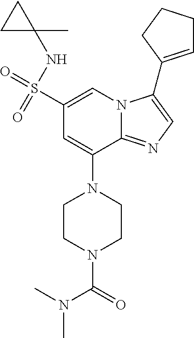 [SMILES: CN(C)C(=O)N1CCN(c2cc(S(=O)(=O)NC3(C)CC3)cn3c(C4=CCCC4)cnc23)CC1] | 4-(3-(cyclopent-1-en-1-yl)-6-(N-(1- methylcyclopropyl)sulfamoyl)imidazo[1,2- a]pyridin-8-yl)-N,N-dimethylpiperazine-1- carboxamide |
| 263 | 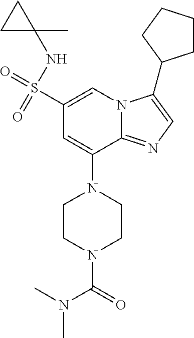 [SMILES: CN(C)C(=O)N1CCN(c2cc(S(=O)(=O)NC3(C)CC3)cn3c(C4CCCC4)cnc23)CC1] | 4-(3-cyclopentyl-6-(N-(1- methylcyclopropyl)sulfamoyl)imidazo[1,2- a]pyridin-8-yl)-N,N-dimethylpiperazine-1- carboxamide |
| 264 | 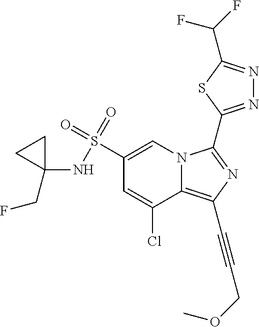 [SMILES: COCC#Cc1nc(-c2nnc(C(F)F)s2)n2cc(S(=O)(=O)NC3(CF)CC3)cc(Cl)c12] | 8-chloro-3-(5-(difluoromethyl)-1,3,4-thiadiazol-2- yl)-N-(1-(fluoromethyl)cyclopropyl)-1-(3- methoxyprop-1-yn-1-yl)indolizine-6-sulfonamide |
| 265 | 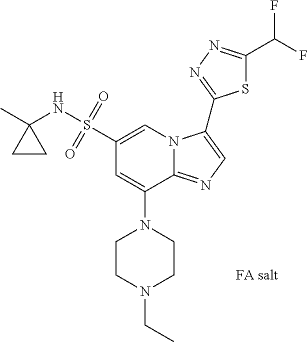 [SMILES: CCN1CCN(c2cc(S(=O)(=O)NC3(C)CC3)cn3c(-c4nnc(C(F)F)s4)cnc23)CC1 ; "FA salt"] | 3-(5-(difluoromethyl)-1,3,4-thiadiazol-2-yl)-8-(4- ethylpiperazin-1-yl)-N-(1- methylcyclopropyl)imidazo[1,2-a]pyridine-6- sulfonamide formate |
| 266 | 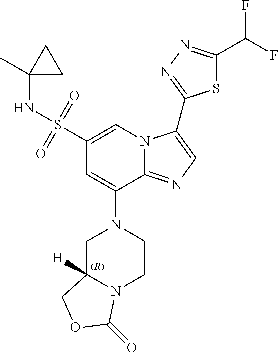 | (R)-3-(5-(difluoromethyl)-1,3,4-thiadiazol-2-yl)-N- (1-methylcyclopropyl)-8-(6- oxohexahydropyrrolo[1,2-a]pyrazin-2(1H)- yl)imidazo[1,2-a]pyridine-6-sulfonamide |
| 267 | 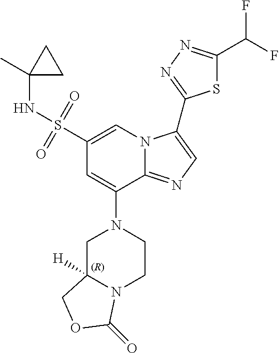 | (R)-3-(5-(difluoromethyl)-1,3,4-thiadiazol-2-yl)-N- (1-methylcyclopropyl)-8-(3-oxotetrahydro-3H- oxazolo[3,4-a]pyrazin-7(1H)-yl)imidazo[1,2- a]pyridine-6-sulfonamide |
| 268 | 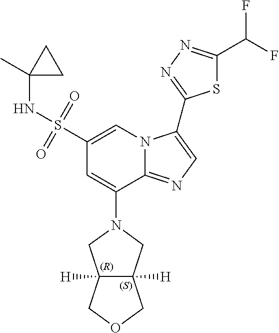 | 3-(5-(difluoromethyl)-1,3,4-thiadiazol-2-yl)-N-(1- methylcyclopropyl)-8-((3aR,6aS)-tetrahydro-1H- furo[3,4-c]pyrrol-5(3H)-yl)imidazo[1,2-a]pyridine- 6-sulfonamide |
| 269 | 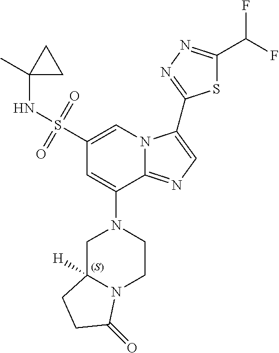 | (S)-3-(5-(difluoromethyl)-1,3,4-thiadiazol-2-yl)-N- (1-methylcyclopropyl)-8-(6- oxohexahydropyrrolo[1,2-a]pyrazin-2(1H)- yl)imidazo[1,2-a]pyridine-6-sulfonamide |
| 270 | 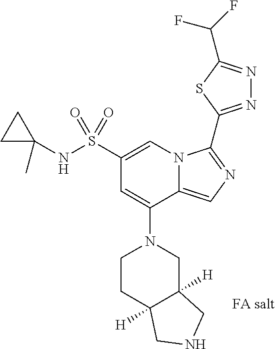 [SMILES: CC1(NS(=O)(=O)c2cc(N3CC[C@@H]4CNC[C@@H]4C3)c3cnc(-c4nnc(C(F)F)s4)n3c2)CC1] | 3-(5-(difluoromethyl)-1,3,4-thiadiazol-2-yl)-N-(1- methylcyclopropyl)-8-((3aR,7aS)-octahydro-5H- pyrrolo[3,4-c]pyridin-5-yl)imidazo[1,5-a]pyridine- 6-sulfonamide formate |
| 271 | 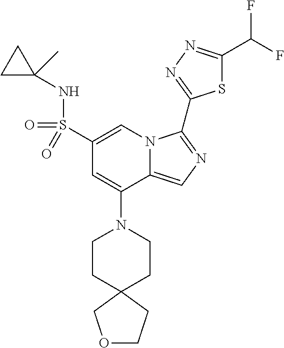 [SMILES: CC1(NS(=O)(=O)c2cc(N3CCC4(CCOC4)CC3)c3cnc(-c4nnc(C(F)F)s4)n3c2)CC1] | 3-(5-(difluoromethyl)-1,3,4-thiadiazol-2-yl)-N-(1- methylcyclopropyl)-8-(2-oxa-8- azaspiro[4.5]decan-8-yl)imidazo[1,5-a]pyridine-6- sulfonamide |
| 272 | 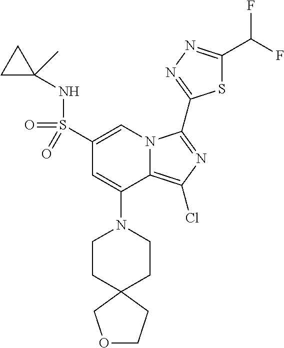 [SMILES: CC1(NS(=O)(=O)c2cc(N3CCC4(CCOC4)CC3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2-oxa-8- azaspiro[4.5]decan-8-yl)imidazo[1,5-a]pyridine-6- sulfonamide |
| 273 | 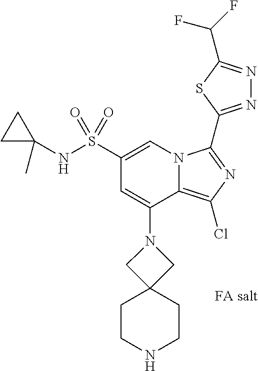 [SMILES: CC1(NS(=O)(=O)c2cc(N3CC4(CCNCC4)C3)c3c(Cl)nc(-c4nnc(C(F)F)s4)n3c2)CC1] | 1-chloro-3-(5-(difluoromethyl)-1,3,4-thiadiazol-2- yl)-N-(1-methylcyclopropyl)-8-(2,7- diazaspiro[3.5]nonan-2-yl)imidazo[1,5-a]pyridine- 6-sulfonamide formate |
| 274 | 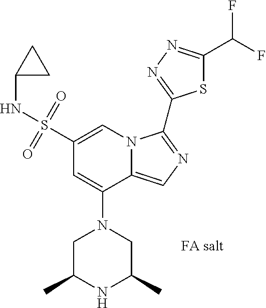 [SMILES: C[C@@H]1CN(c2cc(S(=O)(=O)NC3CC3)cn3c(-c4nnc(C(F)F)s4)ncc23)C[C@H](C)N1 ; "FA salt"] | 3-(5-(difluoromethyl)-1,3,4-thiadiazol-2-yl)-8- ((3S,5R)-3,5-dimethylpiperazin-1-yl)-N-(1- methylcyclopropyl)imidazo[1,5-a]pyridine-6- sulfonamide formate |
| 275 | 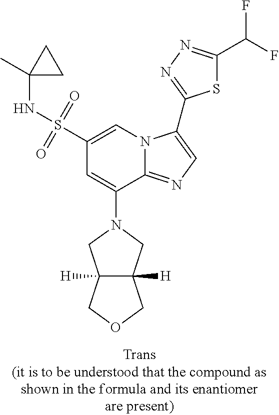 [SMILES: CC1(NS(=O)(=O)c2cc(N3C[C@@H]4COC[C@H]4C3)c3ncc(-c4nnc(C(F)F)s4)n3c2)CC1] | 3-(5-(difluoromethyl)-1,3,4-thiadiazol-2-yl)-N-(1- methylcyclopropyl)-8-((trans)-tetrahydro-1H- furo[3,4-c]pyrrol-5(3H)-yl)imidazo[1,2-a]pyridine- 6-sulfonamide |
| 276 | 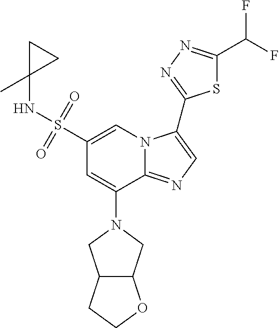 [SMILES: CC1(NS(=O)(=O)c2cc(N3CC4CCOC4C3)c3ncc(-c4nnc(C(F)F)s4)n3c2)CC1] | 3-(5-(difluoromethyl)-1,3,4-thiadiazol-2-yl)-8- (hexahydro-5H-furo[2,3-c]pyrrol-5-yl)-N-(1- methylcyclopropyl)imidazo[1,2-a]pyridine-6- sulfonamide |
| 277 | 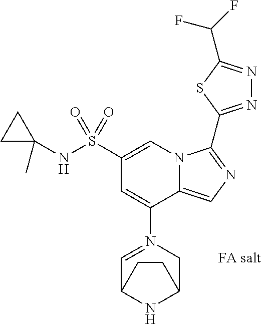 | 8-cis-8-azabicyclo[3.2.1]oct-2-en-3-yl)-3-(5- (difluoromethyl)-1,3,4-thiadiazol-2-yl)-N-(1- methylcyclopropyl)imidazo[1,5-a]pyridine-6- sulfonamide formate |
| 278 | 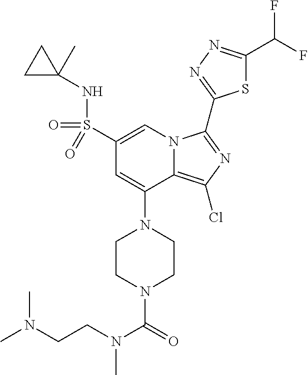 [SMILES: CN(C)CCN(C)C(=O)N1CCN(c2cc(S(=O)(=O)NC3(C)CC3)cn3c(-c4nnc(C(F)F)s4)nc(Cl)c23)CC1] | 4-(1-chloro-3-(5-(difluoromethyl)-1,3,4-thiadiazol- 2-yl)-6-(N-(1- methylcyclopropyl)sulfamoyl)imidazo[1,5- a]pyridin-8-yl)-N-(2-(dimethylamino)ethyl)-N- methylpiperazine-1-carboxamide |
| 279a | 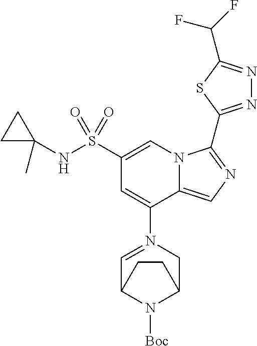 | tert-butyl -3-(3-(5-(difluoromethyl)-1,3,4- thiadiazol-2-yl)-6-(N-(1- methylcyclopropyl)sulfamoyl)imidazo[1,5- a]pyridin-8-yl)-8-azabicyclo[3.2.1]oct-2-ene-8- carboxylate |
| 279b | 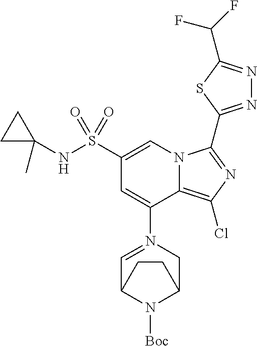 | tert-butyl-3-(1-chloro-3-(5-(difluoromethyl)-1,3,4- thiadiazol-2-yl)-6-(N-(1- methylcyclopropyl)sulfamoyl)imidazo[1,5- a]pyridin-8-yl)-8-azabicyclo[3.2.1]oct-2-ene-8- carboxylate |
| 279 | 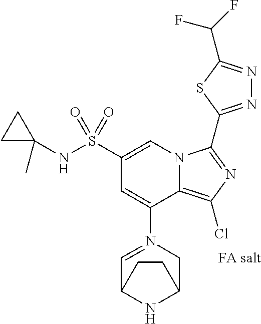 | 8-((1R,5S)-8-azabicyclo[3.2.1]oct-2-en-3-yl)-1- chloro-3-(5-(difluoromethyl)-1,3,4-thiadiazol-2- yl)-N-(1-methylcyclopropyl)imidazo[1,5- a]pyridine-6-sulfonamide formate |
| 280 | 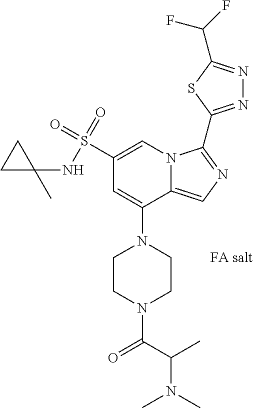 [SMILES: CC(C(=O)N1CCN(c2cc(S(=O)(=O)NC3(C)CC3)cn3c(-c4nnc(C(F)F)s4)ncc23)CC1)N(C)C] | 3-(5-(difluoromethyl)-1,3,4-thiadiazol-2-yl)-8-(4- (dimethyla]anyl)piperazin-1-yl)-N-(1- methylcyclopropyl)imidazo[1,5-a]pyridine-6- sulfonamide formate |
| 281 | 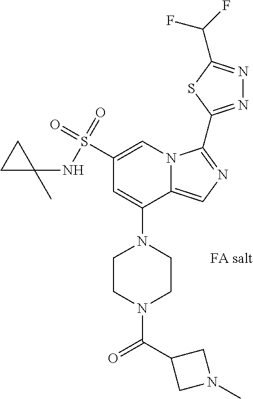 [SMILES: CN1CC(C(=O)N2CCN(c3cc(S(=O)(=O)NC4(C)CC4)cn4c(-c5nnc(C(F)F)s5)ncc34)CC2)C1] | 3-(5-(difluoromethyl)-1,3,4-thiadiazol-2-yl)-8-(4- (1-methylazetidine-3-carbonyl)piperazin-1-yl)-N- (1-methylcyclopropyl)imidazo[1,5-a]pyridine-6- sulfonamide formate |
| 282a | 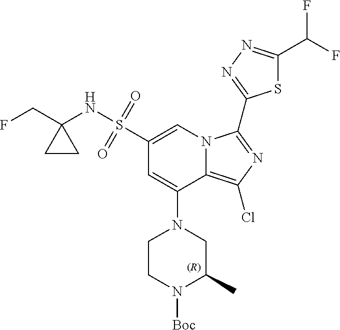 | tert-butyl (R)-4-(1-chloro-3-(5-(difluoromethyl)- 1,3,4-thiadiazol-2-yl)-6-(N-(1- (fluoromethyl)cyclopropyl)sulfamoyl)imidazo[1,5- a]pyridin-8-yl)-2-methylpiperazine-1-carboxylate formate |
| 282 | 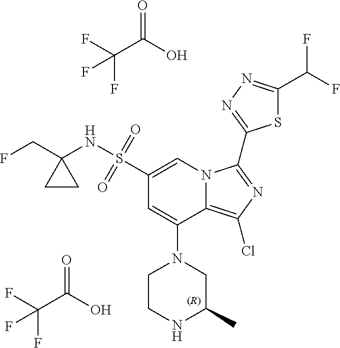 | (R)-1-chloro-3-(5-(difluoromethyl)-1,3,4- thiadiazol-2-yl)-N-(1-(fluoromethyl)cyclopropyl)- 8-(3-methylpiperazin-1-yl)imidazo[1,5-a]pyridine- 6-sulfonamide bis(2,2,2-trifluoroacetate) |
| 283a | 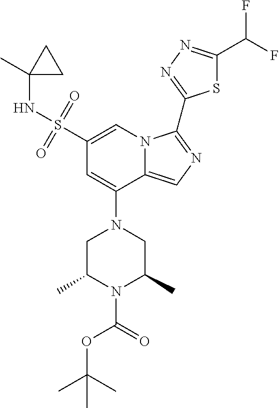 [SMILES: C[C@@H]1CN(c2cc(S(=O)(=O)NC3(C)CC3)cn3c(-c4nnc(C(F)F)s4)ncc23)C[C@@H](C)N1C(=O)OC(C)(C)C] | tert-butyl (2R,6R)-4-(3-(5-(difluoromethyl)-1,3,4- thiadiazol-2-yl)-6-(N-(1- methylcyclopropyl)sulfamoyl)imidazo[1,5- a]pyridin-8-yl)-2,6-dimethylpiperazine-1- carboxylate |
| 283 | 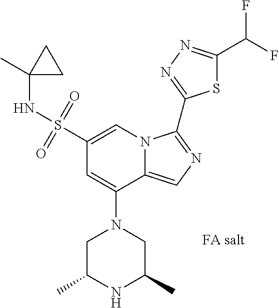 [SMILES: C[C@@H]1CN(c2cc(S(=O)(=O)NC3(C)CC3)cn3c(-c4nnc(C(F)F)s4)ncc23)C[C@@H](C)N1] | 3-(5-(difluoromethyl)-1,3,4-thiadiazol-2-yl)-8- ((3R,5R)-3,5-dimethylpiperazin-1-yl)-N-(1- methylcyclopropyl)imidazo[1,5-a]pyridine-6- sulfonamide formate |
| 284a | 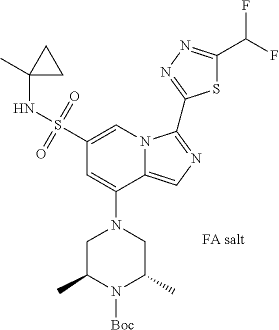 [SMILES: C[C@H]1CN(c2cc(S(=O)(=O)NC3(C)CC3)cn3c(-c4nnc(C(F)F)s4)ncc23)C[C@H](C)N1C(=O)OC(C)(C)C] | tert-butyl (2S,6S)-4-(3-(5-(difluoromethyl)-1,3,4- thiadiazol-2-yl)-6-(N-(1- methylcyclopropyl)sulfamoyl)imidazo[1,5- a]pyridin-8-yl)-2,6-dimethylpiperazine-1- carboxylate |
| 284 | 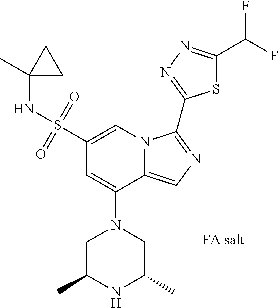 [SMILES: C[C@H]1CN(c2cc(S(=O)(=O)NC3(C)CC3)cn3c(-c4nnc(C(F)F)s4)ncc23)C[C@H](C)N1] | 3-(5-(difluoromethyl)-1,3,4-thiadiazol-2-yl)-8- ((3S,5S)-3,5-dimethylpiperazin-1-yl)-N-(1- methylcyclopropyl)imidazo[1,5-a]pyridine-6- sulfonamide formate |
| 285 | 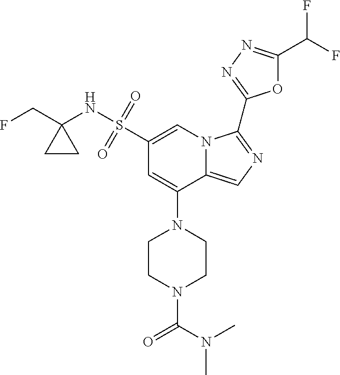 [SMILES: CN(C)C(=O)N1CCN(c2cc(S(=O)(=O)NC3(CF)CC3)cn3c(-c4nnc(C(F)F)o4)ncc23)CC1] | 4-(3-(5-(difluoromethyl)-1,3,4-oxadiazol-2-yl)-6- (N-(1- (fluoromethyl)cyclopropyl)sulfamoyl)imidazo[1,5- a]pyridin-8-yl)-N,N-dimethylpiperazine-1- carboxamide |
| 286a | 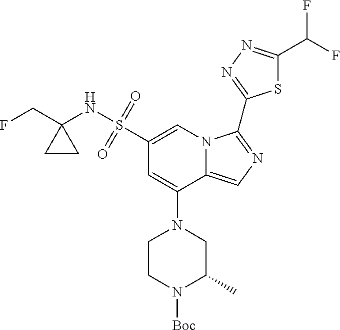 [SMILES: C[C@H]1CN(c2cc(S(=O)(=O)NC3(CF)CC3)cn3c(-c4nnc(C(F)F)s4)ncc23)CCN1C(=O)OC(C)(C)C] | tert-butyl (S)-4-(3-(5-(difluoromethyl)-1,3,4- thiadiazol-2-yl)-6-(N-(1- (fluoromethyl)cyclopropyl)sulfamoyl)imidazo[1,5- a]pyridin-8-yl)-2-methylpiperazine-1-carboxylate |
| 286 | 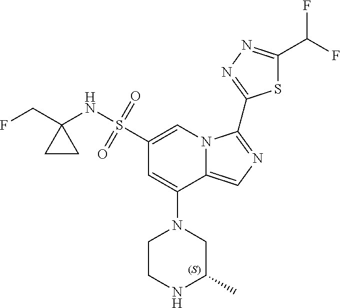 | (S)-3-(5-(difluoromethyl)-1,3,4-thiadiazol-2-yl)-N- (1-(fluoromethyl)cyclopropyl)-8-(3- methylpiperazin-1-yl)imidazo[1,5-a]pyridine-6- sulfonamide |
| 287a | 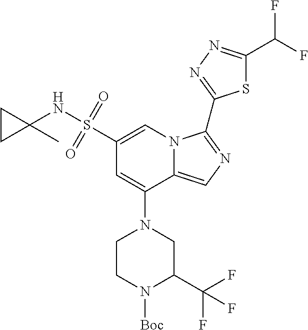 [SMILES: CC1(NS(=O)(=O)c2cc(N3CCN(C(=O)OC(C)(C)C)C(C(F)(F)F)C3)c3cnc(-c4nnc(C(F)F)s4)n3c2)CC1] | tert-butyl 4-(3-(5-(difluoromethyl)-1,3,4-thiadiazol- 2-yl)-6-(N-(1- methylcyclopropyl)sulfamoyl)imidazo[1,5- a]pyridin-8-yl)-2-(trifluoromethyl)piperazine-1- carboxylate |
| 287 | 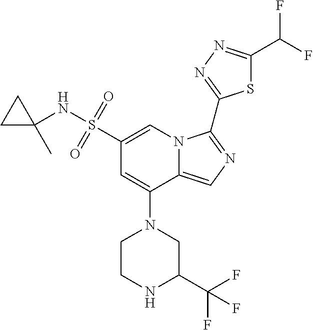 [SMILES: CC1(NS(=O)(=O)c2cc(N3CCNC(C(F)(F)F)C3)c3cnc(-c4nnc(C(F)F)s4)n3c2)CC1] | 3-(5-(difluoromethyl)-1,3,4-thiadiazol-2-yl)-N-(1- methylcyclopropyl)-8-(3- (trifluoromethyl)piperazin-1-yl)imidazo[1,5- a]pyridine-6-sulfonamide |
| 288 | 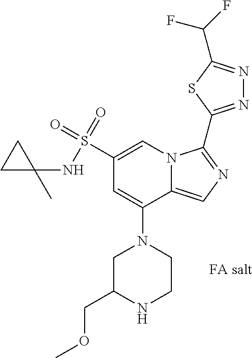 [SMILES: COCC1CN(c2cc(S(=O)(=O)NC3(C)CC3)cn3c(-c4nnc(C(F)F)s4)ncc23)CCN1] | 3-(5-(difluoromethyl)-1,3,4-thiadiazol-2-yl)-8-(3- (methoxymethyl)piperazin-1-yl)-N-(1- methylcyclopropyl)imidazo[1,5-a]pyridine-6- sulfonamide formate |
| 289 | 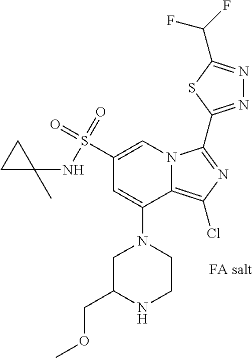 [SMILES: COCC1CN(c2cc(S(=O)(=O)NC3(C)CC3)cn3c(-c4nnc(C(F)F)s4)nc(Cl)c23)CCN1] | 1-chloro-3-(5-(difluoromethyl)-1,3,4-thiadiazol-2- yl)-8-(3-(methoxymethyl)piperazin-1-yl)-N-(1- methylcyclopropyl)imidazo[1,5-a]pyridine-6- sulfonamide formate |
| 290 | 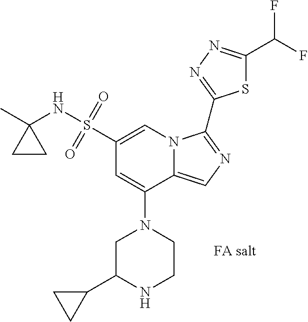 | 8-(3-cyclopropylpiperazin-1-yl)-3-(5- (difluoromethyl)-1,3,4-thiadiazol-2-yl)-N-(1- methylcyclopropyl)imidazo[1,5-a]pyridine-6- sulfonamide formate |
| 291 | 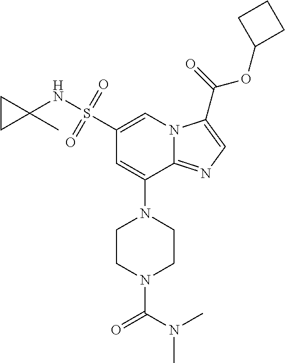 [SMILES: CN(C)C(=O)N1CCN(c2cc(S(=O)(=O)NC3(C)CC3)cn3c(C(=O)OC4CCC4)cnc23)CC1] | cyclobutyl 8-(4-(dimethylcarbamoyl)piperazin-1- yl)-6-(N-(1- methylcyclopropyl)sulfamoyl)imidazo[1,2- a]pyridine-3-carboxylate |
| 292 | 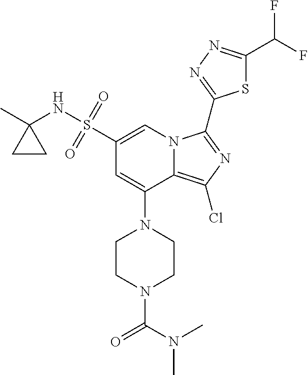 [SMILES: CN(C)C(=O)N1CCN(c2cc(S(=O)(=O)NC3(C)CC3)cn3c(-c4nnc(C(F)F)s4)nc(Cl)c23)CC1] | 4-(1-chloro-3-(5-(difluoromethyl)-1,3,4-thiadiazol- 2-yl)-6-(N-(1- methylcyclopropyl)sulfamoyl)imidazo[1,5- a]pyridin-8-yl)-N,N-dimethylpiperazine-1- carboxamide |
| 293 | 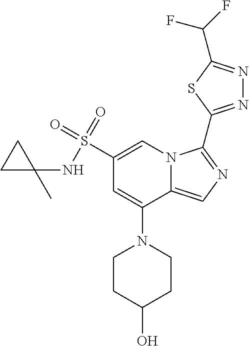 [SMILES: CC1(NS(=O)(=O)c2cc(N3CCC(O)CC3)c3cnc(-c4nnc(C(F)F)s4)n3c2)CC1] | 3-(5-(difluoromethyl)-1,3,4-thiadiazol-2-yl)-8-(4- hydroxypiperidin-1-yl)-N-(1- methylcyclopropyl)imidazo[1,5-a]pyridine-6- sulfonamide |
| 39 FA salt | 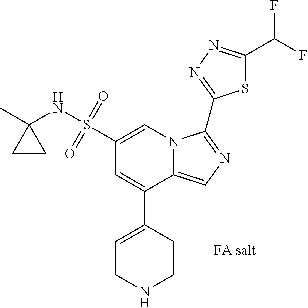 | 3-(5-(difluoromethyl)-1,3,4-thiadiazol-2-yl)-N-(1- methylcyclopropyl)-8-(1,2,3,6-tetrahydropyridin- 4-yl)imidazo[1,5-a]pyridine-6-sulfonamide formate |
| 295 | 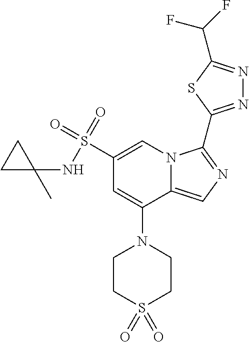 [SMILES: CC1(NS(=O)(=O)c2cc(N3CCS(=O)(=O)CC3)c3cnc(-c4nnc(C(F)F)s4)n3c2)CC1] | 3-(5-(difluoromethyl)-1,3,4-thiadiazol-2-yl)-8-(1,1- dioxidothiomorpholino)-N-(1- methylcyclopropyl)imidazo[1,5-a]pyridine-6- sulfonamide |
| 296 | 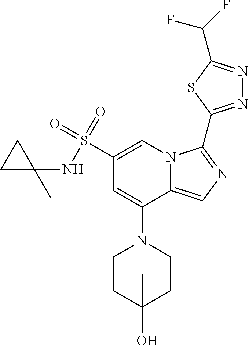 | 3-(5-(difluoromethyl)-1,3,4-thiadiazol-2-yl)-8-(4- hydroxy-4-methylpiperidin-1-yl)-N-(1- methylcyclopropyl)imidazo[1,5-a]pyridine-6- sulfonamide |
| 297 | 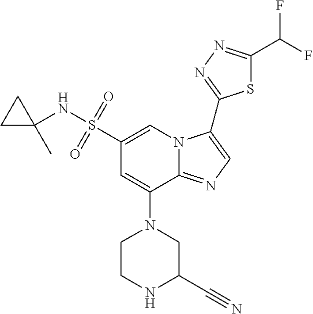 [SMILES: CC1(NS(=O)(=O)c2cc(N3CCNC(C#N)C3)c3ncc(-c4nnc(C(F)F)s4)n3c2)CC1] | 8-(3-cyanopiperazin-1-yl)-3-(5-(difluoromethyl)- 1,3,4-thiadiazol-2-yl)-N-(1- methylcyclopropyl)imidazo[1,5-a]pyridine-6- sulfonamide |
| 298 | 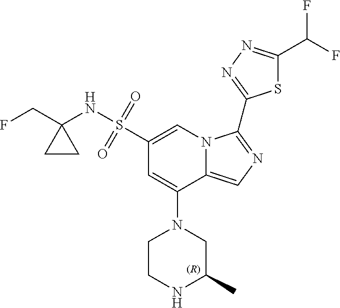 | (R)-3-(5-(difluoromethyl)-1,3,4-thiadiazol-2-yl)-N- (1-(fluoromethyl)cyclopropyl)-8-(3- methylpiperazin-1-yl)imidazo[1,5-a]pyridine-6- sulfonamide |
| 299 | 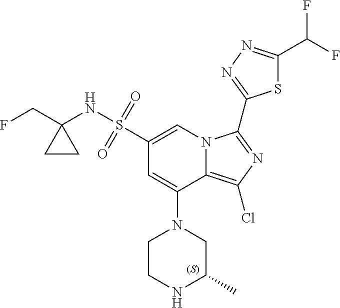 | (S)-1-chloro-3-(5-(difluoromethyl)-1,3,4- thiadiazol-2-yl)-N-(1-(fluoromethyl)cyclopropyl)- 8-(3-methylpiperazin-1-yl)imidazo[1,5-a]pyridine- 6-sulfonamide |

### Biological Evaluation of the Exemplary Compounds

Exemplary compounds of formula (I) were tested in selected biological and/or physicochemical assays one or more times. When tested more than once, data are reported as either average values or as median values, wherein the average value, also referred to as the arithmetic mean value, represents the sum of the values obtained divided by the number of times tested, and the median value represents the middle number of the group of values when ranked in ascending or descending order. If the number of values in the data set is odd, the median value is the middle value. If the number of values in the data set is even, the median is the arithmetic mean of the two middle values. The in vitro pharmacological, pharmacokinetic and physicochemical properties of the compounds can be determined according to the following assays and methods.

### PARG Protein Expression and Purification

A codon optimized gene encoding human PARG (448-976 [H446G, L447S, L473S, N479S, S802A, R811K, M841I, S858P, I916T, T924D, D927K, C963S, A967T]) was synthesized by Genscript, and cloned into pET15b (NcoI/BamHI) with an N-terminal, Thrombin protease cleavable 6His-TwinStrep tag. Expression of the protein in E. coli BL21 (DE3) was induced by addition of 0.2 mM IPTG to a shake flask culture grown to OD600=0.8 at 37° C. Growth was allowed to continue at 30° C. for a further 20 hours before harvesting by centrifugation and storage of the cell pellet at −80° C.

Protein was purified by IMAC and SEC: frozen cell pellets (typically 40 g wet weight) were resuspended by homogenization in 5 volumes buffer A (25 mM Tris/HCl pH 8.0, 200 mM NaCl, 2 mM DTT), supplemented with 1 mg of DNase I from bovine pancreas (Sigma-Aldrich) and protease inhibitors (Roche Complete™ EDTA-free protease inhibitor tablet), and lysed by passage through a Constant Systems BasicZ homogenizer. The lysate was clarified by centrifugation for 60 minutes at 25,000 g, 4° C., and the lysate supernatant was loaded onto 5 ml StrepTrap HP (Cytiva) pre-equilibrated with buffer A. The column was washed with buffer A (˜10 CV), then buffer B containing 1M KCl (˜5 CV), and then the protein was eluted with buffer A containing 2.5 mM d-Desthiobiotin. Pooled fractions containing 6HisTwinStrep-TEV-hPARG were incubated with TEV protease overnight at 4° C. hPARG was separated from uncleaved material and Thrombin protease through gel filtration with Superdex75 sizing column (GE Healthcare) pre-equilibrated with SEC buffer (15 mM Tris/HCl pH 8.5, 100 mM NaCl, 2 mM DTT). Pooled fractions containing pure hPARG were concentrated using a 10 k MWCO spin concentrator (VivaSpin) to 10 mg/mL, and then either used immediately for crystallisation or snap-frozen in liquid nitrogen for storage at −80° C.

### PARG Enzymatic IC₅₀ Assay

PARG enzyme as incubated with compound or vehicle (DMSO) for 15 minutes or 2 hours in a 384 well plate. After adding the PARG substrate ADP-ribose-pNP, the plate was read for absorbance intensity at 405 nm. The vehicle (DMSO) with high absorbance intensity represents no inhibition of enzymatic reaction while the low control (no enzyme) with low absorbance intensity represents full inhibition of enzymatic reaction.

### Materials:

hPARG: Peak Protein, 30 nM
Substrate: ADP-pNP, 800 μM, Jena Bioscience catalog #NU-955
Reaction time: 60 minutes
Assay buffer: 50 mM Tris-HCl pH 8.0, 100 mM NaCl, 2 mM DTT
Temperature: 30° C.
Total volume: 30 μL

### Controls:

0% inhibition control: DMSO
100% inhibition control: No enzyme

The protocol that was used for enzyme reaction and detection is as follows:
1. Transfer 100 nL of the final concentration of test compounds or vehicle (DMSO) to the appropriate wells of a microtiter plate.
2. Centrifuge the plate at 1000 rpm for 1 minute.
3. Transfer 14.6 μL of 2× final concentration of enzyme in assay buffer or assay buffer alone to the appropriate wells.
4. Centrifuge the plate at 1000 rpm for 1 minute.
5. Incubate the plate at room temperature for 15 minutes or 2 hours.
6. Transfer 15.4 μL of 2× substrate in assay buffer to all the test wells.
7. Centrifuge the plate at 1000 rpm for 1 minute.
8. Read the plate on a plate reader (e.g., Spark Tecan).
The Absorbance IC₅₀ value of compounds of Formula (I) in Examples 1 to 299 are provided in Table 2 below.

### Cellular PAR Chain Assay

The ability of compounds to inhibit PARG in response to DNA damage, was assessed with U2OS cells pretreated with the compounds for 1 hour, following a 1-hour treatment with or without the DNA alkylating agent temozolomide (TMZ). The cells were harvested and fixed in 70% ethanol, rehydrated with glucose and EDTA in PBS and subsequently blocked for 1 hour with PBS 1% BSA and 0.01% Tween-20 (PBT). The cells were incubated for 2 hours at room temperature with a mouse monoclonal antibody against poly (ADP) ribose (PAR) polymer. The cells were washed and incubated with an anti-mouse Alexa-488 conjugated secondary antibody for 1 hour at room temperature. Propidium iodide staining was used to determine DNA content in the cells (staining at 4° C. overnight). The fluorescence intensity of the cells was assessed by flow cytometry (Cytoflex from Beckmann) and the percentage of PAR chain positive cells (gated in relation to TMZ+DMSO treated control) was determined. PAR chain positive cells % were fit against the concentration of the compound using a 4 parameter log-logistic function, generating PAR chain EC₅₀ values:

$$f ( x ) = c + \frac{d - c}{1 + {exp}^{b ( log ( x ) - log ( e ) )}}$$

The PAR chain EC₅₀ value for compounds of Formula (I) in Examples 1 to 25 are provided in Table 2 below.

### Cellular Viability Assay

NCIH-460 as a PARG-inhibition sensitive cell line and U2OS as PARG-inhibition insensitive cell line were plated at 1000 cells/well and 2000 cells/well, respectively, in 96-well white plates with clear flat bottom. After 24 hours, the compounds were added with the Tecan digital dispenser (D300e), in duplicates. The outer wells of the plate were excluded. After 96 hours of incubation, 150 μl of the growth medium were removed and 50 μl of Cell Titer-Glo (Promega) were added per well. Following an incubation of 10 minutes, luminescence was read using a plate reader (Tecan). Averaged values of the samples were normalized to DMSO treated control samples. Curves were fit as % of the control vs. log of the compound concentration using a 4 parameter log-logistic function:

$$f ( x ) = c + \frac{d - c}{1 + {exp}^{b ( log ( x ) - log ( e ) )}}$$

The PARGi (NCIH-460 and U2OS) cellular viability EC₅₀ values for compounds of Formula (I) in Examples 1 to 299 are provided in Table 2 below.

Table 2: Inhibition of PARG and cellular activity of compounds according to the present invention.

The IC₅₀ (inhibitory concentration at 50% of maximal effect) values are indicated in μM, empty space means that the corresponding compounds have not been tested in the respective assay.
\textcircled{1} Example number
\textcircled{3} IC₅₀ in μM determined in PARG enzymatic assay (PARG protein and 15 mn incubation) described under PARG enzymatic IC₅₀ assay
\textcircled{4} IC₅₀ in μM determined in PARG enzymatic assay (PARG protein and 2 hours incubation) described under PARG enzymatic IC₅₀ assay
\textcircled{5}. EC₅₀ in μM determined in cellular assay as described under Cellular PAR chain assay (conditions with treatment of TMZ).
\textcircled{6} EC₅₀ in μM determined in cellular assay as described under Cellular PAR chain assay (conditions without treatment of TMZ).
\textcircled{7} EC₅₀ in μM determined in NCIH-460 cells as described under Cellular viability assay.
\textcircled{8} EC₅₀ in μM determined in U2OS cells as described under Cellular viability assay.

TABLE 2

| \textcircled{1} | \textcircled{3} | \textcircled{4} | \textcircled{5} | \textcircled{6} | \textcircled{7} | \textcircled{8} |
|---|---|---|---|---|---|---|
| 1 | 0.793 | | 100 | 100 | 100 | 100 |
| 2 | 0.016 | | 0.129 | 100 | 2.85 | 100 |
| 3 | 0.058 | | 0.986 | 100 | 3.679 | 100 |
| 4 | 0.415 | | | | | |
| 5 | 0.024 | | 0.195 | 100 | 4.522 | 100 |
| 6 | 0.192 | | 5.303 | 100 | 33.524 | 26.256 |
| 7 | 0.016 | | 0.17 | 1.58 | 1.554 | 100 |
| 8 | 0.03 | | 0.375 | 100 | 13.813 | 100 |
| 9 | 0.3 | | 24.853 | 100 | 100 | 100 |
| 10 | 0.806 | | | | | |
| 11 | 1.141 | | 100 | 100 | 100 | 100 |
| 13 | | 0.074 | 0.053 | 86.99 | 0.605 | >30 |
| 14 | 0.019 | 0.02 | <0.02 | >30 | 0.239 | >30 |
| 15 | | 0.03 | 0.546 | >30 | 3.723 | >30 |
| 16 | | 0.038 | 11.681 | >30 | 4.604 | >30 |
| 17 | | 0.021 | 0.443 | >30 | 3.557 | >30 |
| 18 | | 0.024 | 0.07 | 3.67 | 0.75 | 24.55 |
| 19 | | 0.252 | 0.353 | >10 | 4.748 | >20 |
| 20 | | 0.017 | 0.082 | 17.919 | 0.829 | 24.812 |
| 21 | | 0.027 | 0.048 | 0.724 | 0.62 | 7.16 |
| 22 | | 0.022 | 0.02 | 1.84 | 0.37 | 12.47 |
| 23 | | 0.016 | 0.017 | 0.95 | 0.26 | 10.09 |
| 24 | | 0.01 | 0.029 | 1.21 | 0.36 | 14.13 |
| 25 | | 0.013 | <0.004 | 0.07 | <0.1 | 22.71 |
| 26 | | 0.03 | | | 0.77 | >20 |
| 27 | | 0.011 | <0.003 | 2.263 | 0.2 | 17.344 |
| 28 | | 0.015 | 0.003 | 0.485 | 0.057 | >20 |
| 29 | | 0.011 | <0.003 | 1.269 | 0.015 | 18.935 |
| 31 | | 0.022 | 0.0165 | 0.601 | 0.4047 | >18.937 |
| 32 | | 0.01 | <0.0045 | 1.28 | 0.077 | >20 |
| 33 | | 0.027 | | | 1.22 | >20 |
| 34 | | 0.029 | 0.352 | 0.553 | 0.859 | 16.832 |
| 35 | | 0.011 | 0.006 | 0.135 | 0.085 | 7.215 |
| 36 | | 0.011 | <0.003 | 7.478 | <0.0245 | 16.128 |
| 37 | | 0.01 | <0.003 | 0.258 | <0.034 | >7.75 |
| 38 | | 0.012 | <0.004 | 0.23 | <0.092 | 15.869 |
| 39 | | | | | | |
| 40 | | 0.01 | <0.022 | 1.509 | <0.057 | >16.597 |
| 41 | | 0.013 | <0.02 | 1.61 | <0.12 | >30 |
| 42 | | 0.011 | 0.022 | >10 | 0.1045 | >20 |
| 43 | | 0.011 | 0.408 | >10 | 3.386 | >20 |
| 44 | | 0.009 | <0.004 | 1.29 | <0.115 | >28.545 |
| 45 | | 0.014 | 2.443 | >10 | 6.047 | >20 |
| 46 | | 0.066 | | | | |
| 47 | | 0.01 | 0.006 | >10 | 0.087 | 14.209 |
| 48 | | 0.017 | 0.081 | >10 | 1.033 | >20 |
| 49 | | 0.01 | 0.049 | >10 | 1.967 | >20 |
| 50 | | 0.011 | 0.008 | >10 | 0.494 | 6.681 |
| 51 | | 0.01 | 0.009 | 1.077 | 0.672 | >20 |
| 52 | | 0.072 | 5.61 | >10 | 19.12 | >20 |
| 53 | | 0.117 | | | | |
| 54 | | 0.064 | | | | |
| 55 | | 0.039 | 0.586 | >10 | 3.908 | >20 |
| 56 | | 0.016 | 0.3 | >10 | 1.59 | >20 |
| 57 | | 0.033 | 0.385 | >10 | 0.956 | >20 |
| 58 | | 0.027 | 0.492 | >10 | 1.507 | >20 |
| 59 | | 0.879 | | | | |
| 60 | | 0.011 | 0.359 | >10 | 6.498 | 10.334 |
| 61 | | 0.023 | 0.265 | >10 | 0.735 | 18.142 |
| 62 | | 0.03 | 0.357 | >10 | 5.057 | 9.083 |
| 63 | | 0.254 | | | | |
| 64 | | 0.105 | | | | |
| 65 | | 0.084 | 2.43 | >30 | 3.99 | >30 |
| 66 | | 0.062 | | | 10.73 | >20 |
| 67 | | 0.038 | | | 2.665 | 3.559 |
| 68 | | 0.08 | 0.292 | >10 | 0.454 | 5.874 |
| 69 | | 0.129 | | | | |
| 70a | | 0.022 | | | 1.21 | >20 |
| 70 | | 0.011 | 0.009 | 0.302 | 0.452 | >13.34 |
| 71 | | 0.01 | 0.07 | >10 | <0.0445 | 20.272 |
| 72 | | 0.027 | 0.149 | >10 | 3.103 | >20 |
| 73 | | 0.011 | 0.1425 | >10 | 0.9125 | >20 |
| 74 | | 0.025 | 0.355 | >10 | 2.069 | >20 |
| 78 | | 0.35 | | | | |
| 79 | | 0.008 | 0.426 | >10 | 2.827 | >20 |
| 81 | | 0.022 | | | 3.969 | >20 |
| 82 | | 0.011 | 2.637 | >10 | >20 | >20 |
| 83 | | 0.511 | | | | |
| 84 | | 3.379 | | | | |
| 85 | | 0.422 | | | | |
| 86 | | 0.058 | | | >30 | >30 |
| 87 | | 0.17 | 2.44 | 12.29 | >30 | >30 |
| 88 | | 0.018 | 1.889 | >10 | >20 | >20 |
| 89 | | 7.3 | | | | |
| 90 | | 0.044 | | | >20 | >20 |
| 91 | | 0.041 | | | 10.39 | >20 |
| 92 | | 0.011 | | | 21.554 | >20 |
| 93 | | 0.15 | | | | |
| 94 | | 0.021 | 2.495 | >10 | >20 | >20 |
| 95 | | 0.029 | | | 1.799 | >20 |
| 96 | | 0.169 | | | | |
| 97 | | 0.064 | | | | |
| 98 | | 0.063 | 3.416 | >10 | >20 | >20 |
| 99 | | 0.27 | | | | |
| 100 | | 0.067 | | | | 0.067 |
| 101 | | 0.07 | | | | 0.07 |
| 102 | | 0.057 | >10 | >10 | >20 | 0.057 |
| 103 | | 0.375 | | | | 0.375 |
| 105 | | 0.719 | | | | |
| 106 | | 0.610 | | | | |
| 107 | | 0.119 | | | | |
| 108 | | 0.323 | | | | |
| 109 | | 0.145 | | | | |
| 110 | | 0.153 | | | | |
| 111 | | 1.92 | | | | |
| 113 | | 0.013 | 0.5 | >30 | 2.422 | >30 |
| 114 | | 0.01 | 0.03 | 3.67 | 0.496 | >30 |
| 117 | | 0.017 | 0.018 | 11.91 | 0.406 | 25.278 |
| 119 | | 0.014 | 0.019 | 18.61 | 0.3335 | 23.354 |
| 120 | | 0.012 | 0.004 | 0.757 | 0.084 | 16.404 |
| 121 | | 0.069 | 0.533 | >10 | 11.623 | 14.613 |
| 122 | | 0.014 | | | 4.096 | >20 |
| 123 | | 0.015 | 0.197 | >10 | 1.38 | 9.084 |
| 124 | | 0.02 | 0.06 | 0.572 | 1.712 | 5.524 |
| 125 | | 0.583 | | | | |
| 126 | | 0.065 | 0.576 | >10 | 11.031 | >20 |
| 127 | | 0.058 | | | | |
| 128 | | 0.032 | 0.043 | 3.478 | 1.163 | 6.687 |
| 129 | | 0.012 | <0.004 | 1.08 | <0.145 | 19.715 |
| 130 | | 0.017 | <0.003 | 2.403 | 0.886 | 21.231 |
| 131 | | 0.094 | | | | |
| 132 | | 0.096 | | | | |
| 133 | | 0.021 | | | 1.05 | 16.97 |
| 134 | | 0.01 | | | 0.47 | 16.01 |
| 135 | | 0.011 | 0.029 | 4.069 | 0.654 | >20 |
| 136 | | 0.011 | | | 1.62 | >20 |
| 137 | | 0.021 | 0.03 | 0.469 | 0.317 | >20 |
| 138 | | 0.015 | 0.109 | >10 | 1.938 | >20 |
| 140 | | 0.029 | 0.18 | >10 | 1.124 | >20 |
| 141 | | 0.023 | | | 1.71 | >20 |
| 143 | | 0.026 | 1.12 | >21.655 | 3.75 | >30 |
| 146 | | 0.013 | <0.003 | 0.263 | 0.079 | >20 |
| 148 | | | | | | |
| 151 | | 0.014 | 1.087 | 14.21 | 5.3187 | >23.3 |
| 152 | | 0.018 | 0.247 | >10 | 2.056 | >20 |
| 153 | | 0.011 | 0.197 | >20 | 1.468 | >20 |
| 154 | | 0.04 | | | | |
| 155 | | 0.021 | 0.02 | 1.17 | 1.35 | 16.27 |
| 156 | | 0.138 | 0.446 | <10 | 0.625 | >13.7 |
| 157 | | 0.252 | 0.353 | >10 | 4.748 | >20 |
| 158 | | 0.028 | 0.097 | 5.83 | 1.005 | 14.32 |
| 159 | | 0.254 | 5.36 | >30 | 4.74 | 6.68 |
| 160 | | 0.093 | 5.213 | >30 | 20.55 | >30 |
| 161 | | 0.068 | 0.89 | 29.58 | 4.69 | >30 |
| 162 | | 0.01 | 0.02 | 8.924 | 0.433 | >20 |
| 163 | | 0.039 | 0.151 | 3.538 | 0.869 | >20 |
| 164 | | 0.009 | 0.037 | 0.98 | 0.46 | 12.28 |
| 165 | | 0.016 | 0.078 | 0.916 | 0.496 | 13.292 |
| 166 | | 0.07 | | | 1.35 | 19.59 |
| 168 | | 0.011 | 0.039 | >10 | 0.825 | >20 |
| 169 | | 4.911 | | | | |
| 170 | | 6.1 | | | | |
| 171 | | 0.03 | | | 5.359 | >20 |
| 172 | | 0.382 | | | | |
| 173 | | 0.01 | 0.008 | 4.955 | 0.128 | 3.663 |
| 174 | | 8.43 | | | | |
| 175 | | 0.01 | 0.391 | >10 | 1.903 | >20 |
| 176 | | 0.039 | 0.316 | 1.434 | 7.899 | 20.54 |
| 177b | | 0.012 | 0.007 | 0.229 | 0.139 | 6.289 |
| 177 | | 0.011 | 0.005 | 2.636 | 0.265 | >13.7 |
| 178a | | 0.035 | 0.103 | 2.225 | 0.959 | 21.723 |
| 178 | | 0.011 | <0.004 | 0.618 | 0.07 | >13.3 |
| 179 | | 0.022 | 0.031 | 3.59 | 0.171 | >17.8 |
| 180 | | 0.012 | 0.008 | 0.147 | 0.294 | 7.212 |
| 181 | | 0.015 | 0.011 | 0.341 | 0.345 | 7.244 |
| 182 | | 0.011 | 0.005 | 0.218 | 0.164 | 8.181 |
| 183 | | 0.01 | 0.078 | 1.253 | 0.628 | 3.289 |
| 184 | | 0.017 | <0.004 | 0.197 | 0.068 | 7.128 |
| 185 | | 0.011 | 0.035 | 1.057 | 1.969 | 7.483 |
| 187 | | 0.011 | 0.013 | 7.317 | 0.318 | 24.397 |
| 188 | | 0.011 | <0.003 | >10 | 0.064 | >20 |
| 189 | | 0.011 | 0.006 | 0.1335 | 0.219 | 5.552 |
| 190 | | 0.011 | 0.087 | 0.81 | 4.898 | >20 |
| 191 | | 0.016 | 0.009 | 0.191 | 0.169 | 5.157 |
| 192 | | 0.015 | 0.006 | 0.098 | 0.146 | 3.045 |
| 194 | | 0.016 | 0.015 | 0.969 | 0.134 | 12.481 |
| 195 | | 0.024 | 0.047 | >10 | 0.589 | >20 |
| 196 | | 0.014 | 0.035 | 0.999 | 0.476 | 15.83 |
| 197a | | 0.061 | | | | |
| 197 | | 0.011 | 0.009 | >5.459 | 0.2345 | >13.285 |
| 198 | | 0.011 | 0.014 | 2.225 | 0.058 | >20 |
| 199 | | 0.009 | 0.005 | 1.394 | 0.071 | >17.71 |
| 200 | | 0.017 | <0.003 | 0.205 | <0.022 | 21.38 |
| 201 | | 0.027 | | | 3.274 | >20 |
| 202 | | 0.028 | | | 1.491 | 8.066 |
| 203 | | 0.028 | 0.009 | >10 | 0.735 | 1.774 |
| 204 | | 0.01 | 0.101 | >10 | 0.436 | >20 |
| 205 | | 0.011 | 0.012 | >10 | 0.4 | 14.702 |
| 206 | | 0.011 | 0.014 | >10 | 0.292 | >20 |
| 207 | | 0.025 | 0.071 | >10 | 0.588 | 3.261 |
| 208 | | 0.011 | | | 0.478 | >20 |
| 209 | | 0.011 | | | 0.606 | 2.074 |
| 210 | | 0.014 | 0.152 | >10 | 0.279 | 13.202 |
| 211 | | 0.077 | | | | |
| 212 | | 0.25 | | | | |
| 213 | | 0.018 | 0.03 | 1.007 | 0.342 | 11.527 |
| 214 | | 0.029 | 0.383 | 1.852 | 5.485 | 6.317 |
| 215 | | 0.011 | | | 2.267 | >20 |
| 216 | | 0.026 | 0.029 | 0.514 | 1.305 | 5.629 |
| 217 | | 0.033 | | | | |
| 218 | | 0.042 | 0.247 | 1.387 | 4.169 | 9.066 |
| 219 | | 0.029 | 0.075 | 0.378 | 1.5 | 6.354 |
| 220 | | 0.029 | 0.14 | 0.38 | 1.126 | 2.202 |
| 221 | | 0.021 | 0.02 | 1.37 | 0.478 | >20 |
| 222 | | 0.027 | 0.049 | 0.286 | 0.926 | 1.379 |
| 223 | | 0.011 | 0.017 | >10 | 0.436 | >20 |
| 224 | | 0.03 | 0.028 | 0.269 | 1.567 | 6.268 |
| 225 | | 0.046 | 0.227 | 1.247 | 2.549 | 6.945 |
| 226 | | 0.034 | 0.102 | 0.738 | 2.313 | 5.348 |
| 227 | | 0.043 | 0.086 | 0.528 | 1.086 | 3.408 |
| 228 | | 0.025 | 0.038 | 0.393 | 0.553 | 5.412 |
| 229 | | 0.106 | | | | |
| 230 | | 0.093 | | | | |
| 231 | | 0.045 | 0.087 | 0.264 | 1.506 | 5.029 |
| 232 | | 0.01 | 0.07 | >10 | 0.944 | >20 |
| 233 | | 0.032 | | | 0.672 | 20.9 |
| 234 | | 0.01 | 0.045 | 7.997 | 1.023 | >20 |
| 235 | | 0.043 | 0.093 | 0.731 | 1.395 | 9.521 |
| 236 | | 0.021 | 0.041 | 0.537 | 0.957 | 8.688 |
| 237 | | 0.018 | | | 0.546 | >20 |
| 238 | | 0.032 | 0.063 | 0.662 | 0.988 | 6.54 |
| 239 | | 0.054 | | | | |
| 240 | | 0.024 | 0.239 | 0.623 | 3.626 | 5.116 |
| 241 | | 0.048 | 0.092 | 0.326 | 1.737 | 2.626 |
| 242 | | 0.027 | 0.021 | 0.243 | 0.685 | 2.042 |
| 243 | | 0.027 | 0.014 | 0.346 | 0.465 | 7.047 |
| 244 | | 0.309 | | | | |
| 245 | | 0.037 | | | 21.141 | >20 |
| 246 | | 0.031 | | | >20 | >20 |
| 247 | | 0.045 | | | >20 | >20 |
| 248 | | 0.033 | | | 0.873 | >20 |
| 249 | | 0.011 | 0.056 | >10 | 0.485 | >20 |
| 250 | | 0.718 | | | | |
| 251 | | 0.065 | | | | |
| 252 | | 0.011 | 0.017 | 0.414 | 0.299 | 6.721 |
| 253 | | 0.021 | 0.044 | >10 | 0.249 | >20 |
| 255 | | 0.055 | 0.295 | 2.335 | 1.749 | >20 |
| 256 | | 0.023 | 0.017 | 0.468 | 0.378 | 14.371 |
| 257 | | 0.022 | 0.106 | >10 | 0.387 | >20 |
| 258 | | 0.203 | | | | |
| 259 | | 0.011 | 1.848 | >10 | 4.736 | 6.509 |
| 260 | | 0.03 | | | 1.404 | 5.284 |
| 261 | | 0.025 | 0.051 | >10 | 0.403 | >20 |
| 262 | | 0.057 | 0.383 | >10 | 8.905 | >20 |
| 263 | | 0.774 | | | | |
| 264 | | 0.011 | 0.187 | >5.376 | 2.463 | >20 |
| 265 | | 0.114 | | | | |
| 266 | | 0.01 | 0.011 | >10 | 0.251 | >20 |
| 267 | | 0.011 | 0.023 | >10 | 0.809 | >20 |
| 268 | | 0.019 | 0.252 | >10 | 2.499 | 11.81 |
| 269 | | 0.035 | 0.295 | >10 | 1.034 | >20 |
| 270 | | 0.04 | 0.41 | 2.165 | >20 | >20 |
| 271 | | 0.027 | 0.032 | 1.638 | 0.832 | >20 |
| 272 | | 0.044 | 0.029 | 0.36 | 0.831 | >20 |
| 273 | | 0.125 | | | | |
| 274 | | 0.015 | 0.005 | 0.697 | 0.158 | 13.203 |
| 275 | | 0.04 | 0.114 | >10 | 0.961 | 4.365 |
| 276 | | 0.069 | 1.509 | >10 | 4.155 | 3.653 |
| 277 | | 0.011 | 0.013 | 0.409 | 0.14 | 3.94 |
| 278 | | 0.017 | <0.003 | 0.028 | 0.049 | 3.261 |
| 279 | | 0.02 | 0.006 | 0.049 | 0.389 | 6.515 |
| 280 | | 0.02 | 0.009 | 0.314 | 0.223 | 6.151 |
| 281 | | 0.062 | 0.045 | 2.488 | 0.976 | >20 |
| 282 | | 0.025 | 0.005 | 0.137 | 0.095 | 7.222 |
| 283 | | 0.009 | 0.004 | 0.022 | 0.566 | 17.096 |
| 284 | | 0.011 | <0.003 | <0.003 | <0.019 | 11.9 |
| 285 | | 0.022 | 0.072 | >10 | 2.599 | >20 |
| 286 | | 0.012 | 0.004 | 0.068 | 0.076 | 10.592 |
| 287 | | 0.029 | 0.079 | 2.242 | 0.819 | >20 |
| 288 | | 0.017 | 0.004 | 0.089 | 0.071 | 20.115 |
| 289 | | 0.016 | 0.006 | 0.1 | 0.063 | 6.704 |
| 290 | | 0.03 | 0.008 | 0.305 | 0.177 | 6.636 |
| 291 | | 0.04 | 0.096 | >10 | 0.556 | >20 |
| 292 | | 0.024 | <0.003 | 0.192 | <0.019 | 10.394 |
| 293 | | 0.012 | 0.062 | 1.457 | 0.484 | >20 |
| 39 | | 0.011 | 0.022 | 0.403 | 0.234 | 8.947 |
| FA salt | | | | | | |
| 295 | | 0.011 | | | | |
| 296 | | 0.014 | | | | |
| 297 | | 0.011 | 0.019 | 1.206 | 3.207 | >20 |
| 298 | | 0.011 | <0.003 | 0.042 | 0.122 | 9.02 |
| 299 | | 0.017 | 0.005 | 0.073 | 0.116 | 6.387 |

### Further Assays

### Kinetic Solubility Assay

The Kinetic solubility assay employs the shake flask method followed by HPLC-UV analysis. For exemplary compounds, the kinetic solubility was measured according to the following protocol:
1) Samples were weighed and dissolved in 100% DMSO to make a stock solution of 10 mM. About 100 μL of stock solution is needed to cover this assay.
2) Test compounds and controls (10 mM in DMSO, 10 μL/tube) were added into the buffer (490 μL/well) which placed in a Minni-Uniprep filter. The buffer was prepared as the customer's requirement.
3) Vortex the kinetic solubility samples for 2 minutes.
4) Incubate and shake the solubility solutions on an orbital shaker for 24 hr at room temperature
5) Transfer 200 μL each of solubility solution into 96-deep well for analysis when the samples were directly filtered by the syringeless filter device
6) Determine the test compound concentration of the filtrate using HPLC-UV.
7) Injected three UV standard solutions into HPLC from low to high concentration, followed by testing of the K.S. supernatant. Testing samples are injected in duplicate.

### Bidirectional Permeability in Caco2

The bidirectional permeability in Caco-2 cells assay was performed for the exemplary compounds of formula (I) according to the following protocol:
1. Caco-2 cells purchased from ATCC were seeded onto polyethylene membranes (PET) in 96-well BD Insert plates at 1×105 cells/cm2, and refreshed medium every 4˜5 days until to the 21st to 28th day for confluent cell monolayer formation.
2. The integrity of the monolayer is verified by performing Lucifer yellow rejection assay.
3. The quality of the monolayer is verified by measuring the Unidirectional (A→B) permeability of fenoterol/nadolol (low permeability marker), propranolol/metopronolol (high permeability marker) and Bi-directional permeability of Digoxin (a P-glycoprotein substrate marker) in duplicate wells.
4. Standard assay conditions for test compounds:
   Test concentration: 2 μM (DMSO≤1%);
   Replicates: n=2;
   Directions: bi-directional transport including A→B and B→A;
   Incubation time: single time point, 2 hours;
   Transport buffer: HBSS containing 10 mM HEPES, pH7.40±0.05;
   Incubation condition: 37±1° C., 5% CO2, relatively saturated humidity.
5. Spike dosing solution and mix with transport buffer and Stop Solution (containing an appropriate internal standard (IS)) as T0 sample.
6. At the end of incubation, sample solutions from both donor and receiver wells and mix with Stop Solution immediately.
7. All samples including TO samples, donor samples and receiver samples are analyzed using LC/MS/MS. Concentrations of test compound are expressed as peak area ratio of analytes versus IS without a standard curve.

### Microsome Metabolic Stability (MMS) Assay

The stability of the exemplary compounds was measured in the microsome metabolic stability assay as follows:
1) Test compounds will be incubated at 37° C. with liver microsomes (pooled from multiple donors) at 1 μM in the presence of a NADPH regenerating system at 0.5 mg/ml microsomal protein.
2) Positive controls include Testosterone (3A4 substrate), Propafenone (2D6) and Diclofenac (2C9). They will be incubated with microsomes in the presence of a NADPH regenerating system.
3) Time samples (0, 5, 15, 30, 45 and 60 minutes) will be removed, immediately mixed with cold acetonitrile containing internal standard (IS). Test compound incubated with microsomes without NADPH regenerating system for 60 min will be also included.
4) Single point for each test condition (n=1).
5) Samples will be analyzed by LC/MS/MS; disappearance of test compound will be assessed base on peak area ratios of analyte/IS(no standard curve).
6) An excel data summary, calculated intrinsic clearance and t½ values will be provided.
7) Using the following equation to calculate the microsome clearance:
   int(mic)=0.693/half life/mg microsome protein per mLwt: 40 g/kg, 30 g/kg, 32 g/kg, 20 g/kg and 88 g/kg for rat, monkey, dog, human and mouse.CLint(mic) to calculate the whole the liver clearance: microsomal protein/g liver weight: 45 mg/g for 5 speciesint(liver)=CLint(mic)*mg microsomal protein/g liver weight*g liver weight/kg body weight.

### In Vitro Metabolic Stability of Test Compounds in CD-1 Mouse, SD Rat, Beagle Dog, Cynomolgus Monkey and Human Cryopreserved Hepatocytes

1. Test compound (at 1 μM) is incubated with cryopreserved hepatocytes (0.5×106 cells per mL) in duplicates (n=2) at 37° C. using 96-well plate format.
2) Time points are 0, 15, 30, 60 and 90 minutes in separate plates and medium control samples without cells at 0 and 90 minutes are also incubated. At each time point the reaction will be stopped by adding organic solution containing internal standard (IS).
3. Positive controls 7-ethoxycoumarin and 7-hydroxycoumarin are included in parallel.
4. Samples are analyzed by LC-MS/MS. Disappearance of test compound is assessed based on peak area ratios of analyte/IS (no standard curve).

Further embodiments of the present invention are disclosed in the following numbered items.
1. A compound of formula (I):

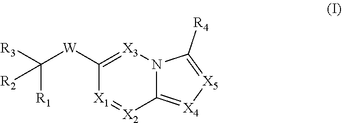

or an enantiomer, diastereoisomer, tautomer, pharmaceutically acceptable solvate, pharmaceutically acceptable crystal form, pharmaceutically acceptable salt or a prodrug thereof, wherein:
R₁ is selected from the group consisting of hydrogen, chloro, fluoro, cyano, formyl, (C₁₋₂)alkyl, (C₂)alkenyl, (C₂)alkynyl (C₁₋₂)haloalkyl, —(C₁₋₂ alkylene)-OH and —(C₁₋₂ alkylene)-O—(C₁₋₂ alkyl), preferably wherein R₁ is selected from the group consisting of hydrogen, chloro, fluoro, cyano, formyl, (C₁₋₂)alkyl, (C₂)alkenyl, (C₂)alkynyl and (C₁₋₂)haloalkyl;
R₂ and R₃ are independently each (C₁₋₂)alkyl or (C₁₋₂)haloalkyl, or R₂ and R₃ together with the carbon atom to which they are attached form cyclopropyl;
W is selected from —NHS(O)_{y}—, —S(O)_{y}NH—, —NHS(O)(NH)—, —NHS(O)(NCH₃)—, —S(O)(NH)—NH—, —S(O)(NCH₃)—NH—, wherein y is 1 or 2;
X₁ and X₃ are independently selected from the group consisting of N, CH, and CF;
X₂ is N or C—Y_{C2}—R_{C2},
   wherein Y_{C2} is selected from a covalent bond, C₁₋₅ alkylene, C₂₋₅ alkenylene, C₂₋₅ alkynylene, cycloalkylene and heterocycloalkylene wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₋₅ alkyl), —S(C₁₋₅ haloalkyl), NH₂, NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and further wherein said cycloalkylene and said heterocycloalkylene are each optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), and —(C₁₋₅ alkylene)N(C₁₋₅ alkyl)CON(C₁₋₅ alkyl)(C₁₋₅ alkyl), preferably selected from halogen, CN, OH, C₁₋₅ alkyl, O(C₁₋₅ alkyl), SH, S(C₁₋₅ alkyl), NH₂, NH(C₁₋₅ alkyl), and N(C₁₋₅ alkyl)(C₁₋₅ alkyl),
   preferably wherein Y_{C2} is selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and wherein R_{C2} is selected from hydrogen, halo, —OH, —NH₂, —SH, —CN, C₁₋₁₂ alkyl, C₂₋₁₂ alkenyl, C₂₋₁₂ alkynyl, cycloalkyl, heterocycloalkyl, aryl, and heteroaryl,
      wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), and
wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl);
   X₄ is N or C—R_{C4},
      wherein R_{C4} is selected from hydrogen, halo, C₁₋₆ alkyl, C₂₋₆ alkynyl, —O(C₁₋₅ alkyl), —S(C₁₋₆ alkyl), —NH(C₁₋₆ alkyl), —N(C₁₋₆ alkyl)(C₁₋₆ alkyl), —CO(C₁₋₆ alkyl), C₁₋₆ haloalkyl, —O(C₁₋₆ haloalkyl), —S(C₁₋₆ haloalkyl), —NH(C₁₋₆ haloalkyl), —N(C₁₋₅ haloalkyl)₂, —CO—(C₁₋₅ haloalkyl), —(C₀₋₃ alkylene)-cycloalkyl, —O—(C₀₋₃ alkylene)-cycloalkyl, —CO—(C₀₋₃ alkylene)-cycloalkyl, —(C₀₋₃ alkylene)-heterocycloalkyl, —O—(C₀₋₃ alkylene)-heterocycloalkyl, —CO—(C₀₋₃ alkylene)-heterocycloalkyl, —(C₀₋₃ alkylene)-aryl, —O—(C₀₋₃ alkylene)-aryl, —CO—(C₀₋₃ alkylene)-aryl, —(C₀₋₃ alkylene)-heteroaryl, —O—(C₀₋₃ alkylene)-heteroaryl and —CO—(C₀₋₃ alkylene)-heteroaryl,
         wherein said alkyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), preferably selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and
         wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₄ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl);
   X₅ is N or C—R_{C5},
      wherein R_{C5} is selected from hydrogen, halo, C₁₋₅ alkyl, —O(C₁₋₅ alkyl), —S(C₁₋₅ alkyl), —NH(C₁₋₆ alkyl), —N(C₁₋₅ alkyl)(C₁₋₅ alkyl) and C₁₋₅ haloalkyl;
   R₄ is Y_{R5}—R_{R5},
      wherein Y_{R5} is selected from a covalent bond, C₁₋₄ alkylene, C₂₋₄ alkenylene, and C₂₋₄ alkynylene, wherein said alkylene, said alkenylene and said alkynylene are each optionally substituted with one or more groups independently selected from halogen, CN, OH, O(C₁₋₅ alkyl), O(C₁₋₅ haloalkyl)SH, S(C₁₋₅ alkyl), S(C₁₋₅ haloalkyl)NH₂, NH(C₁₋₅ alkyl), NH(C₁₋₅ haloalkyl), N(C₁₋₅ alkyl)(C₁₋₅ alkyl), and N(C₁₋₅ haloalkyl)(C₁₋₅ alkyl) and further wherein one or more —CH₂— units comprised in said alkylene, said alkenylene or said alkynylene are each optionally replaced by a group independently selected from —O—, NH—, N(C₁₋₅ alkyl)-, CO—, —COO—, S—, —SO—, and SO₂—, and
      wherein R_{R5} is selected from C₁₋₁₂ alkyl, C₂₋₁₂ alkenyl, C₂₋₁₂ alkynyl, cycloalkyl, heterocycloalkyl, aryl, and heteroaryl,
         wherein said alkyl, alkenyl, or alkynyl is optionally substituted with one or more groups independently selected from halogen, CN, OH, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl), and wherein said cycloalkyl, heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl).
2. The compound of item 1, wherein R₁ is selected from the group consisting of cyano, (C₁₋₂)alkyl, and (C₁₋₂)haloalkyl.
3. The compound of item 1 or 2, wherein R₁ is selected from the group consisting of cyano, methyl and fluoromethyl.
4. The compound of any one of items 1 to 3, wherein R₁ is cyano.
5. The compound of any one of items 1 to 4, wherein R₂ and R₃ together with the carbon atom to which they are attached form cyclopropyl.
6. The compound of any one of items 1 to 5, wherein W is —NHS(O)₂—.
7. The compound of any one of items 1 to 6, wherein X₁ and X₃ are each CH.
8. The compound of any one of items 1 to 7, wherein X₂ is C—Y_{C2}—R_{C2}.
9. The compound of item 8, wherein —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂₋₁₂ alkenyl, —O—C₂₋₁₂ alkynyl, —NH—C₂₋₁₂ alkynyl, —N(C₁₋₅ alkyl)-C₂₋₁₂ alkynyl, —(C₀₋₃ alkylene)-cycloalkyl, —CO—(C₀₋₃ alkylene)-cycloalkyl, —(C₀₋₃ alkylene)-CO-cycloalkyl, —CONH—(C₀₋₃ alkylene)-cycloalkyl, (C₀₋₃ alkylene)-CONH-cycloalkyl, —NHCO—(C₀₋₃ alkylene)-cycloalkyl, —(C₀₋₃ alkylene)-NHCO-cycloalkyl, —NH—(C₀₋₃ alkylene)-cycloalkyl, —(C₀₋₃ alkylene)-NH-cycloalkyl, —O—(C₀₋₃ alkylene)-cycloalkyl, —(C₀₋₃ alkylene)-O-cycloalkyl, —SO₂—(C₀₋₃ alkylene)-cycloalkyl, —(C₀₋₃ alkylene)-SO₂-cycloalkyl, —CONH-cycloalkyl, —NHCO-cycloalkyl, —NH-cycloalkyl, —O-cycloalkyl, —CO-cycloalkyl, —SO₂-cycloalkyl, —(C₀₋₃ alkylene)-heterocycloalkyl, —CO—(C₀₋₃ alkylene)-heterocycloalkyl, —(C₀₋₃ alkylene)-CO-heterocycloalkyl, —CONH—(C₀₋₃ alkylene)-heterocycloalkyl, —(C₀₋₃ alkylene)-CONH-heterocycloalkyl, —NHCO—(C₀₋₃ alkylene)-heterocycloalkyl, —(C₀₋₃ alkylene)-NHCO-heterocycloalkyl, —NH—(C₀₋₃ alkylene)-heterocycloalkyl, —(C₀₋₃ alkylene)-NH-heterocycloalkyl, —O—(C₀₋₃ alkylene)-heterocycloalkyl, —(C₀₋₃ alkylene)-O-cycloalkyl, —SO₂—(C₀₋₃ alkylene)-heterocycloalkyl, —(C₀₋₃ alkylene)-SO₂-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)-aryl, —CO—(C₀₋₃ alkylene)-aryl, —(C₀₋₃ alkylene)-CO-aryl, —CONH—(C₀₋₃ alkylene)-aryl, —(C₀₋₃ alkylene)-CONH-aryl, —NHCO—(C₀₋₃ alkylene)-aryl, —(C₀₋₃ alkylene)-NHCO-aryl, —NH—(C₀₋₃ alkylene)-aryl, —(C₀₋₃ alkylene)-NH-aryl, —O—(C₀₋₃ alkylene)-aryl, —(C₀₋₃ alkylene)-O-aryl, —SO₂—(C₀₋₃ alkylene)-aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)-heteroaryl, —CO—(C₀₋₃ alkylene)-heteroaryl, —(C₀₋₃ alkylene)-CO-heteroaryl, —CONH—(C₀₋₃ alkylene)-heteroaryl, —(C₀₋₃ alkylene)-CONH-heteroaryl, —NHCO—(C₀₋₃ alkylene)-heteroaryl, —(C₀₋₃ alkylene)-NHCO-heteroaryl, —NH—(C₀₋₃ alkylene)-heteroaryl, —(C₀₋₃ alkylene)-NH-heteroaryl, —O—(C₀₋₃ alkylene)-heteroaryl, —(C₀₋₃ alkylene)-O-heteroaryl, —SO₂—(C₀₋₃ alkylene)-heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, NH(—C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —O(C₁₋₅ alkyl), —O(C₁₋₅ haloalkyl), —C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl) and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)OH—, —(C₁₋₅ alkylene)O—(C₁₋₅ alkyl), —(C₁₋₅ alkylene)-O(C₁₋₅ haloalkyl), —(C₁₋₅ alkylene)SH—, —(C₁₋₅ alkylene)S—(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)NH(—C₁₋₅ alkyl), —(C₁₋₅ alkylene)-NH(C₁₋₅ haloalkyl), —(C₁₋₅ alkylene)N(—C₁₋₅ alkyl)(C₁₋₅ alkyl), —(C₁₋₅ alkylene)N(—C₁₋₅ alkyl)(C₁₋₅ haloalkyl), —(C₁₋₅ alkylene)-(N-heterocycloalkyl), —(C₁₋₅ alkylene)N(—C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)CONH(—C₁₋₅ alkyl), —(C₁₋₅ alkylene)CON(—C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)N(—C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)NHCON(—C₁₋₅ alkyl)(C₁₋₅ alkyl), —(C₁₋₅ alkylene)N(—C₁₋₅ alkyl)CONH₂, —(C₁₋₅ alkylene)N(—C₁₋₅ alkyl)CONH—(C₁₋₅ alkyl), and —(C₁₋₅ alkylene)N(—C₁₋₅ alkyl)CON(C₁₋₅ alkyl)(C₁₋₅ alkyl), preferably selected from, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, CONH(—C₁₋₅ alkyl), and CON(—C₁₋₅ alkyl)(C₁₋₅ alkyl).
10. The compound of item 8 or 9, wherein —Y_{C2}—R_{C2} is selected from —(C₀₋₃ alkylene)-heterocycloalkyl, —CO—(C₀₋₃ alkylene)-heterocycloalkyl, —(C₀₋₃ alkylene)-CO-heterocycloalkyl, —CONH—(C₀₋₃ alkylene)-heterocycloalkyl, —(C₀₋₃ alkylene)-CONH-heterocycloalkyl, —NHCO—(C₀₋₃ alkylene)-heterocycloalkyl, —(C₀₋₃ alkylene)-NHCO-heterocycloalkyl, —NH—(C₀₋₃ alkylene)-heterocycloalkyl, —(C₀₋₃ alkylene)-NH-heterocycloalkyl, —O—(C₀₋₃ alkylene)-heterocycloalkyl, —(C₀₋₃ alkylene)-O-cycloalkyl, —SO₂—(C₀₋₃ alkylene)-heterocycloalkyl, —(C₀₋₃ alkylene)-SO₂-heterocycloalkyl, —CONH-heterocycloalkyl, —NHCO-heterocycloalkyl, —NH-heterocycloalkyl, —O-heterocycloalkyl, —CO-heterocycloalkyl, —SO₂-heterocycloalkyl, —(C₀₋₃ alkylene)-aryl, —CO—(C₀₋₃ alkylene)-aryl, —(C₀₋₃ alkylene)-CO-aryl, —CONH—(C₀₋₃ alkylene)-aryl, —(C₀₋₃ alkylene)-CONH-aryl, —NHCO—(C₀₋₃ alkylene)-aryl, —(C₀₋₃ alkylene)-NHCO-aryl, —NH—(C₀₋₃ alkylene)-aryl, —(C₀₋₃ alkylene)-NH-aryl, —O—(C₀₋₃ alkylene)-aryl, —(C₀₋₃ alkylene)-O-aryl, —SO₂—(C₀₋₃ alkylene)-aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)-heteroaryl, —CO—(C₀₋₃ alkylene)-heteroaryl, —(C₀₋₃ alkylene)-CO-heteroaryl, —CONH—(C₀₋₃ alkylene)-heteroaryl, —(C₀₋₃ alkylene)-CONH-heteroaryl, —NHCO—(C₀₋₃ alkylene)-heteroaryl, —(C₀₋₃ alkylene)-NHCO-heteroaryl, —NH—(C₀₋₃ alkylene)-heteroaryl, —(C₀₋₃ alkylene)-NH-heteroaryl, —O—(C₀₋₃ alkylene)-heteroaryl, —(C₀₋₃ alkylene)-O-heteroaryl, —SO₂—(C₀₋₃ alkylene)-heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).
11. The compound of any one of items 8 to 10, wherein —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)aryl, —CONH-aryl, —NHCO-aryl, —NH-aryl, —O-aryl, —CO-aryl, —SO₂-aryl, —(C₀₋₃ alkylene)-heteroaryl, —CONH-heteroaryl, —NHCO-heteroaryl, —NH-heteroaryl, —O-heteroaryl, —CO-heteroaryl and —SO₂-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).
12. The compound of any one of claims 8 to 11, wherein —Y_{C2}—R_{C2} is selected from —(C₀₋₃ alkylene)-heterocycloalkyl, —(C₀₋₃ alkylene)-aryl, and —(C₀₋₃ alkylene)-heteroaryl, wherein said heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), —CO(C₁₋₅ alkyl), CONH₂, CONH(C₁₋₅ alkyl), and CON(C₁₋₅ alkyl)(C₁₋₅ alkyl).
13. The compound of any one of items 8 to 12, wherein —Y_{C2}—R_{C2} is selected from heterocycloalkyl, aryl, and heteroaryl, preferably heterocycloalkyl and heteroaryl, more preferably heterocycloalkyl, wherein said heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ haloalkyl), S(C₁₋₅ alkyl), NH₂, NH(C₁₋₅ alkyl), NH(C₁₋₅ haloalkyl), N(C₁₋₅ alkyl)(C₁₋₅ alkyl), —N(C₁₋₅ haloalkyl)(C₁₋₅ alkyl), —CO(C₁₋₅ alkyl), CONH₂, CONH(C₁₋₅ alkyl), and CON(C₁₋₅ alkyl)(C₁₋₅ alkyl).
14. The compound of any one of items 8 to 13, wherein —Y_{C2}—R_{C2} is optionally substituted aryl, preferably —Y_{C2}—R_{C2} is phenyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).
15. The compound of any one of items 8 to 13, wherein —Y_{C2}—R_{C2} is an optionally substituted heteroaryl, preferably wherein —Y_{C2}—R_{C2} is imidazolyl, pyridazinyl, thiazolyl, pyridinyl, pyrimidinyl, pyrazinyl, or indazolyl, wherein heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).
16. The compound of any one of items 8 to 13, wherein —Y_{C2}—R_{C2} is optionally substituted heterocycloalkyl, preferably wherein —Y_{C2}—R_{C2} is morpholinyl, 1,1-dioxothiomorpholinyl, azetinyl, pyrrolidinyl, piperidinyl, 6-oxo-1,6-dihydropyridinyl, or piperazinyl, wherein heterocycloalkyl is optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), —N(C₁₋₅ haloalkyl)(C₁₋₅ alkyl), —CO(C₁₋₅ alkyl), CONH₂, CONH(C₁₋₅ alkyl), and CON(C₁₋₅ alkyl)(C₁₋₅ alkyl).
17. The compound of item 16, wherein —Y_{C2}—R_{C2} is piperazinyl, optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), —N(C₁₋₅ haloalkyl)(C₁₋₅ alkyl), —CO(C₁₋₅ alkyl), CONH₂, CONH(C₁₋₅ alkyl), and CON(C₁₋₅ alkyl)(C₁₋₅ alkyl), preferably wherein —Y_{C2}—R_{C2} is piperazinyl (preferably N-piperazinyl) optionally substituted (preferably N-substituted) with C₀(C₁₋₅ alkyl), CONH₂, CONH(C₁₋₅ alkyl), and CON(C₁₋₅ alkyl)(C₁₋₅ alkyl), more preferably wherein —Y_{C2}—R_{C2} is piperazinyl (preferably N-piperazinyl) substituted (preferably N-substituted, preferably at a different N-atom than that attached to the ring system as shown in formula (I)), with CON(C₁₋₅ alkyl)(C₁₋₅ alkyl), preferably with —CON(CH₃)₂.
18. The compound of item 16, wherein —Y_{C2}—R_{C2} is 2-oxaspiro[3.5]non-6-en-7-yl, 2-oxaspiro[3.5]non-7-yl, 2-oxa-8-azaspiro[4.5]dec-8-yl, 9-oxa-3-azaspiro[5.5]undec-3-yl, 2-oxa-6-azaspiro[3.4]oct-6-yl, 1-oxa-7-azaspiro[3.5]non-7-yl, 1-oxa-8-azaspiro[4.5]dec-8-yl, 6-oxa-2-azaspiro[3.3]hept-2-yl, 2,8-diazaspiro[4.5]dec-8-yl, 7-oxa-3-azabicyclo[3.3.0]oct-3-yl, 8-oxa-3-azabicyclo[4.3.0]non-3-yl, 2-oxa-6-azaspiro[3.5]non-6-yl, 7-oxo-3,6,8-triazabicyclo[4.3.0]non-3-yl, 3-pyrrolino[3,4-c]pyrazol-2-yl, 3,6-diazabicyclo[3.1.1]hept-3-yl, or 2,7-diazaspiro[3.5]non-7-yl.
19. The compound of any one of items 1 to 16, wherein X₄ is C—R_{C4}, wherein R_{C4} is selected from hydrogen, halo, C₁₋₆ alkyl, C₂₋₆ alkynyl, —O—C₁₋₆ alkyl, —S—C₁₋₅ alkyl, —NH—C₁₋₆ alkyl, C₁₋₆ haloalkyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl is optionally substituted with one or more groups independently selected from halogen, CN, OH, O(C₁₋₅ alkyl), O(C₁₋₅ haloalkyl), SH, S(C₁₋₅ alkyl), O(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl), and wherein said cycloalkyl, heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), O(C₁₋₅ haloalkyl), SH, S(C₁₋₅ haloalkyl), S(C₁₋₅ alkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl).
20. The compound of item 19, wherein R_{C4} is selected from hydrogen, halo, C₁₋₆ alkyl, C₂₋₆ alkynyl, —O—C₁₋₆ alkyl, —S—C₁₋₆ alkyl, —NH—C₁₋₆ alkyl, and C₁₋₆ haloalkyl, preferably wherein R_{C4} is selected from hydrogen, halo, C₁₋₂ alkyl, and C₂₋₃ alkynyl, more preferably wherein R_{C4} is selected from hydrogen, halo, and C₁₋₂ alkyl, even more preferably wherein R_{C4} is hydrogen or halo.
21. The compound of item 19, wherein R_{C4} is selected from —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
22. The compound of item 19 or 21, wherein R_{C4} is selected from —(C₀₋₃ alkylene)-cycloalkyl, —(C₀₋₃ alkylene)-heterocycloalkyl, and —(C₀₋₃ alkylene)-heteroaryl, preferably wherein R_{C4} is selected from cycloalkyl, heterocycloalkyl, and heteroaryl, wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —NH(C₁₋₅ haloalkyl)(C₁₋₅ alkyl), —CONH₂, —CONH(C₁₋₅ alkyl), and —CON(C₁₋₅ alkyl)(C₁₋₅ alkyl).
23. The compound of item 21, wherein R_{C4} is selected from heterocycloalkyl and heteroaryl, wherein said heterocycloalkyl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —NH(C₁₋₅ haloalkyl)(C₁₋₅ alkyl), —CONH₂, —CONH(C₁₋₅ alkyl), and —CON(C₁₋₅ alkyl)(C₁₋₅ alkyl).
24. The compound of item 19, wherein R_{C4} is selected from —CH₂-cycloalkyl, —CH₂-heterocycloalkyl, —CH₂-aryl and —CH₂-heteroaryl, more preferably wherein R_{C4} is selected from —CH₂-heterocycloalkyl, and —CH₂-heteroaryl, wherein said cycloalkyl, heterocycloalkyl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
25. The compound of item 23, wherein R_{C4} is heteroaryl, preferably wherein R_{C4} is imidazolyl, pyridazinyl, thiazolyl, pyridinyl, pyrimidinyl, pyrazinyl, or indazolyl, wherein said heteroaryl may be optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
26. The compound of item 23, wherein R_{C4} is heterocycloalkyl, preferably wherein R_{C4} is morpholinyl, 1,1-dioxothiomorpholinyl, azetinyl, pyrrolidinyl, piperidinyl, 6-oxo-1,6-dihydropyridinyl, or piperazinyl, wherein said heterocycloalkyl is optionally substituted with one or more groups independently selected from halogen, —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CONH₂, —CONH(C₁₋₅ alkyl), and —CON(C₁₋₅ alkyl)(C₁₋₅ alkyl).
27. The compound of item 26, wherein R_{C4} is piperazinyl, preferably wherein R_{C4} is piperazinyl (preferably N-piperazinyl) substituted (preferably N-substituted, preferably at a different N-atom than that attached to the ring system shown in formula (I)), with —CON(C₁₋₅ alkyl)(C₁₋₅ alkyl), preferably with —CON(CH₃)₂.
28. The compound of item 23, wherein R_{C4} is heterocycloalkyl, preferably wherein R_{C4} is 2-oxaspiro[3.5]non-6-en-7-yl, 2-oxaspiro[3.5]non-7-yl, 2-oxa-8-azaspiro[4.5]dec-8-yl, 9-oxa-3-azaspiro[5.5]undec-3-yl, 2-oxa-6-azaspiro[3.4]oct-6-yl, 1-oxa-7-azaspiro[3.5]non-7-yl, 1-oxa-8-azaspiro[4.5]dec-8-yl, 6-oxa-2-azaspiro[3.3]hept-2-yl, 2,8-diazaspiro[4.5]dec-8-yl, 7-oxa-3-azabicyclo[3.3.0]oct-3-yl, 8-oxa-3-azabicyclo[4.3.0]non-3-yl, 2-oxa-6-azaspiro[3.5]non-6-yl, 7-oxo-3,6,8-triazabicyclo[4.3.0]non-3-yl, 3-pyrrolino[3,4-c]pyrazol-2-yl, 3,6-diazabicyclo[3.1.1]hept-3-yl, or 2,7-diazaspiro[3.5]non-7-yl.
29. The compound of any one of items 1 to 28, wherein if X₂ comprises cycloalkyl, heterocycloalkyl, aryl or heteroaryl, X₄ is C—R_{C4} wherein R_{C4} is selected from hydrogen, halo, C₁₋₆ alkyl, —O—C₁₋₆ alkyl, —S—C₁₋₆ alkyl, —NH—C₁₋₅ alkyl, and C₁₋₆ haloalkyl; preferably wherein R_{C4} is selected from hydrogen, and halo.
30. The compound of any one of items 1 to 28, wherein if X₄ comprises cycloalkyl, heterocycloalkyl, aryl or heteroaryl, X₂ does not comprise any of the groups selected from cycloalkyl, heterocycloalkyl, aryl and heteroaryl.
31. The compound of any one of items 1 to 28, wherein if X₂ comprises cycloalkyl, heterocycloalkyl, aryl or heteroaryl and X₄ comprises cycloalkyl, heterocycloalkyl, aryl or heteroaryl, then together R_{C4} and —Y_{C2}—R_{C2} include not more than 12 non-hydrogen atoms, preferably not more than 10 non-hydrogen atoms.
32. The compound of any one of items 1 to 31, wherein X₅ is C—R_{C5}, wherein R_{C5} is selected from hydrogen, halo, C₁₋₃ alkyl, —O—C₁₋₃ alkyl, —S—C₁₋₃ alkyl, —NH—C₁₋₃ alkyl, and C₁₋₃ haloalkyl, preferably, wherein R_{C5} is selected from hydrogen, halo, C₁₋₃ alkyl, and C₁₋₃ haloalkyl.
33. The compound of any one of items 1 to 32, wherein Y_{R5} is selected from a covalent bond, C₁₋₂ alkylene, —CO—(C₁₋₂ alkylene), —(C₁₋₂ alkylene)-CO—, —CONH—(C₁₋₂ alkylene)-, —(C₁₋₂ alkylene)-CONH—, —NHCO—(C₁₋₂ alkylene)-, —(C₁₋₂ alkylene)-NHCO—, —NH—(C₁₋₂ alkylene)-, —(C₁₋₂ alkylene)-NH—, —O—(C₁₋₂ alkylene)-, —(C₁₋₂ alkylene)-O—, SO₂—(C₁₋₂ alkylene)-, —(C₁₋₂ alkylene)SO₂—, —CONH—, CON(C₁₋₅ alkyl)-, —NHCO—, —N(C₁₋₅ alkyl)CO—, —NH—, —O—, —CO—, —COO— and SO₂—.
34. The compound of any one of items 1 to 33, wherein R_{R5} is selected from cycloalkyl, heterocycloalkyl, aryl, and heteroaryl, preferably wherein R_{R5} is selected from heterocycloalkyl, aryl, and heteroaryl, more preferably wherein R_{R5} is selected from aryl and heteroaryl, most preferably wherein R_{R5} is heteroaryl wherein said cycloalkyl, heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl).
35. The compound of any one of items 1 to 34, wherein Y_{R5} is selected from a covalent bond, C₁₋₂ alkylene, —CO—(C₁₋₂ alkylene)-, —(C₁₋₂ alkylene)-CO—, —CONH—(C₁₋₂ alkylene)-, —(C₁₋₂ alkylene)-CONH—, —NHCO—(C₁₋₂ alkylene), —(C₁₋₂ alkylene)-NHCO—, —NH—(C₁₋₂ alkylene), —(C₁₋₂ alkylene)-NH—, —O—(C₁₋₂ alkylene), —(C₁₋₂ alkylene)-O—, —SO₂—(C₁₋₂ alkylene)-, —(C₁₋₂ alkylene)-SO₂—, —CONH—, —NHCO—, —NH—, —O—, —CO— and —SO₂—.
36. The compound of item 34 or 35, wherein R₄ is selected from —(C₀₋₂ alkylene)-cycloalkyl, —CO—(C₀₋₂ alkylene)-cycloalkyl, —(C₀₋₂ alkylene)-CO-cycloalkyl, —CONH—(C₀₋₂ alkylene)-cycloalkyl, —(C₀₋₂ alkylene)-CONH-cycloalkyl, —NHCO—(C₀₋₂ alkylene)-cycloalkyl, —(C₀₋₂ alkylene)-NHCO-cycloalkyl, —NH—(C₀₋₂ alkylene)-cycloalkyl, —(C₀₋₂ alkylene)-NH-cycloalkyl, —O—(C₀₋₂ alkylene)-cycloalkyl, —(C₀₋₂ alkylene)-O-cycloalkyl, —SO₂—(C₀₋₂ alkylene)-cycloalkyl, —(C₀₋₂ alkylene)-SO₂-cycloalkyl, —CONH— cycloalkyl, —NHCO-cycloalkyl, —NH-cycloalkyl, —O-cycloalkyl, —CO-cycloalkyl, —SO₂-cycloalkyl, —(C₀₋₂ alkylene)-heterocycloalkyl, —CO—(C₀₋₂ alkylene)-heterocycloalkyl, —(C₀₋₂ alkylene)-CO-heterocycloalkyl, —CONH—(C₀₋₂ alkylene)-heterocycloalkyl, —(C₀₋₂ alkylene)-CONH-heterocycloalkyl, —NHCO—(C₀₋₂ alkylene)-heterocycloalkyl, —(C₀₋₂ alkylene)-NHCO-heterocycloalkyl, —NH—(C₀₋₂ alkylene)-heterocycloalkyl, —(C₀₋₂ alkylene)-NH-heterocycloalkyl, —O—(C₀₋₂ alkylene)-heterocycloalkyl, —(C₀₋₂ alkylene)-O-heterocycloalkyl, —SO₂—(C₀₋₂ alkylene)-heterocycloalkyl, —(C₀₋₂ alkylene)-SO₂-heterocycloalkyl, —CONH-heterocycloalkyl, —NHCO-heterocycloalkyl, —NH— heterocycloalkyl, —O-heterocycloalkyl, —CO-heterocycloalkyl, —SO₂-heterocycloalkyl, —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, —SO₂—(C₀₋₂ alkylene)-aryl, —(C₀₋₂ alkylene)-SO₂-aryl, —CONH-aryl, —NHCO-aryl, —NH-aryl, —O-aryl, —CO-aryl, —SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)heteroaryl, —(C₀₋₂ alkylene)-NH-heteroaryl, —O—(C₀₋₂ alkylene)heteroaryl, —(C₀₋₂ alkylene)-O-heteroaryl, —SO₂—(C₀₋₂ alkylene)heteroaryl, —(C₀₋₂ alkylene)-SO₂-heteroaryl, —CONH-heteroaryl, —NHCO-heteroaryl, —NH-heteroaryl, —O-heteroaryl, —CO-heteroaryl, and —SO₂-heteroaryl, wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
37. The compound of item 36, wherein R₄ is selected from —(C₀₋₂ alkylene)aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, —SO₂—(C₀₋₂ alkylene)-aryl, —(C₀₋₂ alkylene)-SO₂-aryl, —CONH-aryl, —NHCO-aryl, —NH-aryl, —O-aryl, —CO-aryl, —SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, —SO₂—(C₀₋₂ alkylene)-heteroaryl, —(C₀₋₂ alkylene)-SO₂-heteroaryl, —CONH-heteroaryl, —NHCO-heteroaryl, —NH-heteroaryl, —O-heteroaryl, —CO-heteroaryl, and —SO₂-heteroaryl, wherein said aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
38. The compound of any one of items 1 to 32, wherein R₄ is selected from C₁₋₁₂ alkyl, C₂₋₁₂ alkenyl, C₂₋₁₂ alkynyl, cycloalkyl, heterocycloalkyl, aryl, and heteroaryl, preferably wherein R₄ is selected from cycloalkyl, heterocycloalkyl, aryl, and heteroaryl, more preferably wherein R₄ is selected from aryl, and heteroaryl, even more preferably wherein R₄ is heteroaryl, wherein said alkyl, alkenyl, or alkynyl is optionally substituted with one or more groups independently selected from halogen, CN, OH, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl), and wherein said cycloalkyl, heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl).
39. The compound of item 37 or 38, wherein R₄ is a five membered heteroaryl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
40. The compound of item 39, wherein the five membered heteroaryl is selected from imidazolyl, isoxazolyl, pyrazolyl, 1,2,3-triazolyl, 1,2,4-triazolyl, thiazolyl, 1,2,4-oxadiazolyl, 1,3,4-oxadiazolyl, 1,2,4-thiadiazolyl, or 1,3,4-thiadiazolyl, preferably wherein the five membered heteroaryl is 1,2,4-thiadiazolyl, optionally substituted with one or more groups independently selected from halogen, —CN, —OH, C₁₋₅ alkyl, C₁₋₅ haloalkyl, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CONH₂, —CONH(C₁₋₅ alkyl), and —CON(C₁₋₅ alkyl)(C₁₋₅ alkyl), preferably optionally substituted with C₁₋₅ alkyl, C₁₋₅ haloalkyl, —O(C₁₋₅ alkyl), —SH, —S(C₁₋₅ alkyl), more preferably optionally substituted with C₁₋₅ alkyl, C₁₋₅ haloalkyl, even more preferably optionally substituted with C₁₋₅ haloalkyl, preferably selected from —CH₂F, —CHF₂ and CF₃, most preferably optionally substituted with —CHF₂.
41. A pharmaceutical composition comprising the compound of any one of items 1 to 40 or a pharmaceutically acceptable salt, hydrate or solvate thereof, and a pharmaceutically acceptable carrier.
42. The compound of any one of items 1 to 40 or a pharmaceutically acceptable salt, hydrate or solvate thereof, or a pharmaceutical composition of item 41, for use in therapy.
43. The compound for use or the pharmaceutical composition for use of item 42, for use in a method of treating a disease or disorder in which PARG activity is implicated.
44. The compound for use or the pharmaceutical composition for use of item 42, for use in a method of treating a proliferative disorder.
45. The compound for use of the pharmaceutical composition for use of item 44, wherein the proliferative disorder is cancer, preferably a human cancer.

Further embodiments of the present invention are disclosed in the following numbered paragraphs.
1. A compound of formula (I):

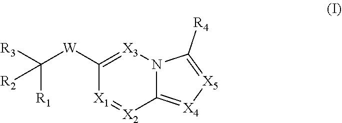

or an enantiomer, diastereoisomer, tautomer, pharmaceutically acceptable solvate, pharmaceutically acceptable crystal form, pharmaceutically acceptable salt or a prodrug thereof, wherein:
R₁ is selected from the group consisting of hydrogen, chloro, fluoro, cyano, formyl, (C₁₋₂)alkyl, (C₂)alkenyl, (C₂)alkynyl and (C₁₋₂)haloalkyl;
R₂ and R₃ are independently each (C₁₋₂)alkyl or (C₁₋₂)haloalkyl, or R₂ and R₃ together with the carbon atom to which they are attached form cyclopropyl;
W is selected from —NHS(O)_{y}—, —S(O)_{y}NH—, —NHS(O)(NH)—, —NHS(O)(NCH₃)—, —S(O)(NH)—NH—, —S(O)(NCH₃)—NH—, wherein y is 1 or 2;
X₁ and X₃ are independently selected from the group consisting of N, CH, and CF;
X₂ is N or C—Y_{C2}—R_{C2},
   wherein Y_{C2} is selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and
   wherein R_{C2} is selected from hydrogen, halo, —OH, —NH₂, —SH, —CN, C₁₋₁₂ alkyl, C₂₋₁₂ alkenyl, C₂₋₁₂ alkynyl, cycloalkyl, heterocycloalkyl, aryl, and heteroaryl,
      wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), and
      wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl);
X₄ is N or C—R_{C4},
   wherein R_{C4} is selected from hydrogen, halo, C₁₋₆ alkyl, C₂₋₆ alkynyl, —O(C₁₋₆ alkyl), —S(C₁₋₆ alkyl), —NH(C₁₋₆ alkyl), —N(C₁₋₆ alkyl)(C₁₋₆ alkyl), —CO(C₁₋₆ alkyl), C₁₋₆ haloalkyl, —O(C₁₋₆ haloalkyl), —S(C₁₋₆ haloalkyl), —NH(C₁₋₆ haloalkyl), —N(C₁₋₅ haloalkyl)₂, —CO—(C₁₋₆ haloalkyl), —(C₀₋₃ alkylene)-cycloalkyl, —O—(C₀₋₃ alkylene)-cycloalkyl, —CO—(C₀₋₃ alkylene)-cycloalkyl, —(C₀₋₃ alkylene)-heterocycloalkyl, —O—(C₀₋₃ alkylene)-heterocycloalkyl, —CO—(C₀₋₃ alkylene)-heterocycloalkyl, —(C₀₋₃ alkylene)-aryl, —O—(C₀₋₃ alkylene)-aryl, —CO—(C₀₋₃ alkylene)-aryl, —(C₀₋₃ alkylene)-heteroaryl, —O—(C₀₋₃ alkylene)-heteroaryl and —CO—(C₀₋₃ alkylene)-heteroaryl,
      wherein said alkyl is optionally substituted with one or more groups independently selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and
      wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl);
X₅ is N or C—R_{C5},
   wherein R_{C5} is selected from hydrogen, halo, C₁₋₅ alkyl, —O(C₁₋₅ alkyl), —S(C₁₋₅ alkyl), —NH(C₁₋₆ alkyl), —N(C₁₋₆ alkyl)(C₁₋₅ alkyl) and C₁₋₅ haloalkyl;
R₄ is Y_{R5}—R_{R5},
   wherein Y_{R5} is selected from a covalent bond, C₁₋₄ alkylene, C₂₋₄ alkenylene, and C₂₋₄ alkynylene, wherein said alkylene, said alkenylene and said alkynylene are each optionally substituted with one or more groups independently selected from halogen, CN, OH, O(C₁₋₅ alkyl), O(C₁₋₅ haloalkyl)SH, S(C₁₋₅ alkyl), S(C₁₋₅ haloalkyl)NH₂, NH(C₁₋₅ alkyl), NH(C₁₋₅ haloalkyl), N(C₁₋₅ alkyl)(C₁₋₅ alkyl), and N(C₁₋₅ haloalkyl)(C₁₋₅ alkyl) and further wherein one or more —CH₂— units comprised in said alkylene, said alkenylene or said alkynylene are each optionally replaced by a group independently selected from —O—, NH—, N(C₁₋₅ alkyl)-, CO—, —COO—, S—, —SO—, and SO₂—, and
   wherein R_{R5} is selected from C₁₋₁₂ alkyl, C₁₋₁₂ alkenyl, C₂₋₁₂ alkynyl, cycloalkyl, heterocycloalkyl, aryl, and heteroaryl,
      wherein said alkyl, alkenyl, or alkynyl is optionally substituted with one or more groups independently selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).
2. The compound of paragraph 1, wherein R₁ is cyano.
3. The compound of paragraph 1 or 2, wherein R₂ and R₃ together with the carbon atom to which they are attached form cyclopropyl.
4. The compound of any one of paragraphs 1 to 3, wherein W is —NHS(O)₂—.
5. The compound of any one of paragraphs 1 to 4, wherein X₁ and X₃ are each CH.
6. The compound of any one of paragraphs 1 to 5, wherein X₂ is C—Y_{C2}—R_{C2}, wherein —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂₋₁₂ alkenyl, —O—C₂₋₁₂ alkynyl, —NH—C₂₋₁₂ alkynyl, —N(C₁₋₅ alkyl)-C₂₋₁₂ alkynyl, —(C₀₋₃ alkylene)-cycloalkyl, —CO—(C₀₋₃ alkylene)-cycloalkyl, —(C₀₋₃ alkylene)-CO-cycloalkyl, —CONH—(C₀₋₃ alkylene)-cycloalkyl, (C₀₋₃ alkylene)-CONH-cycloalkyl, —NHCO—(C₀₋₃ alkylene)-cycloalkyl, —(C₀₋₃ alkylene)-NHCO-cycloalkyl, —NH—(C₀₋₃ alkylene)-cycloalkyl, —(C₀₋₃ alkylene)-NH-cycloalkyl, —O—(C₀₋₃ alkylene)-cycloalkyl, —(C₀₋₃ alkylene)-O-cycloalkyl, SO₂—(C₀₋₃ alkylene)-cycloalkyl, —(C₀₋₃ alkylene)SO₂-cycloalkyl, —CONH-cycloalkyl, —NHCO-cycloalkyl, —NH-cycloalkyl, —O-cycloalkyl, —CO— cycloalkyl, SO₂-cycloalkyl, —(C₀₋₃ alkylene)-heterocycloalkyl, —CO—(C₀₋₃ alkylene)-heterocycloalkyl, —(C₀₋₃ alkylene)-CO-heterocycloalkyl, —CONH—(C₀₋₃ alkylene)-heterocycloalkyl, —(C₀₋₃ alkylene)-CONH-heterocycloalkyl, —NHCO—(C₀₋₃ alkylene)-heterocycloalkyl, —(C₀₋₃ alkylene)-NHCO-heterocycloalkyl, —NH—(C₀₋₃ alkylene)-heterocycloalkyl, —(C₀₋₃ alkylene)-NH-heterocycloalkyl, —O—(C₀₋₃ alkylene)-heterocycloalkyl, —(C₀₋₃ alkylene)-O-cycloalkyl, SO₂—(C₀₋₃ alkylene)-heterocycloalkyl, —(C₀₋₃ alkylene)SO₂-heterocycloalkyl, —CONH-heterocycloalkyl, —NHCO-heterocycloalkyl, —NH— heterocycloalkyl, —O-heterocycloalkyl, —CO-heterocycloalkyl, SO₂-heterocycloalkyl, —(C₀₋₃ alkylene)-aryl, —CO—(C₀₋₃ alkylene)-aryl, —(C₀₋₃ alkylene)-CO-aryl, —CONH—(C₀₋₃ alkylene)-aryl, —(C₀₋₃ alkylene)-CONH-aryl, —NHCO—(C₀₋₃ alkylene)-aryl, —(C₀₋₃ alkylene)-NHCO-aryl, —NH—(C₀₋₃ alkylene)-aryl, —(C₀₋₃ alkylene)-NH-aryl, —O—(C₀₋₃ alkylene)-aryl, —(C₀₋₃ alkylene)-O-aryl, SO₂—(C₀₋₃ alkylene)-aryl, —(C₀₋₃ alkylene)SO₂-aryl, —CONH-aryl, —NHCO-aryl, —NH-aryl, —O-aryl, —CO-aryl, SO₂-aryl, —(C₀₋₃ alkylene)-heteroaryl, —CO—(C₀₋₃ alkylene)-heteroaryl, —(C₀₋₃ alkylene)-CO-heteroaryl, —CONH—(C₀₋₃ alkylene)-heteroaryl, —(C₀₋₃ alkylene)-CONH-heteroaryl, —NHCO—(C₀₋₃ alkylene)-heteroaryl, —(C₀₋₃ alkylene)-NHCO-heteroaryl, —NH—(C₀₋₃ alkylene)-heteroaryl, —(C₀₋₃ alkylene)-NH-heteroaryl, —O—(C₀₋₃ alkylene)-heteroaryl, —(C₀₋₃ alkylene)-O-heteroaryl, SO₂—(C₀₋₃ alkylene)-heteroaryl, —(C₀₋₃ alkylene)SO₂-heteroaryl, —CONH-heteroaryl, —NHCO-heteroaryl, —NH-heteroaryl, —O-heteroaryl, —CO-heteroaryl and SO₂-heteroaryl, wherein said alkyl, alkenyl, or alkynyl is optionally substituted with one or more groups independently selected from halogen, CN, OH, O(C₁₋₅ alkyl), O(C₁₋₅ haloalkyl), —C₁₋₅ haloalkyl, SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), —CO(C₁₋₅ alkyl), CONH₂, CONH(C₁₋₅ alkyl), and CON(C₁₋₅ alkyl)(C₁₋₅ alkyl) and wherein said cycloalkyl, heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), —CO(C₁₋₅ alkyl), CONH₂, CONH(C₁₋₅ alkyl), and CON(C₁₋₅ alkyl)(C₁₋₅ alkyl),
   preferably wherein —Y_{C2}—R_{C2} is selected from —(C₀₋₃ alkylene)-heterocycloalkyl, —CO—(C₀₋₃ alkylene)-heterocycloalkyl, —(C₀₋₃ alkylene)-CO-heterocycloalkyl, —CONH—(C₀₋₃ alkylene)-heterocycloalkyl, —(C₀₋₃ alkylene)-CONH-heterocycloalkyl, —NHCO—(C₀₋₃ alkylene)-heterocycloalkyl, —(C₀₋₃ alkylene)-NHCO-heterocycloalkyl, —NH—(C₀₋₃ alkylene)-heterocycloalkyl, —(C₀₋₃ alkylene)-NH-heterocycloalkyl, —O—(C₀₋₃ alkylene)-heterocycloalkyl, —(C₀₋₃ alkylene)-O-cycloalkyl, —SO₂—(C₀₋₃ alkylene)-heterocycloalkyl, —(C₀₋₃ alkylene)-SO₂-heterocycloalkyl, —CONH-heterocycloalkyl, —NHCO-heterocycloalkyl, —NH-heterocycloalkyl, —O-heterocycloalkyl, —CO-heterocycloalkyl, —SO₂-heterocycloalkyl, —(C₀₋₃ alkylene)-aryl, —CO—(C₀₋₃ alkylene)-aryl, —(C₀₋₃ alkylene)-CO-aryl, —CONH—(C₀₋₃ alkylene)-aryl, —(C₀₋₃ alkylene)-CONH-aryl, —NHCO—(C₀₋₃ alkylene)-aryl, —(C₀₋₃ alkylene)-NHCO-aryl, —NH—(C₀₋₃ alkylene)-aryl, —(C₀₋₃ alkylene)-NH-aryl, —O—(C₀₋₃ alkylene)-aryl, —(C₀₋₃ alkylene)-O-aryl, —SO₂—(C₀₋₃ alkylene)-aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)-heteroaryl, —CO—(C₀₋₃ alkylene)-heteroaryl, —(C₀₋₃ alkylene)-CO-heteroaryl, —CONH—(C₀₋₃ alkylene)-heteroaryl, —(C₀₋₃ alkylene)-CONH-heteroaryl, —NHCO—(C₀₋₃ alkylene)-heteroaryl, —(C₀₋₃ alkylene)-NHCO-heteroaryl, —NH—(C₀₋₃ alkylene)-heteroaryl, —(C₀₋₃ alkylene)-NH-heteroaryl, —O—(C₀₋₃ alkylene)-heteroaryl, —(C₀₋₃ alkylene)-O-heteroaryl, —SO₂—(C₀₋₃ alkylene)-heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl),
   more preferably wherein —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O-heterocycloalkyl, —CO— heterocycloalkyl, —SO₂-heterocycloalkyl, —(C₀₋₃ alkylene)aryl, —CONH-aryl, —NHCO-aryl, —NH-aryl, —O-aryl, —CO-aryl, —SO₂-aryl, —(C₀₋₃ alkylene)-heteroaryl, —CONH-heteroaryl, —NHCO-heteroaryl, —NH— heteroaryl, —O-heteroaryl, —CO-heteroaryl and —SO₂-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl),
   even more preferably wherein —Y_{C2}—R_{C2} is selected from —(C₀₋₃ alkylene)-heterocycloalkyl, —(C₀₋₃ alkylene)-aryl, and —(C₀₋₃ alkylene)-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), even more preferably wherein —Y_{C2}—R_{C2} is selected from heterocycloalkyl, aryl, and heteroaryl,
   preferably heterocycloalkyl and heteroaryl, more preferably heterocycloalkyl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ haloalkyl), —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).
7. The compound of paragraph 6, wherein —Y_{C2}—R_{C2} is optionally substituted aryl, preferably —Y_{C2}—R_{C2} is phenyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), or wherein —Y_{C2}—R_{C2} is an optionally substituted heteroaryl, preferably wherein —Y_{C2}—R_{C2} is imidazolyl, pyridazinyl, thiazolyl, pyridinyl, pyrimidinyl, pyrazinyl, or indazolyl, wherein heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), or wherein —Y_{C2}—R_{C2} is optionally substituted heterocycloalkyl, preferably wherein —Y_{C2}—R_{C2} is morpholinyl, 1,1-dioxothiomorpholinyl, azetinyl, pyrrolidinyl, piperidinyl, 6-oxo-1,6-dihydropyridinyl, or piperazinyl, wherein heterocycloalkyl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).
8. The compound of paragraph 6 or 7, wherein —Y_{C2}—R_{C2} is piperazinyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), preferably wherein —Y_{C2}—R_{C2} is piperazinyl (preferably N-piperazinyl) optionally substituted (preferably N-substituted) with CO(C₁₋₅ alkyl), —CONH₂, —CONH(C₁₋₅ alkyl), and —CON(C₁₋₅ alkyl)(C₁₋₅ alkyl), more preferably wherein —Y_{C2}—R_{C2} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.
9. The compound of any one of paragraphs 1 to 8, wherein X₄ is C—R_{C4}, wherein R_{C4} is selected from hydrogen, halo, C₁₋₆ alkyl, C₂₋₆ alkynyl, —O—C₁₋₆ alkyl, —S—C₁₋₆ alkyl, —NH—C₁₋₆ alkyl, C₁₋₆ haloalkyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl is optionally substituted with one or more groups independently selected from halogen, —CN, —OH, —O(C₁₋₅ alkyl), —O(C₁₋₅ haloalkyl), —SH, —S(C₁₋₅ alkyl), —O(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ haloalkyl), —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl),
   preferably wherein R_{C4} is selected from hydrogen, halo, C₁₋₅ alkyl, C₂₋₆ alkynyl, —O—C₁₋₆ alkyl, —S—C₁₋₆ alkyl, —NH—C₁₋₆ alkyl, and C₁₋₅ haloalkyl, preferably wherein R_{C4} is selected from hydrogen, halo, C₁₋₂ alkyl, and C₂₋₃ alkynyl, more preferably wherein R_{C4} is selected from hydrogen, halo, and C₁₋₂ alkyl, even more preferably wherein R_{C4} is hydrogen or halo.
11. The compound of any one of paragraphs 1 to 10, wherein X₅ is C—R_{C5}, wherein R_{C5} is selected from hydrogen, halo, C₁₋₃ alkyl, —O—C₁₋₃ alkyl, —S—C₁₋₃ alkyl, —NH—C₁₋₃ alkyl, and C₁₋₃ haloalkyl, preferably, wherein R_{C5} is selected from hydrogen, halo, C₁₋₃ alkyl, and C₁₋₃ haloalkyl.
12. The compound of any one of paragraphs 1 to 11, wherein R₄ is selected from —(C₀₋₂ alkylene)-cycloalkyl, —CO—(C₀₋₂ alkylene)-cycloalkyl, —(C₀₋₂ alkylene)-CO-cycloalkyl, —CONH—(C₀₋₂ alkylene)-cycloalkyl, —(C₀₋₂ alkylene)-CONH-cycloalkyl, —NHCO—(C₀₋₂ alkylene)-cycloalkyl, —(C₀₋₂ alkylene)-NHCO-cycloalkyl, —NH—(C₀₋₂ alkylene)-cycloalkyl, —(C₀₋₂ alkylene)-NH-cycloalkyl, —O—(C₀₋₂ alkylene)-cycloalkyl, —(C₀₋₂ alkylene)-O-cycloalkyl, SO₂—(C₀₋₂ alkylene)-cycloalkyl, —(C₀₋₂ alkylene)SO₂-cycloalkyl, —CONH-cycloalkyl, —NHCO-cycloalkyl, —NH-cycloalkyl, —O-cycloalkyl, —CO-cycloalkyl, SO₂-cycloalkyl, —(C₀₋₂ alkylene)-heterocycloalkyl, —CO—(C₀₋₂ alkylene)-heterocycloalkyl, —(C₀₋₂ alkylene)-CO-heterocycloalkyl, —CONH—(C₀₋₂ alkylene)-heterocycloalkyl, —(C₀₋₂ alkylene)-CONH-heterocycloalkyl, —NHCO—(C₀₋₂ alkylene)-heterocycloalkyl, —(C₀₋₂ alkylene)-NHCO-heterocycloalkyl, —NH—(C₀₋₂ alkylene)-heterocycloalkyl, —(C₀₋₂ alkylene)-NH-heterocycloalkyl, —O—(C₀₋₂ alkylene)-heterocycloalkyl, —(C₀₋₂ alkylene)-O-heterocycloalkyl, SO₂—(C₀₋₂ alkylene)-heterocycloalkyl, —(C₀₋₂ alkylene)SO₂-heterocycloalkyl, —CONH-heterocycloalkyl, —NHCO-heterocycloalkyl, —NH— heterocycloalkyl, —O-heterocycloalkyl, —CO-heterocycloalkyl, SO₂-heterocycloalkyl, —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, SO₂—(C₀₋₂ alkylene)-aryl, —(C₀₋₂ alkylene)SO₂-aryl, —CONH-aryl, —NHCO-aryl, —NH-aryl, —O-aryl, —CO-aryl, SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)heteroaryl, —(C₀₋₂ alkylene)-NH-heteroaryl, —O—(C₀₋₂ alkylene)heteroaryl, —(C₀₋₂ alkylene)-O-heteroaryl, SO₂—(C₀₋₂ alkylene)heteroaryl, —(C₀₋₂ alkylene)SO₂-heteroaryl, —CONH-heteroaryl, —NHCO-heteroaryl, —NH-heteroaryl, —O-heteroaryl, —CO-heteroaryl, and SO₂-heteroaryl, wherein said cycloalkyl, heterocycloalkyl, aryl or heteroaryl is optionally substituted with one or more groups independently selected from halogen, CN, OH, C₁₋₅ alkyl, C₁₋₅ haloalkyl, O(C₁₋₅ alkyl), O(C₁₋₅ haloalkyl), SH, S(C₁₋₅ alkyl), S(C₁₋₅ haloalkyl), NH₂, NH(C₁₋₅ alkyl), NH(C₁₋₅ haloalkyl), N(C₁₋₅ alkyl)(C₁₋₅ alkyl), N(C₁₋₅ haloalkyl)(C₁₋₅ alkyl), CONH₂, CONH(C₁₋₅ alkyl), and CON(C₁₋₅ alkyl)(C₁₋₅ alkyl),
   preferably wherein R₄ is selected from —(C₀₋₂ alkylene)aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, —SO₂—(C₀₋₂ alkylene)-aryl, —(C₀₋₂ alkylene)-SO₂-aryl, —CONH-aryl, —NHCO-aryl, —NH-aryl, —O-aryl, —CO-aryl, —SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, —SO₂—(C₀₋₂ alkylene)-heteroaryl, —(C₀₋₂ alkylene)-SO₂-heteroaryl, —CONH-heteroaryl, —NHCO-heteroaryl, —NH-heteroaryl, —O-heteroaryl, —CO-heteroaryl, and —SO₂-heteroaryl, wherein said aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl),
   or wherein R₄ is selected from C₁₋₁₂ alkyl, C₂₋₁₂ alkenyl, C₂₋₁₂ alkynyl, cycloalkyl, heterocycloalkyl, aryl, and heteroaryl, preferably wherein R₄ is selected from cycloalkyl, heterocycloalkyl, aryl, and heteroaryl, more preferably wherein R₄ is selected from aryl, and heteroaryl, even more preferably wherein R₄ is heteroaryl, wherein said alkyl, alkenyl, or alkynyl is optionally substituted with one or more groups independently selected from halogen, —CN, —OH, —O(C₁₋₅ alkyl), —O(C₁₋₅ haloalkyl), SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl),
   preferably wherein R₄ is a five membered heteroaryl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably wherein the five membered heteroaryl is selected from imidazolyl, isoxazolyl, pyrazolyl, 1,2,3-triazolyl, 1,2,4-triazolyl, thiazolyl, 1,2,4-oxadiazolyl, 1,3,4-oxadiazolyl, 1,2,4-thiadiazolyl, or 1,3,4-thiadiazolyl, preferably wherein the five membered heteroaryl is 1,2,4-thiadiazolyl, optionally substituted with one or more groups independently selected from halogen, —CN, —OH, C₁₋₅ alkyl, C₁₋₅ haloalkyl, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CONH₂, —CONH(C₁₋₅ alkyl), and —CON(C₁₋₅ alkyl)(C₁₋₅ alkyl), preferably optionally substituted with C₁₋₅ alkyl, C₁₋₅ haloalkyl, —O(C₁₋₅ alkyl), —SH, —S(C₁₋₅ alkyl), more preferably optionally substituted with C₁₋₅ alkyl, C₁₋₅ haloalkyl, even more preferably optionally substituted with C₁₋₅ haloalkyl, preferably selected from —CH₂F, —CHF₂ and CF₃, most preferably optionally substituted with —CHF₂.
13. A pharmaceutical composition comprising the compound of any one of paragraphs 1 to 12 or a pharmaceutically acceptable salt, hydrate or solvate thereof, and a pharmaceutically acceptable carrier.
14. The compound of any one of paragraphs 1 to 12 or a pharmaceutically acceptable salt, hydrate or solvate thereof, or a pharmaceutical composition of paragraph 13, for use in therapy.
15. The compound for use or the pharmaceutical composition for use of paragraph 14, for use in a method of treating a disease or disorder in which PARG activity is implicated,
   or for use in a method of treating a proliferative disorder, preferably wherein the proliferative disorder is cancer, preferably a human cancer.

1. A compound of formula (I): 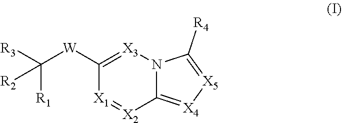
or an enantiomer, diastereoisomer, tautomer, pharmaceutically acceptable solvate, pharmaceutically acceptable crystal form, pharmaceutically acceptable salt or a prodrug thereof, wherein:
R₁ is selected from the group consisting of hydrogen, chloro, fluoro, cyano, formyl, (C₁₋₂)alkyl, (C₂)alkenyl, (C₂)alkynyl, (C₁₋₂)haloalkyl-(C₁₋₂ alkylene)-OH and —(C₁₋₂ alkylene)-O—(C₁₋₂ alkyl);
R₂ and R₃ are independently each (C₁₋₂)alkyl or (C₁₋₂)haloalkyl, or R₂ and R₃ together with the carbon atom to which they are attached form cyclopropyl;
W is selected from —NHS(O)_{y}—, —S(O)_{y}NH—, —NHS(O)(NH)—, —NHS(O)(NCH₃)—, —S(O)(NH)—NH—, —S(O)(NCH₃)—NH—, wherein y is 1 or 2;
X₁ and X₃ are independently selected from the group consisting of N, CH, C(C₁₋₂ alkyl), C—Cl and C—F;
X₂ is N or C—Y_{C2}—R_{C2},
wherein Y_{C2} is selected from a covalent bond, C₁₋₅ alkylene, C₂₋₅ alkenylene, C₂₋₅ alkynylene, cycloalkylene, cycloalkenylene, heterocycloalkylene and heterocycloalkenylene, wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and further wherein said cycloalkylene, said cycloalkenylene, said heterocycloalkylene and said heterocycloalkenylene are each optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₋₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₋₅ alkyl), —(C₁₋₅ alkylene)-S(C₁₋₅ haloalkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), and —(C₁₋₅ alkylene)N(C₁₋₅ alkyl)CON(C₁₋₅ alkyl)(C₁₋₅ alkyl), preferably selected from halogen, CN, OH, C₁₋₅ alkyl, O(C₁₋₅ alkyl), SH, S(C₁₋₅ alkyl), NH₂, NH(C₁₋₅ alkyl), and N(C₁₋₅ alkyl)(C₁₋₅ alkyl), and
wherein R_{C2} is selected from hydrogen, halo, —OH, —NH₂, —SH, —CN, C₁₋₁₂ alkyl, C₂₋₁₂ alkenyl, C₂₋₁₂ alkynyl, cycloalkyl, cycloalkenyl, heterocycloalkyl, heterocycloalkenyl, aryl, and heteroaryl,
wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))((C₁₋₅ alkyl)), and —P(O)(O(C₁₋₅ alkyl))(C₁₋₅ alkyl), preferably selected from halogen, —CN, —OH, —O(C₁₋₅ alkyl)-, —O(C₁₋₅ haloalkyl)-, C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), preferably selected from halogen, —CN, —OH, —O(C₁₋₅ alkyl), —(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), and
wherein said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO— cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —COO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl);
X₄ is N or C—R_{C4},
wherein R_{C4} is selected from hydrogen, halo, C₁₋₆ alkyl, C₂₋₆ alkynyl, —O(C₁₋₆ alkyl), —S(C₁₋₅ alkyl), —NH(C₁₋₅ alkyl), —N(C₁₋₅ alkyl)(C₁₋₆ alkyl), —CO(C₁₋₆ alkyl), C₁₋₆ haloalkyl, —O(C₁₋₅ haloalkyl), —S(C₁₋₆ haloalkyl), —NH(C₁₋₆ haloalkyl), —N(C₁₋₆ haloalkyl)₂, —CO(C₁₋₆ haloalkyl), —(C₀₋₃ alkylene)cycloalkyl, —O—(C₀₋₃ alkylene)-cycloalkyl, —CO—(C₀₋₃ alkylene)-cycloalkyl, —(C₀₋₃ alkylene)cycloalkenyl, —O—(C₀₋₃ alkylene)-cycloalkenyl, —CO—(C₀₋₃ alkylene)-cycloalkenyl, —(C₀₋₃ alkylene)-heterocycloalkyl, —O—(C₀₋₃ alkylene)-heterocycloalkyl, —CO—(C₀₋₃ alkylene)-heterocycloalkyl, —(C₀₋₃ alkylene)-heterocycloalkenyl, —O—(C₀₋₃ alkylene)-heterocycloalkenyl, —CO—(C₀₋₃ alkylene)-heterocycloalkenyl, —(C₀₋₃ alkylene)-aryl, —O—(C₀₋₃ alkylene)-aryl, —CO—(C₀₋₃ alkylene)-aryl, —(C₀₋₃ alkylene)-heteroaryl, —O—(C₀₋₃ alkylene)-heteroaryl and —CO—(C₀₋₃ alkylene)-heteroaryl,
wherein said alkyl or said alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), preferably selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and
wherein said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl);
X₅ is N or C—R_{C5},
wherein R_{C5} is selected from hydrogen, halo, C₁₋₆ alkyl, —O(C₁₋₆ alkyl), —S(C₁₋₆ alkyl), —NH(C₁₋₆ alkyl), —N(C₁₋₆ alkyl)(C₁₋₆ alkyl) and C₁₋₆ haloalkyl;
R₄ is Y_{R5}—R_{R5},
wherein Y_{R5} is selected from a covalent bond, C₁₋₄ alkylene, C₂₋₄ alkenylene, and C₂₋₄ alkynylene, wherein said alkylene, said alkenylene and said alkynylene are each optionally substituted with one or more groups independently selected from halogen, —CN, —OH, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), and —N(C₁₋₅ haloalkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, —COO—, S—, —SO—, and SO₂—, and
wherein R_{R5} is selected from C₁₋₁₂ alkyl, C₁₋₁₂ alkenyl, C₂₋₁₂ alkynyl, cycloalkyl, cycloalkenyl, heterocycloalkyl, heterocycloalkenyl, aryl, and heteroaryl,
wherein said alkyl, alkenyl, or alkynyl is optionally substituted with one or more groups independently selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, cycloalkenyl, heterocycloalkyl, heterocycloalkenyl, aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

2. The compound of claim 1, wherein R₁ is selected from the group consisting of hydrogen, chloro, fluoro, cyano, formyl, (C₁₋₂)alkyl, (C₂)alkenyl, (C₂)alkynyl and (C₁₋₂)haloalkyl.

3. The compound of claim 1 or 2, wherein R₁ is selected from the group consisting of chloro, fluoro, cyano, formyl, (C₁₋₂)alkyl, (C₂)alkenyl, (C₂)alkynyl and (C₁₋₂)haloalkyl.

4. The compound of any one of claims 1 to 3, wherein R₁ is selected from the group consisting of cyano, (C₁₋₂)alkyl, and (C₁₋₂)haloalkyl.

5. The compound of any one of claims 1 to 4, wherein R₁ is selected from the group consisting of cyano, methyl and fluoromethyl.

6. The compound of any one of claims 1 to 5, wherein R₁ is cyano.

7. The compound of any one of claims 1 to 5, wherein R₁ is methyl.

8. The compound of any one of claims 1 to 5, wherein R₁ is fluoromethyl.

9. The compound of any one of claims 1 to 9, wherein R₂ and R₃ together with the carbon atom to which they are attached form cyclopropyl.

10. The compound of any one of claims 1 to 9, wherein W is —NHS(O)₂—, preferably wherein the left side of W as defined herein is attached to the carbon atom that carries R₁, R₂ and R₃, and the right side of W as defined herein is attached to the ring system shown in formula (I).

11. The compound of any one of claims 1 to 10, wherein X₁ and X₃ are independently selected from the group consisting of N, CH and CF.

12. The compound of any one of claims 1 to 11, wherein X₁ and X₃ are each CH.

13. The compound of any one of claims 1 to 12, wherein Y_{C2} is selected from a covalent bond, C₁₋₅ alkylene, C₂₋₅ alkenylene, C₂₋₅ alkynylene, cycloalkylene and heterocycloalkylene wherein said alkylene, said alkenylene and said alkynylene are each optionally substituted with one or more groups independently selected from halogen, CN, OH, O(C₁₋₅ alkyl), —O(C₁₋₅ haloalkyl), C₁₋₅ haloalkyl, SH, S(C₁₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), preferably selected from halogen, CN, OH, O(C₁₋₅ alkyl), SH, S(C₁₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and further wherein said cycloalkylene and said heterocycloalkylene are each optionally substituted with one or more groups independently selected from halogen, CN, OH, C₁₋₅ alkyl, C₁₋₅ haloalkyl, O(C₁₋₅ alkyl), —O(C₁₋₅ haloalkyl), SH, S(C₁₅ alkyl), —S(C₁₋₅ haloalkyl), NH₂, NH(C₁₋₅ alkyl), —NH(C₁₋₅ haloalkyl), N(C₁₋₅ alkyl)(C₁₋₅ alkyl), N(C₁₋₅ haloalkyl)(C₁₋₅ alkyl), —(N-heterocycloalkyl), —CO(C₁₋₅ alkyl), CONH₂, CONH(C₁₋₅ alkyl), CON(C₁₋₅ alkyl)(C₁₋₅ alkyl), —CO—(N-heterocycloalkyl), NHCO—(C₁₋₅ alkyl), N(C₁₋₅ alkyl)-CO—(C₁₋₅ alkyl), NHCONH₂, NHCONH—(C₁₋₅ alkyl), NHCON(C₁₋₅ alkyl)(C₁₋₅ alkyl), N(C₁₋₅ alkyl)CONH₂, N(C₁₋₅ alkyl)CONH—(C₁₋₅ alkyl), and N(C₁₋₅ alkyl)CON(C₁₋₅ alkyl)(C₁₋₅ alkyl), —(C₁₋₅ alkylene)-CN, —(C₁₋₅ alkylene)OH, —(C₁₋₅ alkylene)O(C₁₋₅ alkyl), —(C₁₋₅ alkylene)-O(C₁₋₅ haloalkyl), —(C₁₋₅ alkylene)SH, —(C₁₋₅ alkylene)S(C₁₅ alkyl), —(C₁₋₅ alkylene)-S(C₁₋₅ haloalkyl), —(C₁₋₅ alkylene)NH₂, —(C₁₋₅ alkylene)NH(C₁₋₅ alkyl), —(C₁₋₅ alkylene)-NH(C₁₋₅ haloalkyl), —(C₁₋₅ alkylene)N(C₁₋₅ alkyl)(C₁₋₅ alkyl), —(C₁₋₅ alkylene)N(C₁₋₅ alkyl)(C₁₋₅ haloalkyl), —(C₁₋₅ alkylene)(N-heterocycloalkyl), —(C₁₋₅ alkylene)N(C₁₋₅ haloalkyl)(C₁₋₅ alkyl), —(C₁₋₅ alkylene)-CO(C₁₋₅ alkyl), —(C₁₋₅ alkylene)CONH₂, —(C₁₋₅ alkylene)CONH(C₁₋₅ alkyl), —(C₁₋₅ alkylene)CON(C₁₋₅ alkyl)(C₁₋₅ alkyl), —(C₁₋₅ alkylene)CO—(N-heterocycloalkyl), —(C₁₋₅ alkylene)NHCO—(C₁₋₅ alkyl), —(C₁₋₅ alkylene)N(C₁₋₅ alkyl)-CO—(C₁₋₅ alkyl), —(C₁₋₅ alkylene)NHCONH₂, —(C₁₋₅ alkylene)NHCONH—(C₁₋₅ alkyl), —(C₁₋₅ alkylene)NHCON(C₁₋₅ alkyl)(C₁₋₅ alkyl), —(C₁₋₅ alkylene)N(C₁₋₅ alkyl)CONH₂, —(C₁₋₅ alkylene)N(C₁₋₅ alkyl)CONH—(C₁₋₅ alkyl), and —(C₁₋₅ alkylene)N(C₁₋₅ alkyl)CON(C₁₋₅ alkyl)(C₁₋₅ alkyl), preferably selected from halogen, CN, OH, C₁₋₅ alkyl, O(C₁₋₅ alkyl), SH, S(C₁₅ alkyl), NH₂, NH(C₁₋₅ alkyl), and N(C₁₋₅ alkyl)(C₁₋₅ alkyl).

14. The compound of any one of claims 1 to 13, wherein X₂ is C—Y_{C2}—R_{C2}, wherein —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂₋₁₂ alkenyl, —O—C₂₋₁₂ alkynyl, —NH—C₂₋₁₂ alkynyl, —N(C₁₋₅ alkyl)-C₂₋₁₂ alkynyl, (C₀₋₃ alkylene)-cycloalkyl, —CO—(C₀₋₃ alkylene)cycloalkyl, (C₀₋₃ alkylene)-CO-cycloalkyl, —CONH—(C₀₋₃ alkylene)cycloalkyl, —(C₀₋₃ alkylene)-CONH-cycloalkyl, —NHCO—(C₀₋₃ alkylene)cycloalkyl, (C₀₋₃ alkylene)-NHCO-cycloalkyl, —NH—(C₀₋₃ alkylene)cycloalkyl, —(C₀₋₃ alkylene)-NH-cycloalkyl, —O—(C₀₋₃ alkylene)cycloalkyl, —(C₀₋₃ alkylene)-O-cycloalkyl, —SO₂—(C₀₋₃ alkylene)cycloalkyl, —(C₀₋₃ alkylene)-SO₂-cycloalkyl, —CONH-cycloalkyl, —NHCO-cycloalkyl, —NH-cycloalkyl, —O-cycloalkyl, —CO-cycloalkyl, —SO₂-cycloalkyl, (C₀₋₃ alkylene)-cycloalkenyl, —CO—(C₀₋₃ alkylene)cycloalkenyl, (C₀₋₃ alkylene)-CO-cycloalkenyl, —CONH—(C₀₋₃ alkylene)cycloalkenyl, —(C₀₋₃ alkylene)-CONH-cycloalkenyl, —NHCO—(C₀₋₃ alkylene)cycloalkenyl, (C₀₋₃ alkylene)-NHCO-cycloalkenyl, —NH—(C₀₋₃ alkylene)cycloalkenyl, —(C₀₋₃ alkylene)-NH-cycloalkenyl, —O—(C₀₋₃ alkylene)cycloalkenyl, —(C₀₋₃ alkylene)-O-cycloalkenyl, —SO₂—(C₀₋₃ alkylene)cycloalkenyl, —(C₀₋₃ alkylene)-SO₂-cycloalkenyl, —CONH-cycloalkenyl, —NHCO-cycloalkenyl, —NH-cycloalkenyl, —O-cycloalkenyl, —CO— cycloalkenyl, —SO₂-cycloalkenyl, —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, —(C₀₋₃ alkylene)-O-heterocycloalkyl, —SO₂—(C₀₋₃ alkylene)heterocycloalkyl, —(C₀₋₃ alkylene)-SO₂-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)-heterocycloalkenyl, —CO—(C₀₋₃ alkylene)heterocycloalkenyl, —(C₀₋₃ alkylene)-CO-heterocycloalkenyl, —CONH—(C₀₋₃ alkylene)heterocycloalkenyl, —(C₀₋₃ alkylene)-CONH-heterocycloalkenyl, —NHCO—(C₀₋₃ alkylene)heterocycloalkenyl, —(C₀₋₃ alkylene)-NHCO-heterocycloalkenyl, —NH—(C₀₋₃ alkylene)heterocycloalkenyl, —(C₀₋₃ alkylene)-NH-heterocycloalkenyl, —O—(C₀₋₃ alkylene) heterocycloalkenyl, —(C₀₋₃ alkylene)-O-heterocycloalkenyl, —SO₂—(C₀₋₃ alkylene)heterocycloalkenyl, —(C₀₋₃ alkylene)-SO₂-heterocycloalkenyl, —CONH-heterocycloalkenyl, —NHCO-heterocycloalkenyl, —NH-heterocycloalkenyl, —O-heterocycloalkenyl, —CO-heterocycloalkenyl, —SO₂-heterocycloalkenyl, (C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, (C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH— heteroaryl, —O-heteroaryl, —CO-heteroaryl and —SO₂-heteroaryl, preferably —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂₋₁₂ alkenyl, —O—C₂₋₁₂ alkynyl, —NH—C₂₋₁₂ alkynyl, —N(C₁₋₅ alkyl)-C₂₋₁₂ alkynyl, (C₀₋₃ alkylene)-cycloalkyl, —CO—(C₀₋₃ alkylene)cycloalkyl, (C₀₋₃ alkylene)-CO-cycloalkyl, —CONH—(C₀₋₃ alkylene)cycloalkyl, —(C₀₋₃ alkylene)-CONH-cycloalkyl, —NHCO—(C₀₋₃ alkylene)cycloalkyl, (C₀₋₃ alkylene)-NHCO-cycloalkyl, —NH—(C₀₋₃ alkylene)cycloalkyl, —(C₀₋₃ alkylene)-NH-cycloalkyl, —O—(C₀₋₃ alkylene)cycloalkyl, —(C₀₋₃ alkylene)-O-cycloalkyl, —SO₂—(C₀₋₃ alkylene)cycloalkyl, —(C₀₋₃ alkylene)-SO₂-cycloalkyl, —CONH-cycloalkyl, —NHCO-cycloalkyl, —NH-cycloalkyl, —O-cycloalkyl, —CO-cycloalkyl, —SO₂-cycloalkyl, —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, —(C₀₋₃ alkylene)-O-cycloalkyl, —SO₂—(C₀₋₃ alkylene)heterocycloalkyl, —(C₀₋₃ alkylene)-SO₂-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, (C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, (C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —O(C₁₋₅ alkyl), —O(C₁₋₅ haloalkyl), —C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl) and wherein said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl)

15. The compound of any one of claims 1 to 14, wherein —Y_{C2}—R_{C2} is selected from —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, (C₀₋₃ alkylene)-O-cycloalkyl, (C₀₋₃ alkylene)-O-heterocycloalkyl, —SO₂—(C₀₋₃ alkylene)heterocycloalkyl, —(C₀₋₃ alkylene)-SO₂-heterocycloalkyl, —CONH-heterocycloalkyl, —NHCO— heterocycloalkyl, —NH-heterocycloalkyl, —O-heterocycloalkyl, —CO-heterocycloalkyl, —SO₂-heterocycloalkyl, —(C₀₋₃ alkylene)-heterocycloalkenyl, —CO—(C₀₋₃ alkylene)heterocycloalkenyl, —(C₀₋₃ alkylene)-CO-heterocycloalkenyl, —CONH—(C₀₋₃ alkylene)heterocycloalkenyl, —(C₀₋₃ alkylene)-CONH-heterocycloalkenyl, —NHCO—(C₀₋₃ alkylene)heterocycloalkenyl, —(C₀₋₃ alkylene)-NHCO-heterocycloalkenyl, —NH—(C₀₋₃ alkylene)heterocycloalkenyl, —(C₀₋₃ alkylene)-NH-heterocycloalkenyl, —O—(C₀₋₃ alkylene) heterocycloalkenyl, (C₀₋₃ alkylene)-O-heterocycloalkenyl, —SO₂—(C₀₋₃ alkylene)heterocycloalkenyl, —(C₀₋₃ alkylene)-SO₂-heterocycloalkenyl, —CONH-heterocycloalkenyl, —NHCO-heterocycloalkenyl, —NH-heterocycloalkenyl, —O-heterocycloalkenyl, —CO— heterocycloalkenyl, —SO₂-heterocycloalkenyl, —(C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, —(C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, preferably —Y_{C2}—R_{C2} is selected from —(C₀₋₃ alkylene)-heterocycloalkyl, —CO—(C₀₋₃ alkylene)heterocycloalkyl, —(C₀₋₃ alkylene)-CO-heterocycloalkyl, —CONH—(C₀₋₃ alkylene)heterocycloalkyl, —(C₀₋₃ alkylene)-CONH-heterocycloalkyl, —NHCO—(C₀₋₃ alkylene)heterocycloalkyl, —(C₀₋₃ alkylene)-NHCO-heterocycloalkyl, —NH—(C₀₋₃ alkylene)heterocycloalkyl, —(C₀₋₃ alkylene)-NH-heterocycloalkyl, —O—(C₀₋₃ alkylene) heterocycloalkyl, (C₀₋₃ alkylene)-O-cycloalkyl, —SO₂—(C₀₋₃ alkylene)heterocycloalkyl, —(C₀₋₃ alkylene)-SO₂-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)aryl, —CO—(C₀₋₃ alkylene)aryl, —(C₀₋₃ alkylene)-CO-aryl, —CONH—(C₀₋₃ alkylene)aryl, —(C₀₋₃ alkylene)-CONH-aryl, —NHCO—(C₀₋₃ alkylene)aryl, —(C₀₋₃ alkylene)-NHCO-aryl, —NH—(C₀₋₃ alkylene)aryl, —(C₀₋₃ alkylene)-NH-aryl, —O—(C₀₋₃ alkylene)aryl, —(C₀₋₃ alkylene)-O-aryl, —SO₂—(C₀₋₃ alkylene)aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)heteroaryl, —CO—(C₀₋₃ alkylene)heteroaryl, —(C₀₋₃ alkylene)-CO-heteroaryl, —CONH—(C₀₋₃ alkylene)heteroaryl, —(C₀₋₃ alkylene)-CONH-heteroaryl, —NHCO—(C₀₋₃ alkylene)heteroaryl, —(C₀₋₃ alkylene)-NHCO-heteroaryl, —NH—(C₀₋₃ alkylene)heteroaryl, —(C₀₋₃ alkylene)-NH-heteroaryl, —O—(C₀₋₃ alkylene)heteroaryl, —(C₀₋₃ alkylene)-O-heteroaryl, —SO₂—(C₀₋₃ alkylene)heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-C(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl).

16. The compound of any one of claims 1 to 15, wherein —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)-heterocycloalkenyl, —CONH-heterocycloalkenyl, —NHCO-heterocycloalkenyl, —NH-heterocycloalkenyl, —O— heterocycloalkenyl, —CO-heterocycloalkenyl, —SO₂-heterocycloalkenyl, —(C₀₋₃ alkylene)aryl, —CONH-aryl, —NHCO-aryl, —NH-aryl, —O-aryl, —CO-aryl, —SO₂-aryl, —(C₀₋₃ alkylene)heteroaryl, —CONH-heteroaryl, —NHCO-heteroaryl, —NH-heteroaryl, —O-heteroaryl, —CO-heteroaryl and —SO₂-heteroaryl, preferably —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O-heterocycloalkyl, —CO-heterocycloalkyl, —SO₂-heterocycloalkyl, —(C₀₋₃ alkylene)aryl, —CONH-aryl, —NHCO-aryl, —NH-aryl, —O-aryl, —CO-aryl, —SO₂-aryl, —(C₀₋₃ alkylene)heteroaryl, —CONH-heteroaryl, —NHCO-heteroaryl, —NH-heteroaryl, —O— heteroaryl, —CO-heteroaryl and —SO₂-heteroaryl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl).

17. The compound of any one of claims 1 to 16, wherein —Y_{C2}—R_{C2} is selected from —(C₀₋₃ alkylene)-heterocycloalkyl, —(C₀₋₃ alkylene)-heterocycloalkenyl, —(C₀₋₃ alkylene)aryl, and —(C₀₋₃ alkylene)heteroaryl, preferably —Y_{C2}—R_{C2} is selected from —(C₀₋₃ alkylene)-heterocycloalkyl, —(C₀₋₃ alkylene)aryl, and —(C₀₋₃ alkylene)heteroaryl, wherein said heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CO(C₁₋₅ haloalkyl), —CO-cycloalkyl, —COO(C₁₋₅ alkyl), —COO(C₁₋₅ haloalkyl), —COO-cycloalkyl, —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —OCONH₂, —OCONH—(C₁₋₅ alkyl), —OCON(C₁₋₅ alkyl)(C₁₋₅ alkyl), —NHCOO(C₁₋₅ alkyl), —N(C₁₋₅ alkyl)COO—(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CO(C₁₋₅ haloalkyl), —(C₁₋₅ alkylene)-CO-cycloalkyl, —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), —(C₁₋₅ alkylene)-N(C₁₋₅ alkyl)CON(C₁₋₅ alkyl)(C₁₋₅ alkyl), —(C₁₋₅ alkylene)-OCONH₂, —(C₁₋₅ alkylene)-OCONH—(C₁₋₅ alkyl), —(C₁₋₅ alkylene)-OCON(C₁₋₅ alkyl)(C₁₋₅ alkyl), —(C₁₋₅ alkylene)-NHCOO(C₁₋₅ alkyl), and —(C₁₋₅ alkylene)-N(C₁₋₅ alkyl)COO—(C₁₋₅ alkyl).

18. The compound of any one of claims 1 to 12,
wherein Y_{C2} is selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, CN, OH, O(C₁₋₅ alkyl), SH, S(C₁₋₅ alkyl), NH₂, NH(C₁₋₅ alkyl), and N(C₁₋₅ alkyl)(C₁₋₅ alkyl), and further wherein one or more —CH₂— units comprised in said alkylene, said alkenylene or said alkynylene are each optionally replaced by a group independently selected from —O—, NH—, N(C₁₋₅ alkyl)-, CO—, S—, —SO—, and SO₂—, and
wherein R_{C2} is selected from hydrogen, halo, —OH, —NH₂, —SH, —CN, C₁₋₁₂ alkyl, C₂₋₁₂ alkenyl, C₂₋₁₂ alkynyl, cycloalkyl, heterocycloalkyl, aryl, and heteroaryl,
wherein said alkyl, alkenyl, or alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), and —P(O)(O(C₁₋₅ alkyl))(C₁₋₅ alkyl), preferably selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), and
wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(O)(C₁₋₅ alkyl), —S(O)₂(C₁₋₅ alkyl), —S(O)(NH)(C₁₋₅ alkyl), —S(O)(N(C₁₋₅ alkyl))(C₁₋₅ alkyl), —N═S(O)(C₁₋₅ alkyl)(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), —N(C₁₋₅ alkyl)CON(C₁₋₅ alkyl)(C₁₋₅ alkyl), —P(O)(C₁₋₅ alkyl)(C₁₋₅ alkyl), —P(O)(O(C₁₋₅ alkyl))(O(C₁₋₅ alkyl)), —P(O)(O(C₁₋₅ alkyl))(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-S(O)(C₁₋₅ alkyl), —(C₁₋₅ alkylene)-S(O)₂(C₁₋₅ alkyl), —(C₁₋₅ alkylene)-S(O)(NH)(C₁₋₅ alkyl), —(C₁₋₅ alkylene)-S(O)(N(C₁₋₅ alkyl))(C₁₋₅ alkyl), —(C₁₋₅ alkylene)-P(O)(C₁₋₅ alkyl)(C₁₋₅ alkyl), —(C₁₋₅ alkylene)-P(O)(O(C₁₋₅ alkyl))(O(C₁₋₅ alkyl)), —(C₁₋₅ alkylene)-P(O)(O(C₁₋₅ alkyl))(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —(C₁₋₅ alkylene)-OH, —(C₁₋₅ alkylene)-O(C₁₋₅ alkyl), —(C₁₋₅ alkylene)-O(C₁₋₅ haloalkyl), —SH, —S(C₁₋₅ alkyl), —S(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), more preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

19. The compound of claim 18, wherein X₂ is C—Y_{C2}—R_{C2}, wherein —Y_{C2}—R_{C2} is selected from —O—C₁₋₁₂ alkyl, —NH—C₁₋₁₂ alkyl, —N(C₁₋₅ alkyl)-C₁₋₁₂ alkyl, —O—C₂₋₁₂ alkenyl, —NH—C₂₋₁₂ alkenyl, —N(C₁₋₅ alkyl)-C₂-12 alkenyl, —O—C₂₋₁₂ alkynyl, —NH—C₂₋₁₂ alkynyl, —N(C₁₋₅ alkyl)-C₂₋₁₂ alkynyl, —(C₀₋₃ alkylene)-cycloalkyl, —CO—(C₀₋₃ alkylene)-cycloalkyl, —(C₀₋₃ alkylene)-CO-cycloalkyl, —CONH—(C₀₋₃ alkylene)-cycloalkyl, (C₀₋₃ alkylene)-CONH-cycloalkyl, —NHCO—(C₀₋₃ alkylene)-cycloalkyl, —(C₀₋₃ alkylene)-NHCO-cycloalkyl, —NH—(C₀₋₃ alkylene)-cycloalkyl, —(C₀₋₃ alkylene)-NH-cycloalkyl, —O—(C₀₋₃ alkylene)-cycloalkyl, —(C₀₋₃ alkylene)-O-cycloalkyl, —SO₂—(C₀₋₃ alkylene)-cycloalkyl, —(C₀₋₃ alkylene)-SO₂-cycloalkyl, —CONH-cycloalkyl, —NHCO-cycloalkyl, —NH-cycloalkyl, —O-cycloalkyl, —CO— cycloalkyl, —SO₂-cycloalkyl, —(C₀₋₃ alkylene)-heterocycloalkyl, —CO—(C₀₋₃ alkylene)-heterocycloalkyl, —(C₀₋₃ alkylene)-CO-heterocycloalkyl, —CONH—(C₀₋₃ alkylene)-heterocycloalkyl, —(C₀₋₃ alkylene)-CONH-heterocycloalkyl, —NHCO—(C₀₋₃ alkylene)-heterocycloalkyl, —(C₀₋₃ alkylene)-NHCO-heterocycloalkyl, —NH—(C₀₋₃ alkylene)-heterocycloalkyl, —(C₀₋₃ alkylene)-NH-heterocycloalkyl, —O—(C₀₋₃ alkylene)-heterocycloalkyl, —(C₀₋₃ alkylene)-O-cycloalkyl, —SO₂—(C₀₋₃ alkylene)-heterocycloalkyl, —(C₀₋₃ alkylene)-SO₂-heterocycloalkyl, —CONH-heterocycloalkyl, —NHCO-heterocycloalkyl, —NH-heterocycloalkyl, —O-heterocycloalkyl, —CO-heterocycloalkyl, —SO₂-heterocycloalkyl, —(C₀₋₃ alkylene)-aryl, —CO—(C₀₋₃ alkylene)-aryl, —(C₀₋₃ alkylene)-CO-aryl, —CONH—(C₀₋₃ alkylene)-aryl, —(C₀₋₃ alkylene)-CONH-aryl, —NHCO—(C₀₋₃ alkylene)-aryl, —(C₀₋₃ alkylene)-NHCO-aryl, —NH—(C₀₋₃ alkylene)-aryl, —(C₀₋₃ alkylene)-NH-aryl, —O—(C₀₋₃ alkylene)-aryl, —(C₀₋₃ alkylene)-O-aryl, —SO₂—(C₀₋₃ alkylene)-aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)-heteroaryl, —CO—(C₀₋₃ alkylene)-heteroaryl, —(C₀₋₃ alkylene)-CO-heteroaryl, —CONH—(C₀₋₃ alkylene)-heteroaryl, —(C₀₋₃ alkylene)-CONH-heteroaryl, —NHCO—(C₀₋₃ alkylene)-heteroaryl, —(C₀₋₃ alkylene)-NHCO-heteroaryl, —NH—(C₀₋₃ alkylene)-heteroaryl, —(C₀₋₃ alkylene)-NH-heteroaryl, —O—(C₀₋₃ alkylene)-heteroaryl, —(C₀₋₃ alkylene)-O-heteroaryl, —SO₂—(C₀₋₃ alkylene)-heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said alkyl, alkenyl, or alkynyl is optionally substituted with one or more groups independently selected from halogen, —CN, —OH, —O(C₁₋₅ alkyl), —O(C₁₋₅ haloalkyl), —C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl) and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

20. The compound of claim 18 or 19, wherein —Y_{C2}—R_{C2} is selected from —(C₀₋₃ alkylene)-heterocycloalkyl, —CO—(C₀₋₃ alkylene)-heterocycloalkyl, —(C₀₋₃ alkylene)-CO-heterocycloalkyl, —CONH—(C₀₋₃ alkylene)-heterocycloalkyl, —(C₀₋₃ alkylene)-CONH-heterocycloalkyl, —NHCO—(C₀₋₃ alkylene)-heterocycloalkyl, —(C₀₋₃ alkylene)-NHCO-heterocycloalkyl, —NH—(C₀₋₃ alkylene)-heterocycloalkyl, —(C₀₋₃ alkylene)-NH-heterocycloalkyl, —O—(C₀₋₃ alkylene)-heterocycloalkyl, —(C₀₋₃ alkylene)-O-cycloalkyl, —SO₂—(C₀₋₃ alkylene)-heterocycloalkyl, —(C₀₋₃ alkylene)-SO₂-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)-aryl, —CO—(C₀₋₃ alkylene)-aryl, —(C₀₋₃ alkylene)-CO-aryl, —CONH—(C₀₋₃ alkylene)-aryl, —(C₀₋₃ alkylene)-CONH-aryl, —NHCO—(C₀₋₃ alkylene)-aryl, —(C₀₋₃ alkylene)-NHCO-aryl, —NH—(C₀₋₃ alkylene)-aryl, —(C₀₋₃ alkylene)-NH-aryl, —O—(C₀₋₃ alkylene)-aryl, —(C₀₋₃ alkylene)-O-aryl, —SO₂—(C₀₋₃ alkylene)-aryl, —(C₀₋₃ alkylene)-SO₂-aryl, —CONH-aryl, —NHCO-aryl, —NH-aryl, —O-aryl, —CO-aryl, —SO₂-aryl, —(C₀₋₃ alkylene)-heteroaryl, —CO—(C₀₋₃ alkylene)-heteroaryl, —(C₀₋₃ alkylene)-CO-heteroaryl, —CONH—(C₀₋₃ alkylene)-heteroaryl, —(C₀₋₃ alkylene)-CONH-heteroaryl, —NHCO—(C₀₋₃ alkylene)-heteroaryl, —(C₀₋₃ alkylene)-NHCO-heteroaryl, —NH—(C₀₋₃ alkylene)-heteroaryl, —(C₀₋₃ alkylene)-NH-heteroaryl, —O—(C₀₋₃ alkylene)-heteroaryl, —(C₀₋₃ alkylene)-O-heteroaryl, —SO₂—(C₀₋₃ alkylene)-heteroaryl, —(C₀₋₃ alkylene)-SO₂-heteroaryl, —CONH-heteroaryl, —NHCO-heteroaryl, —NH-heteroaryl, —O-heteroaryl, —CO-heteroaryl and —SO₂-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

21. The compound of any one of claims 18 to 20, wherein —Y_{C2}—R_{C2} is selected from —(C₀₋₃ alkylene)-heterocycloalkyl, —CONH-heterocycloalkyl, —NHCO-heterocycloalkyl, —NH-heterocycloalkyl, —O— heterocycloalkyl, —CO-heterocycloalkyl, —SO₂-heterocycloalkyl, —(C₀₋₃ alkylene)aryl, —CONH-aryl, —NHCO-aryl, —NH-aryl, —O-aryl, —CO-aryl, —SO₂-aryl, —(C₀₋₃ alkylene)-heteroaryl, —CONH-heteroaryl, —NHCO-heteroaryl, —NH-heteroaryl, —O-heteroaryl, —CO-heteroaryl and —SO₂-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —NH₂, —NH(C₁₋₅ alkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

22. The compound of any one of claims 18 to 21, wherein —Y_{C2}—R_{C2} is selected from —(C₀₋₃ alkylene)-heterocycloalkyl, —(C₀₋₃ alkylene)-aryl, and —(C₀₋₃ alkylene)-heteroaryl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

23. The compound of any one of claims 18 to 22, wherein —Y_{C2}—R_{C2} is selected from heterocycloalkyl, aryl, and heteroaryl, preferably heterocycloalkyl and heteroaryl, more preferably heterocycloalkyl, wherein said heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ haloalkyl), —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

24. The compound of any one of claims 18 to 23, wherein —Y_{C2}—R_{C2} is optionally substituted aryl, preferably —Y_{C2}—R_{C2} is phenyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl),
or wherein —Y_{C2}—R_{C2} is an optionally substituted heteroaryl, preferably wherein —Y_{C2}—R_{C2} is imidazolyl, pyridazinyl, thiazolyl, pyridinyl, pyrimidinyl, pyrazinyl, or indazolyl, wherein heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl),
or wherein —Y_{C2}—R_{C2} is optionally substituted heterocycloalkyl, preferably wherein —Y_{C2}—R_{C2} is morpholinyl, 1,1-dioxothiomorpholinyl, azetinyl, pyrrolidinyl, piperidinyl, 6-oxo-1,6-dihydropyridinyl, or piperazinyl, wherein heterocycloalkyl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl).

25. The compound of any one of claims 18 to 24, wherein —Y_{C2}—R_{C2} is piperazinyl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CO(C₁₋₅ alkyl), —CONH₂, —CONH(C₁₋₅ alkyl), and —CON(C₁₋₅ alkyl)(C₁₋₅ alkyl), preferably wherein —Y_{C2}—R_{C2} is piperazinyl (preferably N-piperazinyl) optionally substituted (preferably N-substituted) with CO(C₁₋₅ alkyl), —CONH₂, —CONH(C₁₋₅ alkyl), and —CON(C₁₋₅ alkyl)(C₁₋₅ alkyl), more preferably wherein —Y_{C2}—R_{C2} is piperazinyl (preferably N-piperazinyl) substituted (preferably N-substituted, preferably at a different N-atom than that attached to the ring system as shown in formula (I)), with —CON(C₁₋₅ alkyl)(C₁₋₅ alkyl), preferably with —CON(CH₃)₂.

26. The compound of any one of claims 1 to 25, wherein X₄ is C—R_{C4}, wherein R_{C4} is selected from hydrogen, halo, C₁₋₆ alkyl, C₂₋₆ alkynyl, —O(C₁₋₆ alkyl), —S(C₁₋₆ alkyl), —NH(C₁₋₆ alkyl), —N(C₁₋₆ alkyl)(C₁₋₆ alkyl), —CO(C₁₋₆ alkyl), C₁₋₆ haloalkyl, —O(C₁₋₆ haloalkyl), —S(C₁₋₆ haloalkyl), —NH(C₁₋₆ haloalkyl), —N(C₁₋₆ haloalkyl)₂, —CO(C₁₋₆ haloalkyl), —(C₀₋₃ alkylene)cycloalkyl, —O—(C₀₋₃ alkylene)-cycloalkyl, —CO—(C₀₋₃ alkylene)-cycloalkyl, —(C₀₋₃ alkylene)-heterocycloalkyl, —O—(C₀₋₃ alkylene)-heterocycloalkyl, —CO—(C₀₋₃ alkylene)-heterocycloalkyl, —(C₀₋₃ alkylene)-aryl, —O—(C₀₋₃ alkylene)-aryl, —CO—(C₀₋₃ alkylene)-aryl, —(C₀₋₃ alkylene)-heteroaryl, —O—(C₀₋₃ alkylene)-heteroaryl and —CO—(C₀₋₃ alkylene)-heteroaryl;
wherein said alkyl or said alkynyl is optionally substituted with one or more groups independently selected from halogen, —CN, —OH, —O(C₁₋₅ alkyl), —O(C₁₋₅ haloalkyl), C₁₋₅ haloalkyl, —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), preferably selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and
wherein said cycloalkyl, cycloalkenyl, heterocycloalkyl, heterocycloalkenyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —(N-heterocycloalkyl), —CO(C₁₋₅ alkyl), —CONH₂, —CONH(C₁₋₅ alkyl), —CON(C₁₋₅ alkyl)(C₁₋₅ alkyl), —CO—(N-heterocycloalkyl), —NHCO—(C₁₋₅ alkyl), —N(C₁₋₅ alkyl)-CO—(C₁₋₅ alkyl), —NHCONH₂, —NHCONH—(C₁₋₅ alkyl), —NHCON(C₁₋₅ alkyl)(C₁₋₅ alkyl), —N(C₁₋₅ alkyl)CONH₂, —N(C₁₋₅ alkyl)CONH—(C₁₋₅ alkyl), and —N(C₁₋₅ alkyl)CON(C₁₋₅ alkyl)(C₁₋₅ alkyl), —(C₁₋₅ alkylene)-CN, —(C₁₋₅ alkylene)-OH, —(C₁₋₅ alkylene)-O(C₁₋₅ alkyl), —(C₁₋₅ alkylene)-O(C₁₋₅ haloalkyl), —(C₁₋₅ alkylene)-SH, —(C₁₋₅ alkylene)-S(C₁₋₅ alkyl), —(C₁₋₅ alkylene)-S(C₁₋₅ haloalkyl), —(C₁₋₅ alkylene)-NH₂, —(C₁₋₅ alkylene)-NH(C₁₋₅ alkyl), —(C₁₋₅ alkylene)-NH(C₁₋₅ haloalkyl), —(C₁₋₅ alkylene)-N(C₁₋₅ alkyl)(C₁₋₅ alkyl), —(C₁₋₅ alkylene)-N(C₁₋₅ alkyl)(C₁₋₅ haloalkyl), —(C₁₋₅ alkylene)-(N-heterocycloalkyl), —(C₁₋₅ alkylene)-N(C₁₋₅ haloalkyl)(C₁₋₅ alkyl), —(C₁₋₅ alkylene)-CO(C₁₋₅ alkyl), —(C₁₋₅ alkylene)-CONH₂, —(C₁₋₅ alkylene)-CONH(C₁₋₅ alkyl), —(C₁₋₅ alkylene)-CON(C₁₋₅ alkyl)(C₁₋₅ alkyl), —(C₁₋₅ alkylene)-CO—(N-heterocycloalkyl), —(C₁₋₅ alkylene)-NHCO—(C₁₋₅ alkyl), —(C₁₋₅ alkylene)-N(C₁₋₅ alkyl)-CO—(C₁₋₅ alkyl), —(C₁₋₅ alkylene)-NHCONH₂, —(C₁₋₅ alkylene)-NHCONH—(C₁₋₅ alkyl), —(C₁₋₅ alkylene)-NHCON(C₁₋₅ alkyl)(C₁₋₅ alkyl), —(C₁₋₅ alkylene)-N(C₁₋₅ alkyl)CONH₂, —(C₁₋₅ alkylene)-N(C₁₋₅ alkyl)CONH—(C₁₋₅ alkyl), and —(C₁₋₅ alkylene)-N(C₁₋₅ alkyl)CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

27. The compound of any one of claims 1 to 26, wherein X₄ is C—R_{C4}, wherein R_{C4} is selected from hydrogen, halo, C₁₋₆ alkyl, C₂₋₆ alkynyl, —O—C₁₋₆ alkyl, —S—C₁₋₆ alkyl, —NH—C₁₋₆ alkyl, C₁₋₆ haloalkyl, —(C₀₋₃ alkylene)-cycloalkyl, —(C₀₋₃ alkylene)-heterocycloalkyl, —(C₀₋₃ alkylene)-aryl and —(C₀₋₃ alkylene)-heteroaryl, wherein said alkyl is optionally substituted with one or more groups independently selected from halogen, —CN, —OH, —O(C₁₋₅ alkyl), —O(C₁₋₅ haloalkyl), —SH, —S(C₁₋₅ alkyl), —O(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ haloalkyl), —S(C₁₋₅ alkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

28. The compound of claim 27, wherein R_{C4} is selected from hydrogen, halo, C₁₋₆ alkyl, C₂₋₆ alkynyl, —O—C₁₋₆ alkyl, —S—C₁₋₅ alkyl, —NH—C₁₋₆ alkyl, and C₁₋₆ haloalkyl, preferably wherein R_{C4} is selected from hydrogen, halo, C₁₋₂ alkyl, and C₂₋₃ alkynyl, more preferably wherein R_{C4} is selected from hydrogen, halo, and C₁₋₂ alkyl, even more preferably wherein R_{C4} is hydrogen or halo.

29. The compound of any one of claims 1 to 28, wherein X₅ is C—R_{C5}, wherein R_{C5} is selected from hydrogen, halo, C₁₋₃ alkyl, —O—C₁₋₃ alkyl, —S—C₁₋₃ alkyl, —NH—C₁₋₃ alkyl, and C₁₋₃ haloalkyl, preferably, wherein R_{C5} is selected from hydrogen, halo, C₁₋₃ alkyl, and C₁₋₃ haloalkyl.

30. The compound of any one of claims 1 to 29, wherein R_{R5} is selected from C₁₋₁₂ alkyl, C₁₋₁₂ alkenyl, C₂₋₁₂ alkynyl, cycloalkyl, heterocycloalkyl, aryl, and heteroaryl,
wherein said alkyl, alkenyl, or alkynyl is optionally substituted with one or more groups independently selected from halogen, —CN, —OH, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, heterocycloalkyl, aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

31. The compound of any one of claims 1 to 29, wherein R₄ is selected from —(C₀₋₂ alkylene)-cycloalkyl, —CO—(C₀₋₂ alkylene)-cycloalkyl, —(C₀₋₂ alkylene)-CO-cycloalkyl, —CONH—(C₀₋₂ alkylene)-cycloalkyl, —(C₀₋₂ alkylene)-CONH-cycloalkyl, —NHCO—(C₀₋₂ alkylene)-cycloalkyl, —(C₀₋₂ alkylene)-NHCO-cycloalkyl, —NH—(C₀₋₂ alkylene)-cycloalkyl, —(C₀₋₂ alkylene)-NH-cycloalkyl, —O—(C₀₋₂ alkylene)-cycloalkyl, —(C₀₋₂ alkylene)-O-cycloalkyl, SO₂—(C₀₋₂ alkylene)-cycloalkyl, —(C₀₋₂ alkylene)SO₂-cycloalkyl, —CONH-cycloalkyl, —NHCO-cycloalkyl, —NH-cycloalkyl, —O-cycloalkyl, —CO-cycloalkyl, SO₂-cycloalkyl, —(C₀₋₂ alkylene)-cycloalkenyl, —CO—(C₀₋₂ alkylene)-cycloalkenyl, —(C₀₋₂ alkylene)-CO-cycloalkenyl, —CONH—(C₀₋₂ alkylene)-cycloalkenyl, —(C₀₋₂ alkylene)-CONH-cycloalkenyl, —NHCO—(C₀₋₂ alkylene)-cycloalkenyl, —(C₀₋₂ alkylene)-NHCO-cycloalkenyl, —NH—(C₀₋₂ alkylene)-cycloalkenyl, —(C₀₋₂ alkylene)-NH-cycloalkenyl, —O—(C₀₋₂ alkylene)-cycloalkenyl, —(C₀₋₂ alkylene)-O-cycloalkenyl, SO₂—(C₀₋₂ alkylene)-cycloalkenyl, —(C₀₋₂ alkylene)SO₂-cycloalkenyl, —CONH-cycloalkenyl, —NHCO— cycloalkenyl, —NH-cycloalkenyl, —O-cycloalkenyl, —CO-cycloalkenyl, SO₂-cycloalkenyl, —(C₀₋₂ alkylene)-heterocycloalkyl, —CO—(C₀₋₂ alkylene)-heterocycloalkyl, —(C₀₋₂ alkylene)-CO-heterocycloalkyl, —CONH—(C₀₋₂ alkylene)-heterocycloalkyl, —(C₀₋₂ alkylene)-CONH-heterocycloalkyl, —NHCO—(C₀₋₂ alkylene)-heterocycloalkyl, —(C₀₋₂ alkylene)-NHCO-heterocycloalkyl, —NH—(C₀₋₂ alkylene)-heterocycloalkyl, —(C₀₋₂ alkylene)-NH-heterocycloalkyl, —O—(C₀₋₂ alkylene)-heterocycloalkyl, —(C₀₋₂ alkylene)-O-heterocycloalkyl, SO₂—(C₀₋₂ alkylene)-heterocycloalkyl, —(C₀₋₂ alkylene)SO₂-heterocycloalkyl, —CONH-heterocycloalkyl, —NHCO-heterocycloalkyl, —NH— heterocycloalkyl, —O-heterocycloalkyl, —CO-heterocycloalkyl, SO₂-heterocycloalkyl, —(C₀₋₂ alkylene)-heterocycloalkenyl, —CO—(C₀₋₂ alkylene)-heterocycloalkenyl, —(C₀₋₂ alkylene)-CO-heterocycloalkenyl, —CONH—(C₀₋₂ alkylene)-heterocycloalkenyl, —(C₀₋₂ alkylene)-CONH-heterocycloalkenyl, —NHCO—(C₀₋₂ alkylene)-heterocycloalkenyl, —(C₀₋₂ alkylene)-NHCO-heterocycloalkenyl, —NH—(C₀₋₂ alkylene)-heterocycloalkenyl, —(C₀₋₂ alkylene)-NH-heterocycloalkenyl, —O—(C₀₋₂ alkylene)-heterocycloalkenyl, —(C₀₋₂ alkylene)-O-heterocycloalkenyl, SO₂—(C₀₋₂ alkylene)-heterocycloalkenyl, —(C₀₋₂ alkylene)SO₂-heterocycloalkenyl, —CONH-heterocycloalkenyl, —NHCO-heterocycloalkenyl, —NH-heterocycloalkenyl, —O-heterocycloalkenyl, —CO-heterocycloalkenyl, SO₂-heterocycloalkenyl, —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, SO₂—(C₀₋₂ alkylene)-aryl, —(C₀₋₂ alkylene)SO₂-aryl, —CONH-aryl, —NHCO-aryl, —NH-aryl, —O-aryl, —CO-aryl, SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, SO₂—(C₀₋₂ alkylene)-heteroaryl, —(C₀₋₂ alkylene)SO₂-heteroaryl, —CONH-heteroaryl, —NHCO-heteroaryl, —NH-heteroaryl, —O-heteroaryl, —CO-heteroaryl, and SO₂-heteroaryl, wherein said cycloalkyl, cycloalkenyl, heterocycloalkyl, heterocycloalkenyl, aryl or heteroaryl is optionally substituted with one or more groups independently selected from halogen, —CN, —OH, —C₁₋₅ alkyl, —C₁₋₅ haloalkyl, —O(C₁₋₅ alkyl), —O(C₁₋₅ haloalkyl), —SH, —S(C₁₋₅ alkyl), —SO(C₁₋₅ alkyl), —SO₂(C₁₋₅ alkyl), —S(C₁₋₅ haloalkyl), —SO(C₁₋₅ haloalkyl), —SO₂(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

32. The compound of any one of claims 1 to 31, wherein R₄ is selected from —(C₀₋₂ alkylene)-cycloalkyl, —CO—(C₀₋₂ alkylene)-cycloalkyl, —(C₀₋₂ alkylene)-CO-cycloalkyl, —CONH—(C₀₋₂ alkylene)-cycloalkyl, —(C₀₋₂ alkylene)-CONH-cycloalkyl, —NHCO—(C₀₋₂ alkylene)-cycloalkyl, —(C₀₋₂ alkylene)-NHCO-cycloalkyl, —NH—(C₀₋₂ alkylene)-cycloalkyl, —(C₀₋₂ alkylene)-NH-cycloalkyl, —O—(C₀₋₂ alkylene)-cycloalkyl, —(C₀₋₂ alkylene)-O-cycloalkyl, —SO₂—(C₀₋₂ alkylene)-cycloalkyl, —(C₀₋₂ alkylene)-SO₂-cycloalkyl, —CONH-cycloalkyl, —NHCO-cycloalkyl, —NH-cycloalkyl, —O-cycloalkyl, —CO— cycloalkyl, —SO₂-cycloalkyl, —(C₀₋₂ alkylene)-heterocycloalkyl, —CO—(C₀₋₂ alkylene)-heterocycloalkyl, —(C₀₋₂ alkylene)-CO-heterocycloalkyl, —CONH—(C₀₋₂ alkylene)-heterocycloalkyl, —(C₀₋₂ alkylene)-CONH-heterocycloalkyl, —NHCO—(C₀₋₂ alkylene)-heterocycloalkyl, —(C₀₋₂ alkylene)-NHCO-heterocycloalkyl, —NH—(C₀₋₂ alkylene)-heterocycloalkyl, —(C₀₋₂ alkylene)-NH-heterocycloalkyl, —O—(C₀₋₂ alkylene)-heterocycloalkyl, —(C₀₋₂ alkylene)-O-heterocycloalkyl, —SO₂—(C₀₋₂ alkylene)-heterocycloalkyl, —(C₀₋₂ alkylene)-SO₂-heterocycloalkyl, —CONH-heterocycloalkyl, —NHCO— heterocycloalkyl, —NH-heterocycloalkyl, —O-heterocycloalkyl, —CO-heterocycloalkyl, —SO₂-heterocycloalkyl, —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, —SO₂—(C₀₋₂ alkylene)-aryl, —(C₀₋₂ alkylene)-SO₂-aryl, —CONH-aryl, —NHCO-aryl, —NH-aryl, —O-aryl, —CO-aryl, —SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)heteroaryl, —(C₀₋₂ alkylene)-NH-heteroaryl, —O—(C₀₋₂ alkylene)heteroaryl, —(C₀₋₂ alkylene)-O-heteroaryl, —SO₂—(C₀₋₂ alkylene)heteroaryl, —(C₀₋₂ alkylene)-SO₂-heteroaryl, —CONH-heteroaryl, —NHCO-heteroaryl, —NH-heteroaryl, —O-heteroaryl, —CO-heteroaryl, and —SO₂-heteroaryl, wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

33. The compound of any one of claims 1 to 32, wherein R₄ is selected from —(C₀₋₂ alkylene)-aryl, —CO—(C₀₋₂ alkylene)-aryl, —(C₀₋₂ alkylene)-CO-aryl, —CONH—(C₀₋₂ alkylene)-aryl, —(C₀₋₂ alkylene)-CONH-aryl, —NHCO—(C₀₋₂ alkylene)-aryl, —(C₀₋₂ alkylene)-NHCO-aryl, —NH—(C₀₋₂ alkylene)-aryl, —(C₀₋₂ alkylene)-NH-aryl, —O—(C₀₋₂ alkylene)-aryl, —(C₀₋₂ alkylene)-O-aryl, —SO₂—(C₀₋₂ alkylene)-aryl, —(C₀₋₂ alkylene)-SO₂-aryl, —CONH-aryl, —NHCO-aryl, —NH-aryl, —O-aryl, —CO-aryl, —SO₂-aryl, —(C₀₋₂ alkylene)-heteroaryl, —CO—(C₀₋₂ alkylene)-heteroaryl, —(C₀₋₂ alkylene)-CO-heteroaryl, —CONH—(C₀₋₂ alkylene)-heteroaryl, —(C₀₋₂ alkylene)-CONH-heteroaryl, —NHCO—(C₀₋₂ alkylene)-heteroaryl, —(C₀₋₂ alkylene)-NHCO-heteroaryl, —NH—(C₀₋₂ alkylene)-heteroaryl, —(C₀₋₂ alkylene)-NH-heteroaryl, —O—(C₀₋₂ alkylene)-heteroaryl, —(C₀₋₂ alkylene)-O-heteroaryl, —SO₂—(C₀₋₂ alkylene)-heteroaryl, —(C₀₋₂ alkylene)-SO₂-heteroaryl, —CONH-heteroaryl, —NHCO-heteroaryl, —NH-heteroaryl, —O-heteroaryl, —CO-heteroaryl, and —SO₂-heteroaryl, wherein said aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

34. The compound of any one of claims 1 to 32, wherein R₄ is selected from C₁₋₁₂ alkyl, C₂₋₁₂ alkenyl, C₂₋₁₂ alkynyl, cycloalkyl, heterocycloalkyl, aryl, and heteroaryl, preferably wherein R₄ is selected from cycloalkyl, heterocycloalkyl, aryl, and heteroaryl, more preferably wherein R₄ is selected from aryl, and heteroaryl, even more preferably wherein R₄ is heteroaryl, wherein said alkyl, alkenyl, or alkynyl is optionally substituted with one or more groups independently selected from halogen, —CN, —OH, —O(C₁₋₅ alkyl), —O(C₁₋₅ haloalkyl), SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein said cycloalkyl, heterocycloalkyl, aryl or heteroaryl is optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl).

35. The compound of claim 34, wherein R₄ is a five membered heteroaryl, optionally substituted with one or more groups independently selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), preferably wherein the five membered heteroaryl is selected from imidazolyl, isoxazolyl, pyrazolyl, 1,2,3-triazolyl, 1,2,4-triazolyl, thiazolyl, 1,2,4-oxadiazolyl, 1,3,4-oxadiazolyl, 1,2,4-thiadiazolyl, or 1,3,4-thiadiazolyl, preferably wherein the five membered heteroaryl is 1,2,4-thiadiazolyl, optionally substituted with one or more groups independently selected from halogen, —CN, —OH, C₁₋₅ alkyl, C₁₋₅ haloalkyl, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CONH₂, —CONH(C₁₋₅ alkyl), and —CON(C₁₋₅ alkyl)(C₁₋₅ alkyl), preferably optionally substituted with C₁₋₅ alkyl, C₁₋₅ haloalkyl, —O(C₁₋₅ alkyl), —SH, —S(C₁₋₅ alkyl), more preferably optionally substituted with C₁₋₅ alkyl, C₁₋₅ haloalkyl, even more preferably optionally substituted with C₁₋₅ haloalkyl, preferably selected from —CH₂F, —CHF₂ and CF₃, most preferably optionally substituted with —CHF₂.

36. The compound of claim 1, selected from: 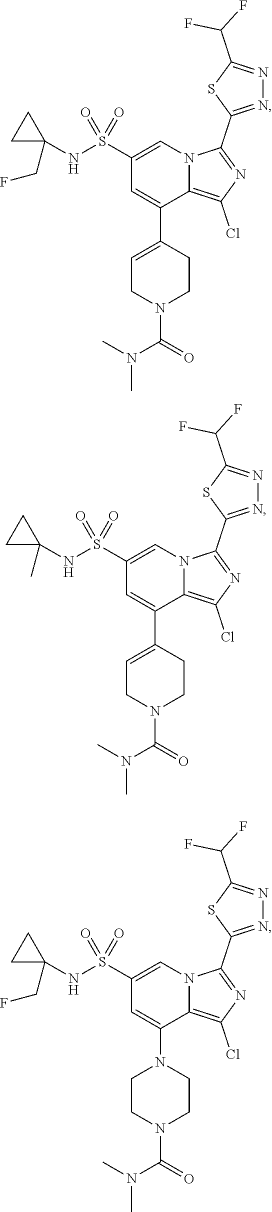 [SMILES: CN(C)C(=O)N1CC=C(c2cc(S(=O)(=O)NC3(CF)CC3)cn3c(-c4nnc(C(F)F)s4)nc(Cl)c23)CC1 ; CN(C)C(=O)N1CC=C(c2cc(S(=O)(=O)NC3(C)CC3)cn3c(-c4nnc(C(F)F)s4)nc(Cl)c23)CC1 ; CN(C)C(=O)N1CCN(c2cc(S(=O)(=O)NC3(CF)CC3)cn3c(-c4nnc(C(F)F)s4)nc(Cl)c23)CC1] 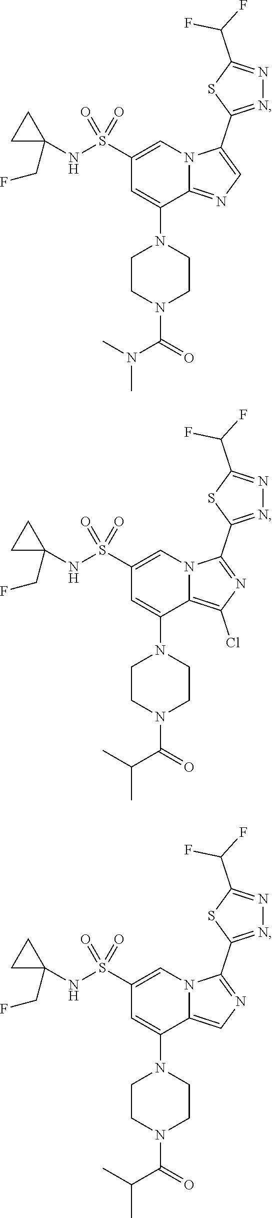 [SMILES: CN(C)C(=O)N1CCN(c2cc(S(=O)(=O)NC3(CF)CC3)cn3c(-c4nnc(C(F)F)s4)cnc23)CC1 ; CC(C)C(=O)N1CCN(c2cc(S(=O)(=O)NC3(CF)CC3)cn3c(-c4nnc(C(F)F)s4)nc(Cl)c23)CC1 ; CC(C)C(=O)N1CCN(c2cc(S(=O)(=O)NC3(CF)CC3)cn3c(-c4nnc(C(F)F)s4)ncc23)CC1] 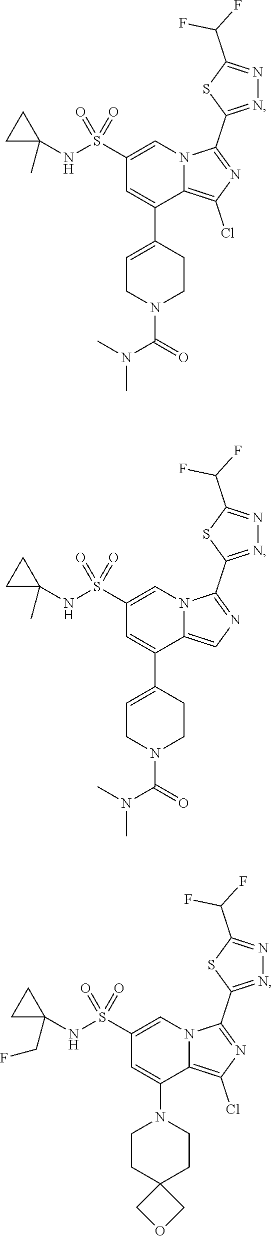 [SMILES: CN(C)C(=O)N1CC=C(c2cc(S(=O)(=O)NC3(C)CC3)cn3c(-c4nnc(C(F)F)s4)nc(Cl)c23)CC1 ; CN(C)C(=O)N1CC=C(c2cc(S(=O)(=O)NC3(C)CC3)cn3c(-c4nnc(C(F)F)s4)ncc23)CC1 ; O=S(=O)(NC1(CF)CC1)c1cc(N2CCC3(CC2)COC3)c2c(Cl)nc(-c3nnc(C(F)F)s3)n2c1] 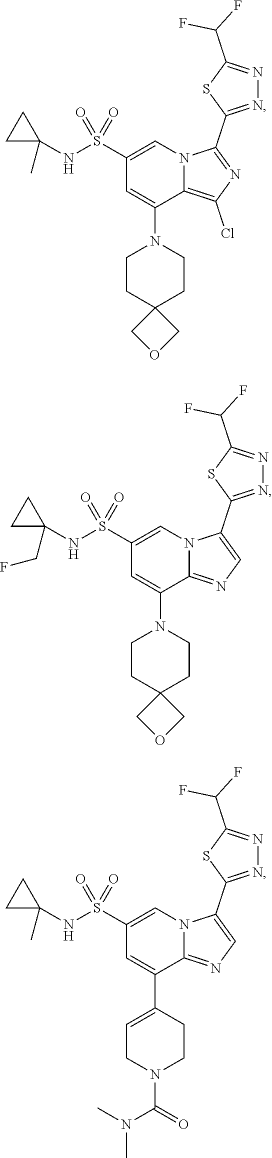 [SMILES: CC1(NS(=O)(=O)c2cc(N3CCC4(CC3)COC4)c3c(Cl)nc(-c4nnc(C(F)F)s4)n3c2)CC1 ; O=S(=O)(NC1(CF)CC1)c1cc(N2CCC3(CC2)COC3)c2ncc(-c3nnc(C(F)F)s3)n2c1 ; CN(C)C(=O)N1CC=C(c2cc(S(=O)(=O)NC3(C)CC3)cn3c(-c4nnc(C(F)F)s4)cnc23)CC1] 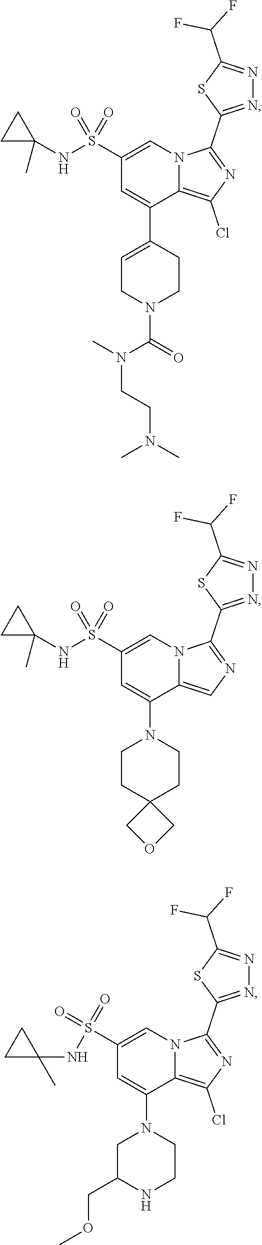 [SMILES: CN(C)CCN(C)C(=O)N1CC=C(c2cc(S(=O)(=O)NC3(C)CC3)cn3c(-c4nnc(C(F)F)s4)nc(Cl)c23)CC1 ; CC1(NS(=O)(=O)c2cc(N3CCC4(CC3)COC4)c3cnc(-c4nnc(C(F)F)s4)n3c2)CC1 ; COCC1CN(c2cc(S(=O)(=O)NC3(C)CC3)cn3c(-c4nnc(C(F)F)s4)nc(Cl)c23)CCN1] 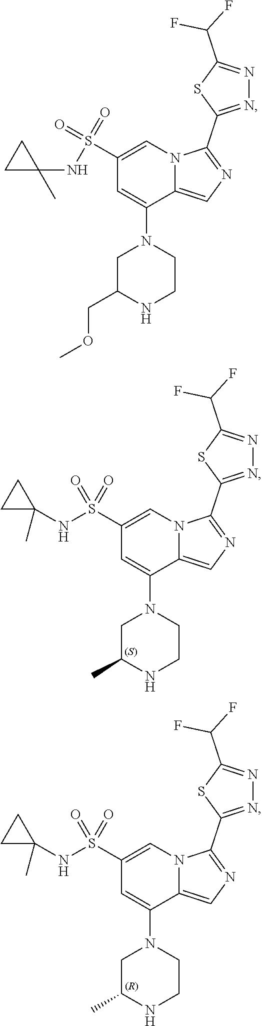 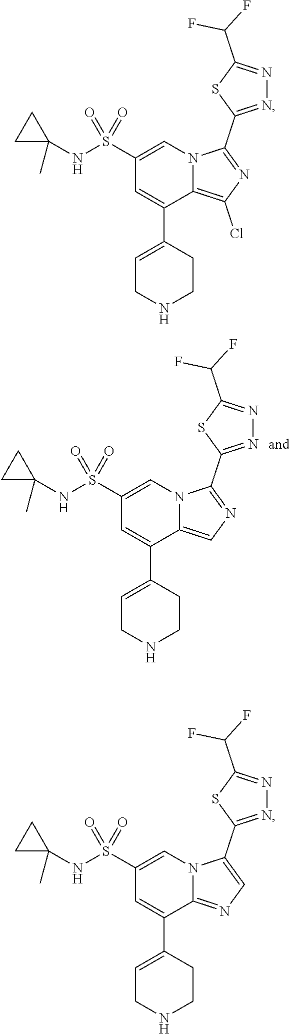 [SMILES: CC1(NS(=O)(=O)c2cc(C3=CCNCC3)c3c(Cl)nc(-c4nnc(C(F)F)s4)n3c2)CC1 ; CC1(NS(=O)(=O)c2cc(C3=CCNCC3)c3cnc(-c4nnc(C(F)F)s4)n3c2)CC1 ; CC1(NS(=O)(=O)c2cc(C3=CCNCC3)c3ncc(-c4nnc(C(F)F)s4)n3c2)CC1]
or a pharmaceutically acceptable salt, hydrate or solvate thereof.

37. A pharmaceutical composition comprising the compound of any one of claims 1 to 36 or a pharmaceutically acceptable salt, hydrate or solvate thereof, and a pharmaceutically acceptable carrier.

38. The compound of any one of claims 1 to 36 or a pharmaceutically acceptable salt, hydrate or solvate thereof, or a pharmaceutical composition of claim 37, for use in therapy.

39. The compound for use or the pharmaceutical composition for use of claim 38, for use in a method of treating a disease or disorder in which PARG activity is implicated.

40. The compound for use or the pharmaceutical composition for use of claim 38, for use in a method of treating a proliferative disorder, preferably wherein the proliferative disorder is cancer, preferably a human cancer.
